(19) United States
(12) Patent Application Publication
(10) Pub. No.: US 2023/0406862 A1
(43) Pub. Date: Dec. 21, 2023

(54) HETEROCYCLIC COMPOUND, ORGANIC ELECTROLUMINESCENT DEVICE INCLUDING SAME, AND COMPOSITION FOR ORGANIC MATERIAL LAYER

(71) Applicant: LT MATERIALS CO., LTD., Yongin-si, Gyeonggi-do (KR)

(72) Inventors: Nam Jin LEE, Yongin-si (KR); Won Jang JEONG, Yongin-si (KR); Yu Jin HEO, Yongin-si (KR); Dong Jun KIM, Yongin-si (KR)

(73) Assignee: LT MATERIALS CO., LTD., Yongin-si, Gyeonggi-do (KR)

(21) Appl. No.: 18/036,069

(22) PCT Filed: Nov. 12, 2021
(86) PCT No.: PCT/KR2021/016552

§ 371 Date: May 9, 2023

(30) Foreign Application Priority Data
Nov. 17, 2020 (KR) 10-2020-0153290

(51) Int. Cl. C07D 493/04; H10K 50/15; H10K 50/16; H10K 50/11; C07D 495/04

(52) U.S. Cl. CPC C07D 493/04; H10K 50/15; H10K 50/16; H10K 50/11; C07D 495/04; C07B 2200/05

(57) ABSTRACT

The present specification relates to a heterocyclic compound represented by Formula 1, an organic light-emitting device comprising the same, and a composition for an organic layer.

### TECHNICAL FIELD

This application claims the benefit of priority based on Korean Patent Application No. 10-2020-0153290 filed on Nov. 17, 2020, and the entire contents disclosed in the literatures of said Korean Patent Application are incorporated as part of the present specification.

The present invention relates to a heterocyclic compound, an organic light-emitting device comprising the same, and a composition for an organic layer.

### BACKGROUND ART

The organic light-emitting device is a type of self-emission type display device, and has advantages that the viewing angle is wide, the contrast is excellent, and the response speed is fast.

The organic light-emitting device has a structure in which an organic thin film is disposed between two electrodes. When a voltage is applied to the organic light-emitting device having such a structure, electrons and holes injected from the two electrodes combine in the organic thin film to form a pair, and then emit light while disappearing. The organic thin film may be composed of a single layer or multiple layers, if necessary.

The organic thin film material may have a light-emitting function, if necessary. For example, as the organic thin film material, a compound capable of constituting the light-emitting layer by itself may be used, or a compound capable of serving as a host or dopant of the host-dopant-based light-emitting layer may be used. In addition, as the organic thin film material, a compound capable of performing the roles of hole injection, hole transport, electron blocking, hole blocking, electron transport, electron injection, and the like may be used.

In order to improve the performance, lifespan, or efficiency of the organic light-emitting device, the development of the organic thin film material is continuously required.

### PRIOR ART REFERENCES

### Patent Documents

Korean Patent Application Laying-Open No. 10-2011-0013445

### DISCLOSURE

### Technical Problem

It is an object of the present invention to provide a heterocyclic compound capable of imparting a low driving voltage, excellent luminous efficiency, and excellent lifespan properties to an organic light-emitting device.

It is another object of the present invention to provide an organic light-emitting device comprising the heterocyclic compound.

It is another object of the present invention to provide a composition for an organic layer comprising the heterocyclic compound.

### Technical Solution

The present invention provides a heterocyclic compound represented by following Formula 1:

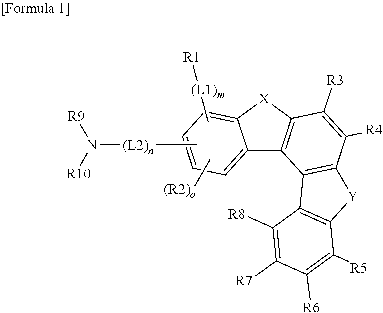

wherein:
X and Y are the same as or different from each other and each independently O or S;
R1 to R8 are the same as or different from each other and are each independently selected from the group consisting of hydrogen, deuterium, halogen, a cyano group, a substituted or unsubstituted C1 to C60 alkyl group, a substituted or unsubstituted C2 to C60 alkenyl group, a substituted or unsubstituted C2 to C60 alkynyl group, a substituted or unsubstituted C1 to C60 alkoxy group, a substituted or unsubstituted C3 to C60 cycloalkyl group, a substituted or unsubstituted C2 to C60 heterocycloalkyl group, a substituted or unsubstituted C6 to C60 aryl group, a substituted or unsubstituted C2 to C60 heteroaryl group, —P(═O)R101R102R103, and —NR101R102, wherein R101, R102, and R103 are the same as or different from each other and are each independently a substituted or unsubstituted C1 to C60 alkyl group, a substituted or unsubstituted C6 to C60 aryl group, or a substituted or unsubstituted C2 to C60 heteroaryl group; or two or more groups adjacent to each other combine with each other to form a substituted or unsubstituted C6 to C60 aromatic hydrocarbon ring or a substituted or unsubstituted C2 to C60 heterocycle;
R9 and R10 are the same as or different from each other and are each independently a substituted or unsubstituted C6 to C60 aryl group, or a substituted or unsubstituted C2 to C60 heteroaryl group;
L1 and L2 are the same as or different from each other and are each independently a direct bond, a substituted or unsubstituted C6 to C60 arylene group, or a substituted or unsubstituted C2 to C60 heteroarylene group;
m is an integer of 0 to 4, provided that when m is 0, L1 is a direct bond, and when m is 2 to 4, each L1 is the same as or different from each other and is each independently selected;
n is an integer of 0 to 4, provided that when n is 0, L2 is a direct bond, and when n is 2 to 4, each L2 is the same as or different from each other and is each independently selected;
is an integer of 0 to 2, provided that when o is 2, each R2 is the same as or different from each other and is each independently selected.

In addition, the present invention provides an organic light-emitting device comprising:
a first electrode;
a second electrode provided to face the first electrode; and
one or more organic layers provided between the first electrode and the second electrode, and
wherein one or more of the organic layers comprise the heterocyclic compound represented by Formula 1.

In addition, the present invention provides a composition for an organic layer of an organic light-emitting device, comprising the heterocyclic compound represented by Formula 1.

### Advantageous Effects

The heterocyclic compound of the present invention and the composition for an organic layer comprising the same may be usefully used as a material for an organic layer of an organic light-emitting device. In particular, these are used as a hole transport layer material, an electron-blocking layer material, and a light-emitting layer material, thereby providing remarkable effects of lowering the driving voltage of the organic light-emitting device, improving the luminous efficiency, and improving the lifespan properties.

The organic light-emitting device of the present invention comprises the heterocyclic compound or the composition for an organic layer comprising the same, thereby providing excellent driving voltage, luminous efficiency, and lifespan properties.

### DESCRIPTION OF DRAWINGS

FIGS. 1 to 3 are schematic views showing a stacked structure of an organic light-emitting device according to one embodiment of the present invention, respectively.

### BEST MODE

Hereinafter, the present invention will be described in detail.

In the present invention, the term “substituted” means that a hydrogen atom bonded to a carbon atom of a compound is replaced with another substituent, and the position to be substituted is not limited as long as it is the position at which a hydrogen atom is substituted, that is, the position at which the substituent is substitutable. When two or more substituents are substituted, the two or more substituents may be the same as or different from each other.

In the present invention, the term “substituted or unsubstituted” means that it is unsubstituted or substituted by one or more substituents selected from the group consisting of C1 to C60 linear or branched alkyl, C2 to C60 linear or branched alkenyl, C2 to C60 linear or branched alkynyl, C3 to C60 monocyclic or polycyclic cycloalkyl, C2 to C60 monocyclic or polycyclic heterocycloalkyl, C6 to C60 monocyclic or polycyclic aryl, C2 to C60 monocyclic or polycyclic heteroaryl, —SiRR′R″, —P(═O)RR′, C1 to C20 alkylamine, C6 to C60 monocyclic or polycyclic arylamine, and C2 to C60 monocyclic or polycyclic heteroarylamine; or it is unsubstituted or substituted by a substituent to which two or more substituents selected from the above-exemplified substituents are connected.

In the present invention, the alkyl group includes a linear or branched chain having 1 to 60 carbon atoms, and may be further substituted by another substituent. The number of carbon atoms in the alkyl group may be 1 to 60, specifically 1 to 40, more specifically 1 to 20. Specific examples include, but are not limited to, methyl group, ethyl group, propyl group, n-propyl group, isopropyl group, butyl group, n-butyl group, isobutyl group, tert-butyl group, sec-butyl group, 1-methyl-butyl group, 1-ethyl-butyl group, pentyl group, n-pentyl group, isopentyl group, neopentyl group, tert-pentyl group, hexyl group, n-hexyl group, 1-methylpentyl group, 2-methylpentyl group, 4-methyl-2-pentyl group, 3,3-dimethylbutyl group, 2-ethylbutyl group, heptyl group, n-heptyl group, 1-methylhexyl group, cyclopentylmethyl group, cyclohexylmethyl group, octyl group, n-octyl group, tert-octyl group, 1-methylheptyl group, 2-ethylhexyl group, 2-propylpentyl group, n-nonyl group, 2,2-dimethylheptyl group, 1-ethyl-propyl group, 1,1-dimethyl-propyl group, isohexyl group, 2-methylpentyl group, 4-methylhexyl group, 5-methylhexyl group, and the like.

In the present invention, the alkenyl group includes a linear or branched chain having 2 to 60 carbon atoms, and may be further substituted by another substituent. The number of carbon atoms in the alkenyl group may be 2 to 60, specifically 2 to 40, more specifically 2 to 20. Specific examples include, but are not limited to, a vinyl group, 1-propenyl group, isopropenyl group, 1-butenyl group, 2-butenyl group, 3-butenyl group, 1-pentenyl group, 2-pentenyl group, 3-pentenyl group, 3-methyl-1-butenyl group, 1,3-butadienyl group, allyl group, 1-phenylvinyl-1-yl group, 2-phenylvinyl-1-yl group, 2,2-diphenylvinyl-1-yl group, 2-phenyl-2-(naphthyl-1-yl)vinyl-1-yl group, 2,2-bis(diphenyl-1-yl)vinyl-1-yl group, stilbenyl group, styrenyl group, and the like.

In the present invention, the alkynyl group includes a linear or branched chain having 2 to 60 carbon atoms, and may be further substituted by another substituent. The number of carbon atoms in the alkynyl group may be 2 to 60, specifically 2 to 40, more specifically 2 to 20.

In the present invention, the cycloalkyl group includes a monocyclic or polycyclic ring having 3 to 60 carbon atoms, and may be further substituted by another substituent. Herein, the polycyclic refers to a group in which a cycloalkyl group is directly connected or condensed with another cyclic group. Herein, the another cyclic group may be a cycloalkyl group, but may be a different type of cyclic group, for example, a heterocycloalkyl group, an aryl group, a heteroaryl group, and the like. The number of carbon atoms in the cycloalkyl group may be 3 to 60, specifically 3 to 40, more specifically 5 to 20. Specific examples include, but are not limited to, cyclopropyl group, cyclobutyl group, cyclopentyl group, 3-methylcyclopentyl group, 2,3-dimethylcyclopentyl group, cyclohexyl group, 3-methylcyclohexyl group, 4-methylcyclohexyl group, 2,3-dimethylcyclohexyl group, 3,4,5-trimethylcyclohexyl group, 4-tert-butylcyclohexyl group, cycloheptyl group, cyclooctyl group, and the like.

In the present invention, the heterocycloalkyl group includes O, S, Se, N, or Si as a heteroatom, includes a monocyclic or polycyclic ring having 2 to 60 carbon atoms, and may be further substituted by another substituent. Herein, polycyclic refers to a group in which a heterocycloalkyl group is directly connected or condensed with another cyclic group. Herein, another cyclic group may be a heterocycloalkyl group, but may be a different type of cyclic group, for example, a cycloalkyl group, an aryl group, a heteroaryl group, and the like. The number of carbon atoms in the heterocycloalkyl group may be 2 to 60, specifically 2 to 40, more specifically 3 to 20.

In the present invention, the aryl group includes a monocyclic or polycyclic ring having 6 to 60 carbon atoms, and may be further substituted by other substituents. Herein, the polycyclic refers to a group in which an aryl group is directly connected or condensed with another cyclic group. Herein, the another cyclic group may be an aryl group, but may be a different type of cyclic group, for example, a cycloalkyl group, a heterocycloalkyl group, a heteroaryl group, and the like. The aryl group includes a spiro group. The number of carbon atoms in the aryl group may be 6 to 60, specifically 6 to 40, more specifically 6 to 25. Specific examples of the aryl group may include, but are not limited to, a phenyl group, a biphenyl group, a triphenyl group, a naphthyl group, an anthryl group, a chrysenyl group, a phenanthrenyl group, a perylenyl group, a fluoranthenyl group, a triphenylenyl group, a phenalenyl group, a pyrenyl group, tetracenyl group, pentacenyl group, fluorenyl group, indenyl group, acenaphthylenyl group, benzofluorenyl group, spirobifluorenyl group, 2,3-dihydro-1H-indenyl group, condensed cyclic groups thereof, and the like.

In the present invention, the fluorenyl group may be substituted, and adjacent substituents may be bonded to each other to form a ring.

When the fluorenyl group is substituted, it may be, but is not limited to,

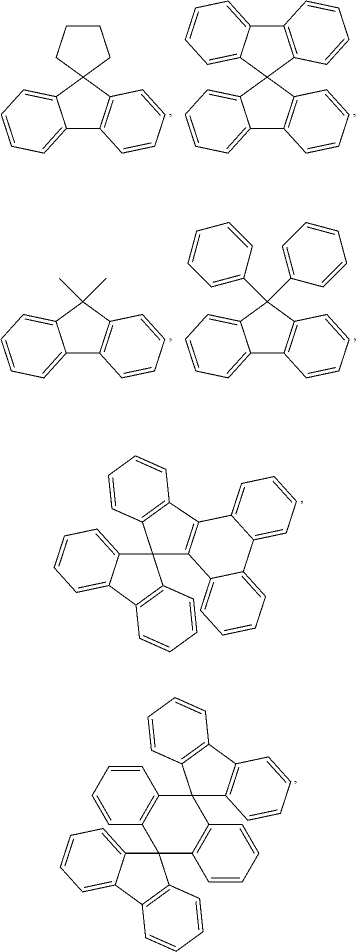

and the like.

In the present invention, the heteroaryl group includes S, O, Se, N, or Si as a heteroatom, includes a monocyclic or polycyclic ring having 2 to 60 carbon atoms, and may be further substituted by other substituents. Herein, the polycyclic refers to a group in which a heteroaryl group is directly connected or condensed with another cyclic group. Herein, the another cyclic group may be a heteroaryl group, but may be a different type of cyclic group, for example, a cycloalkyl group, a heterocycloalkyl group, an aryl group, and the like. The number of carbon atoms in the heteroaryl group may be 2 to 60, specifically 2 to 40, more specifically 3 to 25. Specific examples of the heteroaryl group may include, but are not limited to, a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolylyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophenyl group, a benzofuranyl group, a dibenzothiophenyl group, a dibenzofuranyl group, a carbazolyl group, a benzocarbazolyl group, a dibenzocarbazolyl group, a phenazinyl group, a dibenzosilol group, a spirobi(dibenzosilole) group, a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepinyl group, a 9,10-dihydroacridinyl group, a phenanthrazinyl group, a phenothiazinyl group, a phthalazinyl group, a naphthylidinyl group, a phenanthrolinyl group, a benzo[c] [1,2,5]thiadiazolyl group, a 5,10-dihydrodibenzo[b, e] [1,4] azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like.

In the present invention, the amine group may be selected from the group consisting of a monoalkylamine group, monoarylamine group, monoheteroarylamine group, —NH₂, a dialkylamine group, a diarylamine group, a diheteroarylamine group, an alkylarylamine group, an alkylheteroarylamine group, and an arylheteroarylamine group; and the number of carbon atoms is not particularly limited, but is preferably 1 to 30. Specific examples of the amine group include, but are not limited to, a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like.

In the present invention, the arylene group refers to a group having two bonding positions on the aryl group, that is, a divalent group. The description of the aryl group described above may be applied, except that each of these is a divalent group. In addition, the heteroarylene group refers to a group having two bonding positions on the heteroaryl group, that is, a divalent group. The description of the heteroaryl group described above may be applied, except that each of these is a divalent group.

In the present invention, an “adjacent” group may refer to a substituent substituted on an atom directly connected to the atom on which a certain substituent is substituted, a substituent sterically closest to a certain substituent, or another substituent substituted on the atom in which a certain substituent is substituted. For example, two substituents substituted at an ortho position on a benzene ring and two substituents substituted at the same carbon on an aliphatic ring may be interpreted as “adjacent” groups to each other.

In the present invention, “the case where a substituent is not indicated in the chemical formula or compound structure” means that a hydrogen atom is bonded to a carbon atom. However, since deuterium (²H) is an isotope of hydrogen, some hydrogen atoms may be deuterium.

In one embodiment of the present invention, “the case where a substituent is not indicated in the chemical formula or compound structure” may mean that hydrogen or deuterium is present at all positions that may be substituted with a substituent. That is, since deuterium is an isotope of hydrogen, some hydrogen atoms may be isotope deuterium and the content of deuterium may be 0% to 100%.

In one embodiment of the present invention, in “the case where a substituent is not indicated in the chemical formula or compound structure,” hydrogen and deuterium may be used interchangeably in compounds unless deuterium is explicitly excluded, such as “the content of deuterium is 0%,” “the content of hydrogen is 100%,” and “all substituents are hydrogen.” In one embodiment of the present invention, deuterium is one of the isotopes of hydrogen and is an element having a deuteron consisting of one proton and one neutron as a nucleus, and may be expressed as hydrogen-2, and its element symbol may also be written as D or ²H.

In one embodiment of the present invention, isotopes, which refer to atoms having the same atomic number (Z) but different mass numbers (A), may also be interpreted as elements having the same number of protons but a different number of neutrons.

In one embodiment of the present invention, the meaning of the T % content of a specific substituent may be defined as the following formula: T2/T1×100=T %, wherein T1 is defined as the total number of substituents that the basic compound can have and T2 is defined as the number of a specific substituent.

That is, in one example, the 20% content of deuterium in the phenyl group represented by

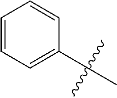 [SMILES: *c1ccccc1]

may mean that the total number of substituents that the phenyl group can have is 5 (T1 in the formula) and the number of deuterium is 1 (T2 in the formula). That is, the 20% content of deuterium in the phenyl group may be represented by the following structural formula:

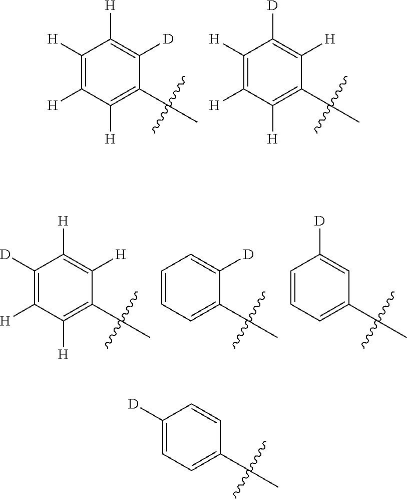

In addition, in one embodiment of the present invention, the case of “a phenyl group having a deuterium content of 0%” may mean a phenyl group that does not contain deuterium atoms, that is, has 5 hydrogen atoms.

In the present invention, the content of deuterium in the heterocyclic compound represented by Formula 1 may be 0 to 100%, more preferably 60 to 100%.

In the present invention, C6 to C60 aromatic hydrocarbon ring refers to a compound including an aromatic ring consisting of C6 to C60 carbons and hydrogens, and includes, but is not limited to, for example, benzene, biphenyl, triphenyl, triphenylene, naphthalene, anthracene, phenalene, phenanthrene, fluorene, pyrene, chrysene, perylene, azulene, and the like, and includes all aromatic hydrocarbon ring compounds known in the art that satisfy the above carbon number.

The present invention provides a heterocyclic compound represented by following Formula 1:

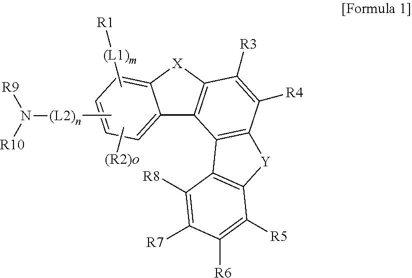

wherein:
X and Y are the same as or different from each other and each independently O or S;
R1 to R8 are the same as or different from each other and are each independently selected from the group consisting of hydrogen, deuterium, halogen, a cyano group, a substituted or unsubstituted C1 to C60 alkyl group, a substituted or unsubstituted C2 to C60 alkenyl group, a substituted or unsubstituted C2 to C60 alkynyl group, a substituted or unsubstituted C1 to C60 alkoxy group, a substituted or unsubstituted C3 to C60 cycloalkyl group, a substituted or unsubstituted C2 to C60 heterocycloalkyl group, a substituted or unsubstituted C6 to C60 aryl group, a substituted or unsubstituted C2 to C60 heteroaryl group, —P(═O)R101R102R103, and —NR101R102, wherein R101, R102, and R103 are the same as or different from each other and are each independently a substituted or unsubstituted C1 to C60 alkyl group, a substituted or unsubstituted C6 to C60 aryl group, or a substituted or unsubstituted C2 to C60 heteroaryl group; or two or more groups adjacent to each other combine with each other to form a substituted or unsubstituted C6 to C60 aromatic hydrocarbon ring or a substituted or unsubstituted C2 to C60 heterocycle;
R9 and R10 are the same as or different from each other and are each independently a substituted or unsubstituted C6 to C60 aryl group, or a substituted or unsubstituted C2 to C60 heteroaryl group;
L1 and L2 are the same as or different from each other and are each independently a direct bond, a substituted or unsubstituted C6 to C60 arylene group, or a substituted or unsubstituted C2 to C60 heteroarylene group;
m is an integer of 0 to 4, provided that when m is 0, L1 is a direct bond, and when m is 2 to 4, each L1 is the same as or different from each other and is each independently selected;
n is an integer of 0 to 4, provided that when n is 0, L2 is a direct bond, and when n is 2 to 4, each L2 is the same as or different from each other and is each independently selected;
is an integer of 0 to 2, provided that when o is 2, each R2 is the same as or different from each other and is each independently selected.

In Formula 1 above, X may be 0, and X may be S.

In Formula 1 above, Y may be 0, and Y may be S.

In one embodiment of the present invention, the heteroatom in the heteroatom-containing group may be one or more selected from O, S, Se, N, or Si.

In another embodiment of the present invention, the heteroatom in the heteroatom-containing group may be one or more selected from O, S, or N.

In one embodiment of the present invention, R1 may be hydrogen, deuterium, a substituted or unsubstituted C1 to C60 alkyl group, a substituted or unsubstituted C6 to C60 aryl group, a substituted or unsubstituted C2 to C60 heteroaryl group, or —P(═O)R101R102R103, wherein R101, R102, and R103 may be the same as or different from each other and may be each independently a substituted or unsubstituted C6 to C60 aryl group or a substituted or unsubstituted C2 to C60 heteroaryl group.

In another embodiment of the present invention, R1 may be a substituted or unsubstituted C6 to C30 aryl group, a substituted or unsubstituted C2 to C30 heteroaryl group, or —P(═O)R101R102R103, wherein R101, R102, and R103 may be the same as or different from each other and may be each independently a substituted or unsubstituted C6 to C30 aryl group or a substituted or unsubstituted C2 to C30 heteroaryl group.

In another embodiment of the present invention, R1 may be phenyl, biphenyl, naphthalenyl, fluorenyl, 9,9-dimethylfluorenyl group, 9,9-diphenylfluorenyl group, spirobifluorenyl, phenanthrenyl, triphenylenyl, dibenzothiophenyl, or dibenzofuranyl group, wherein the substituents may be in a “substituted or unsubstituted” form.

In another embodiment of the present invention, R1 may be phenyl, biphenyl, naphthyl, phenanthrenyl, triphenylenyl, 9,9-dimethylfluorenyl, dibenzothiophenyl, or dibenzofuranyl groups, wherein the substituents may be in a “substituted or unsubstituted” form.

In one embodiment of the present invention, R2 to R8 may be the same as or different from each other and may be each independently hydrogen, deuterium, halogen, a cyano group, a substituted or unsubstituted C1 to C20 alkyl group, a substituted or unsubstituted C2 to C20 alkenyl group, a substituted or unsubstituted C6 to C30 aryl group, a substituted or unsubstituted C2 to C30 heteroaryl group, or —P(═O)R101R102R103, wherein R101, R102, and R103 may be the same as or different from each other and may be each independently a substituted or unsubstituted C6 to C30 aryl group or a substituted or unsubstituted C2 to C30 heteroaryl group.

In another embodiment of the present invention, R2 to R8 may be the same as or different from each other and may be each independently hydrogen, deuterium, a substituted or unsubstituted C1 to C10 alkyl group, a substituted or unsubstituted C6 to C20 aryl group, a substituted or unsubstituted C2 to C20 heteroaryl group, or —P(═O)R101R102R103, wherein R101, R102, and R103 may be the same as or different from each other and may be each independently a substituted or unsubstituted C6 to C20 aryl group or a substituted or unsubstituted C2 to C20 heteroaryl group.

In another embodiment of the present invention, R2 to R8 may be the same as or different from each other and may be each independently hydrogen, deuterium, a substituted or unsubstituted C1 to C5 alkyl group, a substituted or unsubstituted C6 to C20 aryl group, or a substituted or unsubstituted C2 to C20 heteroaryl group.

In another embodiment of the present invention, R2 to R8 may be the same as or different from each other and may be each independently hydrogen, deuterium, a substituted or unsubstituted C1 to C5 alkyl group, a substituted or unsubstituted phenyl group, a substituted or unsubstituted biphenyl group, a substituted or unsubstituted naphthalenyl group, a substituted or unsubstituted fluorenyl group, a substituted or unsubstituted 9,9-dimethylfluorenyl group, a substituted or unsubstituted phenanthrenyl group, a substituted or unsubstituted triphenylenyl group, a substituted or unsubstituted dibenzothiophenyl group, or a substituted or unsubstituted dibenzofuranyl group.

In another embodiment of the present invention, R2 to R8 may be the same as or different from each other and may be hydrogen or deuterium.

In one embodiment of the present invention, R9 and R10 may be the same as or different from each other and may be each independently a substituted or unsubstituted C6 to C30 aryl group, or a substituted or unsubstituted C2 to C30 heteroaryl group.

In one embodiment of the present invention, R9 and R10 may be the same as or different from each other and may be each independently phenyl, biphenyl, naphthalenyl, fluorenyl, 9,9-dimethylfluorenyl group, 9,9-diphenylfluorenyl group, spirobifluorenyl, phenanthrenyl, triphenylenyl, dibenzothiophenyl, or dibenzofuranyl group, wherein the substituents may be in a “substituted or unsubstituted” form.

In one embodiment of the present invention, R9 and R10 may be the same as or different from each other and may be each independently phenyl, biphenyl, naphthyl, 9,9-dimethylfluorenyl, 9,9-diphenylfluorenyl, or spirobifluorenyl groups, wherein the substituents may be in a “substituted or unsubstituted” form.

In one embodiment of the present invention, L1 to L8 may be the same as or different from each other and may be each independently a direct bond, a substituted or unsubstituted C6 to C30 arylene group, or a substituted or unsubstituted C2 to C30 heteroarylene group.

In another embodiment of the present invention, L1 to L8 may be the same as or different from each other and may be each independently a direct bond, a substituted or unsubstituted C6 to C20 arylene group, or a substituted or unsubstituted C2 to C20 heteroarylene group.

In another embodiment of the present invention, L1 to L8 may be the same as or different from each other and may be each independently a direct bond, phenyl, biphenyl, naphthalenyl, phenanthrenyl, or triphenylenyl groups, wherein the substituents may be in a “substituted or unsubstituted” form.

In another embodiment of the present invention, L1 to L8 may be the same as or different from each other and may be each independently a direct bond, phenyl, or biphenyl groups, wherein the substituents may be in a “substituted or unsubstituted” form.

In one embodiment of the present invention, substituents substituted on R1 to R10, L1, and L2 may be the same as or different from each other and may be each independently composed of one or more substituents each independently selected from the group consisting of C1 to C10 linear or branched alkyl, C2 to C10 linear or branched alkenyl, C2 to C10 linear or branched alkynyl, C3 to C15 cycloalkyl, C2 to C20 heterocycloalkyl, C6 to C30 aryl, C2 to C30 heteroaryl, C1 to C10 alkylamine, C6 to C30 arylamine, and C2 to C30 heteroarylamine.

In another embodiment of the present invention, substituents substituted on R1 to R10, L1, and L2 may be the same as or different from each other and may be each independently composed of one or more substituents each independently selected from the group consisting of C1 to C10 linear or branched alkyl, C2 to C10 linear or branched alkenyl, C2 to C10 linear or branched alkynyl, C6 to C30 aryl, C2 to C30 heteroaryl, C6 to C30 arylamine, and C2 to C30 heteroarylamine.

In another embodiment of the present invention, substituents substituted on R1 to R10, L1, and L2 may be the same as or different from each other and may be each independently composed of one or more substituents each independently selected from the group consisting of C1 to C10 linear or branched alkyl, C6 to C30 aryl, C2 to C30 heteroaryl, C6 to C30 arylamine, and C2 to C30 heteroarylamine.

In another embodiment of the present invention, substituents substituted on R1 to R10, L1, and L2 may be the same as or different from each other and may be each independently composed of one or more substituents each independently selected from the group consisting of a C1 to C5 linear or branched alkyl, phenyl, naphthalenyl, pyridinyl, anthracenyl, carbazole, biphenyl, dibenzothiophene, dibenzofuran, and phenanthrenyl.

In one embodiment of the present invention, in Formula 1, m is an integer of 0 to 3, provided that when m is 0, L1 is a direct bond, and when m is 2 to 3, each L1 is the same as or different from each other and is each independently selected; and n is an integer of 0 to 3, provided that when n is 0, L2 is a direct bond, and when n is 2 to 3, each L2 is the same as or different from each other and may be each independently selected.

In another embodiment of the present invention, in Formula 1, m is an integer of 0 to 2, provided that when m is 0, L1 is a direct bond, and when m is 2, each L1 is the same as or different from each other and is each independently selected; and n is an integer of 0 to 2, provided that when n is 0, L2 is a direct bond, and when n is 2, each L2 is the same as or different from each other and may be each independently selected.

In another embodiment of the present invention, in Formula 1, m is an integer of 0 or 1, provided that when m is 0, L1 is a direct bond; and n is an integer of 0 to 1, provided that when n is 0, L2 may be a direct bond.

In one embodiment of the present invention, in Formula 1, o may be 0, 1, or 2.

It may be preferable to use the compound of Formula 1, wherein
X and Y are 0 or S;
R1, R9, and R10 may be the same as or different from each other and may be each independently phenyl, biphenyl, naphthalenyl, fluorenyl, 9,9-dimethylfluorenyl group, 9,9-diphenylfluorenyl group, spirobifluorenyl, phenanthrenyl, triphenylenyl, dibenzothiophenyl, or dibenzofuranyl group, wherein the substituents may be in a “substituted or unsubstituted” form;
in addition, R2 to R8 may be the same as or different from each other and may be hydrogen or deuterium;
in addition, L1 and L2 are a direct bond or phenylene;
m and n are the same as or different from each other and are each independently an integer of 0 to 2; and
is 1.

It may be more preferable to use the compound of Formula 1, wherein
X and Y are 0 or S;
R1 is substituted or unsubstituted phenyl, biphenyl, naphthyl, phenanthrenyl, triphenylenyl, 9,9-dimethylfluorenyl, dibenzothiophenyl, or dibenzofuranyl;
R2 to R8 are the same as or different from each other and are each independently hydrogen or deuterium;
R9 and R10 are the same as or different from each other and are each independently substituted or unsubstituted phenyl, biphenyl, naphthyl, 9,9-dimethylfluorenyl, 9,9-diphenylfluorenyl, or spirobifluorenyl;
L1 and L2 are a direct bond or phenylene;
m and n are the same as or different from each other and are each independently an integer of 0 to 3; and
is 0.

Even more preferably, wherein X and Y may be 0.

In one embodiment of the present invention, the heterocyclic compound represented by Formula 1 may be a compound represented by any one of the following compounds:

Compound:

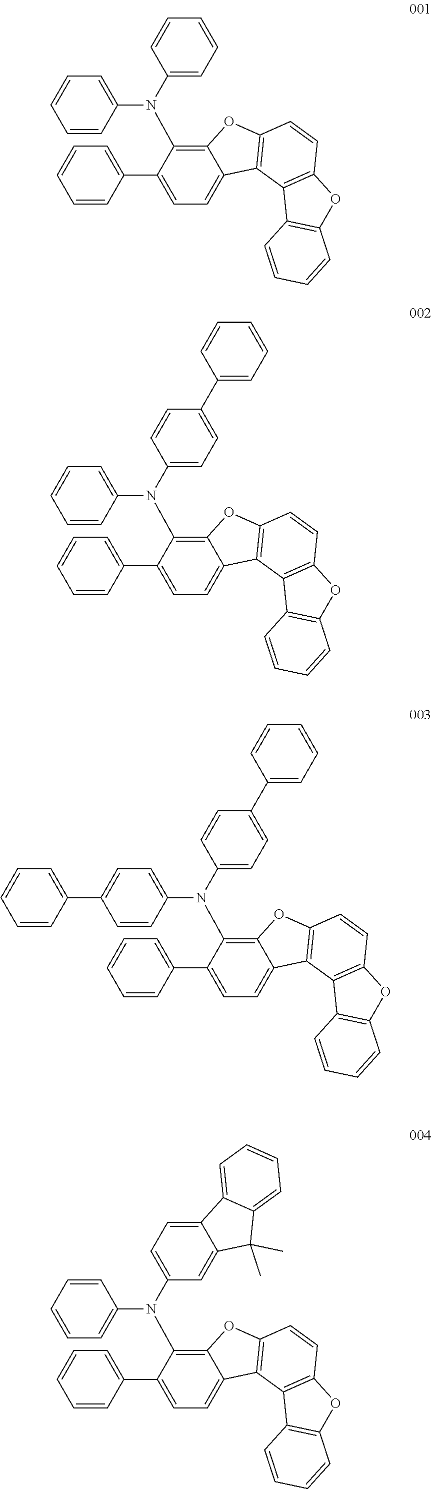 [SMILES: c1ccc(-c2ccc3c(oc4ccc5oc6ccccc6c5c43)c2N(c2ccccc2)c2ccccc2)cc1 ; c1ccc(-c2ccc(N(c3ccccc3)c3c(-c4ccccc4)ccc4c3oc3ccc5oc6ccccc6c5c34)cc2)cc1 ; c1ccc(-c2ccc(N(c3ccc(-c4ccccc4)cc3)c3c(-c4ccccc4)ccc4c3oc3ccc5oc6ccccc6c5c34)cc2)cc1 ; CC1(C)c2ccccc2-c2ccc(N(c3ccccc3)c3c(-c4ccccc4)ccc4c3oc3ccc5oc6ccccc6c5c34)cc21]

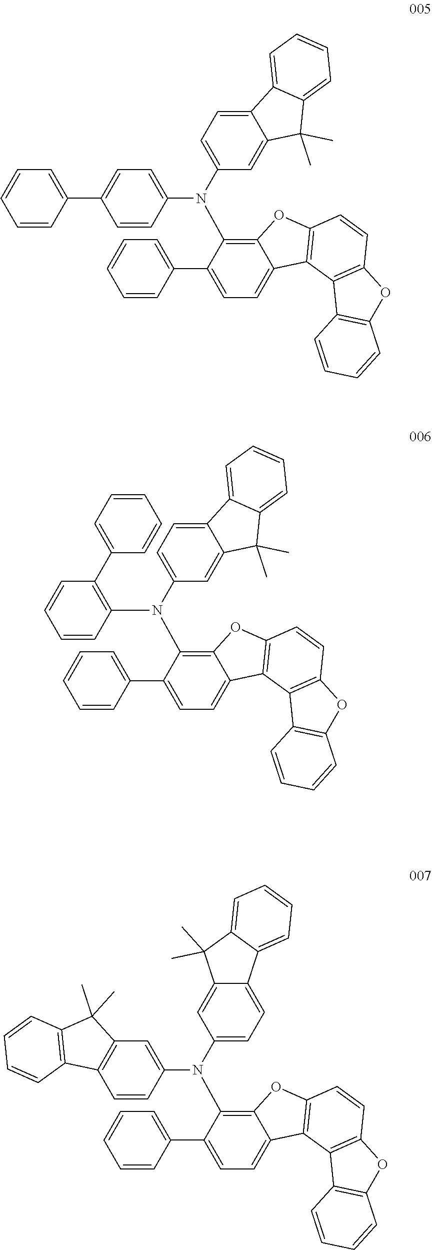 [SMILES: CC1(C)c2ccccc2-c2ccc(N(c3ccc(-c4ccccc4)cc3)c3c(-c4ccccc4)ccc4c3oc3ccc5oc6ccccc6c5c34)cc21 ; CC1(C)c2ccccc2-c2ccc(N(c3ccccc3-c3ccccc3)c3c(-c4ccccc4)ccc4c3oc3ccc5oc6ccccc6c5c34)cc21 ; CC1(C)c2ccccc2-c2ccc(N(c3ccc4c(c3)C(C)(C)c3ccccc3-4)c3c(-c4ccccc4)ccc4c3oc3ccc5oc6ccccc6c5c34)cc21]

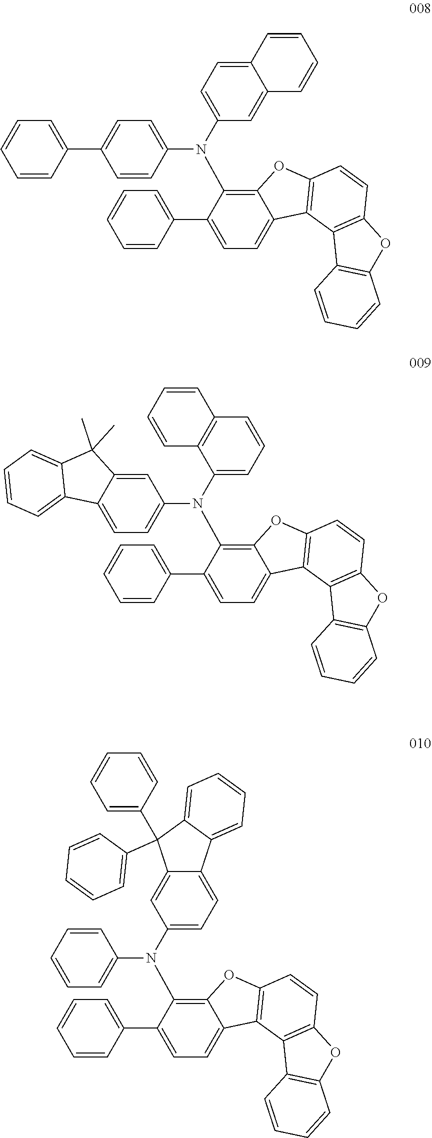 [SMILES: c1ccc(-c2ccc(N(c3ccc4ccccc4c3)c3c(-c4ccccc4)ccc4c3oc3ccc5oc6ccccc6c5c34)cc2)cc1 ; CC1(C)c2ccccc2-c2ccc(N(c3cccc4ccccc34)c3c(-c4ccccc4)ccc4c3oc3ccc5oc6ccccc6c5c34)cc21 ; c1ccc(-c2ccc3c(oc4ccc5oc6ccccc6c5c43)c2N(c2ccccc2)c2ccc3c(c2)C(c2ccccc2)(c2ccccc2)c2ccccc2-3)cc1]

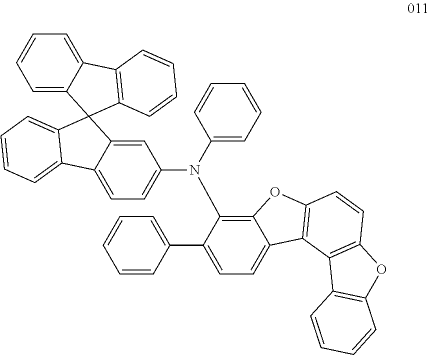 [SMILES: c1ccc(-c2ccc3c(oc4ccc5oc6ccccc6c5c43)c2N(c2ccccc2)c2ccc3c(c2)C2(c4ccccc4-c4ccccc42)c2ccccc2-3)cc1]

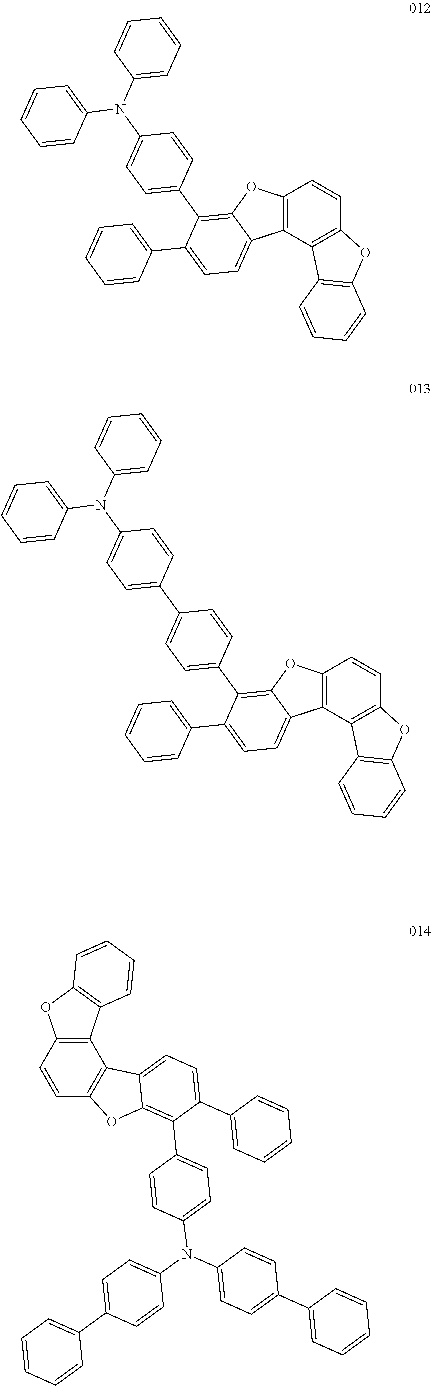 [SMILES: c1ccc(-c2ccc3c(oc4ccc5oc6ccccc6c5c43)c2-c2ccc(N(c3ccccc3)c3ccccc3)cc2)cc1 ; c1ccc(-c2ccc3c(oc4ccc5oc6ccccc6c5c43)c2-c2ccc(-c3ccc(N(c4ccccc4)c4ccccc4)cc3)cc2)cc1 ; c1ccc(-c2ccc(N(c3ccc(-c4ccccc4)cc3)c3ccc(-c4c(-c5ccccc5)ccc5c4oc4ccc6oc7ccccc7c6c45)cc3)cc2)cc1]

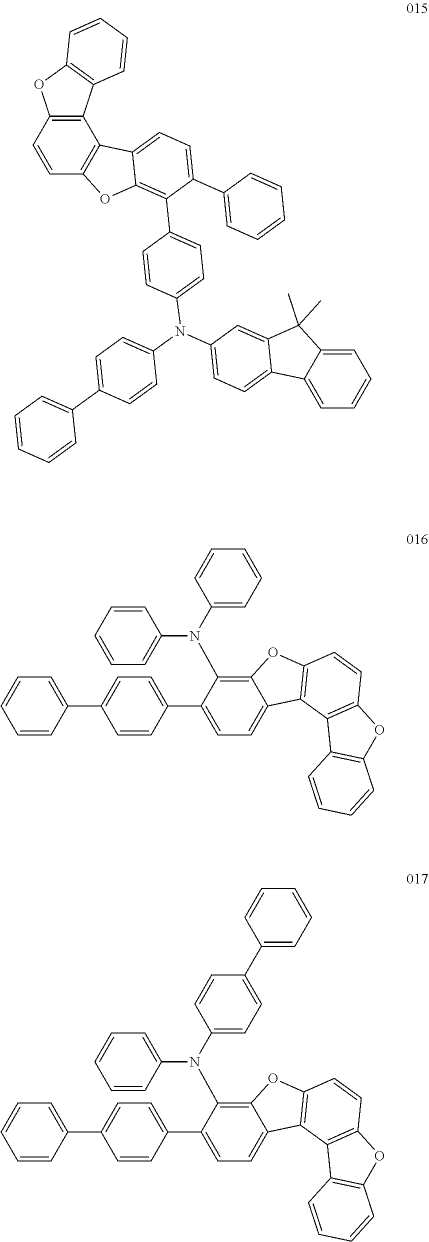 [SMILES: CC1(C)c2ccccc2-c2ccc(N(c3ccc(-c4ccccc4)cc3)c3ccc(-c4c(-c5ccccc5)ccc5c4oc4ccc6oc7ccccc7c6c45)cc3)cc21 ; c1ccc(-c2ccc(-c3ccc4c(oc5ccc6oc7ccccc7c6c54)c3N(c3ccccc3)c3ccccc3)cc2)cc1 ; c1ccc(-c2ccc(-c3ccc4c(oc5ccc6oc7ccccc7c6c54)c3N(c3ccccc3)c3ccc(-c4ccccc4)cc3)cc2)cc1]

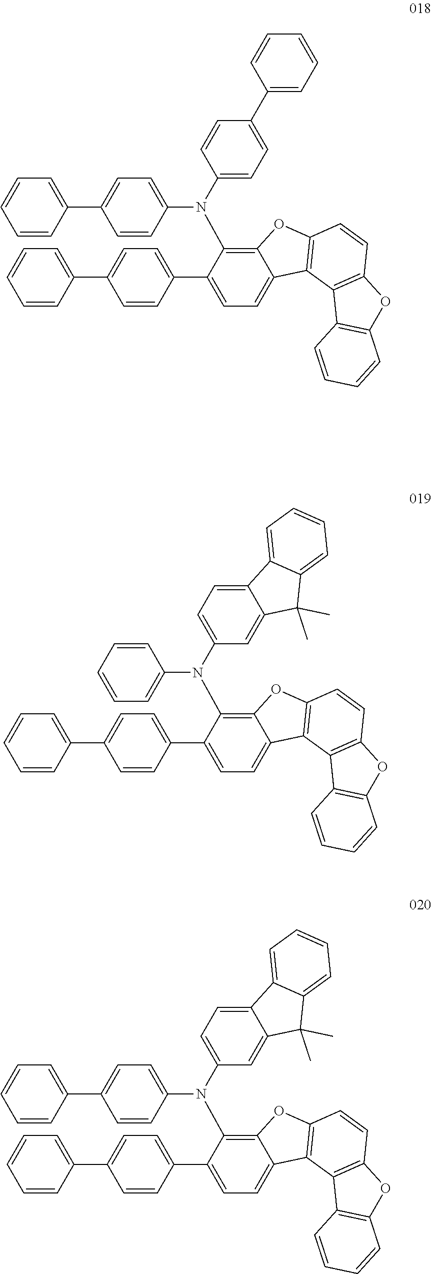 [SMILES: c1ccc(-c2ccc(-c3ccc4c(oc5ccc6oc7ccccc7c6c54)c3N(c3ccc(-c4ccccc4)cc3)c3ccc(-c4ccccc4)cc3)cc2)cc1 ; CC1(C)c2ccccc2-c2ccc(N(c3ccccc3)c3c(-c4ccc(-c5ccccc5)cc4)ccc4c3oc3ccc5oc6ccccc6c5c34)cc21 ; CC1(C)c2ccccc2-c2ccc(N(c3ccc(-c4ccccc4)cc3)c3c(-c4ccc(-c5ccccc5)cc4)ccc4c3oc3ccc5oc6ccccc6c5c34)cc21]

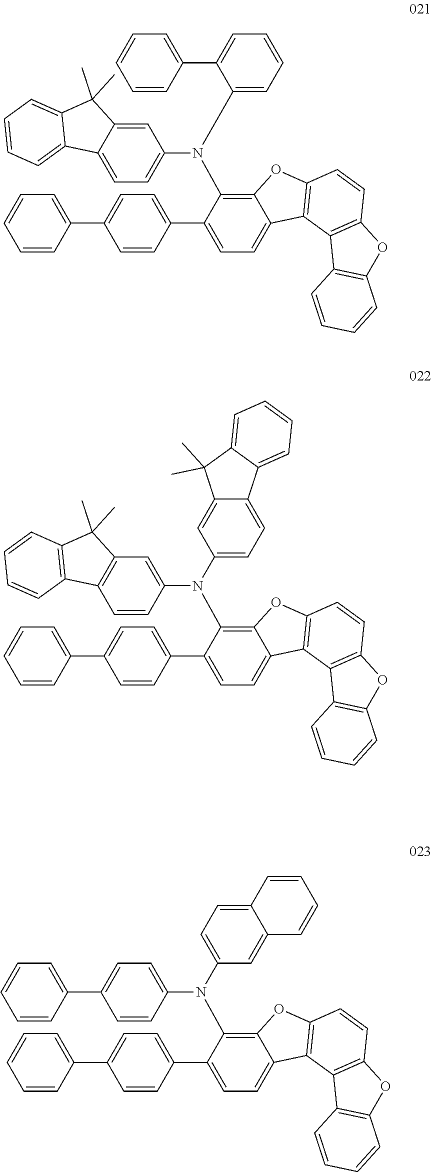 [SMILES: CC1(C)c2ccccc2-c2ccc(N(c3ccccc3-c3ccccc3)c3c(-c4ccc(-c5ccccc5)cc4)ccc4c3oc3ccc5oc6ccccc6c5c34)cc21 ; CC1(C)c2ccccc2-c2ccc(N(c3ccc4c(c3)C(C)(C)c3ccccc3-4)c3c(-c4ccc(-c5ccccc5)cc4)ccc4c3oc3ccc5oc6ccccc6c5c34)cc21 ; c1ccc(-c2ccc(-c3ccc4c(oc5ccc6oc7ccccc7c6c54)c3N(c3ccc(-c4ccccc4)cc3)c3ccc4ccccc4c3)cc2)cc1]

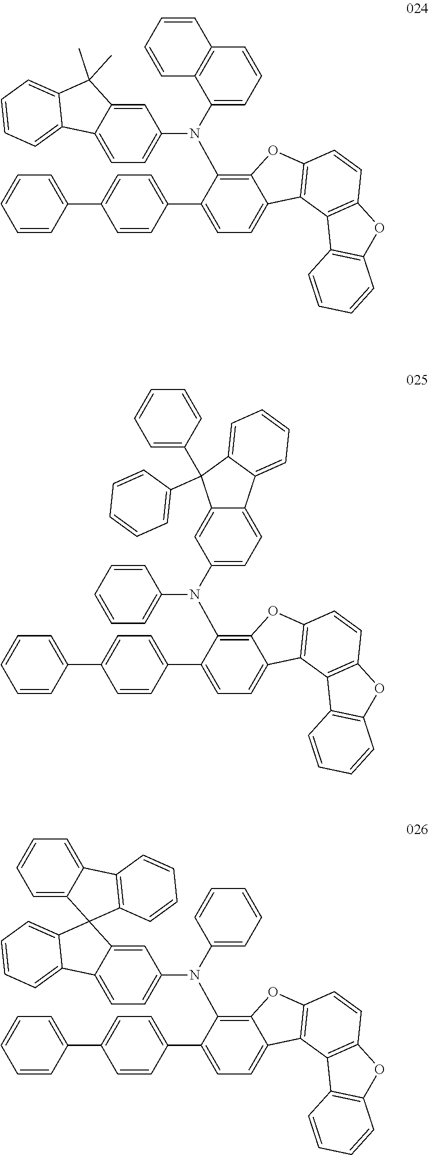 [SMILES: CC1(C)c2ccccc2-c2ccc(N(c3cccc4ccccc34)c3c(-c4ccc(-c5ccccc5)cc4)ccc4c3oc3ccc5oc6ccccc6c5c34)cc21 ; c1ccc(-c2ccc(-c3ccc4c(oc5ccc6oc7ccccc7c6c54)c3N(c3ccccc3)c3ccc4c(c3)C(c3ccccc3)(c3ccccc3)c3ccccc3-4)cc2)cc1 ; c1ccc(-c2ccc(-c3ccc4c(oc5ccc6oc7ccccc7c6c54)c3N(c3ccccc3)c3ccc4c(c3)C3(c5ccccc5-c5ccccc53)c3ccccc3-4)cc2)cc1]

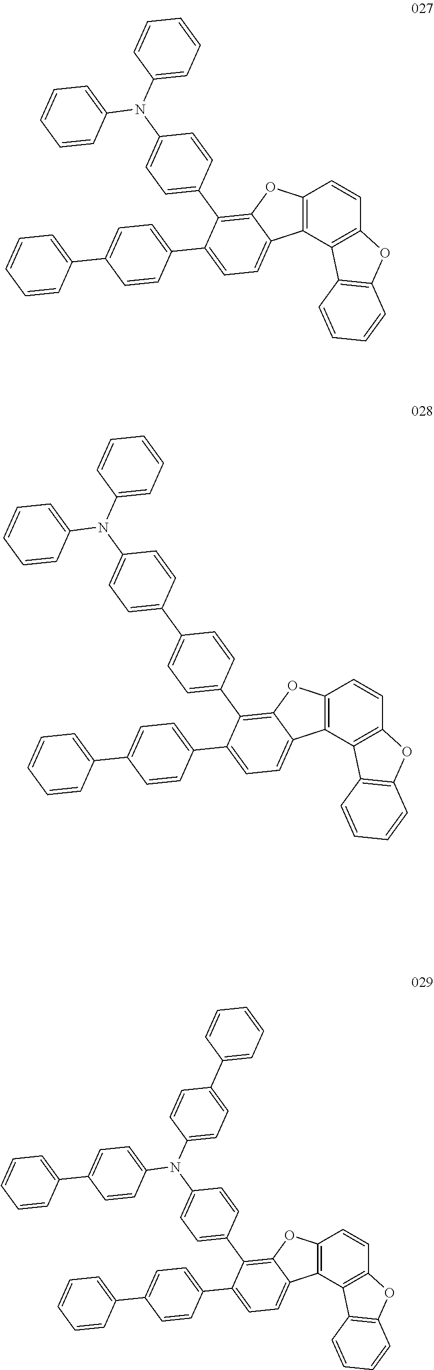 [SMILES: c1ccc(-c2ccc(-c3ccc4c(oc5ccc6oc7ccccc7c6c54)c3-c3ccc(N(c4ccccc4)c4ccccc4)cc3)cc2)cc1 ; c1ccc(-c2ccc(-c3ccc4c(oc5ccc6oc7ccccc7c6c54)c3-c3ccc(-c4ccc(N(c5ccccc5)c5ccccc5)cc4)cc3)cc2)cc1 ; c1ccc(-c2ccc(-c3ccc4c(oc5ccc6oc7ccccc7c6c54)c3-c3ccc(N(c4ccc(-c5ccccc5)cc4)c4ccc(-c5ccccc5)cc4)cc3)cc2)cc1]

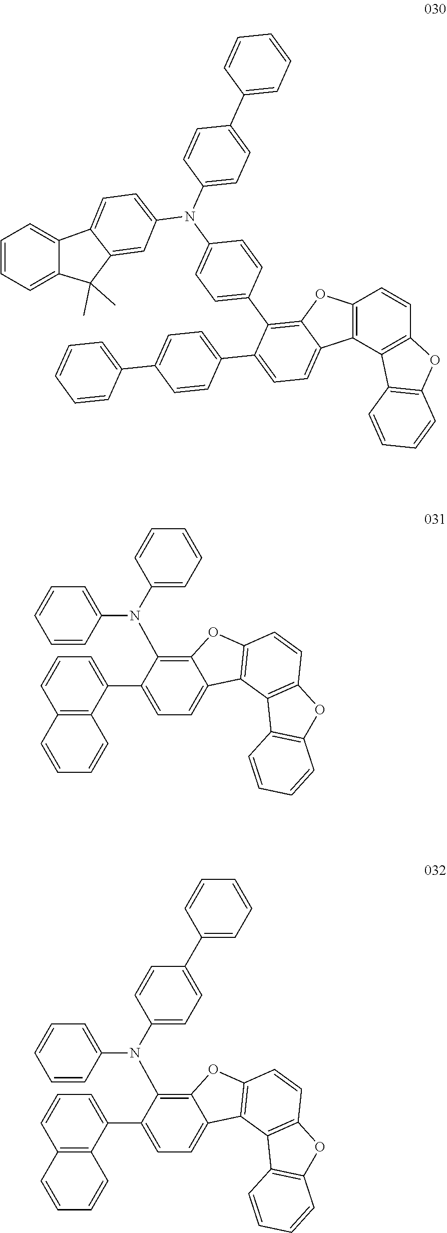 [SMILES: CC1(C)c2ccccc2-c2ccc(N(c3ccc(-c4ccccc4)cc3)c3ccc(-c4c(-c5ccc(-c6ccccc6)cc5)ccc5c4oc4ccc6oc7ccccc7c6c45)cc3)cc21 ; c1ccc(N(c2ccccc2)c2c(-c3cccc4ccccc34)ccc3c2oc2ccc4oc5ccccc5c4c23)cc1 ; c1ccc(-c2ccc(N(c3ccccc3)c3c(-c4cccc5ccccc45)ccc4c3oc3ccc5oc6ccccc6c5c34)cc2)cc1]

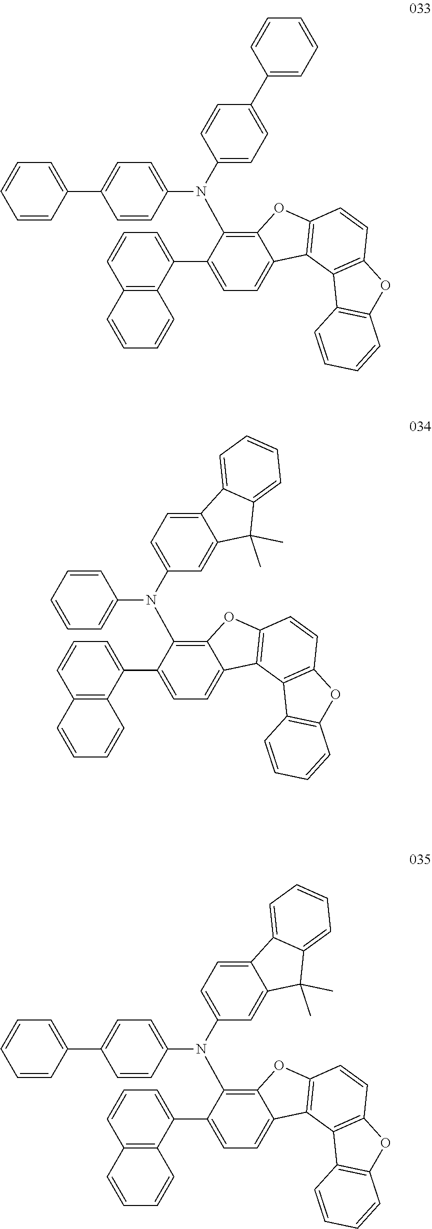 [SMILES: c1ccc(-c2ccc(N(c3ccc(-c4ccccc4)cc3)c3c(-c4cccc5ccccc45)ccc4c3oc3ccc5oc6ccccc6c5c34)cc2)cc1 ; CC1(C)c2ccccc2-c2ccc(N(c3ccccc3)c3c(-c4cccc5ccccc45)ccc4c3oc3ccc5oc6ccccc6c5c34)cc21 ; CC1(C)c2ccccc2-c2ccc(N(c3ccc(-c4ccccc4)cc3)c3c(-c4cccc5ccccc45)ccc4c3oc3ccc5oc6ccccc6c5c34)cc21]

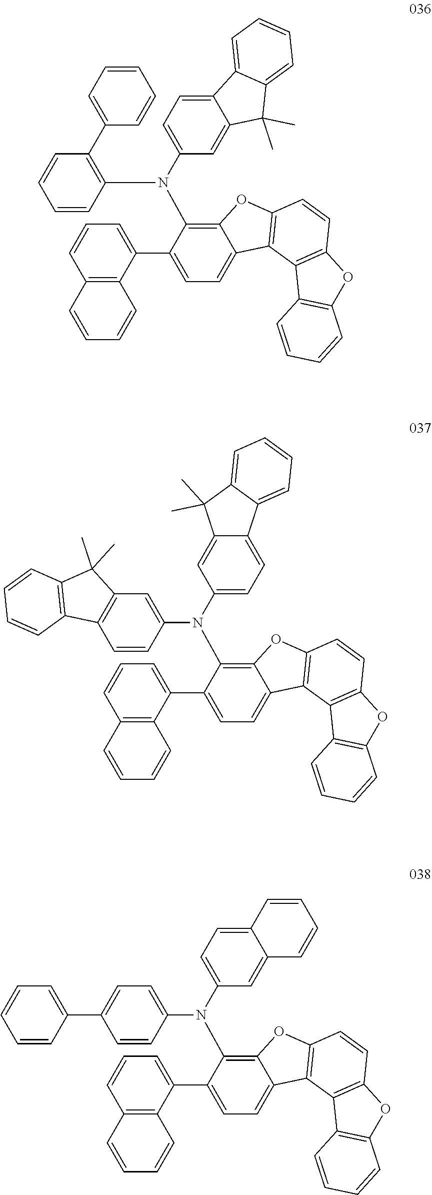 [SMILES: CC1(C)c2ccccc2-c2ccc(N(c3ccccc3-c3ccccc3)c3c(-c4cccc5ccccc45)ccc4c3oc3ccc5oc6ccccc6c5c34)cc21 ; CC1(C)c2ccccc2-c2ccc(N(c3ccc4c(c3)C(C)(C)c3ccccc3-4)c3c(-c4cccc5ccccc45)ccc4c3oc3ccc5oc6ccccc6c5c34)cc21 ; c1ccc(-c2ccc(N(c3ccc4ccccc4c3)c3c(-c4cccc5ccccc45)ccc4c3oc3ccc5oc6ccccc6c5c34)cc2)cc1]

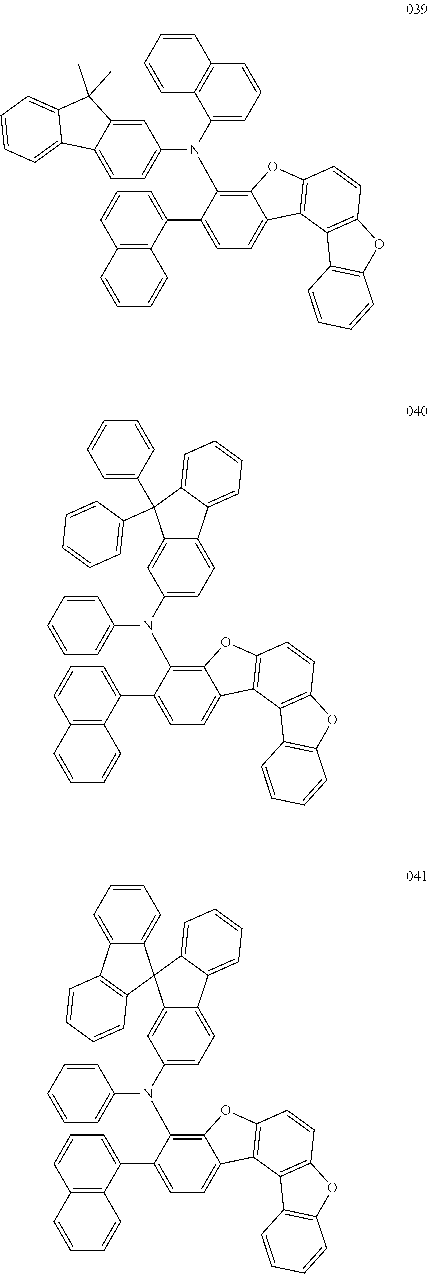 [SMILES: CC1(C)c2ccccc2-c2ccc(N(c3cccc4ccccc34)c3c(-c4cccc5ccccc45)ccc4c3oc3ccc5oc6ccccc6c5c34)cc21 ; c1ccc(N(c2ccc3c(c2)C(c2ccccc2)(c2ccccc2)c2ccccc2-3)c2c(-c3cccc4ccccc34)ccc3c2oc2ccc4oc5ccccc5c4c23)cc1 ; c1ccc(N(c2ccc3c(c2)C2(c4ccccc4-c4ccccc42)c2ccccc2-3)c2c(-c3cccc4ccccc34)ccc3c2oc2ccc4oc5ccccc5c4c23)cc1]

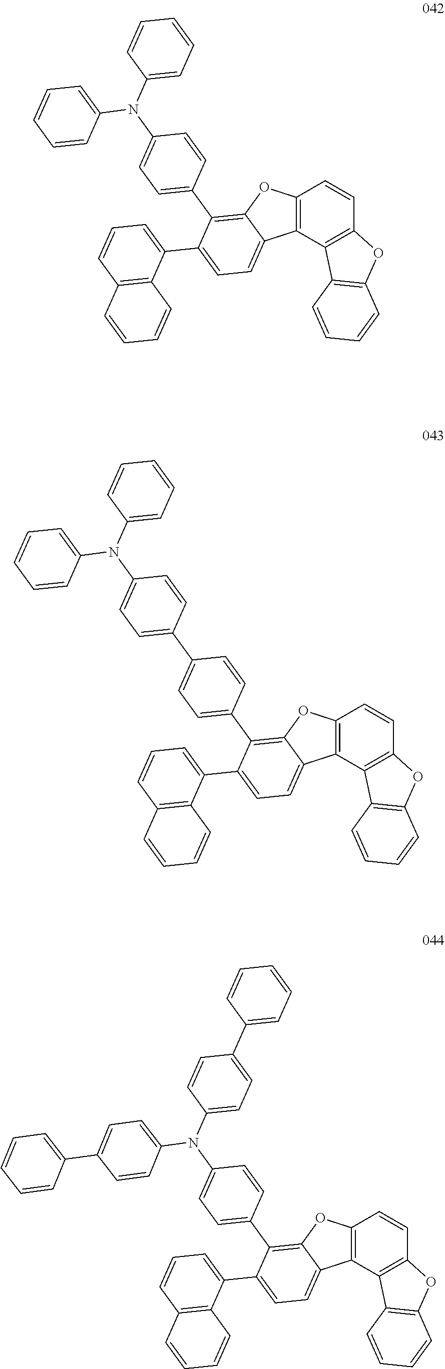 [SMILES: c1ccc(N(c2ccccc2)c2ccc(-c3c(-c4cccc5ccccc45)ccc4c3oc3ccc5oc6ccccc6c5c34)cc2)cc1 ; c1ccc(N(c2ccccc2)c2ccc(-c3ccc(-c4c(-c5cccc6ccccc56)ccc5c4oc4ccc6oc7ccccc7c6c45)cc3)cc2)cc1 ; c1ccc(-c2ccc(N(c3ccc(-c4ccccc4)cc3)c3ccc(-c4c(-c5cccc6ccccc56)ccc5c4oc4ccc6oc7ccccc7c6c45)cc3)cc2)cc1]

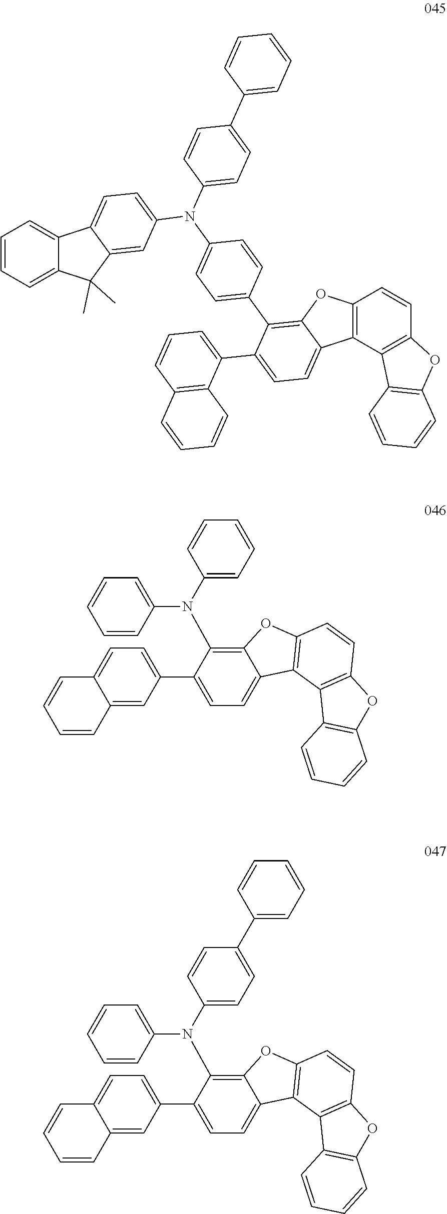 [SMILES: CC1(C)c2ccccc2-c2ccc(N(c3ccc(-c4ccccc4)cc3)c3ccc(-c4c(-c5cccc6ccccc56)ccc5c4oc4ccc6oc7ccccc7c6c45)cc3)cc21 ; c1ccc(N(c2ccccc2)c2c(-c3ccc4ccccc4c3)ccc3c2oc2ccc4oc5ccccc5c4c23)cc1 ; c1ccc(-c2ccc(N(c3ccccc3)c3c(-c4ccc5ccccc5c4)ccc4c3oc3ccc5oc6ccccc6c5c34)cc2)cc1]

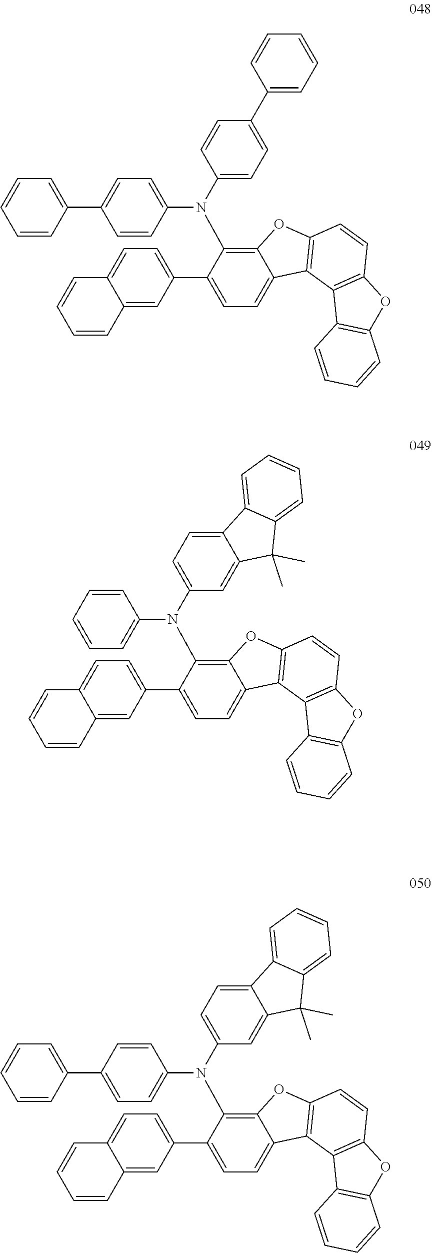 [SMILES: c1ccc(-c2ccc(N(c3ccc(-c4ccccc4)cc3)c3c(-c4ccc5ccccc5c4)ccc4c3oc3ccc5oc6ccccc6c5c34)cc2)cc1 ; CC1(C)c2ccccc2-c2ccc(N(c3ccccc3)c3c(-c4ccc5ccccc5c4)ccc4c3oc3ccc5oc6ccccc6c5c34)cc21 ; CC1(C)c2ccccc2-c2ccc(N(c3ccc(-c4ccccc4)cc3)c3c(-c4ccc5ccccc5c4)ccc4c3oc3ccc5oc6ccccc6c5c34)cc21]

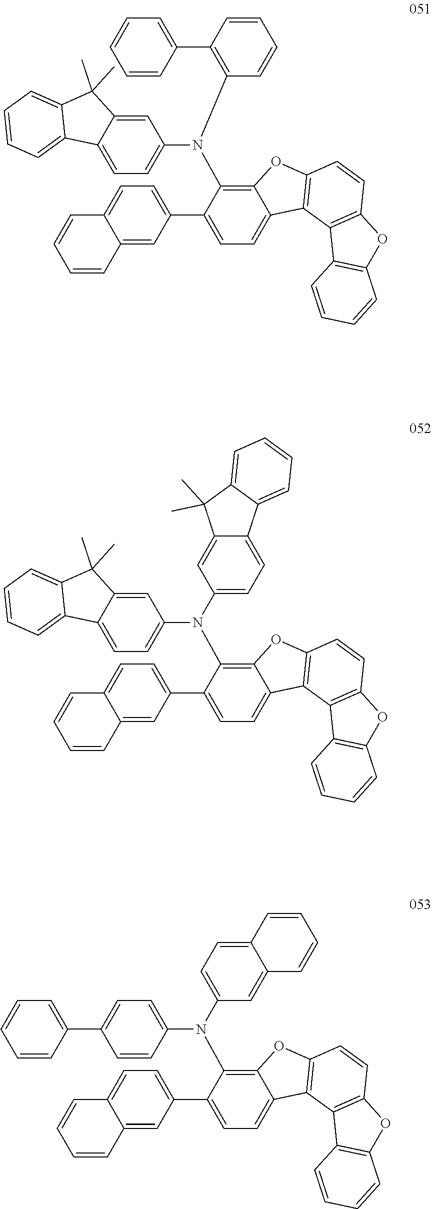 [SMILES: CC1(C)c2ccccc2-c2ccc(N(c3ccccc3-c3ccccc3)c3c(-c4ccc5ccccc5c4)ccc4c3oc3ccc5oc6ccccc6c5c34)cc21 ; CC1(C)c2ccccc2-c2ccc(N(c3ccc4c(c3)C(C)(C)c3ccccc3-4)c3c(-c4ccc5ccccc5c4)ccc4c3oc3ccc5oc6ccccc6c5c34)cc21 ; c1ccc(-c2ccc(N(c3ccc4ccccc4c3)c3c(-c4ccc5ccccc5c4)ccc4c3oc3ccc5oc6ccccc6c5c34)cc2)cc1]

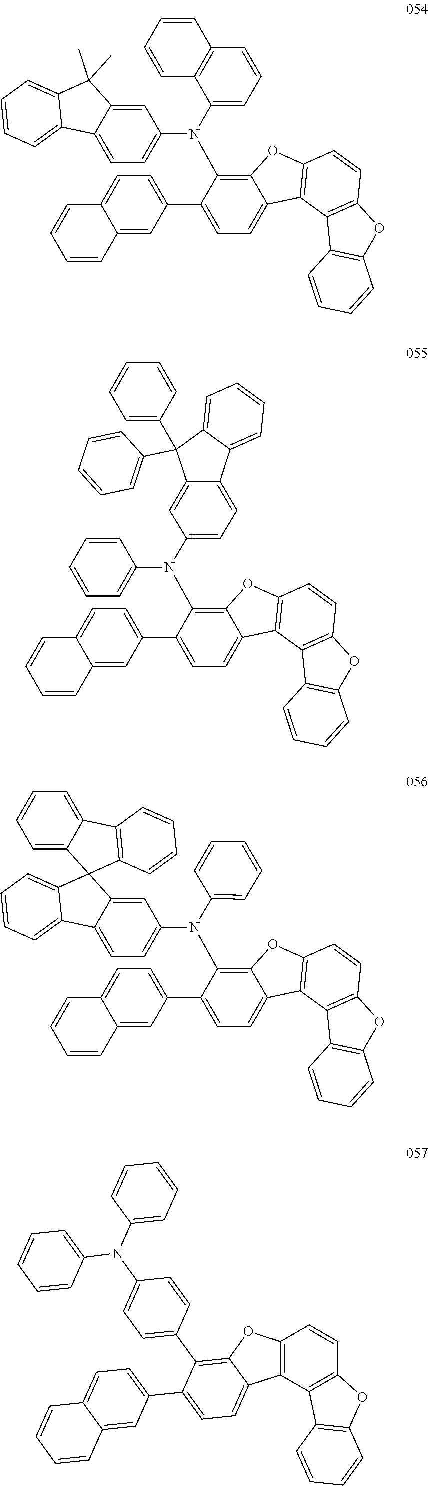 [SMILES: CC1(C)c2ccccc2-c2ccc(N(c3cccc4ccccc34)c3c(-c4ccc5ccccc5c4)ccc4c3oc3ccc5oc6ccccc6c5c34)cc21 ; c1ccc(N(c2ccc3c(c2)C(c2ccccc2)(c2ccccc2)c2ccccc2-3)c2c(-c3ccc4ccccc4c3)ccc3c2oc2ccc4oc5ccccc5c4c23)cc1 ; c1ccc(N(c2ccc3c(c2)C2(c4ccccc4-c4ccccc42)c2ccccc2-3)c2c(-c3ccc4ccccc4c3)ccc3c2oc2ccc4oc5ccccc5c4c23)cc1 ; c1ccc(N(c2ccccc2)c2ccc(-c3c(-c4ccc5ccccc5c4)ccc4c3oc3ccc5oc6ccccc6c5c34)cc2)cc1]

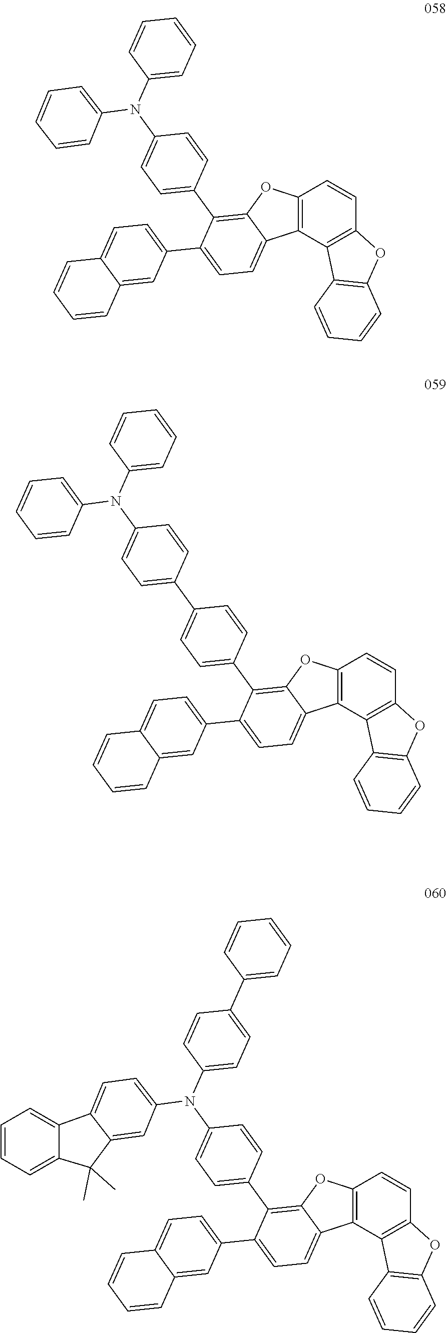 [SMILES: c1ccc(N(c2ccccc2)c2ccc(-c3c(-c4ccc5ccccc5c4)ccc4c3oc3ccc5oc6ccccc6c5c34)cc2)cc1 ; c1ccc(N(c2ccccc2)c2ccc(-c3ccc(-c4c(-c5ccc6ccccc6c5)ccc5c4oc4ccc6oc7ccccc7c6c45)cc3)cc2)cc1 ; CC1(C)c2ccccc2-c2ccc(N(c3ccc(-c4ccccc4)cc3)c3ccc(-c4c(-c5ccc6ccccc6c5)ccc5c4oc4ccc6oc7ccccc7c6c45)cc3)cc21]

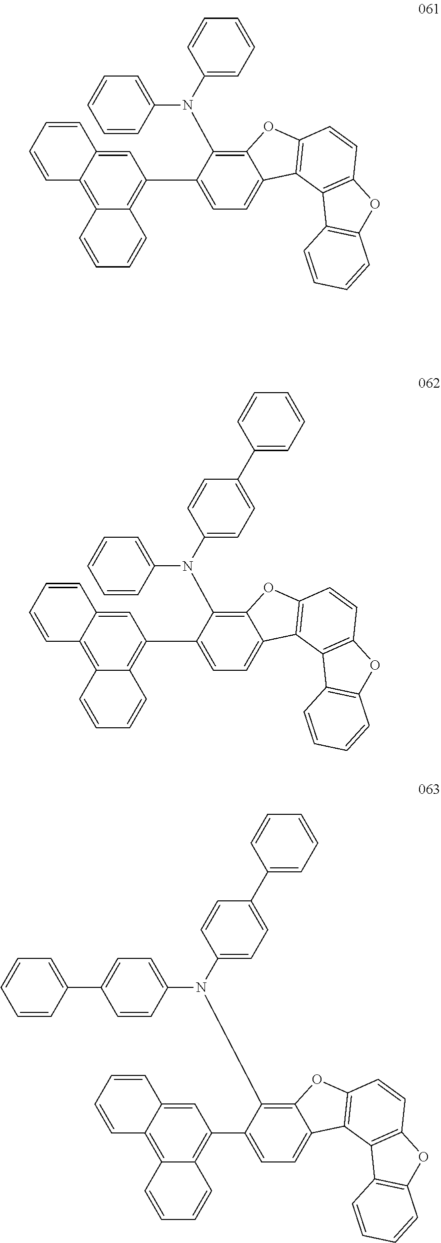 [SMILES: c1ccc(N(c2ccccc2)c2c(-c3cc4ccccc4c4ccccc34)ccc3c2oc2ccc4oc5ccccc5c4c23)cc1 ; c1ccc(-c2ccc(N(c3ccccc3)c3c(-c4cc5ccccc5c5ccccc45)ccc4c3oc3ccc5oc6ccccc6c5c34)cc2)cc1 ; c1ccc(-c2ccc(N(c3ccc(-c4ccccc4)cc3)c3c(-c4cc5ccccc5c5ccccc45)ccc4c3oc3ccc5oc6ccccc6c5c34)cc2)cc1]

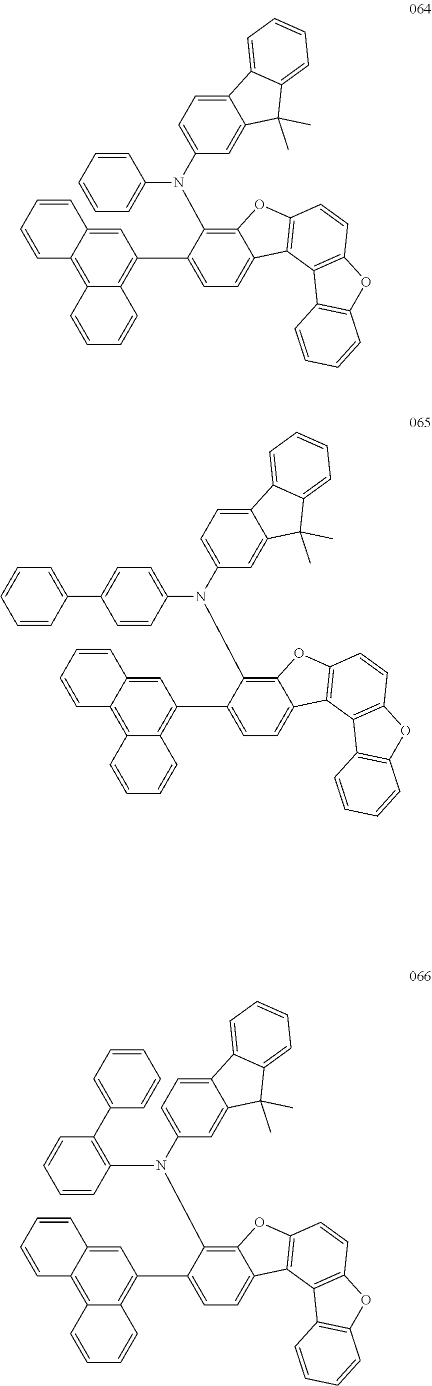 [SMILES: CC1(C)c2ccccc2-c2ccc(N(c3ccccc3)c3c(-c4cc5ccccc5c5ccccc45)ccc4c3oc3ccc5oc6ccccc6c5c34)cc21 ; CC1(C)c2ccccc2-c2ccc(N(c3ccc(-c4ccccc4)cc3)c3c(-c4cc5ccccc5c5ccccc45)ccc4c3oc3ccc5oc6ccccc6c5c34)cc21 ; CC1(C)c2ccccc2-c2ccc(N(c3ccccc3-c3ccccc3)c3c(-c4cc5ccccc5c5ccccc45)ccc4c3oc3ccc5oc6ccccc6c5c34)cc21]

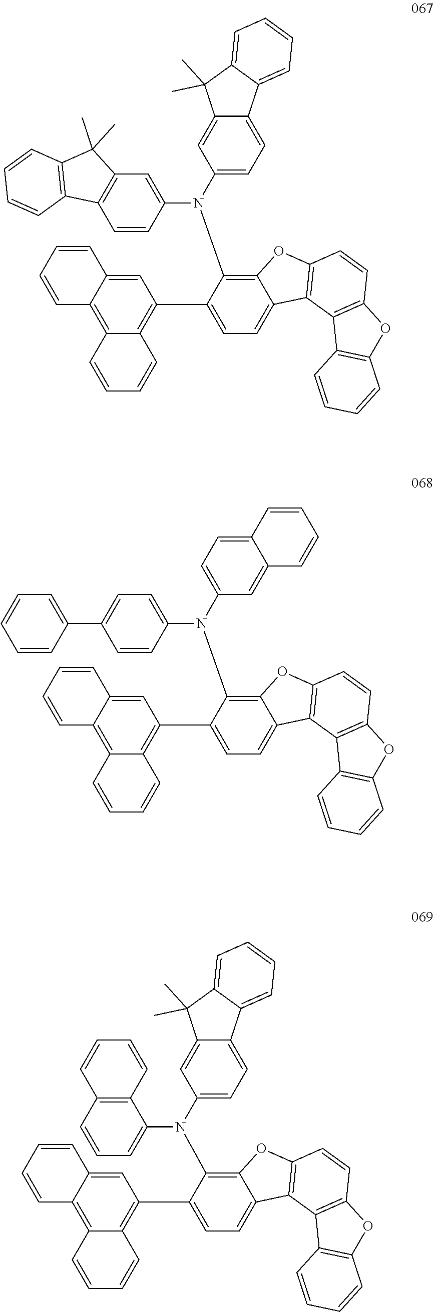 [SMILES: CC1(C)c2ccccc2-c2ccc(N(c3ccc4c(c3)C(C)(C)c3ccccc3-4)c3c(-c4cc5ccccc5c5ccccc45)ccc4c3oc3ccc5oc6ccccc6c5c34)cc21 ; c1ccc(-c2ccc(N(c3ccc4ccccc4c3)c3c(-c4cc5ccccc5c5ccccc45)ccc4c3oc3ccc5oc6ccccc6c5c34)cc2)cc1 ; CC1(C)c2ccccc2-c2ccc(N(c3cccc4ccccc34)c3c(-c4cc5ccccc5c5ccccc45)ccc4c3oc3ccc5oc6ccccc6c5c34)cc21]

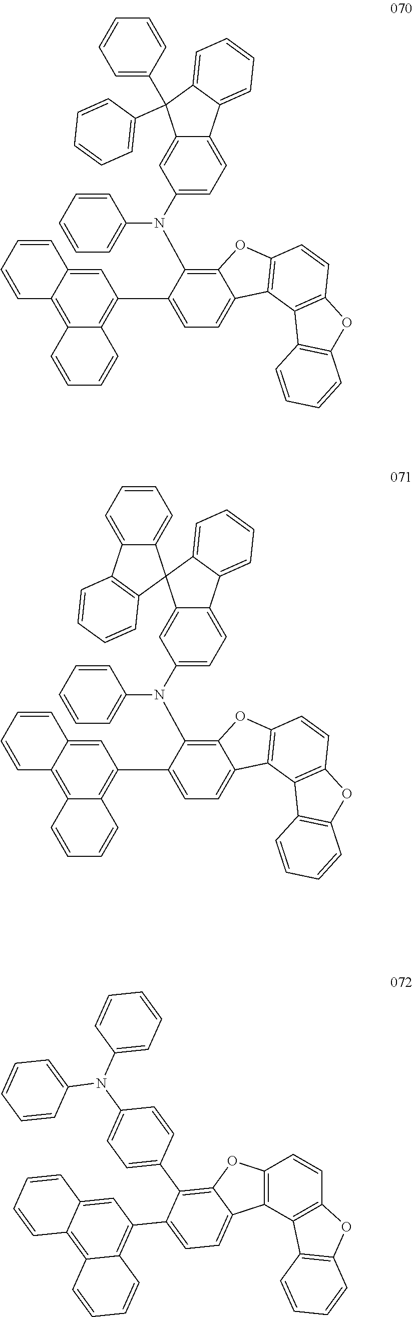 [SMILES: c1ccc(N(c2ccc3c(c2)C(c2ccccc2)(c2ccccc2)c2ccccc2-3)c2c(-c3cc4ccccc4c4ccccc34)ccc3c2oc2ccc4oc5ccccc5c4c23)cc1 ; c1ccc(N(c2ccc3c(c2)C2(c4ccccc4-c4ccccc42)c2ccccc2-3)c2c(-c3cc4ccccc4c4ccccc34)ccc3c2oc2ccc4oc5ccccc5c4c23)cc1 ; c1ccc(N(c2ccccc2)c2ccc(-c3c(-c4cc5ccccc5c5ccccc45)ccc4c3oc3ccc5oc6ccccc6c5c34)cc2)cc1]

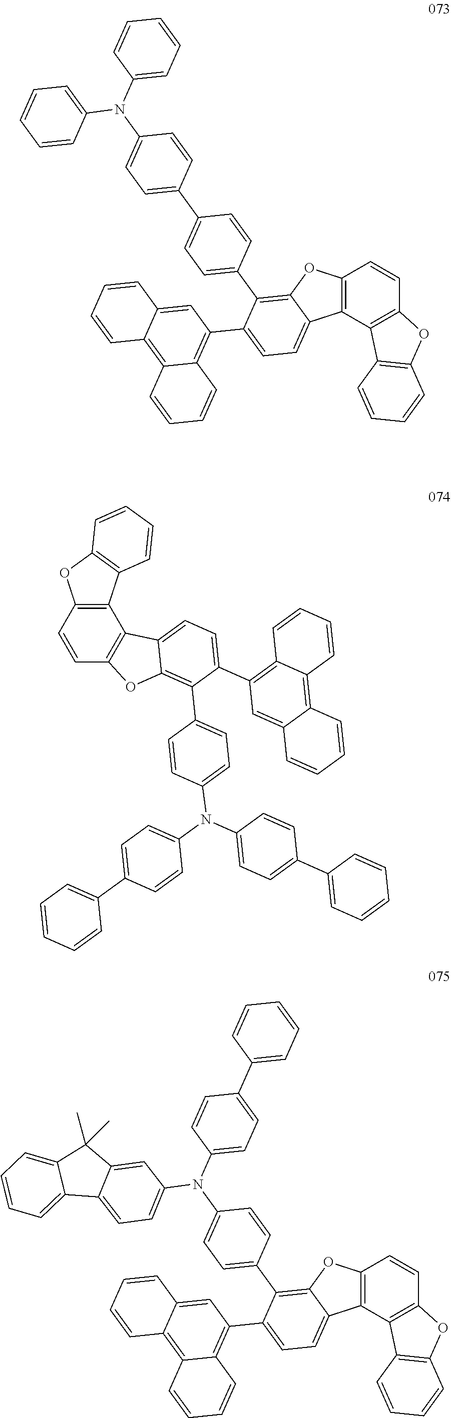 [SMILES: c1ccc(N(c2ccccc2)c2ccc(-c3ccc(-c4c(-c5cc6ccccc6c6ccccc56)ccc5c4oc4ccc6oc7ccccc7c6c45)cc3)cc2)cc1 ; c1ccc(-c2ccc(N(c3ccc(-c4ccccc4)cc3)c3ccc(-c4c(-c5cc6ccccc6c6ccccc56)ccc5c4oc4ccc6oc7ccccc7c6c45)cc3)cc2)cc1 ; CC1(C)c2ccccc2-c2ccc(N(c3ccc(-c4ccccc4)cc3)c3ccc(-c4c(-c5cc6ccccc6c6ccccc56)ccc5c4oc4ccc6oc7ccccc7c6c45)cc3)cc21]

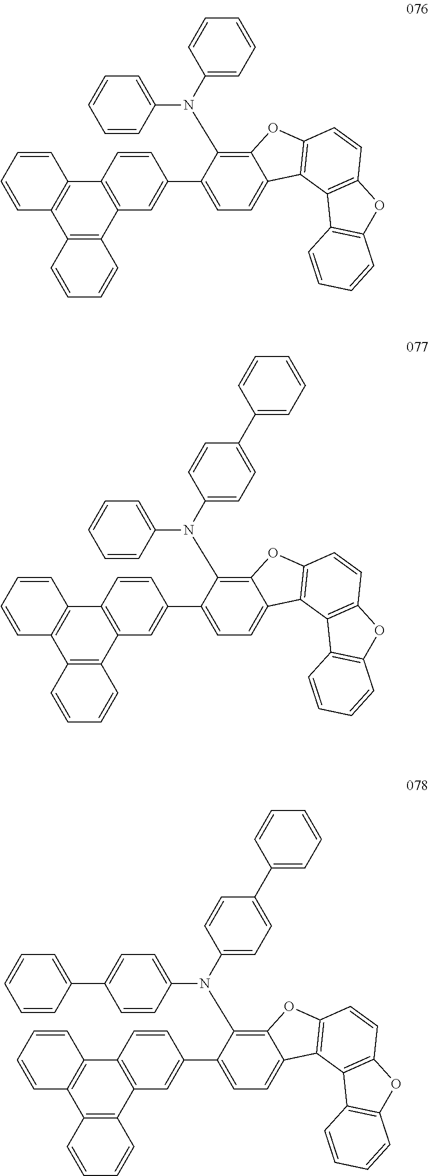 [SMILES: c1ccc(N(c2ccccc2)c2c(-c3ccc4c5ccccc5c5ccccc5c4c3)ccc3c2oc2ccc4oc5ccccc5c4c23)cc1 ; c1ccc(-c2ccc(N(c3ccccc3)c3c(-c4ccc5c6ccccc6c6ccccc6c5c4)ccc4c3oc3ccc5oc6ccccc6c5c34)cc2)cc1 ; c1ccc(-c2ccc(N(c3ccc(-c4ccccc4)cc3)c3c(-c4ccc5c6ccccc6c6ccccc6c5c4)ccc4c3oc3ccc5oc6ccccc6c5c34)cc2)cc1]

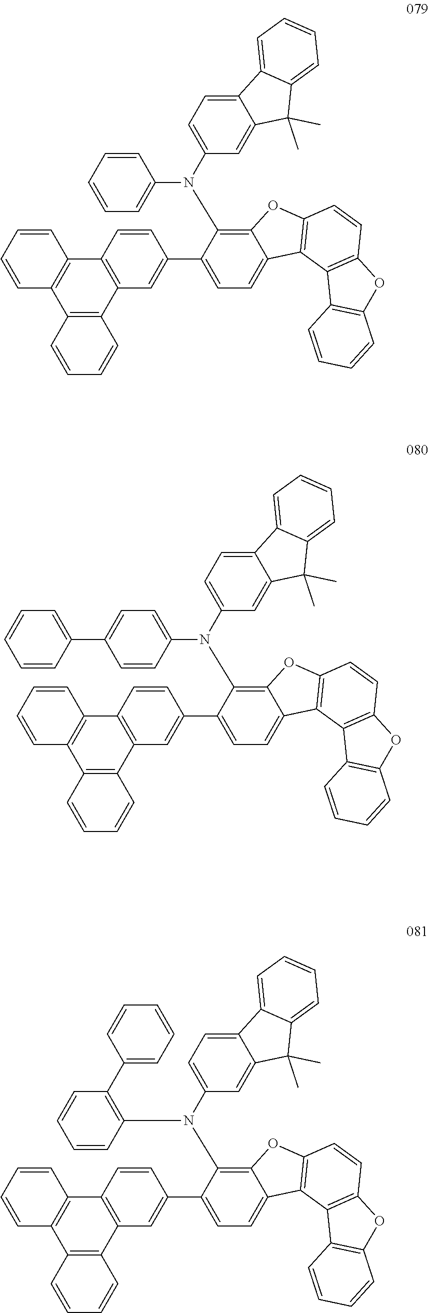 [SMILES: CC1(C)c2ccccc2-c2ccc(N(c3ccccc3)c3c(-c4ccc5c6ccccc6c6ccccc6c5c4)ccc4c3oc3ccc5oc6ccccc6c5c34)cc21 ; CC1(C)c2ccccc2-c2ccc(N(c3ccc(-c4ccccc4)cc3)c3c(-c4ccc5c6ccccc6c6ccccc6c5c4)ccc4c3oc3ccc5oc6ccccc6c5c34)cc21 ; CC1(C)c2ccccc2-c2ccc(N(c3ccccc3-c3ccccc3)c3c(-c4ccc5c6ccccc6c6ccccc6c5c4)ccc4c3oc3ccc5oc6ccccc6c5c34)cc21]

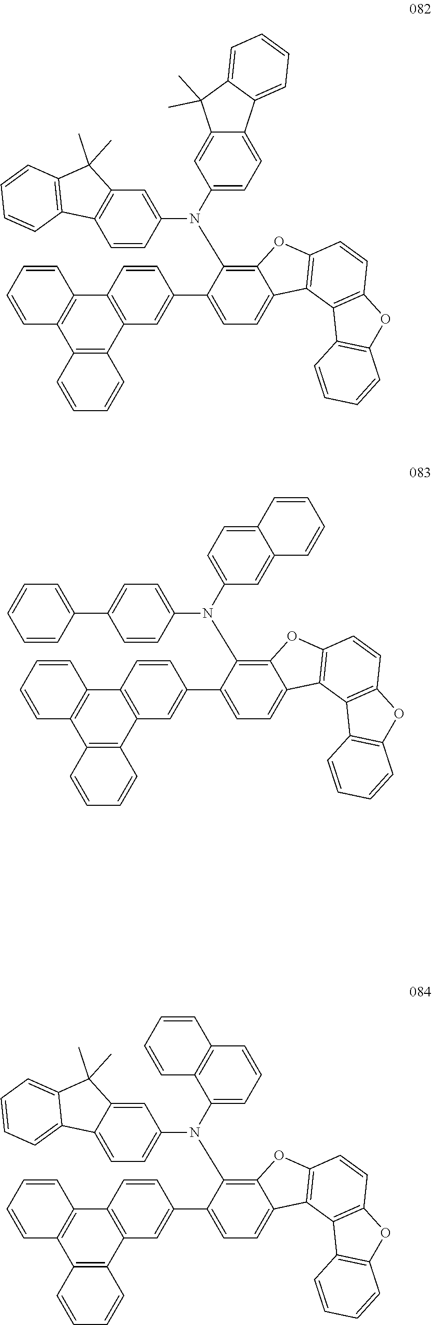 [SMILES: CC1(C)c2ccccc2-c2ccc(N(c3ccc4c(c3)C(C)(C)c3ccccc3-4)c3c(-c4ccc5c6ccccc6c6ccccc6c5c4)ccc4c3oc3ccc5oc6ccccc6c5c34)cc21 ; c1ccc(-c2ccc(N(c3ccc4ccccc4c3)c3c(-c4ccc5c6ccccc6c6ccccc6c5c4)ccc4c3oc3ccc5oc6ccccc6c5c34)cc2)cc1 ; CC1(C)c2ccccc2-c2ccc(N(c3cccc4ccccc34)c3c(-c4ccc5c6ccccc6c6ccccc6c5c4)ccc4c3oc3ccc5oc6ccccc6c5c34)cc21]

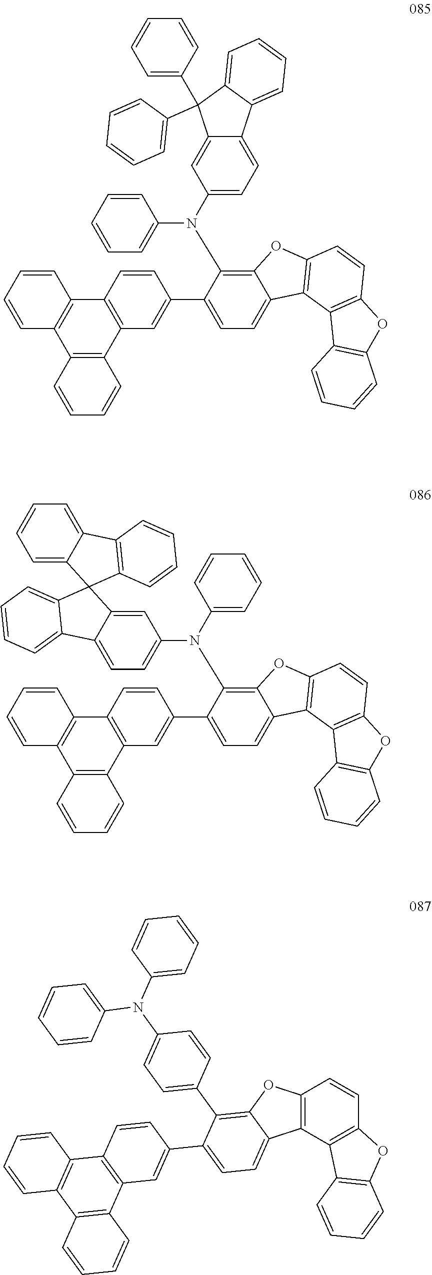 [SMILES: c1ccc(N(c2ccc3c(c2)C(c2ccccc2)(c2ccccc2)c2ccccc2-3)c2c(-c3ccc4c5ccccc5c5ccccc5c4c3)ccc3c2oc2ccc4oc5ccccc5c4c23)cc1 ; c1ccc(N(c2ccc3c(c2)C2(c4ccccc4-c4ccccc42)c2ccccc2-3)c2c(-c3ccc4c5ccccc5c5ccccc5c4c3)ccc3c2oc2ccc4oc5ccccc5c4c23)cc1 ; c1ccc(N(c2ccccc2)c2ccc(-c3c(-c4ccc5c6ccccc6c6ccccc6c5c4)ccc4c3oc3ccc5oc6ccccc6c5c34)cc2)cc1]

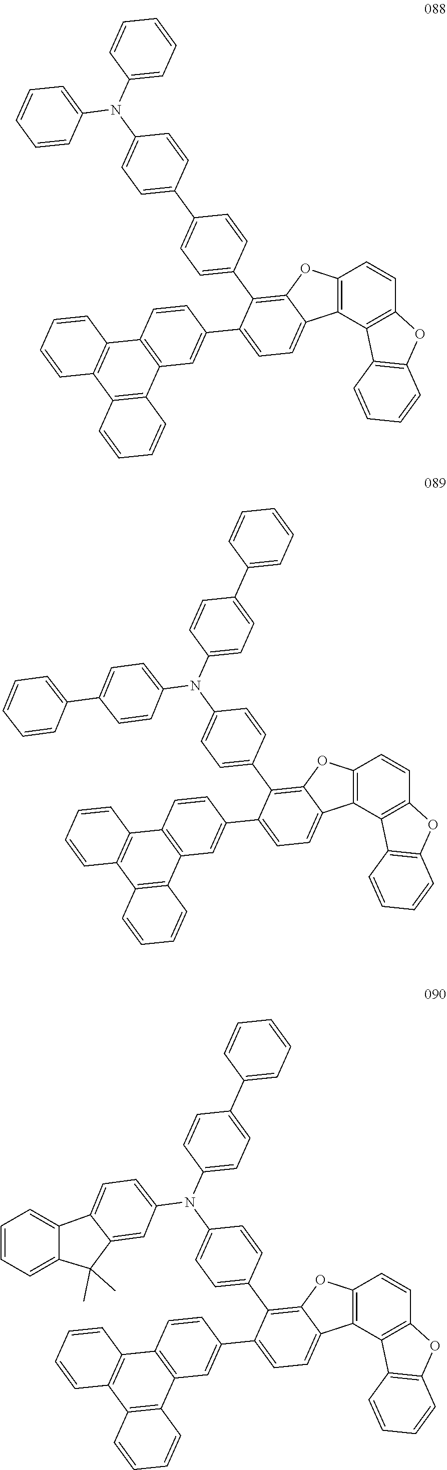 [SMILES: c1ccc(N(c2ccccc2)c2ccc(-c3ccc(-c4c(-c5ccc6c7ccccc7c7ccccc7c6c5)ccc5c4oc4ccc6oc7ccccc7c6c45)cc3)cc2)cc1 ; c1ccc(-c2ccc(N(c3ccc(-c4ccccc4)cc3)c3ccc(-c4c(-c5ccc6c7ccccc7c7ccccc7c6c5)ccc5c4oc4ccc6oc7ccccc7c6c45)cc3)cc2)cc1 ; CC1(C)c2ccccc2-c2ccc(N(c3ccc(-c4ccccc4)cc3)c3ccc(-c4c(-c5ccc6c7ccccc7c7ccccc7c6c5)ccc5c4oc4ccc6oc7ccccc7c6c45)cc3)cc21]

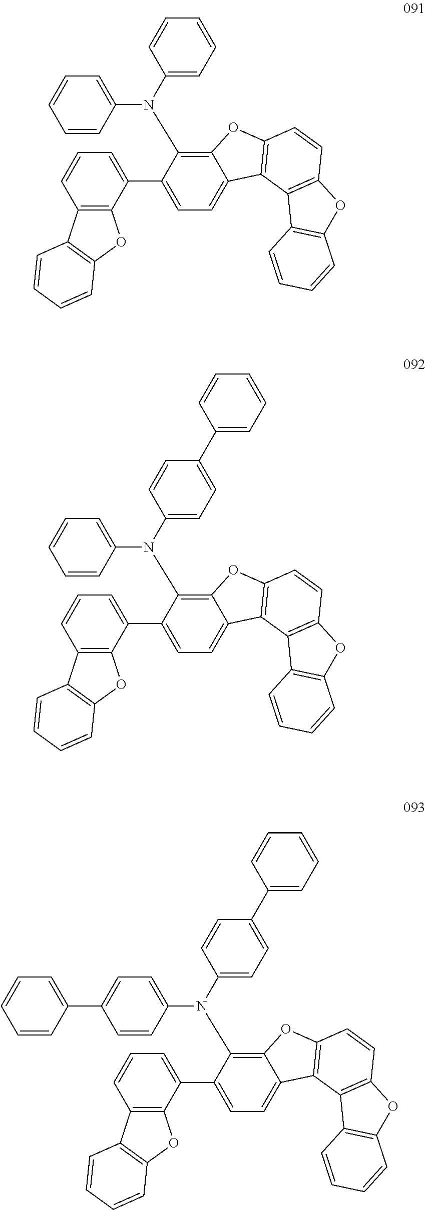 [SMILES: c1ccc(N(c2ccccc2)c2c(-c3cccc4c3oc3ccccc34)ccc3c2oc2ccc4oc5ccccc5c4c23)cc1 ; c1ccc(-c2ccc(N(c3ccccc3)c3c(-c4cccc5c4oc4ccccc45)ccc4c3oc3ccc5oc6ccccc6c5c34)cc2)cc1 ; c1ccc(-c2ccc(N(c3ccc(-c4ccccc4)cc3)c3c(-c4cccc5c4oc4ccccc45)ccc4c3oc3ccc5oc6ccccc6c5c34)cc2)cc1]

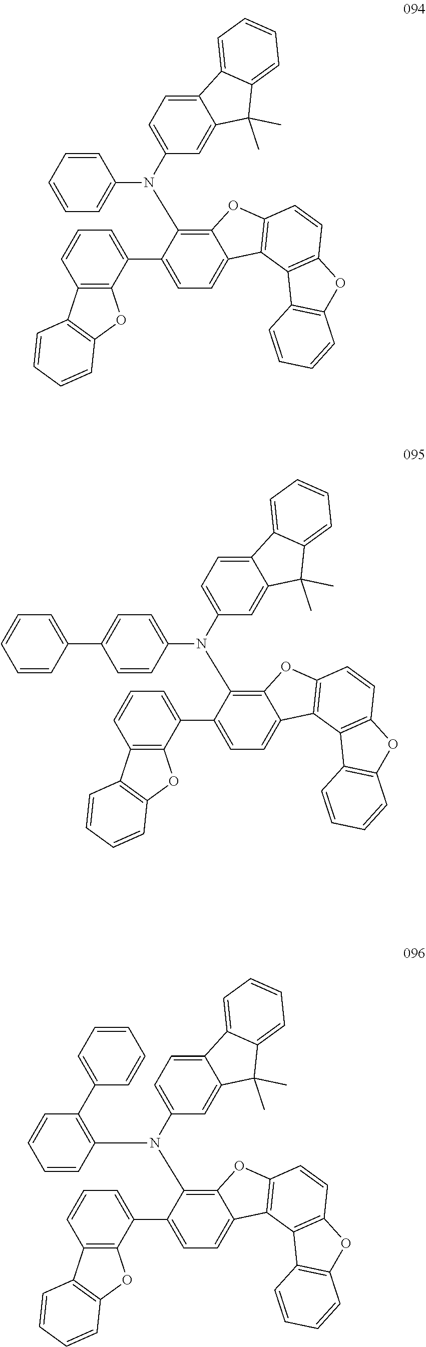 [SMILES: CC1(C)c2ccccc2-c2ccc(N(c3ccccc3)c3c(-c4cccc5c4oc4ccccc45)ccc4c3oc3ccc5oc6ccccc6c5c34)cc21 ; CC1(C)c2ccccc2-c2ccc(N(c3ccc(-c4ccccc4)cc3)c3c(-c4cccc5c4oc4ccccc45)ccc4c3oc3ccc5oc6ccccc6c5c34)cc21 ; CC1(C)c2ccccc2-c2ccc(N(c3ccccc3-c3ccccc3)c3c(-c4cccc5c4oc4ccccc45)ccc4c3oc3ccc5oc6ccccc6c5c34)cc21]

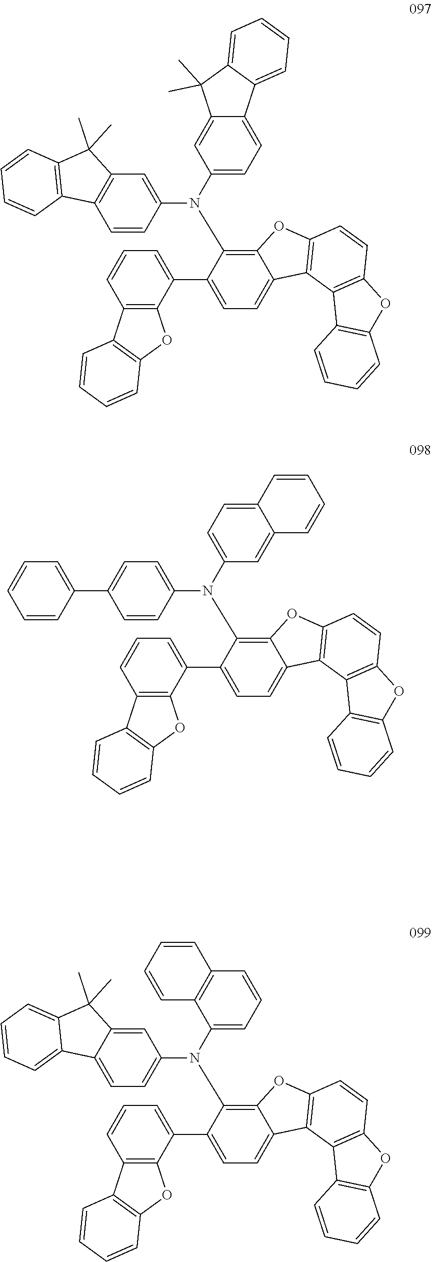 [SMILES: CC1(C)c2ccccc2-c2ccc(N(c3ccc4c(c3)C(C)(C)c3ccccc3-4)c3c(-c4cccc5c4oc4ccccc45)ccc4c3oc3ccc5oc6ccccc6c5c34)cc21 ; c1ccc(-c2ccc(N(c3ccc4ccccc4c3)c3c(-c4cccc5c4oc4ccccc45)ccc4c3oc3ccc5oc6ccccc6c5c34)cc2)cc1 ; CC1(C)c2ccccc2-c2ccc(N(c3cccc4ccccc34)c3c(-c4cccc5c4oc4ccccc45)ccc4c3oc3ccc5oc6ccccc6c5c34)cc21]

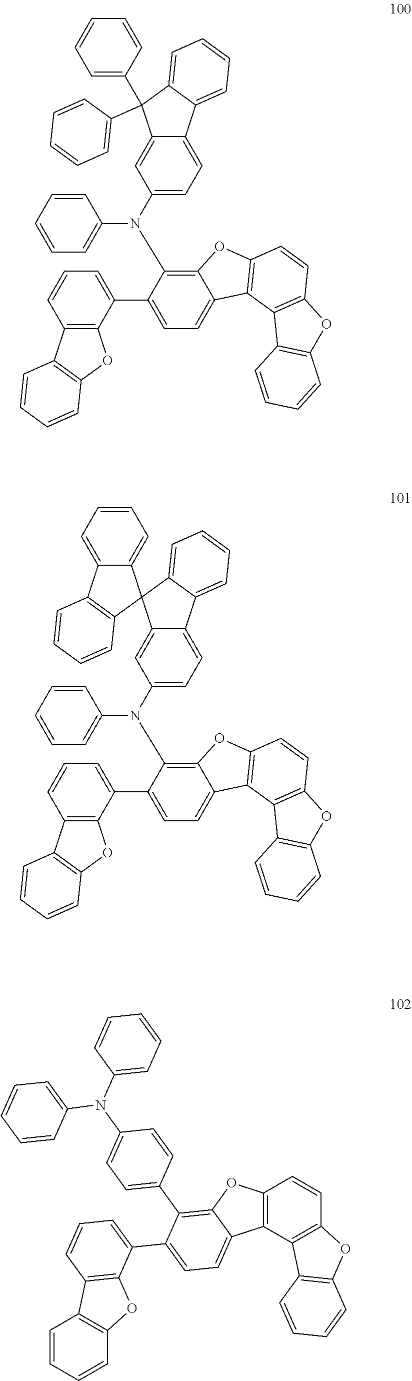 [SMILES: c1ccc(N(c2ccc3c(c2)C(c2ccccc2)(c2ccccc2)c2ccccc2-3)c2c(-c3cccc4c3oc3ccccc34)ccc3c2oc2ccc4oc5ccccc5c4c23)cc1 ; c1ccc(N(c2ccc3c(c2)C2(c4ccccc4-c4ccccc42)c2ccccc2-3)c2c(-c3cccc4c3oc3ccccc34)ccc3c2oc2ccc4oc5ccccc5c4c23)cc1 ; c1ccc(N(c2ccccc2)c2ccc(-c3c(-c4cccc5c4oc4ccccc45)ccc4c3oc3ccc5oc6ccccc6c5c34)cc2)cc1]

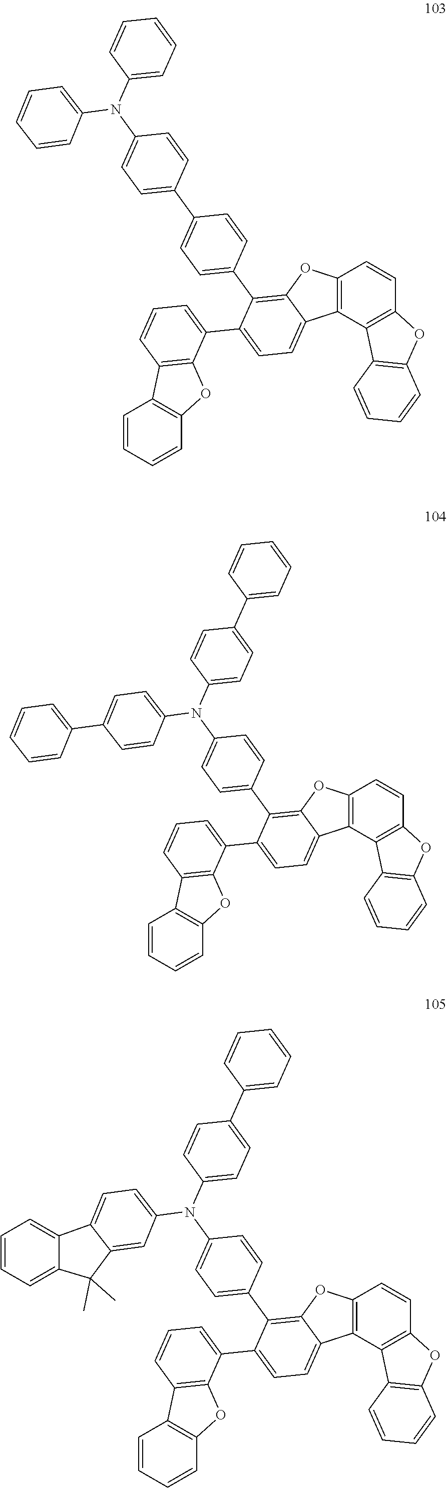 [SMILES: c1ccc(N(c2ccccc2)c2ccc(-c3ccc(-c4c(-c5cccc6c5oc5ccccc56)ccc5c4oc4ccc6oc7ccccc7c6c45)cc3)cc2)cc1 ; c1ccc(-c2ccc(N(c3ccc(-c4ccccc4)cc3)c3ccc(-c4c(-c5cccc6c5oc5ccccc56)ccc5c4oc4ccc6oc7ccccc7c6c45)cc3)cc2)cc1 ; CC1(C)c2ccccc2-c2ccc(N(c3ccc(-c4ccccc4)cc3)c3ccc(-c4c(-c5cccc6c5oc5ccccc56)ccc5c4oc4ccc6oc7ccccc7c6c45)cc3)cc21]

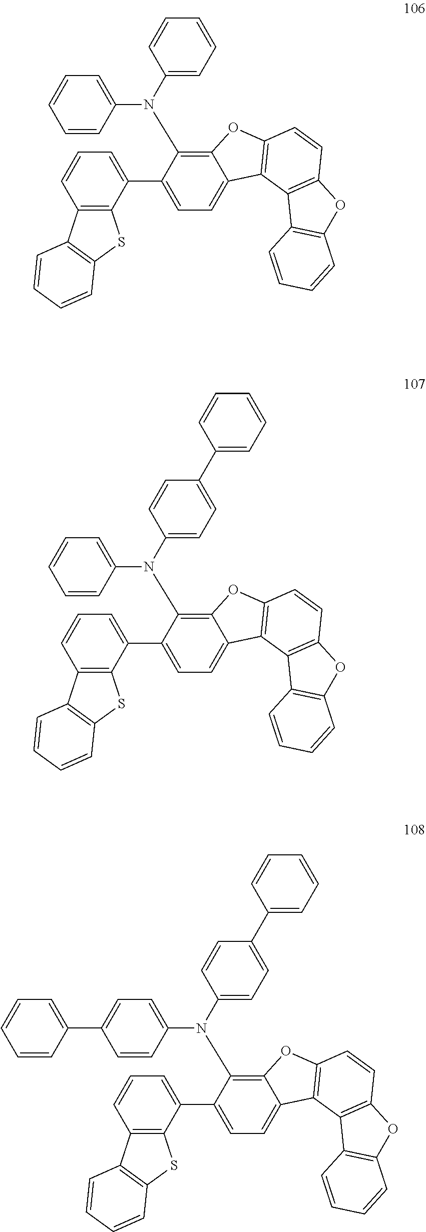 [SMILES: c1ccc(N(c2ccccc2)c2c(-c3cccc4c3sc3ccccc34)ccc3c2oc2ccc4oc5ccccc5c4c23)cc1 ; c1ccc(-c2ccc(N(c3ccccc3)c3c(-c4cccc5c4sc4ccccc45)ccc4c3oc3ccc5oc6ccccc6c5c34)cc2)cc1 ; c1ccc(-c2ccc(N(c3ccc(-c4ccccc4)cc3)c3c(-c4cccc5c4sc4ccccc45)ccc4c3oc3ccc5oc6ccccc6c5c34)cc2)cc1]

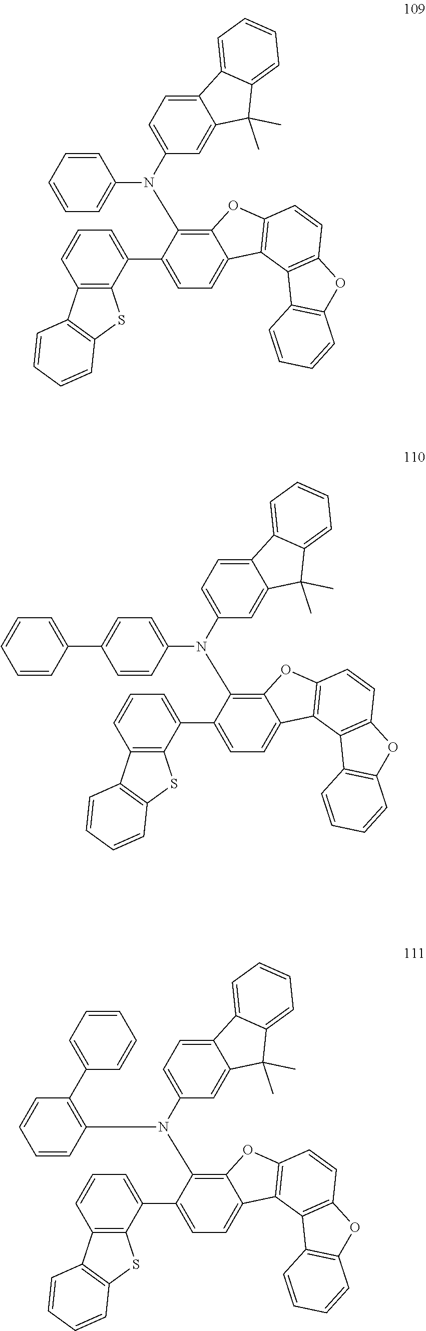 [SMILES: CC1(C)c2ccccc2-c2ccc(N(c3ccccc3)c3c(-c4cccc5c4sc4ccccc45)ccc4c3oc3ccc5oc6ccccc6c5c34)cc21 ; CC1(C)c2ccccc2-c2ccc(N(c3ccc(-c4ccccc4)cc3)c3c(-c4cccc5c4sc4ccccc45)ccc4c3oc3ccc5oc6ccccc6c5c34)cc21 ; CC1(C)c2ccccc2-c2ccc(N(c3ccccc3-c3ccccc3)c3c(-c4cccc5c4sc4ccccc45)ccc4c3oc3ccc5oc6ccccc6c5c34)cc21]

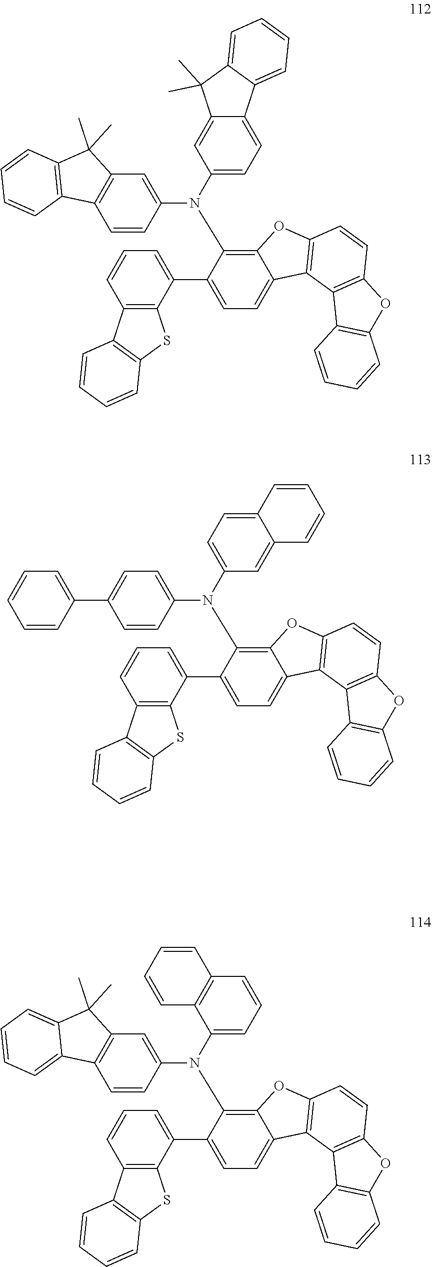 [SMILES: CC1(C)c2ccccc2-c2ccc(N(c3ccc4c(c3)C(C)(C)c3ccccc3-4)c3c(-c4cccc5c4sc4ccccc45)ccc4c3oc3ccc5oc6ccccc6c5c34)cc21 ; c1ccc(-c2ccc(N(c3ccc4ccccc4c3)c3c(-c4cccc5c4sc4ccccc45)ccc4c3oc3ccc5oc6ccccc6c5c34)cc2)cc1 ; CC1(C)c2ccccc2-c2ccc(N(c3cccc4ccccc34)c3c(-c4cccc5c4sc4ccccc45)ccc4c3oc3ccc5oc6ccccc6c5c34)cc21]

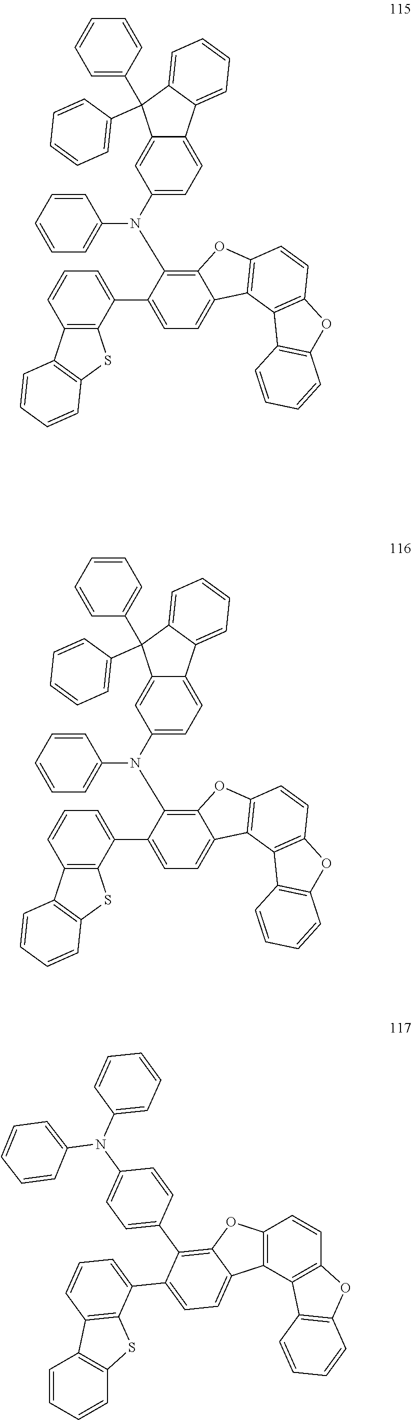 [SMILES: c1ccc(N(c2ccc3c(c2)C(c2ccccc2)(c2ccccc2)c2ccccc2-3)c2c(-c3cccc4c3sc3ccccc34)ccc3c2oc2ccc4oc5ccccc5c4c23)cc1 ; c1ccc(N(c2ccc3c(c2)C(c2ccccc2)(c2ccccc2)c2ccccc2-3)c2c(-c3cccc4c3sc3ccccc34)ccc3c2oc2ccc4oc5ccccc5c4c23)cc1 ; c1ccc(N(c2ccccc2)c2ccc(-c3c(-c4cccc5c4sc4ccccc45)ccc4c3oc3ccc5oc6ccccc6c5c34)cc2)cc1]

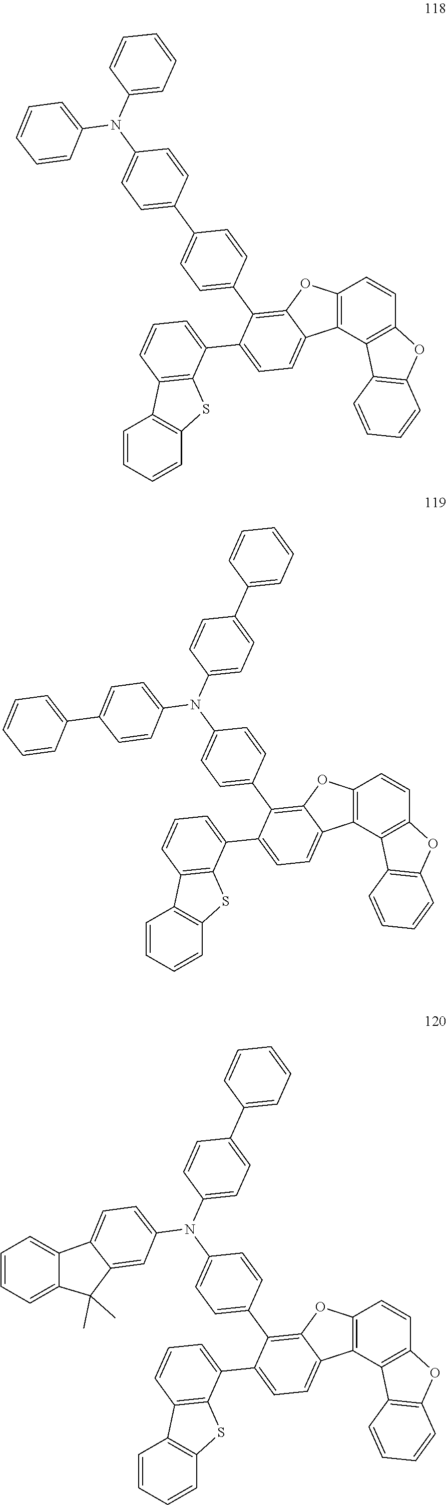 [SMILES: c1ccc(N(c2ccccc2)c2ccc(-c3ccc(-c4c(-c5cccc6c5sc5ccccc56)ccc5c4oc4ccc6oc7ccccc7c6c45)cc3)cc2)cc1 ; c1ccc(-c2ccc(N(c3ccc(-c4ccccc4)cc3)c3ccc(-c4c(-c5cccc6c5sc5ccccc56)ccc5c4oc4ccc6oc7ccccc7c6c45)cc3)cc2)cc1 ; CC1(C)c2ccccc2-c2ccc(N(c3ccc(-c4ccccc4)cc3)c3ccc(-c4c(-c5cccc6c5sc5ccccc56)ccc5c4oc4ccc6oc7ccccc7c6c45)cc3)cc21]

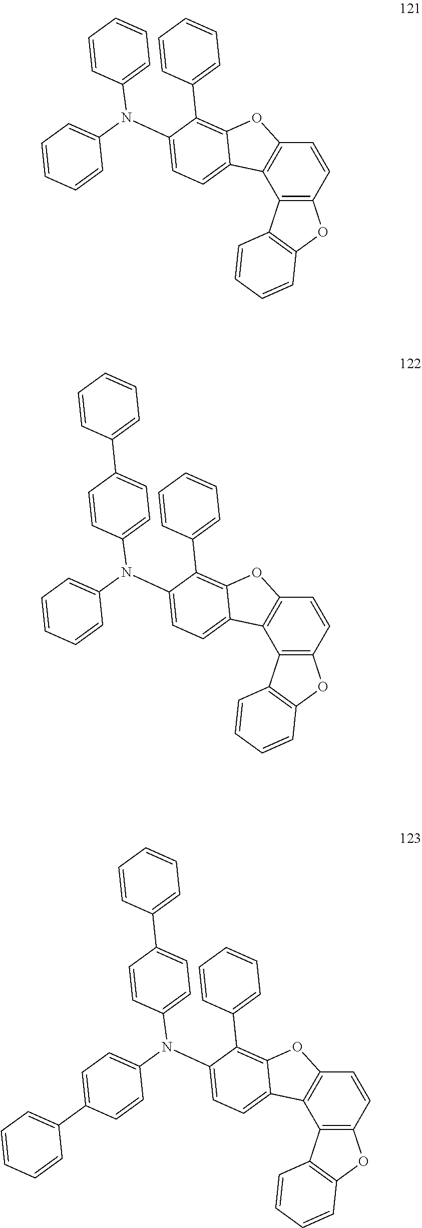 [SMILES: c1ccc(-c2c(N(c3ccccc3)c3ccccc3)ccc3c2oc2ccc4oc5ccccc5c4c23)cc1 ; c1ccc(-c2ccc(N(c3ccccc3)c3ccc4c(oc5ccc6oc7ccccc7c6c54)c3-c3ccccc3)cc2)cc1 ; c1ccc(-c2ccc(N(c3ccc(-c4ccccc4)cc3)c3ccc4c(oc5ccc6oc7ccccc7c6c54)c3-c3ccccc3)cc2)cc1]

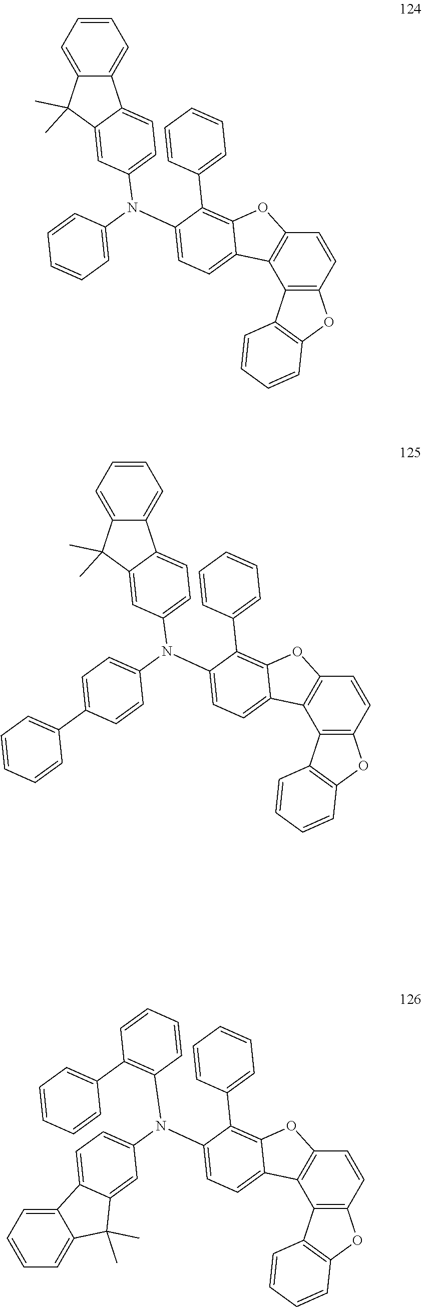 [SMILES: CC1(C)c2ccccc2-c2ccc(N(c3ccccc3)c3ccc4c(oc5ccc6oc7ccccc7c6c54)c3-c3ccccc3)cc21 ; CC1(C)c2ccccc2-c2ccc(N(c3ccc(-c4ccccc4)cc3)c3ccc4c(oc5ccc6oc7ccccc7c6c54)c3-c3ccccc3)cc21 ; CC1(C)c2ccccc2-c2ccc(N(c3ccccc3-c3ccccc3)c3ccc4c(oc5ccc6oc7ccccc7c6c54)c3-c3ccccc3)cc21]

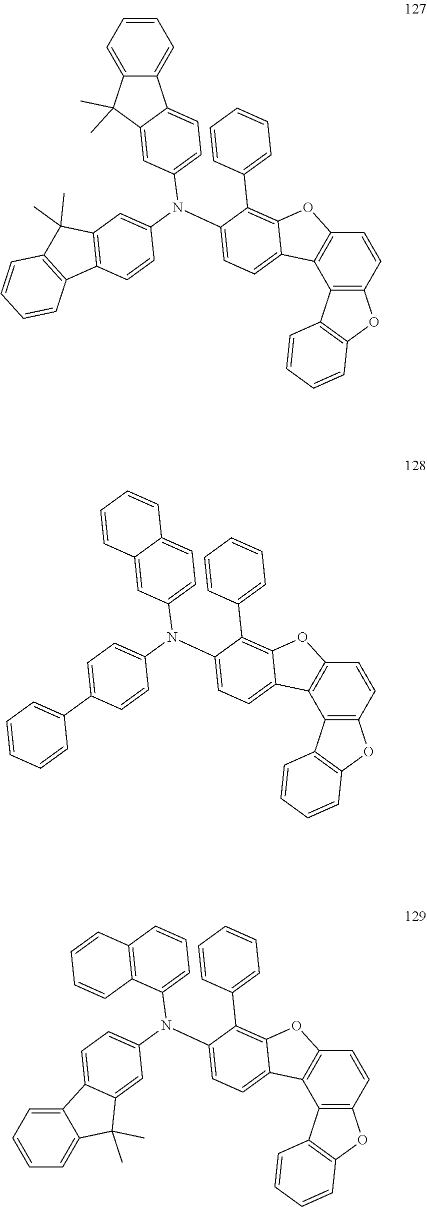 [SMILES: CC1(C)c2ccccc2-c2ccc(N(c3ccc4c(c3)C(C)(C)c3ccccc3-4)c3ccc4c(oc5ccc6oc7ccccc7c6c54)c3-c3ccccc3)cc21 ; c1ccc(-c2ccc(N(c3ccc4ccccc4c3)c3ccc4c(oc5ccc6oc7ccccc7c6c54)c3-c3ccccc3)cc2)cc1 ; CC1(C)c2ccccc2-c2ccc(N(c3ccc4c(oc5ccc6oc7ccccc7c6c54)c3-c3ccccc3)c3cccc4ccccc34)cc21]

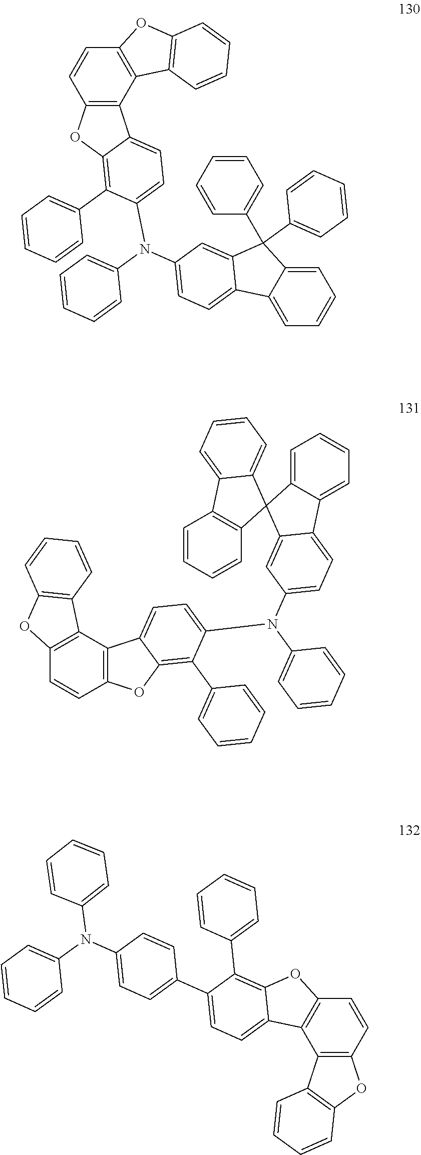 [SMILES: c1ccc(-c2c(N(c3ccccc3)c3ccc4c(c3)C(c3ccccc3)(c3ccccc3)c3ccccc3-4)ccc3c2oc2ccc4oc5ccccc5c4c23)cc1 ; c1ccc(-c2c(N(c3ccccc3)c3ccc4c(c3)C3(c5ccccc5-c5ccccc53)c3ccccc3-4)ccc3c2oc2ccc4oc5ccccc5c4c23)cc1 ; c1ccc(-c2c(-c3ccc(N(c4ccccc4)c4ccccc4)cc3)ccc3c2oc2ccc4oc5ccccc5c4c23)cc1]

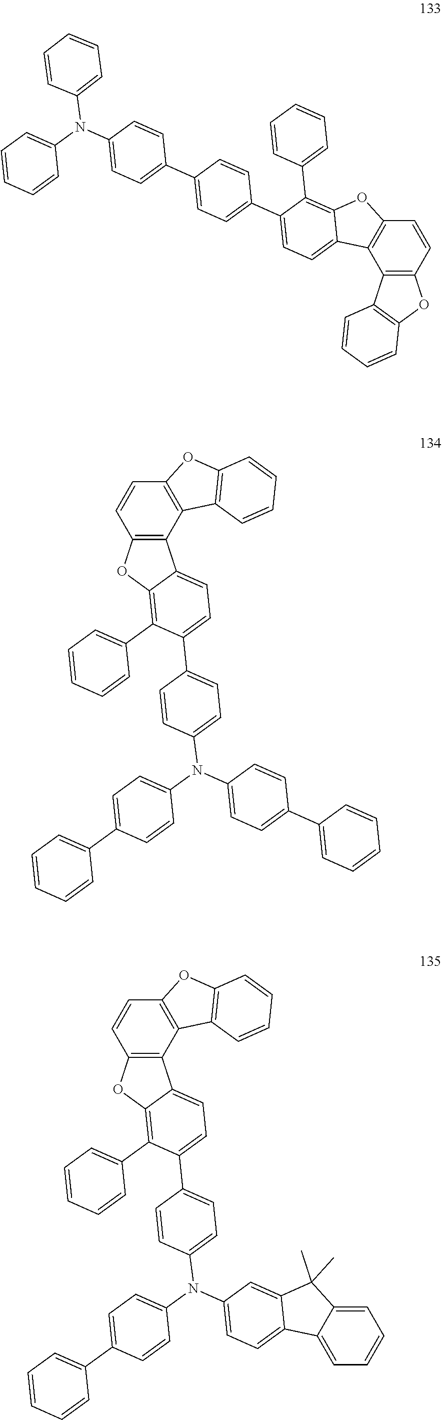 [SMILES: c1ccc(-c2c(-c3ccc(-c4ccc(N(c5ccccc5)c5ccccc5)cc4)cc3)ccc3c2oc2ccc4oc5ccccc5c4c23)cc1 ; c1ccc(-c2ccc(N(c3ccc(-c4ccccc4)cc3)c3ccc(-c4ccc5c(oc6ccc7oc8ccccc8c7c65)c4-c4ccccc4)cc3)cc2)cc1 ; CC1(C)c2ccccc2-c2ccc(N(c3ccc(-c4ccccc4)cc3)c3ccc(-c4ccc5c(oc6ccc7oc8ccccc8c7c65)c4-c4ccccc4)cc3)cc21]

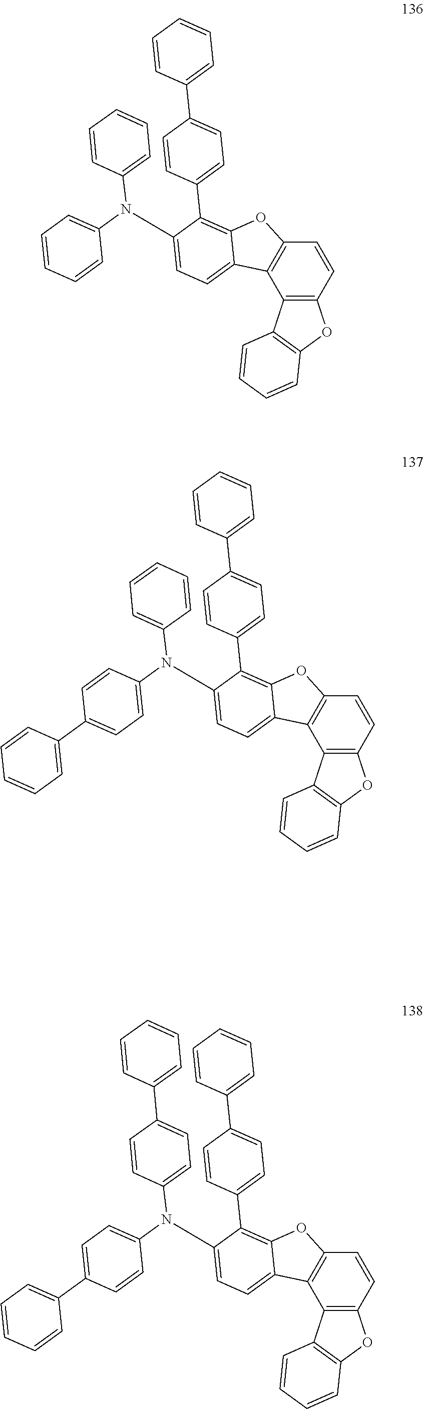 [SMILES: c1ccc(-c2ccc(-c3c(N(c4ccccc4)c4ccccc4)ccc4c3oc3ccc5oc6ccccc6c5c34)cc2)cc1 ; c1ccc(-c2ccc(-c3c(N(c4ccccc4)c4ccc(-c5ccccc5)cc4)ccc4c3oc3ccc5oc6ccccc6c5c34)cc2)cc1 ; c1ccc(-c2ccc(-c3c(N(c4ccc(-c5ccccc5)cc4)c4ccc(-c5ccccc5)cc4)ccc4c3oc3ccc5oc6ccccc6c5c34)cc2)cc1]

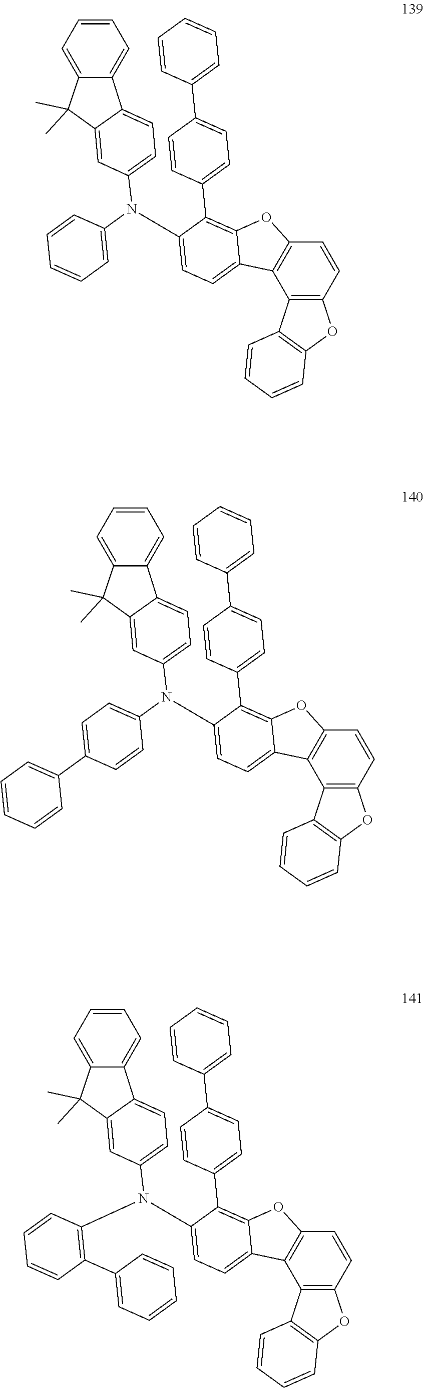 [SMILES: CC1(C)c2ccccc2-c2ccc(N(c3ccccc3)c3ccc4c(oc5ccc6oc7ccccc7c6c54)c3-c3ccc(-c4ccccc4)cc3)cc21 ; CC1(C)c2ccccc2-c2ccc(N(c3ccc(-c4ccccc4)cc3)c3ccc4c(oc5ccc6oc7ccccc7c6c54)c3-c3ccc(-c4ccccc4)cc3)cc21 ; CC1(C)c2ccccc2-c2ccc(N(c3ccccc3-c3ccccc3)c3ccc4c(oc5ccc6oc7ccccc7c6c54)c3-c3ccc(-c4ccccc4)cc3)cc21]

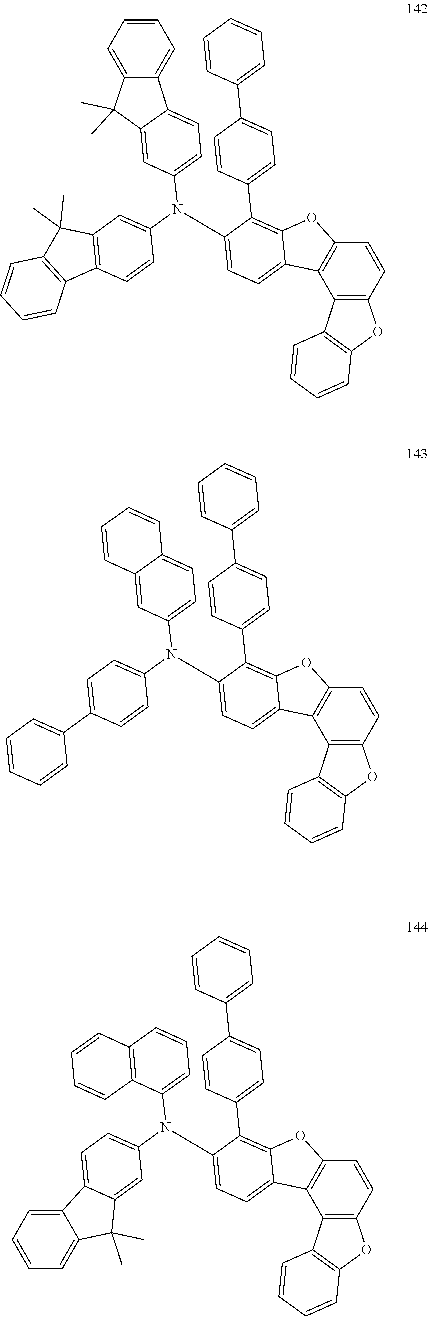 [SMILES: CC1(C)c2ccccc2-c2ccc(N(c3ccc4c(c3)C(C)(C)c3ccccc3-4)c3ccc4c(oc5ccc6oc7ccccc7c6c54)c3-c3ccc(-c4ccccc4)cc3)cc21 ; c1ccc(-c2ccc(-c3c(N(c4ccc(-c5ccccc5)cc4)c4ccc5ccccc5c4)ccc4c3oc3ccc5oc6ccccc6c5c34)cc2)cc1 ; CC1(C)c2ccccc2-c2ccc(N(c3ccc4c(oc5ccc6oc7ccccc7c6c54)c3-c3ccc(-c4ccccc4)cc3)c3cccc4ccccc34)cc21]

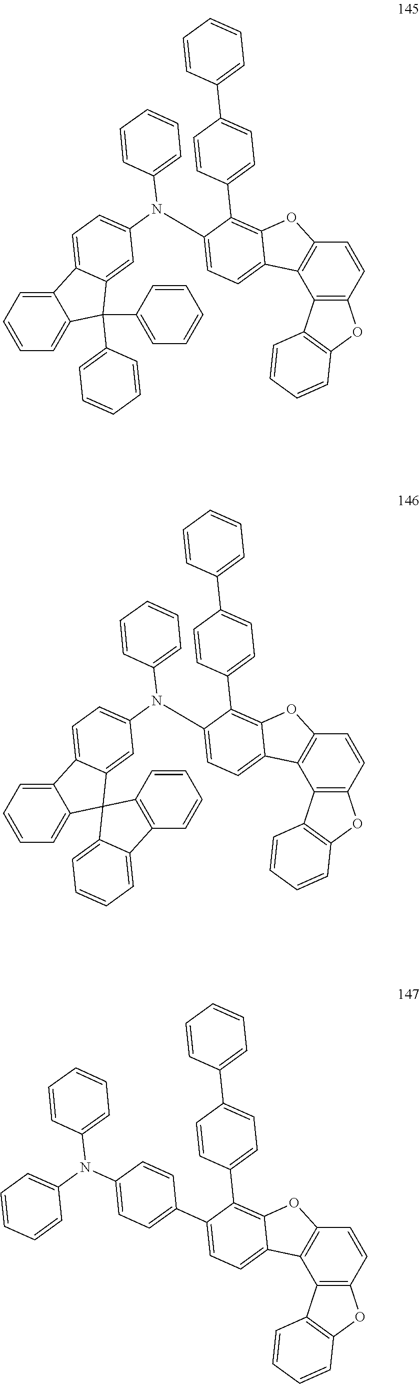 [SMILES: c1ccc(-c2ccc(-c3c(N(c4ccccc4)c4ccc5c(c4)C(c4ccccc4)(c4ccccc4)c4ccccc4-5)ccc4c3oc3ccc5oc6ccccc6c5c34)cc2)cc1 ; c1ccc(-c2ccc(-c3c(N(c4ccccc4)c4ccc5c(c4)C4(c6ccccc6-c6ccccc64)c4ccccc4-5)ccc4c3oc3ccc5oc6ccccc6c5c34)cc2)cc1 ; c1ccc(-c2ccc(-c3c(-c4ccc(N(c5ccccc5)c5ccccc5)cc4)ccc4c3oc3ccc5oc6ccccc6c5c34)cc2)cc1]

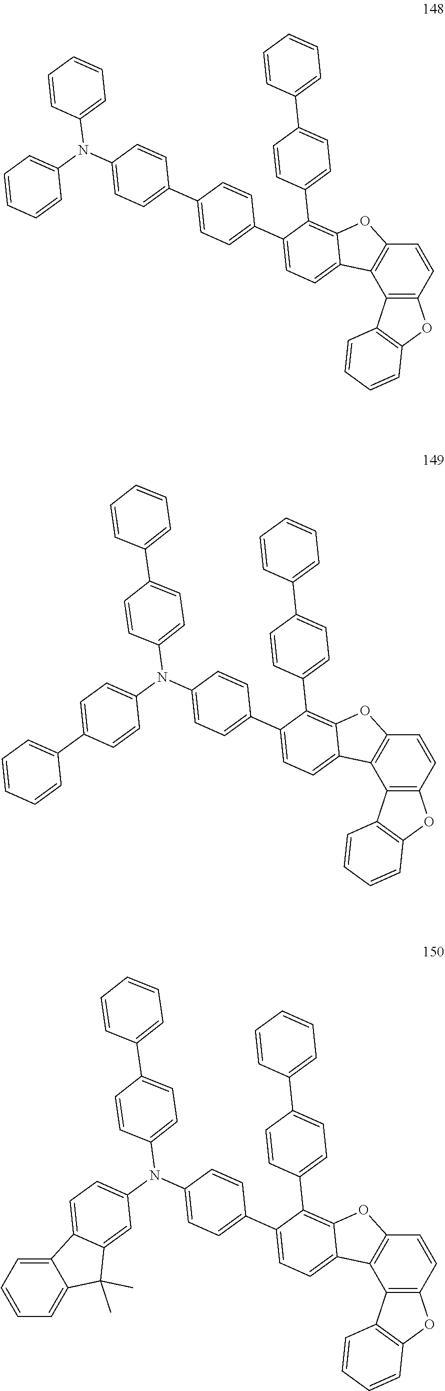 [SMILES: c1ccc(-c2ccc(-c3c(-c4ccc(-c5ccc(N(c6ccccc6)c6ccccc6)cc5)cc4)ccc4c3oc3ccc5oc6ccccc6c5c34)cc2)cc1 ; c1ccc(-c2ccc(-c3c(-c4ccc(N(c5ccc(-c6ccccc6)cc5)c5ccc(-c6ccccc6)cc5)cc4)ccc4c3oc3ccc5oc6ccccc6c5c34)cc2)cc1 ; CC1(C)c2ccccc2-c2ccc(N(c3ccc(-c4ccccc4)cc3)c3ccc(-c4ccc5c(oc6ccc7oc8ccccc8c7c65)c4-c4ccc(-c5ccccc5)cc4)cc3)cc21]

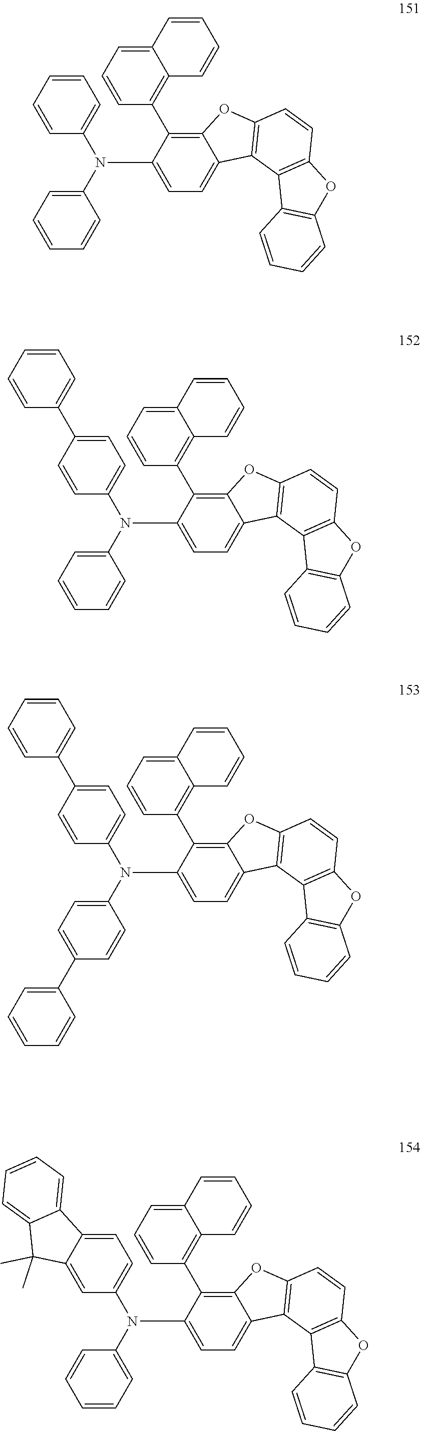 [SMILES: c1ccc(N(c2ccccc2)c2ccc3c(oc4ccc5oc6ccccc6c5c43)c2-c2cccc3ccccc23)cc1 ; c1ccc(-c2ccc(N(c3ccccc3)c3ccc4c(oc5ccc6oc7ccccc7c6c54)c3-c3cccc4ccccc34)cc2)cc1 ; c1ccc(-c2ccc(N(c3ccc(-c4ccccc4)cc3)c3ccc4c(oc5ccc6oc7ccccc7c6c54)c3-c3cccc4ccccc34)cc2)cc1 ; CC1(C)c2ccccc2-c2ccc(N(c3ccccc3)c3ccc4c(oc5ccc6oc7ccccc7c6c54)c3-c3cccc4ccccc34)cc21]

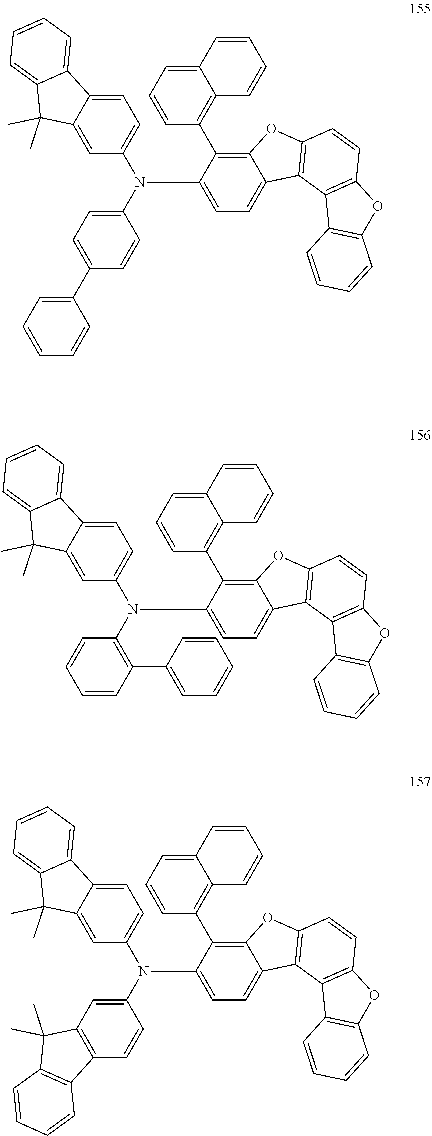 [SMILES: CC1(C)c2ccccc2-c2ccc(N(c3ccc(-c4ccccc4)cc3)c3ccc4c(oc5ccc6oc7ccccc7c6c54)c3-c3cccc4ccccc34)cc21 ; CC1(C)c2ccccc2-c2ccc(N(c3ccccc3-c3ccccc3)c3ccc4c(oc5ccc6oc7ccccc7c6c54)c3-c3cccc4ccccc34)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3cccc4ccccc34)cc21]

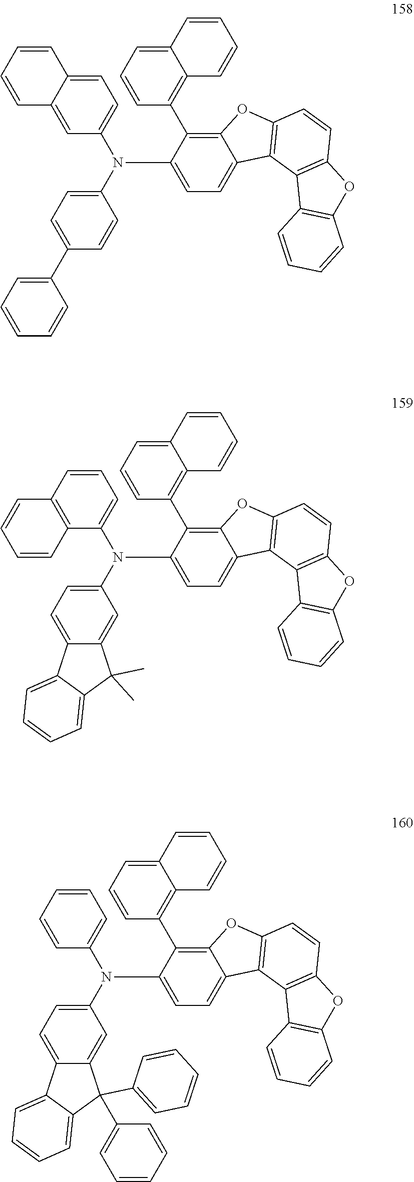 [SMILES: c1ccc(-c2ccc(N(c3ccc4ccccc4c3)c3ccc4c(oc5ccc6oc7ccccc7c6c54)c3-c3cccc4ccccc34)cc2)cc1 ; CC1(C)c2ccccc2-c2ccc(N(c3ccc4c(oc5ccc6oc7ccccc7c6c54)c3-c3cccc4ccccc34)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2cccc3ccccc23)cc1]

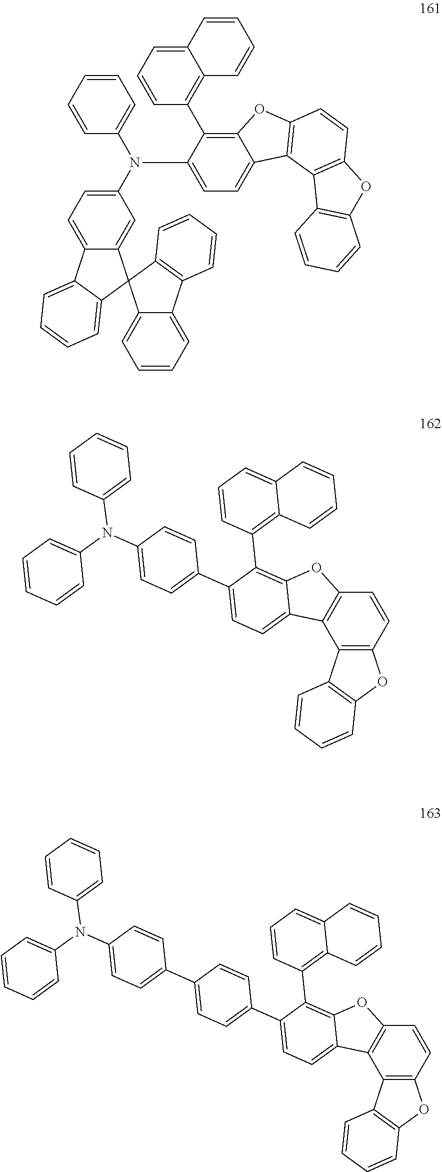 [SMILES: c1ccc(N(c2ccc3c(c2)C2(c4ccccc4-c4ccccc42)c2ccccc2-3)c2ccc3c(oc4ccc5oc6ccccc6c5c43)c2-c2cccc3ccccc23)cc1 ; c1ccc(N(c2ccccc2)c2ccc(-c3ccc4c(oc5ccc6oc7ccccc7c6c54)c3-c3cccc4ccccc34)cc2)cc1 ; c1ccc(N(c2ccccc2)c2ccc(-c3ccc(-c4ccc5c(oc6ccc7oc8ccccc8c7c65)c4-c4cccc5ccccc45)cc3)cc2)cc1]

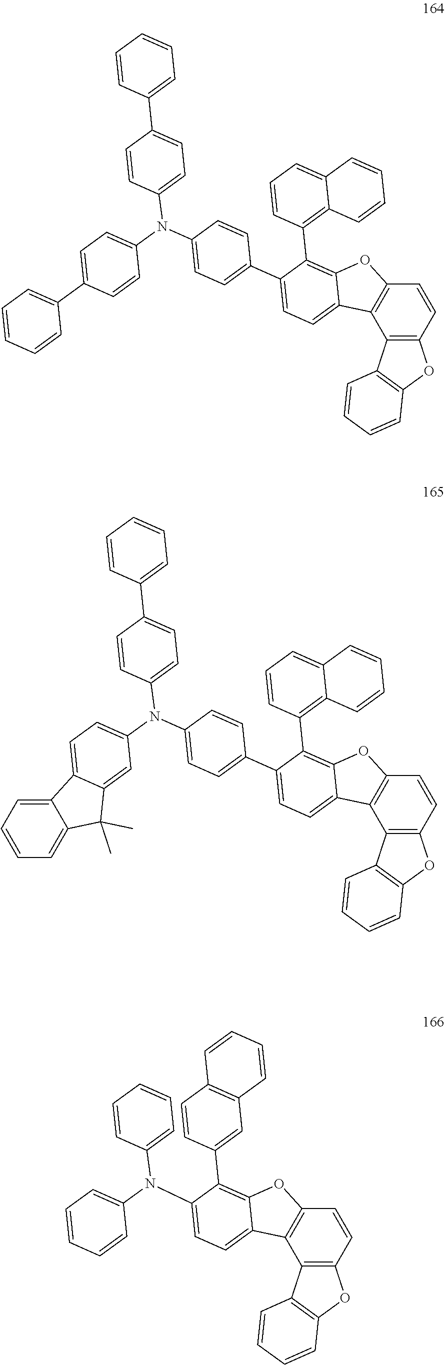 [SMILES: c1ccc(-c2ccc(N(c3ccc(-c4ccccc4)cc3)c3ccc(-c4ccc5c(oc6ccc7oc8ccccc8c7c65)c4-c4cccc5ccccc45)cc3)cc2)cc1 ; CC1(C)c2ccccc2-c2ccc(N(c3ccc(-c4ccccc4)cc3)c3ccc(-c4ccc5c(oc6ccc7oc8ccccc8c7c65)c4-c4cccc5ccccc45)cc3)cc21 ; c1ccc(N(c2ccccc2)c2ccc3c(oc4ccc5oc6ccccc6c5c43)c2-c2ccc3ccccc3c2)cc1]

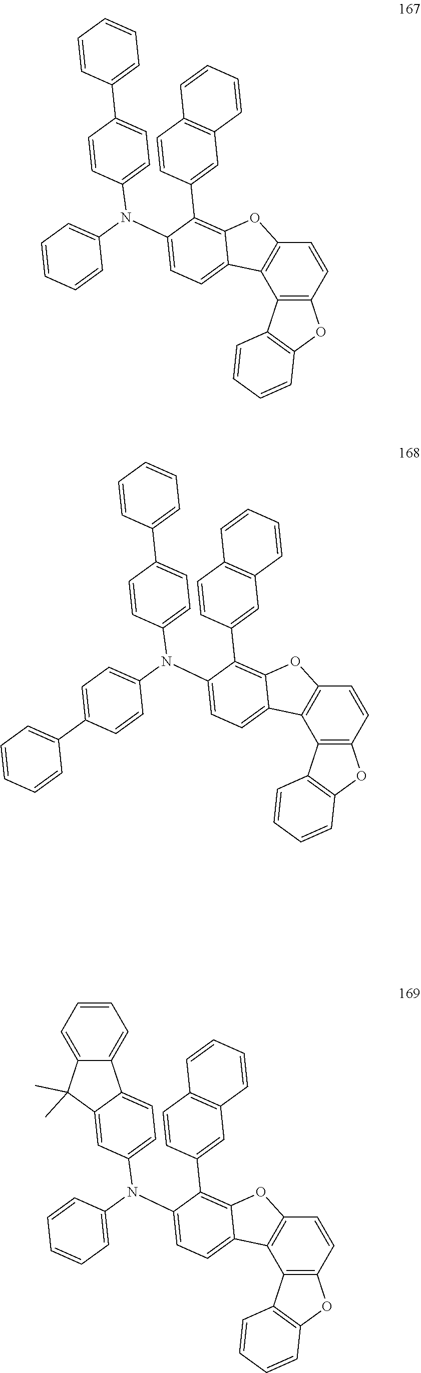 [SMILES: c1ccc(-c2ccc(N(c3ccccc3)c3ccc4c(oc5ccc6oc7ccccc7c6c54)c3-c3ccc4ccccc4c3)cc2)cc1 ; c1ccc(-c2ccc(N(c3ccc(-c4ccccc4)cc3)c3ccc4c(oc5ccc6oc7ccccc7c6c54)c3-c3ccc4ccccc4c3)cc2)cc1 ; CC1(C)c2ccccc2-c2ccc(N(c3ccccc3)c3ccc4c(oc5ccc6oc7ccccc7c6c54)c3-c3ccc4ccccc4c3)cc21]

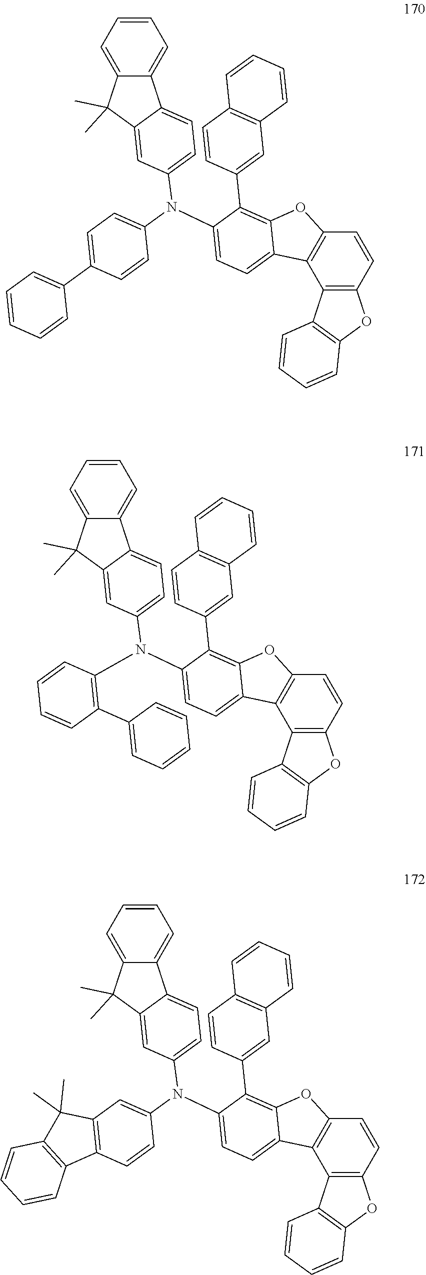 [SMILES: CC1(C)c2ccccc2-c2ccc(N(c3ccc(-c4ccccc4)cc3)c3ccc4c(oc5ccc6oc7ccccc7c6c54)c3-c3ccc4ccccc4c3)cc21 ; CC1(C)c2ccccc2-c2ccc(N(c3ccccc3-c3ccccc3)c3ccc4c(oc5ccc6oc7ccccc7c6c54)c3-c3ccc4ccccc4c3)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3ccc4ccccc4c3)cc21]

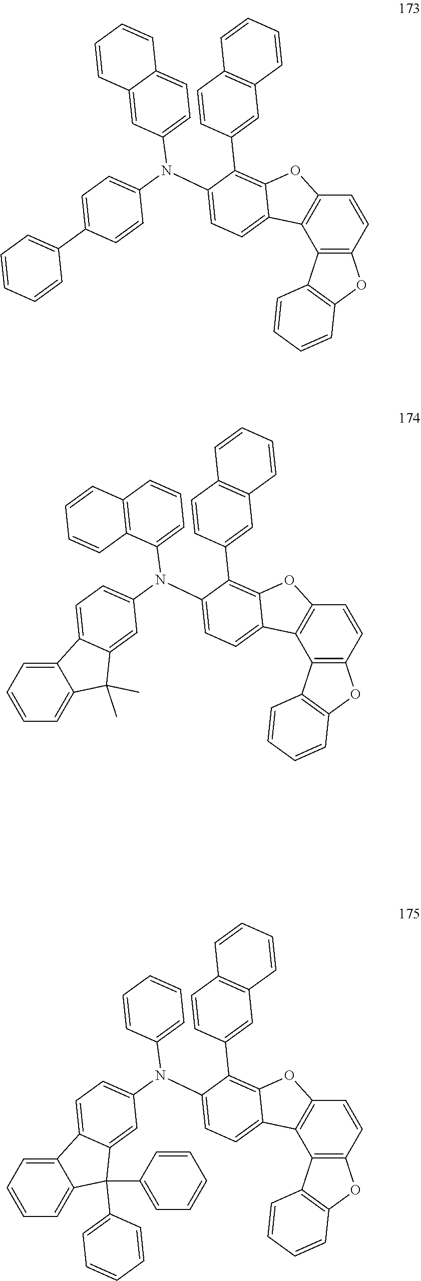 [SMILES: c1ccc(-c2ccc(N(c3ccc4ccccc4c3)c3ccc4c(oc5ccc6oc7ccccc7c6c54)c3-c3ccc4ccccc4c3)cc2)cc1 ; CC1(C)c2ccccc2-c2ccc(N(c3ccc4c(oc5ccc6oc7ccccc7c6c54)c3-c3ccc4ccccc4c3)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2ccc3ccccc3c2)cc1]

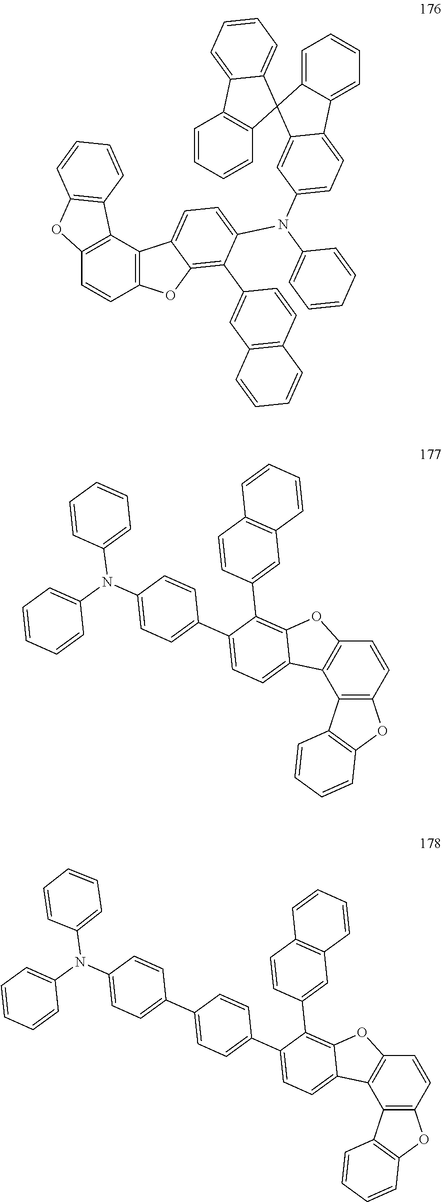 [SMILES: c1ccc(N(c2ccc3c(c2)C2(c4ccccc4-c4ccccc42)c2ccccc2-3)c2ccc3c(oc4ccc5oc6ccccc6c5c43)c2-c2ccc3ccccc3c2)cc1 ; c1ccc(N(c2ccccc2)c2ccc(-c3ccc4c(oc5ccc6oc7ccccc7c6c54)c3-c3ccc4ccccc4c3)cc2)cc1 ; c1ccc(N(c2ccccc2)c2ccc(-c3ccc(-c4ccc5c(oc6ccc7oc8ccccc8c7c65)c4-c4ccc5ccccc5c4)cc3)cc2)cc1]

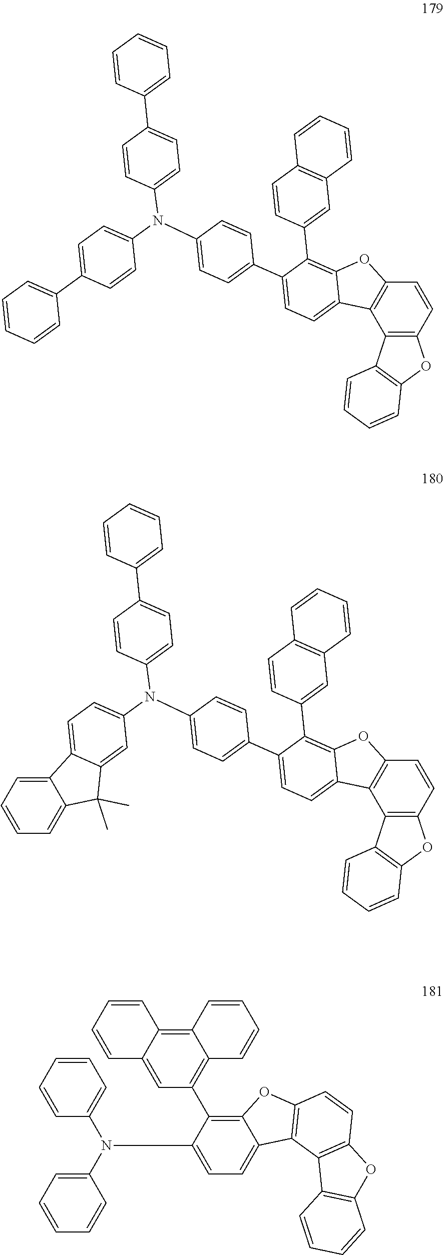 [SMILES: c1ccc(-c2ccc(N(c3ccc(-c4ccccc4)cc3)c3ccc(-c4ccc5c(oc6ccc7oc8ccccc8c7c65)c4-c4ccc5ccccc5c4)cc3)cc2)cc1 ; CC1(C)c2ccccc2-c2ccc(N(c3ccc(-c4ccccc4)cc3)c3ccc(-c4ccc5c(oc6ccc7oc8ccccc8c7c65)c4-c4ccc5ccccc5c4)cc3)cc21 ; c1ccc(N(c2ccccc2)c2ccc3c(oc4ccc5oc6ccccc6c5c43)c2-c2cc3ccccc3c3ccccc23)cc1]

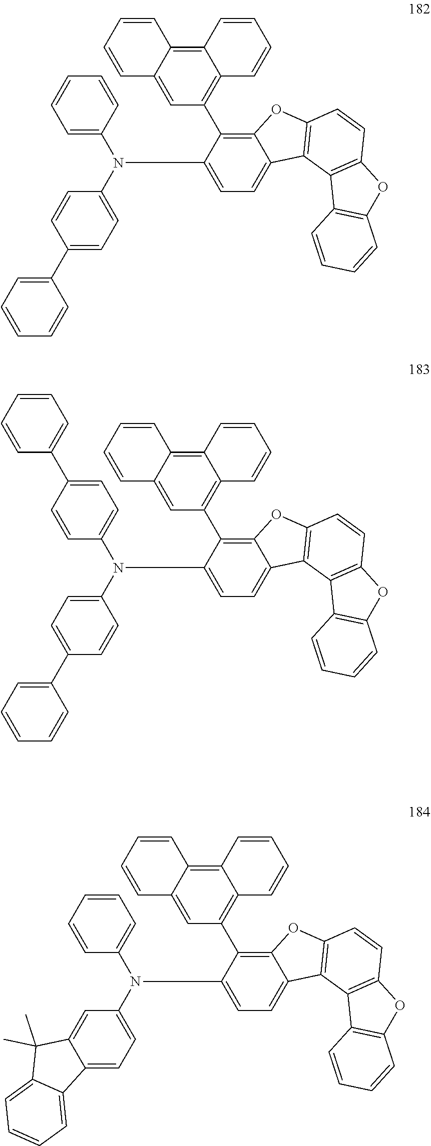 [SMILES: c1ccc(-c2ccc(N(c3ccccc3)c3ccc4c(oc5ccc6oc7ccccc7c6c54)c3-c3cc4ccccc4c4ccccc34)cc2)cc1 ; c1ccc(-c2ccc(N(c3ccc(-c4ccccc4)cc3)c3ccc4c(oc5ccc6oc7ccccc7c6c54)c3-c3cc4ccccc4c4ccccc34)cc2)cc1 ; CC1(C)c2ccccc2-c2ccc(N(c3ccccc3)c3ccc4c(oc5ccc6oc7ccccc7c6c54)c3-c3cc4ccccc4c4ccccc34)cc21]

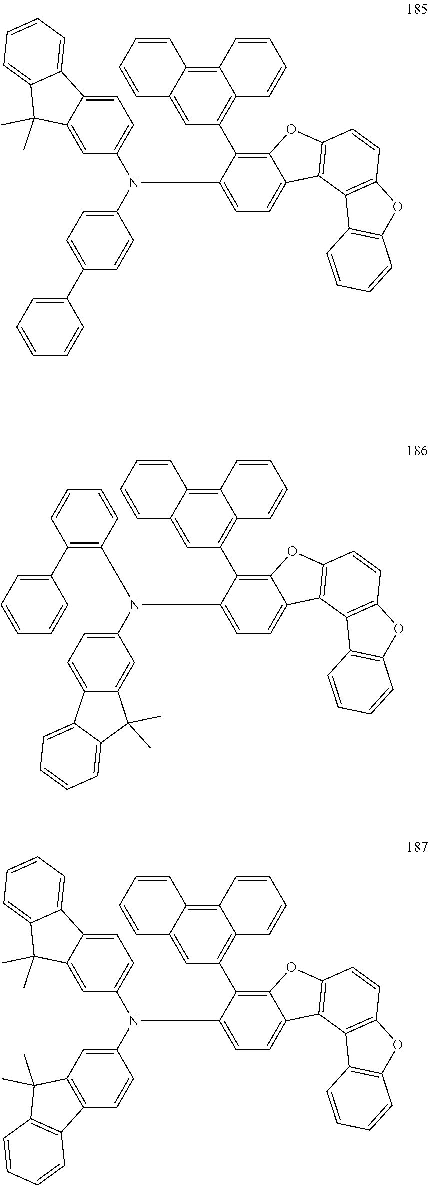 [SMILES: CC1(C)c2ccccc2-c2ccc(N(c3ccc(-c4ccccc4)cc3)c3ccc4c(oc5ccc6oc7ccccc7c6c54)c3-c3cc4ccccc4c4ccccc34)cc21 ; CC1(C)c2ccccc2-c2ccc(N(c3ccccc3-c3ccccc3)c3ccc4c(oc5ccc6oc7ccccc7c6c54)c3-c3cc4ccccc4c4ccccc34)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3cc4ccccc4c4ccccc34)cc21]

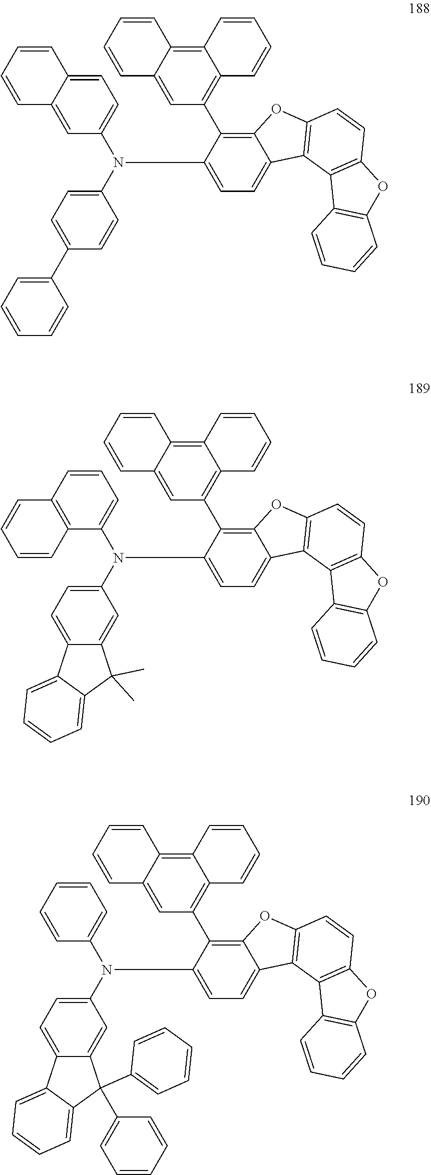 [SMILES: c1ccc(-c2ccc(N(c3ccc4ccccc4c3)c3ccc4c(oc5ccc6oc7ccccc7c6c54)c3-c3cc4ccccc4c4ccccc34)cc2)cc1 ; CC1(C)c2ccccc2-c2ccc(N(c3ccc4c(oc5ccc6oc7ccccc7c6c54)c3-c3cc4ccccc4c4ccccc34)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2cc3ccccc3c3ccccc23)cc1]

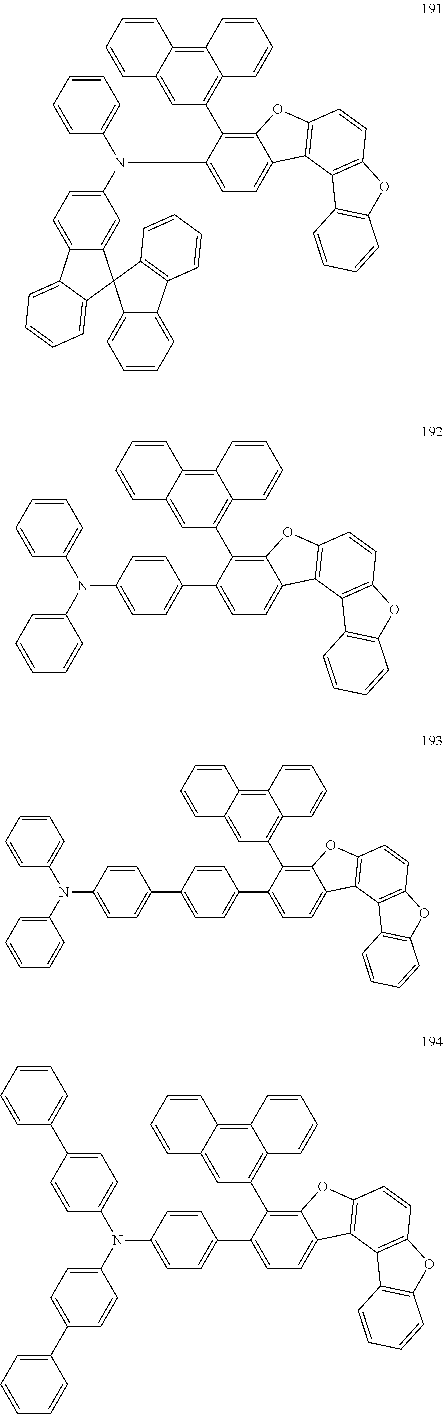 [SMILES: c1ccc(N(c2ccc3c(c2)C2(c4ccccc4-c4ccccc42)c2ccccc2-3)c2ccc3c(oc4ccc5oc6ccccc6c5c43)c2-c2cc3ccccc3c3ccccc23)cc1 ; c1ccc(N(c2ccccc2)c2ccc(-c3ccc4c(oc5ccc6oc7ccccc7c6c54)c3-c3cc4ccccc4c4ccccc34)cc2)cc1 ; c1ccc(N(c2ccccc2)c2ccc(-c3ccc(-c4ccc5c(oc6ccc7oc8ccccc8c7c65)c4-c4cc5ccccc5c5ccccc45)cc3)cc2)cc1 ; c1ccc(-c2ccc(N(c3ccc(-c4ccccc4)cc3)c3ccc(-c4ccc5c(oc6ccc7oc8ccccc8c7c65)c4-c4cc5ccccc5c5ccccc45)cc3)cc2)cc1]

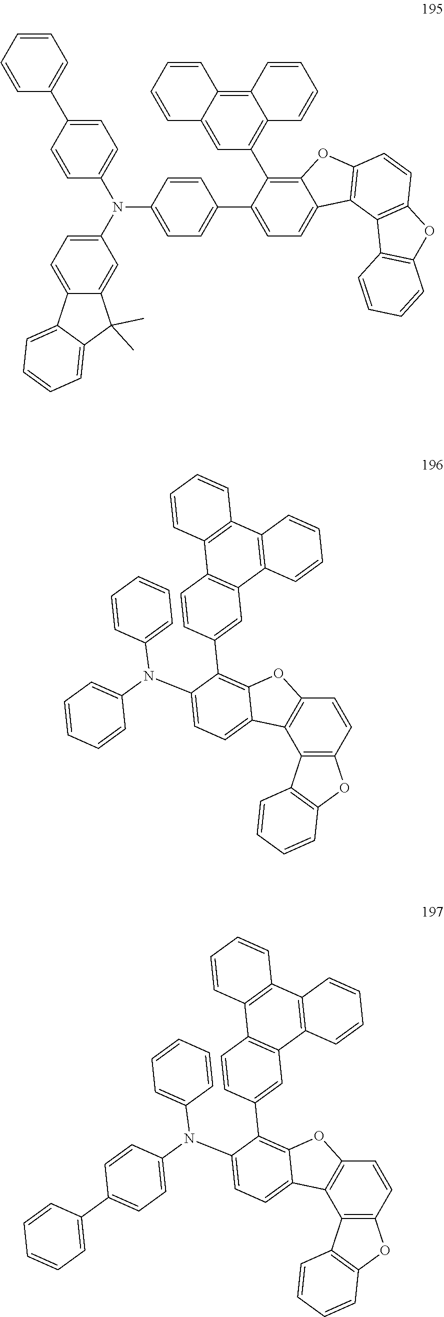 [SMILES: CC1(C)c2ccccc2-c2ccc(N(c3ccc(-c4ccccc4)cc3)c3ccc(-c4ccc5c(oc6ccc7oc8ccccc8c7c65)c4-c4cc5ccccc5c5ccccc45)cc3)cc21 ; c1ccc(N(c2ccccc2)c2ccc3c(oc4ccc5oc6ccccc6c5c43)c2-c2ccc3c4ccccc4c4ccccc4c3c2)cc1 ; c1ccc(-c2ccc(N(c3ccccc3)c3ccc4c(oc5ccc6oc7ccccc7c6c54)c3-c3ccc4c5ccccc5c5ccccc5c4c3)cc2)cc1]

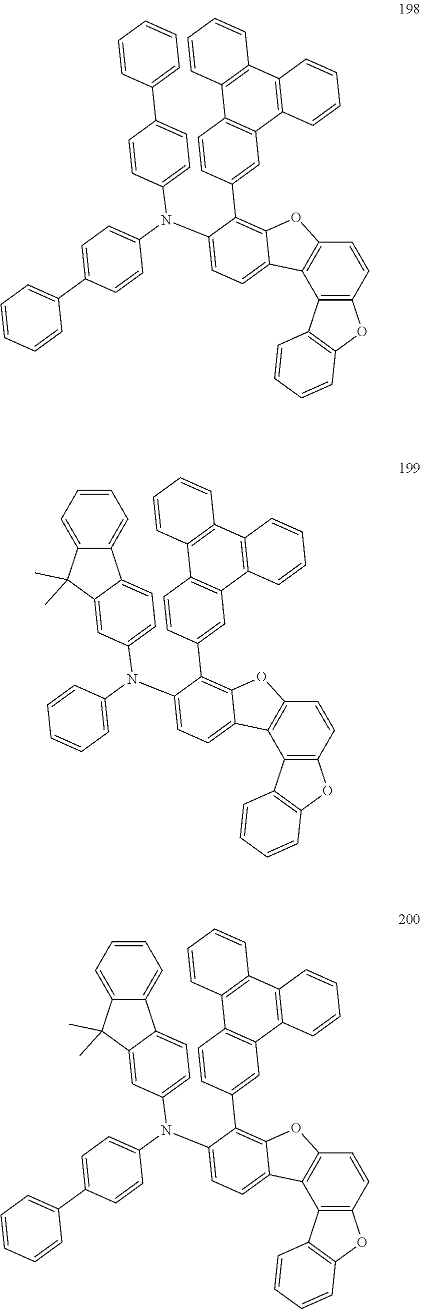 [SMILES: c1ccc(-c2ccc(N(c3ccc(-c4ccccc4)cc3)c3ccc4c(oc5ccc6oc7ccccc7c6c54)c3-c3ccc4c5ccccc5c5ccccc5c4c3)cc2)cc1 ; CC1(C)c2ccccc2-c2ccc(N(c3ccccc3)c3ccc4c(oc5ccc6oc7ccccc7c6c54)c3-c3ccc4c5ccccc5c5ccccc5c4c3)cc21 ; CC1(C)c2ccccc2-c2ccc(N(c3ccc(-c4ccccc4)cc3)c3ccc4c(oc5ccc6oc7ccccc7c6c54)c3-c3ccc4c5ccccc5c5ccccc5c4c3)cc21]

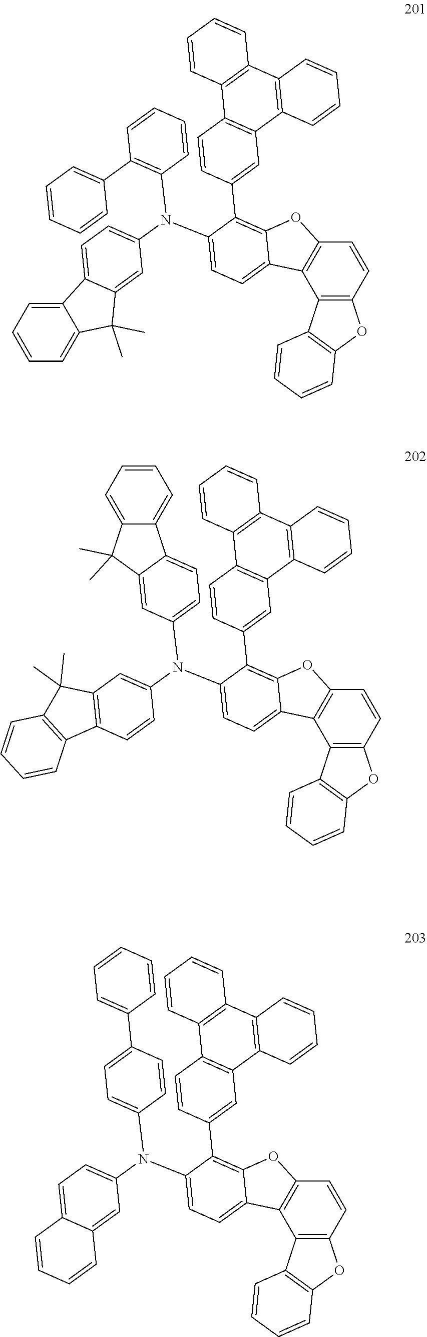 [SMILES: CC1(C)c2ccccc2-c2ccc(N(c3ccccc3-c3ccccc3)c3ccc4c(oc5ccc6oc7ccccc7c6c54)c3-c3ccc4c5ccccc5c5ccccc5c4c3)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3ccc4c5ccccc5c5ccccc5c4c3)cc21 ; c1ccc(-c2ccc(N(c3ccc4ccccc4c3)c3ccc4c(oc5ccc6oc7ccccc7c6c54)c3-c3ccc4c5ccccc5c5ccccc5c4c3)cc2)cc1]

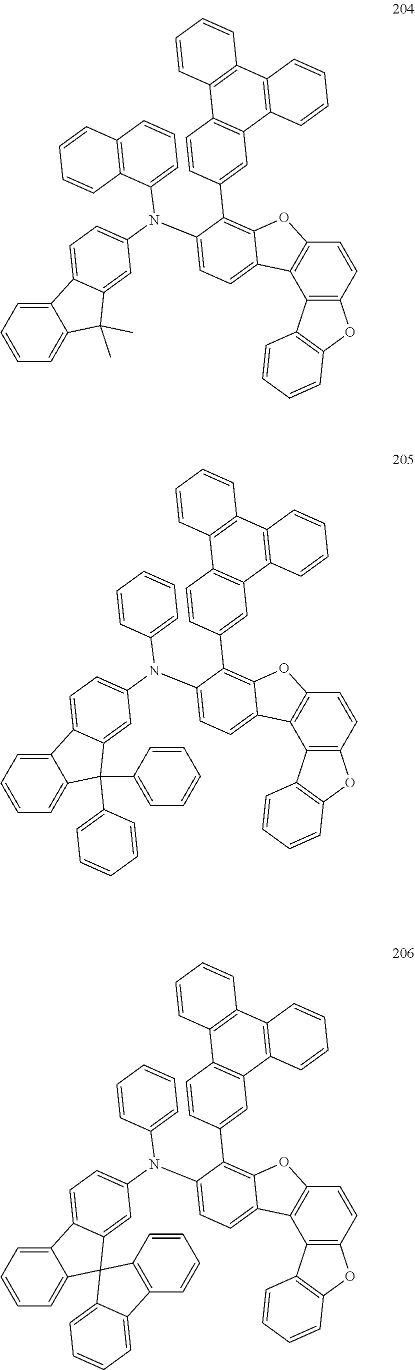 [SMILES: CC1(C)c2ccccc2-c2ccc(N(c3ccc4c(oc5ccc6oc7ccccc7c6c54)c3-c3ccc4c5ccccc5c5ccccc5c4c3)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2ccc3c4ccccc4c4ccccc4c3c2)cc1 ; c1ccc(N(c2ccc3c(c2)C2(c4ccccc4-c4ccccc42)c2ccccc2-3)c2ccc3c(oc4ccc5oc6ccccc6c5c43)c2-c2ccc3c4ccccc4c4ccccc4c3c2)cc1]

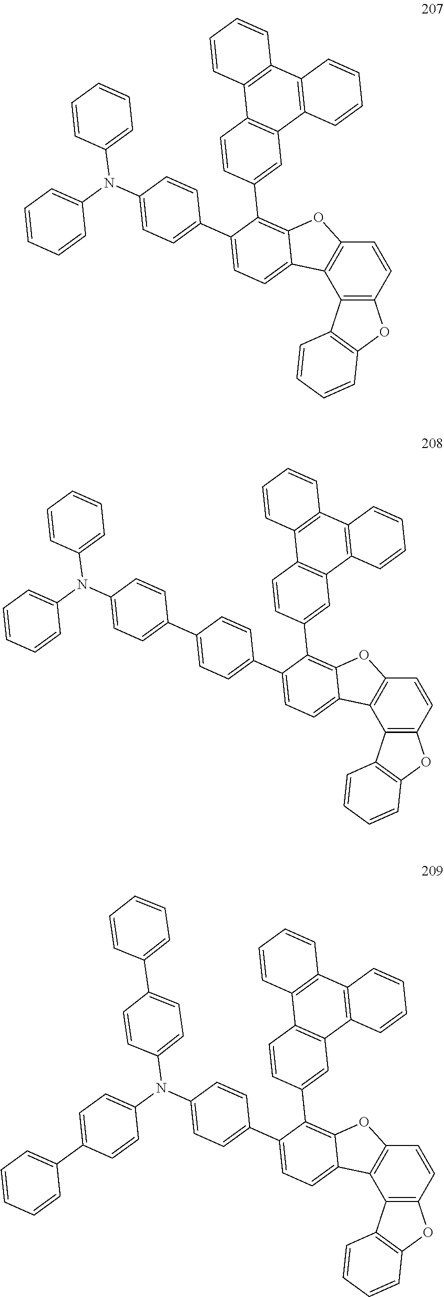 [SMILES: c1ccc(N(c2ccccc2)c2ccc(-c3ccc4c(oc5ccc6oc7ccccc7c6c54)c3-c3ccc4c5ccccc5c5ccccc5c4c3)cc2)cc1 ; c1ccc(N(c2ccccc2)c2ccc(-c3ccc(-c4ccc5c(oc6ccc7oc8ccccc8c7c65)c4-c4ccc5c6ccccc6c6ccccc6c5c4)cc3)cc2)cc1 ; c1ccc(-c2ccc(N(c3ccc(-c4ccccc4)cc3)c3ccc(-c4ccc5c(oc6ccc7oc8ccccc8c7c65)c4-c4ccc5c6ccccc6c6ccccc6c5c4)cc3)cc2)cc1]

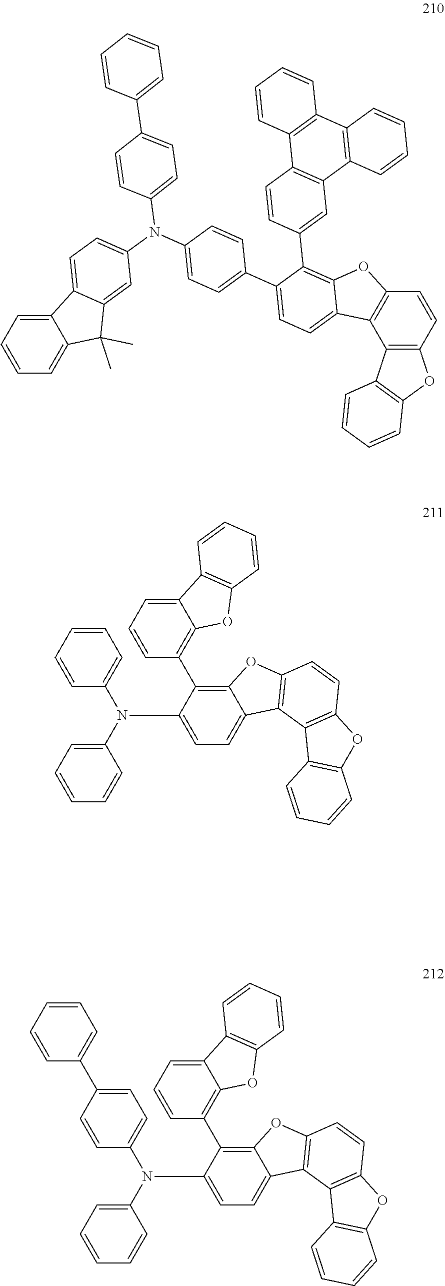 [SMILES: CC1(C)c2ccccc2-c2ccc(N(c3ccc(-c4ccccc4)cc3)c3ccc(-c4ccc5c(oc6ccc7oc8ccccc8c7c65)c4-c4ccc5c6ccccc6c6ccccc6c5c4)cc3)cc21 ; c1ccc(N(c2ccccc2)c2ccc3c(oc4ccc5oc6ccccc6c5c43)c2-c2cccc3c2oc2ccccc23)cc1 ; c1ccc(-c2ccc(N(c3ccccc3)c3ccc4c(oc5ccc6oc7ccccc7c6c54)c3-c3cccc4c3oc3ccccc34)cc2)cc1]

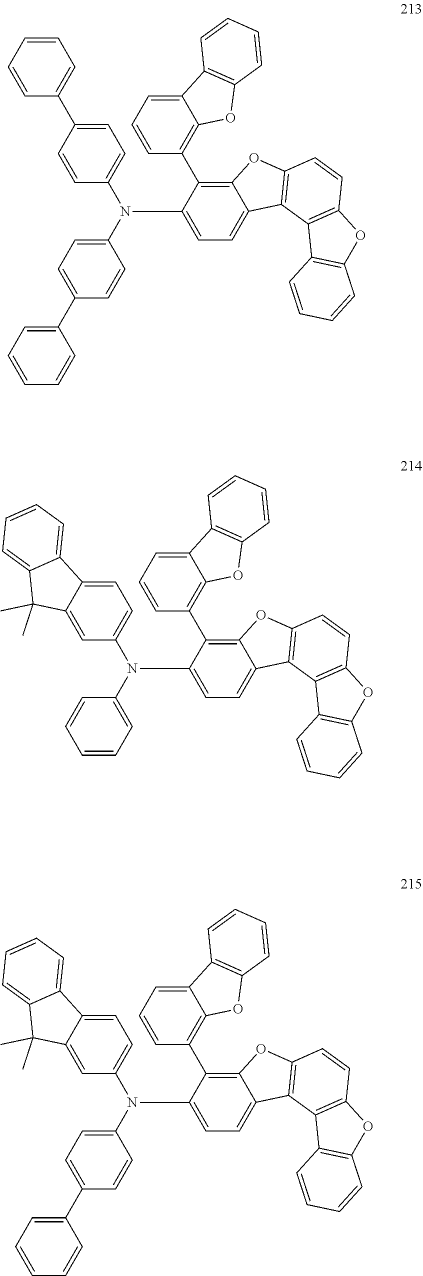 [SMILES: c1ccc(-c2ccc(N(c3ccc(-c4ccccc4)cc3)c3ccc4c(oc5ccc6oc7ccccc7c6c54)c3-c3cccc4c3oc3ccccc34)cc2)cc1 ; CC1(C)c2ccccc2-c2ccc(N(c3ccccc3)c3ccc4c(oc5ccc6oc7ccccc7c6c54)c3-c3cccc4c3oc3ccccc34)cc21 ; CC1(C)c2ccccc2-c2ccc(N(c3ccc(-c4ccccc4)cc3)c3ccc4c(oc5ccc6oc7ccccc7c6c54)c3-c3cccc4c3oc3ccccc34)cc21]

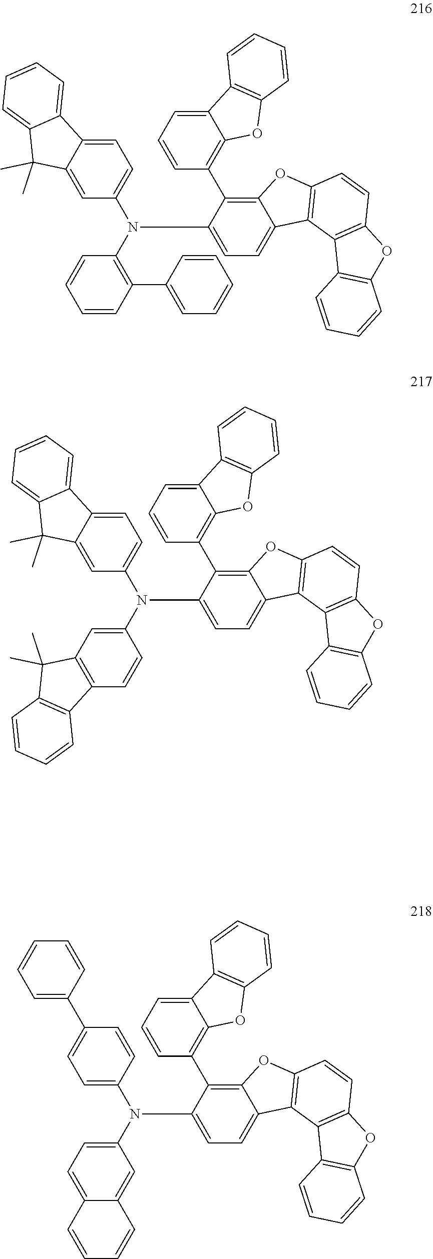 [SMILES: CC1(C)c2ccccc2-c2ccc(N(c3ccccc3-c3ccccc3)c3ccc4c(oc5ccc6oc7ccccc7c6c54)c3-c3cccc4c3oc3ccccc34)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3cccc4c3oc3ccccc34)cc21 ; c1ccc(-c2ccc(N(c3ccc4ccccc4c3)c3ccc4c(oc5ccc6oc7ccccc7c6c54)c3-c3cccc4c3oc3ccccc34)cc2)cc1]

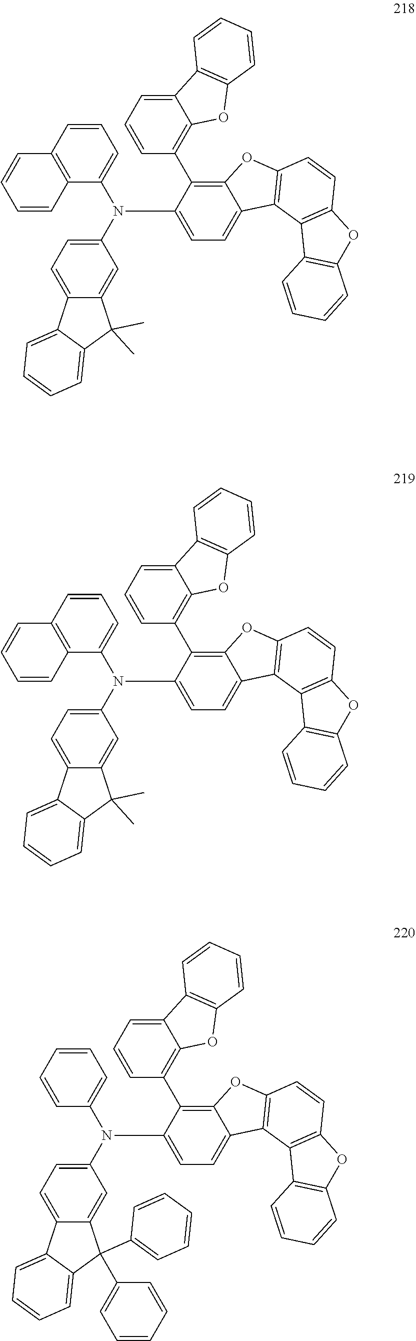 [SMILES: CC1(C)c2ccccc2-c2ccc(N(c3ccc4c(oc5ccc6oc7ccccc7c6c54)c3-c3cccc4c3oc3ccccc34)c3cccc4ccccc34)cc21 ; CC1(C)c2ccccc2-c2ccc(N(c3ccc4c(oc5ccc6oc7ccccc7c6c54)c3-c3cccc4c3oc3ccccc34)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2cccc3c2oc2ccccc23)cc1]

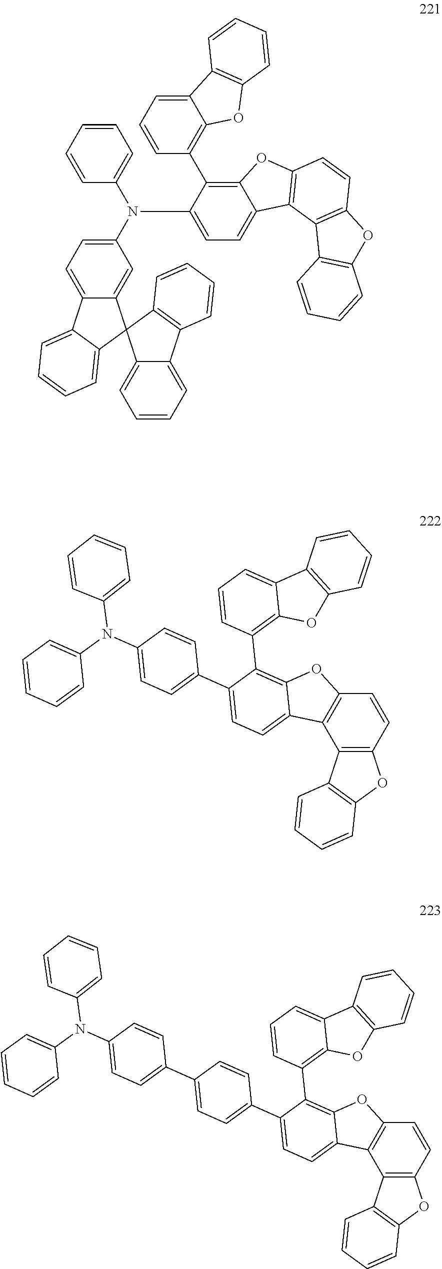 [SMILES: c1ccc(N(c2ccc3c(c2)C2(c4ccccc4-c4ccccc42)c2ccccc2-3)c2ccc3c(oc4ccc5oc6ccccc6c5c43)c2-c2cccc3c2oc2ccccc23)cc1 ; c1ccc(N(c2ccccc2)c2ccc(-c3ccc4c(oc5ccc6oc7ccccc7c6c54)c3-c3cccc4c3oc3ccccc34)cc2)cc1 ; c1ccc(N(c2ccccc2)c2ccc(-c3ccc(-c4ccc5c(oc6ccc7oc8ccccc8c7c65)c4-c4cccc5c4oc4ccccc45)cc3)cc2)cc1]

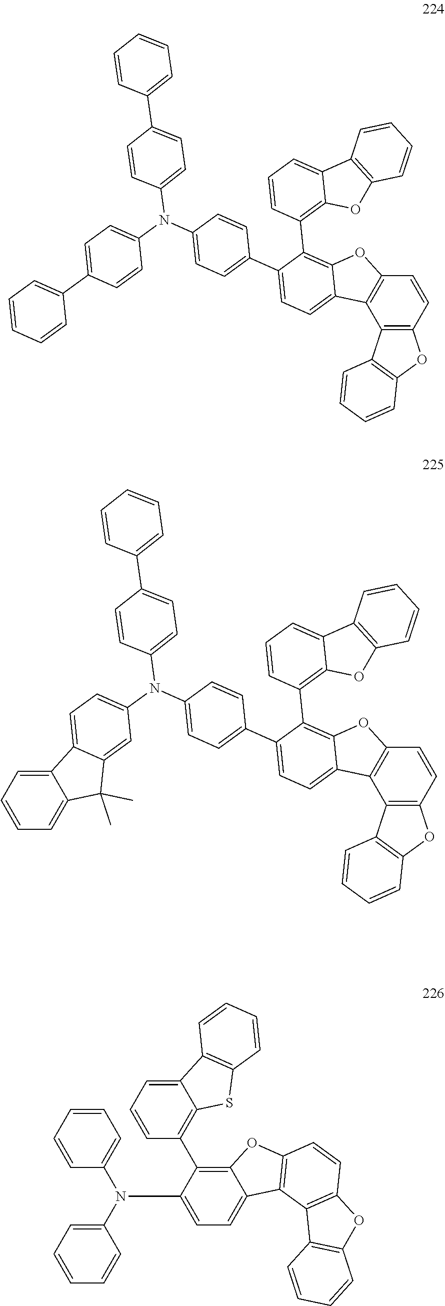 [SMILES: c1ccc(-c2ccc(N(c3ccc(-c4ccccc4)cc3)c3ccc(-c4ccc5c(oc6ccc7oc8ccccc8c7c65)c4-c4cccc5c4oc4ccccc45)cc3)cc2)cc1 ; CC1(C)c2ccccc2-c2ccc(N(c3ccc(-c4ccccc4)cc3)c3ccc(-c4ccc5c(oc6ccc7oc8ccccc8c7c65)c4-c4cccc5c4oc4ccccc45)cc3)cc21 ; c1ccc(N(c2ccccc2)c2ccc3c(oc4ccc5oc6ccccc6c5c43)c2-c2cccc3c2sc2ccccc23)cc1]

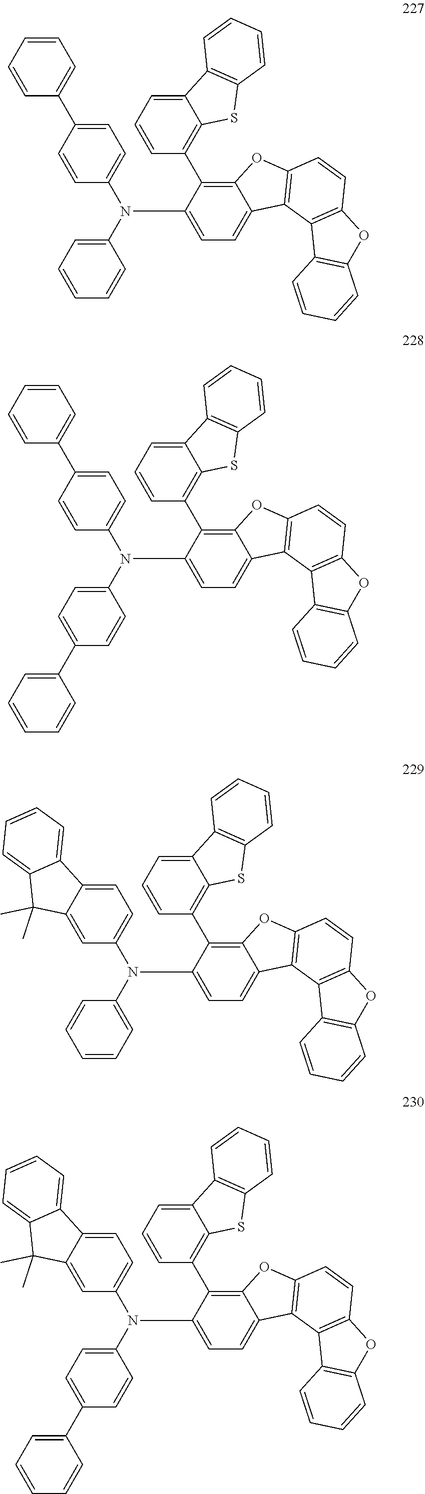 [SMILES: c1ccc(-c2ccc(N(c3ccccc3)c3ccc4c(oc5ccc6oc7ccccc7c6c54)c3-c3cccc4c3sc3ccccc34)cc2)cc1 ; c1ccc(-c2ccc(N(c3ccc(-c4ccccc4)cc3)c3ccc4c(oc5ccc6oc7ccccc7c6c54)c3-c3cccc4c3sc3ccccc34)cc2)cc1 ; CC1(C)c2ccccc2-c2ccc(N(c3ccccc3)c3ccc4c(oc5ccc6oc7ccccc7c6c54)c3-c3cccc4c3sc3ccccc34)cc21 ; CC1(C)c2ccccc2-c2ccc(N(c3ccc(-c4ccccc4)cc3)c3ccc4c(oc5ccc6oc7ccccc7c6c54)c3-c3cccc4c3sc3ccccc34)cc21]

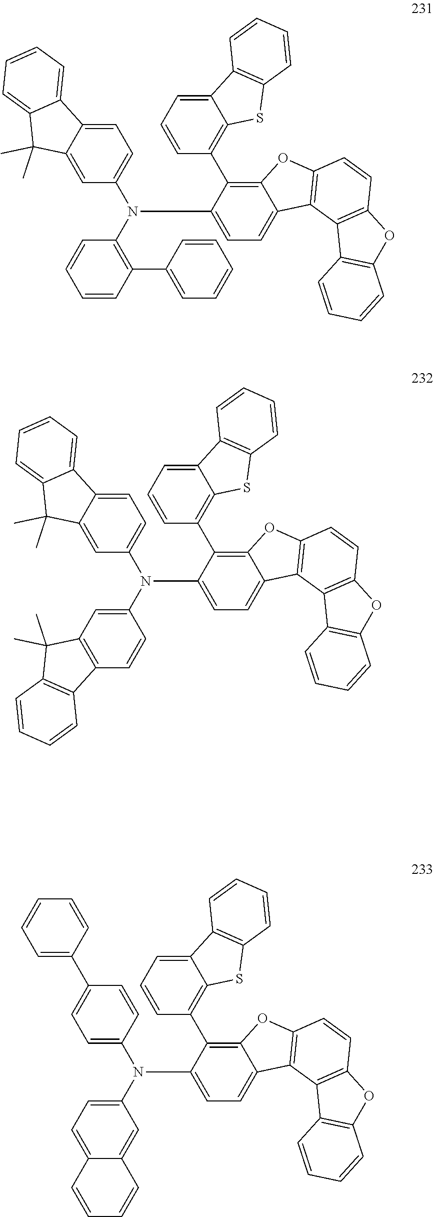 [SMILES: CC1(C)c2ccccc2-c2ccc(N(c3ccccc3-c3ccccc3)c3ccc4c(oc5ccc6oc7ccccc7c6c54)c3-c3cccc4c3sc3ccccc34)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3cccc4c3sc3ccccc34)cc21 ; c1ccc(-c2ccc(N(c3ccc4ccccc4c3)c3ccc4c(oc5ccc6oc7ccccc7c6c54)c3-c3cccc4c3sc3ccccc34)cc2)cc1]

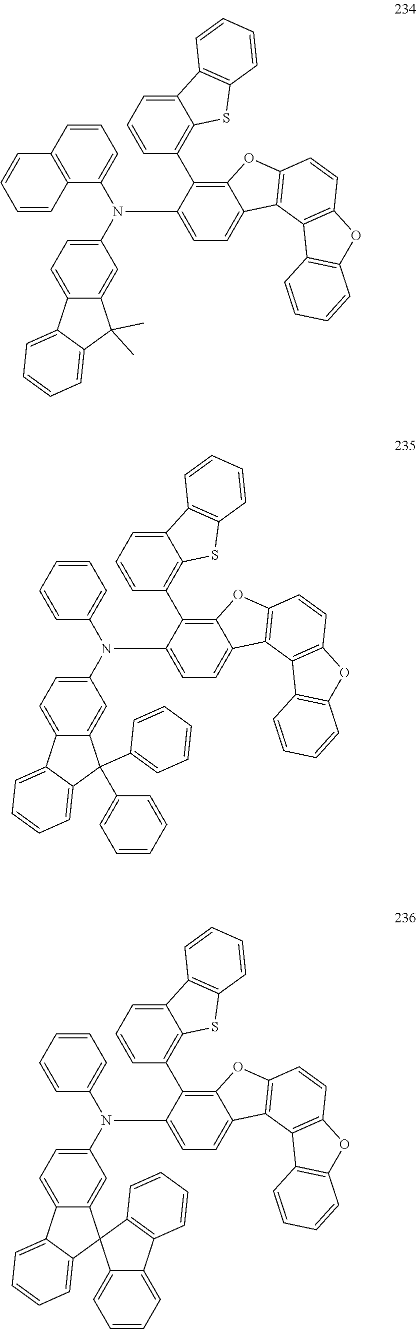 [SMILES: CC1(C)c2ccccc2-c2ccc(N(c3ccc4c(oc5ccc6oc7ccccc7c6c54)c3-c3cccc4c3sc3ccccc34)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2cccc3c2sc2ccccc23)cc1 ; c1ccc(N(c2ccc3c(c2)C2(c4ccccc4-c4ccccc42)c2ccccc2-3)c2ccc3c(oc4ccc5oc6ccccc6c5c43)c2-c2cccc3c2sc2ccccc23)cc1]

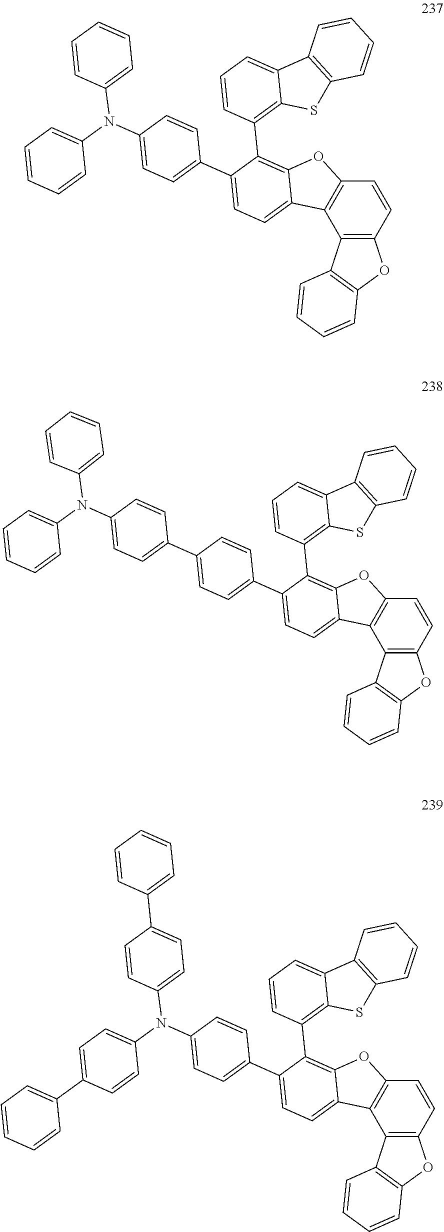 [SMILES: c1ccc(N(c2ccccc2)c2ccc(-c3ccc4c(oc5ccc6oc7ccccc7c6c54)c3-c3cccc4c3sc3ccccc34)cc2)cc1 ; c1ccc(N(c2ccccc2)c2ccc(-c3ccc(-c4ccc5c(oc6ccc7oc8ccccc8c7c65)c4-c4cccc5c4sc4ccccc45)cc3)cc2)cc1 ; c1ccc(-c2ccc(N(c3ccc(-c4ccccc4)cc3)c3ccc(-c4ccc5c(oc6ccc7oc8ccccc8c7c65)c4-c4cccc5c4sc4ccccc45)cc3)cc2)cc1]

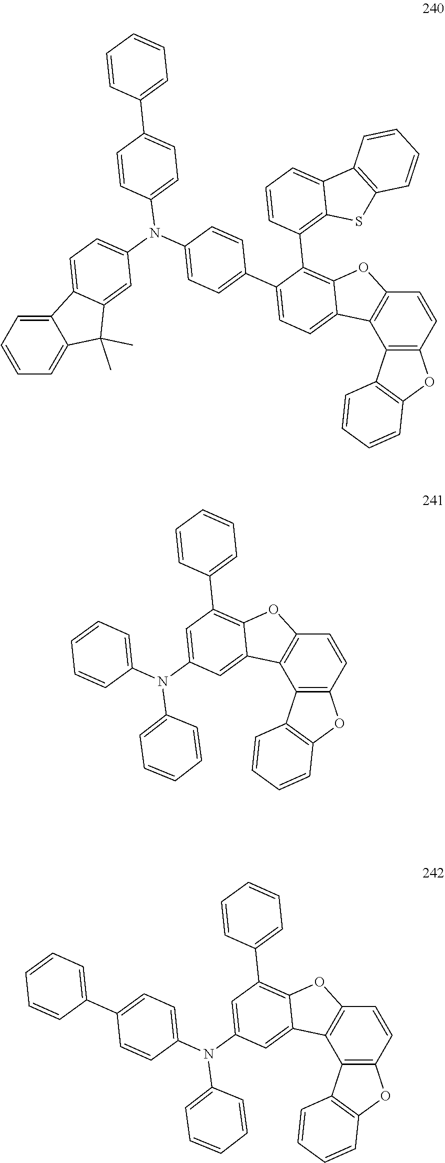 [SMILES: CC1(C)c2ccccc2-c2ccc(N(c3ccc(-c4ccccc4)cc3)c3ccc(-c4ccc5c(oc6ccc7oc8ccccc8c7c65)c4-c4cccc5c4sc4ccccc45)cc3)cc21 ; c1ccc(-c2cc(N(c3ccccc3)c3ccccc3)cc3c2oc2ccc4oc5ccccc5c4c23)cc1 ; c1ccc(-c2ccc(N(c3ccccc3)c3cc(-c4ccccc4)c4oc5ccc6oc7ccccc7c6c5c4c3)cc2)cc1]

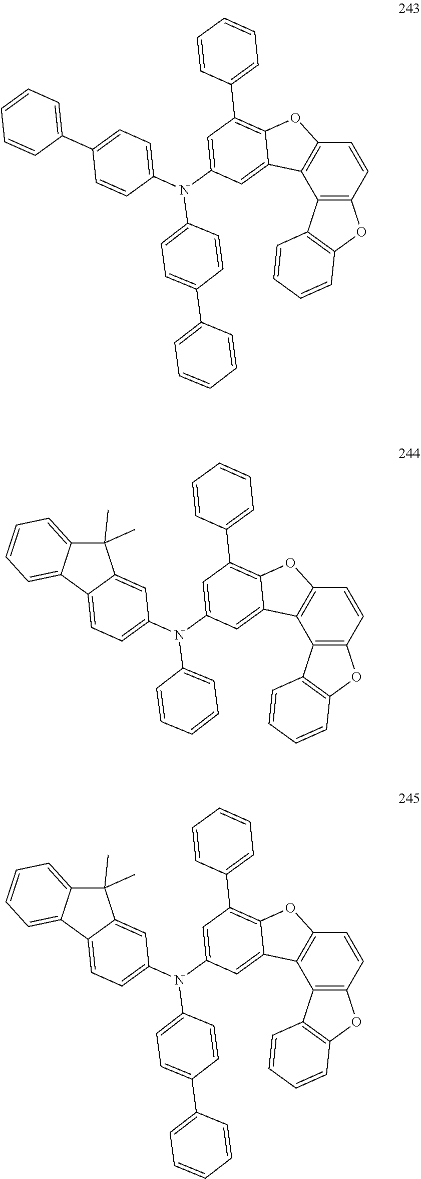 [SMILES: c1ccc(-c2ccc(N(c3ccc(-c4ccccc4)cc3)c3cc(-c4ccccc4)c4oc5ccc6oc7ccccc7c6c5c4c3)cc2)cc1 ; CC1(C)c2ccccc2-c2ccc(N(c3ccccc3)c3cc(-c4ccccc4)c4oc5ccc6oc7ccccc7c6c5c4c3)cc21 ; CC1(C)c2ccccc2-c2ccc(N(c3ccc(-c4ccccc4)cc3)c3cc(-c4ccccc4)c4oc5ccc6oc7ccccc7c6c5c4c3)cc21]

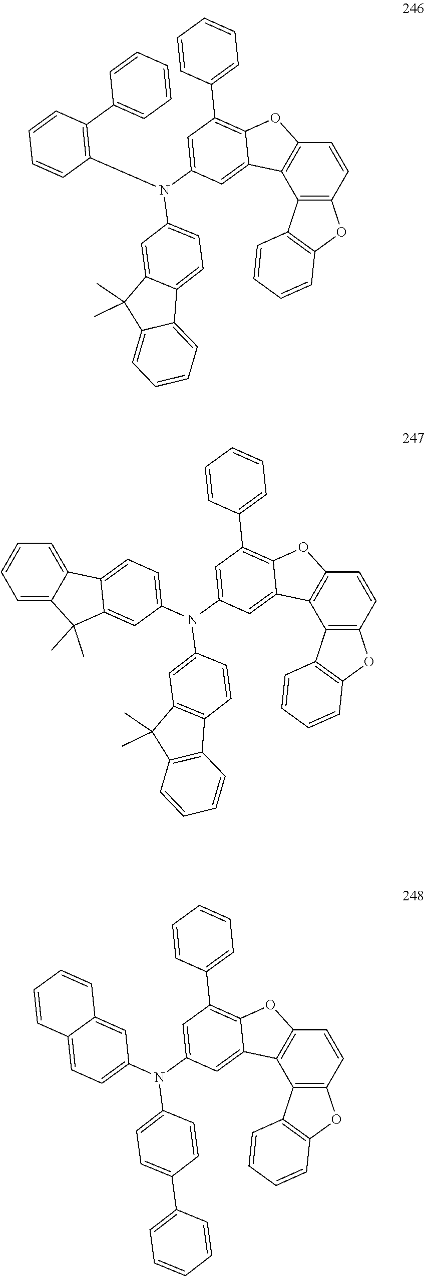 [SMILES: CC1(C)c2ccccc2-c2ccc(N(c3cc(-c4ccccc4)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4ccccc4)c4oc5ccc6oc7ccccc7c6c5c4c3)cc21 ; c1ccc(-c2ccc(N(c3ccc4ccccc4c3)c3cc(-c4ccccc4)c4oc5ccc6oc7ccccc7c6c5c4c3)cc2)cc1]

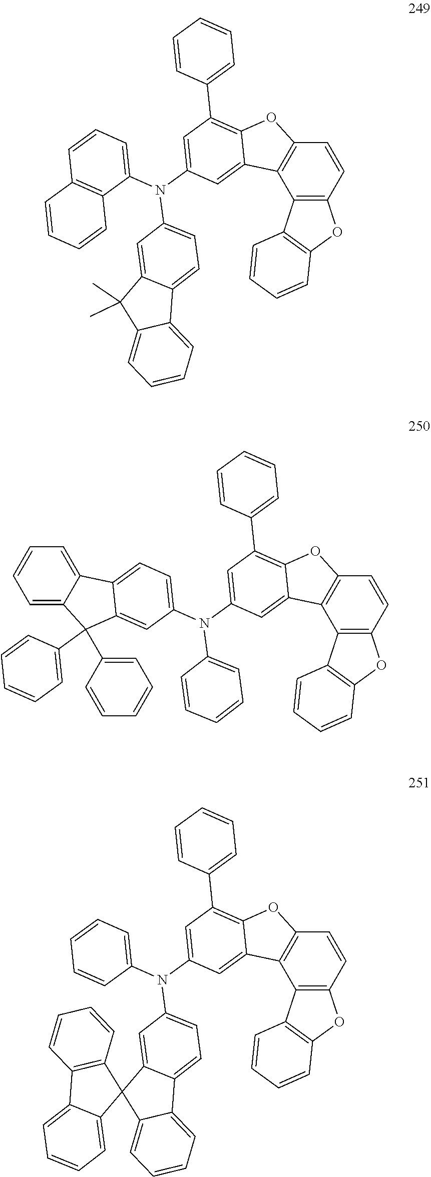 [SMILES: CC1(C)c2ccccc2-c2ccc(N(c3cc(-c4ccccc4)c4oc5ccc6oc7ccccc7c6c5c4c3)c3cccc4ccccc34)cc21 ; c1ccc(-c2cc(N(c3ccccc3)c3ccc4c(c3)C(c3ccccc3)(c3ccccc3)c3ccccc3-4)cc3c2oc2ccc4oc5ccccc5c4c23)cc1 ; c1ccc(-c2cc(N(c3ccccc3)c3ccc4c(c3)C3(c5ccccc5-c5ccccc53)c3ccccc3-4)cc3c2oc2ccc4oc5ccccc5c4c23)cc1]

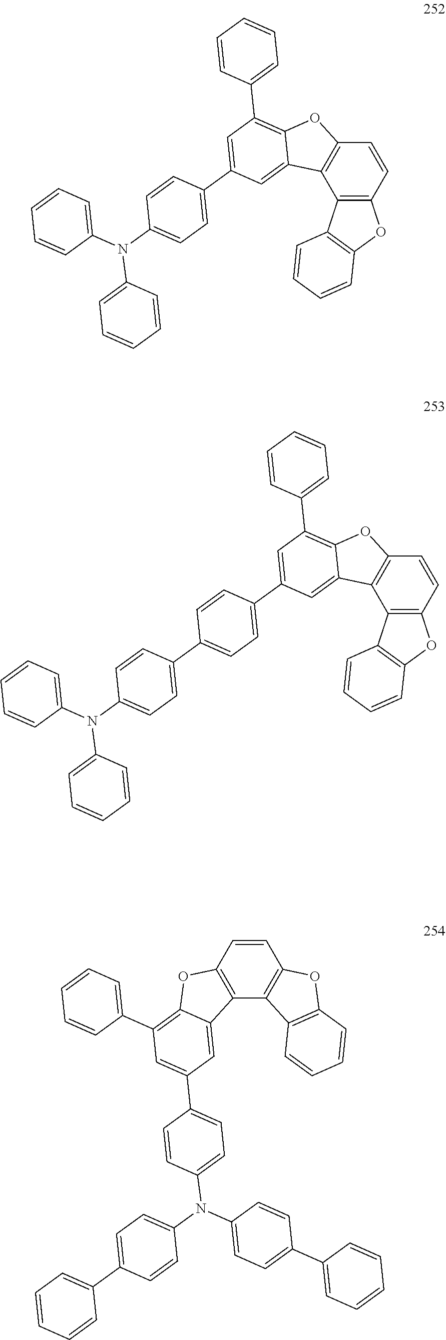 [SMILES: c1ccc(-c2cc(-c3ccc(N(c4ccccc4)c4ccccc4)cc3)cc3c2oc2ccc4oc5ccccc5c4c23)cc1 ; c1ccc(-c2cc(-c3ccc(-c4ccc(N(c5ccccc5)c5ccccc5)cc4)cc3)cc3c2oc2ccc4oc5ccccc5c4c23)cc1 ; c1ccc(-c2ccc(N(c3ccc(-c4ccccc4)cc3)c3ccc(-c4cc(-c5ccccc5)c5oc6ccc7oc8ccccc8c7c6c5c4)cc3)cc2)cc1]

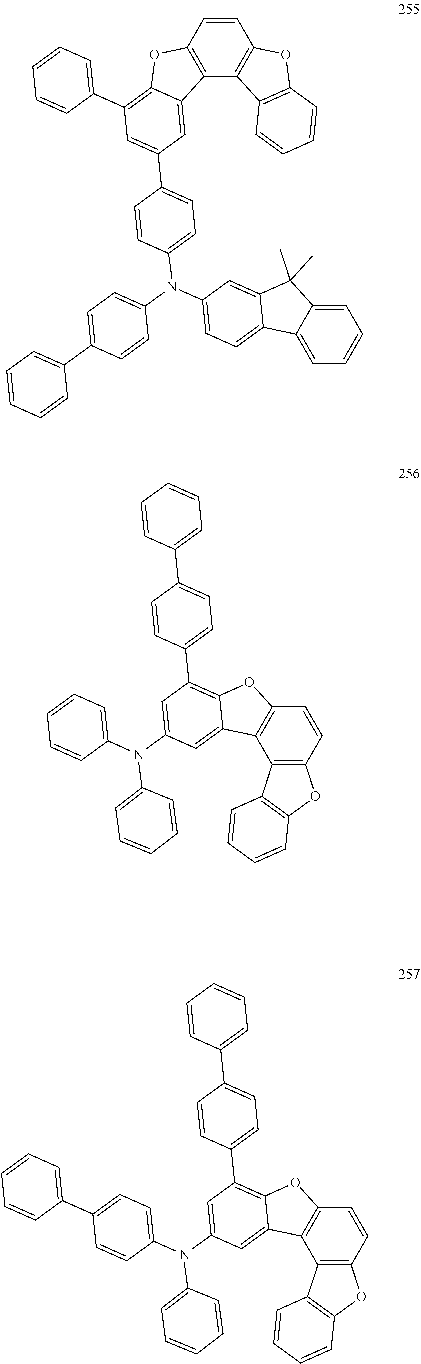 [SMILES: CC1(C)c2ccccc2-c2ccc(N(c3ccc(-c4ccccc4)cc3)c3ccc(-c4cc(-c5ccccc5)c5oc6ccc7oc8ccccc8c7c6c5c4)cc3)cc21 ; c1ccc(-c2ccc(-c3cc(N(c4ccccc4)c4ccccc4)cc4c3oc3ccc5oc6ccccc6c5c34)cc2)cc1 ; c1ccc(-c2ccc(-c3cc(N(c4ccccc4)c4ccc(-c5ccccc5)cc4)cc4c3oc3ccc5oc6ccccc6c5c34)cc2)cc1]

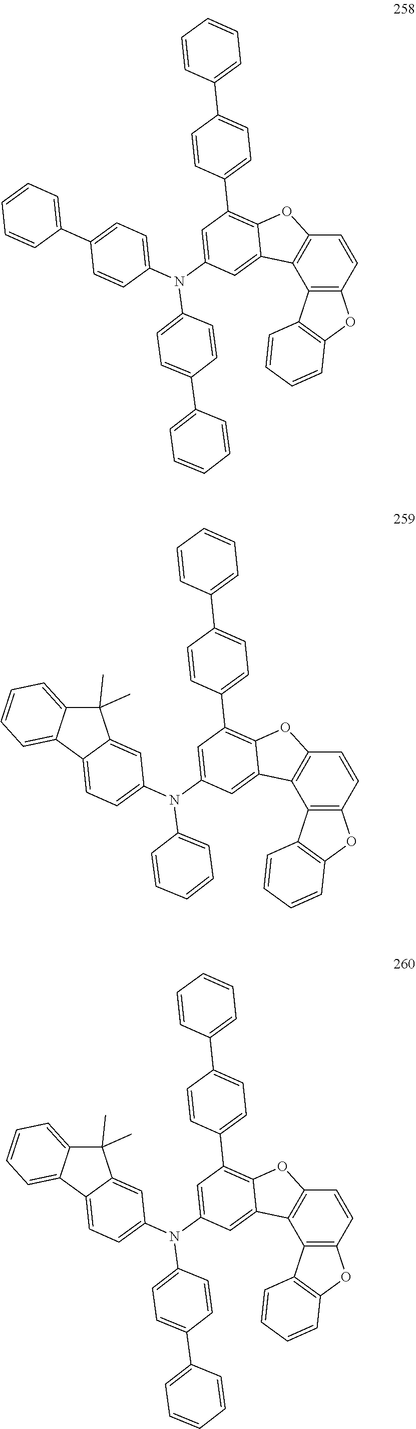 [SMILES: c1ccc(-c2ccc(-c3cc(N(c4ccc(-c5ccccc5)cc4)c4ccc(-c5ccccc5)cc4)cc4c3oc3ccc5oc6ccccc6c5c34)cc2)cc1 ; CC1(C)c2ccccc2-c2ccc(N(c3ccccc3)c3cc(-c4ccc(-c5ccccc5)cc4)c4oc5ccc6oc7ccccc7c6c5c4c3)cc21 ; CC1(C)c2ccccc2-c2ccc(N(c3ccc(-c4ccccc4)cc3)c3cc(-c4ccc(-c5ccccc5)cc4)c4oc5ccc6oc7ccccc7c6c5c4c3)cc21]

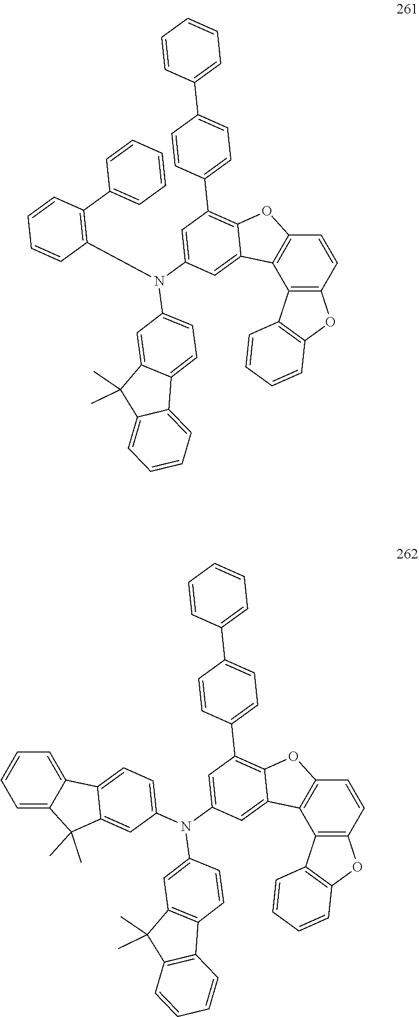 [SMILES: CC1(C)c2ccccc2-c2ccc(N(c3cc(-c4ccc(-c5ccccc5)cc4)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4ccc(-c5ccccc5)cc4)c4oc5ccc6oc7ccccc7c6c5c4c3)cc21]

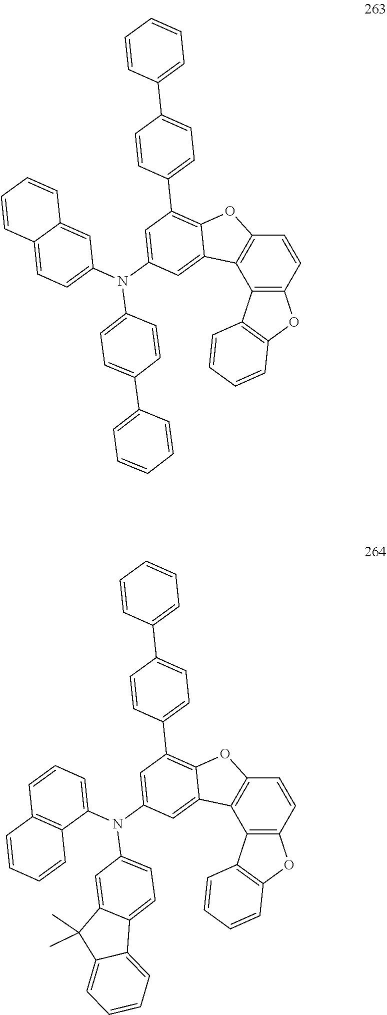 [SMILES: c1ccc(-c2ccc(-c3cc(N(c4ccc(-c5ccccc5)cc4)c4ccc5ccccc5c4)cc4c3oc3ccc5oc6ccccc6c5c34)cc2)cc1 ; CC1(C)c2ccccc2-c2ccc(N(c3cc(-c4ccc(-c5ccccc5)cc4)c4oc5ccc6oc7ccccc7c6c5c4c3)c3cccc4ccccc34)cc21]

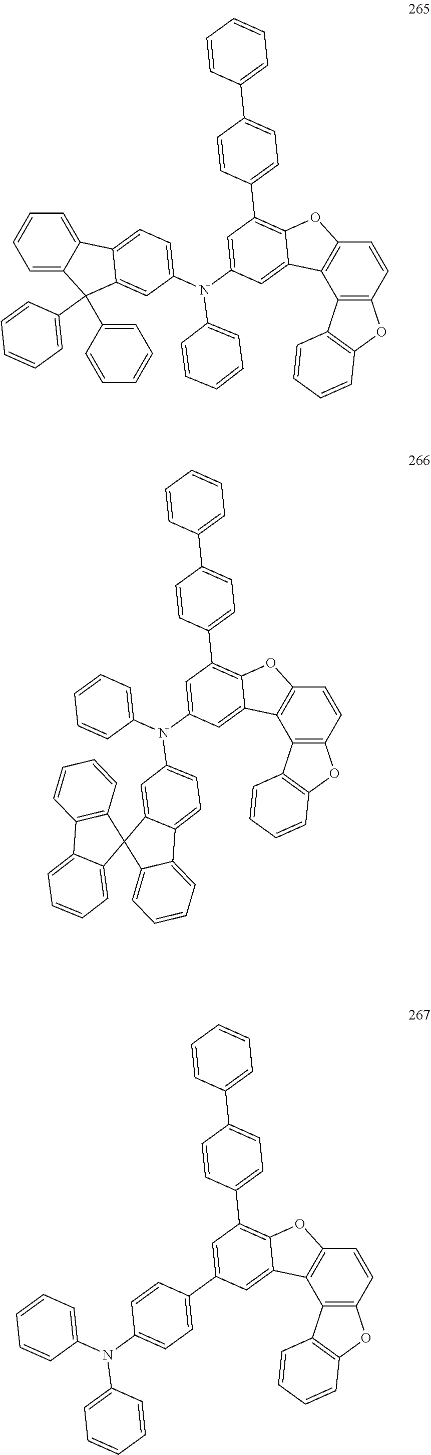 [SMILES: c1ccc(-c2ccc(-c3cc(N(c4ccccc4)c4ccc5c(c4)C(c4ccccc4)(c4ccccc4)c4ccccc4-5)cc4c3oc3ccc5oc6ccccc6c5c34)cc2)cc1 ; c1ccc(-c2ccc(-c3cc(N(c4ccccc4)c4ccc5c(c4)C4(c6ccccc6-c6ccccc64)c4ccccc4-5)cc4c3oc3ccc5oc6ccccc6c5c34)cc2)cc1 ; c1ccc(-c2ccc(-c3cc(-c4ccc(N(c5ccccc5)c5ccccc5)cc4)cc4c3oc3ccc5oc6ccccc6c5c34)cc2)cc1]

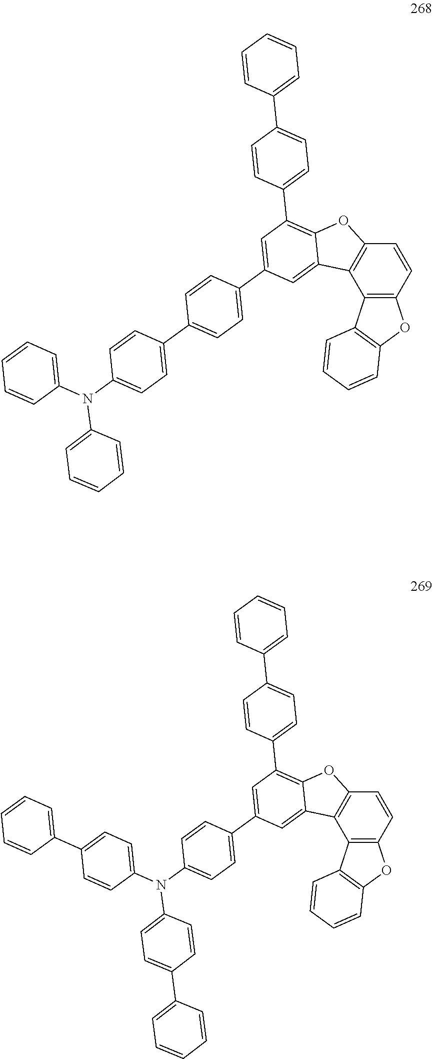 [SMILES: c1ccc(-c2ccc(-c3cc(-c4ccc(-c5ccc(N(c6ccccc6)c6ccccc6)cc5)cc4)cc4c3oc3ccc5oc6ccccc6c5c34)cc2)cc1 ; c1ccc(-c2ccc(-c3cc(-c4ccc(N(c5ccc(-c6ccccc6)cc5)c5ccc(-c6ccccc6)cc5)cc4)cc4c3oc3ccc5oc6ccccc6c5c34)cc2)cc1]

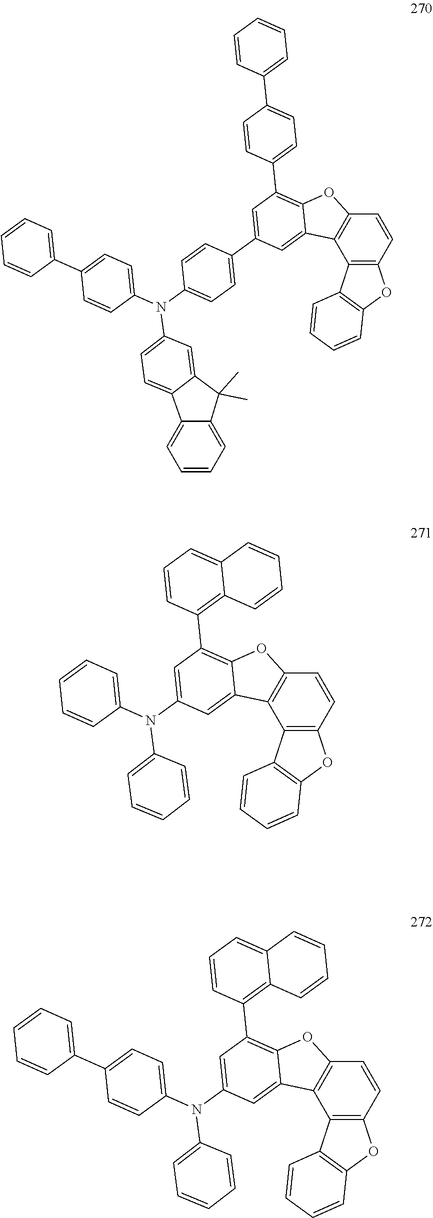 [SMILES: CC1(C)c2ccccc2-c2ccc(N(c3ccc(-c4ccccc4)cc3)c3ccc(-c4cc(-c5ccc(-c6ccccc6)cc5)c5oc6ccc7oc8ccccc8c7c6c5c4)cc3)cc21 ; c1ccc(N(c2ccccc2)c2cc(-c3cccc4ccccc34)c3oc4ccc5oc6ccccc6c5c4c3c2)cc1 ; c1ccc(-c2ccc(N(c3ccccc3)c3cc(-c4cccc5ccccc45)c4oc5ccc6oc7ccccc7c6c5c4c3)cc2)cc1]

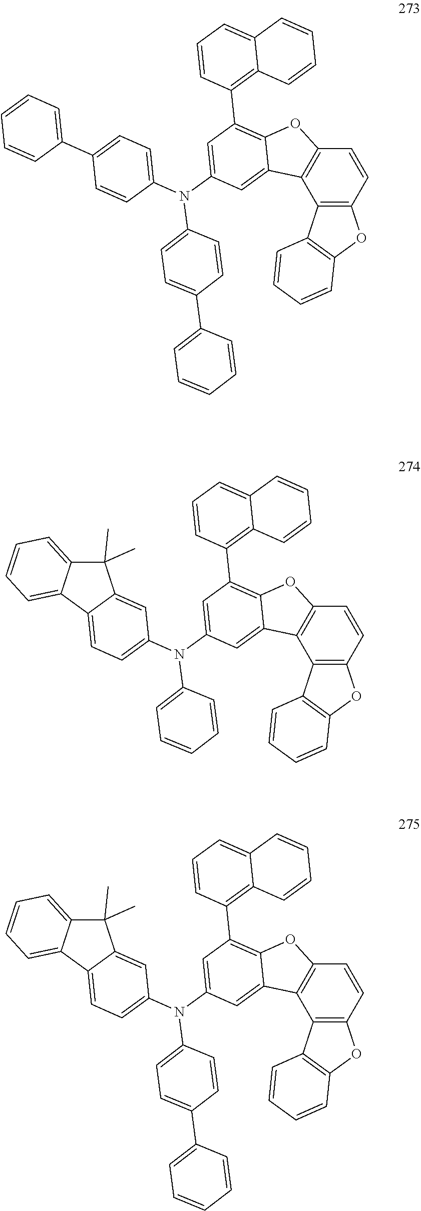 [SMILES: c1ccc(-c2ccc(N(c3ccc(-c4ccccc4)cc3)c3cc(-c4cccc5ccccc45)c4oc5ccc6oc7ccccc7c6c5c4c3)cc2)cc1 ; CC1(C)c2ccccc2-c2ccc(N(c3ccccc3)c3cc(-c4cccc5ccccc45)c4oc5ccc6oc7ccccc7c6c5c4c3)cc21 ; CC1(C)c2ccccc2-c2ccc(N(c3ccc(-c4ccccc4)cc3)c3cc(-c4cccc5ccccc45)c4oc5ccc6oc7ccccc7c6c5c4c3)cc21]

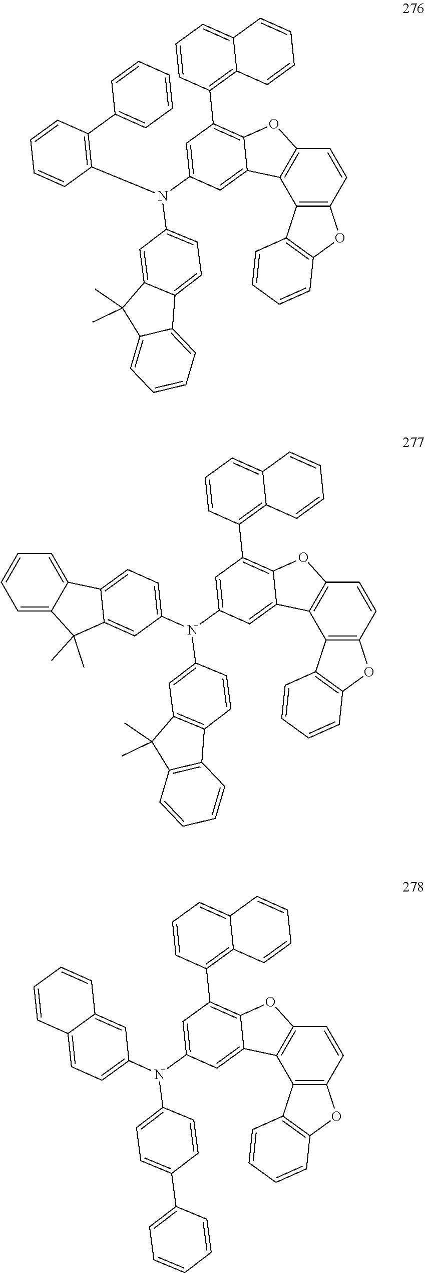 [SMILES: CC1(C)c2ccccc2-c2ccc(N(c3cc(-c4cccc5ccccc45)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4cccc5ccccc45)c4oc5ccc6oc7ccccc7c6c5c4c3)cc21 ; c1ccc(-c2ccc(N(c3ccc4ccccc4c3)c3cc(-c4cccc5ccccc45)c4oc5ccc6oc7ccccc7c6c5c4c3)cc2)cc1]

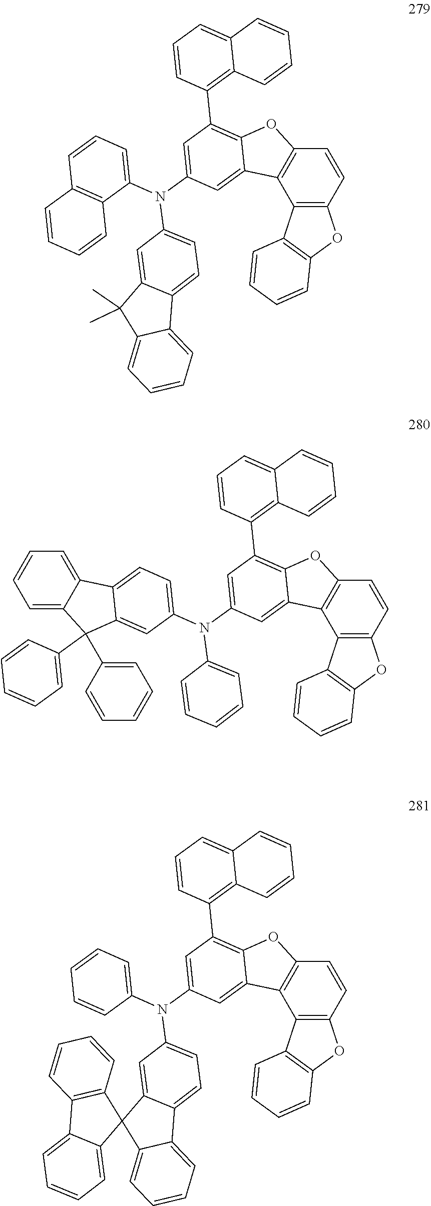 [SMILES: CC1(C)c2ccccc2-c2ccc(N(c3cc(-c4cccc5ccccc45)c4oc5ccc6oc7ccccc7c6c5c4c3)c3cccc4ccccc34)cc21 ; c1ccc(N(c2ccc3c(c2)C(c2ccccc2)(c2ccccc2)c2ccccc2-3)c2cc(-c3cccc4ccccc34)c3oc4ccc5oc6ccccc6c5c4c3c2)cc1 ; c1ccc(N(c2ccc3c(c2)C2(c4ccccc4-c4ccccc42)c2ccccc2-3)c2cc(-c3cccc4ccccc34)c3oc4ccc5oc6ccccc6c5c4c3c2)cc1]

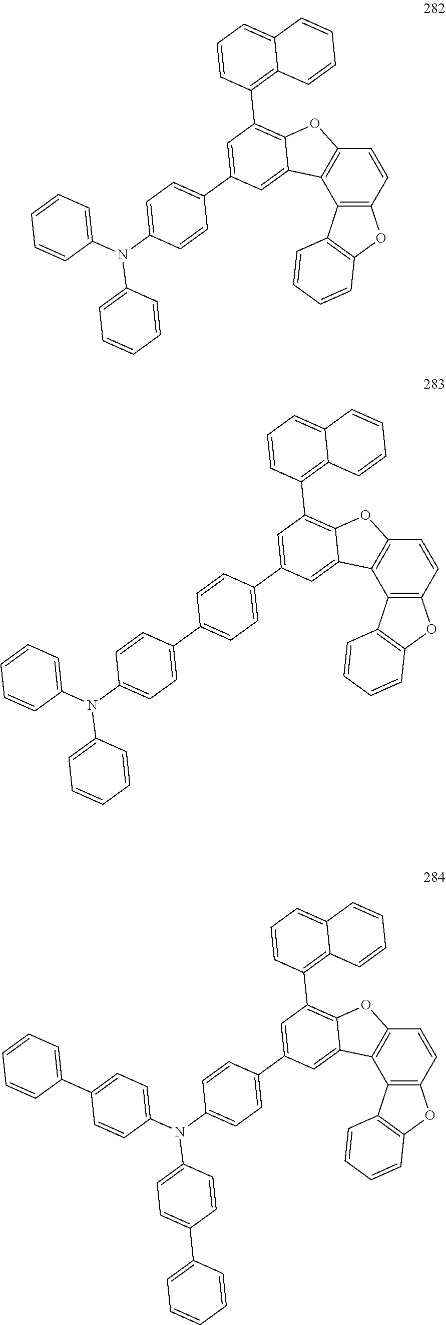 [SMILES: c1ccc(N(c2ccccc2)c2ccc(-c3cc(-c4cccc5ccccc45)c4oc5ccc6oc7ccccc7c6c5c4c3)cc2)cc1 ; c1ccc(N(c2ccccc2)c2ccc(-c3ccc(-c4cc(-c5cccc6ccccc56)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5cccc6ccccc56)c5oc6ccc7oc8ccccc8c7c6c5c4)cc3)cc2)cc1]

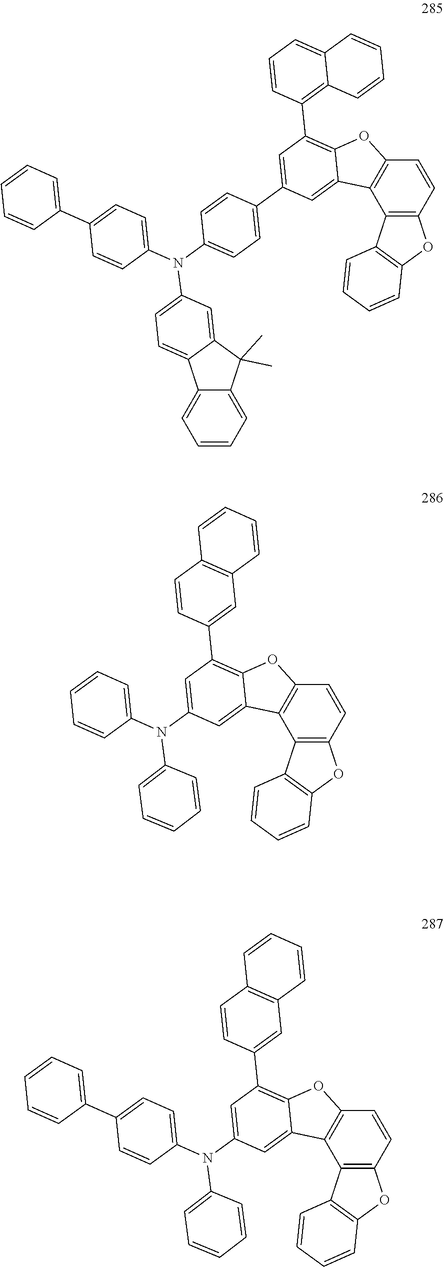 [SMILES: CC1(C)c2ccccc2-c2ccc(N(c3ccc(-c4ccccc4)cc3)c3ccc(-c4cc(-c5cccc6ccccc56)c5oc6ccc7oc8ccccc8c7c6c5c4)cc3)cc21 ; c1ccc(N(c2ccccc2)c2cc(-c3ccc4ccccc4c3)c3oc4ccc5oc6ccccc6c5c4c3c2)cc1 ; c1ccc(-c2ccc(N(c3ccccc3)c3cc(-c4ccc5ccccc5c4)c4oc5ccc6oc7ccccc7c6c5c4c3)cc2)cc1]

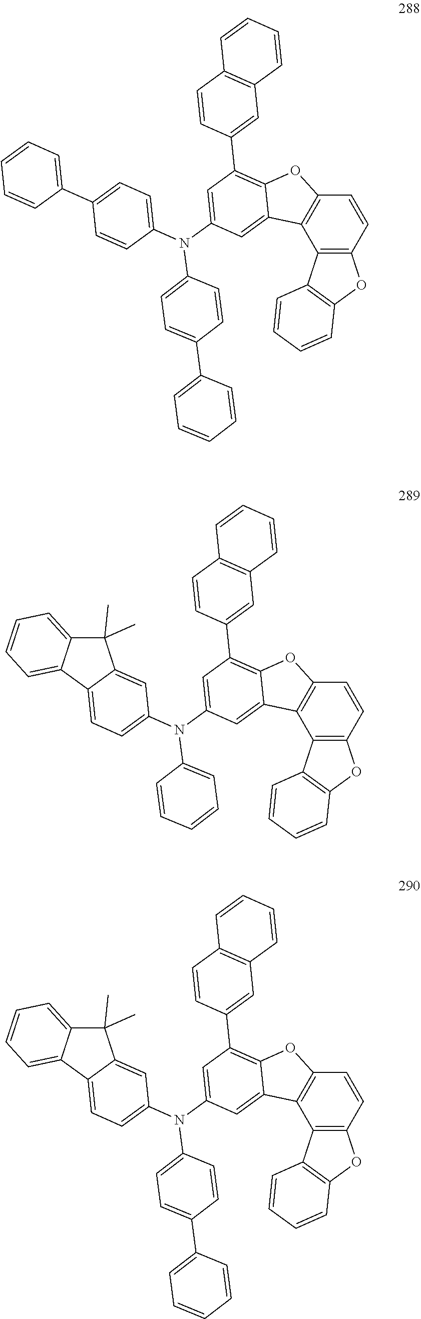 [SMILES: c1ccc(-c2ccc(N(c3ccc(-c4ccccc4)cc3)c3cc(-c4ccc5ccccc5c4)c4oc5ccc6oc7ccccc7c6c5c4c3)cc2)cc1 ; CC1(C)c2ccccc2-c2ccc(N(c3ccccc3)c3cc(-c4ccc5ccccc5c4)c4oc5ccc6oc7ccccc7c6c5c4c3)cc21 ; CC1(C)c2ccccc2-c2ccc(N(c3ccc(-c4ccccc4)cc3)c3cc(-c4ccc5ccccc5c4)c4oc5ccc6oc7ccccc7c6c5c4c3)cc21]

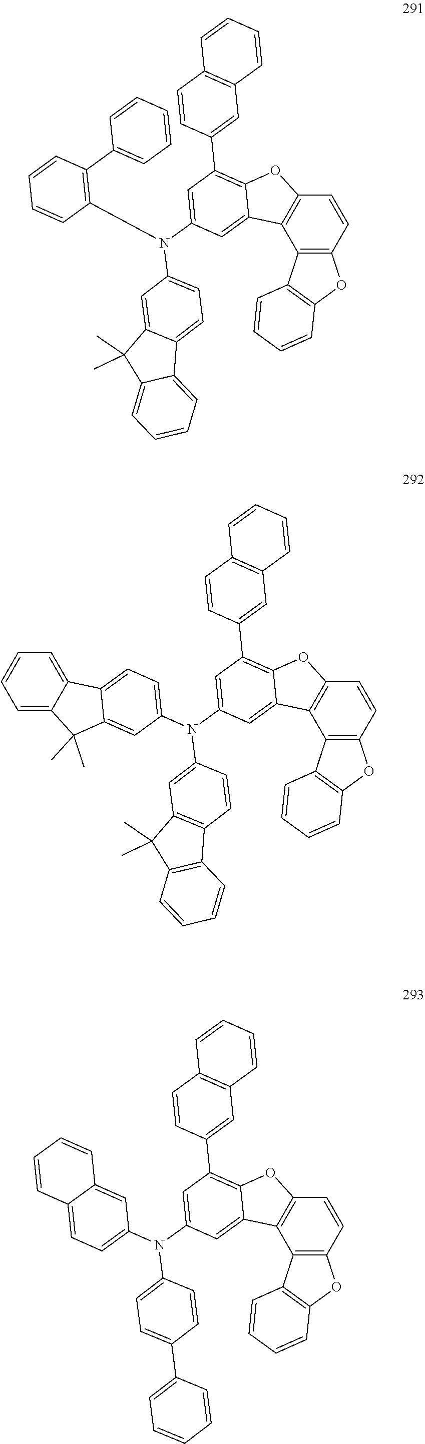 [SMILES: CC1(C)c2ccccc2-c2ccc(N(c3cc(-c4ccc5ccccc5c4)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4ccc5ccccc5c4)c4oc5ccc6oc7ccccc7c6c5c4c3)cc21 ; c1ccc(-c2ccc(N(c3ccc4ccccc4c3)c3cc(-c4ccc5ccccc5c4)c4oc5ccc6oc7ccccc7c6c5c4c3)cc2)cc1]

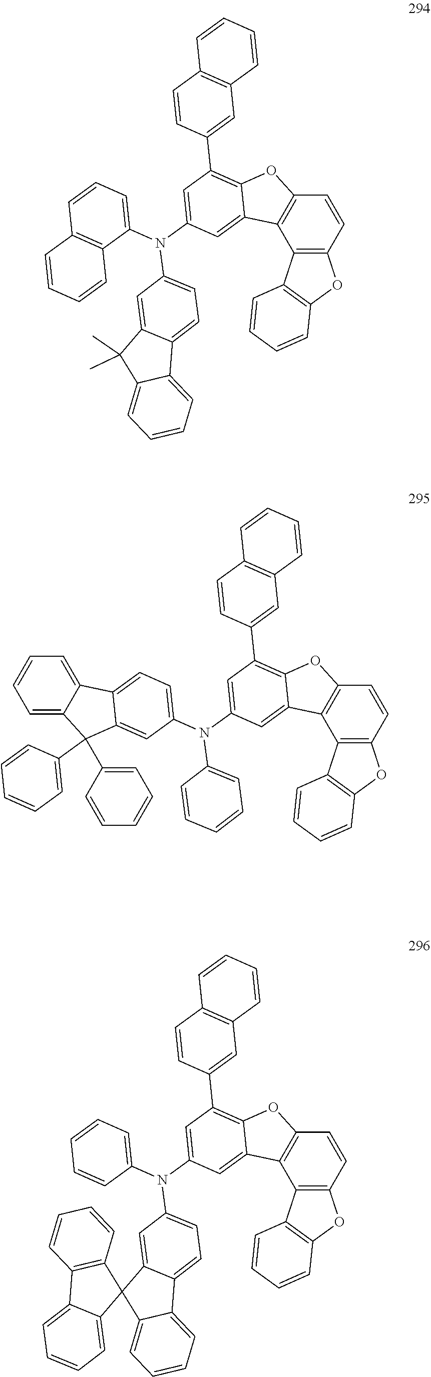 [SMILES: CC1(C)c2ccccc2-c2ccc(N(c3cc(-c4ccc5ccccc5c4)c4oc5ccc6oc7ccccc7c6c5c4c3)c3cccc4ccccc34)cc21 ; c1ccc(N(c2ccc3c(c2)C(c2ccccc2)(c2ccccc2)c2ccccc2-3)c2cc(-c3ccc4ccccc4c3)c3oc4ccc5oc6ccccc6c5c4c3c2)cc1 ; c1ccc(N(c2ccc3c(c2)C2(c4ccccc4-c4ccccc42)c2ccccc2-3)c2cc(-c3ccc4ccccc4c3)c3oc4ccc5oc6ccccc6c5c4c3c2)cc1]

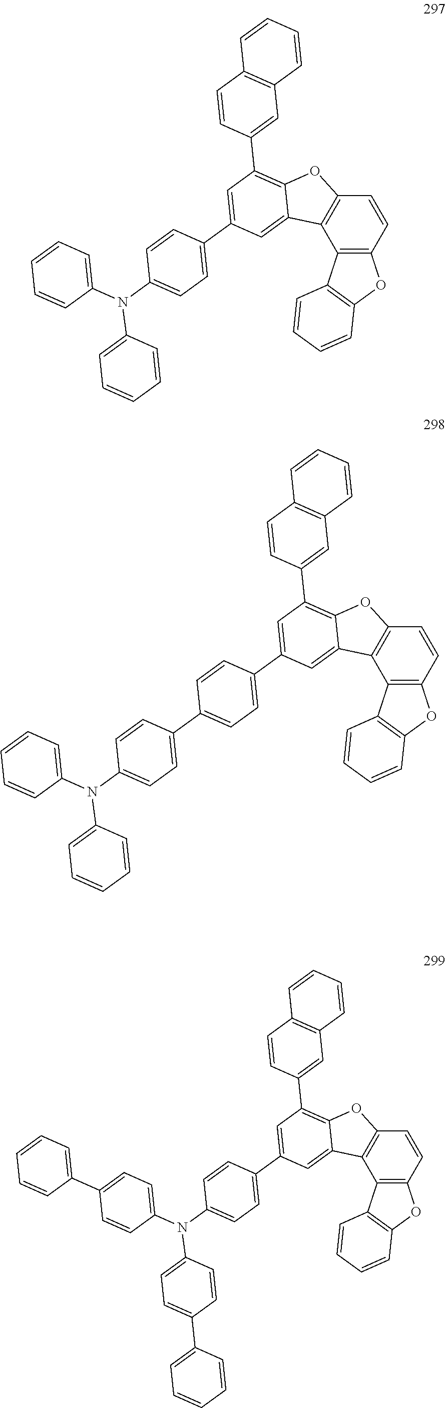 [SMILES: c1ccc(N(c2ccccc2)c2ccc(-c3cc(-c4ccc5ccccc5c4)c4oc5ccc6oc7ccccc7c6c5c4c3)cc2)cc1 ; c1ccc(N(c2ccccc2)c2ccc(-c3ccc(-c4cc(-c5ccc6ccccc6c5)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5ccc6ccccc6c5)c5oc6ccc7oc8ccccc8c7c6c5c4)cc3)cc2)cc1]

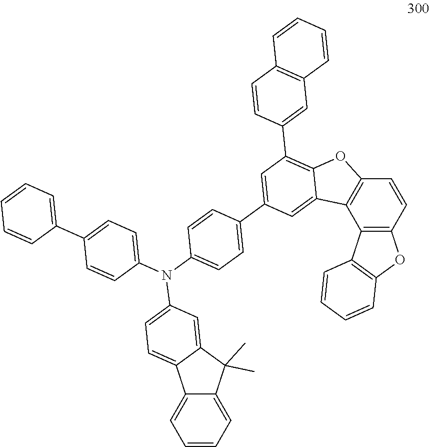 [SMILES: CC1(C)c2ccccc2-c2ccc(N(c3ccc(-c4ccccc4)cc3)c3ccc(-c4cc(-c5ccc6ccccc6c5)c5oc6ccc7oc8ccccc8c7c6c5c4)cc3)cc21]

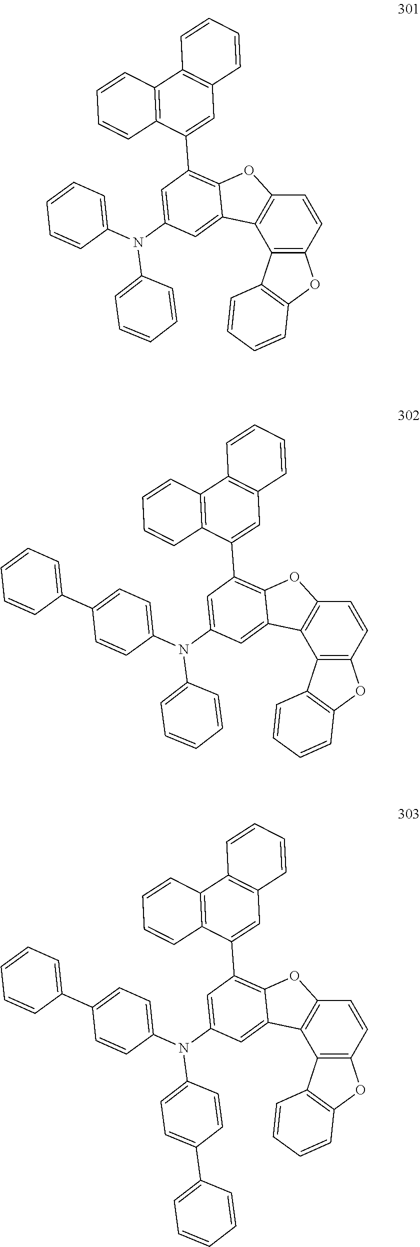 [SMILES: c1ccc(N(c2ccccc2)c2cc(-c3cc4ccccc4c4ccccc34)c3oc4ccc5oc6ccccc6c5c4c3c2)cc1 ; c1ccc(-c2ccc(N(c3ccccc3)c3cc(-c4cc5ccccc5c5ccccc45)c4oc5ccc6oc7ccccc7c6c5c4c3)cc2)cc1 ; c1ccc(-c2ccc(N(c3ccc(-c4ccccc4)cc3)c3cc(-c4cc5ccccc5c5ccccc45)c4oc5ccc6oc7ccccc7c6c5c4c3)cc2)cc1]

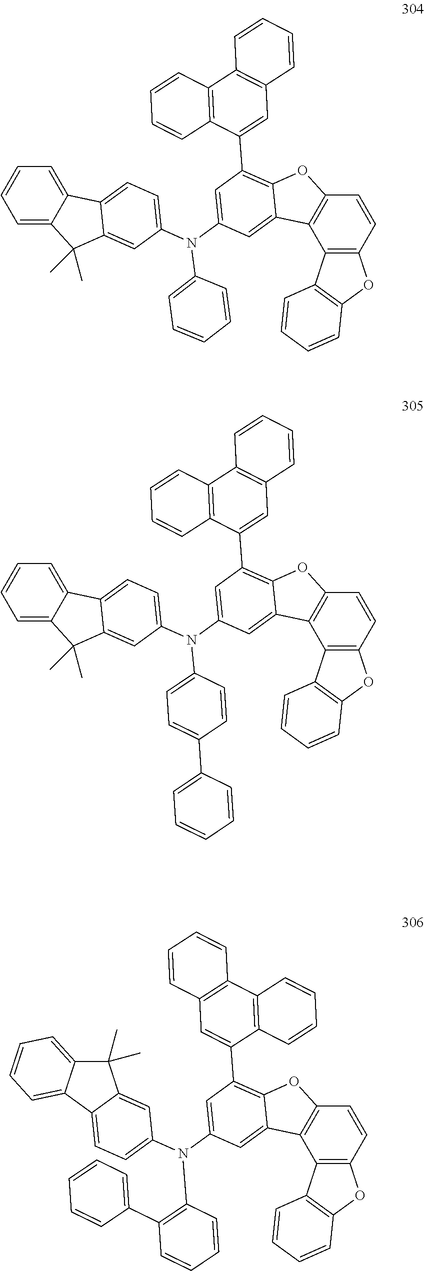 [SMILES: CC1(C)c2ccccc2-c2ccc(N(c3ccccc3)c3cc(-c4cc5ccccc5c5ccccc45)c4oc5ccc6oc7ccccc7c6c5c4c3)cc21 ; CC1(C)c2ccccc2-c2ccc(N(c3ccc(-c4ccccc4)cc3)c3cc(-c4cc5ccccc5c5ccccc45)c4oc5ccc6oc7ccccc7c6c5c4c3)cc21 ; CC1(C)c2ccccc2-c2ccc(N(c3cc(-c4cc5ccccc5c5ccccc45)c4oc5ccc6oc7ccccc7c6c5c4c3)c3ccccc3-c3ccccc3)cc21]

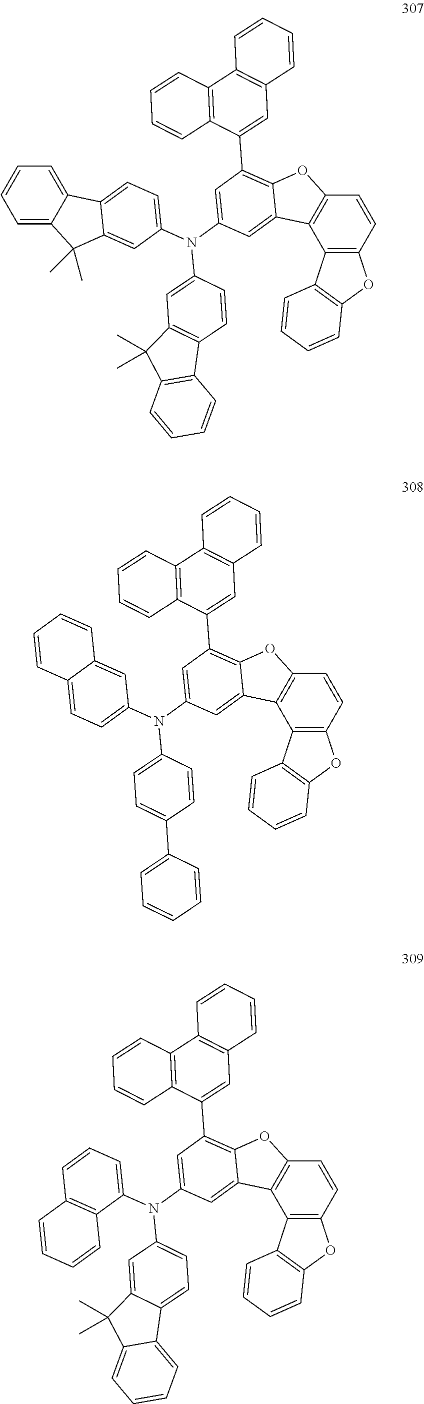 [SMILES: CC1(C)c2ccccc2-c2ccc(N(c3ccc4c(c3)C(C)(C)c3ccccc3-4)c3cc(-c4cc5ccccc5c5ccccc45)c4oc5ccc6oc7ccccc7c6c5c4c3)cc21 ; c1ccc(-c2ccc(N(c3ccc4ccccc4c3)c3cc(-c4cc5ccccc5c5ccccc45)c4oc5ccc6oc7ccccc7c6c5c4c3)cc2)cc1 ; CC1(C)c2ccccc2-c2ccc(N(c3cc(-c4cc5ccccc5c5ccccc45)c4oc5ccc6oc7ccccc7c6c5c4c3)c3cccc4ccccc34)cc21]

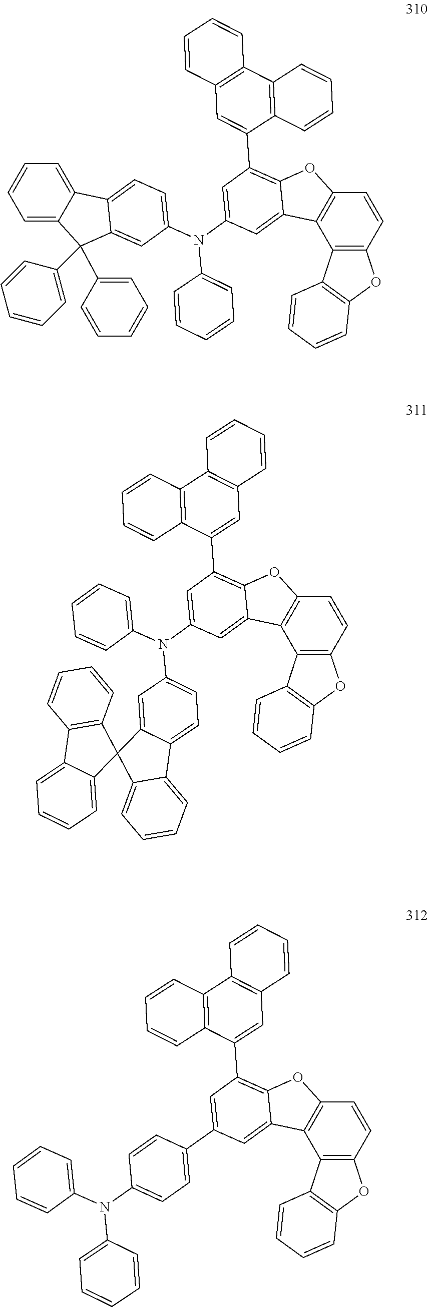 [SMILES: c1ccc(N(c2ccc3c(c2)C(c2ccccc2)(c2ccccc2)c2ccccc2-3)c2cc(-c3cc4ccccc4c4ccccc34)c3oc4ccc5oc6ccccc6c5c4c3c2)cc1 ; c1ccc(N(c2ccc3c(c2)C2(c4ccccc4-c4ccccc42)c2ccccc2-3)c2cc(-c3cc4ccccc4c4ccccc34)c3oc4ccc5oc6ccccc6c5c4c3c2)cc1 ; c1ccc(N(c2ccccc2)c2ccc(-c3cc(-c4cc5ccccc5c5ccccc45)c4oc5ccc6oc7ccccc7c6c5c4c3)cc2)cc1]

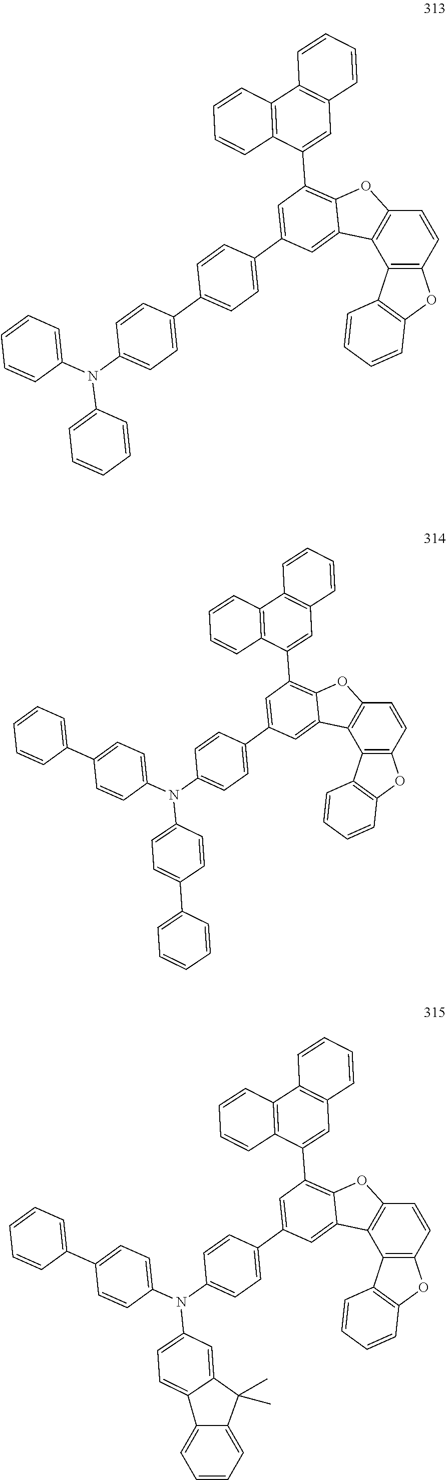 [SMILES: c1ccc(N(c2ccccc2)c2ccc(-c3ccc(-c4cc(-c5cc6ccccc6c6ccccc56)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5cc6ccccc6c6ccccc56)c5oc6ccc7oc8ccccc8c7c6c5c4)cc3)cc2)cc1 ; CC1(C)c2ccccc2-c2ccc(N(c3ccc(-c4ccccc4)cc3)c3ccc(-c4cc(-c5cc6ccccc6c6ccccc56)c5oc6ccc7oc8ccccc8c7c6c5c4)cc3)cc21]

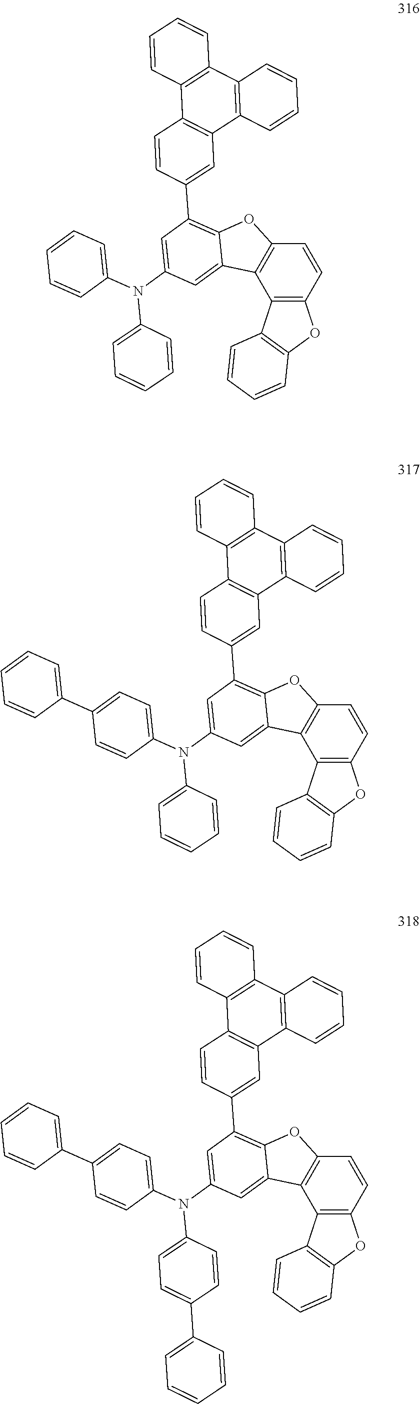 [SMILES: c1ccc(N(c2ccccc2)c2cc(-c3ccc4c5ccccc5c5ccccc5c4c3)c3oc4ccc5oc6ccccc6c5c4c3c2)cc1 ; c1ccc(-c2ccc(N(c3ccccc3)c3cc(-c4ccc5c6ccccc6c6ccccc6c5c4)c4oc5ccc6oc7ccccc7c6c5c4c3)cc2)cc1 ; c1ccc(-c2ccc(N(c3ccc(-c4ccccc4)cc3)c3cc(-c4ccc5c6ccccc6c6ccccc6c5c4)c4oc5ccc6oc7ccccc7c6c5c4c3)cc2)cc1]

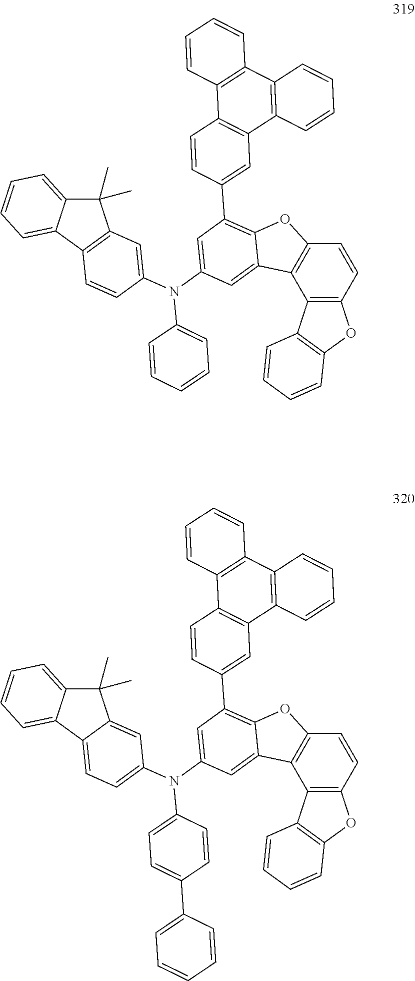 [SMILES: CC1(C)c2ccccc2-c2ccc(N(c3ccccc3)c3cc(-c4ccc5c6ccccc6c6ccccc6c5c4)c4oc5ccc6oc7ccccc7c6c5c4c3)cc21 ; CC1(C)c2ccccc2-c2ccc(N(c3ccc(-c4ccccc4)cc3)c3cc(-c4ccc5c6ccccc6c6ccccc6c5c4)c4oc5ccc6oc7ccccc7c6c5c4c3)cc21]

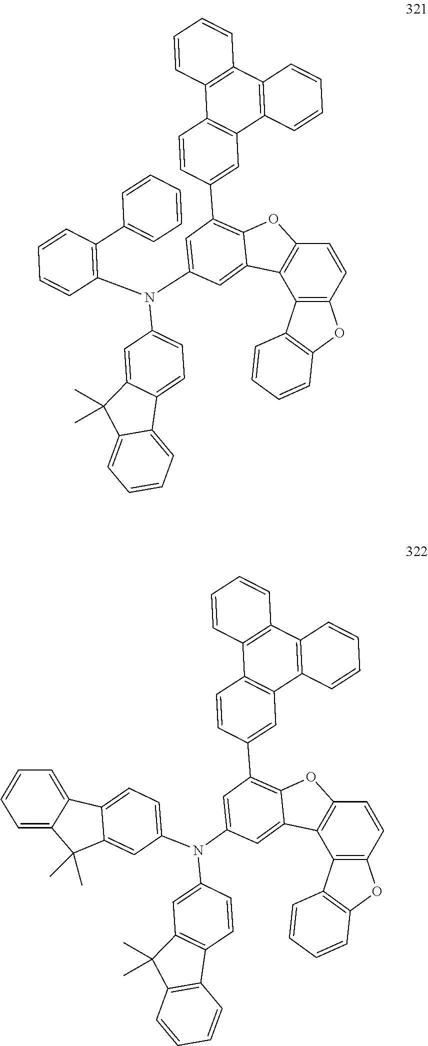 [SMILES: CC1(C)c2ccccc2-c2ccc(N(c3cc(-c4ccc5c6ccccc6c6ccccc6c5c4)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4ccc5c6ccccc6c6ccccc6c5c4)c4oc5ccc6oc7ccccc7c6c5c4c3)cc21]

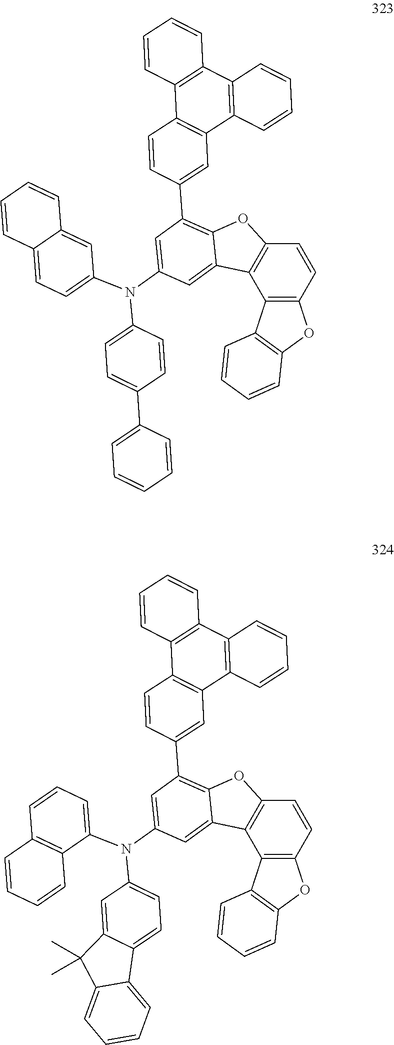 [SMILES: c1ccc(-c2ccc(N(c3ccc4ccccc4c3)c3cc(-c4ccc5c6ccccc6c6ccccc6c5c4)c4oc5ccc6oc7ccccc7c6c5c4c3)cc2)cc1 ; CC1(C)c2ccccc2-c2ccc(N(c3cc(-c4ccc5c6ccccc6c6ccccc6c5c4)c4oc5ccc6oc7ccccc7c6c5c4c3)c3cccc4ccccc34)cc21]

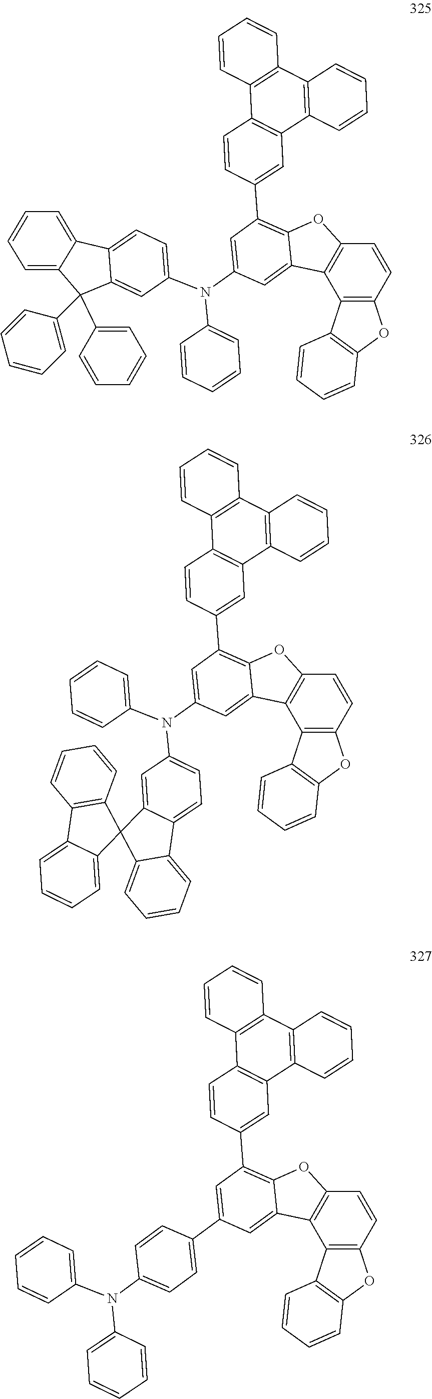 [SMILES: c1ccc(N(c2ccc3c(c2)C(c2ccccc2)(c2ccccc2)c2ccccc2-3)c2cc(-c3ccc4c5ccccc5c5ccccc5c4c3)c3oc4ccc5oc6ccccc6c5c4c3c2)cc1 ; c1ccc(N(c2ccc3c(c2)C2(c4ccccc4-c4ccccc42)c2ccccc2-3)c2cc(-c3ccc4c5ccccc5c5ccccc5c4c3)c3oc4ccc5oc6ccccc6c5c4c3c2)cc1 ; c1ccc(N(c2ccccc2)c2ccc(-c3cc(-c4ccc5c6ccccc6c6ccccc6c5c4)c4oc5ccc6oc7ccccc7c6c5c4c3)cc2)cc1]

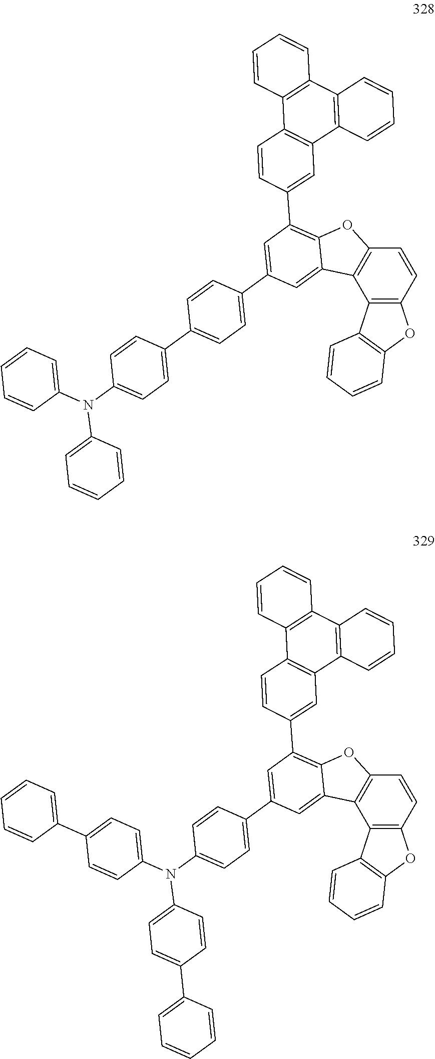 [SMILES: c1ccc(N(c2ccccc2)c2ccc(-c3ccc(-c4cc(-c5ccc6c7ccccc7c7ccccc7c6c5)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5ccc6c7ccccc7c7ccccc7c6c5)c5oc6ccc7oc8ccccc8c7c6c5c4)cc3)cc2)cc1]

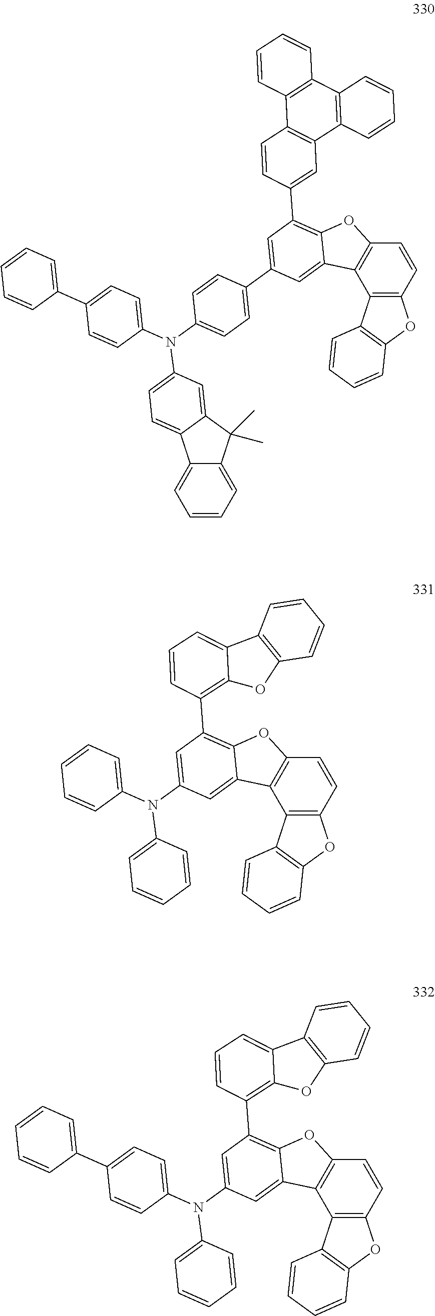 [SMILES: CC1(C)c2ccccc2-c2ccc(N(c3ccc(-c4ccccc4)cc3)c3ccc(-c4cc(-c5ccc6c7ccccc7c7ccccc7c6c5)c5oc6ccc7oc8ccccc8c7c6c5c4)cc3)cc21 ; c1ccc(N(c2ccccc2)c2cc(-c3cccc4c3oc3ccccc34)c3oc4ccc5oc6ccccc6c5c4c3c2)cc1 ; c1ccc(-c2ccc(N(c3ccccc3)c3cc(-c4cccc5c4oc4ccccc45)c4oc5ccc6oc7ccccc7c6c5c4c3)cc2)cc1]

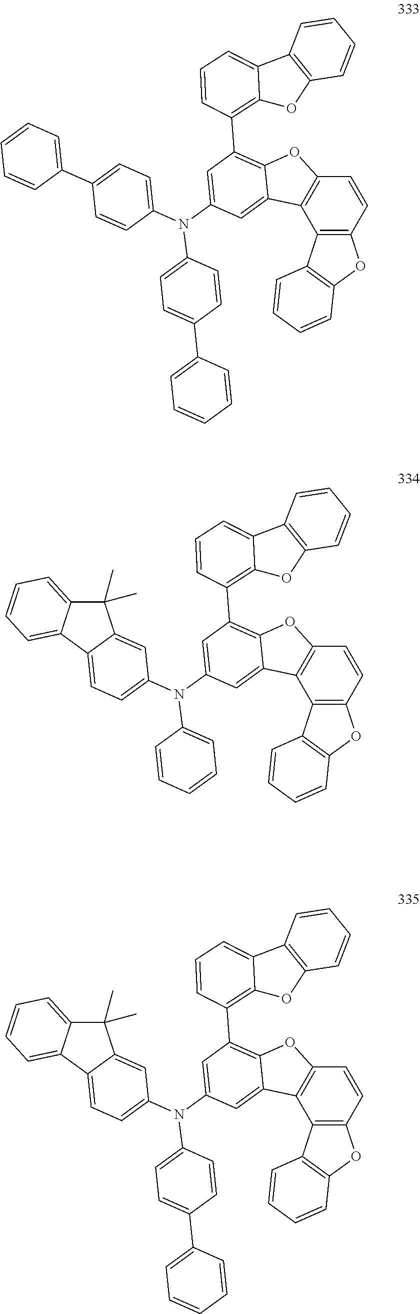 [SMILES: c1ccc(-c2ccc(N(c3ccc(-c4ccccc4)cc3)c3cc(-c4cccc5c4oc4ccccc45)c4oc5ccc6oc7ccccc7c6c5c4c3)cc2)cc1 ; CC1(C)c2ccccc2-c2ccc(N(c3ccccc3)c3cc(-c4cccc5c4oc4ccccc45)c4oc5ccc6oc7ccccc7c6c5c4c3)cc21 ; CC1(C)c2ccccc2-c2ccc(N(c3ccc(-c4ccccc4)cc3)c3cc(-c4cccc5c4oc4ccccc45)c4oc5ccc6oc7ccccc7c6c5c4c3)cc21]

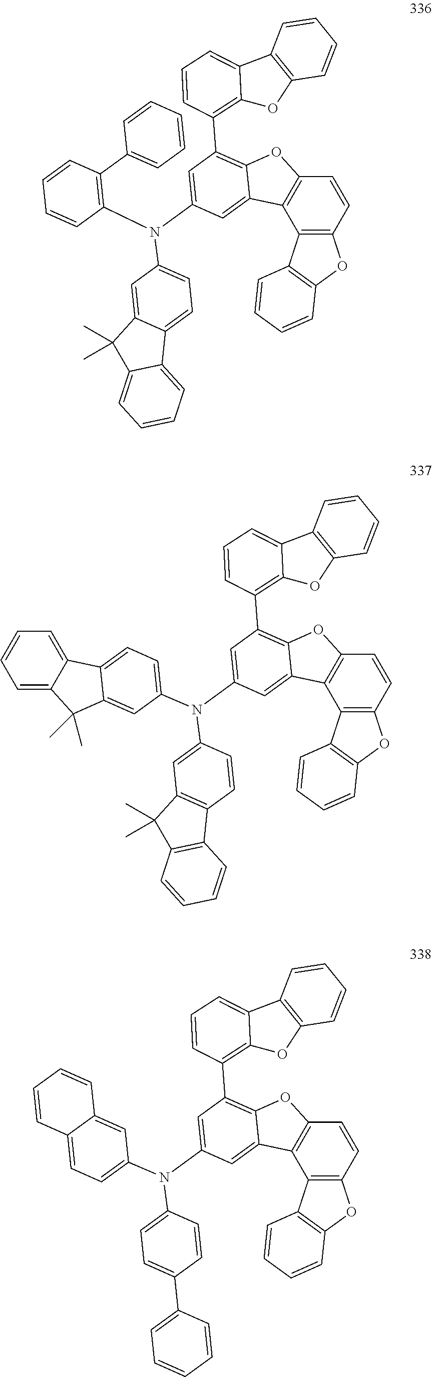 [SMILES: CC1(C)c2ccccc2-c2ccc(N(c3cc(-c4cccc5c4oc4ccccc45)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4cccc5c4oc4ccccc45)c4oc5ccc6oc7ccccc7c6c5c4c3)cc21 ; c1ccc(-c2ccc(N(c3ccc4ccccc4c3)c3cc(-c4cccc5c4oc4ccccc45)c4oc5ccc6oc7ccccc7c6c5c4c3)cc2)cc1]

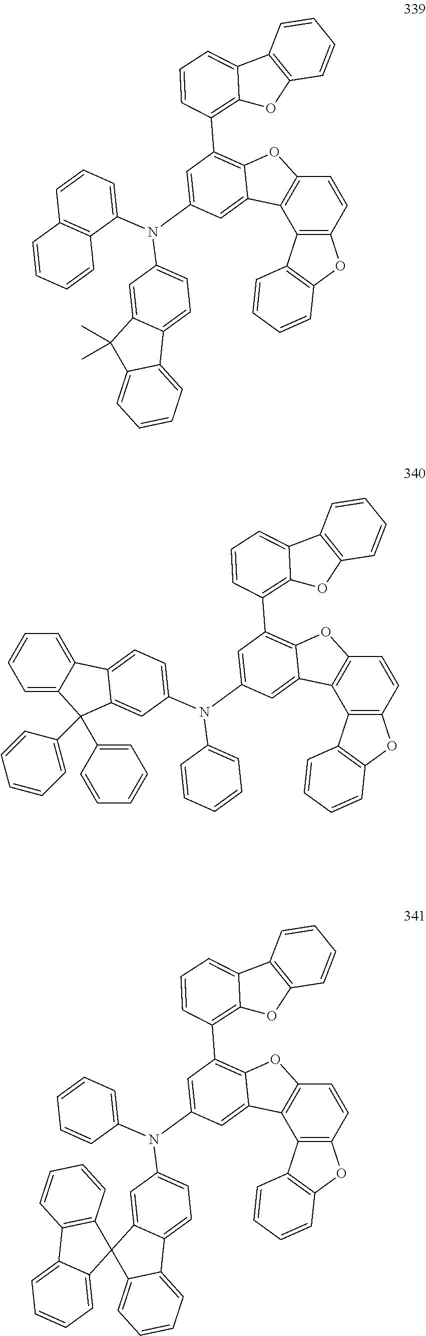 [SMILES: CC1(C)c2ccccc2-c2ccc(N(c3cc(-c4cccc5c4oc4ccccc45)c4oc5ccc6oc7ccccc7c6c5c4c3)c3cccc4ccccc34)cc21 ; c1ccc(N(c2ccc3c(c2)C(c2ccccc2)(c2ccccc2)c2ccccc2-3)c2cc(-c3cccc4c3oc3ccccc34)c3oc4ccc5oc6ccccc6c5c4c3c2)cc1 ; c1ccc(N(c2ccc3c(c2)C2(c4ccccc4-c4ccccc42)c2ccccc2-3)c2cc(-c3cccc4c3oc3ccccc34)c3oc4ccc5oc6ccccc6c5c4c3c2)cc1]

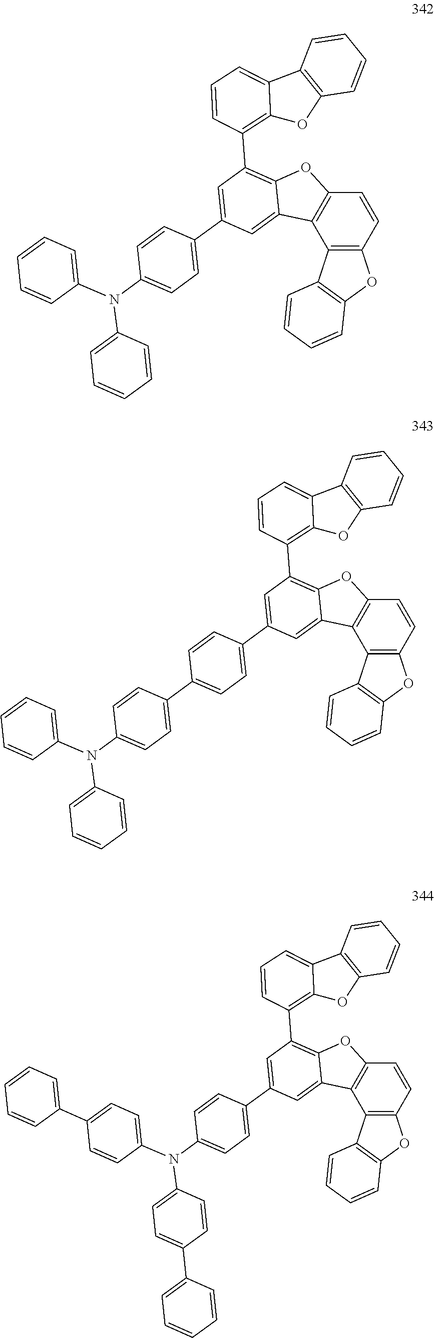 [SMILES: c1ccc(N(c2ccccc2)c2ccc(-c3cc(-c4cccc5c4oc4ccccc45)c4oc5ccc6oc7ccccc7c6c5c4c3)cc2)cc1 ; c1ccc(N(c2ccccc2)c2ccc(-c3ccc(-c4cc(-c5cccc6c5oc5ccccc56)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5cccc6c5oc5ccccc56)c5oc6ccc7oc8ccccc8c7c6c5c4)cc3)cc2)cc1]

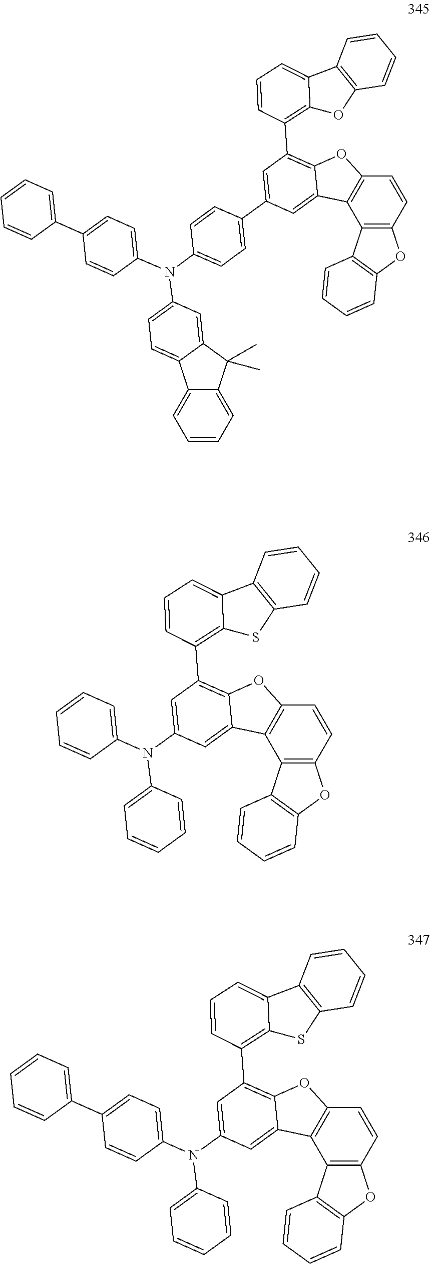 [SMILES: CC1(C)c2ccccc2-c2ccc(N(c3ccc(-c4ccccc4)cc3)c3ccc(-c4cc(-c5cccc6c5oc5ccccc56)c5oc6ccc7oc8ccccc8c7c6c5c4)cc3)cc21 ; c1ccc(N(c2ccccc2)c2cc(-c3cccc4c3sc3ccccc34)c3oc4ccc5oc6ccccc6c5c4c3c2)cc1 ; c1ccc(-c2ccc(N(c3ccccc3)c3cc(-c4cccc5c4sc4ccccc45)c4oc5ccc6oc7ccccc7c6c5c4c3)cc2)cc1]

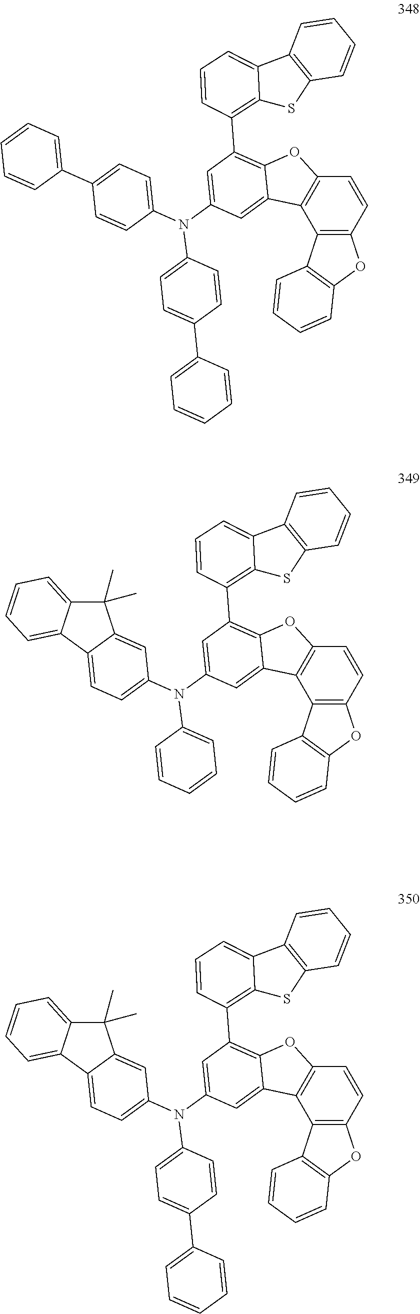 [SMILES: c1ccc(-c2ccc(N(c3ccc(-c4ccccc4)cc3)c3cc(-c4cccc5c4sc4ccccc45)c4oc5ccc6oc7ccccc7c6c5c4c3)cc2)cc1 ; CC1(C)c2ccccc2-c2ccc(N(c3ccccc3)c3cc(-c4cccc5c4sc4ccccc45)c4oc5ccc6oc7ccccc7c6c5c4c3)cc21 ; CC1(C)c2ccccc2-c2ccc(N(c3ccc(-c4ccccc4)cc3)c3cc(-c4cccc5c4sc4ccccc45)c4oc5ccc6oc7ccccc7c6c5c4c3)cc21]

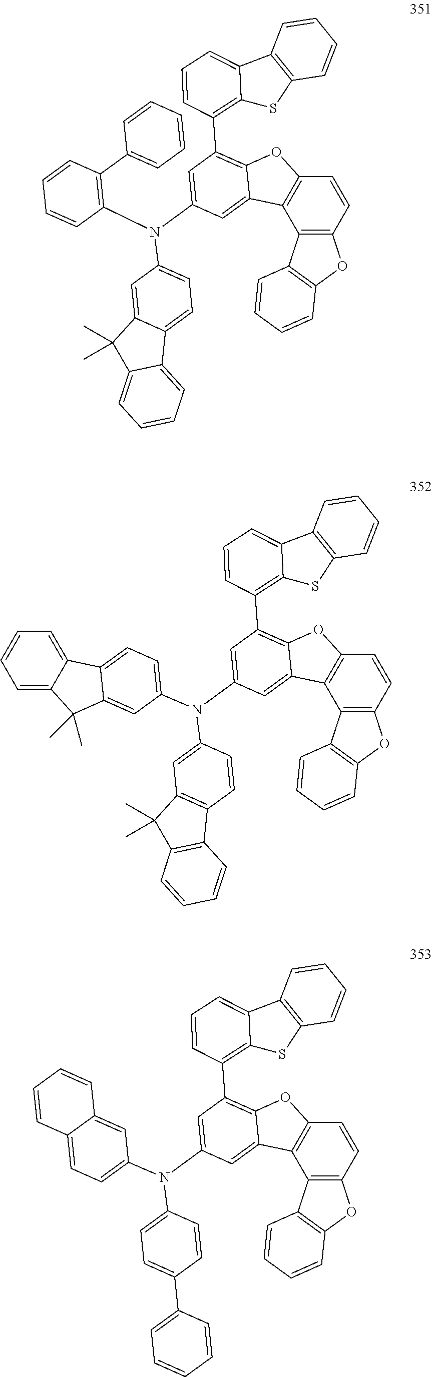 [SMILES: CC1(C)c2ccccc2-c2ccc(N(c3cc(-c4cccc5c4sc4ccccc45)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4cccc5c4sc4ccccc45)c4oc5ccc6oc7ccccc7c6c5c4c3)cc21 ; c1ccc(-c2ccc(N(c3ccc4ccccc4c3)c3cc(-c4cccc5c4sc4ccccc45)c4oc5ccc6oc7ccccc7c6c5c4c3)cc2)cc1]

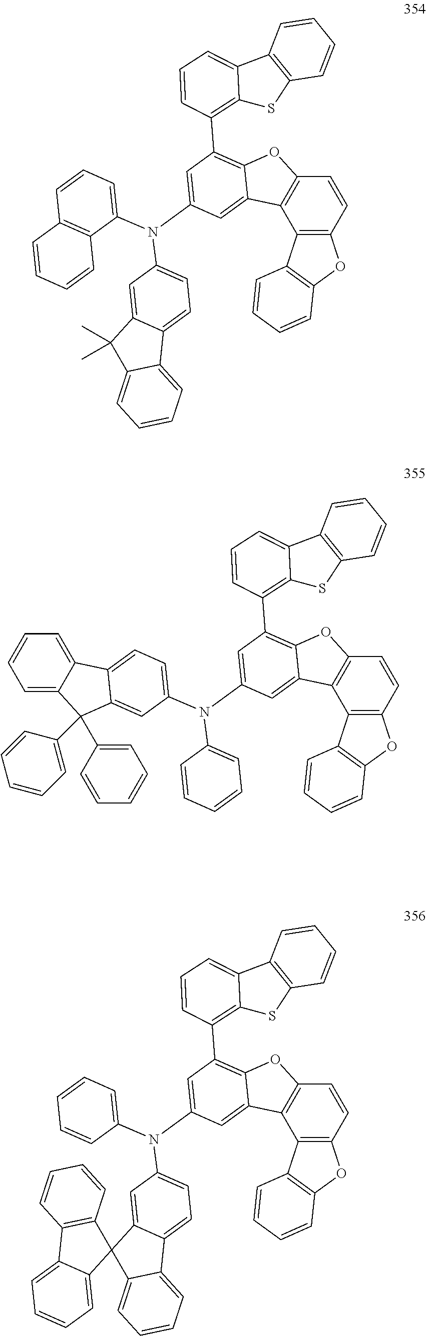 [SMILES: CC1(C)c2ccccc2-c2ccc(N(c3cc(-c4cccc5c4sc4ccccc45)c4oc5ccc6oc7ccccc7c6c5c4c3)c3cccc4ccccc34)cc21 ; c1ccc(N(c2ccc3c(c2)C(c2ccccc2)(c2ccccc2)c2ccccc2-3)c2cc(-c3cccc4c3sc3ccccc34)c3oc4ccc5oc6ccccc6c5c4c3c2)cc1 ; c1ccc(N(c2ccc3c(c2)C2(c4ccccc4-c4ccccc42)c2ccccc2-3)c2cc(-c3cccc4c3sc3ccccc34)c3oc4ccc5oc6ccccc6c5c4c3c2)cc1]

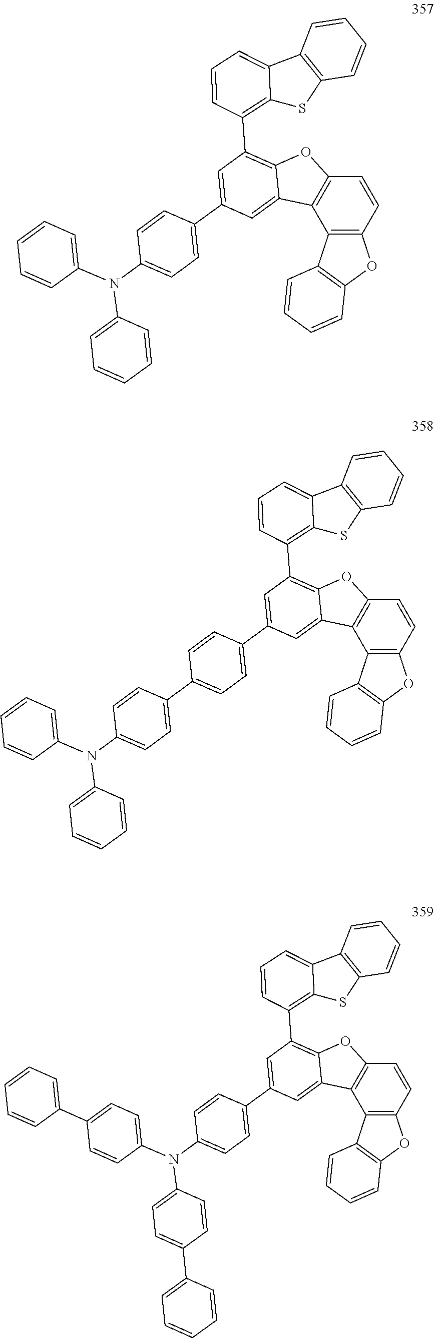 [SMILES: c1ccc(N(c2ccccc2)c2ccc(-c3cc(-c4cccc5c4sc4ccccc45)c4oc5ccc6oc7ccccc7c6c5c4c3)cc2)cc1 ; c1ccc(N(c2ccccc2)c2ccc(-c3ccc(-c4cc(-c5cccc6c5sc5ccccc56)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5cccc6c5sc5ccccc56)c5oc6ccc7oc8ccccc8c7c6c5c4)cc3)cc2)cc1]

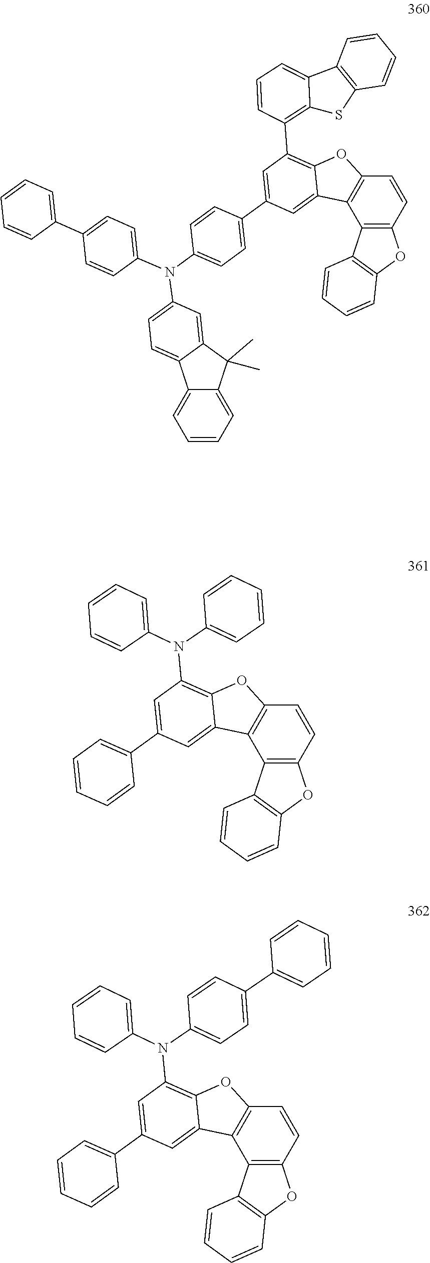 [SMILES: CC1(C)c2ccccc2-c2ccc(N(c3ccc(-c4ccccc4)cc3)c3ccc(-c4cc(-c5cccc6c5sc5ccccc56)c5oc6ccc7oc8ccccc8c7c6c5c4)cc3)cc21 ; c1ccc(-c2cc(N(c3ccccc3)c3ccccc3)c3oc4ccc5oc6ccccc6c5c4c3c2)cc1 ; c1ccc(-c2ccc(N(c3ccccc3)c3cc(-c4ccccc4)cc4c3oc3ccc5oc6ccccc6c5c34)cc2)cc1]

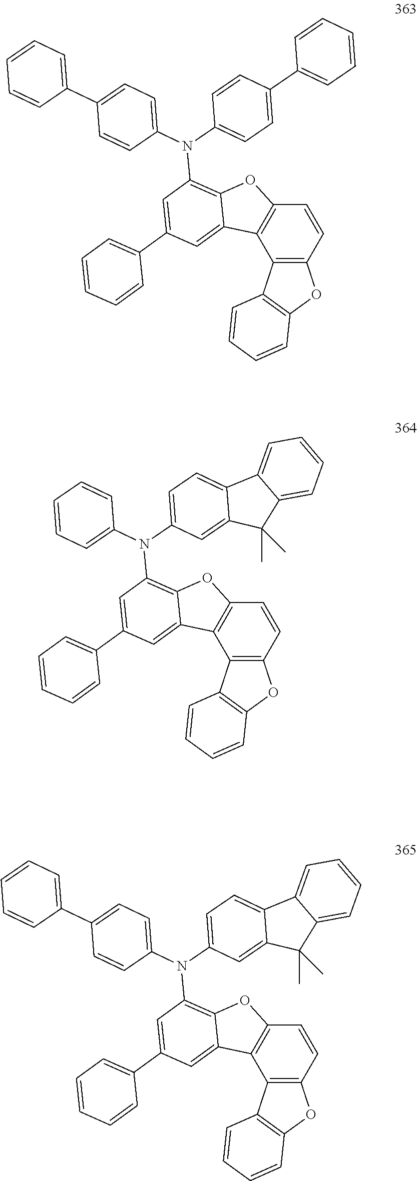 [SMILES: c1ccc(-c2ccc(N(c3ccc(-c4ccccc4)cc3)c3cc(-c4ccccc4)cc4c3oc3ccc5oc6ccccc6c5c34)cc2)cc1 ; CC1(C)c2ccccc2-c2ccc(N(c3ccccc3)c3cc(-c4ccccc4)cc4c3oc3ccc5oc6ccccc6c5c34)cc21 ; CC1(C)c2ccccc2-c2ccc(N(c3ccc(-c4ccccc4)cc3)c3cc(-c4ccccc4)cc4c3oc3ccc5oc6ccccc6c5c34)cc21]

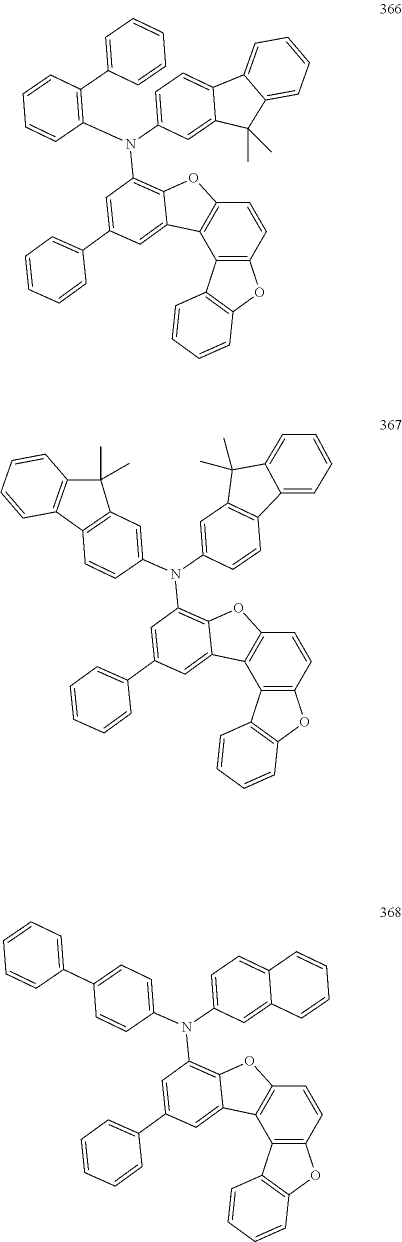 [SMILES: CC1(C)c2ccccc2-c2ccc(N(c3ccccc3-c3ccccc3)c3cc(-c4ccccc4)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4ccccc4)cc4c3oc3ccc5oc6ccccc6c5c34)cc21 ; c1ccc(-c2ccc(N(c3ccc4ccccc4c3)c3cc(-c4ccccc4)cc4c3oc3ccc5oc6ccccc6c5c34)cc2)cc1]

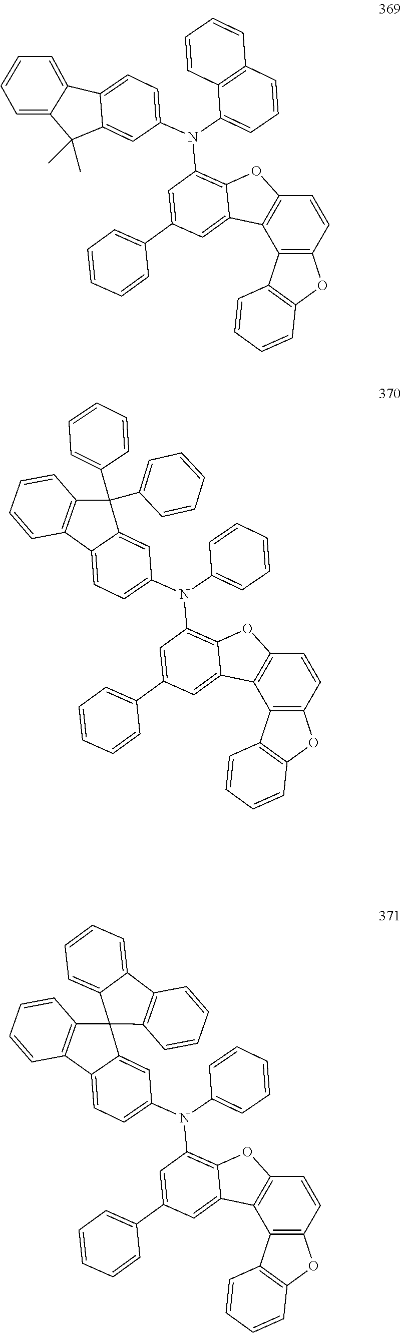 [SMILES: CC1(C)c2ccccc2-c2ccc(N(c3cccc4ccccc34)c3cc(-c4ccccc4)cc4c3oc3ccc5oc6ccccc6c5c34)cc21 ; c1ccc(-c2cc(N(c3ccccc3)c3ccc4c(c3)C(c3ccccc3)(c3ccccc3)c3ccccc3-4)c3oc4ccc5oc6ccccc6c5c4c3c2)cc1 ; c1ccc(-c2cc(N(c3ccccc3)c3ccc4c(c3)C3(c5ccccc5-c5ccccc53)c3ccccc3-4)c3oc4ccc5oc6ccccc6c5c4c3c2)cc1]

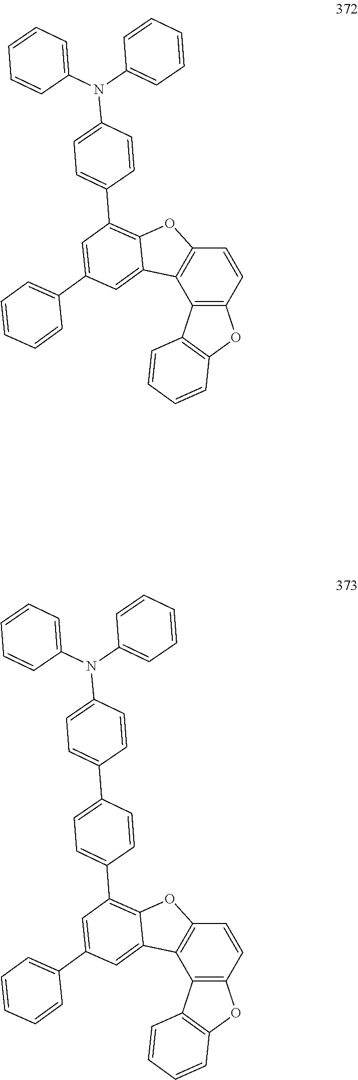 [SMILES: c1ccc(-c2cc(-c3ccc(N(c4ccccc4)c4ccccc4)cc3)c3oc4ccc5oc6ccccc6c5c4c3c2)cc1 ; c1ccc(-c2cc(-c3ccc(-c4ccc(N(c5ccccc5)c5ccccc5)cc4)cc3)c3oc4ccc5oc6ccccc6c5c4c3c2)cc1]

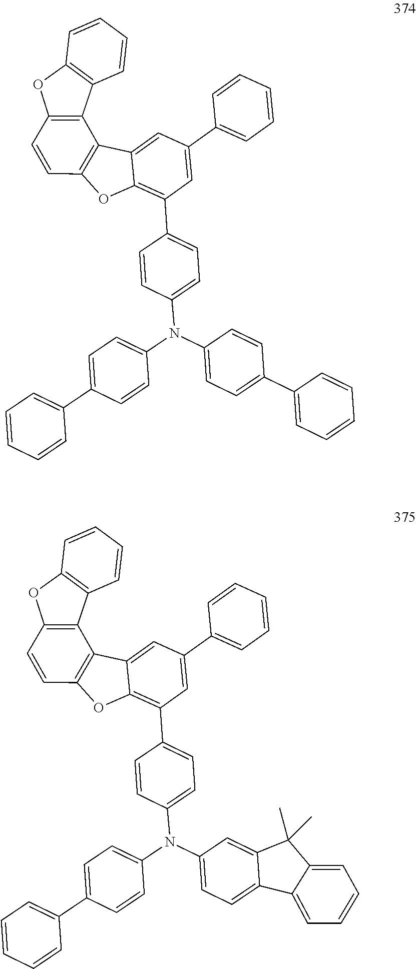 [SMILES: c1ccc(-c2ccc(N(c3ccc(-c4ccccc4)cc3)c3ccc(-c4cc(-c5ccccc5)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5ccccc5)cc5c4oc4ccc6oc7ccccc7c6c45)cc3)cc21]

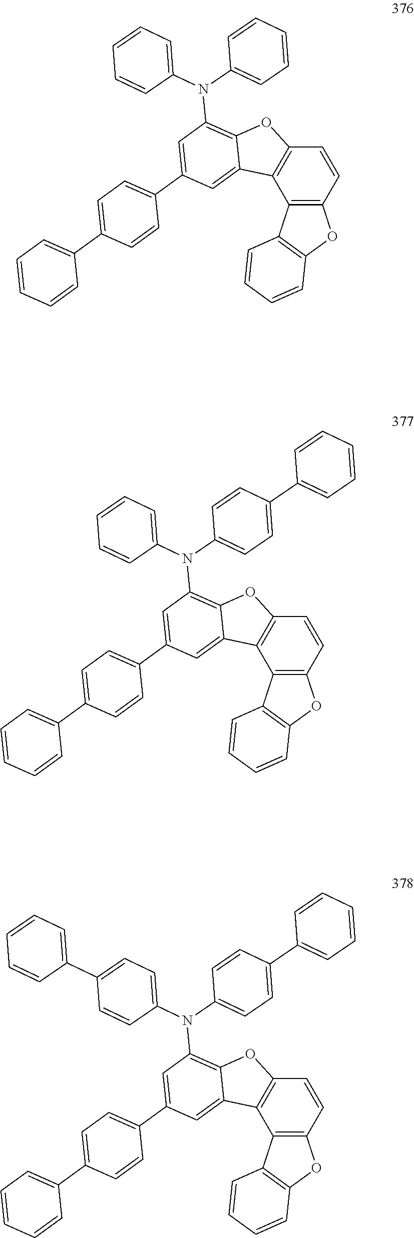 [SMILES: c1ccc(-c2ccc(-c3cc(N(c4ccccc4)c4ccccc4)c4oc5ccc6oc7ccccc7c6c5c4c3)cc2)cc1 ; c1ccc(-c2ccc(-c3cc(N(c4ccccc4)c4ccc(-c5ccccc5)cc4)c4oc5ccc6oc7ccccc7c6c5c4c3)cc2)cc1 ; c1ccc(-c2ccc(-c3cc(N(c4ccc(-c5ccccc5)cc4)c4ccc(-c5ccccc5)cc4)c4oc5ccc6oc7ccccc7c6c5c4c3)cc2)cc1]

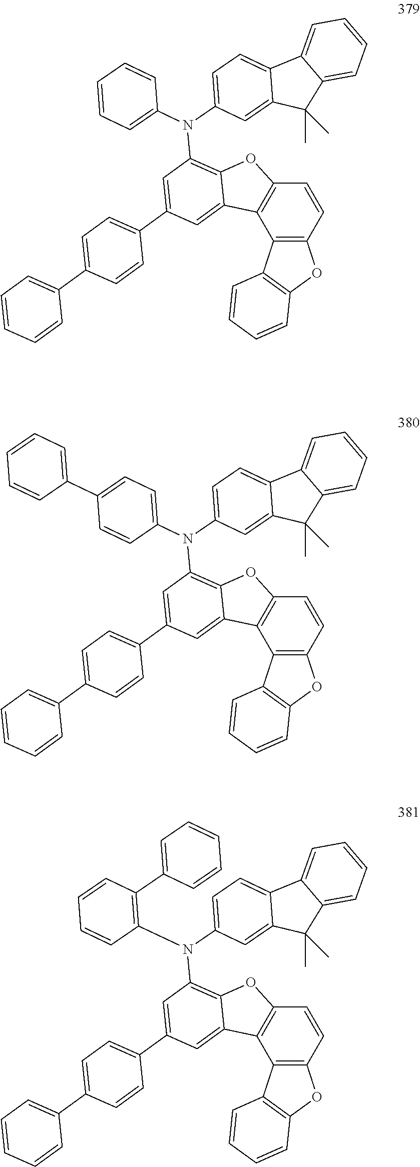 [SMILES: CC1(C)c2ccccc2-c2ccc(N(c3ccccc3)c3cc(-c4ccc(-c5ccccc5)cc4)cc4c3oc3ccc5oc6ccccc6c5c34)cc21 ; CC1(C)c2ccccc2-c2ccc(N(c3ccc(-c4ccccc4)cc3)c3cc(-c4ccc(-c5ccccc5)cc4)cc4c3oc3ccc5oc6ccccc6c5c34)cc21 ; CC1(C)c2ccccc2-c2ccc(N(c3ccccc3-c3ccccc3)c3cc(-c4ccc(-c5ccccc5)cc4)cc4c3oc3ccc5oc6ccccc6c5c34)cc21]

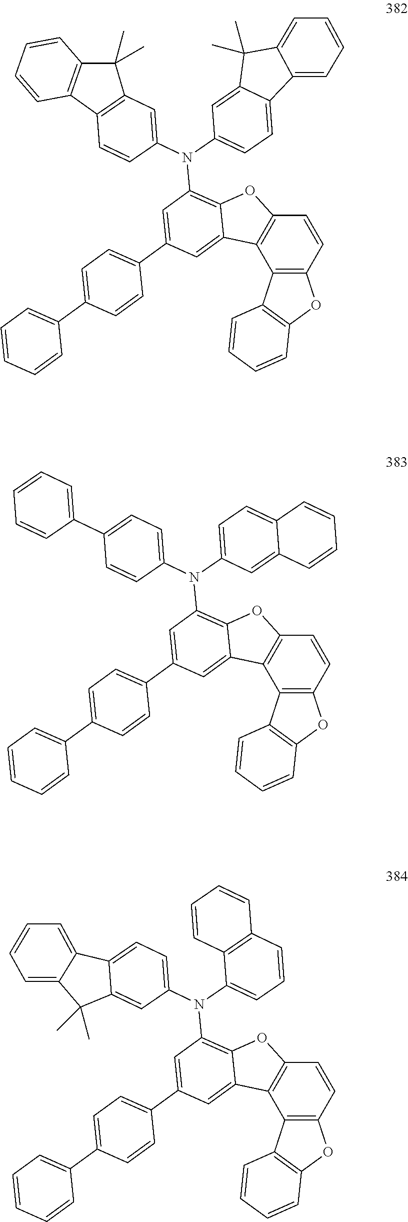 [SMILES: CC1(C)c2ccccc2-c2ccc(N(c3ccc4c(c3)C(C)(C)c3ccccc3-4)c3cc(-c4ccc(-c5ccccc5)cc4)cc4c3oc3ccc5oc6ccccc6c5c34)cc21 ; c1ccc(-c2ccc(-c3cc(N(c4ccc(-c5ccccc5)cc4)c4ccc5ccccc5c4)c4oc5ccc6oc7ccccc7c6c5c4c3)cc2)cc1 ; CC1(C)c2ccccc2-c2ccc(N(c3cccc4ccccc34)c3cc(-c4ccc(-c5ccccc5)cc4)cc4c3oc3ccc5oc6ccccc6c5c34)cc21]

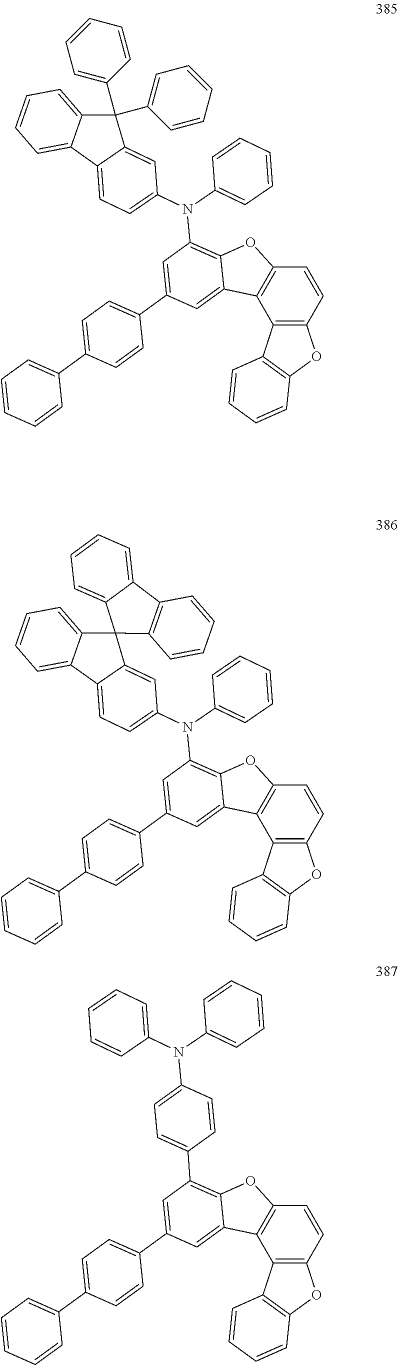 [SMILES: c1ccc(-c2ccc(-c3cc(N(c4ccccc4)c4ccc5c(c4)C(c4ccccc4)(c4ccccc4)c4ccccc4-5)c4oc5ccc6oc7ccccc7c6c5c4c3)cc2)cc1 ; c1ccc(-c2ccc(-c3cc(N(c4ccccc4)c4ccc5c(c4)C4(c6ccccc6-c6ccccc64)c4ccccc4-5)c4oc5ccc6oc7ccccc7c6c5c4c3)cc2)cc1 ; c1ccc(-c2ccc(-c3cc(-c4ccc(N(c5ccccc5)c5ccccc5)cc4)c4oc5ccc6oc7ccccc7c6c5c4c3)cc2)cc1]

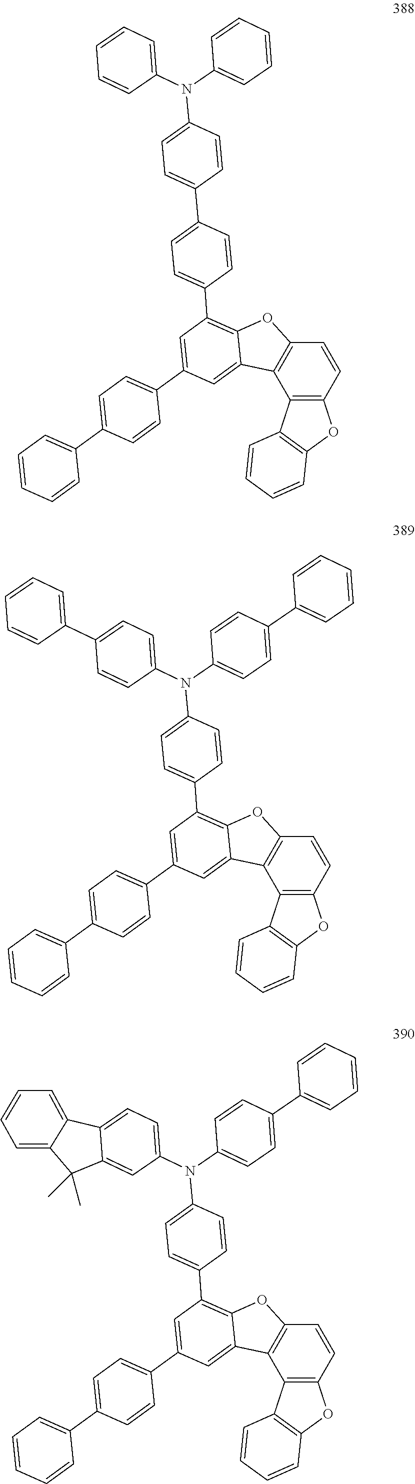 [SMILES: c1ccc(-c2ccc(-c3cc(-c4ccc(-c5ccc(N(c6ccccc6)c6ccccc6)cc5)cc4)c4oc5ccc6oc7ccccc7c6c5c4c3)cc2)cc1 ; c1ccc(-c2ccc(-c3cc(-c4ccc(N(c5ccc(-c6ccccc6)cc5)c5ccc(-c6ccccc6)cc5)cc4)c4oc5ccc6oc7ccccc7c6c5c4c3)cc2)cc1 ; CC1(C)c2ccccc2-c2ccc(N(c3ccc(-c4ccccc4)cc3)c3ccc(-c4cc(-c5ccc(-c6ccccc6)cc5)cc5c4oc4ccc6oc7ccccc7c6c45)cc3)cc21]

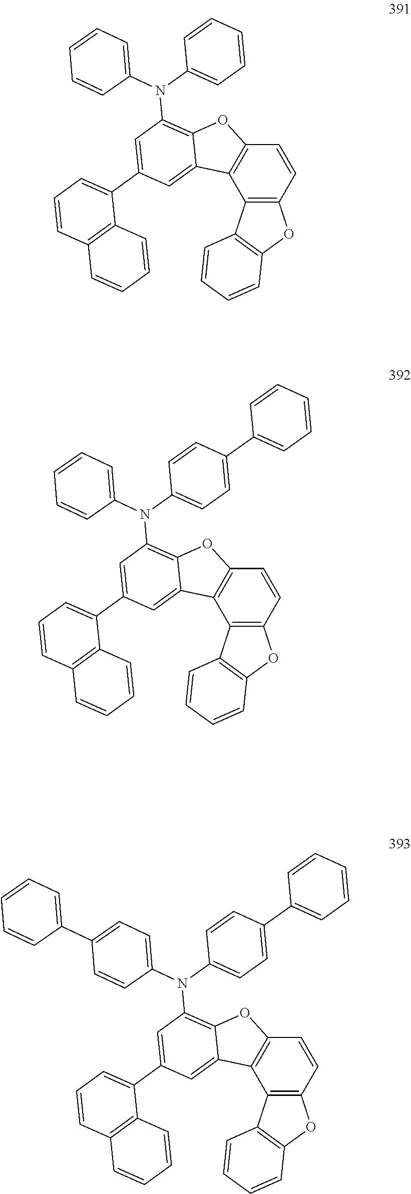 [SMILES: c1ccc(N(c2ccccc2)c2cc(-c3cccc4ccccc34)cc3c2oc2ccc4oc5ccccc5c4c23)cc1 ; c1ccc(-c2ccc(N(c3ccccc3)c3cc(-c4cccc5ccccc45)cc4c3oc3ccc5oc6ccccc6c5c34)cc2)cc1 ; c1ccc(-c2ccc(N(c3ccc(-c4ccccc4)cc3)c3cc(-c4cccc5ccccc45)cc4c3oc3ccc5oc6ccccc6c5c34)cc2)cc1]

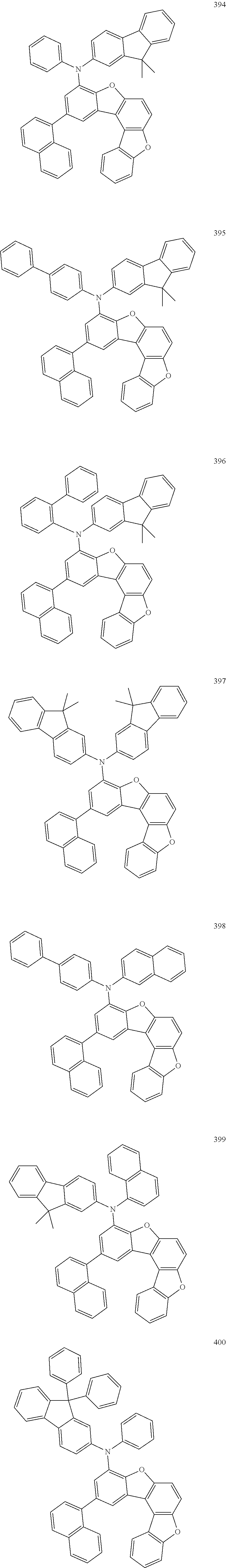

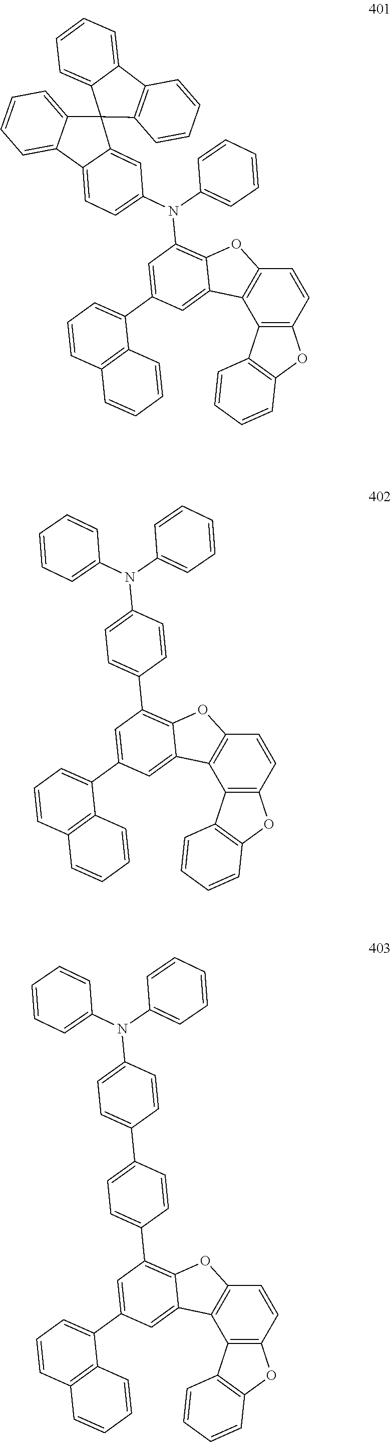 [SMILES: c1ccc(N(c2ccc3c(c2)C2(c4ccccc4-c4ccccc42)c2ccccc2-3)c2cc(-c3cccc4ccccc34)cc3c2oc2ccc4oc5ccccc5c4c23)cc1 ; c1ccc(N(c2ccccc2)c2ccc(-c3cc(-c4cccc5ccccc45)cc4c3oc3ccc5oc6ccccc6c5c34)cc2)cc1 ; c1ccc(N(c2ccccc2)c2ccc(-c3ccc(-c4cc(-c5cccc6ccccc56)cc5c4oc4ccc6oc7ccccc7c6c45)cc3)cc2)cc1]

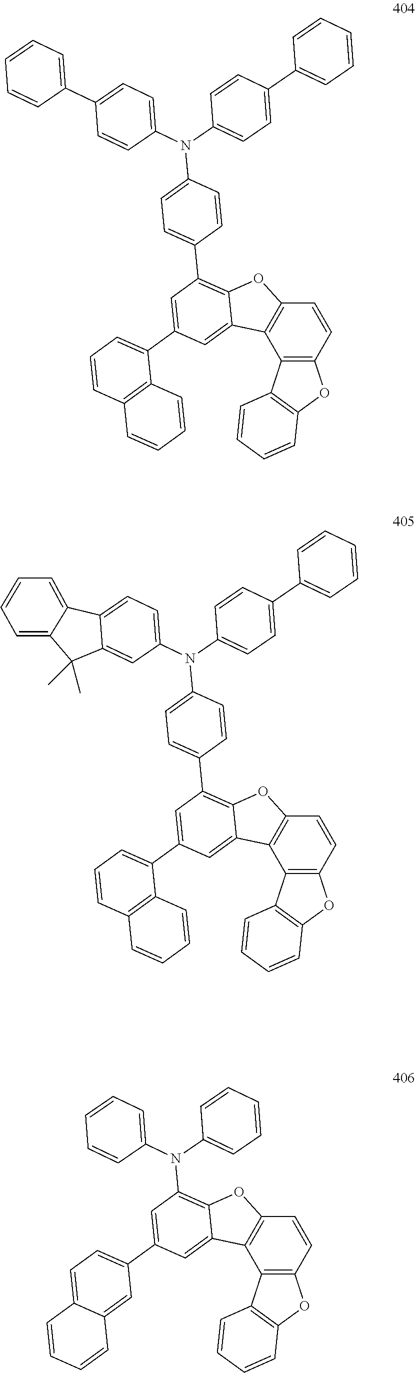 [SMILES: c1ccc(-c2ccc(N(c3ccc(-c4ccccc4)cc3)c3ccc(-c4cc(-c5cccc6ccccc56)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5cccc6ccccc56)cc5c4oc4ccc6oc7ccccc7c6c45)cc3)cc21 ; c1ccc(N(c2ccccc2)c2cc(-c3ccc4ccccc4c3)cc3c2oc2ccc4oc5ccccc5c4c23)cc1]

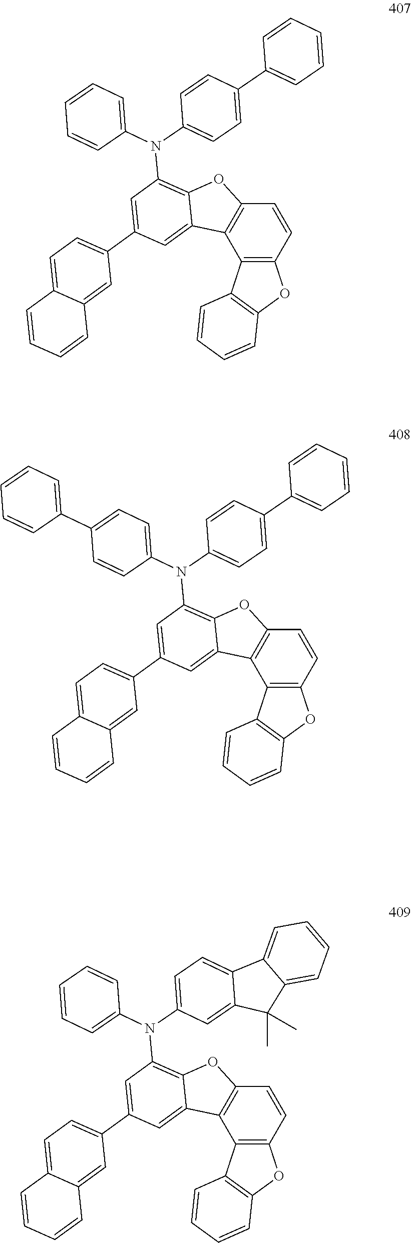 [SMILES: c1ccc(-c2ccc(N(c3ccccc3)c3cc(-c4ccc5ccccc5c4)cc4c3oc3ccc5oc6ccccc6c5c34)cc2)cc1 ; c1ccc(-c2ccc(N(c3ccc(-c4ccccc4)cc3)c3cc(-c4ccc5ccccc5c4)cc4c3oc3ccc5oc6ccccc6c5c34)cc2)cc1 ; CC1(C)c2ccccc2-c2ccc(N(c3ccccc3)c3cc(-c4ccc5ccccc5c4)cc4c3oc3ccc5oc6ccccc6c5c34)cc21]

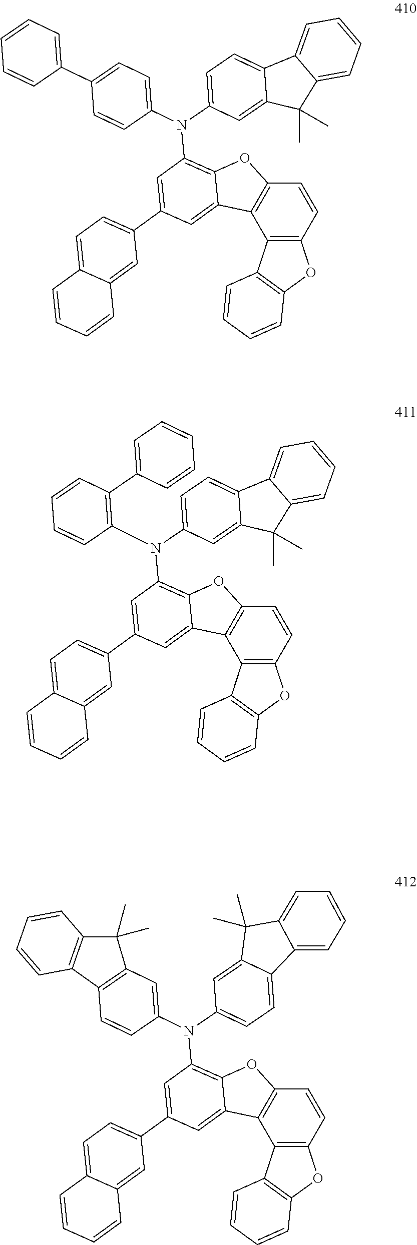 [SMILES: CC1(C)c2ccccc2-c2ccc(N(c3ccc(-c4ccccc4)cc3)c3cc(-c4ccc5ccccc5c4)cc4c3oc3ccc5oc6ccccc6c5c34)cc21 ; CC1(C)c2ccccc2-c2ccc(N(c3ccccc3-c3ccccc3)c3cc(-c4ccc5ccccc5c4)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4ccc5ccccc5c4)cc4c3oc3ccc5oc6ccccc6c5c34)cc21]

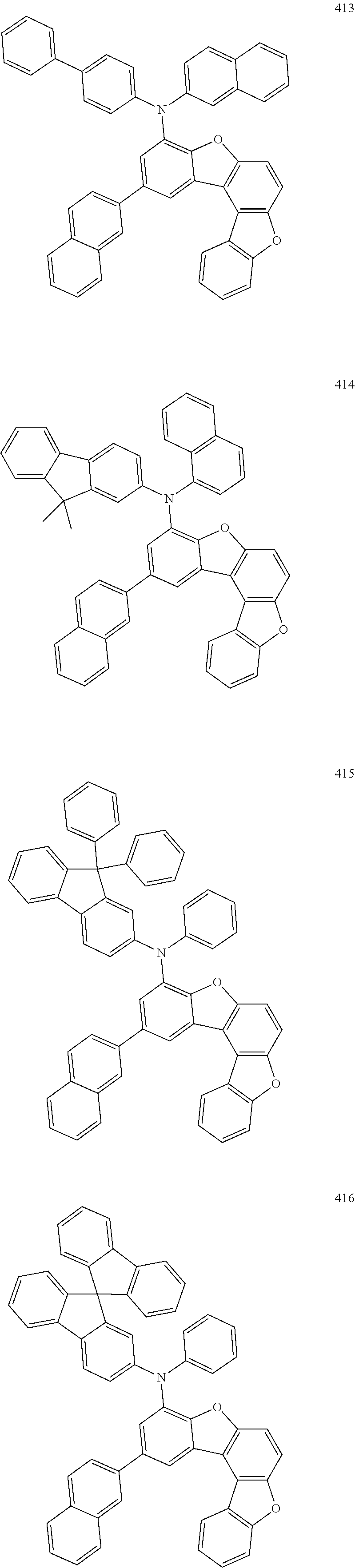 [SMILES: c1ccc(-c2ccc(N(c3ccc4ccccc4c3)c3cc(-c4ccc5ccccc5c4)cc4c3oc3ccc5oc6ccccc6c5c34)cc2)cc1 ; CC1(C)c2ccccc2-c2ccc(N(c3cccc4ccccc34)c3cc(-c4ccc5ccccc5c4)cc4c3oc3ccc5oc6ccccc6c5c34)cc21 ; c1ccc(N(c2ccc3c(c2)C(c2ccccc2)(c2ccccc2)c2ccccc2-3)c2cc(-c3ccc4ccccc4c3)cc3c2oc2ccc4oc5ccccc5c4c23)cc1 ; c1ccc(N(c2ccc3c(c2)C2(c4ccccc4-c4ccccc42)c2ccccc2-3)c2cc(-c3ccc4ccccc4c3)cc3c2oc2ccc4oc5ccccc5c4c23)cc1]

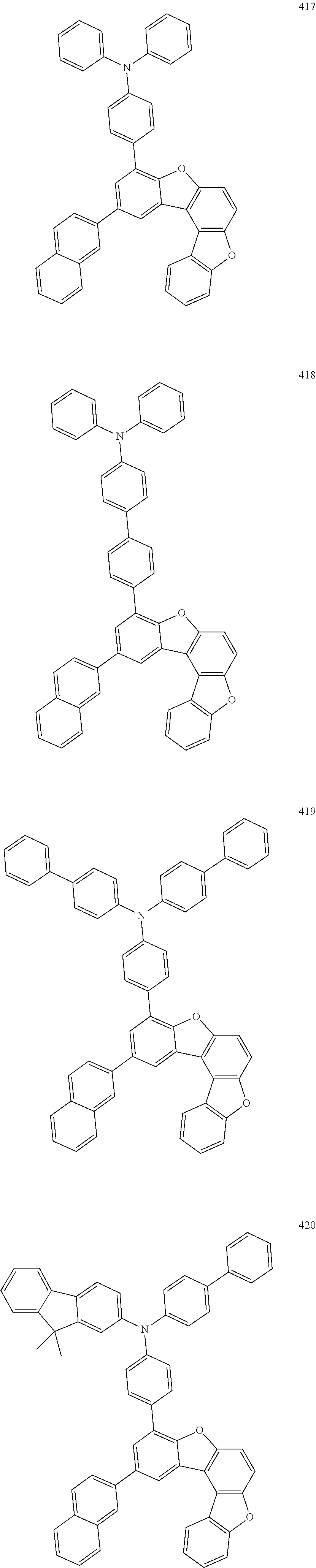 [SMILES: c1ccc(N(c2ccccc2)c2ccc(-c3cc(-c4ccc5ccccc5c4)cc4c3oc3ccc5oc6ccccc6c5c34)cc2)cc1 ; c1ccc(N(c2ccccc2)c2ccc(-c3ccc(-c4cc(-c5ccc6ccccc6c5)cc5c4oc4ccc6oc7ccccc7c6c45)cc3)cc2)cc1 ; c1ccc(-c2ccc(N(c3ccc(-c4ccccc4)cc3)c3ccc(-c4cc(-c5ccc6ccccc6c5)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5ccc6ccccc6c5)cc5c4oc4ccc6oc7ccccc7c6c45)cc3)cc21]

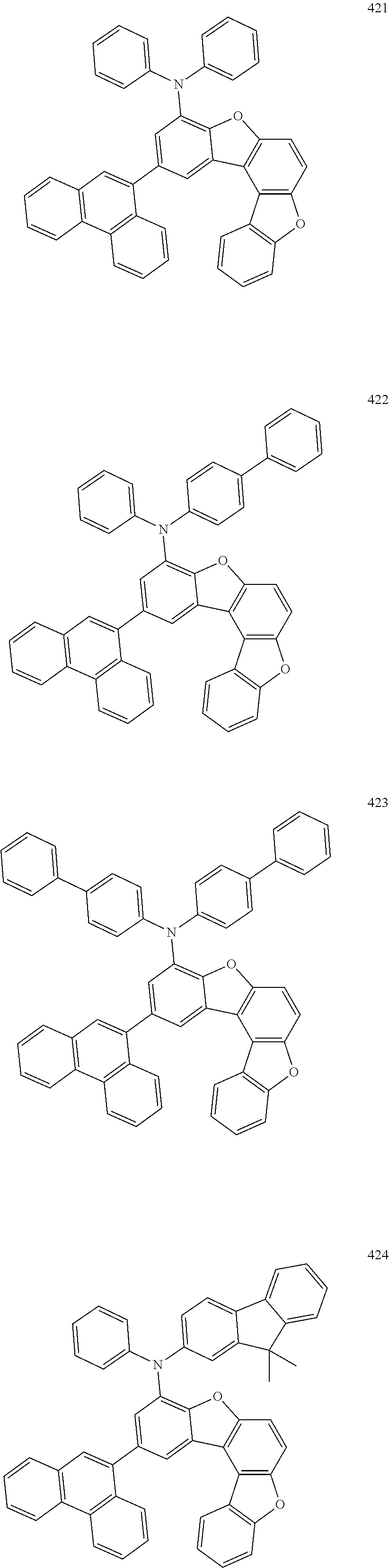 [SMILES: c1ccc(N(c2ccccc2)c2cc(-c3cc4ccccc4c4ccccc34)cc3c2oc2ccc4oc5ccccc5c4c23)cc1 ; c1ccc(-c2ccc(N(c3ccccc3)c3cc(-c4cc5ccccc5c5ccccc45)cc4c3oc3ccc5oc6ccccc6c5c34)cc2)cc1 ; c1ccc(-c2ccc(N(c3ccc(-c4ccccc4)cc3)c3cc(-c4cc5ccccc5c5ccccc45)cc4c3oc3ccc5oc6ccccc6c5c34)cc2)cc1 ; CC1(C)c2ccccc2-c2ccc(N(c3ccccc3)c3cc(-c4cc5ccccc5c5ccccc45)cc4c3oc3ccc5oc6ccccc6c5c34)cc21]

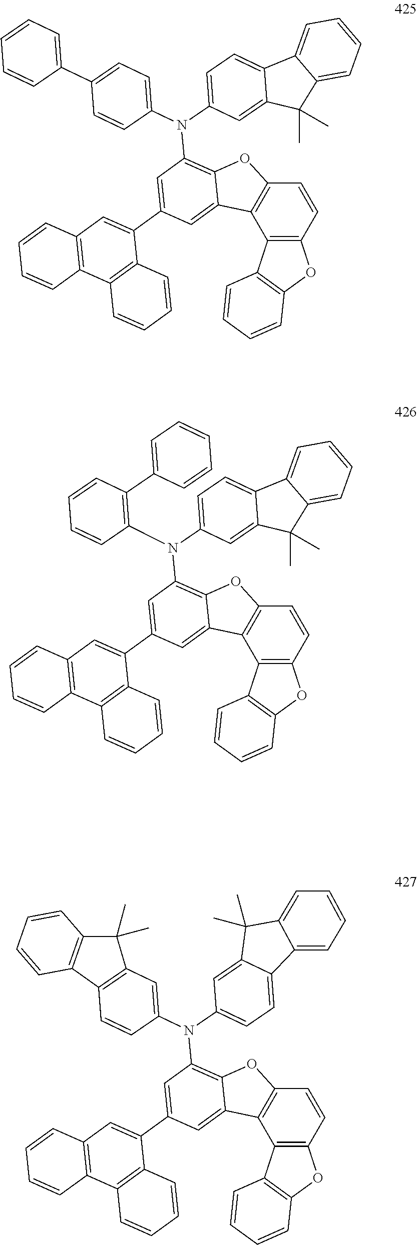 [SMILES: CC1(C)c2ccccc2-c2ccc(N(c3ccc(-c4ccccc4)cc3)c3cc(-c4cc5ccccc5c5ccccc45)cc4c3oc3ccc5oc6ccccc6c5c34)cc21 ; CC1(C)c2ccccc2-c2ccc(N(c3ccccc3-c3ccccc3)c3cc(-c4cc5ccccc5c5ccccc45)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4cc5ccccc5c5ccccc45)cc4c3oc3ccc5oc6ccccc6c5c34)cc21]

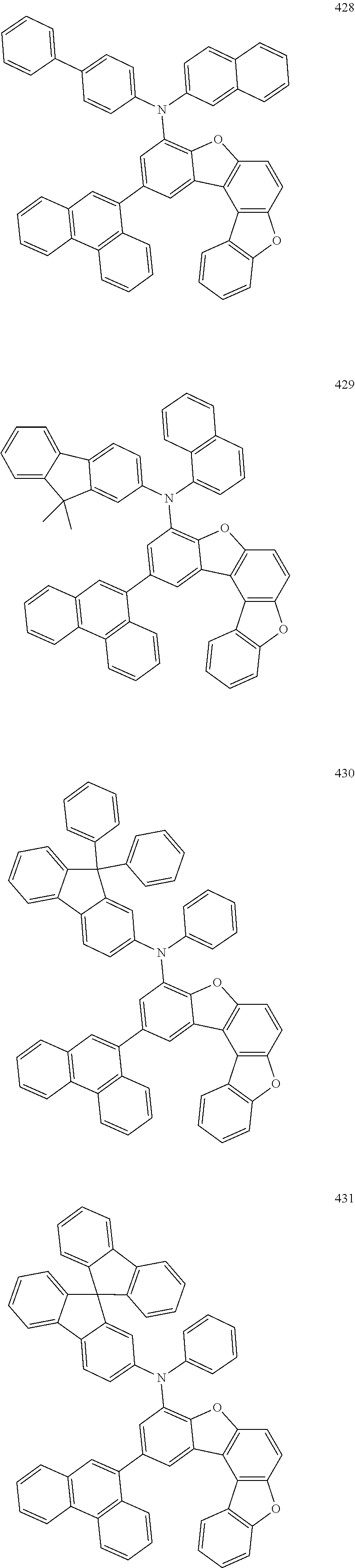 [SMILES: c1ccc(-c2ccc(N(c3ccc4ccccc4c3)c3cc(-c4cc5ccccc5c5ccccc45)cc4c3oc3ccc5oc6ccccc6c5c34)cc2)cc1 ; CC1(C)c2ccccc2-c2ccc(N(c3cccc4ccccc34)c3cc(-c4cc5ccccc5c5ccccc45)cc4c3oc3ccc5oc6ccccc6c5c34)cc21 ; c1ccc(N(c2ccc3c(c2)C(c2ccccc2)(c2ccccc2)c2ccccc2-3)c2cc(-c3cc4ccccc4c4ccccc34)cc3c2oc2ccc4oc5ccccc5c4c23)cc1 ; c1ccc(N(c2ccc3c(c2)C2(c4ccccc4-c4ccccc42)c2ccccc2-3)c2cc(-c3cc4ccccc4c4ccccc34)cc3c2oc2ccc4oc5ccccc5c4c23)cc1]

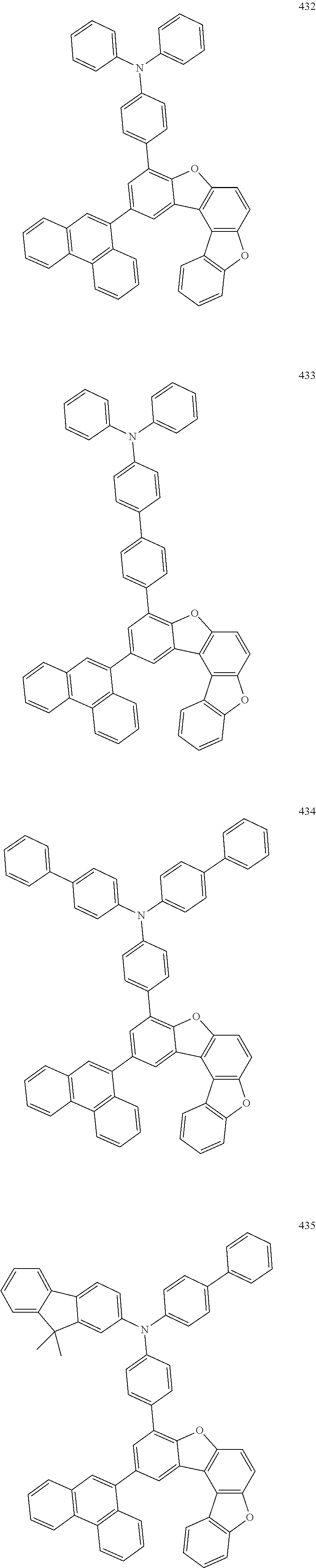 [SMILES: c1ccc(N(c2ccccc2)c2ccc(-c3cc(-c4cc5ccccc5c5ccccc45)cc4c3oc3ccc5oc6ccccc6c5c34)cc2)cc1 ; c1ccc(N(c2ccccc2)c2ccc(-c3ccc(-c4cc(-c5cc6ccccc6c6ccccc56)cc5c4oc4ccc6oc7ccccc7c6c45)cc3)cc2)cc1 ; c1ccc(-c2ccc(N(c3ccc(-c4ccccc4)cc3)c3ccc(-c4cc(-c5cc6ccccc6c6ccccc56)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5cc6ccccc6c6ccccc56)cc5c4oc4ccc6oc7ccccc7c6c45)cc3)cc21]

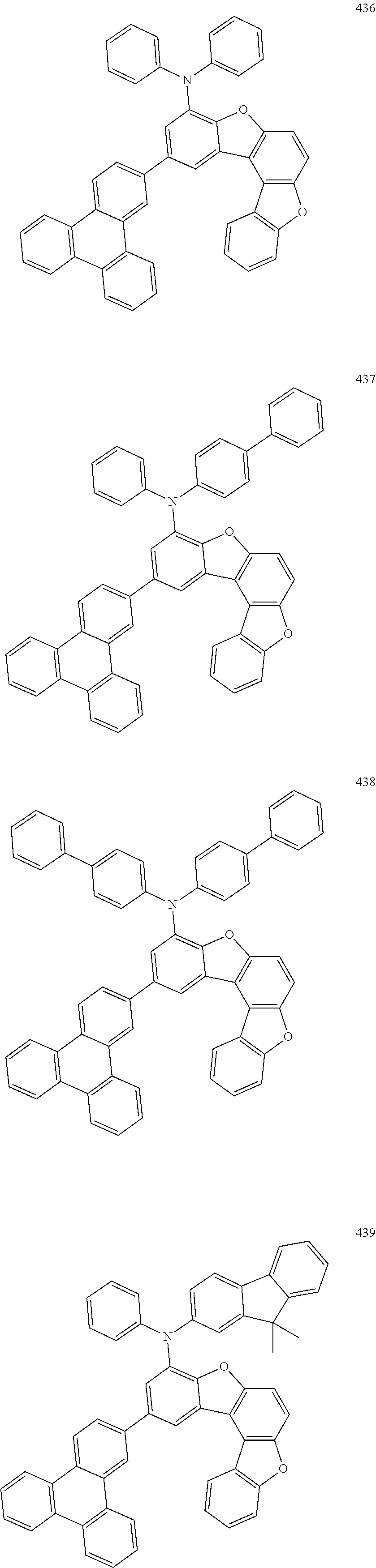 [SMILES: c1ccc(N(c2ccccc2)c2cc(-c3ccc4c5ccccc5c5ccccc5c4c3)cc3c2oc2ccc4oc5ccccc5c4c23)cc1 ; c1ccc(-c2ccc(N(c3ccccc3)c3cc(-c4ccc5c6ccccc6c6ccccc6c5c4)cc4c3oc3ccc5oc6ccccc6c5c34)cc2)cc1 ; c1ccc(-c2ccc(N(c3ccc(-c4ccccc4)cc3)c3cc(-c4ccc5c6ccccc6c6ccccc6c5c4)cc4c3oc3ccc5oc6ccccc6c5c34)cc2)cc1 ; CC1(C)c2ccccc2-c2ccc(N(c3ccccc3)c3cc(-c4ccc5c6ccccc6c6ccccc6c5c4)cc4c3oc3ccc5oc6ccccc6c5c34)cc21]

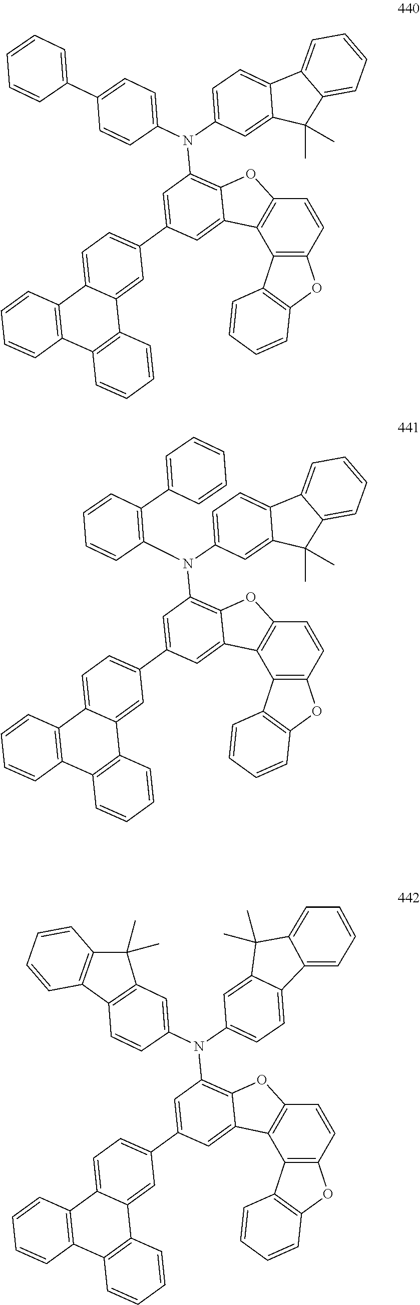 [SMILES: CC1(C)c2ccccc2-c2ccc(N(c3ccc(-c4ccccc4)cc3)c3cc(-c4ccc5c6ccccc6c6ccccc6c5c4)cc4c3oc3ccc5oc6ccccc6c5c34)cc21 ; CC1(C)c2ccccc2-c2ccc(N(c3ccccc3-c3ccccc3)c3cc(-c4ccc5c6ccccc6c6ccccc6c5c4)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4ccc5c6ccccc6c6ccccc6c5c4)cc4c3oc3ccc5oc6ccccc6c5c34)cc21]

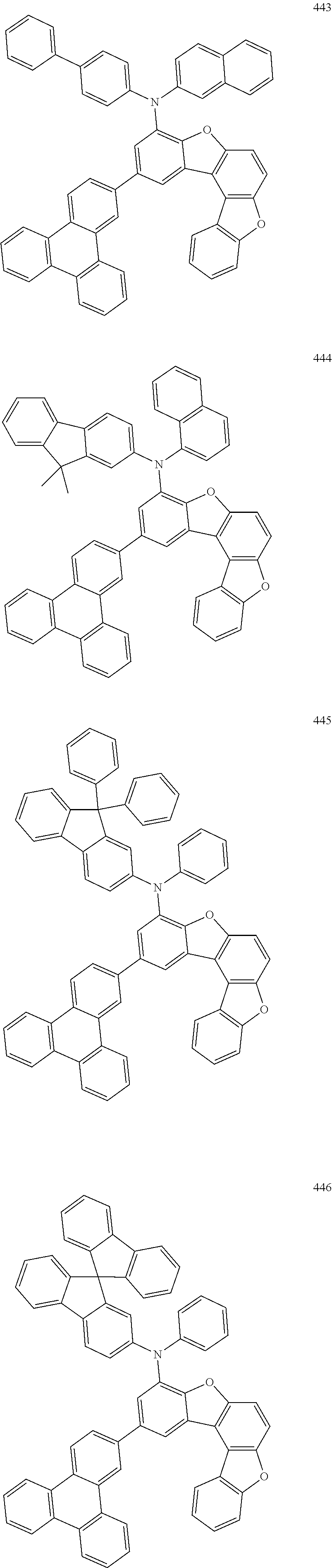 [SMILES: c1ccc(-c2ccc(N(c3ccc4ccccc4c3)c3cc(-c4ccc5c6ccccc6c6ccccc6c5c4)cc4c3oc3ccc5oc6ccccc6c5c34)cc2)cc1 ; CC1(C)c2ccccc2-c2ccc(N(c3cccc4ccccc34)c3cc(-c4ccc5c6ccccc6c6ccccc6c5c4)cc4c3oc3ccc5oc6ccccc6c5c34)cc21 ; c1ccc(N(c2ccc3c(c2)C(c2ccccc2)(c2ccccc2)c2ccccc2-3)c2cc(-c3ccc4c5ccccc5c5ccccc5c4c3)cc3c2oc2ccc4oc5ccccc5c4c23)cc1 ; c1ccc(N(c2ccc3c(c2)C2(c4ccccc4-c4ccccc42)c2ccccc2-3)c2cc(-c3ccc4c5ccccc5c5ccccc5c4c3)cc3c2oc2ccc4oc5ccccc5c4c23)cc1]

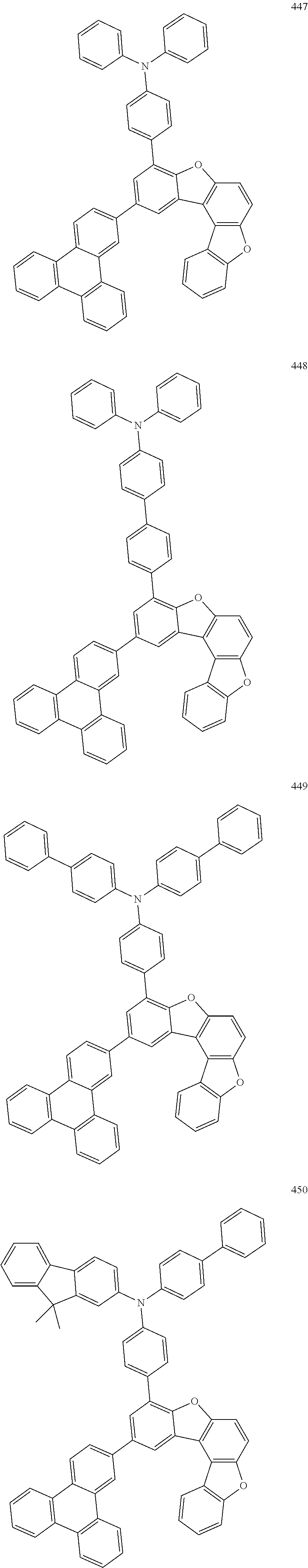 [SMILES: c1ccc(N(c2ccccc2)c2ccc(-c3cc(-c4ccc5c6ccccc6c6ccccc6c5c4)cc4c3oc3ccc5oc6ccccc6c5c34)cc2)cc1 ; c1ccc(N(c2ccccc2)c2ccc(-c3ccc(-c4cc(-c5ccc6c7ccccc7c7ccccc7c6c5)cc5c4oc4ccc6oc7ccccc7c6c45)cc3)cc2)cc1 ; c1ccc(-c2ccc(N(c3ccc(-c4ccccc4)cc3)c3ccc(-c4cc(-c5ccc6c7ccccc7c7ccccc7c6c5)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5ccc6c7ccccc7c7ccccc7c6c5)cc5c4oc4ccc6oc7ccccc7c6c45)cc3)cc21]

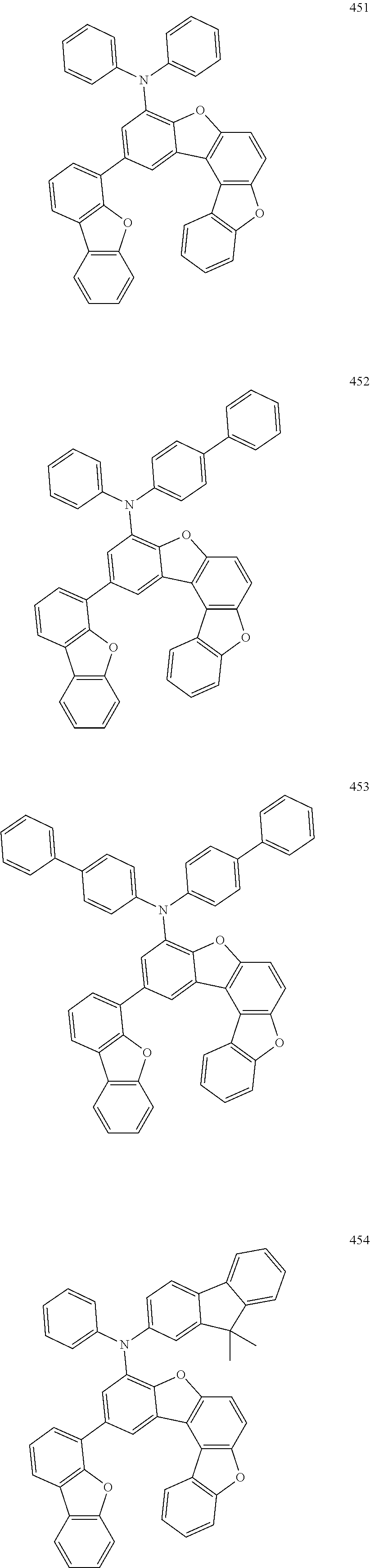 [SMILES: c1ccc(N(c2ccccc2)c2cc(-c3cccc4c3oc3ccccc34)cc3c2oc2ccc4oc5ccccc5c4c23)cc1 ; c1ccc(-c2ccc(N(c3ccccc3)c3cc(-c4cccc5c4oc4ccccc45)cc4c3oc3ccc5oc6ccccc6c5c34)cc2)cc1 ; c1ccc(-c2ccc(N(c3ccc(-c4ccccc4)cc3)c3cc(-c4cccc5c4oc4ccccc45)cc4c3oc3ccc5oc6ccccc6c5c34)cc2)cc1 ; CC1(C)c2ccccc2-c2ccc(N(c3ccccc3)c3cc(-c4cccc5c4oc4ccccc45)cc4c3oc3ccc5oc6ccccc6c5c34)cc21]

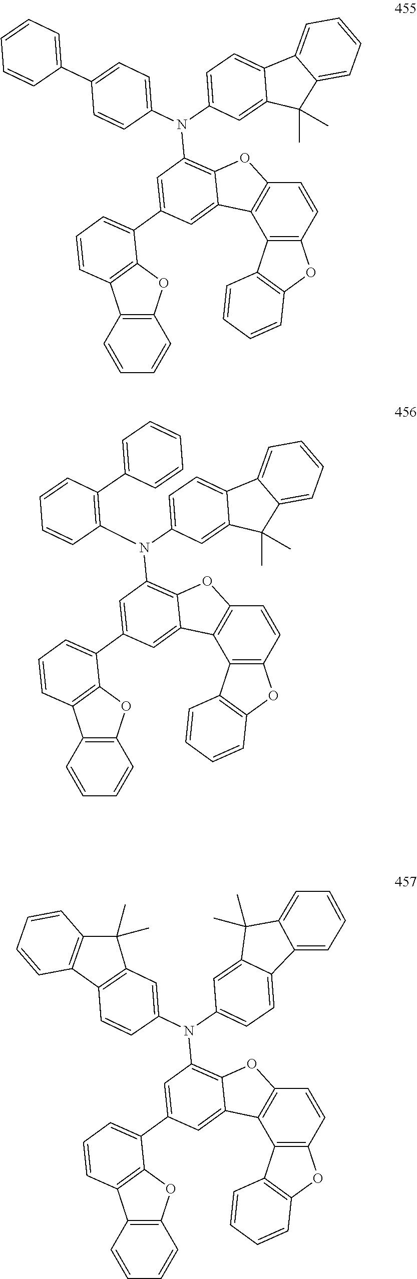 [SMILES: CC1(C)c2ccccc2-c2ccc(N(c3ccc(-c4ccccc4)cc3)c3cc(-c4cccc5c4oc4ccccc45)cc4c3oc3ccc5oc6ccccc6c5c34)cc21 ; CC1(C)c2ccccc2-c2ccc(N(c3ccccc3-c3ccccc3)c3cc(-c4cccc5c4oc4ccccc45)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4cccc5c4oc4ccccc45)cc4c3oc3ccc5oc6ccccc6c5c34)cc21]

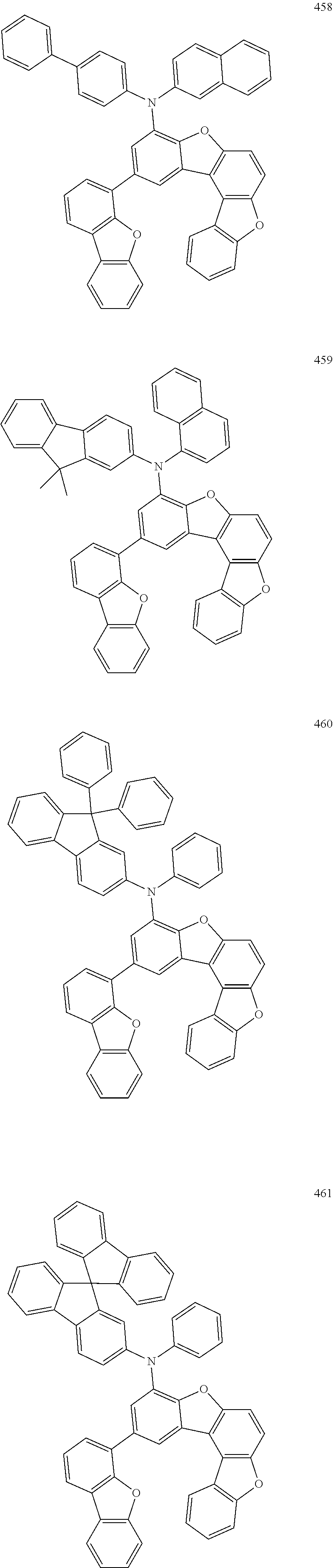 [SMILES: c1ccc(-c2ccc(N(c3ccc4ccccc4c3)c3cc(-c4cccc5c4oc4ccccc45)cc4c3oc3ccc5oc6ccccc6c5c34)cc2)cc1 ; CC1(C)c2ccccc2-c2ccc(N(c3cccc4ccccc34)c3cc(-c4cccc5c4oc4ccccc45)cc4c3oc3ccc5oc6ccccc6c5c34)cc21 ; c1ccc(N(c2ccc3c(c2)C(c2ccccc2)(c2ccccc2)c2ccccc2-3)c2cc(-c3cccc4c3oc3ccccc34)cc3c2oc2ccc4oc5ccccc5c4c23)cc1 ; c1ccc(N(c2ccc3c(c2)C2(c4ccccc4-c4ccccc42)c2ccccc2-3)c2cc(-c3cccc4c3oc3ccccc34)cc3c2oc2ccc4oc5ccccc5c4c23)cc1]

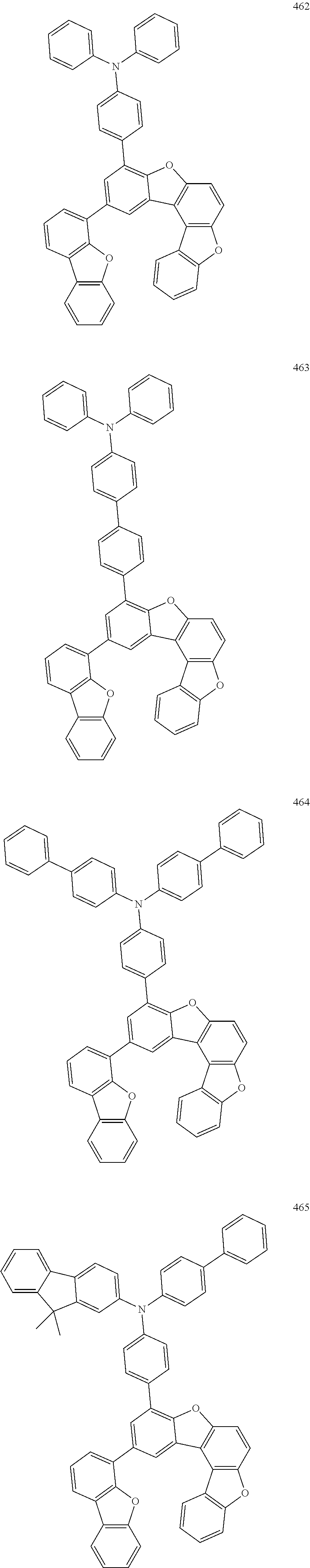 [SMILES: c1ccc(N(c2ccccc2)c2ccc(-c3cc(-c4cccc5c4oc4ccccc45)cc4c3oc3ccc5oc6ccccc6c5c34)cc2)cc1 ; c1ccc(N(c2ccccc2)c2ccc(-c3ccc(-c4cc(-c5cccc6c5oc5ccccc56)cc5c4oc4ccc6oc7ccccc7c6c45)cc3)cc2)cc1 ; c1ccc(-c2ccc(N(c3ccc(-c4ccccc4)cc3)c3ccc(-c4cc(-c5cccc6c5oc5ccccc56)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5cccc6c5oc5ccccc56)cc5c4oc4ccc6oc7ccccc7c6c45)cc3)cc21]

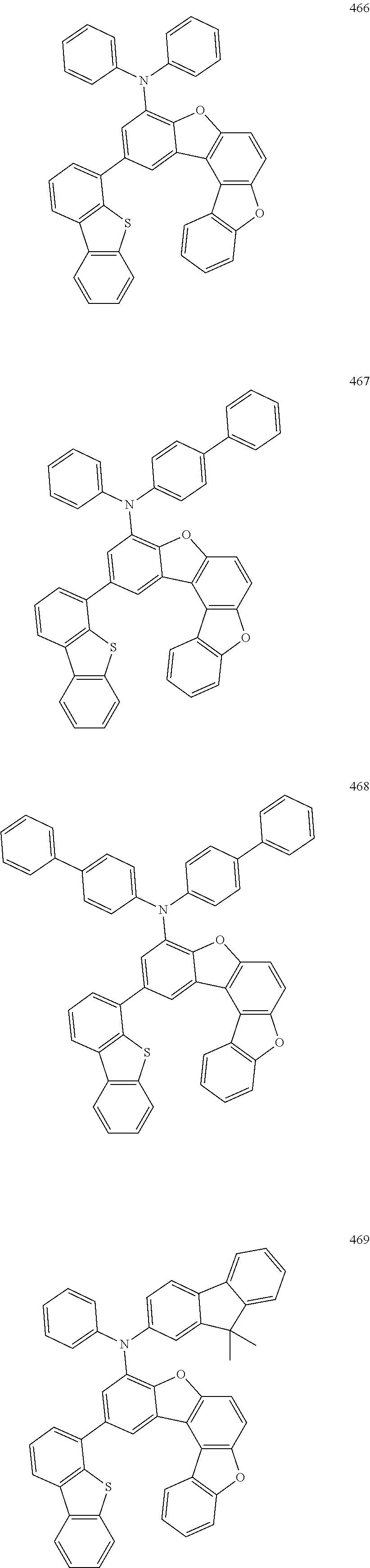 [SMILES: c1ccc(N(c2ccccc2)c2cc(-c3cccc4c3sc3ccccc34)cc3c2oc2ccc4oc5ccccc5c4c23)cc1 ; c1ccc(-c2ccc(N(c3ccccc3)c3cc(-c4cccc5c4sc4ccccc45)cc4c3oc3ccc5oc6ccccc6c5c34)cc2)cc1 ; c1ccc(-c2ccc(N(c3ccc(-c4ccccc4)cc3)c3cc(-c4cccc5c4sc4ccccc45)cc4c3oc3ccc5oc6ccccc6c5c34)cc2)cc1 ; CC1(C)c2ccccc2-c2ccc(N(c3ccccc3)c3cc(-c4cccc5c4sc4ccccc45)cc4c3oc3ccc5oc6ccccc6c5c34)cc21]

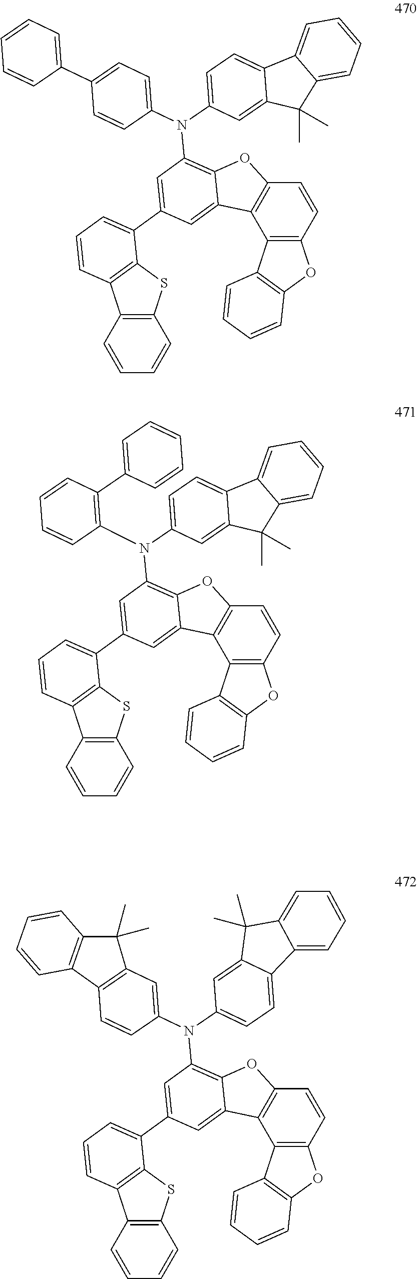 [SMILES: CC1(C)c2ccccc2-c2ccc(N(c3ccc(-c4ccccc4)cc3)c3cc(-c4cccc5c4sc4ccccc45)cc4c3oc3ccc5oc6ccccc6c5c34)cc21 ; CC1(C)c2ccccc2-c2ccc(N(c3ccccc3-c3ccccc3)c3cc(-c4cccc5c4sc4ccccc45)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4cccc5c4sc4ccccc45)cc4c3oc3ccc5oc6ccccc6c5c34)cc21]

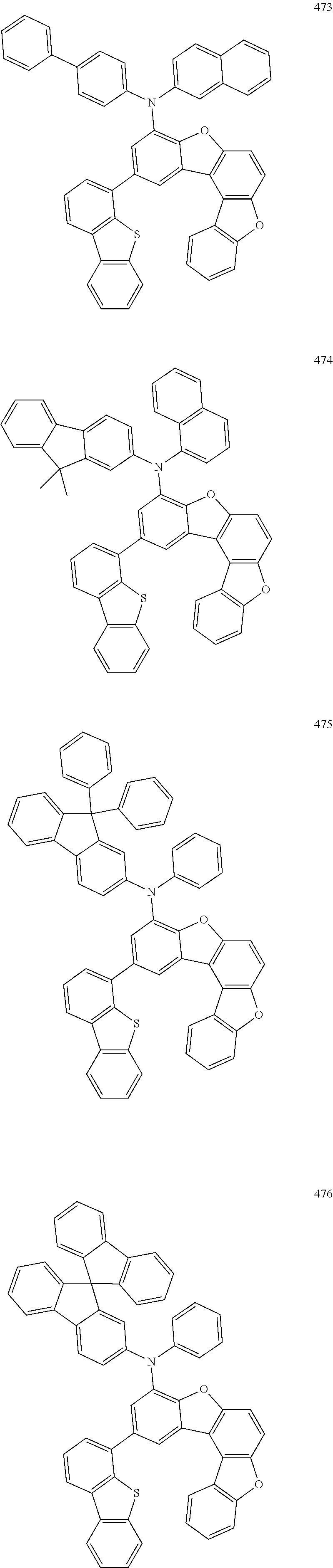 [SMILES: c1ccc(-c2ccc(N(c3ccc4ccccc4c3)c3cc(-c4cccc5c4sc4ccccc45)cc4c3oc3ccc5oc6ccccc6c5c34)cc2)cc1 ; CC1(C)c2ccccc2-c2ccc(N(c3cccc4ccccc34)c3cc(-c4cccc5c4sc4ccccc45)cc4c3oc3ccc5oc6ccccc6c5c34)cc21 ; c1ccc(N(c2ccc3c(c2)C(c2ccccc2)(c2ccccc2)c2ccccc2-3)c2cc(-c3cccc4c3sc3ccccc34)cc3c2oc2ccc4oc5ccccc5c4c23)cc1 ; c1ccc(N(c2ccc3c(c2)C2(c4ccccc4-c4ccccc42)c2ccccc2-3)c2cc(-c3cccc4c3sc3ccccc34)cc3c2oc2ccc4oc5ccccc5c4c23)cc1]

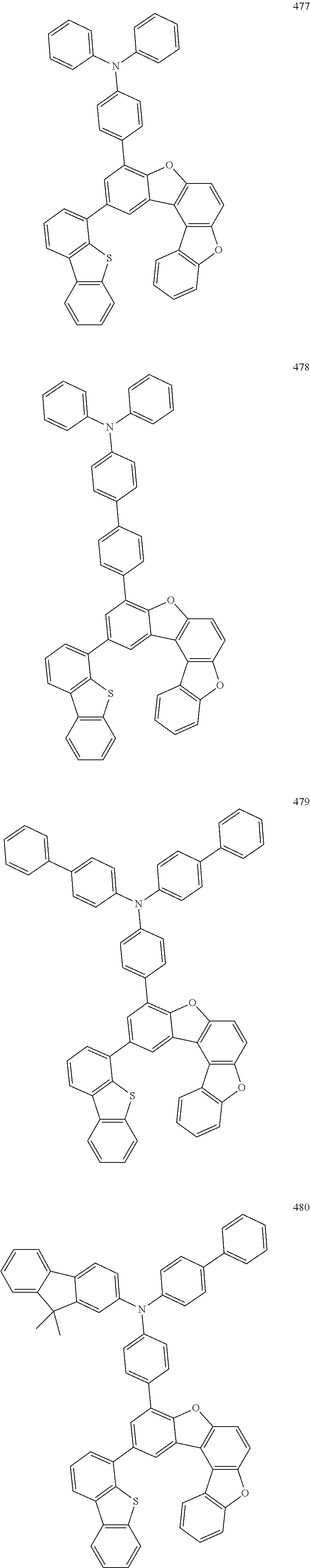 [SMILES: c1ccc(N(c2ccccc2)c2ccc(-c3cc(-c4cccc5c4sc4ccccc45)cc4c3oc3ccc5oc6ccccc6c5c34)cc2)cc1 ; c1ccc(N(c2ccccc2)c2ccc(-c3ccc(-c4cc(-c5cccc6c5sc5ccccc56)cc5c4oc4ccc6oc7ccccc7c6c45)cc3)cc2)cc1 ; c1ccc(-c2ccc(N(c3ccc(-c4ccccc4)cc3)c3ccc(-c4cc(-c5cccc6c5sc5ccccc56)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5cccc6c5sc5ccccc56)cc5c4oc4ccc6oc7ccccc7c6c45)cc3)cc21]

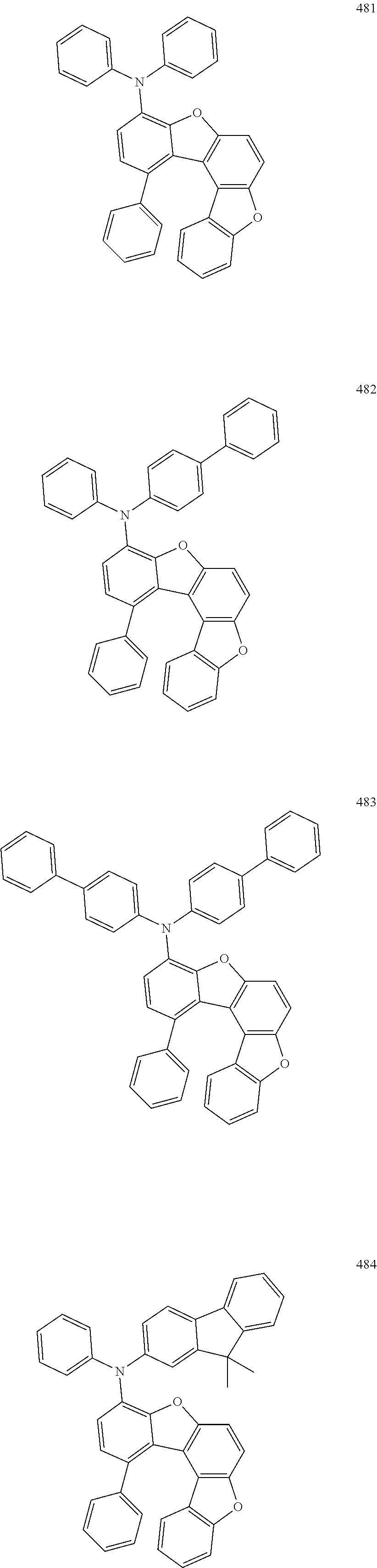 [SMILES: c1ccc(-c2ccc(N(c3ccccc3)c3ccccc3)c3oc4ccc5oc6ccccc6c5c4c23)cc1 ; c1ccc(-c2ccc(N(c3ccccc3)c3ccc(-c4ccccc4)c4c3oc3ccc5oc6ccccc6c5c34)cc2)cc1 ; c1ccc(-c2ccc(N(c3ccc(-c4ccccc4)cc3)c3ccc(-c4ccccc4)c4c3oc3ccc5oc6ccccc6c5c34)cc2)cc1 ; CC1(C)c2ccccc2-c2ccc(N(c3ccccc3)c3ccc(-c4ccccc4)c4c3oc3ccc5oc6ccccc6c5c34)cc21]

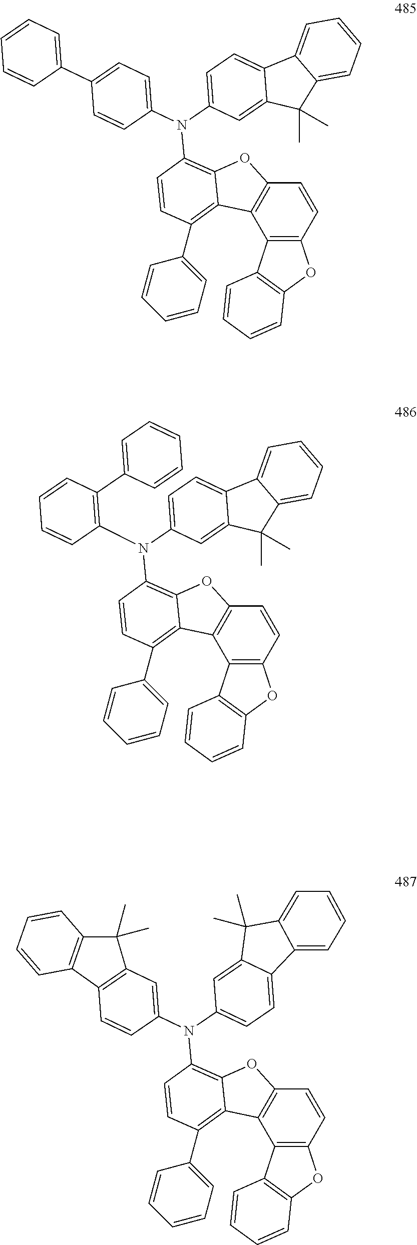 [SMILES: CC1(C)c2ccccc2-c2ccc(N(c3ccc(-c4ccccc4)cc3)c3ccc(-c4ccccc4)c4c3oc3ccc5oc6ccccc6c5c34)cc21 ; CC1(C)c2ccccc2-c2ccc(N(c3ccccc3-c3ccccc3)c3ccc(-c4ccccc4)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4ccccc4)c4c3oc3ccc5oc6ccccc6c5c34)cc21]

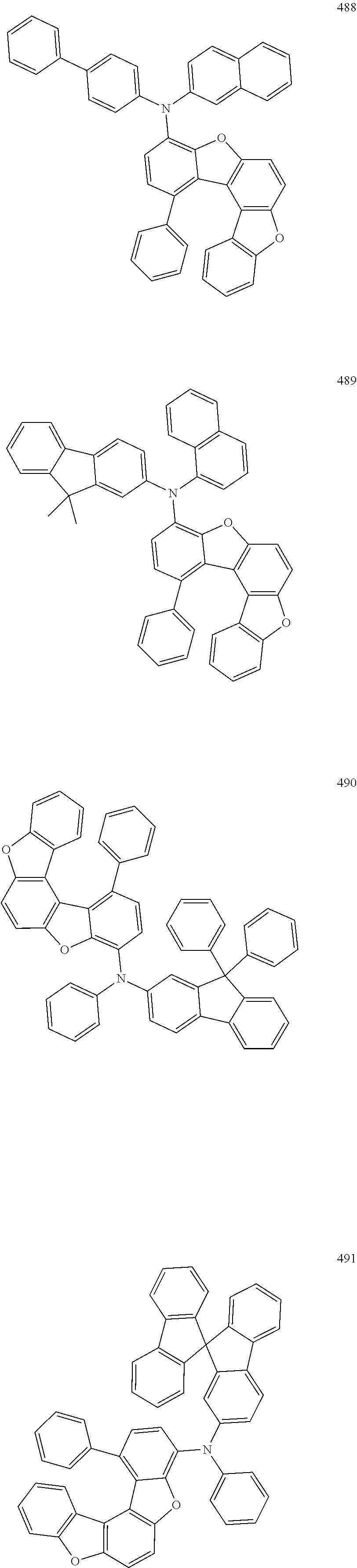 [SMILES: c1ccc(-c2ccc(N(c3ccc4ccccc4c3)c3ccc(-c4ccccc4)c4c3oc3ccc5oc6ccccc6c5c34)cc2)cc1 ; CC1(C)c2ccccc2-c2ccc(N(c3cccc4ccccc34)c3ccc(-c4ccccc4)c4c3oc3ccc5oc6ccccc6c5c34)cc21 ; c1ccc(-c2ccc(N(c3ccccc3)c3ccc4c(c3)C(c3ccccc3)(c3ccccc3)c3ccccc3-4)c3oc4ccc5oc6ccccc6c5c4c23)cc1 ; c1ccc(-c2ccc(N(c3ccccc3)c3ccc4c(c3)C3(c5ccccc5-c5ccccc53)c3ccccc3-4)c3oc4ccc5oc6ccccc6c5c4c23)cc1]

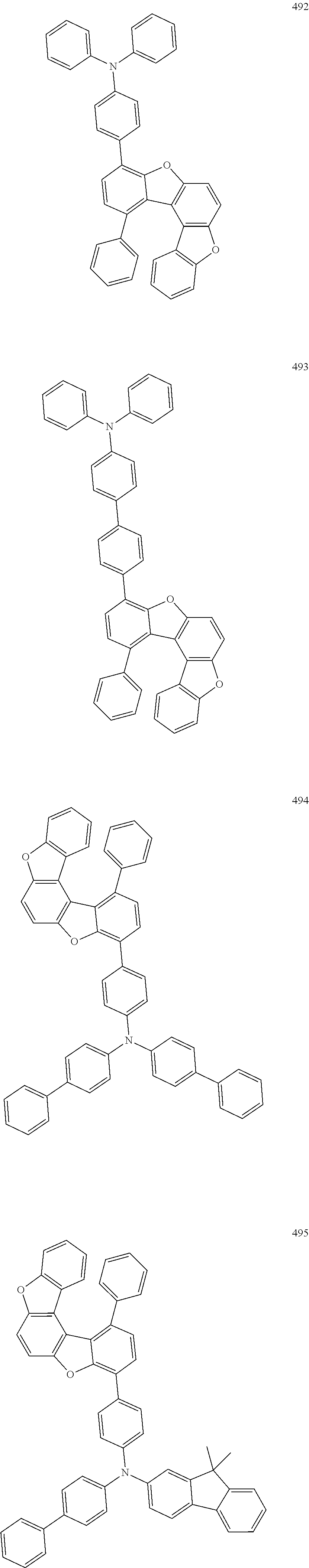 [SMILES: c1ccc(-c2ccc(-c3ccc(N(c4ccccc4)c4ccccc4)cc3)c3oc4ccc5oc6ccccc6c5c4c23)cc1 ; c1ccc(-c2ccc(-c3ccc(-c4ccc(N(c5ccccc5)c5ccccc5)cc4)cc3)c3oc4ccc5oc6ccccc6c5c4c23)cc1 ; c1ccc(-c2ccc(N(c3ccc(-c4ccccc4)cc3)c3ccc(-c4ccc(-c5ccccc5)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5ccccc5)c5c4oc4ccc6oc7ccccc7c6c45)cc3)cc21]

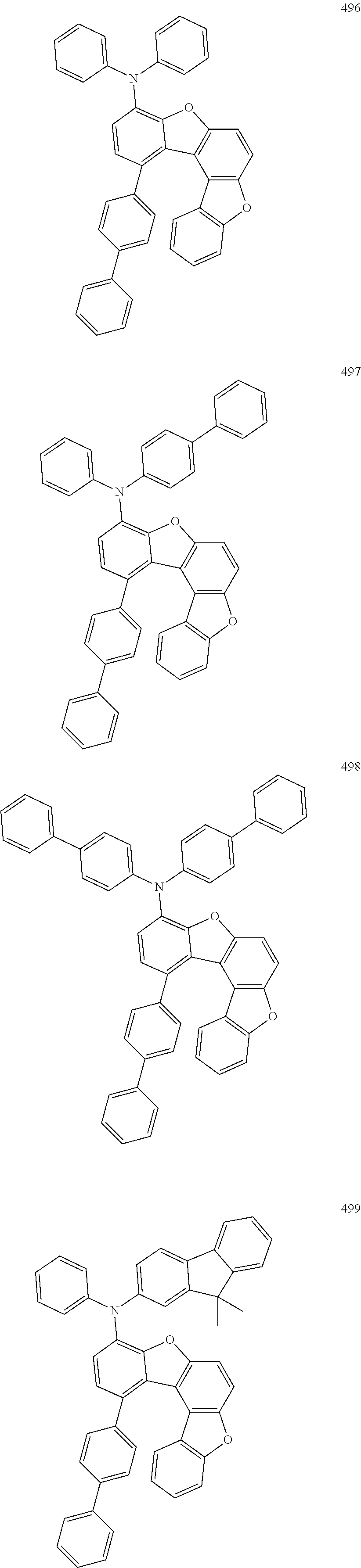 [SMILES: c1ccc(-c2ccc(-c3ccc(N(c4ccccc4)c4ccccc4)c4oc5ccc6oc7ccccc7c6c5c34)cc2)cc1 ; c1ccc(-c2ccc(-c3ccc(N(c4ccccc4)c4ccc(-c5ccccc5)cc4)c4oc5ccc6oc7ccccc7c6c5c34)cc2)cc1 ; c1ccc(-c2ccc(-c3ccc(N(c4ccc(-c5ccccc5)cc4)c4ccc(-c5ccccc5)cc4)c4oc5ccc6oc7ccccc7c6c5c34)cc2)cc1 ; CC1(C)c2ccccc2-c2ccc(N(c3ccccc3)c3ccc(-c4ccc(-c5ccccc5)cc4)c4c3oc3ccc5oc6ccccc6c5c34)cc21]

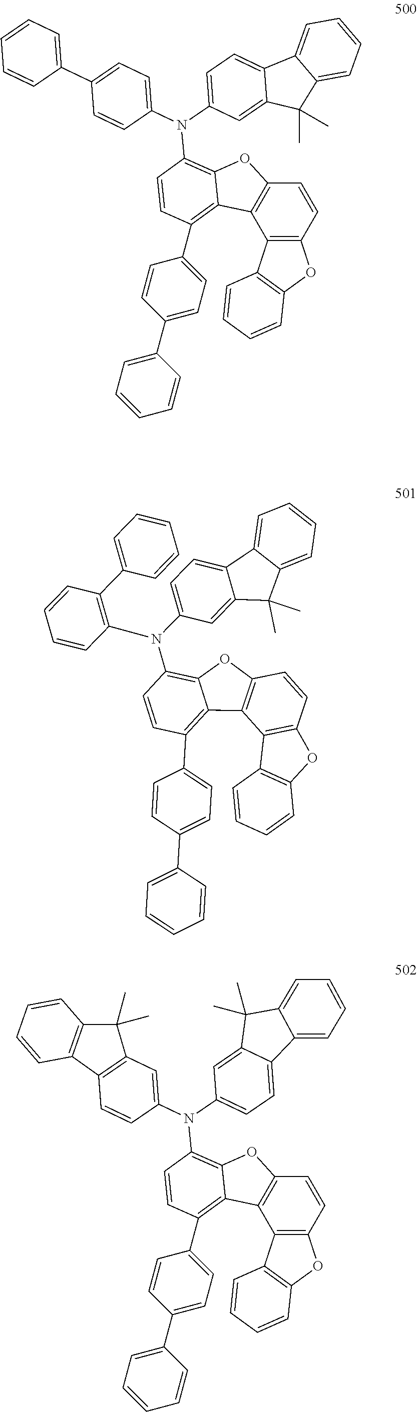 [SMILES: CC1(C)c2ccccc2-c2ccc(N(c3ccc(-c4ccccc4)cc3)c3ccc(-c4ccc(-c5ccccc5)cc4)c4c3oc3ccc5oc6ccccc6c5c34)cc21 ; CC1(C)c2ccccc2-c2ccc(N(c3ccccc3-c3ccccc3)c3ccc(-c4ccc(-c5ccccc5)cc4)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4ccc(-c5ccccc5)cc4)c4c3oc3ccc5oc6ccccc6c5c34)cc21]

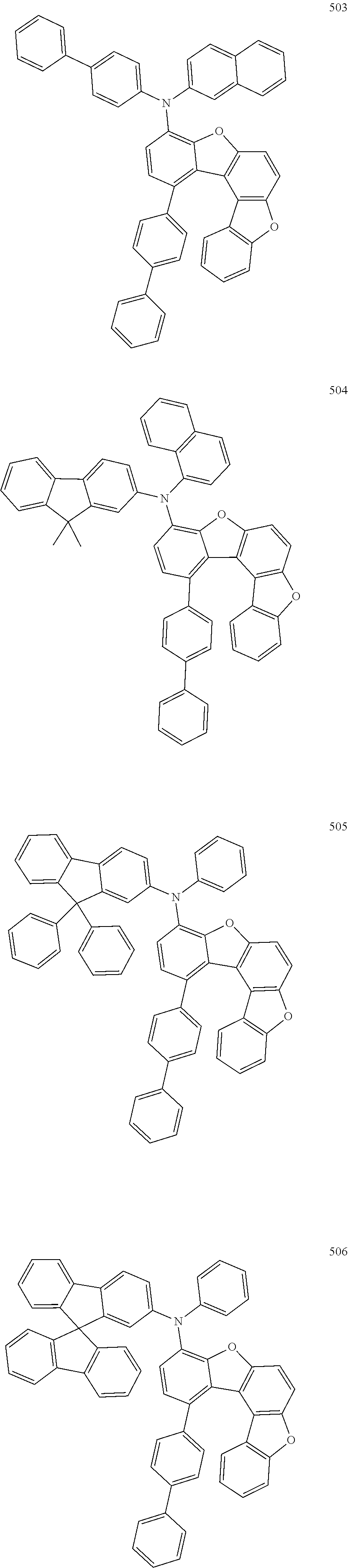 [SMILES: c1ccc(-c2ccc(-c3ccc(N(c4ccc(-c5ccccc5)cc4)c4ccc5ccccc5c4)c4oc5ccc6oc7ccccc7c6c5c34)cc2)cc1 ; CC1(C)c2ccccc2-c2ccc(N(c3cccc4ccccc34)c3ccc(-c4ccc(-c5ccccc5)cc4)c4c3oc3ccc5oc6ccccc6c5c34)cc21 ; c1ccc(-c2ccc(-c3ccc(N(c4ccccc4)c4ccc5c(c4)C(c4ccccc4)(c4ccccc4)c4ccccc4-5)c4oc5ccc6oc7ccccc7c6c5c34)cc2)cc1 ; c1ccc(-c2ccc(-c3ccc(N(c4ccccc4)c4ccc5c(c4)C4(c6ccccc6-c6ccccc64)c4ccccc4-5)c4oc5ccc6oc7ccccc7c6c5c34)cc2)cc1]

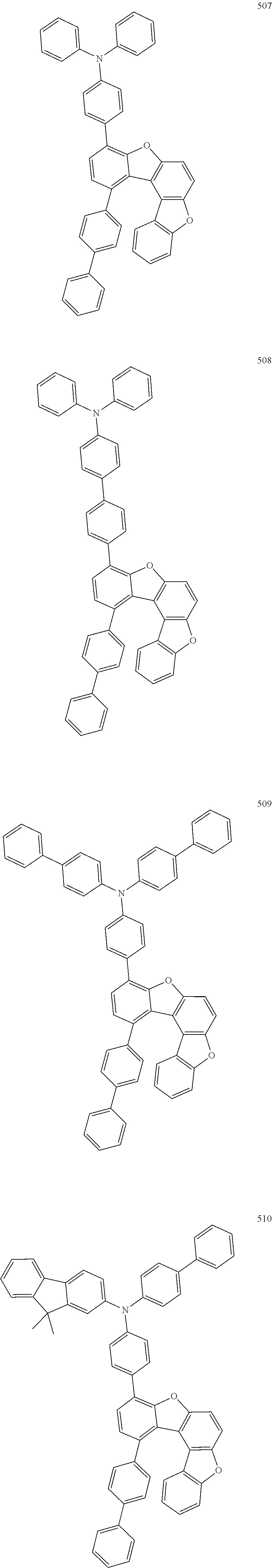 [SMILES: c1ccc(-c2ccc(-c3ccc(-c4ccc(N(c5ccccc5)c5ccccc5)cc4)c4oc5ccc6oc7ccccc7c6c5c34)cc2)cc1 ; c1ccc(-c2ccc(-c3ccc(-c4ccc(-c5ccc(N(c6ccccc6)c6ccccc6)cc5)cc4)c4oc5ccc6oc7ccccc7c6c5c34)cc2)cc1 ; c1ccc(-c2ccc(-c3ccc(-c4ccc(N(c5ccc(-c6ccccc6)cc5)c5ccc(-c6ccccc6)cc5)cc4)c4oc5ccc6oc7ccccc7c6c5c34)cc2)cc1 ; CC1(C)c2ccccc2-c2ccc(N(c3ccc(-c4ccccc4)cc3)c3ccc(-c4ccc(-c5ccc(-c6ccccc6)cc5)c5c4oc4ccc6oc7ccccc7c6c45)cc3)cc21]

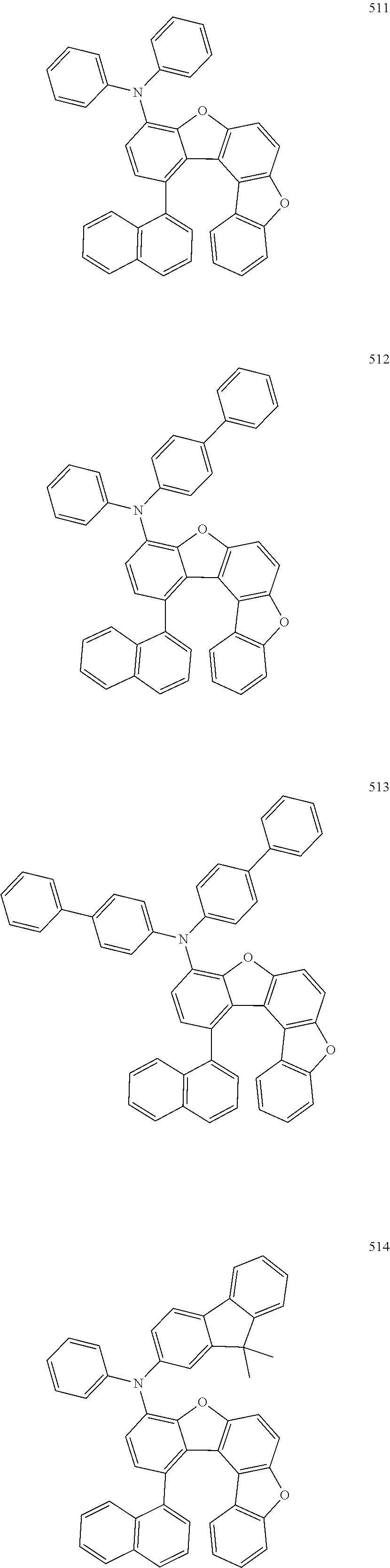 [SMILES: c1ccc(N(c2ccccc2)c2ccc(-c3cccc4ccccc34)c3c2oc2ccc4oc5ccccc5c4c23)cc1 ; c1ccc(-c2ccc(N(c3ccccc3)c3ccc(-c4cccc5ccccc45)c4c3oc3ccc5oc6ccccc6c5c34)cc2)cc1 ; c1ccc(-c2ccc(N(c3ccc(-c4ccccc4)cc3)c3ccc(-c4cccc5ccccc45)c4c3oc3ccc5oc6ccccc6c5c34)cc2)cc1 ; CC1(C)c2ccccc2-c2ccc(N(c3ccccc3)c3ccc(-c4cccc5ccccc45)c4c3oc3ccc5oc6ccccc6c5c34)cc21]

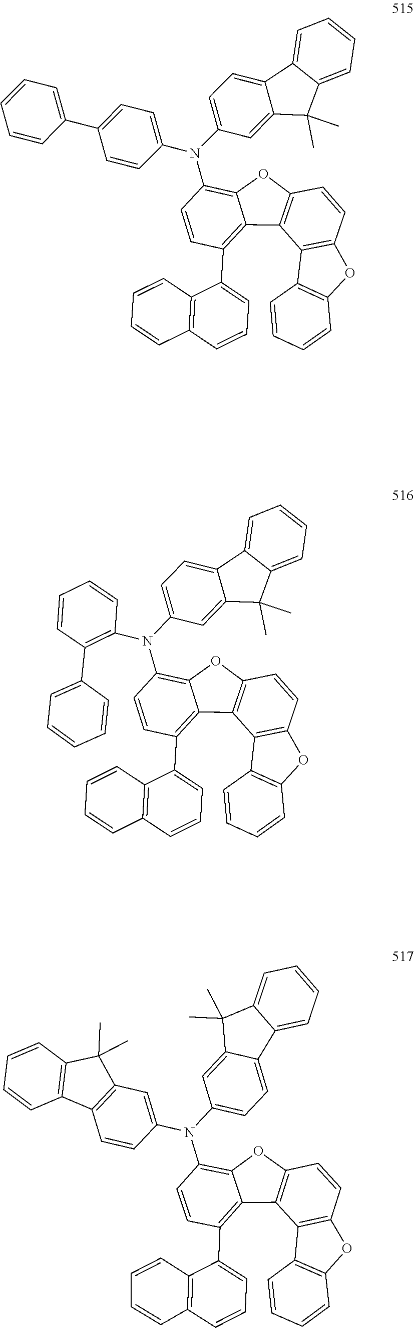 [SMILES: CC1(C)c2ccccc2-c2ccc(N(c3ccc(-c4ccccc4)cc3)c3ccc(-c4cccc5ccccc45)c4c3oc3ccc5oc6ccccc6c5c34)cc21 ; CC1(C)c2ccccc2-c2ccc(N(c3ccccc3-c3ccccc3)c3ccc(-c4cccc5ccccc45)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4cccc5ccccc45)c4c3oc3ccc5oc6ccccc6c5c34)cc21]

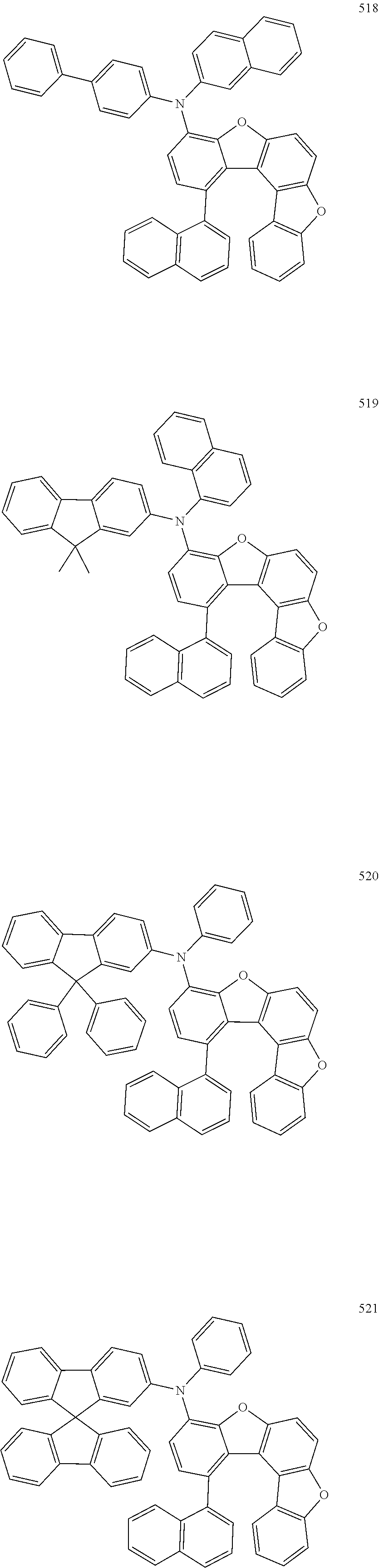 [SMILES: c1ccc(-c2ccc(N(c3ccc4ccccc4c3)c3ccc(-c4cccc5ccccc45)c4c3oc3ccc5oc6ccccc6c5c34)cc2)cc1 ; CC1(C)c2ccccc2-c2ccc(N(c3cccc4ccccc34)c3ccc(-c4cccc5ccccc45)c4c3oc3ccc5oc6ccccc6c5c34)cc21 ; c1ccc(N(c2ccc3c(c2)C(c2ccccc2)(c2ccccc2)c2ccccc2-3)c2ccc(-c3cccc4ccccc34)c3c2oc2ccc4oc5ccccc5c4c23)cc1 ; c1ccc(N(c2ccc3c(c2)C2(c4ccccc4-c4ccccc42)c2ccccc2-3)c2ccc(-c3cccc4ccccc34)c3c2oc2ccc4oc5ccccc5c4c23)cc1]

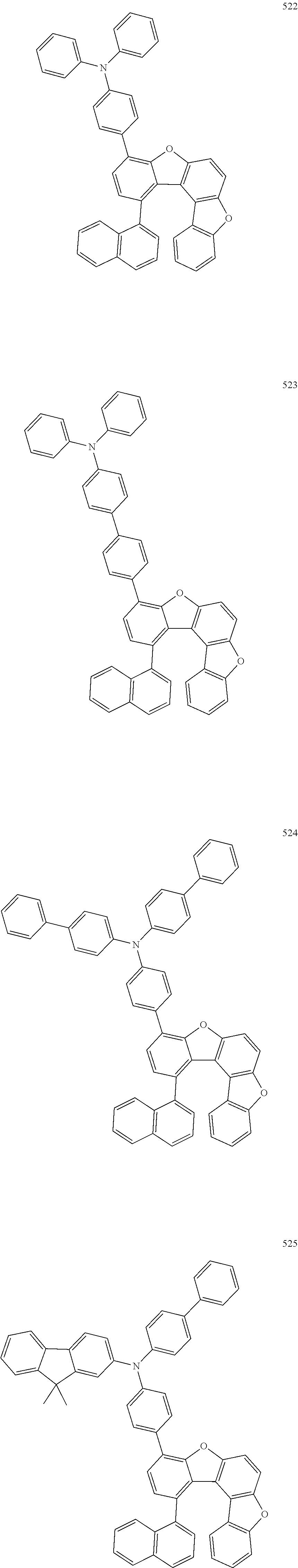 [SMILES: c1ccc(N(c2ccccc2)c2ccc(-c3ccc(-c4cccc5ccccc45)c4c3oc3ccc5oc6ccccc6c5c34)cc2)cc1 ; c1ccc(N(c2ccccc2)c2ccc(-c3ccc(-c4ccc(-c5cccc6ccccc56)c5c4oc4ccc6oc7ccccc7c6c45)cc3)cc2)cc1 ; c1ccc(-c2ccc(N(c3ccc(-c4ccccc4)cc3)c3ccc(-c4ccc(-c5cccc6ccccc56)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5cccc6ccccc56)c5c4oc4ccc6oc7ccccc7c6c45)cc3)cc21]

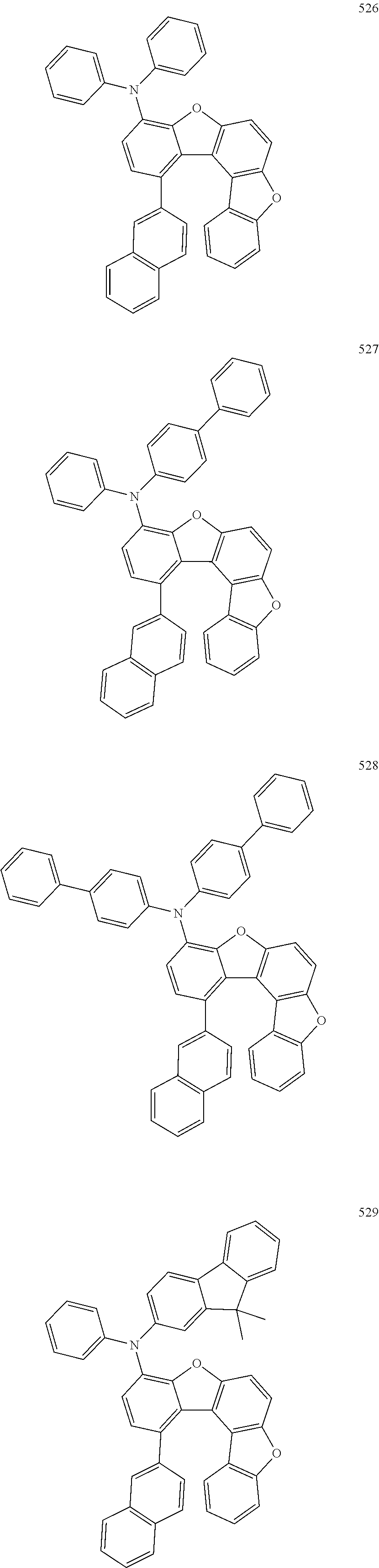 [SMILES: c1ccc(N(c2ccccc2)c2ccc(-c3ccc4ccccc4c3)c3c2oc2ccc4oc5ccccc5c4c23)cc1 ; c1ccc(-c2ccc(N(c3ccccc3)c3ccc(-c4ccc5ccccc5c4)c4c3oc3ccc5oc6ccccc6c5c34)cc2)cc1 ; c1ccc(-c2ccc(N(c3ccc(-c4ccccc4)cc3)c3ccc(-c4ccc5ccccc5c4)c4c3oc3ccc5oc6ccccc6c5c34)cc2)cc1 ; CC1(C)c2ccccc2-c2ccc(N(c3ccccc3)c3ccc(-c4ccc5ccccc5c4)c4c3oc3ccc5oc6ccccc6c5c34)cc21]

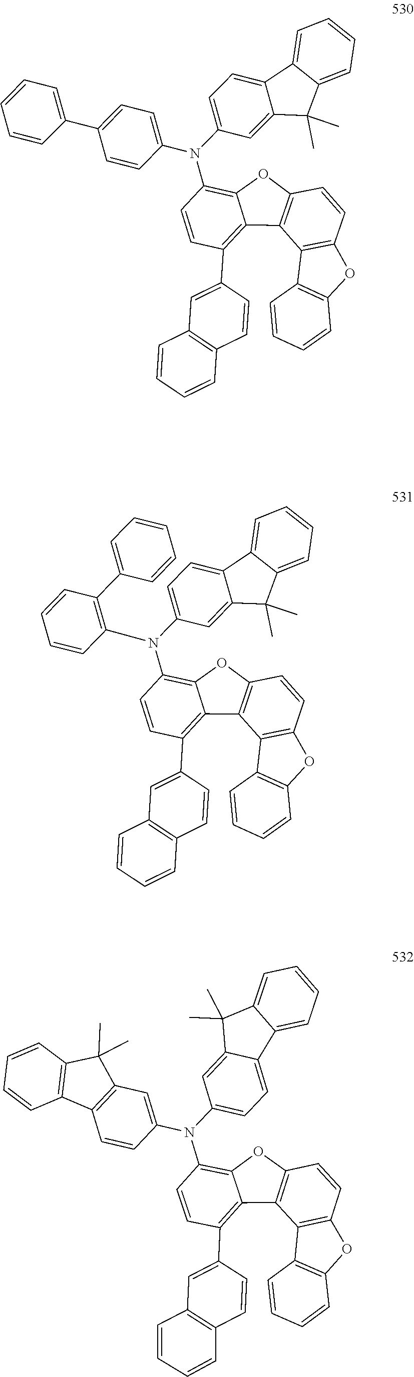 [SMILES: CC1(C)c2ccccc2-c2ccc(N(c3ccc(-c4ccccc4)cc3)c3ccc(-c4ccc5ccccc5c4)c4c3oc3ccc5oc6ccccc6c5c34)cc21 ; CC1(C)c2ccccc2-c2ccc(N(c3ccccc3-c3ccccc3)c3ccc(-c4ccc5ccccc5c4)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4ccc5ccccc5c4)c4c3oc3ccc5oc6ccccc6c5c34)cc21]

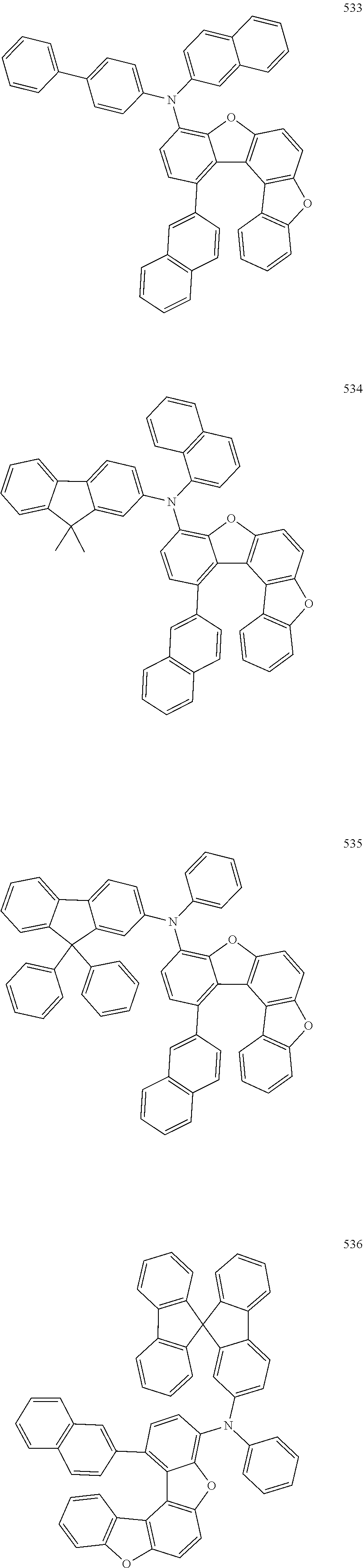 [SMILES: c1ccc(-c2ccc(N(c3ccc4ccccc4c3)c3ccc(-c4ccc5ccccc5c4)c4c3oc3ccc5oc6ccccc6c5c34)cc2)cc1 ; CC1(C)c2ccccc2-c2ccc(N(c3cccc4ccccc34)c3ccc(-c4ccc5ccccc5c4)c4c3oc3ccc5oc6ccccc6c5c34)cc21 ; c1ccc(N(c2ccc3c(c2)C(c2ccccc2)(c2ccccc2)c2ccccc2-3)c2ccc(-c3ccc4ccccc4c3)c3c2oc2ccc4oc5ccccc5c4c23)cc1 ; c1ccc(N(c2ccc3c(c2)C2(c4ccccc4-c4ccccc42)c2ccccc2-3)c2ccc(-c3ccc4ccccc4c3)c3c2oc2ccc4oc5ccccc5c4c23)cc1]

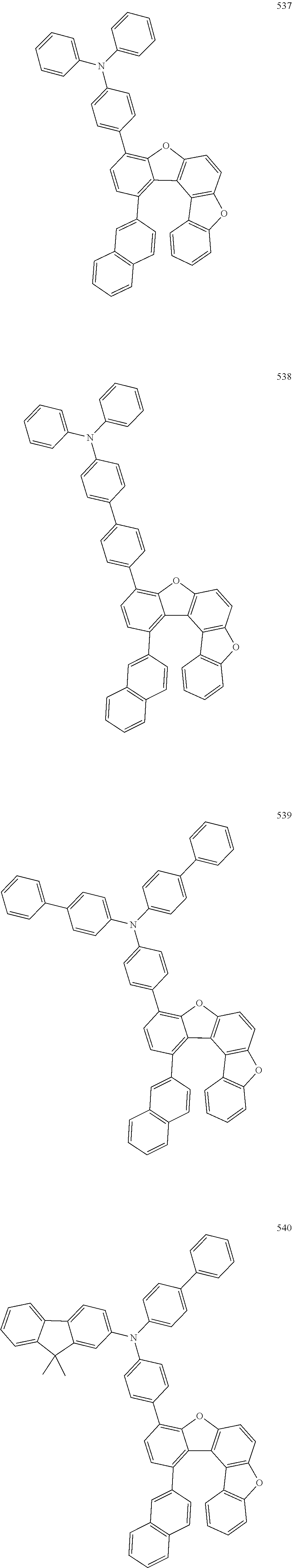 [SMILES: c1ccc(N(c2ccccc2)c2ccc(-c3ccc(-c4ccc5ccccc5c4)c4c3oc3ccc5oc6ccccc6c5c34)cc2)cc1 ; c1ccc(N(c2ccccc2)c2ccc(-c3ccc(-c4ccc(-c5ccc6ccccc6c5)c5c4oc4ccc6oc7ccccc7c6c45)cc3)cc2)cc1 ; c1ccc(-c2ccc(N(c3ccc(-c4ccccc4)cc3)c3ccc(-c4ccc(-c5ccc6ccccc6c5)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5ccc6ccccc6c5)c5c4oc4ccc6oc7ccccc7c6c45)cc3)cc21]

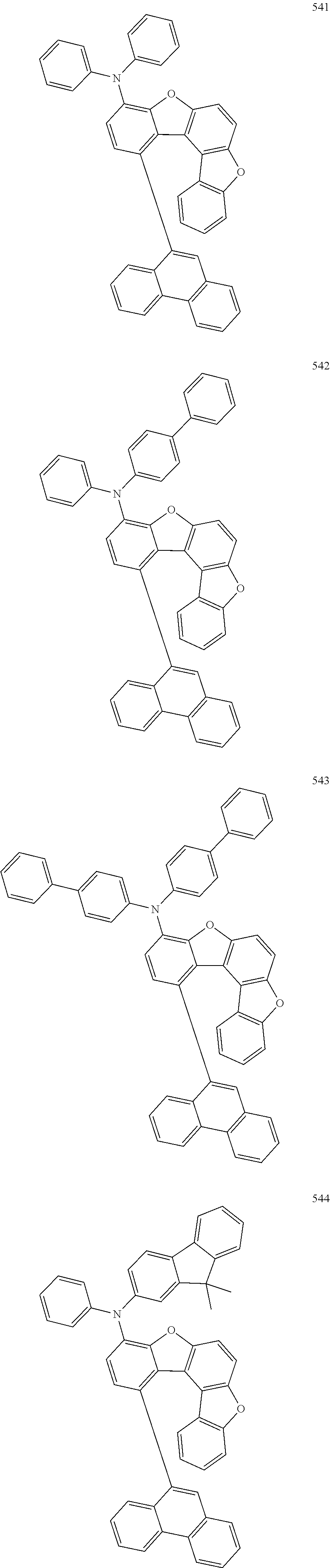 [SMILES: c1ccc(N(c2ccccc2)c2ccc(-c3cc4ccccc4c4ccccc34)c3c2oc2ccc4oc5ccccc5c4c23)cc1 ; c1ccc(-c2ccc(N(c3ccccc3)c3ccc(-c4cc5ccccc5c5ccccc45)c4c3oc3ccc5oc6ccccc6c5c34)cc2)cc1 ; c1ccc(-c2ccc(N(c3ccc(-c4ccccc4)cc3)c3ccc(-c4cc5ccccc5c5ccccc45)c4c3oc3ccc5oc6ccccc6c5c34)cc2)cc1 ; CC1(C)c2ccccc2-c2ccc(N(c3ccccc3)c3ccc(-c4cc5ccccc5c5ccccc45)c4c3oc3ccc5oc6ccccc6c5c34)cc21]

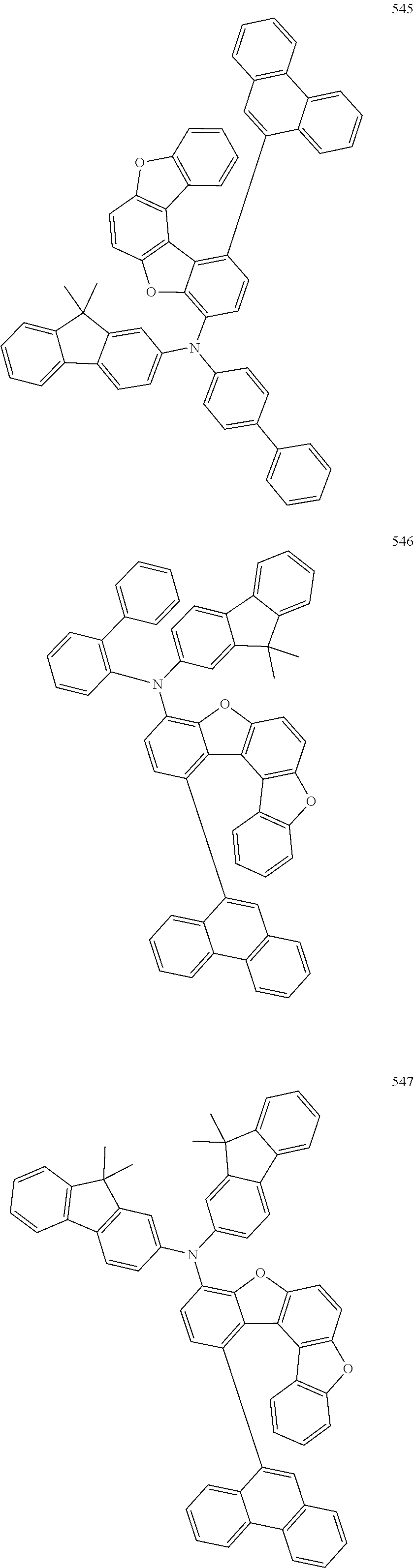

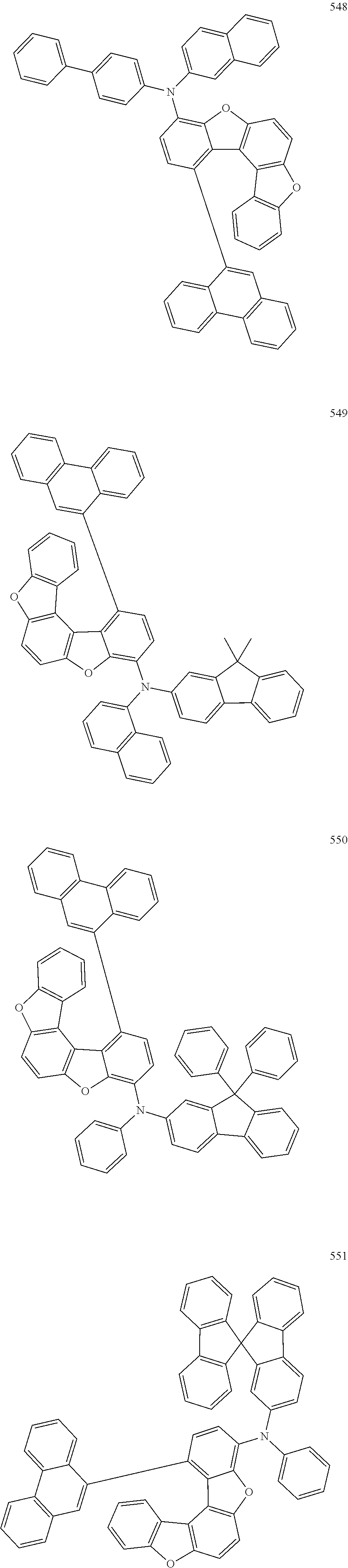 [SMILES: c1ccc(-c2ccc(N(c3ccc4ccccc4c3)c3ccc(-c4cc5ccccc5c5ccccc45)c4c3oc3ccc5oc6ccccc6c5c34)cc2)cc1 ; CC1(C)c2ccccc2-c2ccc(N(c3cccc4ccccc34)c3ccc(-c4cc5ccccc5c5ccccc45)c4c3oc3ccc5oc6ccccc6c5c34)cc21 ; c1ccc(N(c2ccc3c(c2)C(c2ccccc2)(c2ccccc2)c2ccccc2-3)c2ccc(-c3cc4ccccc4c4ccccc34)c3c2oc2ccc4oc5ccccc5c4c23)cc1 ; c1ccc(N(c2ccc3c(c2)C2(c4ccccc4-c4ccccc42)c2ccccc2-3)c2ccc(-c3cc4ccccc4c4ccccc34)c3c2oc2ccc4oc5ccccc5c4c23)cc1]

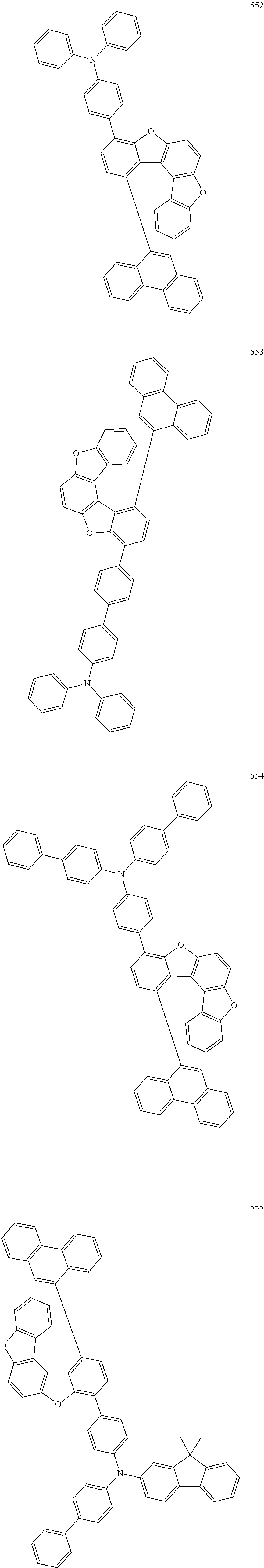 [SMILES: c1ccc(N(c2ccccc2)c2ccc(-c3ccc(-c4cc5ccccc5c5ccccc45)c4c3oc3ccc5oc6ccccc6c5c34)cc2)cc1 ; c1ccc(N(c2ccccc2)c2ccc(-c3ccc(-c4ccc(-c5cc6ccccc6c6ccccc56)c5c4oc4ccc6oc7ccccc7c6c45)cc3)cc2)cc1 ; c1ccc(-c2ccc(N(c3ccc(-c4ccccc4)cc3)c3ccc(-c4ccc(-c5cc6ccccc6c6ccccc56)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5cc6ccccc6c6ccccc56)c5c4oc4ccc6oc7ccccc7c6c45)cc3)cc21]

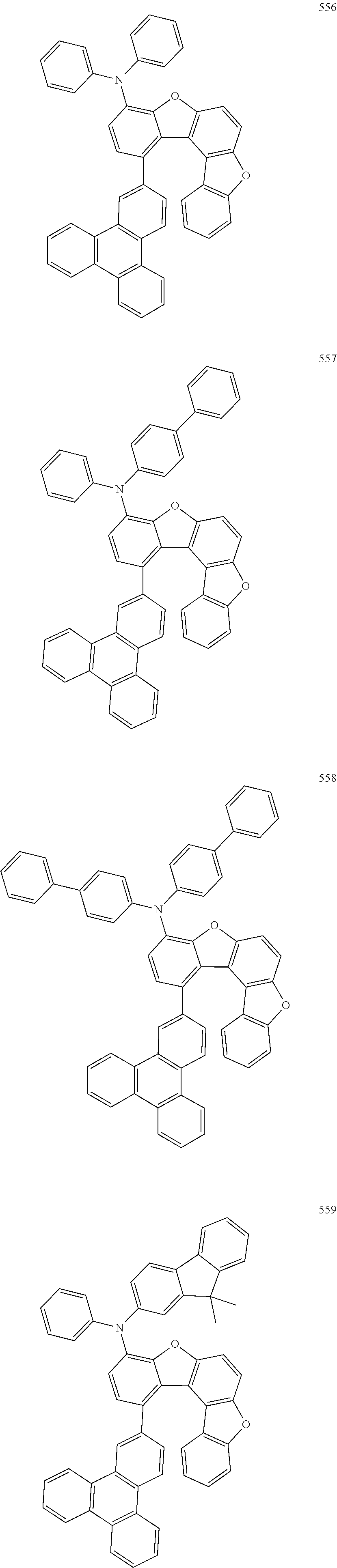 [SMILES: c1ccc(N(c2ccccc2)c2ccc(-c3ccc4c5ccccc5c5ccccc5c4c3)c3c2oc2ccc4oc5ccccc5c4c23)cc1 ; c1ccc(-c2ccc(N(c3ccccc3)c3ccc(-c4ccc5c6ccccc6c6ccccc6c5c4)c4c3oc3ccc5oc6ccccc6c5c34)cc2)cc1 ; c1ccc(-c2ccc(N(c3ccc(-c4ccccc4)cc3)c3ccc(-c4ccc5c6ccccc6c6ccccc6c5c4)c4c3oc3ccc5oc6ccccc6c5c34)cc2)cc1 ; CC1(C)c2ccccc2-c2ccc(N(c3ccccc3)c3ccc(-c4ccc5c6ccccc6c6ccccc6c5c4)c4c3oc3ccc5oc6ccccc6c5c34)cc21]

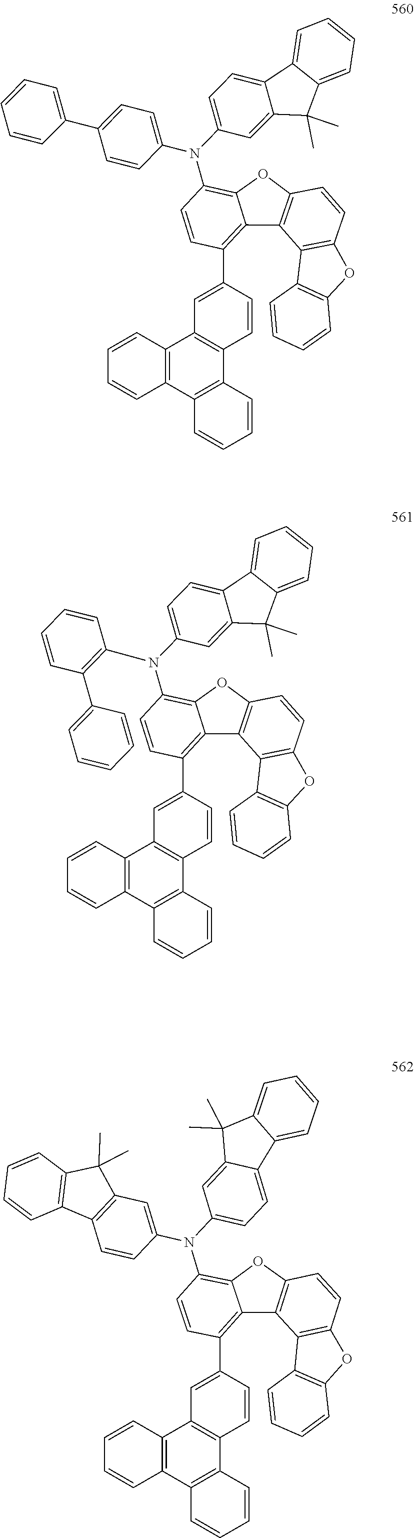 [SMILES: CC1(C)c2ccccc2-c2ccc(N(c3ccc(-c4ccccc4)cc3)c3ccc(-c4ccc5c6ccccc6c6ccccc6c5c4)c4c3oc3ccc5oc6ccccc6c5c34)cc21 ; CC1(C)c2ccccc2-c2ccc(N(c3ccccc3-c3ccccc3)c3ccc(-c4ccc5c6ccccc6c6ccccc6c5c4)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4ccc5c6ccccc6c6ccccc6c5c4)c4c3oc3ccc5oc6ccccc6c5c34)cc21]

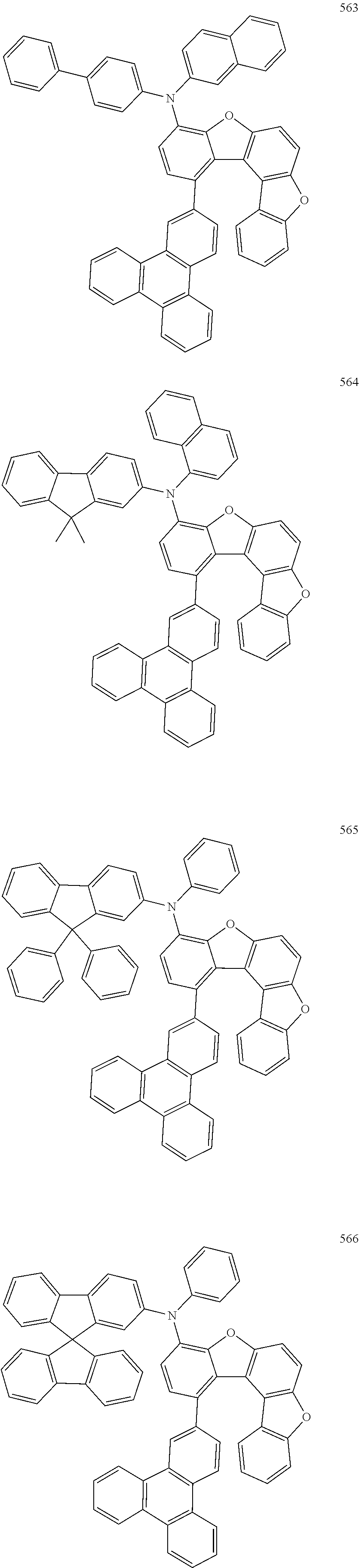 [SMILES: c1ccc(-c2ccc(N(c3ccc4ccccc4c3)c3ccc(-c4ccc5c6ccccc6c6ccccc6c5c4)c4c3oc3ccc5oc6ccccc6c5c34)cc2)cc1 ; CC1(C)c2ccccc2-c2ccc(N(c3cccc4ccccc34)c3ccc(-c4ccc5c6ccccc6c6ccccc6c5c4)c4c3oc3ccc5oc6ccccc6c5c34)cc21 ; c1ccc(N(c2ccc3c(c2)C(c2ccccc2)(c2ccccc2)c2ccccc2-3)c2ccc(-c3ccc4c5ccccc5c5ccccc5c4c3)c3c2oc2ccc4oc5ccccc5c4c23)cc1 ; c1ccc(N(c2ccc3c(c2)C2(c4ccccc4-c4ccccc42)c2ccccc2-3)c2ccc(-c3ccc4c5ccccc5c5ccccc5c4c3)c3c2oc2ccc4oc5ccccc5c4c23)cc1]

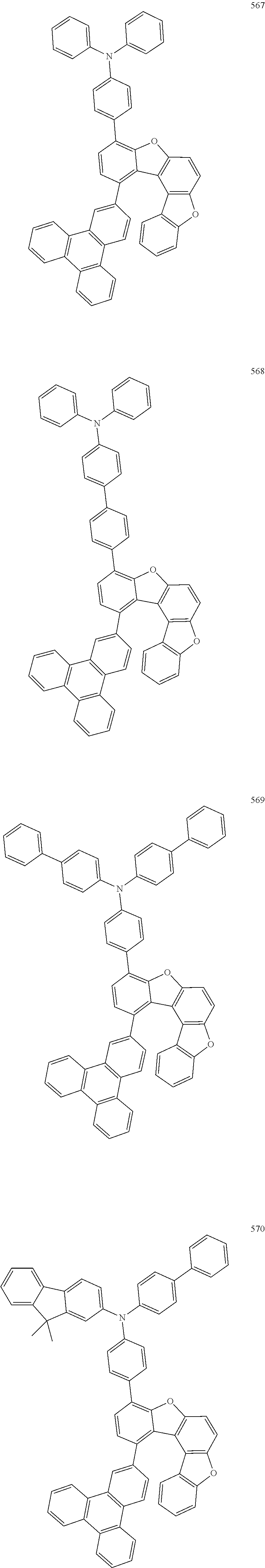 [SMILES: c1ccc(N(c2ccccc2)c2ccc(-c3ccc(-c4ccc5c6ccccc6c6ccccc6c5c4)c4c3oc3ccc5oc6ccccc6c5c34)cc2)cc1 ; c1ccc(N(c2ccccc2)c2ccc(-c3ccc(-c4ccc(-c5ccc6c7ccccc7c7ccccc7c6c5)c5c4oc4ccc6oc7ccccc7c6c45)cc3)cc2)cc1 ; c1ccc(-c2ccc(N(c3ccc(-c4ccccc4)cc3)c3ccc(-c4ccc(-c5ccc6c7ccccc7c7ccccc7c6c5)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5ccc6c7ccccc7c7ccccc7c6c5)c5c4oc4ccc6oc7ccccc7c6c45)cc3)cc21]

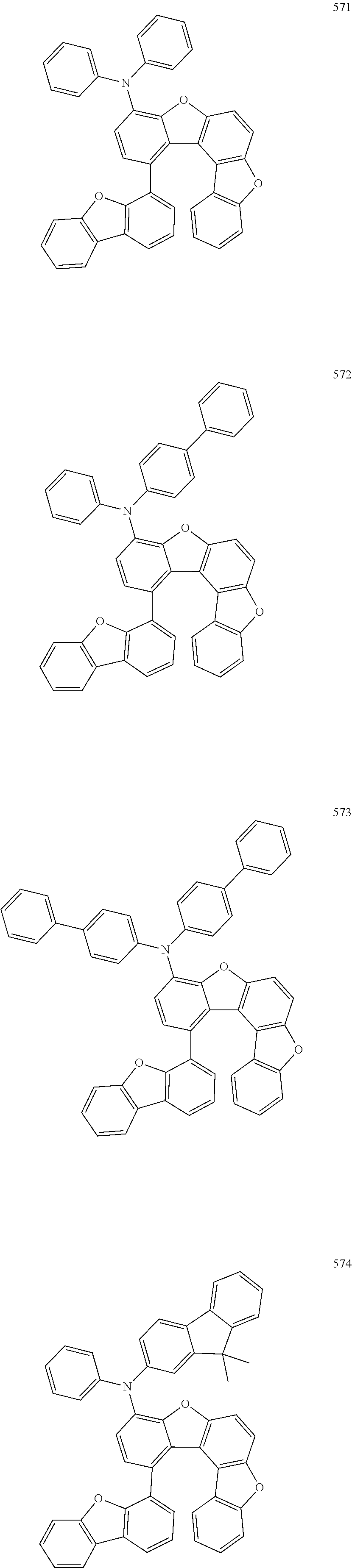 [SMILES: c1ccc(N(c2ccccc2)c2ccc(-c3cccc4c3oc3ccccc34)c3c2oc2ccc4oc5ccccc5c4c23)cc1 ; c1ccc(-c2ccc(N(c3ccccc3)c3ccc(-c4cccc5c4oc4ccccc45)c4c3oc3ccc5oc6ccccc6c5c34)cc2)cc1 ; c1ccc(-c2ccc(N(c3ccc(-c4ccccc4)cc3)c3ccc(-c4cccc5c4oc4ccccc45)c4c3oc3ccc5oc6ccccc6c5c34)cc2)cc1 ; CC1(C)c2ccccc2-c2ccc(N(c3ccccc3)c3ccc(-c4cccc5c4oc4ccccc45)c4c3oc3ccc5oc6ccccc6c5c34)cc21]

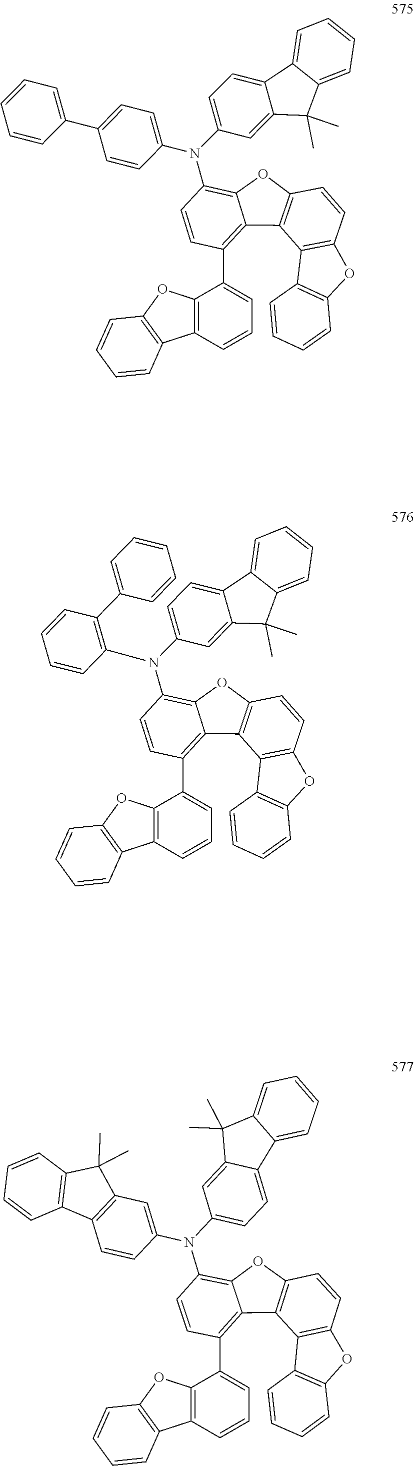 [SMILES: CC1(C)c2ccccc2-c2ccc(N(c3ccc(-c4ccccc4)cc3)c3ccc(-c4cccc5c4oc4ccccc45)c4c3oc3ccc5oc6ccccc6c5c34)cc21 ; CC1(C)c2ccccc2-c2ccc(N(c3ccccc3-c3ccccc3)c3ccc(-c4cccc5c4oc4ccccc45)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4cccc5c4oc4ccccc45)c4c3oc3ccc5oc6ccccc6c5c34)cc21]

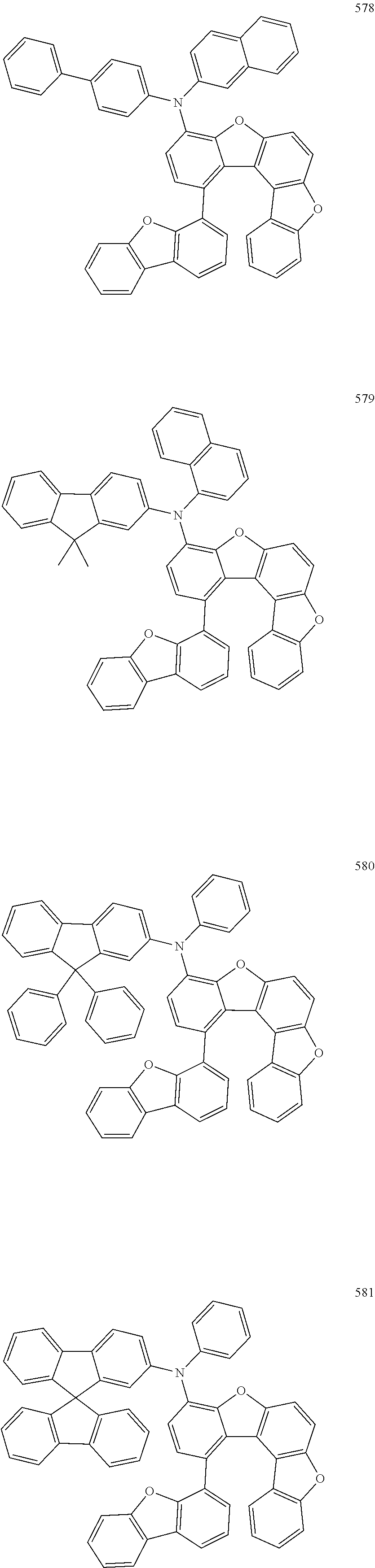 [SMILES: c1ccc(-c2ccc(N(c3ccc4ccccc4c3)c3ccc(-c4cccc5c4oc4ccccc45)c4c3oc3ccc5oc6ccccc6c5c34)cc2)cc1 ; CC1(C)c2ccccc2-c2ccc(N(c3cccc4ccccc34)c3ccc(-c4cccc5c4oc4ccccc45)c4c3oc3ccc5oc6ccccc6c5c34)cc21 ; c1ccc(N(c2ccc3c(c2)C(c2ccccc2)(c2ccccc2)c2ccccc2-3)c2ccc(-c3cccc4c3oc3ccccc34)c3c2oc2ccc4oc5ccccc5c4c23)cc1 ; c1ccc(N(c2ccc3c(c2)C2(c4ccccc4-c4ccccc42)c2ccccc2-3)c2ccc(-c3cccc4c3oc3ccccc34)c3c2oc2ccc4oc5ccccc5c4c23)cc1]

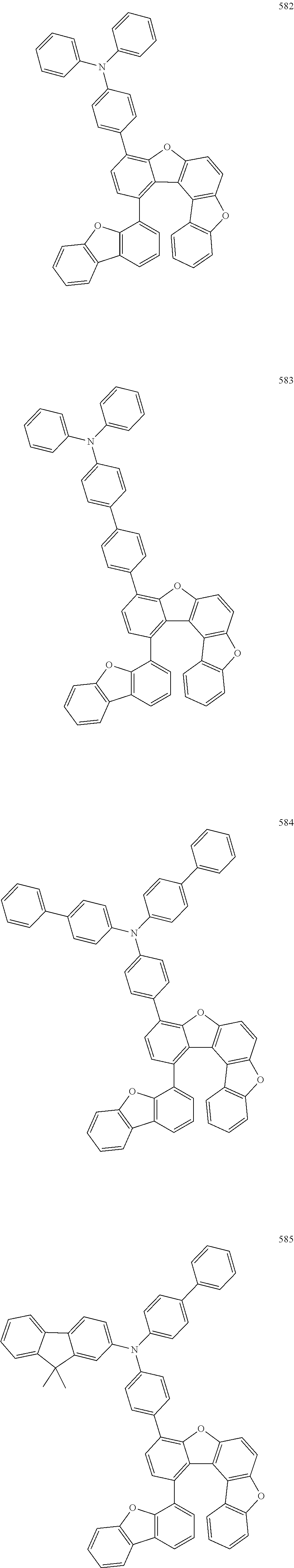 [SMILES: c1ccc(N(c2ccccc2)c2ccc(-c3ccc(-c4cccc5c4oc4ccccc45)c4c3oc3ccc5oc6ccccc6c5c34)cc2)cc1 ; c1ccc(N(c2ccccc2)c2ccc(-c3ccc(-c4ccc(-c5cccc6c5oc5ccccc56)c5c4oc4ccc6oc7ccccc7c6c45)cc3)cc2)cc1 ; c1ccc(-c2ccc(N(c3ccc(-c4ccccc4)cc3)c3ccc(-c4ccc(-c5cccc6c5oc5ccccc56)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5cccc6c5oc5ccccc56)c5c4oc4ccc6oc7ccccc7c6c45)cc3)cc21]

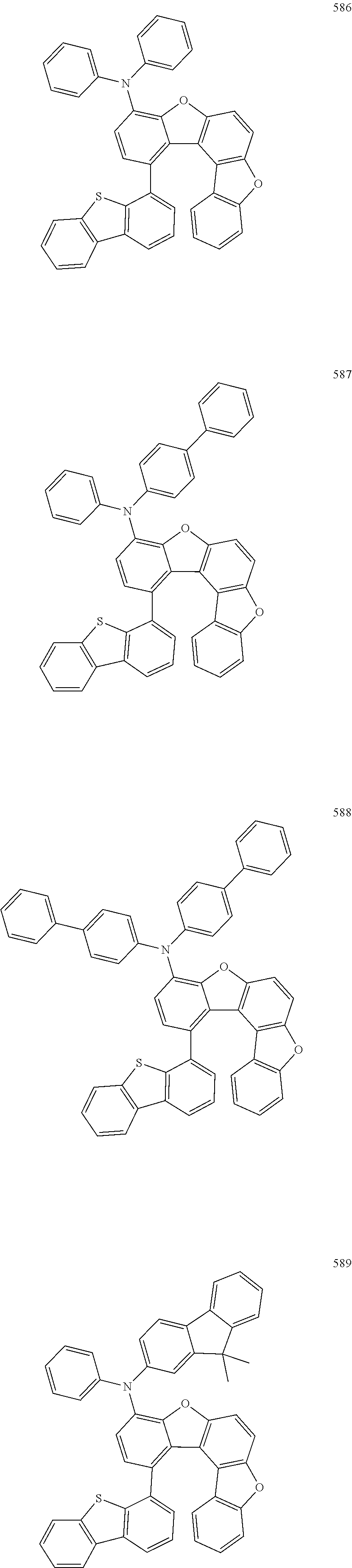 [SMILES: c1ccc(N(c2ccccc2)c2ccc(-c3cccc4c3sc3ccccc34)c3c2oc2ccc4oc5ccccc5c4c23)cc1 ; c1ccc(-c2ccc(N(c3ccccc3)c3ccc(-c4cccc5c4sc4ccccc45)c4c3oc3ccc5oc6ccccc6c5c34)cc2)cc1 ; c1ccc(-c2ccc(N(c3ccc(-c4ccccc4)cc3)c3ccc(-c4cccc5c4sc4ccccc45)c4c3oc3ccc5oc6ccccc6c5c34)cc2)cc1 ; CC1(C)c2ccccc2-c2ccc(N(c3ccccc3)c3ccc(-c4cccc5c4sc4ccccc45)c4c3oc3ccc5oc6ccccc6c5c34)cc21]

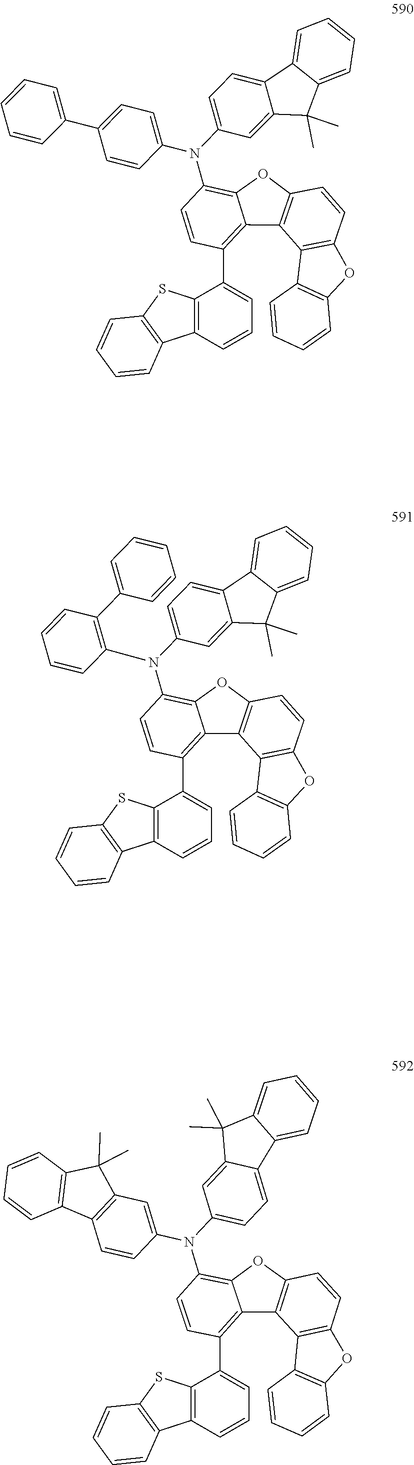 [SMILES: CC1(C)c2ccccc2-c2ccc(N(c3ccc(-c4ccccc4)cc3)c3ccc(-c4cccc5c4sc4ccccc45)c4c3oc3ccc5oc6ccccc6c5c34)cc21 ; CC1(C)c2ccccc2-c2ccc(N(c3ccccc3-c3ccccc3)c3ccc(-c4cccc5c4sc4ccccc45)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4cccc5c4sc4ccccc45)c4c3oc3ccc5oc6ccccc6c5c34)cc21]

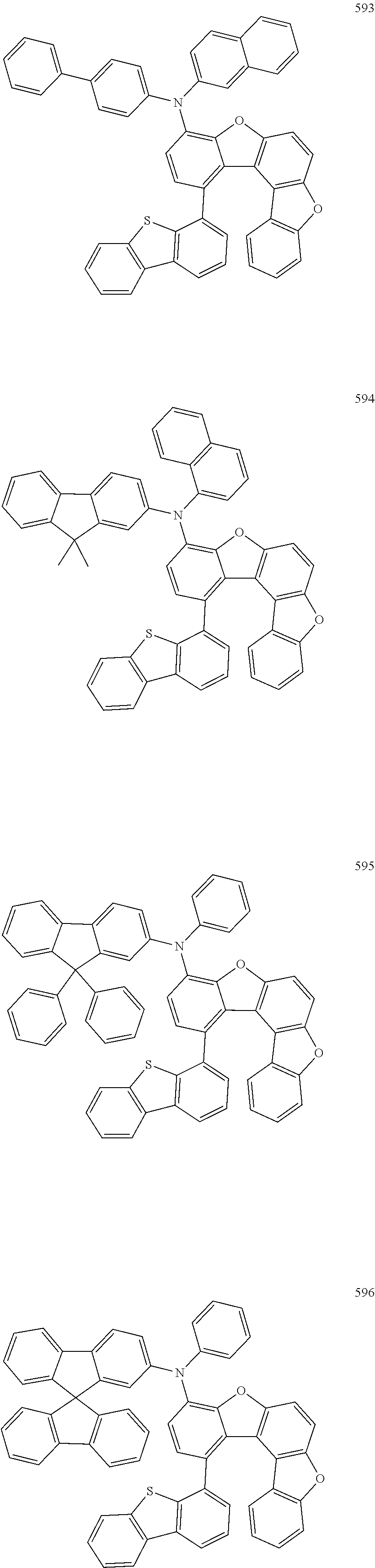 [SMILES: c1ccc(-c2ccc(N(c3ccc4ccccc4c3)c3ccc(-c4cccc5c4sc4ccccc45)c4c3oc3ccc5oc6ccccc6c5c34)cc2)cc1 ; CC1(C)c2ccccc2-c2ccc(N(c3cccc4ccccc34)c3ccc(-c4cccc5c4sc4ccccc45)c4c3oc3ccc5oc6ccccc6c5c34)cc21 ; c1ccc(N(c2ccc3c(c2)C(c2ccccc2)(c2ccccc2)c2ccccc2-3)c2ccc(-c3cccc4c3sc3ccccc34)c3c2oc2ccc4oc5ccccc5c4c23)cc1 ; c1ccc(N(c2ccc3c(c2)C2(c4ccccc4-c4ccccc42)c2ccccc2-3)c2ccc(-c3cccc4c3sc3ccccc34)c3c2oc2ccc4oc5ccccc5c4c23)cc1]

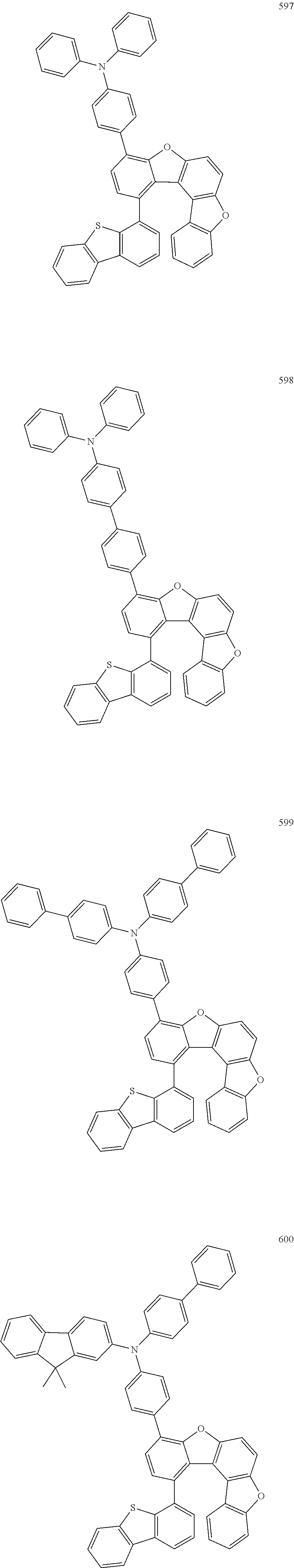 [SMILES: c1ccc(N(c2ccccc2)c2ccc(-c3ccc(-c4cccc5c4sc4ccccc45)c4c3oc3ccc5oc6ccccc6c5c34)cc2)cc1 ; c1ccc(N(c2ccccc2)c2ccc(-c3ccc(-c4ccc(-c5cccc6c5sc5ccccc56)c5c4oc4ccc6oc7ccccc7c6c45)cc3)cc2)cc1 ; c1ccc(-c2ccc(N(c3ccc(-c4ccccc4)cc3)c3ccc(-c4ccc(-c5cccc6c5sc5ccccc56)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5cccc6c5sc5ccccc56)c5c4oc4ccc6oc7ccccc7c6c45)cc3)cc21]

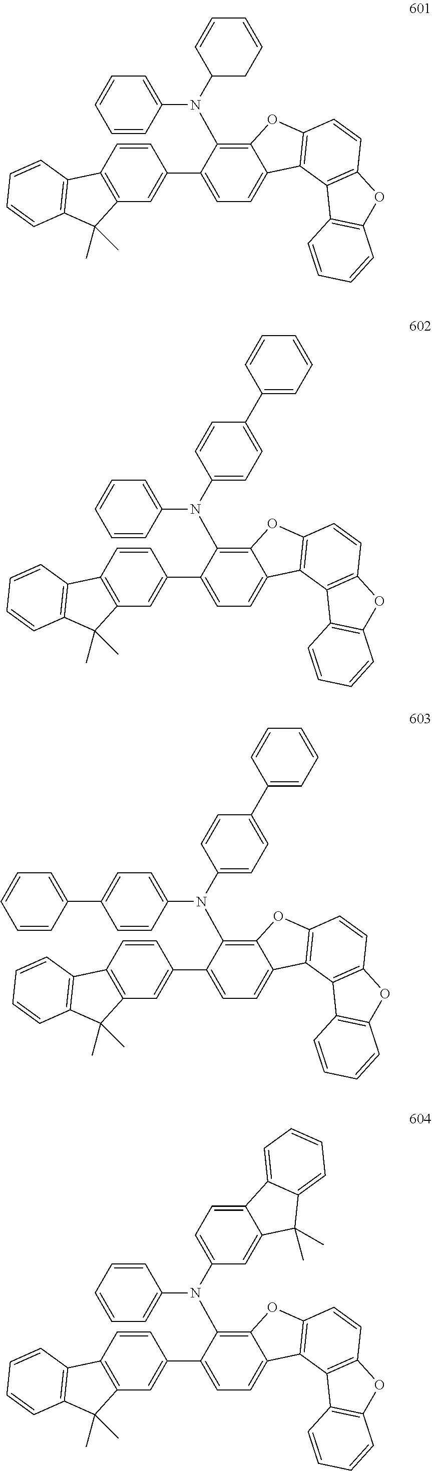 [SMILES: CC1(C)c2ccccc2-c2ccc(-c3ccc4c(oc5ccc6oc7ccccc7c6c54)c3N(c3ccccc3)C3C=CC=CC3)cc21 ; CC1(C)c2ccccc2-c2ccc(-c3ccc4c(oc5ccc6oc7ccccc7c6c54)c3N(c3ccccc3)c3ccc(-c4ccccc4)cc3)cc21 ; CC1(C)c2ccccc2-c2ccc(-c3ccc4c(oc5ccc6oc7ccccc7c6c54)c3N(c3ccc(-c4ccccc4)cc3)c3ccc(-c4ccccc4)cc3)cc21 ; CC1(C)c2ccccc2-c2ccc(-c3ccc4c(oc5ccc6oc7ccccc7c6c54)c3N(c3ccccc3)c3ccc4c(c3)C(C)(C)c3ccccc3-4)cc21]

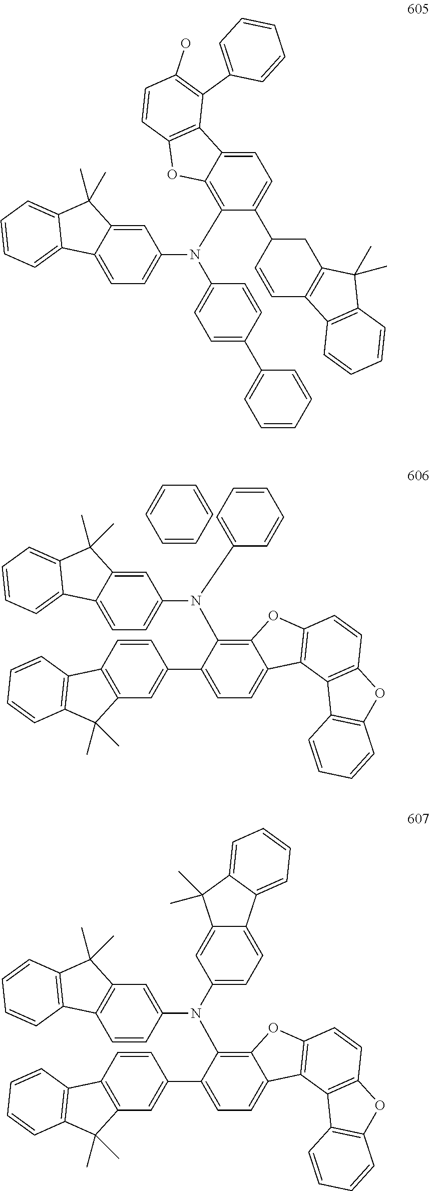

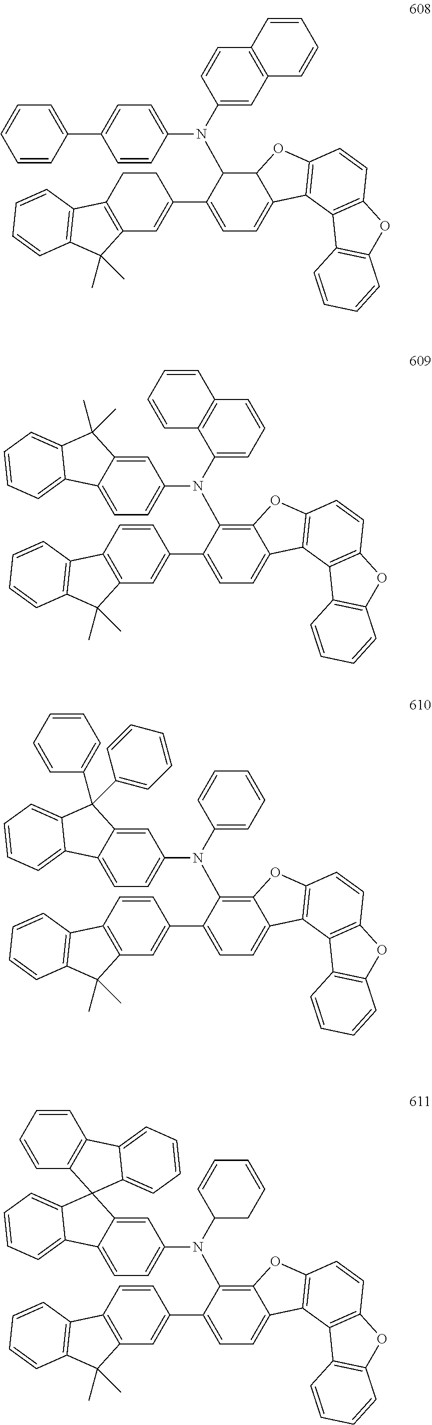 [SMILES: CC1(C)C2=C(CCC(C3=CC=C4c5c(ccc6oc7ccccc7c56)OC4C3N(c3ccc(-c4ccccc4)cc3)c3ccc4ccccc4c3)=C2)c2ccccc21 ; CC1(C)c2ccccc2-c2ccc(-c3ccc4c(oc5ccc6oc7ccccc7c6c54)c3N(c3ccc4c(c3)C(C)(C)c3ccccc3-4)c3cccc4ccccc34)cc21 ; CC1(C)c2ccccc2-c2ccc(-c3ccc4c(oc5ccc6oc7ccccc7c6c54)c3N(c3ccccc3)c3ccc4c(c3)C(c3ccccc3)(c3ccccc3)c3ccccc3-4)cc21 ; CC1(C)c2ccccc2-c2ccc(-c3ccc4c(oc5ccc6oc7ccccc7c6c54)c3N(c3ccc4c(c3)C3(c5ccccc5-c5ccccc53)c3ccccc3-4)C3C=CC=CC3)cc21]

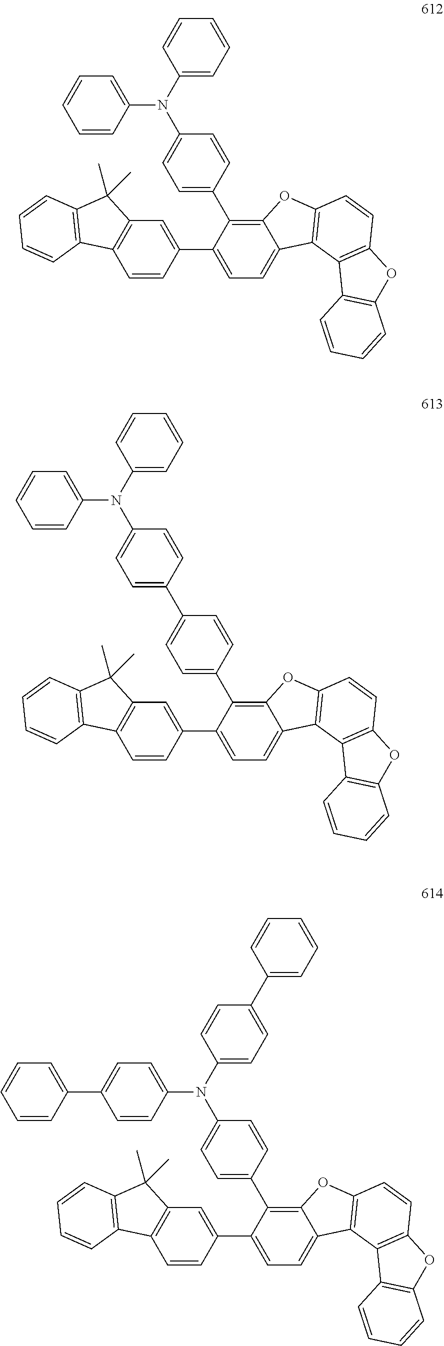 [SMILES: CC1(C)c2ccccc2-c2ccc(-c3ccc4c(oc5ccc6oc7ccccc7c6c54)c3-c3ccc(N(c4ccccc4)c4ccccc4)cc3)cc21 ; CC1(C)c2ccccc2-c2ccc(-c3ccc4c(oc5ccc6oc7ccccc7c6c54)c3-c3ccc(-c4ccc(N(c5ccccc5)c5ccccc5)cc4)cc3)cc21 ; CC1(C)c2ccccc2-c2ccc(-c3ccc4c(oc5ccc6oc7ccccc7c6c54)c3-c3ccc(N(c4ccc(-c5ccccc5)cc4)c4ccc(-c5ccccc5)cc4)cc3)cc21]

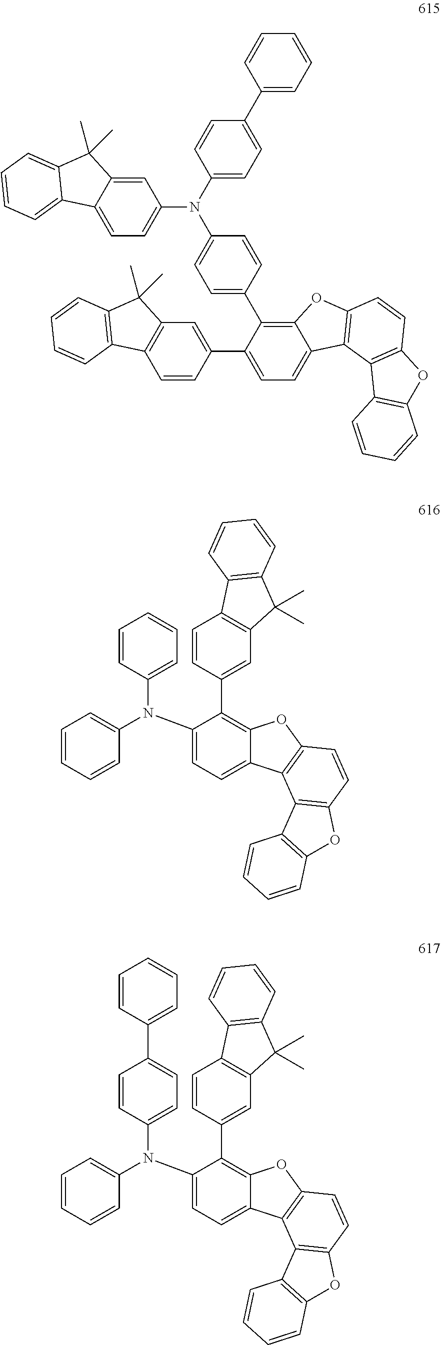 [SMILES: CC1(C)c2ccccc2-c2ccc(-c3ccc4c(oc5ccc6oc7ccccc7c6c54)c3-c3ccc(N(c4ccc(-c5ccccc5)cc4)c4ccc5c(c4)C(C)(C)c4ccccc4-5)cc3)cc21 ; CC1(C)c2ccccc2-c2ccc(-c3c(N(c4ccccc4)c4ccccc4)ccc4c3oc3ccc5oc6ccccc6c5c34)cc21 ; CC1(C)c2ccccc2-c2ccc(-c3c(N(c4ccccc4)c4ccc(-c5ccccc5)cc4)ccc4c3oc3ccc5oc6ccccc6c5c34)cc21]

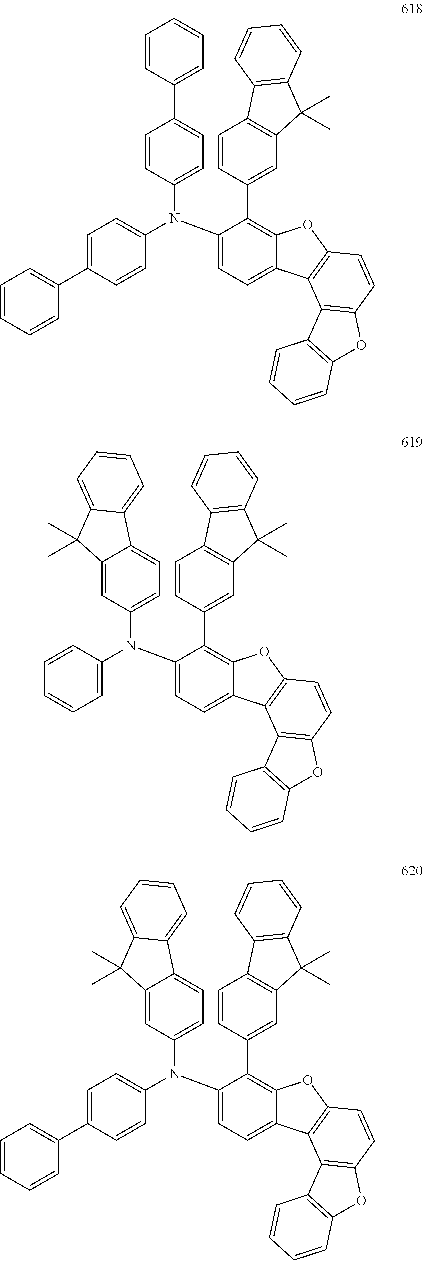 [SMILES: CC1(C)c2ccccc2-c2ccc(-c3c(N(c4ccc(-c5ccccc5)cc4)c4ccc(-c5ccccc5)cc4)ccc4c3oc3ccc5oc6ccccc6c5c34)cc21 ; CC1(C)c2ccccc2-c2ccc(-c3c(N(c4ccccc4)c4ccc5c(c4)C(C)(C)c4ccccc4-5)ccc4c3oc3ccc5oc6ccccc6c5c34)cc21 ; CC1(C)c2ccccc2-c2ccc(-c3c(N(c4ccc(-c5ccccc5)cc4)c4ccc5c(c4)C(C)(C)c4ccccc4-5)ccc4c3oc3ccc5oc6ccccc6c5c34)cc21]

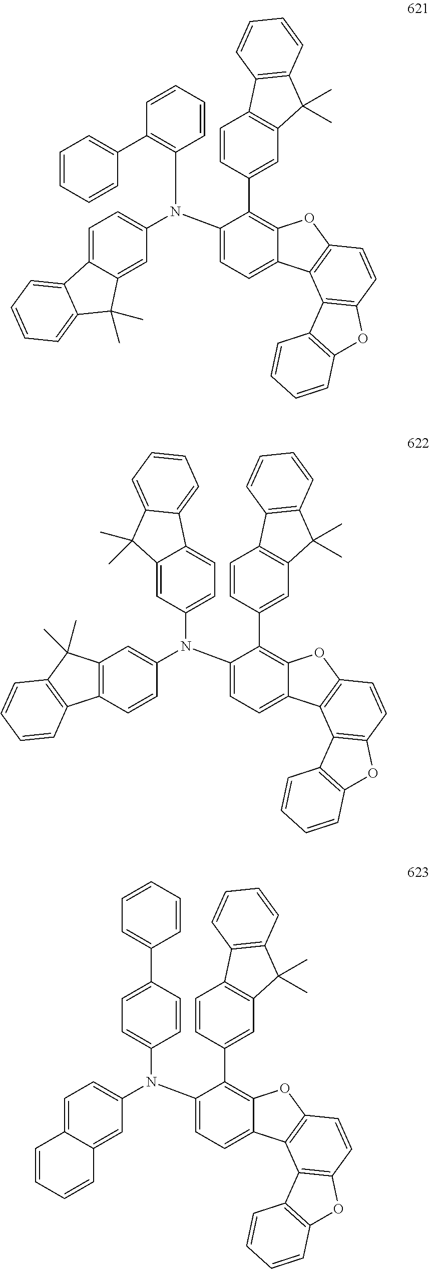 [SMILES: CC1(C)c2ccccc2-c2ccc(-c3c(N(c4ccc5c(c4)C(C)(C)c4ccccc4-5)c4ccccc4-c4ccccc4)ccc4c3oc3ccc5oc6ccccc6c5c34)cc21 ; CC1(C)c2ccccc2-c2ccc(-c3c(N(c4ccc5c(c4)C(C)(C)c4ccccc4-5)c4ccc5c(c4)C(C)(C)c4ccccc4-5)ccc4c3oc3ccc5oc6ccccc6c5c34)cc21 ; CC1(C)c2ccccc2-c2ccc(-c3c(N(c4ccc(-c5ccccc5)cc4)c4ccc5ccccc5c4)ccc4c3oc3ccc5oc6ccccc6c5c34)cc21]

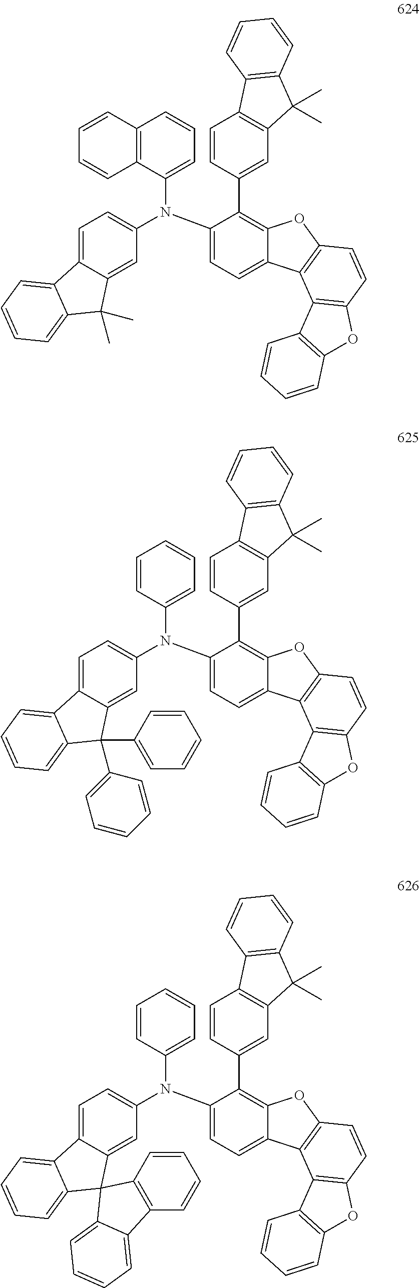 [SMILES: CC1(C)c2ccccc2-c2ccc(-c3c(N(c4ccc5c(c4)C(C)(C)c4ccccc4-5)c4cccc5ccccc45)ccc4c3oc3ccc5oc6ccccc6c5c34)cc21 ; CC1(C)c2ccccc2-c2ccc(-c3c(N(c4ccccc4)c4ccc5c(c4)C(c4ccccc4)(c4ccccc4)c4ccccc4-5)ccc4c3oc3ccc5oc6ccccc6c5c34)cc21 ; CC1(C)c2ccccc2-c2ccc(-c3c(N(c4ccccc4)c4ccc5c(c4)C4(c6ccccc6-c6ccccc64)c4ccccc4-5)ccc4c3oc3ccc5oc6ccccc6c5c34)cc21]

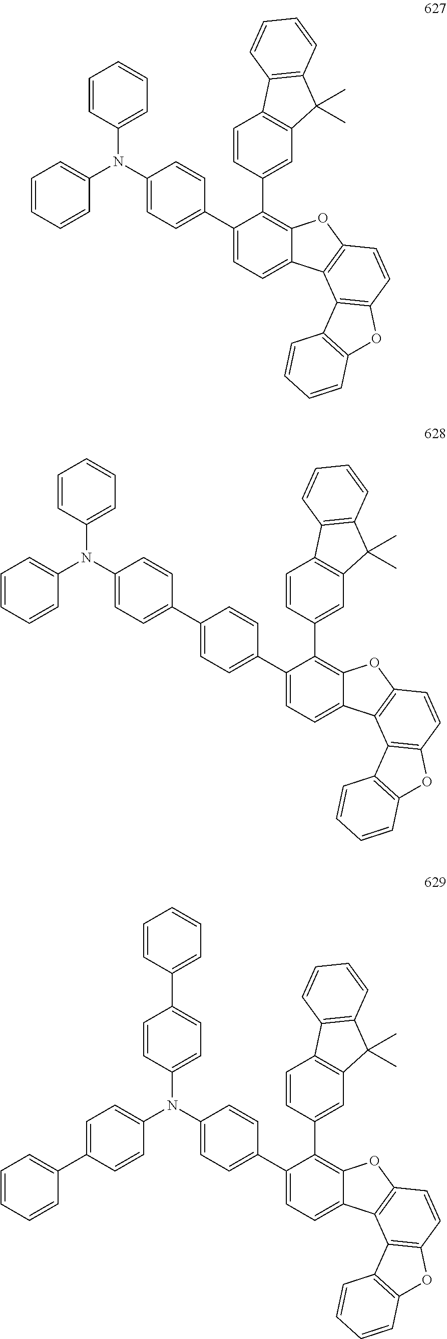 [SMILES: CC1(C)c2ccccc2-c2ccc(-c3c(-c4ccc(N(c5ccccc5)c5ccccc5)cc4)ccc4c3oc3ccc5oc6ccccc6c5c34)cc21 ; CC1(C)c2ccccc2-c2ccc(-c3c(-c4ccc(-c5ccc(N(c6ccccc6)c6ccccc6)cc5)cc4)ccc4c3oc3ccc5oc6ccccc6c5c34)cc21 ; CC1(C)c2ccccc2-c2ccc(-c3c(-c4ccc(N(c5ccc(-c6ccccc6)cc5)c5ccc(-c6ccccc6)cc5)cc4)ccc4c3oc3ccc5oc6ccccc6c5c34)cc21]

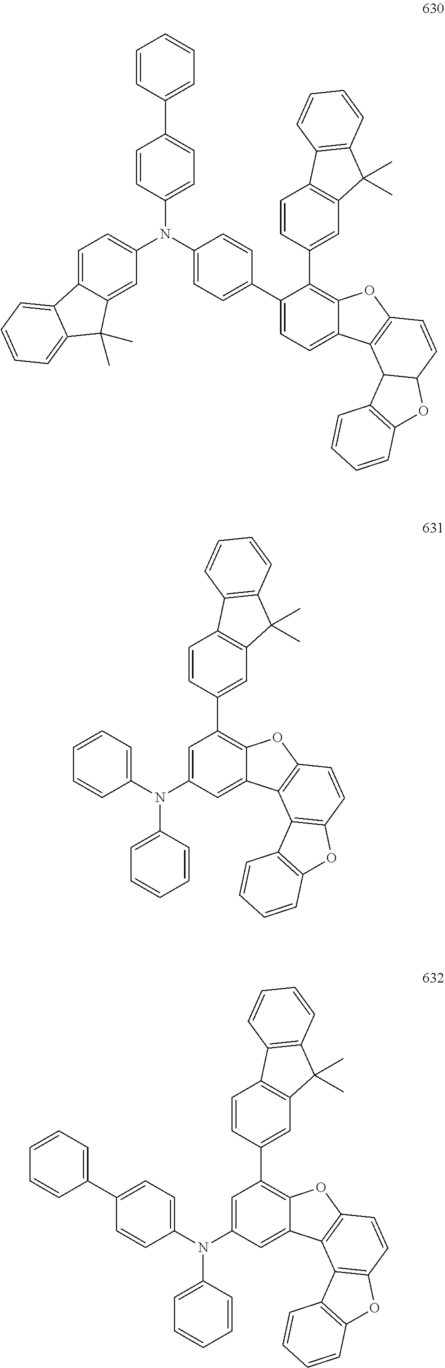 [SMILES: CC1(C)c2ccccc2-c2ccc(-c3c(-c4ccc(N(c5ccc(-c6ccccc6)cc5)c5ccc6c(c5)C(C)(C)c5ccccc5-6)cc4)ccc4c5c(oc34)C=CC3Oc4ccccc4C53)cc21 ; CC1(C)c2ccccc2-c2ccc(-c3cc(N(c4ccccc4)c4ccccc4)cc4c3oc3ccc5oc6ccccc6c5c34)cc21 ; CC1(C)c2ccccc2-c2ccc(-c3cc(N(c4ccccc4)c4ccc(-c5ccccc5)cc4)cc4c3oc3ccc5oc6ccccc6c5c34)cc21]

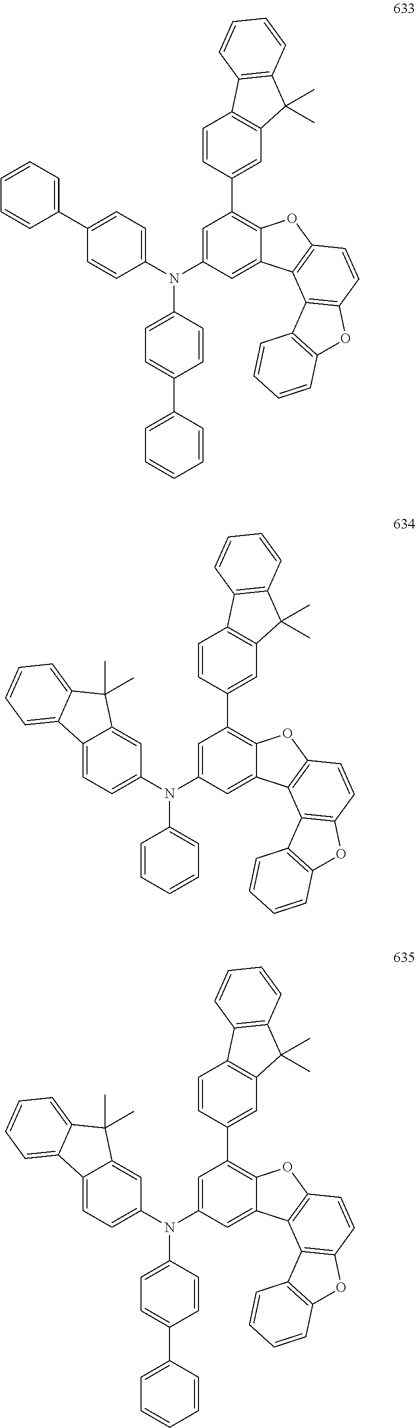 [SMILES: CC1(C)c2ccccc2-c2ccc(-c3cc(N(c4ccc(-c5ccccc5)cc4)c4ccc(-c5ccccc5)cc4)cc4c3oc3ccc5oc6ccccc6c5c34)cc21 ; CC1(C)c2ccccc2-c2ccc(-c3cc(N(c4ccccc4)c4ccc5c(c4)C(C)(C)c4ccccc4-5)cc4c3oc3ccc5oc6ccccc6c5c34)cc21 ; CC1(C)c2ccccc2-c2ccc(-c3cc(N(c4ccc(-c5ccccc5)cc4)c4ccc5c(c4)C(C)(C)c4ccccc4-5)cc4c3oc3ccc5oc6ccccc6c5c34)cc21]

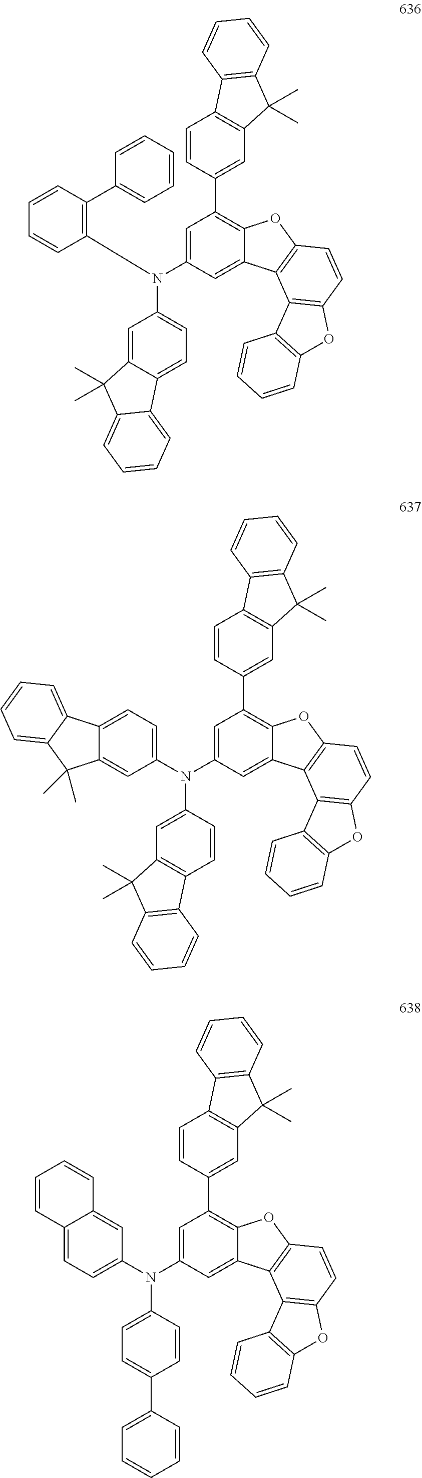 [SMILES: CC1(C)c2ccccc2-c2ccc(-c3cc(N(c4ccc5c(c4)C(C)(C)c4ccccc4-5)c4ccccc4-c4ccccc4)cc4c3oc3ccc5oc6ccccc6c5c34)cc21 ; CC1(C)c2ccccc2-c2ccc(-c3cc(N(c4ccc5c(c4)C(C)(C)c4ccccc4-5)c4ccc5c(c4)C(C)(C)c4ccccc4-5)cc4c3oc3ccc5oc6ccccc6c5c34)cc21 ; CC1(C)c2ccccc2-c2ccc(-c3cc(N(c4ccc(-c5ccccc5)cc4)c4ccc5ccccc5c4)cc4c3oc3ccc5oc6ccccc6c5c34)cc21]

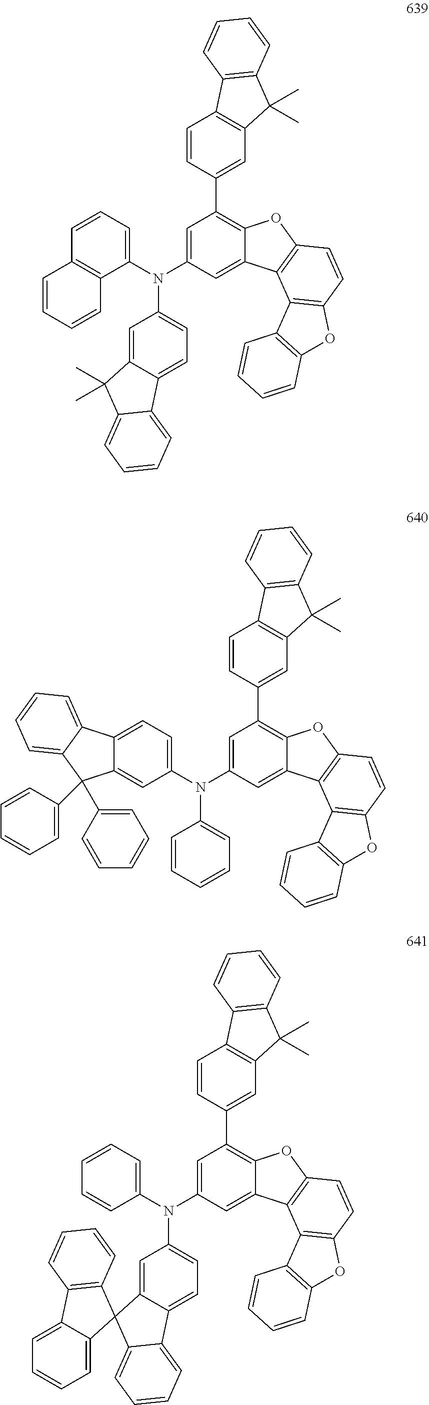 [SMILES: CC1(C)c2ccccc2-c2ccc(-c3cc(N(c4ccc5c(c4)C(C)(C)c4ccccc4-5)c4cccc5ccccc45)cc4c3oc3ccc5oc6ccccc6c5c34)cc21 ; CC1(C)c2ccccc2-c2ccc(-c3cc(N(c4ccccc4)c4ccc5c(c4)C(c4ccccc4)(c4ccccc4)c4ccccc4-5)cc4c3oc3ccc5oc6ccccc6c5c34)cc21 ; CC1(C)c2ccccc2-c2ccc(-c3cc(N(c4ccccc4)c4ccc5c(c4)C4(c6ccccc6-c6ccccc64)c4ccccc4-5)cc4c3oc3ccc5oc6ccccc6c5c34)cc21]

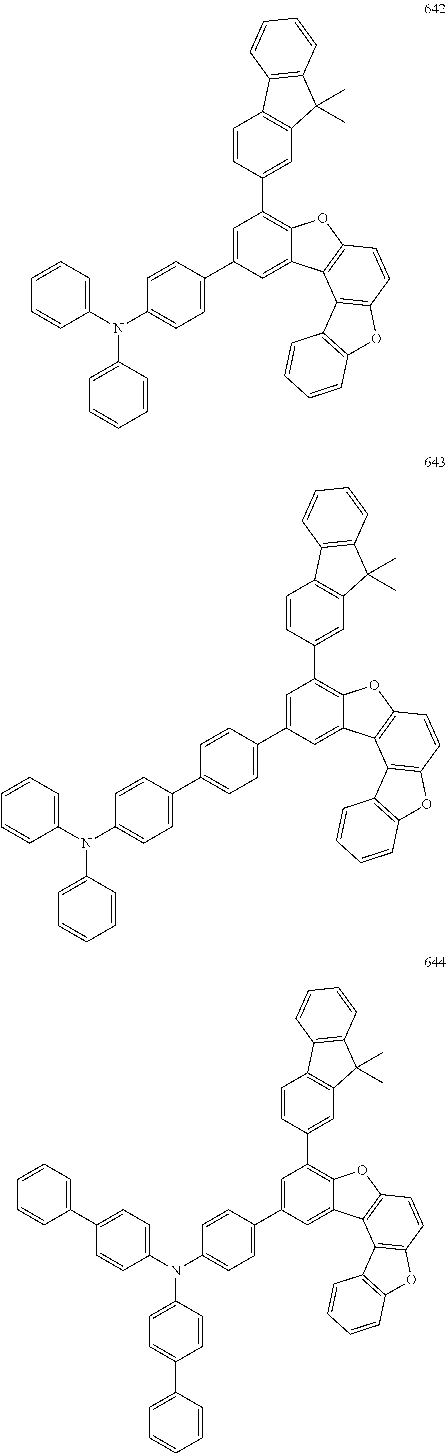 [SMILES: CC1(C)c2ccccc2-c2ccc(-c3cc(-c4ccc(N(c5ccccc5)c5ccccc5)cc4)cc4c3oc3ccc5oc6ccccc6c5c34)cc21 ; CC1(C)c2ccccc2-c2ccc(-c3cc(-c4ccc(-c5ccc(N(c6ccccc6)c6ccccc6)cc5)cc4)cc4c3oc3ccc5oc6ccccc6c5c34)cc21 ; CC1(C)c2ccccc2-c2ccc(-c3cc(-c4ccc(N(c5ccc(-c6ccccc6)cc5)c5ccc(-c6ccccc6)cc5)cc4)cc4c3oc3ccc5oc6ccccc6c5c34)cc21]

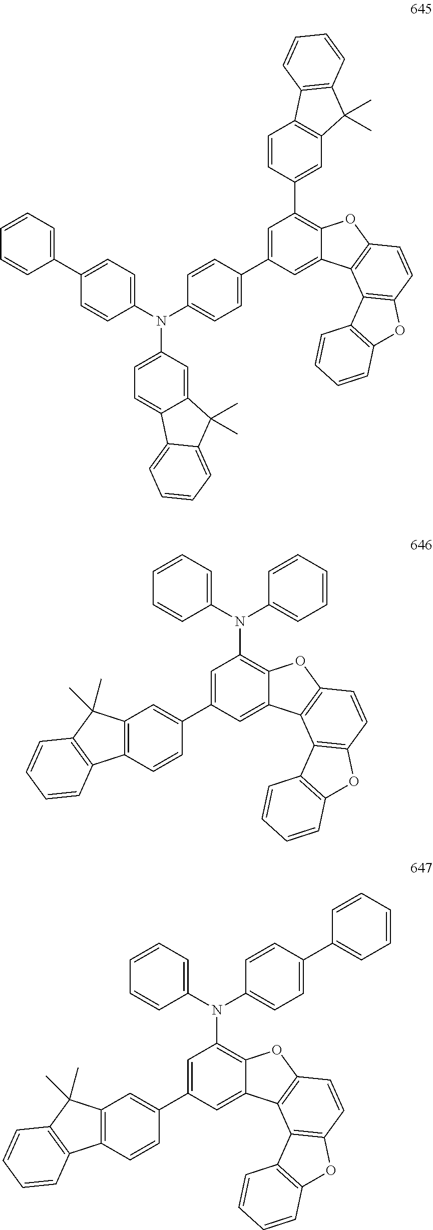 [SMILES: CC1(C)c2ccccc2-c2ccc(-c3cc(-c4ccc(N(c5ccc(-c6ccccc6)cc5)c5ccc6c(c5)C(C)(C)c5ccccc5-6)cc4)cc4c3oc3ccc5oc6ccccc6c5c34)cc21 ; CC1(C)c2ccccc2-c2ccc(-c3cc(N(c4ccccc4)c4ccccc4)c4oc5ccc6oc7ccccc7c6c5c4c3)cc21 ; CC1(C)c2ccccc2-c2ccc(-c3cc(N(c4ccccc4)c4ccc(-c5ccccc5)cc4)c4oc5ccc6oc7ccccc7c6c5c4c3)cc21]

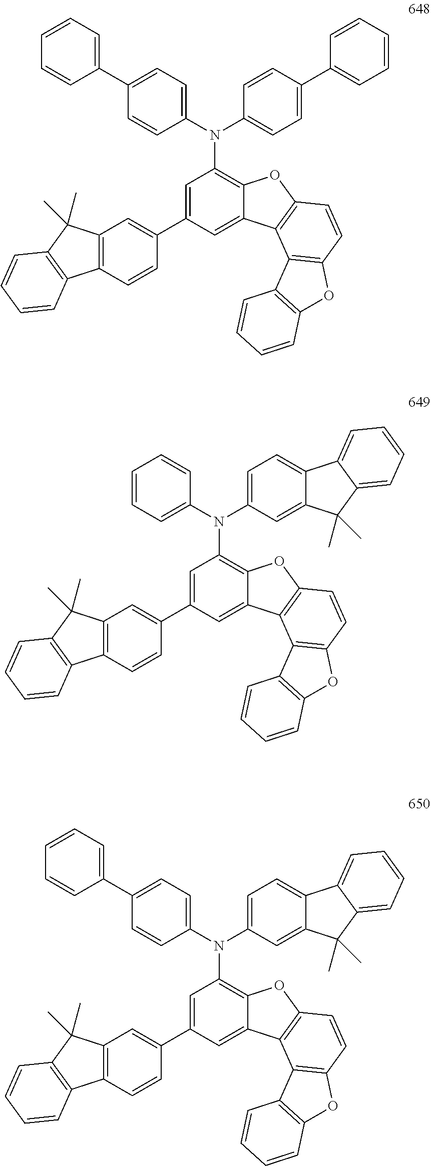 [SMILES: CC1(C)c2ccccc2-c2ccc(-c3cc(N(c4ccc(-c5ccccc5)cc4)c4ccc(-c5ccccc5)cc4)c4oc5ccc6oc7ccccc7c6c5c4c3)cc21 ; CC1(C)c2ccccc2-c2ccc(-c3cc(N(c4ccccc4)c4ccc5c(c4)C(C)(C)c4ccccc4-5)c4oc5ccc6oc7ccccc7c6c5c4c3)cc21 ; CC1(C)c2ccccc2-c2ccc(-c3cc(N(c4ccc(-c5ccccc5)cc4)c4ccc5c(c4)C(C)(C)c4ccccc4-5)c4oc5ccc6oc7ccccc7c6c5c4c3)cc21]

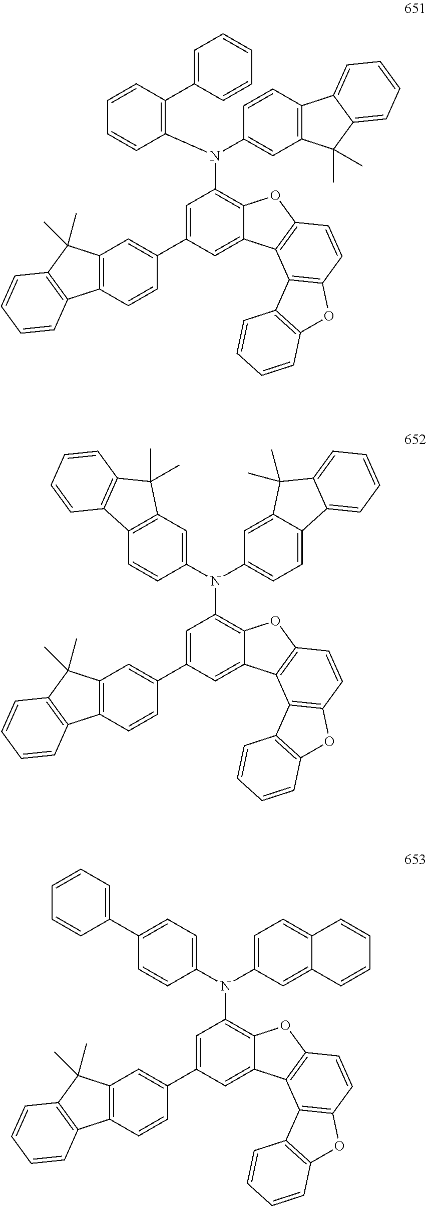 [SMILES: CC1(C)c2ccccc2-c2ccc(-c3cc(N(c4ccc5c(c4)C(C)(C)c4ccccc4-5)c4ccccc4-c4ccccc4)c4oc5ccc6oc7ccccc7c6c5c4c3)cc21 ; CC1(C)c2ccccc2-c2ccc(-c3cc(N(c4ccc5c(c4)C(C)(C)c4ccccc4-5)c4ccc5c(c4)C(C)(C)c4ccccc4-5)c4oc5ccc6oc7ccccc7c6c5c4c3)cc21 ; CC1(C)c2ccccc2-c2ccc(-c3cc(N(c4ccc(-c5ccccc5)cc4)c4ccc5ccccc5c4)c4oc5ccc6oc7ccccc7c6c5c4c3)cc21]

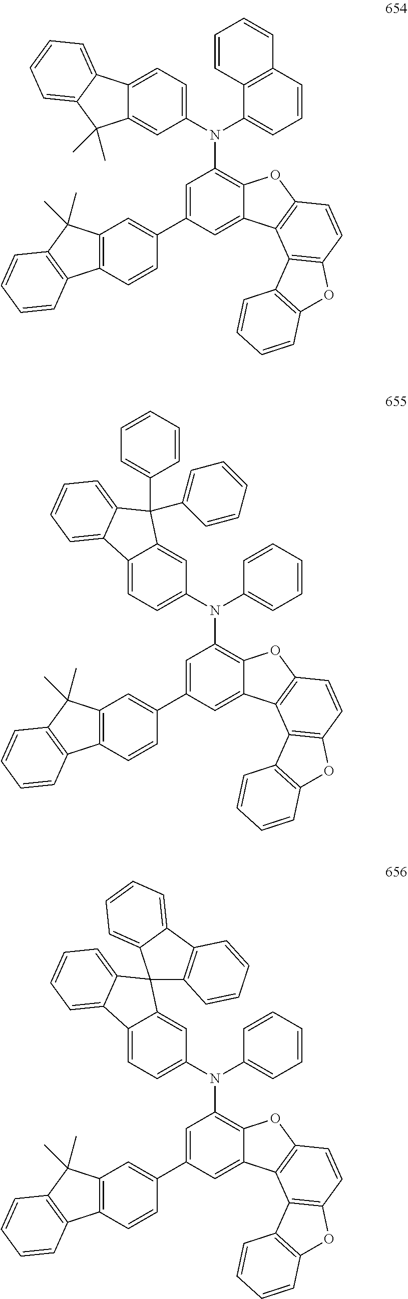 [SMILES: CC1(C)c2ccccc2-c2ccc(-c3cc(N(c4ccc5c(c4)C(C)(C)c4ccccc4-5)c4cccc5ccccc45)c4oc5ccc6oc7ccccc7c6c5c4c3)cc21 ; CC1(C)c2ccccc2-c2ccc(-c3cc(N(c4ccccc4)c4ccc5c(c4)C(c4ccccc4)(c4ccccc4)c4ccccc4-5)c4oc5ccc6oc7ccccc7c6c5c4c3)cc21 ; CC1(C)c2ccccc2-c2ccc(-c3cc(N(c4ccccc4)c4ccc5c(c4)C4(c6ccccc6-c6ccccc64)c4ccccc4-5)c4oc5ccc6oc7ccccc7c6c5c4c3)cc21]

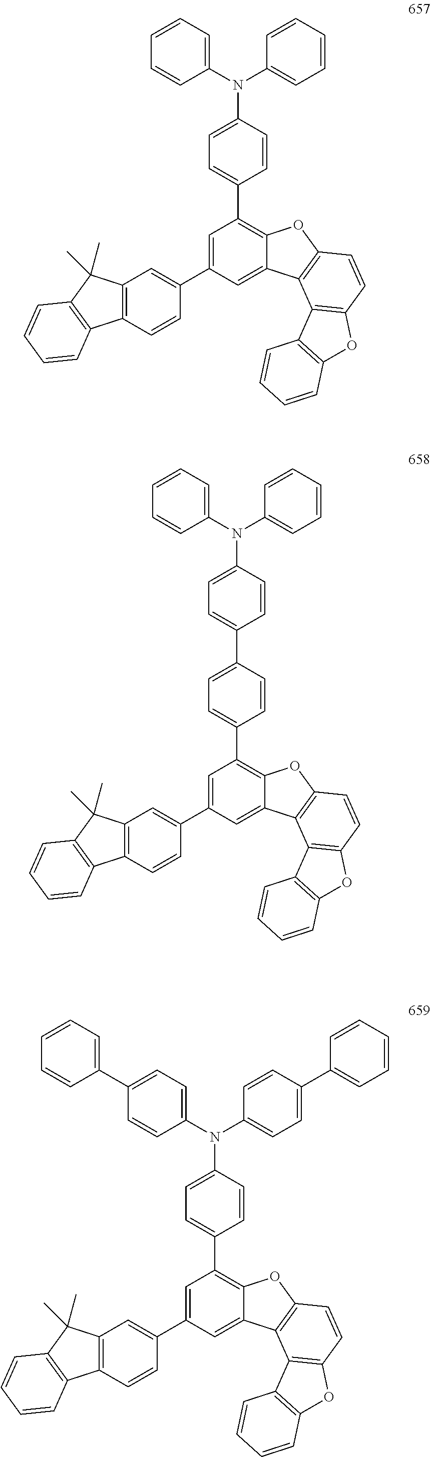 [SMILES: CC1(C)c2ccccc2-c2ccc(-c3cc(-c4ccc(N(c5ccccc5)c5ccccc5)cc4)c4oc5ccc6oc7ccccc7c6c5c4c3)cc21 ; CC1(C)c2ccccc2-c2ccc(-c3cc(-c4ccc(-c5ccc(N(c6ccccc6)c6ccccc6)cc5)cc4)c4oc5ccc6oc7ccccc7c6c5c4c3)cc21 ; CC1(C)c2ccccc2-c2ccc(-c3cc(-c4ccc(N(c5ccc(-c6ccccc6)cc5)c5ccc(-c6ccccc6)cc5)cc4)c4oc5ccc6oc7ccccc7c6c5c4c3)cc21]

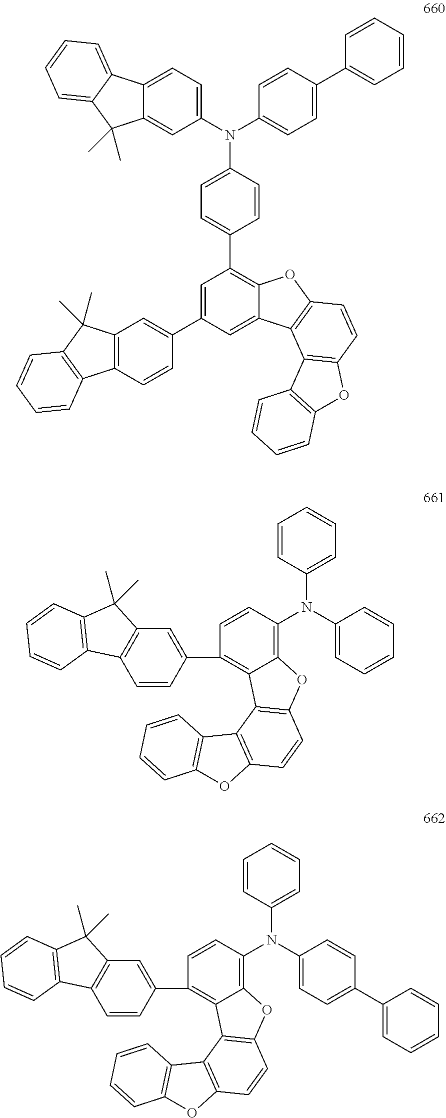 [SMILES: CC1(C)c2ccccc2-c2ccc(-c3cc(-c4ccc(N(c5ccc(-c6ccccc6)cc5)c5ccc6c(c5)C(C)(C)c5ccccc5-6)cc4)c4oc5ccc6oc7ccccc7c6c5c4c3)cc21 ; CC1(C)c2ccccc2-c2ccc(-c3ccc(N(c4ccccc4)c4ccccc4)c4oc5ccc6oc7ccccc7c6c5c34)cc21 ; CC1(C)c2ccccc2-c2ccc(-c3ccc(N(c4ccccc4)c4ccc(-c5ccccc5)cc4)c4oc5ccc6oc7ccccc7c6c5c34)cc21]

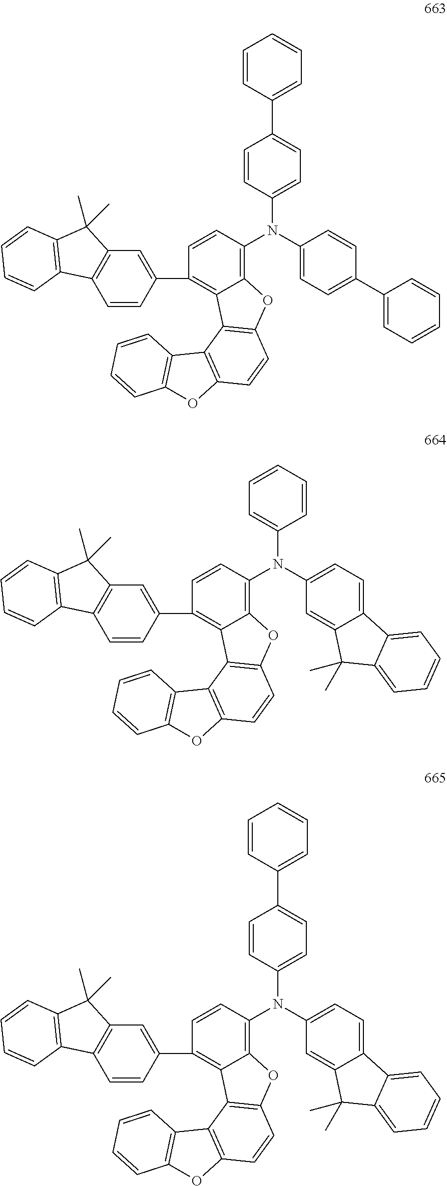 [SMILES: CC1(C)c2ccccc2-c2ccc(-c3ccc(N(c4ccc(-c5ccccc5)cc4)c4ccc(-c5ccccc5)cc4)c4oc5ccc6oc7ccccc7c6c5c34)cc21 ; CC1(C)c2ccccc2-c2ccc(-c3ccc(N(c4ccccc4)c4ccc5c(c4)C(C)(C)c4ccccc4-5)c4oc5ccc6oc7ccccc7c6c5c34)cc21 ; CC1(C)c2ccccc2-c2ccc(-c3ccc(N(c4ccc(-c5ccccc5)cc4)c4ccc5c(c4)C(C)(C)c4ccccc4-5)c4oc5ccc6oc7ccccc7c6c5c34)cc21]

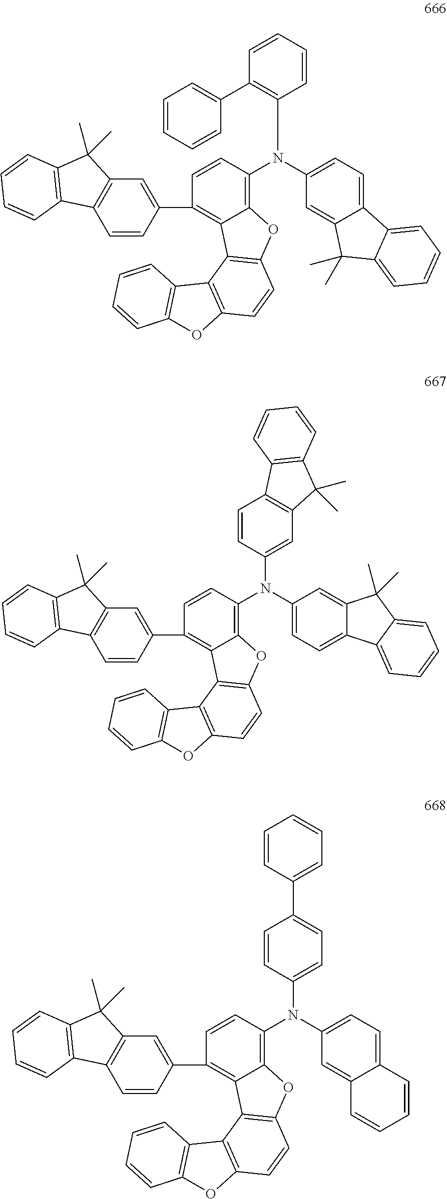 [SMILES: CC1(C)c2ccccc2-c2ccc(-c3ccc(N(c4ccc5c(c4)C(C)(C)c4ccccc4-5)c4ccccc4-c4ccccc4)c4oc5ccc6oc7ccccc7c6c5c34)cc21 ; CC1(C)c2ccccc2-c2ccc(-c3ccc(N(c4ccc5c(c4)C(C)(C)c4ccccc4-5)c4ccc5c(c4)C(C)(C)c4ccccc4-5)c4oc5ccc6oc7ccccc7c6c5c34)cc21 ; CC1(C)c2ccccc2-c2ccc(-c3ccc(N(c4ccc(-c5ccccc5)cc4)c4ccc5ccccc5c4)c4oc5ccc6oc7ccccc7c6c5c34)cc21]

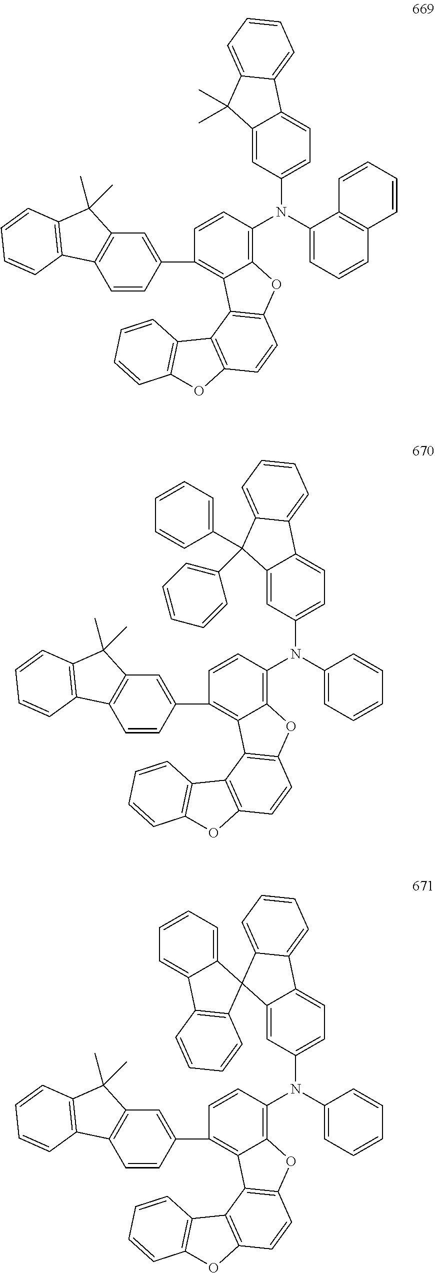 [SMILES: CC1(C)c2ccccc2-c2ccc(-c3ccc(N(c4ccc5c(c4)C(C)(C)c4ccccc4-5)c4cccc5ccccc45)c4oc5ccc6oc7ccccc7c6c5c34)cc21 ; CC1(C)c2ccccc2-c2ccc(-c3ccc(N(c4ccccc4)c4ccc5c(c4)C(c4ccccc4)(c4ccccc4)c4ccccc4-5)c4oc5ccc6oc7ccccc7c6c5c34)cc21 ; CC1(C)c2ccccc2-c2ccc(-c3ccc(N(c4ccccc4)c4ccc5c(c4)C4(c6ccccc6-c6ccccc64)c4ccccc4-5)c4oc5ccc6oc7ccccc7c6c5c34)cc21]

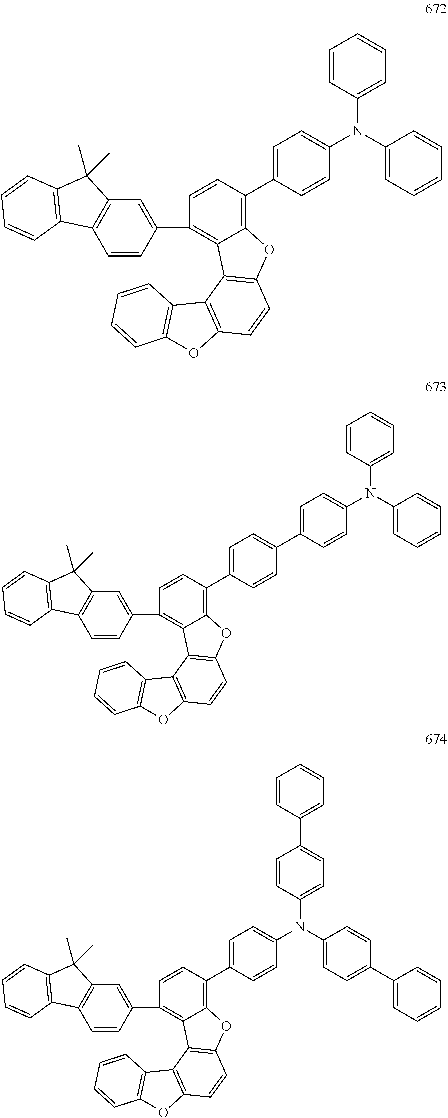 [SMILES: CC1(C)c2ccccc2-c2ccc(-c3ccc(-c4ccc(N(c5ccccc5)c5ccccc5)cc4)c4oc5ccc6oc7ccccc7c6c5c34)cc21 ; CC1(C)c2ccccc2-c2ccc(-c3ccc(-c4ccc(-c5ccc(N(c6ccccc6)c6ccccc6)cc5)cc4)c4oc5ccc6oc7ccccc7c6c5c34)cc21 ; CC1(C)c2ccccc2-c2ccc(-c3ccc(-c4ccc(N(c5ccc(-c6ccccc6)cc5)c5ccc(-c6ccccc6)cc5)cc4)c4oc5ccc6oc7ccccc7c6c5c34)cc21]

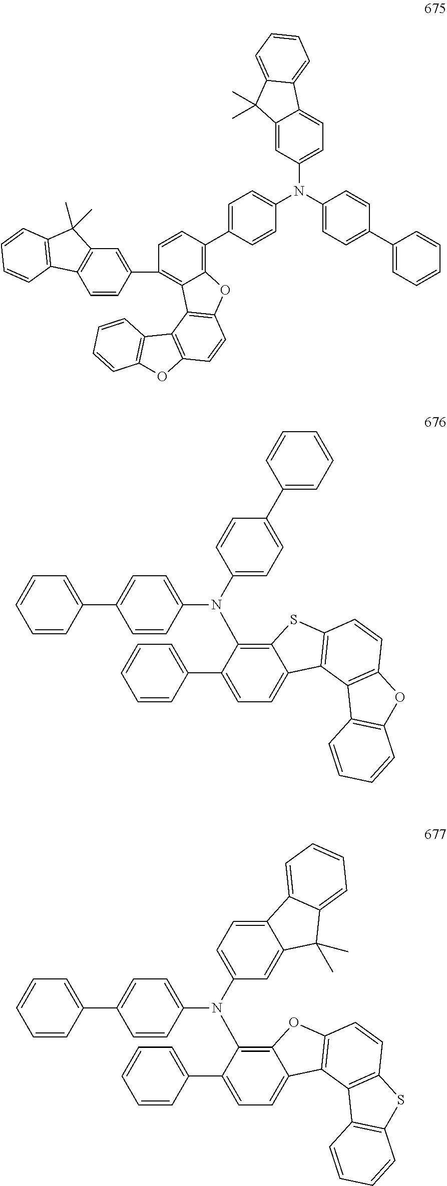 [SMILES: CC1(C)c2ccccc2-c2ccc(-c3ccc(-c4ccc(N(c5ccc(-c6ccccc6)cc5)c5ccc6c(c5)C(C)(C)c5ccccc5-6)cc4)c4oc5ccc6oc7ccccc7c6c5c34)cc21 ; c1ccc(-c2ccc(N(c3ccc(-c4ccccc4)cc3)c3c(-c4ccccc4)ccc4c3sc3ccc5oc6ccccc6c5c34)cc2)cc1 ; CC1(C)c2ccccc2-c2ccc(N(c3ccc(-c4ccccc4)cc3)c3c(-c4ccccc4)ccc4c3oc3ccc5sc6ccccc6c5c34)cc21]

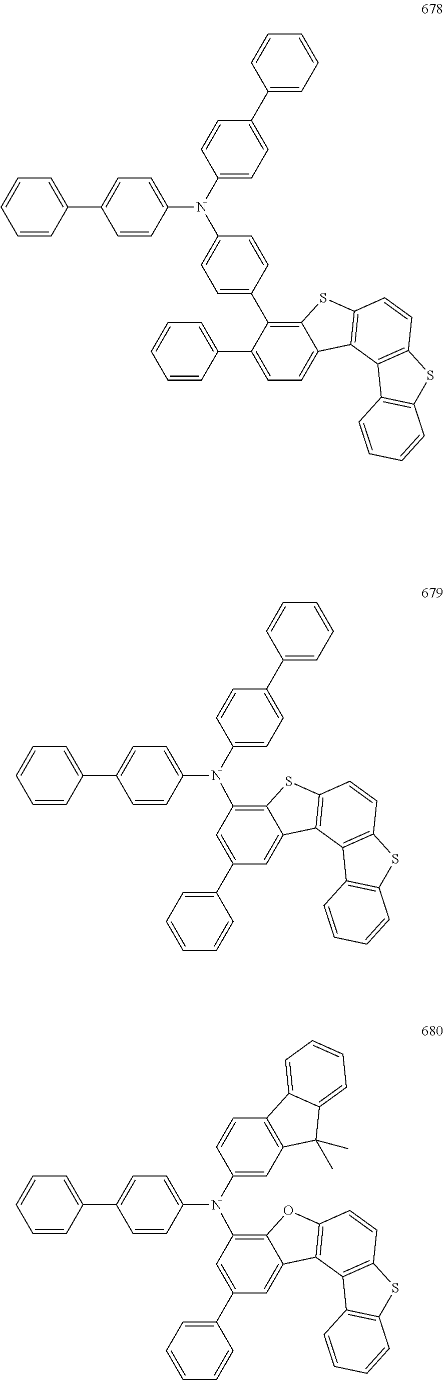 [SMILES: c1ccc(-c2ccc(N(c3ccc(-c4ccccc4)cc3)c3ccc(-c4c(-c5ccccc5)ccc5c4sc4ccc6sc7ccccc7c6c45)cc3)cc2)cc1 ; c1ccc(-c2ccc(N(c3ccc(-c4ccccc4)cc3)c3cc(-c4ccccc4)cc4c3sc3ccc5sc6ccccc6c5c34)cc2)cc1 ; CC1(C)c2ccccc2-c2ccc(N(c3ccc(-c4ccccc4)cc3)c3cc(-c4ccccc4)cc4c3oc3ccc5sc6ccccc6c5c34)cc21]

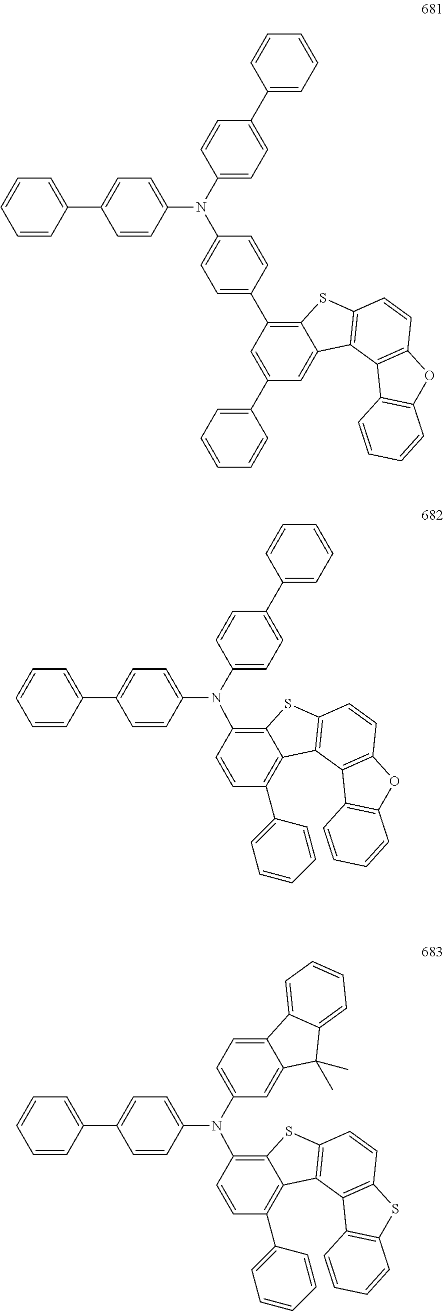 [SMILES: c1ccc(-c2ccc(N(c3ccc(-c4ccccc4)cc3)c3ccc(-c4cc(-c5ccccc5)cc5c4sc4ccc6oc7ccccc7c6c45)cc3)cc2)cc1 ; c1ccc(-c2ccc(N(c3ccc(-c4ccccc4)cc3)c3ccc(-c4ccccc4)c4c3sc3ccc5oc6ccccc6c5c34)cc2)cc1 ; CC1(C)c2ccccc2-c2ccc(N(c3ccc(-c4ccccc4)cc3)c3ccc(-c4ccccc4)c4c3sc3ccc5sc6ccccc6c5c34)cc21]

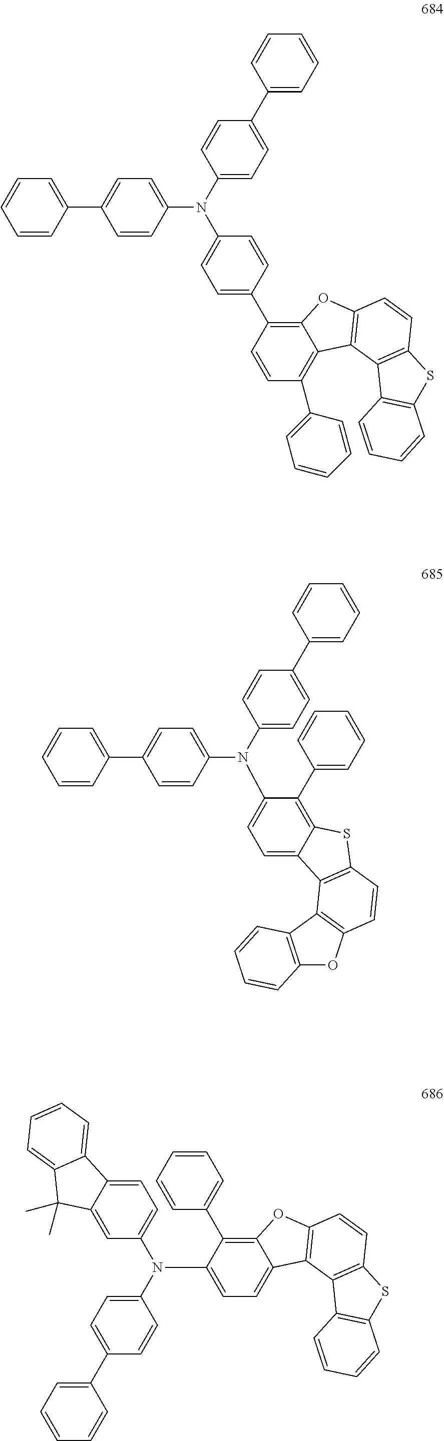 [SMILES: c1ccc(-c2ccc(N(c3ccc(-c4ccccc4)cc3)c3ccc(-c4ccc(-c5ccccc5)c5c4oc4ccc6sc7ccccc7c6c45)cc3)cc2)cc1 ; c1ccc(-c2ccc(N(c3ccc(-c4ccccc4)cc3)c3ccc4c(sc5ccc6oc7ccccc7c6c54)c3-c3ccccc3)cc2)cc1 ; CC1(C)c2ccccc2-c2ccc(N(c3ccc(-c4ccccc4)cc3)c3ccc4c(oc5ccc6sc7ccccc7c6c54)c3-c3ccccc3)cc21]

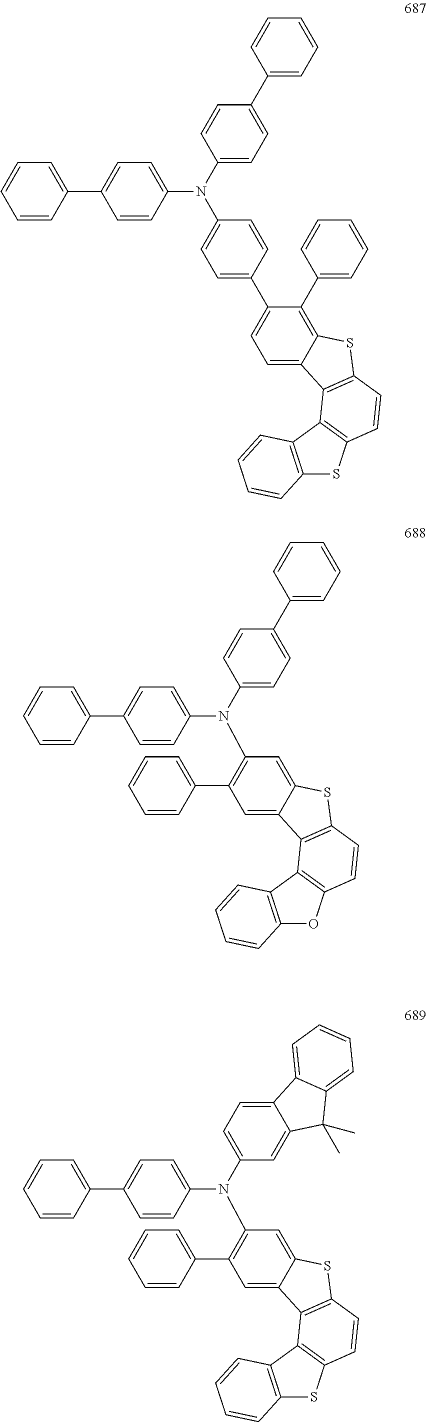

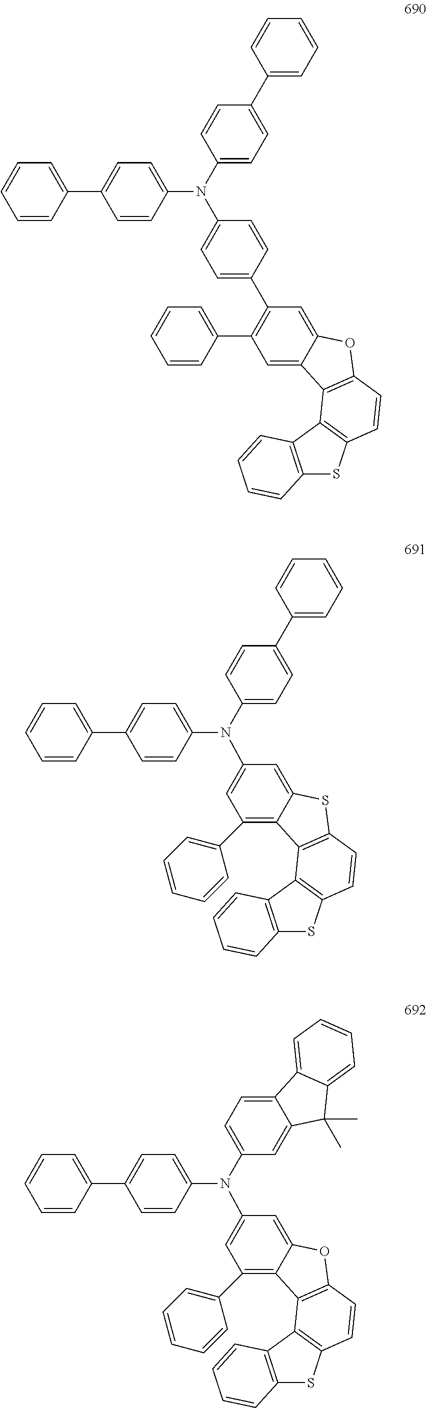 [SMILES: c1ccc(-c2ccc(N(c3ccc(-c4ccccc4)cc3)c3ccc(-c4cc5oc6ccc7sc8ccccc8c7c6c5cc4-c4ccccc4)cc3)cc2)cc1 ; c1ccc(-c2ccc(N(c3ccc(-c4ccccc4)cc3)c3cc(-c4ccccc4)c4c(c3)sc3ccc5sc6ccccc6c5c34)cc2)cc1 ; CC1(C)c2ccccc2-c2ccc(N(c3ccc(-c4ccccc4)cc3)c3cc(-c4ccccc4)c4c(c3)oc3ccc5sc6ccccc6c5c34)cc21]

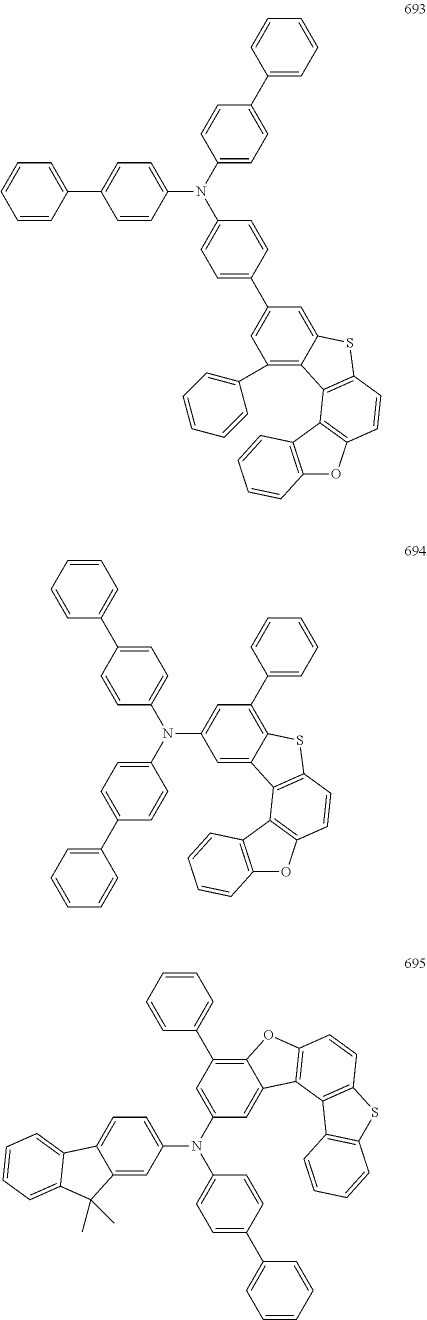 [SMILES: c1ccc(-c2ccc(N(c3ccc(-c4ccccc4)cc3)c3ccc(-c4cc(-c5ccccc5)c5c(c4)sc4ccc6oc7ccccc7c6c45)cc3)cc2)cc1 ; c1ccc(-c2ccc(N(c3ccc(-c4ccccc4)cc3)c3cc(-c4ccccc4)c4sc5ccc6oc7ccccc7c6c5c4c3)cc2)cc1 ; CC1(C)c2ccccc2-c2ccc(N(c3ccc(-c4ccccc4)cc3)c3cc(-c4ccccc4)c4oc5ccc6sc7ccccc7c6c5c4c3)cc21]

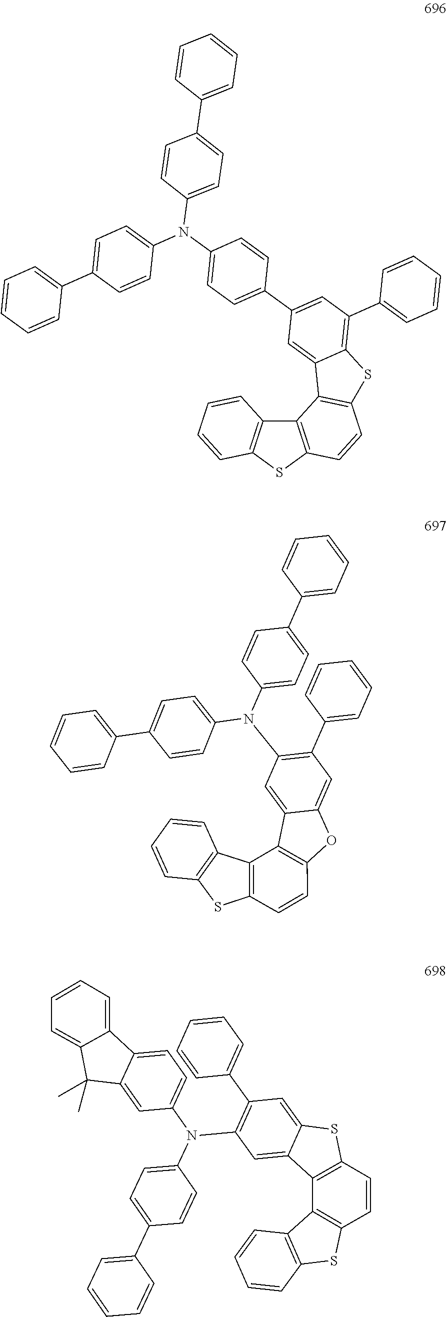 [SMILES: c1ccc(-c2ccc(N(c3ccc(-c4ccccc4)cc3)c3ccc(-c4cc(-c5ccccc5)c5sc6ccc7sc8ccccc8c7c6c5c4)cc3)cc2)cc1 ; c1ccc(-c2ccc(N(c3ccc(-c4ccccc4)cc3)c3cc4c(cc3-c3ccccc3)oc3ccc5sc6ccccc6c5c34)cc2)cc1 ; CC1(C)c2ccccc2-c2ccc(N(c3ccc(-c4ccccc4)cc3)c3cc4c(cc3-c3ccccc3)sc3ccc5sc6ccccc6c5c34)cc21]

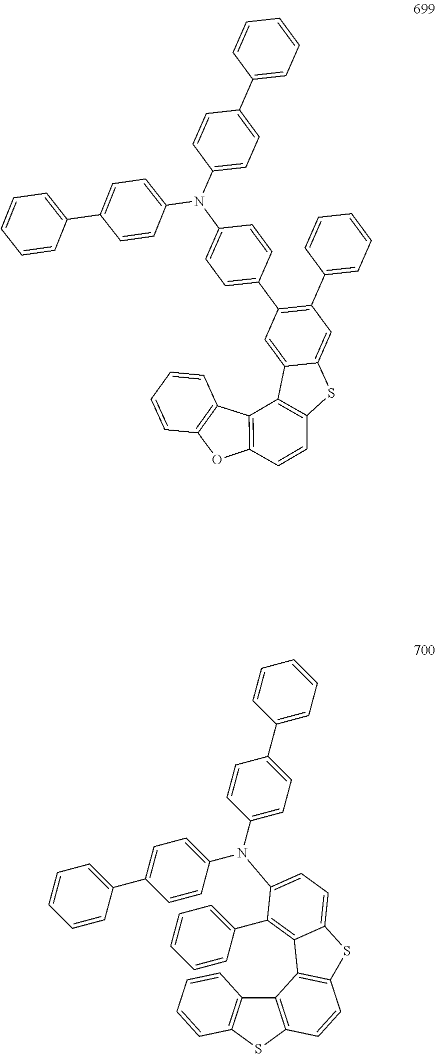 [SMILES: c1ccc(-c2ccc(N(c3ccc(-c4ccccc4)cc3)c3ccc(-c4cc5c(cc4-c4ccccc4)sc4ccc6oc7ccccc7c6c45)cc3)cc2)cc1 ; c1ccc(-c2ccc(N(c3ccc(-c4ccccc4)cc3)c3ccc4sc5ccc6sc7ccccc7c6c5c4c3-c3ccccc3)cc2)cc1]

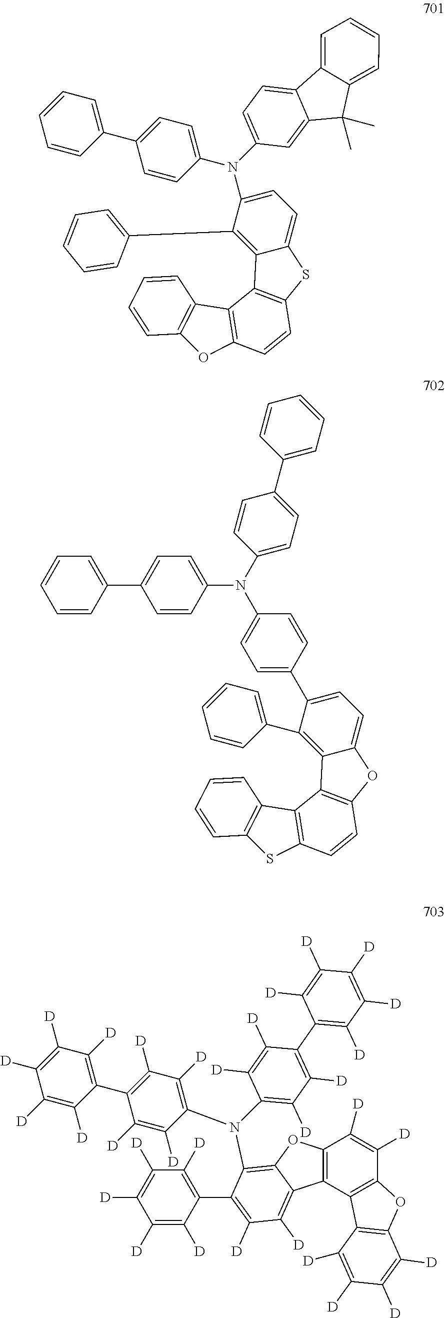 [SMILES: CC1(C)c2ccccc2-c2ccc(N(c3ccc(-c4ccccc4)cc3)c3ccc4sc5ccc6oc7ccccc7c6c5c4c3-c3ccccc3)cc21 ; c1ccc(-c2ccc(N(c3ccc(-c4ccccc4)cc3)c3ccc(-c4ccc5oc6ccc7sc8ccccc8c7c6c5c4-c4ccccc4)cc3)cc2)cc1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c(-c4c([2H])c([2H])c([2H])c([2H])c4[2H])c([2H])c([2H])c4c3oc3c([2H])c([2H])c5oc6c([2H])c([2H])c([2H])c([2H])c6c5c34)c([2H])c2[2H])c([2H])c1[2H]]

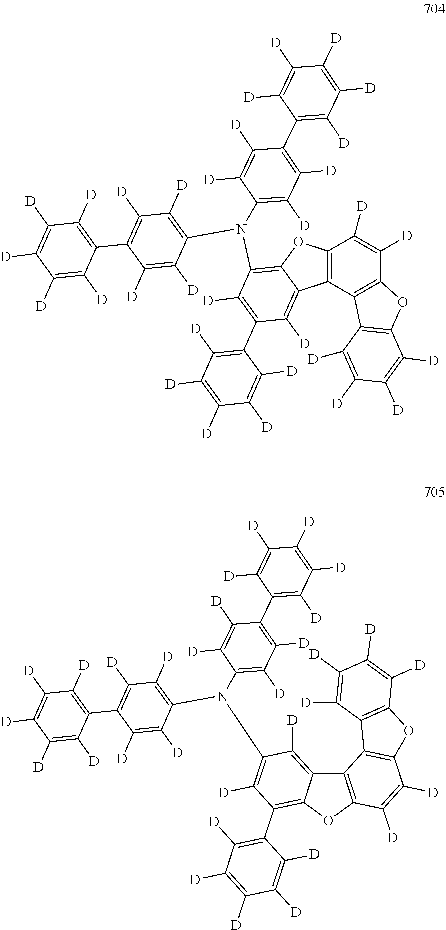

The compound of Formula 1 may be synthesized as a compound having intrinsic properties of the introduced substituent by introducing various substituents into the corresponding structure. For example, by introducing a substituent mainly used for a hole injection layer material, a hole transport layer material, an electron-blocking layer material, a light-emitting layer material, a hole-blocking layer material, an electron transport layer material, and an electron injection layer material used in manufacturing the organic light-emitting device into the core structure, it is possible to synthesize a material satisfying the conditions required for each organic layer.

In addition, by introducing various substituents into the structure of the compound of Formula 1, it is possible to finely control the energy band gap, while by improving the properties at the interface between organic materials, it is possible to diversify the use of the material.

The heterocyclic compound may be used as one or more uses selected from a hole injection layer material, a hole transport layer material, an electron-blocking layer material, a light-emitting layer material, a hole-blocking layer material, an electron transport layer material, and an electron injection layer material, which are used in the organic layer of the organic light-emitting device, and in particular, may be preferably used as a hole transport layer material, an electron-blocking layer material, and a light-emitting layer material.

When the heterocyclic compound is used as a light-emitting layer material, it may be used as a host material, and it may be more usefully used as a p-Host (p-type host) having good hole transport ability among host materials.

In addition, the present invention relates to an light-emitting device comprising:
a first electrode;
a second electrode provided to face the first electrode; and
one or more organic layers provided between the first electrode and the second electrode, and
wherein one or more of the organic layers comprise the heterocyclic compound represented by Formula 1.

In one embodiment of the present invention, the first electrode may be an anode, and the second electrode may be a cathode.

In another embodiment, the first electrode may be a cathode, and the second electrode may be an anode.

The organic light-emitting device according to one embodiment of the present invention may further comprise one or more layers selected from the group consisting of a hole injection layer, a hole transport layer, an electron-blocking layer, a light-emitting layer, a hole-blocking layer, an electron transport layer, and an electron injection layer, and they may have, but are not limited to, a stack structure in the order of anode/hole injection layer/hole transport layer/electron-blocking layer/light-emitting layer/hole-blocking layer/electron transport layer/electron injection layer/cathode.

In one embodiment of the present invention, the organic light-emitting device may be a green organic light-emitting device, and the heterocyclic compound represented by Formula 1 may be used as a material of the green organic light-emitting device.

In one embodiment of the present invention, the organic light-emitting device may be a blue organic light-emitting device, and the heterocyclic compound represented by Formula 1 may be used as a material of the blue organic light-emitting device.

In one embodiment of the present invention, the organic light-emitting device may be a red organic light-emitting device, and the heterocyclic compound represented by Formula 1 may be used as a material of the red organic light-emitting device.

In one embodiment of the present invention,

In the green organic light-emitting device, the blue organic light-emitting device, and the red organic light-emitting device, the heterocyclic compound represented by Formula 1 may be used as a hole injection layer material, a hole transport layer material, an electron-blocking layer material, a light-emitting layer material, a hole-blocking layer material, an electron transport layer material, and an electron injection layer material, and in particular, may be preferably used as a hole transport layer material, an electron-blocking layer material, and a light-emitting layer material.

When it is used as a light-emitting layer material, it may be used as a host material, and it may be more usefully used as a p-Host (p-type host) having good hole transport ability among host materials.

The host material may include the heterocyclic compound represented by Formula 1 alone, or may include the heterocyclic compound in combination with other host materials.

Specific contents of the heterocyclic compound represented by Formula 1 are the same as described above.

The organic light-emitting device of the present invention may be manufactured by conventional methods and materials for manufacturing an organic light-emitting device, except that one or more organic layers are formed using the aforementioned heterocyclic compound.

The heterocyclic compound may form an organic layer by a solution coating method as well as a vacuum deposition method when manufacturing an organic light-emitting device. Wherein the solution coating method refers to, but is not limited to, spin coating, dip coating, inkjet printing, screen printing, spraying, roll coating, and the like.

The organic layer of the organic light-emitting device of the present invention may have a single-layer structure, but may have a multi-layer structure in which two or more organic layers are stacked. For example, the organic light-emitting device of the present invention may have a structure comprising a hole injection layer, a hole transport layer, an electron-blocking layer, light-emitting layer, a hole-blocking layer, an electron transport layer, an electron injection layer, and the like, as an organic layer. However, the structure of the organic light-emitting device is not limited to such a structure, and may include a smaller or larger number of organic layers.

In one embodiment of the present invention, the light-emitting layer included in the organic layer may further include a phosphorescent dopant.

As the phosphorescent dopant material, those known in the art may be used. For example, phosphorescent dopant materials represented by LL′MX′, LL′L″M, LMX′X″, L₂MX′, and L₃M may be used, but the scope of the present invention is not limited by these examples.

Wherein M may be iridium, platinum, osmium, or the like.

Wherein L is an anionic bidentate ligand coordinated to M by sp² carbon and a heteroatom, and X may function to trap electrons or holes. Non-limiting examples of L include 2-(1-naphthyl)benzoxazole, (2-phenylbenzoxazole), (2-phenylbenzothiazole), (7,8-benzoquinoline), (thiophenepyrizine), phenylpyridine, benzothiophenepyrizine, 3-methoxy-2-phenylpyridine, thiophenepyrizine, tolylpyridine, and the like. Non-limiting examples of X′ and X″ include acetylacetonate (acac), hexafluoroacetylacetonate, salicylidene, picolinate, 8-hydroxyquinolinate, and the like.

Specific examples of the phosphorescent dopant are shown below, but are not limited to these examples.

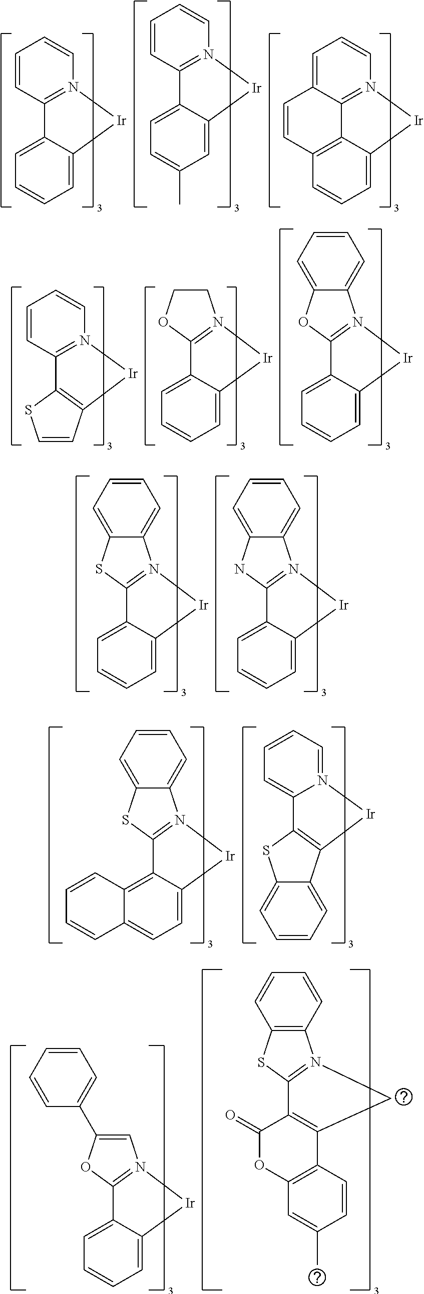

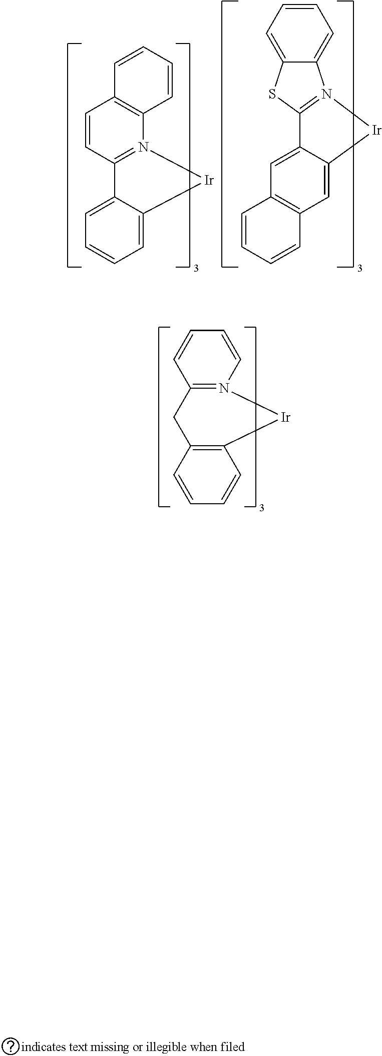

In one embodiment of the present invention, the light-emitting layer includes the heterocyclic compound represented by Formula 1, and may be used together with an iridium-based dopant.

In one embodiment of the present invention, as the iridium-based dopant, the red phosphorescent dopant (piq)₂(Ir) (acac), the green phosphorescent dopant Ir(ppy)₃, and the like may be used.

In one embodiment of the present invention, the dopant may have a content of 1% to 15%, preferably 3% to 10%, more preferably 5% to 10% based on the entire light-emitting layer.

In the organic light-emitting device according to one embodiment of the present invention, materials other than the heterocyclic compound represented by Formula 1 are exemplified below, but these are for illustration only and not for limiting the scope of the present invention, and may be replaced with materials known in the art.

Materials having a relatively large work function may be used as the anode material, and transparent conductive oxides, metals, conductive polymers, or the like may be used. Specific examples of the anode material include, but are not limited to, metals such as vanadium, chromium, copper, zinc, and gold, or alloys thereof; metal oxides such as zinc oxide, indium oxide, indium tin oxide (ITO), and indium zinc oxide (IZO); a combination of metals and oxides such as ZnO: Al or SnO₂: Sb; conductive polymers such as poly(3-methylthiophene), poly[3,4-(ethylene-1,2-dioxy)thiophene] (PEDOT), polypyrrole, and polyaniline; and the like.

Materials having a relatively low work function may be used as the cathode material, and metals, metal oxides, conductive polymers, or the like may be used. Specific examples of the cathode material include, but are not limited to, metals such as magnesium, calcium, sodium, potassium, titanium, indium, yttrium, lithium, gadolinium, aluminum, silver, tin, and lead, or alloys thereof; multilayer structure materials such as LiF/Al or LiO₂/Al.

As the hole injection layer material, a known hole injection layer material may be used, for example, phthalocyanine compounds such as copper phthalocyanine, and the like, disclosed in U.S. Pat. No. 4,356,429, or starburst-type amine derivatives such as tris(4-carbazolyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), soluble conductive polymer polyaniline/dodecylbenzenesulfonic acid or poly(3,4-ethylenedioxythiophene)/poly(4-styrenesulfonate), polyaniline/camphor sulfonic acid or polyaniline/poly(4-styrene-sulfonate), and the like, disclosed in Advanced Material, 6, p. 677 (1994) may be used.

As the hole transport layer material, a pyrazoline derivative, an arylamine-based derivative, a stilbene derivative, a triphenyldiamine derivative, and the like may be used, and a low molecular weight or high molecular weight material may be used.

As the electron transport layer material, oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone and derivatives thereof, diphenyldicyanoethylene and derivatives thereof, diphenoquinone derivatives, metal complexes of 8-hydroxyquinoline and derivatives thereof, and the like may be used, and high molecular weight materials as well as low molecular weight materials may be used.

As the electron injection layer material, for example, LiF is typically used in the art, but the present invention is not limited thereto.

As the light-emitting layer material, a red, green, or blue light-emitting material may be used, and a mixture of two or more light-emitting materials may be used, if necessary. In this case, it is possible to use by depositing two or more light-emitting materials as separate sources, or it is possible to use by premixing and depositing them as a single source. In addition, as the light-emitting layer material, a fluorescent material may be used, or a phosphorescent material may be used. As the light-emitting layer material, materials that emit light by combining holes and electrons respectively injected from the anode and the cathode may be used alone, or materials in which the host material and the dopant material together participate in light emission may be used.

When using by mixing hosts of the light-emitting layer material, it is possible to use by mixing hosts of the same series, or it is possible to use by mixing hosts of different series. For example, it is possible to use by selecting any two or more types of n-type host material and p-type host material as the host material of the light-emitting layer.

The electronic-blocking layer material may include, but is limited to, one or more compounds selected from tris(phenyloyrazole) iridium, 9,9-bis[4-(N,N-bis-biphenyl-4-ylamino)phenyl]-9H-fluorene (BPAPF), bis[4-(p,p-ditolylamino)phenyl]diphenylsilane, NPD (4,4′-bis[N-(1-napthyl)-N-phenylamino]biphenyl), mCP (N,N′-dicarbazolyl-3,5-benzene), and MPMP (bis[4-(N,N-diethylamino)-2-methylphenyl](4-methylphenyl)methane).

In addition, the electron-blocking layer may include an inorganic compound. For example, it may include, but is not limited to, at least any one or a combination of halide compounds such as LiF, NaF, KF, RbF, CsF, FrF, MgF₂, CaF₂, SrF₂, BaF₂, LiCl, NaCl, KCl, RbCl, CsCl, FrCl, and the like; and oxides such as Li₂O, Li₂O₂, Na₂O, K₂O, Rb₂O, Rb₂O₂, Cs₂O, Cs₂O₂, LiAlO₂, LiBO₂, LiTaO₃, LiNbO₃, LiWO₄, Li₂CO, NaWO₄, KAlO₂, K₂SiO₃, B₂O₅, Al₂O₃, SiO₂, and the like.

The hole-blocking layer material may include, but is not limited to, an oxadiazole derivative, a triazole derivative, a phenanthroline derivative, BCP, an aluminum complex, and the like.

In the organic light-emitting device of the present invention, materials known in the art may be used without limitation as materials not described above.

The organic light-emitting device according to one embodiment of the present invention may be a top emission type, a bottom emission type, or a dual emission type depending on the material to be used.

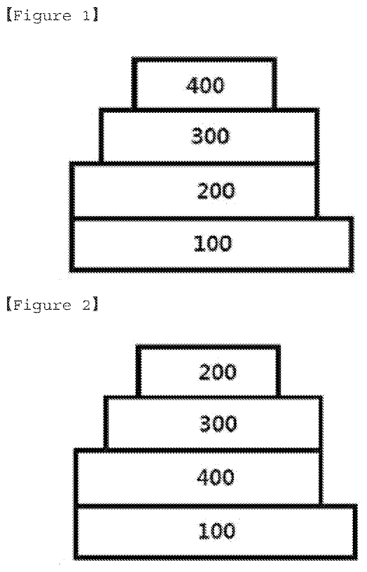

The accompanying FIGS. 1 to 3 illustrate the stack order of the electrodes and the organic layers of the organic light-emitting device according to one embodiment of the present invention. However, it is not intended that the scope of the present invention be limited by these drawings, and the structure of an organic light-emitting device known in the art may also be applied to the present invention.

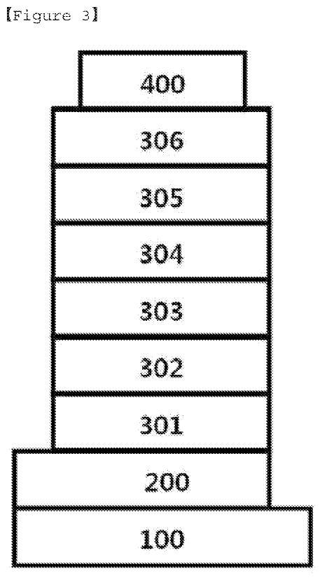

Referring to FIG. 1, an organic light-emitting device in which an anode 200, an organic layer 300, and a cathode 400 are sequentially stacked on a substrate 100 is illustrated. However, it is not limited to such a structure, and an organic light-emitting device in which a cathode, an organic layer, and an anode are sequentially stacked on a substrate may be implemented, as shown in FIG. 2.

FIG. 3 illustrates a case where the organic layer is multi-layered. The organic light-emitting device according to FIG. 3 comprises a hole injection layer 301, a hole transport layer 302, a light-emitting layer 303, a hole-blocking layer 304, an electron transport layer 305, and an electron injection layer 306. However, the scope of the present invention is not limited by such stack structures, and the remaining layers except for the light-emitting layer may be omitted, if necessary, and other necessary functional layers such as an electron-blocking layer may be further added.

In addition, the present invention relates to a composition for an organic layer of a organic light-emitting device comprising
the heterocyclic compound represented by Formula 1.

Specific contents of the heterocyclic compound represented by Formula 1 are the same as described above.

The composition for an organic layer may be used as a hole injection layer material, a hole transport layer material, an electron-blocking layer material, a light-emitting layer material, a hole-blocking layer material, an electron transport layer material, and an electron injection layer material, and in particular, may be preferably used as a hole transport layer material, an electron-blocking layer material, and a light-emitting layer material.

When it is used as a light-emitting layer material, it may be used as a host material, and it may be more usefully used as a p-Host (p-type host) having good hole transport ability among host materials.

The host material may include the composition for the organic layer alone, or may include the composition for the organic layer in combination with other host materials.

The composition for an organic layer may further include materials commonly used in the composition for an organic layer in the art, together with the heterocyclic compound represented by Formula 1.

In addition, the present invention relates to a method of manufacturing a organic light-emitting device, comprising the steps of:
preparing a substrate; forming a first electrode on the substrate; forming one or more organic layers on the first electrode; and forming a second electrode on the organic layer, wherein the step of forming the organic layers comprises the step of forming one or more organic layers using the hetero compound represented by Formula 1 or the composition for an organic layer of the present invention.

In one embodiment of the present invention, the step of forming the organic layers may form the organic layers using the hetero compound represented by Formula 1 or the composition for an organic material layer through a thermal vacuum deposition method.

The organic layer including the composition for an organic layer may further include other materials commonly used in the art, if necessary.

The heterocyclic compound represented by Formula 1 according to one embodiment of the present invention may act on a principle similar to that applied to the organic light-emitting device even in an organic electronic device including an organic solar cell, an organic photoreceptor, an organic transistor, and the like.

Hereinafter, preferred examples will be provided to help to understand the present invention, but the following examples are provided not to limit the present invention but to facilitate the understanding of the present invention.

### PREPARATIVE EXAMPLES

### [Preparative Example 1] Preparation of Compound 002

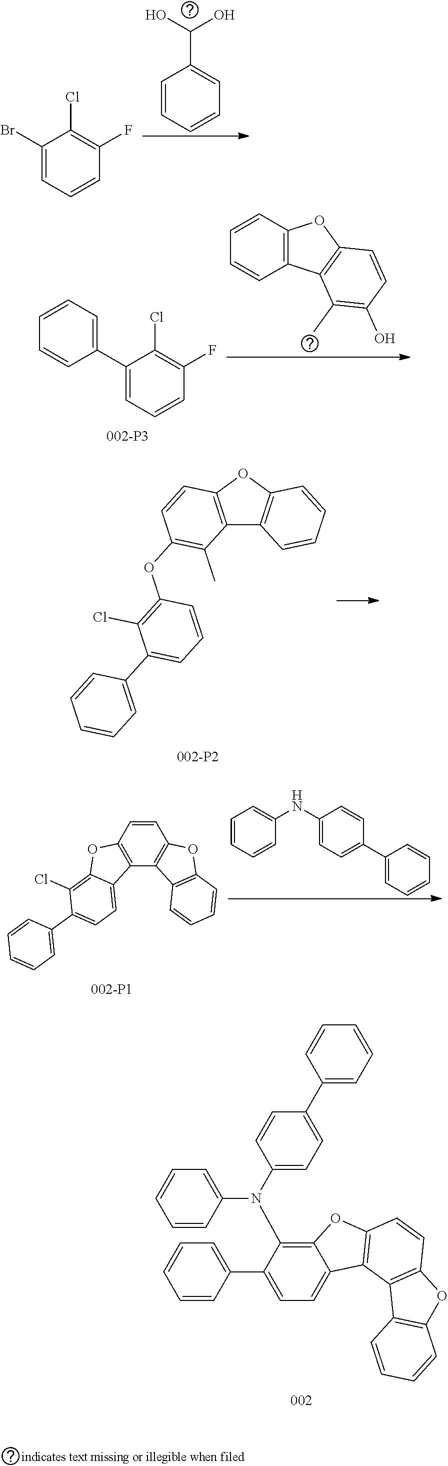

1) Preparation of Compound 002-P3

Compound 1-bromo-2-chloro-3-fluorobenzene (100 g, 477.46 mmol) and phenylboronic acid (61.13 g, 501.34 mmol) were dissolved in 1000 ml of toluene, 200 ml of ethanol, and 200 ml of distilled water, and then Pd(PPh₃)₄(27.59 g, 23.87 mmol) and K₂CO₃ (164.98 g, 1193.66 mmol) were placed therein and stirred under reflux for 12 hours. After completion of the reaction, ethyl acetate was placed and dissolved in the reaction solution, which was extracted with distilled water, and the organic layer was dried over anhydrous MgSO₄, and then the solvent was removed by a rotary evaporator and purified by column chromatography using dichloromethane and hexane as a developing solvent to give Compound 002-P3 (88 g, 89%).

2) Preparation of Compound 002-P2

Compound 002-P3 (88 g, 425.86 mmol) and 1-iododibenzo[b,d]furan-2-ol (145.26 g, 468.45 mmol) were dissolved in 1000 ml of N,N-dimethylacetamide and heated to 150° C., and then Cs₂CO (277.51 g, 851.72 mmol) was placed therein and stirred under reflux for 30 minutes. After completion of the reaction, it was extracted with dichloromethane and distilled water, and the organic layer was dried over anhydrous MgSO₄, and then the solvent was removed by a rotary evaporator and purified by column chromatography using dichloromethane and hexane as a developing solvent to give Compound 002-P2 (130 g, 61%).

3) Preparation of Compound 002-P1

Compound 002-P2 (130 g, 261.72 mmol) was dissolved in 1-methyl-2-pyrrolidinone, and then Pd(PPh₃)₄(15.12 g, 13.09 mmol), PPh₃ (6.86 g, 26.17 mmol), and Na₂CO₃ (55.48 g, 523.43 mmol) were placed therein and stirred under reflux for 12 hours. After completion of the reaction, it was extracted with dichloromethane and distilled water, and the organic layer was dried over anhydrous MgSO₄, and then the solvent was removed by a rotary evaporator and purified by column chromatography using dichloromethane and hexane as a developing solvent to give Compound 002-P1 (65 g, 67%).

4) Preparation of Compound 002

Compound 002-P1 (10 g, 27.11 mmol) and N-phenyl-[1,1′-biphenyl]-4-amine (6.98 g, 28.47 mmol) were dissolved in 100 ml of Xylene, and then Pd₂(dba)₃ (1.24 g, 1.36 mmol), P(t-Bu)₃ (1.26 ml, 2.71 mmol), and t-BuONa (6.51 g, 67.79 mmol) were placed therein and stirred under reflux for 3 hours. After completion of the reaction, MC was placed and dissolved in the reaction solution, which was extracted with distilled water, and the organic layer was dried over anhydrous MgSO₄, and then the solvent was removed by a rotary evaporator and purified by column chromatography using dichloromethane and hexane as a developing solvent to give Compound 002 (12 g, 77%).

The following Table 1 shows that the target compound was synthesized in the same manner as in Preparative Example 1, except that Compound A instead of 1-bromo-2-chloro-3-fluorobenzene, Compound B instead of phenylboronic acid, and Compound C instead of N-phenyl-[1,1′-biphenyl]-4-amine in Preparative Example 1 were used.

TABLE 1

| Com- | | | | | |
|---|---|---|---|---|---|
| pound | | | | | |
| No. | Compound A | Compound B | Compound C | Target Compound | Yield |
| 005 | 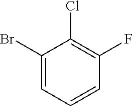 | 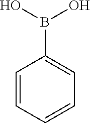 | 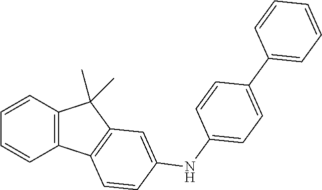 [SMILES: CC1(C)c2ccccc2-c2ccc(Nc3ccc(-c4ccccc4)cc3)cc21] | 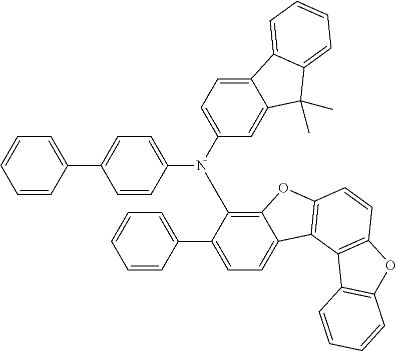 [SMILES: CC1(C)c2ccccc2-c2ccc(N(c3ccc(-c4ccccc4)cc3)c3c(-c4ccccc4)ccc4c3oc3ccc5oc6ccccc6c5c34)cc21] | 69% |
| 019 | 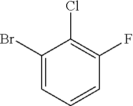 | 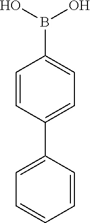 [SMILES: OB(O)c1ccc(-c2ccccc2)cc1] | 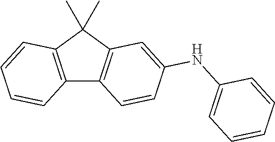 [SMILES: CC1(C)c2ccccc2-c2ccc(Nc3ccccc3)cc21] | 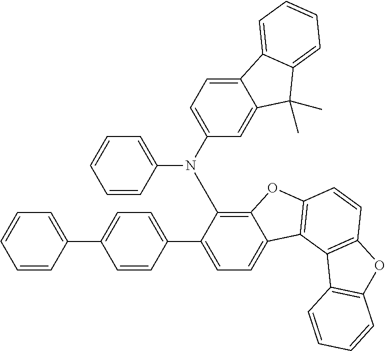 [SMILES: CC1(C)c2ccccc2-c2ccc(N(c3ccccc3)c3c(-c4ccc(-c5ccccc5)cc4)ccc4c3oc3ccc5oc6ccccc6c5c34)cc21] | 73% |
| 033 | 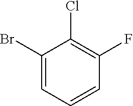 | 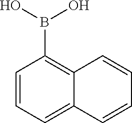 [SMILES: OB(O)c1cccc2ccccc12] | 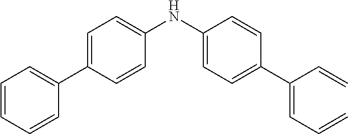 [SMILES: C=C/C=C(\C=C)c1ccc(Nc2ccc(-c3ccccc3)cc2)cc1] | 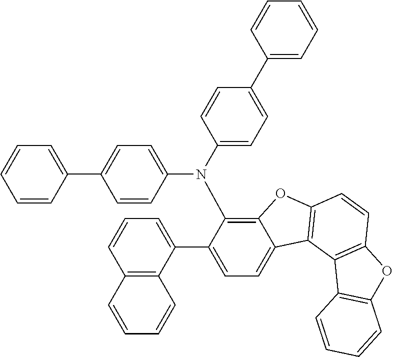 [SMILES: c1ccc(-c2ccc(N(c3ccc(-c4ccccc4)cc3)c3c(-c4cccc5ccccc45)ccc4c3oc3ccc5oc6ccccc6c5c34)cc2)cc1] | 72% |
| 052 | 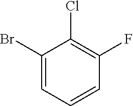 | 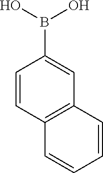 [SMILES: OB(O)c1ccc2ccccc2c1] | 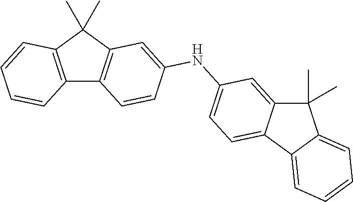 [SMILES: CC1(C)c2ccccc2-c2ccc(Nc3ccc4c(c3)C(C)(C)c3ccccc3-4)cc21] | 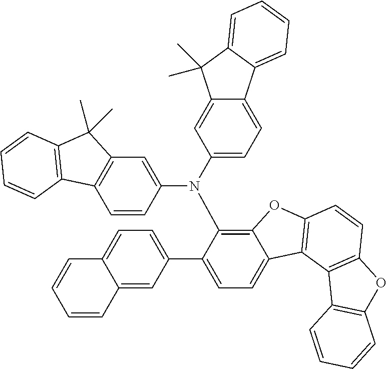 [SMILES: CC1(C)c2ccccc2-c2ccc(N(c3ccc4c(c3)C(C)(C)c3ccccc3-4)c3c(-c4ccc5ccccc5c4)ccc4c3oc3ccc5oc6ccccc6c5c34)cc21] | 65% |
| 080 | 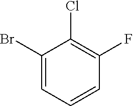 | 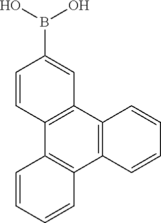 [SMILES: OB(O)c1ccc2c3ccccc3c3ccccc3c2c1] | 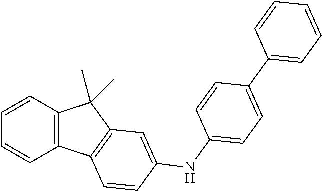 [SMILES: CC1(C)c2ccccc2-c2ccc(Nc3ccc(-c4ccccc4)cc3)cc21] | 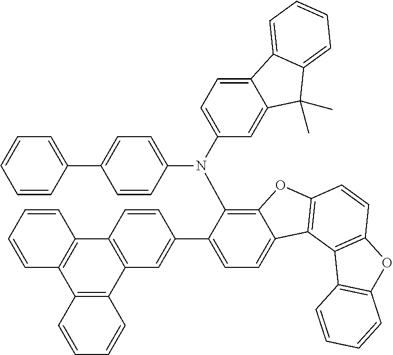 [SMILES: CC1(C)c2ccccc2-c2ccc(N(c3ccc(-c4ccccc4)cc3)c3c(-c4ccc5c6ccccc6c6ccccc6c5c4)ccc4c3oc3ccc5oc6ccccc6c5c34)cc21] | 70% |
| 098 | 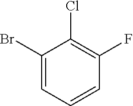 | 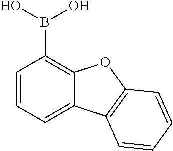 [SMILES: OB(O)c1cccc2c1oc1ccccc12] | 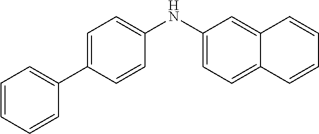 [SMILES: c1ccc(-c2ccc(Nc3ccc4ccccc4c3)cc2)cc1] | 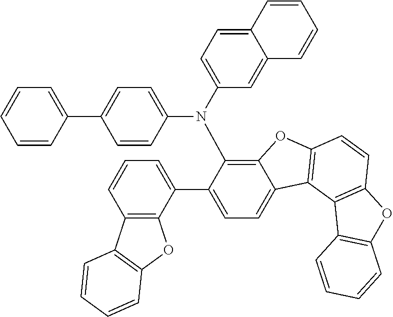 [SMILES: c1ccc(-c2ccc(N(c3ccc4ccccc4c3)c3c(-c4cccc5c4oc4ccccc45)ccc4c3oc3ccc5oc6ccccc6c5c34)cc2)cc1] | 69% |
| 106 | 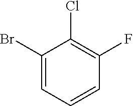 | 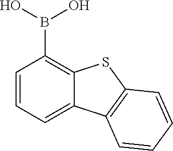 [SMILES: OB(O)c1cccc2c1sc1ccccc12] | 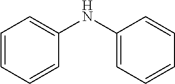 [SMILES: c1ccc(Nc2ccccc2)cc1] | 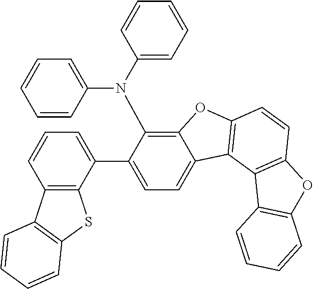 [SMILES: c1ccc(N(c2ccccc2)c2c(-c3cccc4c3sc3ccccc34)ccc3c2oc2ccc4oc5ccccc5c4c23)cc1] | 76% |
| 123 | 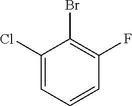 | 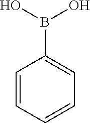 | 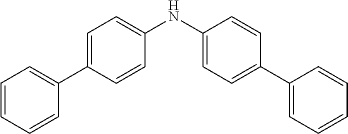 [SMILES: c1ccc(-c2ccc(Nc3ccc(-c4ccccc4)cc3)cc2)cc1] | 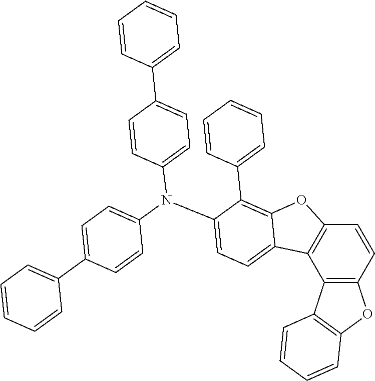 [SMILES: c1ccc(-c2ccc(N(c3ccc(-c4ccccc4)cc3)c3ccc4c(oc5ccc6oc7ccccc7c6c54)c3-c3ccccc3)cc2)cc1] | 72% |
| 125 | 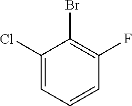 | 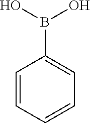 | 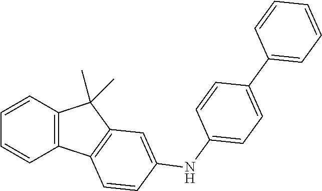 [SMILES: CC1(C)c2ccccc2-c2ccc(Nc3ccc(-c4ccccc4)cc3)cc21] | 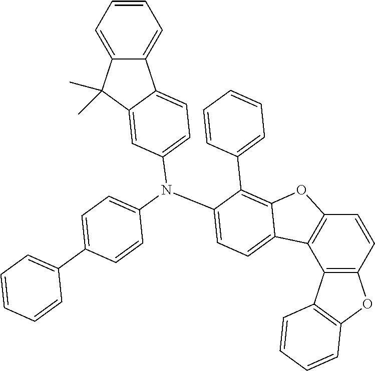 [SMILES: CC1(C)c2ccccc2-c2ccc(N(c3ccc(-c4ccccc4)cc3)c3ccc4c(oc5ccc6oc7ccccc7c6c54)c3-c3ccccc3)cc21] | 73% |
| 136 | 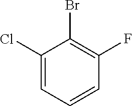 | 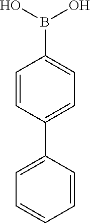 [SMILES: OB(O)c1ccc(-c2ccccc2)cc1] | 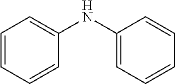 [SMILES: c1ccc(Nc2ccccc2)cc1] | 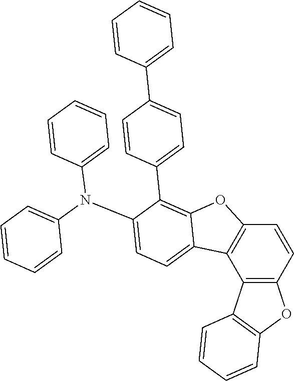 [SMILES: c1ccc(-c2ccc(-c3c(N(c4ccccc4)c4ccccc4)ccc4c3oc3ccc5oc6ccccc6c5c34)cc2)cc1] | 78% |
| 160 | 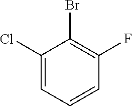 | 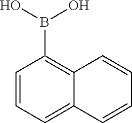 [SMILES: OB(O)c1cccc2ccccc12] | 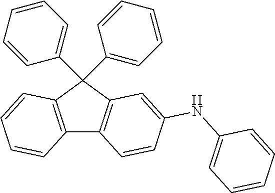 [SMILES: c1ccc(Nc2ccc3c(c2)C(c2ccccc2)(c2ccccc2)c2ccccc2-3)cc1] | 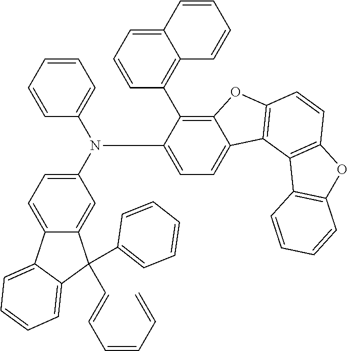 [SMILES: C=C/C=C\C=C\C1(c2ccccc2)c2ccccc2-c2ccc(N(c3ccccc3)c3ccc4c(oc5ccc6oc7ccccc7c6c54)c3-c3cccc4ccccc34)cc21] | 77% |
| 168 | 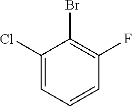 | 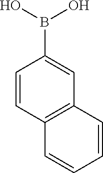 [SMILES: OB(O)c1ccc2ccccc2c1] | 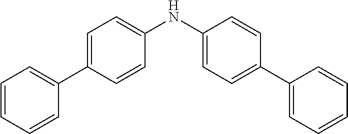 [SMILES: c1ccc(-c2ccc(Nc3ccc(-c4ccccc4)cc3)cc2)cc1] | 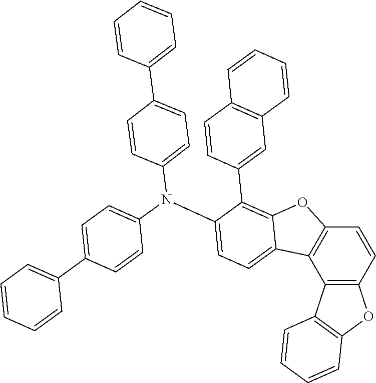 [SMILES: c1ccc(-c2ccc(N(c3ccc(-c4ccccc4)cc3)c3ccc4c(oc5ccc6oc7ccccc7c6c54)c3-c3ccc4ccccc4c3)cc2)cc1] | 77% |
| 211 | 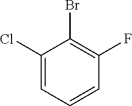 | 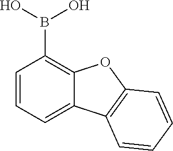 [SMILES: OB(O)c1cccc2c1oc1ccccc12] | 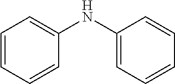 [SMILES: c1ccc(Nc2ccccc2)cc1] | 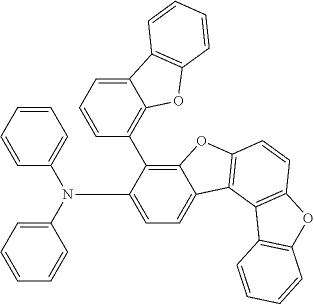 [SMILES: c1ccc(N(c2ccccc2)c2ccc3c(oc4ccc5oc6ccccc6c5c43)c2-c2cccc3c2oc2ccccc23)cc1] | 81% |
| 229 | 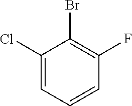 | 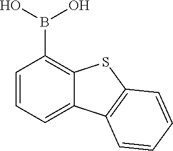 [SMILES: OB(O)c1cccc2c1sc1ccccc12] | 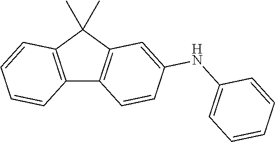 [SMILES: CC1(C)c2ccccc2-c2ccc(Nc3ccccc3)cc21] | 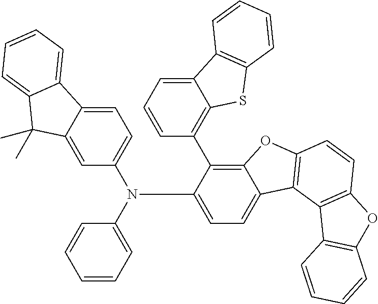 [SMILES: CC1(C)c2ccccc2-c2ccc(N(c3ccccc3)c3ccc4c(oc5ccc6oc7ccccc7c6c54)c3-c3cccc4c3sc3ccccc34)cc21] | 71% |
| 243 | 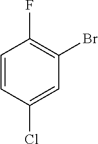 | 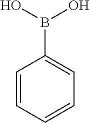 | 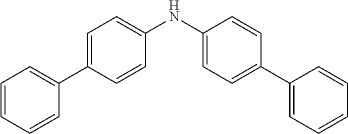 [SMILES: c1ccc(-c2ccc(Nc3ccc(-c4ccccc4)cc3)cc2)cc1] | 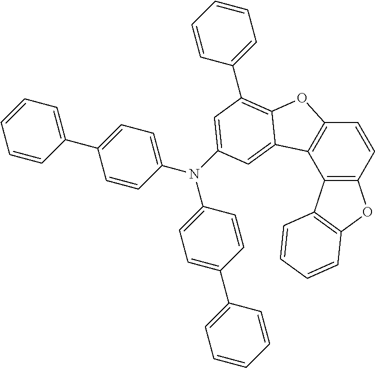 [SMILES: c1ccc(-c2ccc(N(c3ccc(-c4ccccc4)cc3)c3cc(-c4ccccc4)c4oc5ccc6oc7ccccc7c6c5c4c3)cc2)cc1] | 75% |
| 247 | 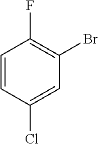 | 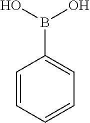 | 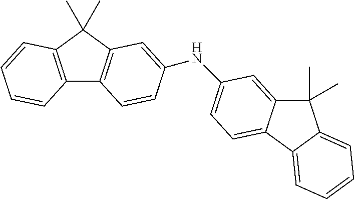 [SMILES: CC1(C)c2ccccc2-c2ccc(Nc3ccc4c(c3)C(C)(C)c3ccccc3-4)cc21] | 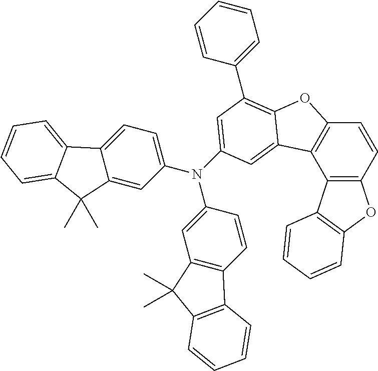 [SMILES: CC1(C)c2ccccc2-c2ccc(N(c3ccc4c(c3)C(C)(C)c3ccccc3-4)c3cc(-c4ccccc4)c4oc5ccc6oc7ccccc7c6c5c4c3)cc21] | 82% |
| 257 | 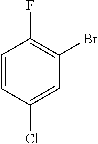 | 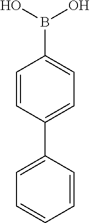 [SMILES: OB(O)c1ccc(-c2ccccc2)cc1] | 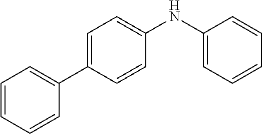 [SMILES: c1ccc(Nc2ccc(-c3ccccc3)cc2)cc1] | 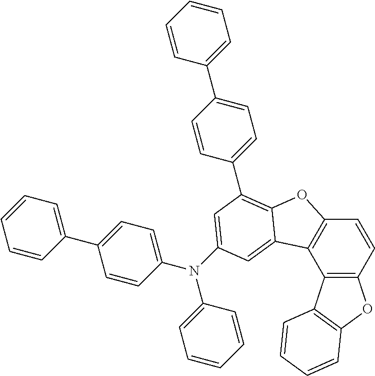 [SMILES: c1ccc(-c2ccc(-c3cc(N(c4ccccc4)c4ccc(-c5ccccc5)cc4)cc4c3oc3ccc5oc6ccccc6c5c34)cc2)cc1] | 71% |
| 279 | 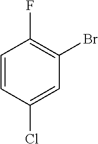 | 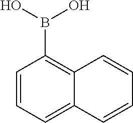 [SMILES: OB(O)c1cccc2ccccc12] | 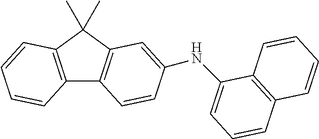 [SMILES: CC1(C)c2ccccc2-c2ccc(Nc3cccc4ccccc34)cc21] | 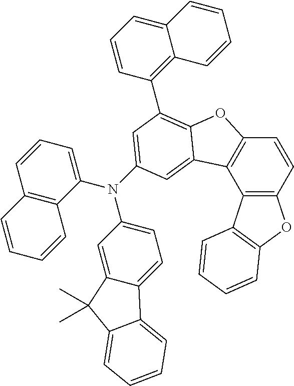 [SMILES: CC1(C)c2ccccc2-c2ccc(N(c3cc(-c4cccc5ccccc45)c4oc5ccc6oc7ccccc7c6c5c4c3)c3cccc4ccccc34)cc21] | 78% |
| 292 | 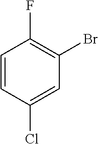 | 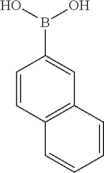 [SMILES: OB(O)c1ccc2ccccc2c1] | 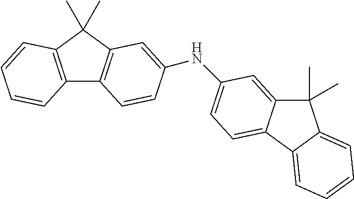 [SMILES: CC1(C)c2ccccc2-c2ccc(Nc3ccc4c(c3)C(C)(C)c3ccccc3-4)cc21] | 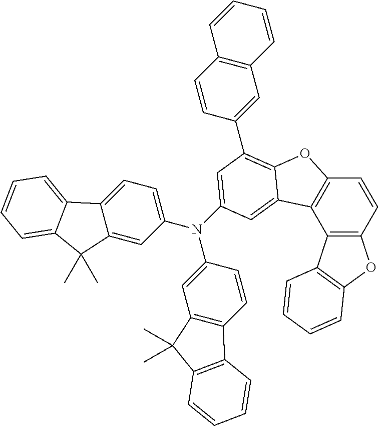 [SMILES: CC1(C)c2ccccc2-c2ccc(N(c3ccc4c(c3)C(C)(C)c3ccccc3-4)c3cc(-c4ccc5ccccc5c4)c4oc5ccc6oc7ccccc7c6c5c4c3)cc21] | 72% |
| 311 | 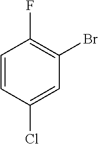 | 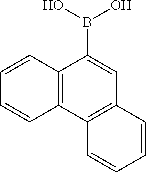 [SMILES: OB(O)c1cc2ccccc2c2ccccc12] | 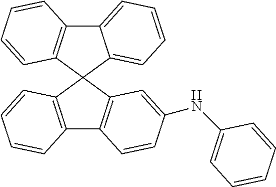 [SMILES: c1ccc(Nc2ccc3c(c2)C2(c4ccccc4-c4ccccc42)c2ccccc2-3)cc1] | 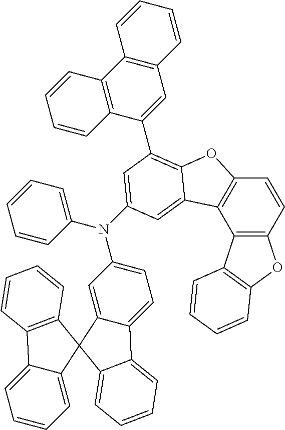 [SMILES: c1ccc(N(c2ccc3c(c2)C2(c4ccccc4-c4ccccc42)c2ccccc2-3)c2cc(-c3cc4ccccc4c4ccccc34)c3oc4ccc5oc6ccccc6c5c4c3c2)cc1] | 76% |
| 334 | 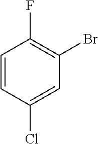 | 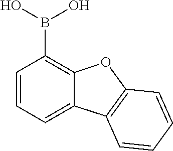 [SMILES: OB(O)c1cccc2c1oc1ccccc12] | 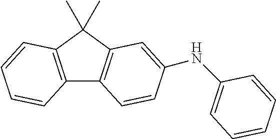 [SMILES: CC1(C)c2ccccc2-c2ccc(Nc3ccccc3)cc21] | 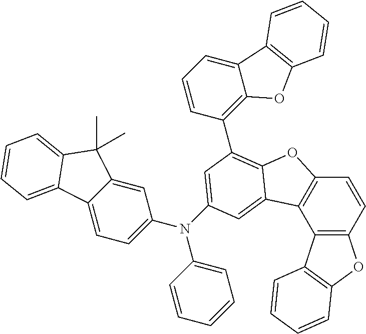 [SMILES: CC1(C)c2ccccc2-c2ccc(N(c3ccccc3)c3cc(-c4cccc5c4oc4ccccc45)c4oc5ccc6oc7ccccc7c6c5c4c3)cc21] | 68% |
| 347 | 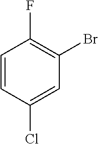 | 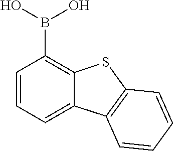 [SMILES: OB(O)c1cccc2c1sc1ccccc12] | 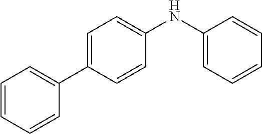 [SMILES: c1ccc(Nc2ccc(-c3ccccc3)cc2)cc1] | 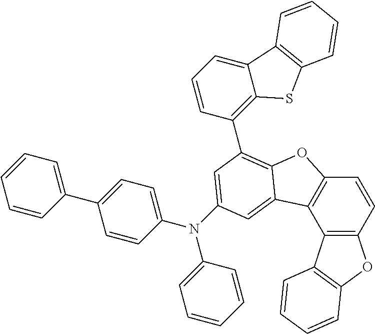 [SMILES: c1ccc(-c2ccc(N(c3ccccc3)c3cc(-c4cccc5c4sc4ccccc45)c4oc5ccc6oc7ccccc7c6c5c4c3)cc2)cc1] | 73% |
| 363 | 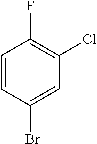 | 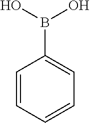 | 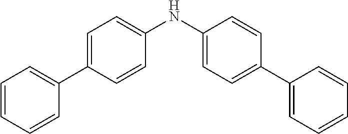 [SMILES: c1ccc(-c2ccc(Nc3ccc(-c4ccccc4)cc3)cc2)cc1] | 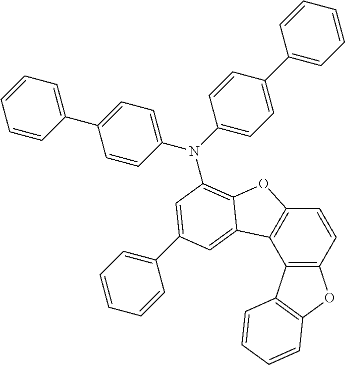 [SMILES: c1ccc(-c2ccc(N(c3ccc(-c4ccccc4)cc3)c3cc(-c4ccccc4)cc4c3oc3ccc5oc6ccccc6c5c34)cc2)cc1] | 74% |
| 365 | 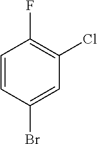 | 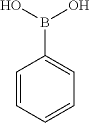 | 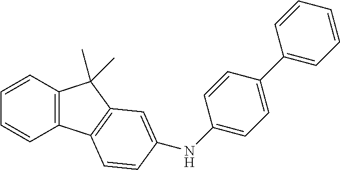 [SMILES: CC1(C)c2ccccc2-c2ccc(Nc3ccc(-c4ccccc4)cc3)cc21] | 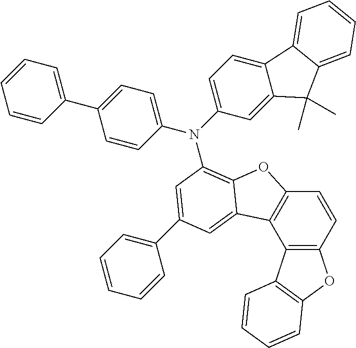 [SMILES: CC1(C)c2ccccc2-c2ccc(N(c3ccc(-c4ccccc4)cc3)c3cc(-c4ccccc4)cc4c3oc3ccc5oc6ccccc6c5c34)cc21] | 79% |
| 371 | 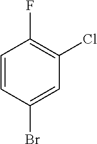 | 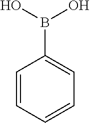 | 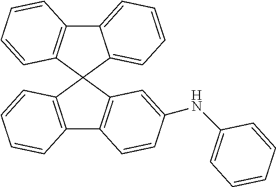 [SMILES: c1ccc(Nc2ccc3c(c2)C2(c4ccccc4-c4ccccc42)c2ccccc2-3)cc1] | 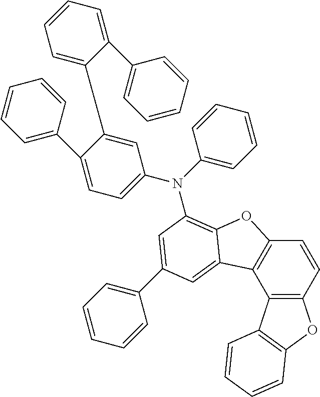 [SMILES: c1ccc(-c2cc(N(c3ccccc3)c3ccc(-c4ccccc4)c(-c4ccccc4-c4ccccc4)c3)c3oc4ccc5oc6ccccc6c5c4c3c2)cc1] | 71% |
| 378 | 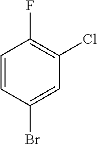 | 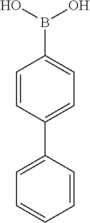 [SMILES: OB(O)c1ccc(-c2ccccc2)cc1] | 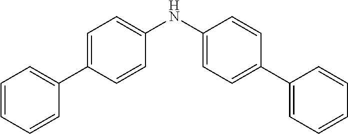 [SMILES: c1ccc(-c2ccc(Nc3ccc(-c4ccccc4)cc3)cc2)cc1] | 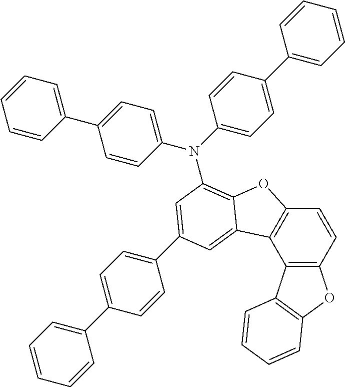 [SMILES: c1ccc(-c2ccc(-c3cc(N(c4ccc(-c5ccccc5)cc4)c4ccc(-c5ccccc5)cc4)c4oc5ccc6oc7ccccc7c6c5c4c3)cc2)cc1] | 73% |
| 395 | 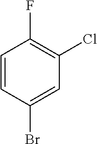 | 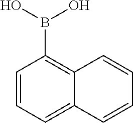 [SMILES: OB(O)c1cccc2ccccc12] | 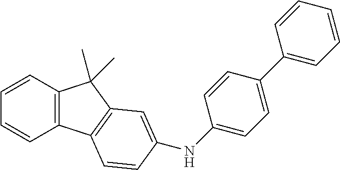 [SMILES: CC1(C)c2ccccc2-c2ccc(Nc3ccc(-c4ccccc4)cc3)cc21] | 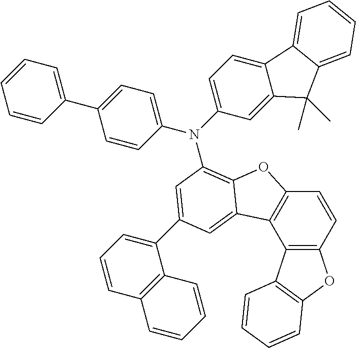 [SMILES: CC1(C)c2ccccc2-c2ccc(N(c3ccc(-c4ccccc4)cc3)c3cc(-c4cccc5ccccc45)cc4c3oc3ccc5oc6ccccc6c5c34)cc21] | 77% |
| 411 | 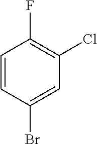 | 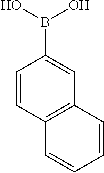 [SMILES: OB(O)c1ccc2ccccc2c1] | 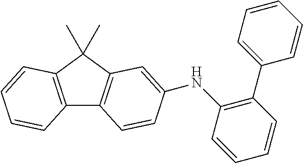 [SMILES: CC1(C)c2ccccc2-c2ccc(Nc3ccccc3-c3ccccc3)cc21] | 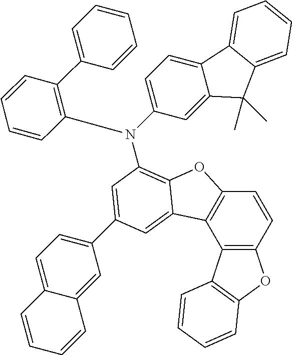 [SMILES: CC1(C)c2ccccc2-c2ccc(N(c3ccccc3-c3ccccc3)c3cc(-c4ccc5ccccc5c4)cc4c3oc3ccc5oc6ccccc6c5c34)cc21] | 74% |
| 457 | 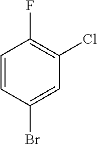 | 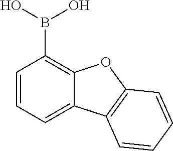 [SMILES: OB(O)c1cccc2c1oc1ccccc12] | 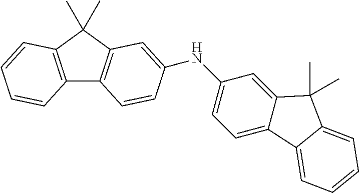 [SMILES: CC1(C)c2ccccc2-c2ccc(Nc3ccc4c(c3)C(C)(C)c3ccccc3-4)cc21] | 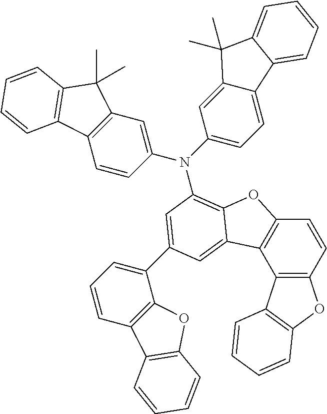 [SMILES: CC1(C)c2ccccc2-c2ccc(N(c3ccc4c(c3)C(C)(C)c3ccccc3-4)c3cc(-c4cccc5c4oc4ccccc45)cc4c3oc3ccc5oc6ccccc6c5c34)cc21] | 76% |
| 603 | 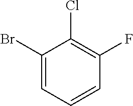 | 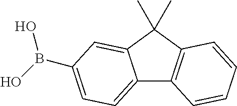 [SMILES: CC1(C)c2ccccc2-c2ccc(B(O)O)cc21] | 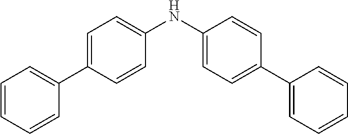 [SMILES: c1ccc(-c2ccc(Nc3ccc(-c4ccccc4)cc3)cc2)cc1] | 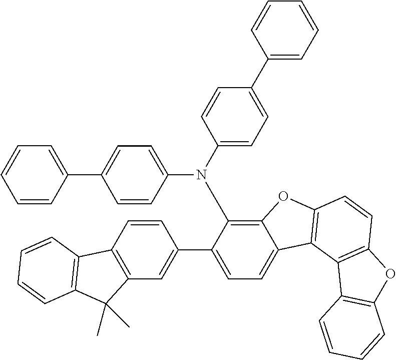 [SMILES: CC1(C)c2ccccc2-c2ccc(-c3ccc4c(oc5ccc6oc7ccccc7c6c54)c3N(c3ccc(-c4ccccc4)cc3)c3ccc(-c4ccccc4)cc3)cc21] | 75% |
| 605 | 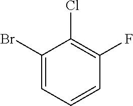 | 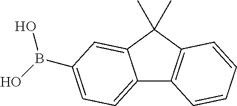 [SMILES: CC1(C)c2ccccc2-c2ccc(B(O)O)cc21] | 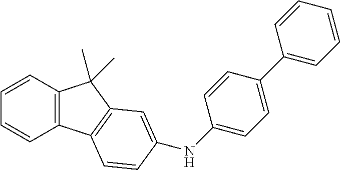 [SMILES: CC1(C)c2ccccc2-c2ccc(Nc3ccc(-c4ccccc4)cc3)cc21] | 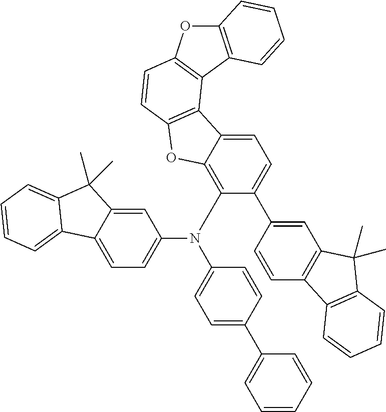 [SMILES: CC1(C)c2ccccc2-c2ccc(-c3ccc4c(oc5ccc6oc7ccccc7c6c54)c3N(c3ccc(-c4ccccc4)cc3)c3ccc4c(c3)C(C)(C)c3ccccc3-4)cc21] | 71% |
| 632 | 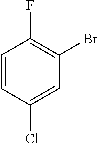 | 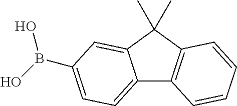 [SMILES: CC1(C)c2ccccc2-c2ccc(B(O)O)cc21] | 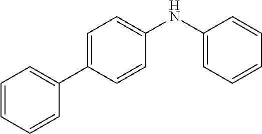 [SMILES: c1ccc(Nc2ccc(-c3ccccc3)cc2)cc1] | 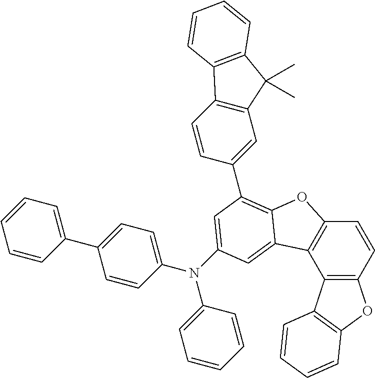 [SMILES: CC1(C)c2ccccc2-c2ccc(-c3cc(N(c4ccccc4)c4ccc(-c5ccccc5)cc4)cc4c3oc3ccc5oc6ccccc6c5c34)cc21] | 71% |
| 651 | 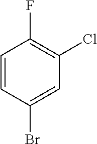 | 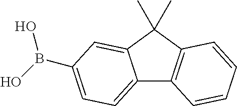 [SMILES: CC1(C)c2ccccc2-c2ccc(B(O)O)cc21] | 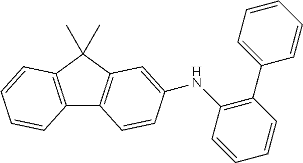 [SMILES: CC1(C)c2ccccc2-c2ccc(Nc3ccccc3-c3ccccc3)cc21] | 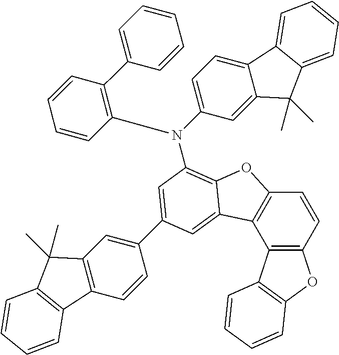 [SMILES: CC1(C)c2ccccc2-c2ccc(-c3cc(N(c4ccc5c(c4)C(C)(C)c4ccccc4-5)c4ccccc4-c4ccccc4)c4oc5ccc6oc7ccccc7c6c5c4c3)cc21] | 75% |

### [Preparative Example 2] Preparation of Compound 012

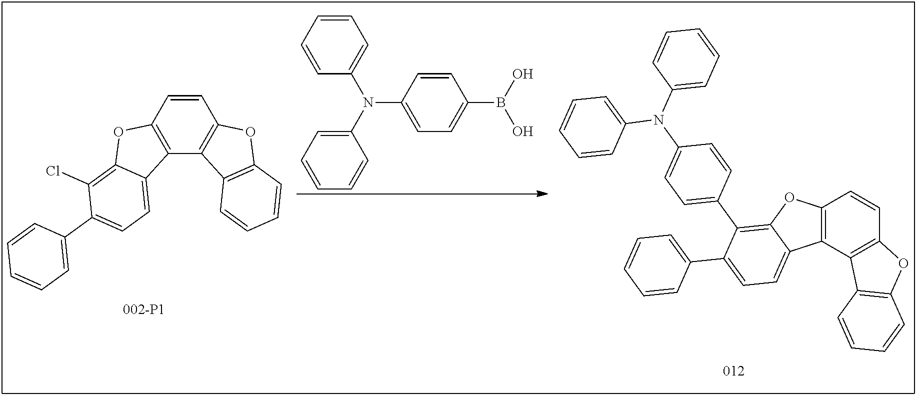

1) Preparation of Compound 012 Compound 002-P1 (10 g, 27.11 mmol) and (4-(diphenylamino)phenyl)boronic acid (8.23 g, 28.47 mmol) were dissolved in 100 ml of 1,4-dioxane and 20 ml of H₂O, and then Pd(dba)₂ (0.78 g, 1.36 mmol), xphos (1.29 g, 2.71 mmol), and K₂CO₃ (9.37 g, 67.79 mmol) were placed therein and stirred under reflux for 3 hours. After completion of the reaction, MC was placed and dissolved in the reaction solution, which was extracted with distilled water, and the organic layer was dried over anhydrous MgSO₄, and then the solvent was removed by a rotary evaporator and purified by column chromatography using dichloromethane and hexane as a developing solvent to give Compound 012 (11 g, 70%).

The following Table 2 shows that the target compound was synthesized in the same manner as in Preparative Example 1, except that Compound D instead of 002-P1 and Compound E instead of (4-(diphenylamino)phenyl)boronic acid in Preparative Example 1 were used.

TABLE 2

| Com- | | | | |
|---|---|---|---|---|
| pound | | | | |
| No. | Compound D | Compound E | Target Compound | Yield |
| 015 | 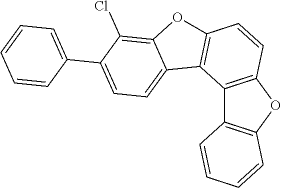 [SMILES: Clc1c(-c2ccccc2)ccc2c1oc1ccc3oc4ccccc4c3c12] | 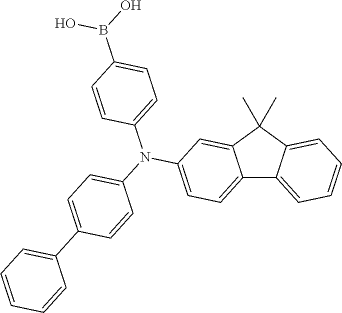 [SMILES: CC1(C)c2ccccc2-c2ccc(N(c3ccc(B(O)O)cc3)c3ccc(-c4ccccc4)cc3)cc21] | 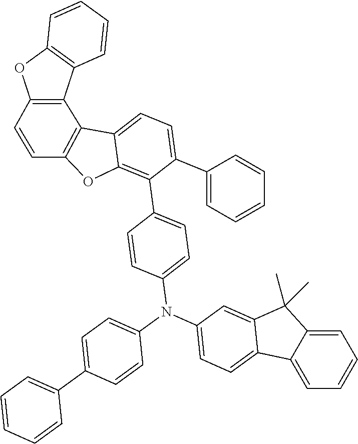 [SMILES: CC1(C)c2ccccc2-c2ccc(N(c3ccc(-c4ccccc4)cc3)c3ccc(-c4c(-c5ccccc5)ccc5c4oc4ccc6oc7ccccc7c6c45)cc3)cc21] | 71% |
| 134 | 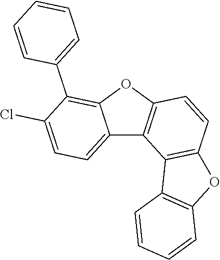 [SMILES: Clc1ccc2c(oc3ccc4oc5ccccc5c4c32)c1-c1ccccc1] | 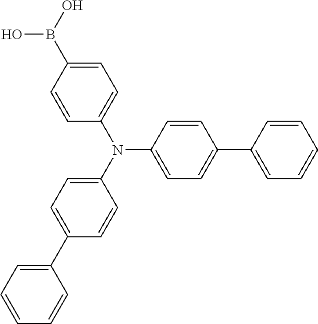 [SMILES: OB(O)c1ccc(N(c2ccc(-c3ccccc3)cc2)c2ccc(-c3ccccc3)cc2)cc1] | 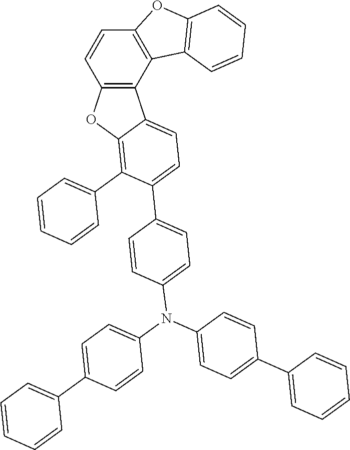 [SMILES: c1ccc(-c2ccc(N(c3ccc(-c4ccccc4)cc3)c3ccc(-c4ccc5c(oc6ccc7oc8ccccc8c7c65)c4-c4ccccc4)cc3)cc2)cc1] | 79% |
| 255 | 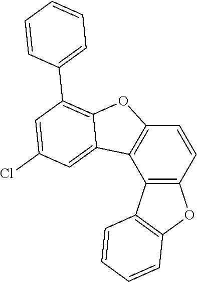 [SMILES: Clc1cc(-c2ccccc2)c2oc3ccc4oc5ccccc5c4c3c2c1] | 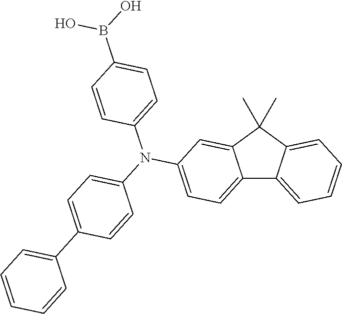 [SMILES: CC1(C)c2ccccc2-c2ccc(N(c3ccc(B(O)O)cc3)c3ccc(-c4ccccc4)cc3)cc21] | 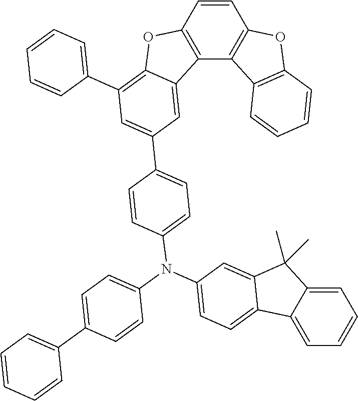 [SMILES: CC1(C)c2ccccc2-c2ccc(N(c3ccc(-c4ccccc4)cc3)c3ccc(-c4cc(-c5ccccc5)c5oc6ccc7oc8ccccc8c7c6c5c4)cc3)cc21] | 72% |
| 267 | 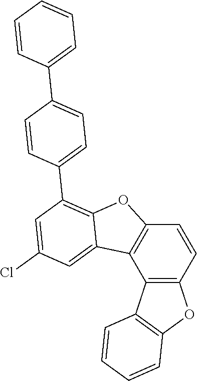 [SMILES: Clc1cc(-c2ccc(-c3ccccc3)cc2)c2oc3ccc4oc5ccccc5c4c3c2c1] | 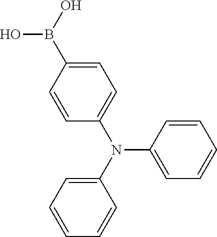 [SMILES: OB(O)c1ccc(N(c2ccccc2)c2ccccc2)cc1] | 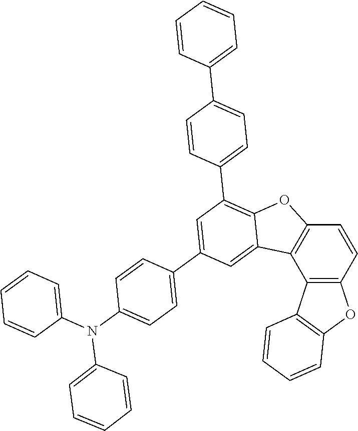 [SMILES: c1ccc(-c2ccc(-c3cc(-c4ccc(N(c5ccccc5)c5ccccc5)cc4)cc4c3oc3ccc5oc6ccccc6c5c34)cc2)cc1] | 71% |
| 299 | 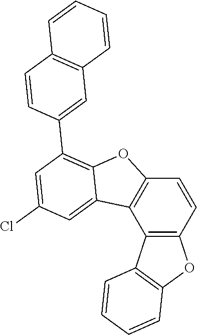 [SMILES: Clc1cc(-c2ccc3ccccc3c2)c2oc3ccc4oc5ccccc5c4c3c2c1] | 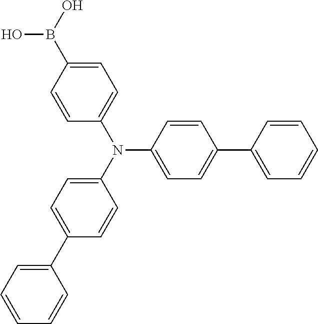 [SMILES: OB(O)c1ccc(N(c2ccc(-c3ccccc3)cc2)c2ccc(-c3ccccc3)cc2)cc1] | 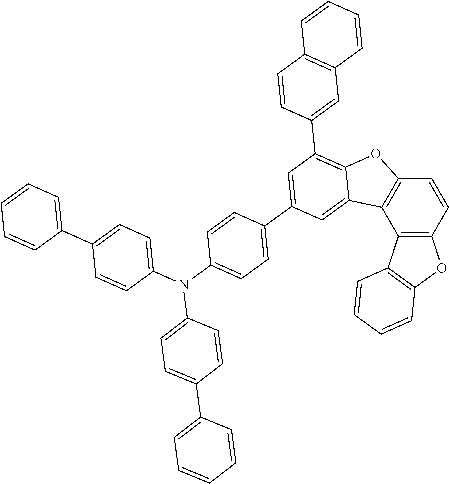 [SMILES: c1ccc(-c2ccc(N(c3ccc(-c4ccccc4)cc3)c3ccc(-c4cc(-c5ccc6ccccc6c5)c5oc6ccc7oc8ccccc8c7c6c5c4)cc3)cc2)cc1] | 79% |
| 375 | 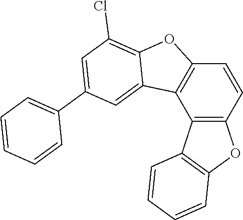 [SMILES: Clc1cc(-c2ccccc2)cc2c1oc1ccc3oc4ccccc4c3c12] | 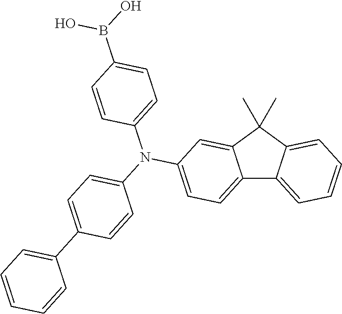 [SMILES: CC1(C)c2ccccc2-c2ccc(N(c3ccc(B(O)O)cc3)c3ccc(-c4ccccc4)cc3)cc21] | 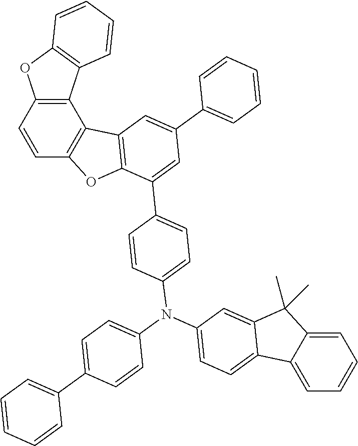 [SMILES: CC1(C)c2ccccc2-c2ccc(N(c3ccc(-c4ccccc4)cc3)c3ccc(-c4cc(-c5ccccc5)cc5c4oc4ccc6oc7ccccc7c6c45)cc3)cc21] | 70% |
| 657 | 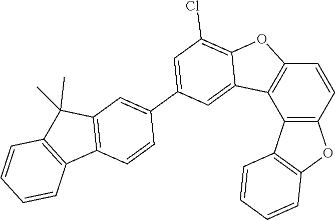 [SMILES: CC1(C)c2ccccc2-c2ccc(-c3cc(Cl)c4oc5ccc6oc7ccccc7c6c5c4c3)cc21] | 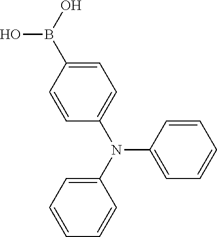 [SMILES: OB(O)c1ccc(N(c2ccccc2)c2ccccc2)cc1] | 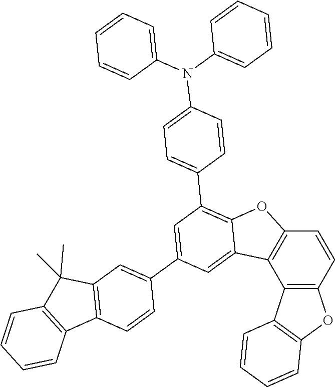 [SMILES: CC1(C)c2ccccc2-c2ccc(-c3cc(-c4ccc(N(c5ccccc5)c5ccccc5)cc4)c4oc5ccc6oc7ccccc7c6c5c4c3)cc21] | 75% |

Compounds were prepared in the same manner as in the above preparative examples and the synthesis confirmation results are shown in Tables 3 and 4. Table 3 shows the measured values of 1H NMR (CDCl₃, 300 MHz) and Table 4 shows the measured values of field desorption mass spectrometry (FD-MS).

TABLE 3

| Com- | |
|---|---|
| pound | ¹H NMR (CDCl₃, 300 MHz) |
| 002 | δ = 7.89 1H, d), 7.66(2H, d), 7.54-7.31(15H, m), 7.20(2H, |
| | t), 7.08(2H, d), 6.81(1H, t), 6.69-6.63(4H, m) |
| 005 | δ = 7.89-7.87(2H, m), 7.66-7.28(21H, m), 7.08(2H, d), |
| | 6.75(1H, s), 6.69(2H, d), 6.58(1H, d), 1.72(6H, s) |
| 012 | δ = 7.91-7.89(2H, m), 7.81-7.79(3H, m) 7.66(2H, d), 7.54- |
| | 7.32(8H, m), 7.20(4H, t), 6.81(2H, t), 6.69-6.63(6H, m) |
| 015 | δ = 7.91-7.79(6H, m), 7.66-7.28(21H, m), 6.75(1H, s), |
| | 6.69(4H, d), 6.58(1H, d), 1.72(6H, s) |
| 019 | δ = 7.89-7.87(2H, m), 7.66-7.62(3H, m), 7.55-7.20(19H, m), |
| | 6.81(1H, t), 6.75(1H, s), 6.63(2H, d), 6.58(1H, d), |
| | 1.72(6H, s) |
| 033 | δ = 8.55(1H, d), 8.42(1H, d), 8.08-8.04(2H, m), 7.89(1H, |
| | d), 7.66-7.31(24H, m), 6.69(4H, d) |
| 052 | δ = 8.00(2H, d), 7.92-7.87(4H, m), 7.73(1H, d), 7.66- |
| | 7.50(10H, m), 7.43-7.28(8H, m), 6.75(2H, s), 6.58(2H, d), |
| | 1.72(12H, s) |
| 080 | δ = 9.15(1H, s), 8.93(2H, d), 8.18(1H, d), 8.12(2H, d), |
| | 8.04(1H, d), 7.89-7.82(6H, m), 7.66-7.28(18H, m), 6.75(1H, |
| | s), 6.69(2H, d), 6.58(1H, d), 1.72(6H, s) |
| 098 | δ = 7.89-7.74(8H, m), 7.66(3H, s), 7.54-7.31(18H, m), |
| | 6.69(2H, d) |
| 106 | δ = 8.45-8.41(2H, m), 8.20(1H, d), 7.98(1H, d), 7.89(1H, |
| | d), 7.66-7.20(10H, m), 7.20(4H, t), 6.81(2H, t), 6.63(4H, |
| | d) |
| 123 | δ = 7.89(1H, d), 7.66-7.32(25H, m), 6.69(4H, d), 6.39(1H, |
| | d) |
| 125 | δ = 7.89-7.87(2H, m), 7.66-7.28(22H, m), 6.75(1H, s), |
| | 6.69(2H, d), 6.58(1H, d), 6.39(1H, d), 1.72(6H, s) |
| 134 | δ = 7.91-7.89(2H, m), 7.81(1H, d), 7.66(2H, d), 7.54- |
| | 7.32(24H, m), 6.69(6H, d) |
| 136 | δ = 7.89(1H, d), 7.66(2H, d), 7.60(1H, d), 7.52-7.20(16H, |
| | m), 6.81(2H, t), 6.63(4H, d), 6.39(1H, d) |
| 160 | δ = 8.55(1H, d), 8.42(1H, d), 8.08-8.04(2H, m), 7.89- |
| | 7.87(2H, m), 7.66-7.55(8H, m), 7.43-7.11(17H, m), 6.81- |
| | 6.75(2H, m), 6.63-6.58(3H, m), 6.39(1H, d) |
| 168 | δ = 8.00(2H, d), 7.92-7.89(2H, m), 7.73(1H, d), 7.66- |
| | 7.32(23H, m), 6.69(4H, d), 6.39(1H, d) |
| 211 | δ = 7.89-7.81(4H, m), 7.66-7.60(4H, m), 7.43-7.32(6H, m), |
| | 7.20(4H, t), 6.81(2H, t), 6.63(4H, d), 6.39(1H, d) |
| 229 | δ = 8.45-8.41(2H, m), 8.20(1H, d), 7.98(1H, d), 7.89- |
| | 7.87(2H, m), 7.66-7.20(15H, m), 6.81(1H, t), 6.75(1H, s), |
| | 6.63-6.58(3H, m), 6.39(1H, d), 1.72(6H, s) |
| 243 | δ = 7.89(1H, d), 7.66-7.32(26H, m), 6.69(4H, d) |
| 247 | δ = 7.89-7.87(3H, m), 7.66-7.51(12H, m), 7.43-7.28(8H, m), |
| | 6.75(2H, s), 6.58(2H, d), 1.72(12H, s) |
| 255 | δ = 7.89(1H, d), 7.67-7.62(5H, m), 7.55-7.28(20H, m), |
| | 6.75(1H, s), 6.69(4H, d), 6.58(1H, d), 1.72(6H, s) |
| 257 | δ = 7.89(1H, d), 7.66-7.20(25H, m), 6.81(1H, t), 6.69(2H, |
| | d), 6.63(2, d) |
| 267 | δ = 7.89(1H, d), 7.67-7.63(4H, m), 7.54-7.20(18H, m), |
| | 6.81(2H, t), 6.69-6.63(6H, m) |
| 279 | δ = 8.55(1H, d), 8.42(1H, d), 8.08-8.02(4H, m), 7.89- |
| | 7.87(2H, m), 7.66-7.53(12H, m), 7.43-7.28(6H, m), 6.98(1H, |
| | d), 6.75(1H, s), 6.58(1H, d), 1.72(6H, s) |
| 292 | δ = 8.00(2H, d), 7.92-7.87(4H, m), 7.73(1H, d), 7.66- |
| | 7.55(11H, m), 7.43-7.28(7H, m), 6.75(2H, s), 6.58(2H, d), |
| | 1.72(12H, s) |
| 299 | δ = 8.00(2H, d), 7.92-7.89(2H, m), 7.66-7.32(27H, m), |
| | 6.69(6H, d) |
| 311 | δ = 8.93(2H, d), 8.12(2H, d), 7.93-7.75(9H, m), 7.66- |
| | 7.16(19H, m), 6.81(1H, t), 6.63(2H, d), 6.55(1H, s), |
| | 6.39(1H, d) |
| 334 | δ = 7.89-7.81(5H, m), 7.66-7.55(7H, m), 7.43-7.20(10H, m), |
| | 6.81-6.75(2H, m), 6.63-6.58(3H, m), 1.72(6H, s) |
| 347 | δ = 8.45-8.41(2H, m), 8.20(1H, d), 7.98(1H, d), 7.89(1H, |
| | d), 7.66-7.32(17H, m), 7.20(2H, t), 6.81(1H, t), 6.69- |
| | 6.63(4H, m) |
| 363 | δ = 7.89(1H, d), 7.66(2H, d), 7.57-7.32(23H, m), 7.07(1H, |
| | s), 6.69(4H, d) |
| 365 | δ = 7.89-7.87(2H, m), 7.66-7.28(22H, m), 7.07(1H, s), |
| | 6.75(1H, s), 6.69(2H, d), 6.58(1H, d), 1.72(6H, s) |
| 371 | δ = 7.89-7.87(3H, m), 7.75(1H, d), 7.66(2H, d), 7.52- |
| | 7.16(21H, m), 7.07(1H, s), 6.81(1H, t), 6.63(2H, d), |
| | 6.55(1H, s), 6.39(1H, d) |
| 375 | δ = 7.89-7.87(2H, m), 7.67-7.62(5H, m), 7.55-7.28(20H, m), |
| | 6.75(1H, s), 6.69(4H, d), 6.58(1H, d), 1.72(6H, s) |
| 378 | δ = 7.89(1H, d), 7.66(2H, d), 7.57-7.32(23H, m), 7.25(4H, |
| | s), 7.07(1H, s), 6.69(4H, d) |
| 395 | δ = 8.55(1H, d), 8.42(1H, d), 8.08(1H, d), 8.04(1H, d), |
| | 7.89-7.87(2H, m), 7.66-7.38(20H, m), 7.07(1H, s), 6.75(1H, |
| | s), 6.69(2H, d), 6.58(1H, d), 1.72(6H, s) |
| 411 | δ = 8.00(2H, d), 7.92-7.87(3H, m), 7.73-7.28(18H, m), 7.16- |
| | 7.07(4H, m), 6.87(1H, t), 6.75(1H, s), 6.69(1H, d), |
| | 6.58(1H, d), 1.72(6H, s) |
| 457 | δ = 7.89-7.81(6H, m), 7.66-7.55(8H, m), 7.43-7.28(10H, m), |
| | 7.07(1H, s), 6.75(1H, s), 6.58(2H, d), 1.72(12H, s) |
| 603 | δ = 7.93-7.87(3H, m), 7.77(1H, s), 7.69-7.66(3H, m), 7.55- |
| | 7.31(22H, m), 6.69(4H, d), 1.72(6H, s) |
| 605 | δ = 7.93-7.87(4H, m), 7.77(1H, s), 7.66-7.62(4H, m), 7.55- |
| | 7.28(18H, m), 6.75(1H, s), 6.69(2H, d), 6.58(1H, d), |
| | 1.72(12H, s) |
| 632 | δ = 7.93-7.87(3H, m), 7.77(1H, s), 7.66-7.20(20H, m) |
| | 6.81(1H, t), 6.69(2H, d), 6.63(2H, d), 1.72(6H, s) |
| 651 | δ = 7.93-7.87(4H, m), 7.77(1H, s), 7.66-7.51(10H, m), 7.43- |
| | 7.28(8H, m), 7.16(1H, t), 7.08-7.07(3H, m), 6.87(1H, t), |
| | 6.75(1H, s), 6.69(1H, d), 6.58(1H, d), 1.72(12H, s) |
| 657 | δ = 7.93-7.87(3H, m), 7.77(1H, s), 7.67-7.63(5H, m), 7.55- |
| | 7.54(3H, m), 7.43-7.20(9H, m), 6.81(2H, t), 6.69-6.63(6H, |
| | m), 1.72(6H, s) |

TABLE 4

| Compound | FD-MS | Compound | FD-MS |
|---|---|---|---|
| 002 | m/z = | 005 | m/z = |
| | 577.67(C₄₂H₂₇NO₂ = 577.20) | | 693.83(C₅₁H₃₅NO₂ = 693.27) |
| 012 | m/z = | 015 | m/z = |
| | 577.67(C₄₂H₂₇NO₂ = 577.20) | | 769.93(C₅₇H₃₉NO₂ = 769.30) |
| 019 | m/z = | 033 | m/z = |
| | 693.83(C₅₁H₃₅NO₂ = 693.27) | | 703.82(C₅₂H₃₃NO₂ = 703.25) |
| 052 | m/z = | 080 | m/z = |
| | 783.95(C₅₈H₄₁NO₂ = 783.31) | | 844.01(C₆₃H₄₁NO₂ = 843.31) |
| 098 | m/z = | 106 | m/z = |
| | 717.81(C₅₂H₃₁NO₃ = 717.23) | | 607.62(C₄₂H₂₅NO₂S = 607.16) |
| 123 | m/z = | 125 | m/z = |
| | 653.77(C₄₈H₃₁NO₂ = 653.24) | | 693.83(C₅₁H₃₅NO₂ = 693.27) |
| 134 | m/z = | 136 | m/z = |
| | 729.86(C₅₄H₃₅NO₂ = 729.27) | | 577.67(C₄₂H₂₇NO₂ = 577.20) |
| 160 | m/z = | 168 | m/z = |
| | 791.93(C₅₉H₃₇NO₂ = 791.28) | | 703.82(C₅₂H₃₃NO₂ = 703.25) |
| 211 | m/z = | 229 | m/z = |
| | 591.65(C₄₂H₂₅NO₃ = 591.18) | | 723.88(C₅₁H₃₃NO₂S = 723.22) |
| 243 | m/z = | 247 | m/z = |
| | 653.77(C₄₈H₃₁NO₂ = 653.24) | | 733.89(C₅₄H₃₉NO₂ = 733.30) |
| 255 | m/z = | 257 | m/z = |
| | 769.93(C₅₇H₃₉NO₂ = 769.30) | | 653.77(C₄₈H₃₁NO₂ = 653.24) |
| 267 | m/z = | 279 | m/z = |
| | 653.77(C₄₈H₃₁NO₂ = 653.24) | | 717.85(C₅₃H₃₅NO₂ = 717.27) |
| 292 | m/z = | 299 | m/z = |
| | 783.95(C₅₈H₄₁NO₂ = 783.31) | | 779.92(C₅₈H₃₇NO₂ = 779.28) |
| 311 | m/z = | 334 | m/z = |
| | 839.97(C₆₃H₃₇NO₂ = 839.28) | | 707.81(C₅₁H₃₃NO₃ = 707.25) |
| 347 | m/z = | 363 | m/z = |
| | 683.81(C₄₈H₂₉NO₂S = 683.19) | | 653.77(C₄₈H₃₁NO₂ = 653.24) |
| 365 | m/z = | 371 | m/z = |
| | 693.83(C₅₁H₃₅NO₂ = 693.27) | | 739.86(C₅₅H₃₃NO₂ = 739.25) |
| 375 | m/z = | 378 | m/z = |
| | 769.93(C₅₇H₃₉NO₂ = 769.30) | | 729.86(C₅₄H₃₅NO₂ = 729.27) |
| 395 | m/z = | 411 | m/z = |
| | 743.89(C₅₅H₃₇NO₂ = 743.28) | | 743.89(C₅₅H₃₇NO₂ = 743.28) |
| 457 | m/z = | 603 | m/z = |
| | 823.97(C₆₀H₄₁NO₃ = 823.31) | | 769.93(C₅₇H₃₉NO₂ = 769.30) |
| 605 | m/z = | 632 | m/z = |
| | 809.99(C₆₀H₄₃NO₂ = 809.33) | | 693.83(C₅₁H₃₅NO₂ = 693.27) |
| 651 | m/z = | 657 | m/z = |
| | 809.99(C₆₀H₄₃NO₂ = 809.33) | | 693.83(C₅₁H₃₅NO₂ = 693.27) |

### EXPERIMENTAL EXAMPLES

### Experimental Example 1

(1) Manufacture of Organic Light-Emitting Device

A glass substrate coated with a thin film of ITO to a thickness of 1500 Å was washed with distilled water ultrasonic waves. After finishing the distilled water, it was ultrasonically washed with a solvent such as acetone, methanol, isopropyl alcohol, and the like, and dried, and then UVO treatment was performed for 5 minutes using UV in a UV washer. Next, the substrate was transferred to a plasma cleaner (PT), and then plasma-treated for the ITO work function and residual film removal in a vacuum state and transferred to a thermal deposition equipment for organic deposition.

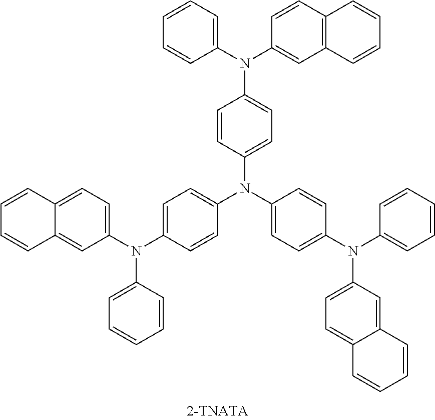 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Next, after evacuating the chamber until the vacuum degree reached 10⁻⁶ torr, an electric current was applied to the cell to evaporate 2-TNATA, thereby depositing a 600 Å-thick hole injection layer on the ITO substrate. The following N,N′-bis(a-naphthyl)-N,N′-diphenyl-4,4′-diamine (NPB) was placed in another cell in the vacuum deposition equipment and evaporated by applying an electric current to the cell, thereby depositing a 300 Å-thick hole transport layer on the hole injection layer.

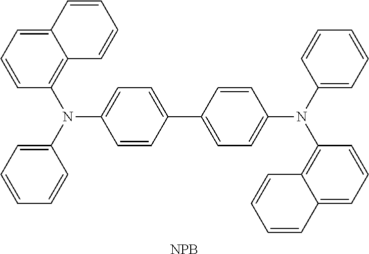 [SMILES: c1ccc(N(c2ccc(-c3ccc(N(c4ccccc4)c4cccc5ccccc45)cc3)cc2)c2cccc3ccccc23)cc1 ; "NPB"]

A light-emitting layer was thermally vacuum deposited thereon as follows. In the light emitting layer, compound 9-[4-(4,6-diphenyl-1,3,5-triazin-2-yl)phenyl]-9′-phenyl-3,3′-Bi-9H-carbazole was deposited at 400 Å as a host, and the green phosphorescent dopant was deposited by doping Ir(ppy)₃ at 7%. Next, BCP was deposited at 60 Å as a hole-blocking layer, and Alq₃ was deposited at 200 Å thereon as an electron transport layer. Finally, an electron injection layer was formed by depositing lithium fluoride (LiF) on the electron transport layer to a thickness of 10 Å, and then a cathode was formed by depositing an aluminum (Al) cathode to a thickness of 1,200 Å on the electron injection layer, thereby manufacturing a light-emitting device.

On the other hand, all organic compounds required for manufacturing OLED devices were vacuum sublimated and purified under 10⁻⁶ to 10⁻⁸ torr for each material before use in OLED manufacturing.

An organic light-emitting device was prepared in the same manner as in Experimental Example 1, except that the compound of the present invention described in Table 5 below was used instead of NPB, which is a compound used in forming the hole transport layer in Experimental Example 1, and the driving voltage and luminous efficiency of the organic light-emitting device were measured and the results are shown in Table 5 below.

In this case, the following compounds were used as hole transport compounds of comparative examples except for NPB.

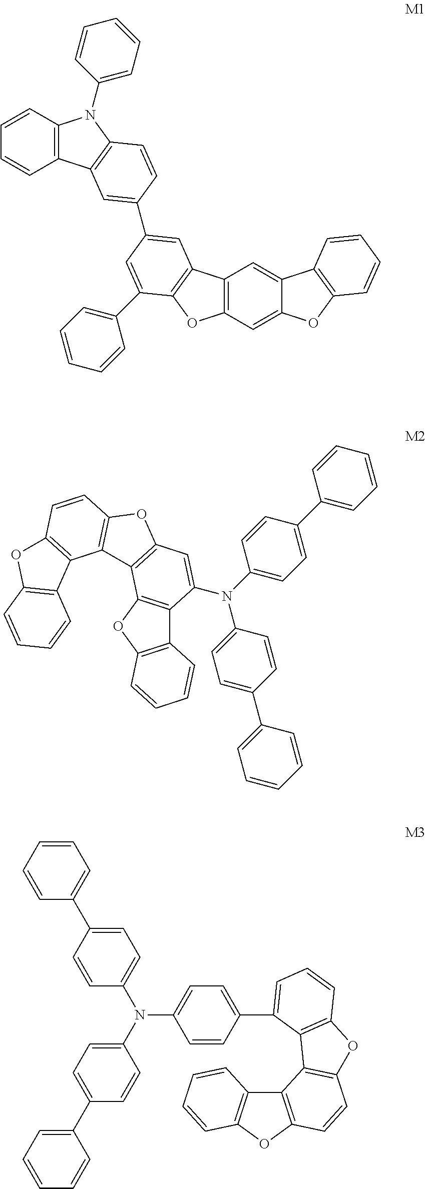 [SMILES: c1ccc(-c2cc(-c3ccc4c(c3)c3ccccc3n4-c3ccccc3)cc3c2oc2cc4oc5ccccc5c4cc23)cc1 ; c1ccc(-c2ccc(N(c3ccc(-c4ccccc4)cc3)c3cc4oc5ccc6oc7ccccc7c6c5c4c4oc5ccccc5c34)cc2)cc1 ; c1ccc(-c2ccc(N(c3ccc(-c4ccccc4)cc3)c3ccc(-c4cccc5oc6ccc7oc8ccccc8c7c6c45)cc3)cc2)cc1]

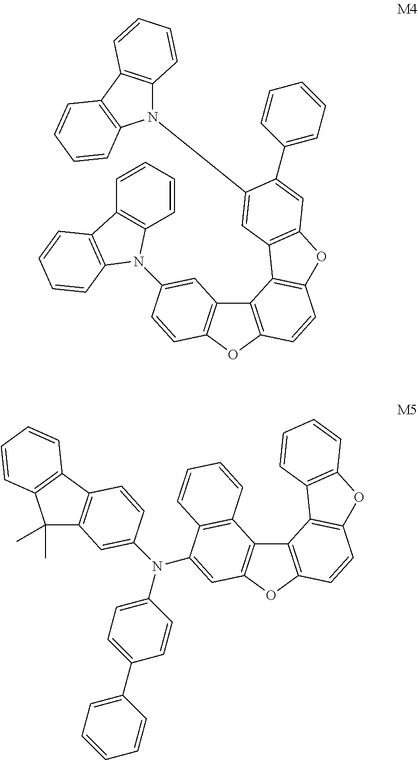 [SMILES: c1ccc(-c2cc3oc4ccc5oc6ccc(-n7c8ccccc8c8ccccc87)cc6c5c4c3cc2-n2c3ccccc3c3ccccc32)cc1 ; CC1(C)c2ccccc2-c2ccc(N(c3ccc(-c4ccccc4)cc3)c3cc4oc5ccc6oc7ccccc7c6c5c4c4ccccc34)cc21]

(2) Driving Voltage and Luminous Efficiency of Organic Light-Emitting Device

For the organic light-emitting device manufactured as described above, electroluminescence (EL) properties were measured with M7000 from McScience, and based on the measured results, T₉₅ was measured when the reference luminance was 6,000 cd/m² through the lifespan measuring device (M6000) manufactured by McScience.

The properties of the organic light-emitting device of the present invention are shown in Table 5 below.

TABLE 5

| | | Driving | Luminous | |
|---|---|---|---|---|
| | | Voltage | Efficiency | lifespan |
| | Compound | (V) | (cd/A) | (T95) |
| Working | 002 | 4.09 | 118.32 | 144 |
| Example 1 | | | | |
| Working | 005 | 3.82 | 125.36 | 169 |
| Example 2 | | | | |
| Working | 012 | 3.96 | 117.55 | 145 |
| Example 3 | | | | |
| Working | 016 | 4.16 | 121.39 | 136 |
| Example 4 | | | | |
| Working | 019 | 4.01 | 121.12 | 174 |
| Example 5 | | | | |
| Working | 033 | 4.06 | 120.54 | 172 |
| Example 6 | | | | |
| Working | 052 | 4.12 | 118.52 | 142 |
| Example 7 | | | | |
| Working | 080 | 4.00 | 113.54 | 133 |
| Example 8 | | | | |
| Working | 098 | 3.96 | 120.51 | 159 |
| Example 9 | | | | |
| Working | 106 | 3.89 | 120.23 | 145 |
| Example 10 | | | | |
| Working | 123 | 3.79 | 125.90 | 172 |
| Example 11 | | | | |
| Working | 125 | 4.04 | 120.76 | 141 |
| Example 12 | | | | |
| Working | 134 | 4.09 | 121.22 | 158 |
| Example 13 | | | | |
| Working | 136 | 4.05 | 120.66 | 135 |
| Example 14 | | | | |
| Working | 160 | 4.18 | 120.17 | 187 |
| Example 15 | | | | |
| Working | 168 | 4.05 | 121.74 | 144 |
| Example 16 | | | | |
| Working | 211 | 4.02 | 119.53 | 153 |
| Example 17 | | | | |
| Working | 229 | 3.83 | 125.57 | 181 |
| Example 18 | | | | |
| Working | 243 | 3.97 | 118.78 | 151 |
| Example 19 | | | | |
| Working | 247 | 4.10 | 117.98 | 138 |
| Example 20 | | | | |
| Working | 255 | 4.11 | 119.79 | 152 |
| Example 21 | | | | |
| Working | 257 | 4.04 | 118.26 | 145 |
| Example 22 | | | | |
| Working | 267 | 3.97 | 117.74 | 160 |
| Example 23 | | | | |
| Working | 279 | 4.22 | 118.41 | 152 |
| Example 24 | | | | |
| Working | 292 | 4.17 | 119.64 | 161 |
| Example 25 | | | | |
| Working | 299 | 4.17 | 120.33 | 157 |
| Example 26 | | | | |
| Working | 311 | 4.09 | 121.46 | 140 |
| Example 27 | | | | |
| Working | 334 | 3.98 | 120.33 | 162 |
| Example 28 | | | | |
| Working | 347 | 3.78 | 125.82 | 177 |
| Example 29 | | | | |
| Working | 363 | 4.11 | 121.09 | 145 |
| Example 30 | | | | |
| Working | 365 | 4.14 | 120.11 | 144 |
| Example 31 | | | | |
| Working | 371 | 4.10 | 117.89 | 147 |
| Example 32 | | | | |
| Working | 375 | 4.08 | 120.54 | 154 |
| Example 33 | | | | |
| Working | 378 | 4.13 | 120.32 | 147 |
| Example 34 | | | | |
| Working | 395 | 4.06 | 119.89 | 173 |
| Example 35 | | | | |
| Working | 411 | 4.15 | 121.30 | 177 |
| Example 36 | | | | |
| Working | 457 | 3.92 | 120.96 | 155 |
| Example 37 | | | | |
| Working | 603 | 4.00 | 116.47 | 141 |
| Example 38 | | | | |
| Working | 605 | 4.08 | 119.22 | 151 |
| Example 39 | | | | |
| Working | 632 | 4.05 | 121.17 | 145 |
| Example 40 | | | | |
| Working | 651 | 4.11 | 120.41 | 139 |
| Example 41 | | | | |
| Working | 657 | 4.18 | 120.76 | 169 |
| Example 42 | | | | |
| Comparative | NPB | 4.61 | 99.47 | 113 |
| Example 1 | | | | |
| Comparative | M1 | 4.38 | 108.55 | 120 |
| Example 2 | | | | |
| Comparative | M2 | 4.37 | 107.86 | 128 |
| Example 3 | | | | |
| Comparative | M3 | 4.41 | 105.82 | 119 |
| Example 4 | | | | |
| Comparative | M4 | 4.39 | 104.24 | 122 |
| Example 5 | | | | |
| Comparative | M5 | 4.37 | 108.13 | 107 |
| Example 6 | | | | |

It could be seen that the devices of Examples 1 to 42 according to one embodiment of the present invention had a lower driving voltage and excellent efficiency and lifespan compared to the devices of Comparative Examples 1 to 6.

### Experimental Example 2

(1) Manufacture of Organic Light-Emitting Device

The transparent electrode ITO thin film obtained from the glass for OLED (manufactured by Samsung Corning) was subject to ultrasonic washing for each 5 minutes using trichloroethylene, acetone, ethanol, and distilled water sequentially, and then stored in isopropanol before use. Next, the ITO substrate was installed in the substrate folder of the vacuum deposition equipment, and the following 4,4′,4″-tris(N,N-(2-naphthyl)-phenylamino)triphenylamine (2-TNATA) was placed in the cell in the vacuum deposition equipment.

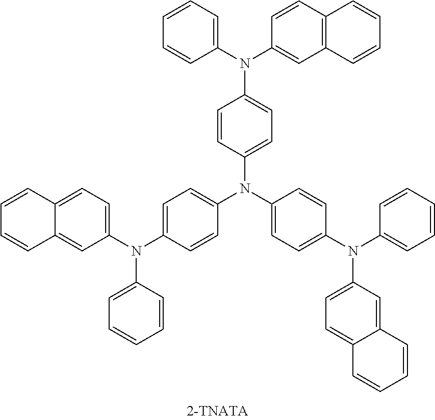 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Next, after evacuating the chamber until the vacuum degree reached 10⁻⁶ torr, an electric current was applied to the cell to evaporate 2-TNATA, thereby depositing a 600 Å-thick hole injection layer on the ITO substrate. The following N,N′-bis(a-naphthyl)-N,N′-diphenyl-4,4′-diamine (NPB) was placed in another cell in the vacuum deposition equipment and evaporated by applying an electric current to the cell, thereby depositing a 300 Å-thick hole transport layer on the hole injection layer.

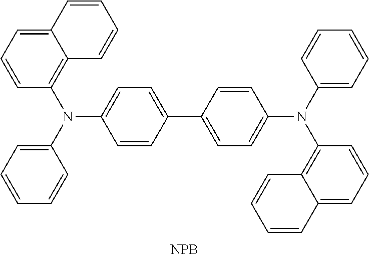 [SMILES: c1ccc(N(c2ccc(-c3ccc(N(c4ccccc4)c4cccc5ccccc45)cc3)cc2)c2cccc3ccccc23)cc1 ; "NPB"]

After the hole injection layer and the hole transport layer were formed in this way, a blue light-emitting material having the following structure was deposited as a light-emitting layer thereon. Specifically, the blue light-emitting host material H1 was vacuum-deposited to a thickness of 200 Å in one cell in the vacuum deposition equipment, and the blue light-emitting dopant material D1 was vacuum-deposited at 5% thereon compared to the host material.

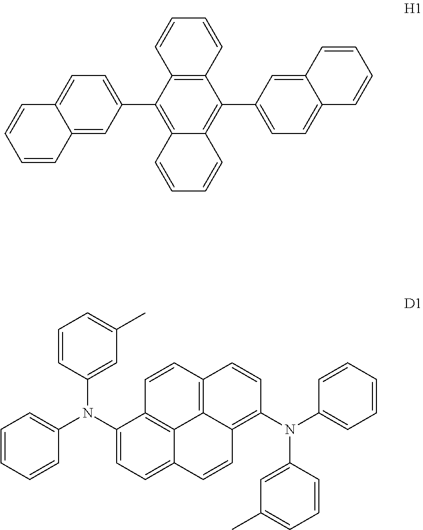

Next, an electron transport layer was deposited to a thickness of 300 Å with the compounds of Structure Formula E1 below.

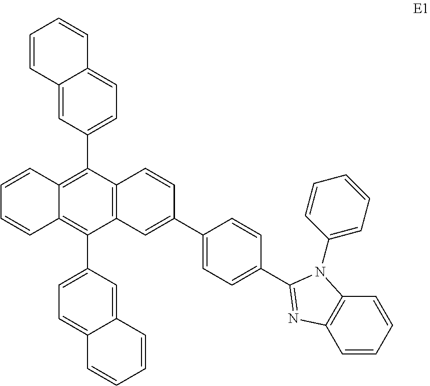 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An electron injection layer was deposited to a thickness of 10 Å with lithium fluoride (LiF) and an Al cathode was deposited to a thickness of 1,000 Å, thereby manufacturing an OLED device. On the other hand, all organic compounds required for manufacturing OLED devices were vacuum sublimated and purified under 10⁻⁶ to 10⁻⁸ torr for each material before use in OLED manufacturing.

An organic light-emitting device was manufactured in the same manner as in Experimental Example 2 above, except that the thickness of the hole transport layer NPB in Experimental Example 2 above was formed at 150 Å, and then an electron-blocking layer was further formed to a thickness of 50 Å with the compound of the present invention described in Table 6 below on the hole transport layer. The driving voltage, luminous efficiency, and lifespan of the manufactured blue organic light-emitting device were measured, and the results are shown in Table 6 below.

In this case, the electron-blocking layer compound used as a comparative example is as follows.

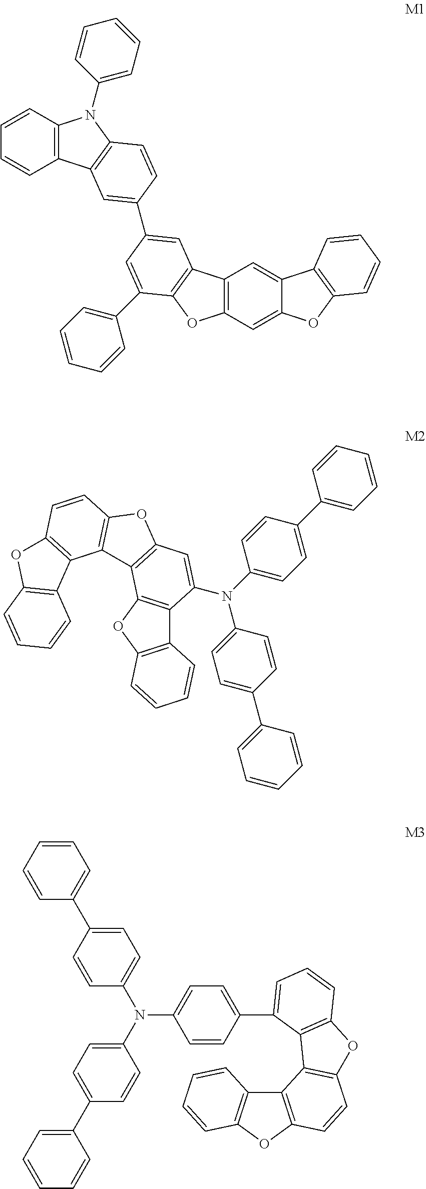 [SMILES: c1ccc(-c2cc(-c3ccc4c(c3)c3ccccc3n4-c3ccccc3)cc3c2oc2cc4oc5ccccc5c4cc23)cc1 ; c1ccc(-c2ccc(N(c3ccc(-c4ccccc4)cc3)c3cc4oc5ccc6oc7ccccc7c6c5c4c4oc5ccccc5c34)cc2)cc1 ; c1ccc(-c2ccc(N(c3ccc(-c4ccccc4)cc3)c3ccc(-c4cccc5oc6ccc7oc8ccccc8c7c6c45)cc3)cc2)cc1]

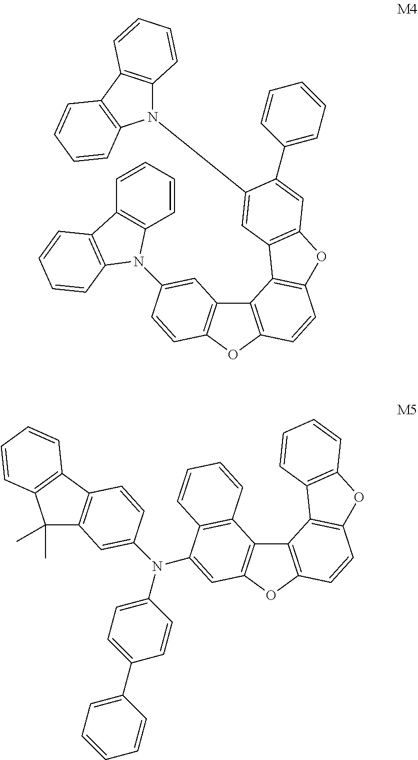 [SMILES: c1ccc(-c2cc3oc4ccc5oc6ccc(-n7c8ccccc8c8ccccc87)cc6c5c4c3cc2-n2c3ccccc3c3ccccc32)cc1 ; CC1(C)c2ccccc2-c2ccc(N(c3ccc(-c4ccccc4)cc3)c3cc4oc5ccc6oc7ccccc7c6c5c4c4ccccc34)cc21]

TABLE 6

| | | Driving | Luminous | |
|---|---|---|---|---|
| | | Voltage | Efficiency | lifespan |
| | Compound | (V) | (cd/A) | (T95) |
| Working | 005 | 5.20 | 7.21 | 62 |
| Example 43 | | | | |
| Working | 080 | 5.31 | 7.59 | 67 |
| Example 44 | | | | |
| Working | 125 | 5.22 | 7.26 | 61 |
| Example 45 | | | | |
| Working | 136 | 5.36 | 6.97 | 59 |
| Example 46 | | | | |
| Working | 247 | 5.15 | 7.23 | 68 |
| Example 47 | | | | |
| Working | 257 | 5.36 | 6.94 | 71 |
| Example 48 | | | | |
| Working | 334 | 5.29 | 7.33 | 69 |
| Example 49 | | | | |
| Working | 365 | 5.30 | 6.98 | 72 |
| Example 50 | | | | |
| Working | 395 | 5.25 | 7.39 | 56 |
| Example 51 | | | | |
| Working | 457 | 5.31 | 7.16 | 70 |
| Example 52 | | | | |
| Working | 632 | 5.36 | 6.91 | 68 |
| Example 53 | | | | |
| Working | 651 | 5.24 | 7.07 | 64 |
| Example 54 | | | | |
| Comparative | M1 | 5.59 | 6.78 | 52 |
| Example 7 | | | | |
| Comparative | M2 | 5.53 | 6.61 | 55 |
| Example 8 | | | | |
| Comparative | M3 | 5.51 | 6.44 | 50 |
| Example 9 | | | | |
| Comparative | M4 | 5.65 | 6.57 | 51 |
| Example 10 | | | | |
| Comparative | M5 | 5.59 | 6.40 | 53 |
| Example 11 | | | | |

### Experimental Example 3

1) Manufacture of Organic Light-Emitting Device

A glass substrate coated with a thin film of indium tin oxide (ITO) to a thickness of 1500 Å was washed with distilled water ultrasonic waves. After finishing the distilled water, it was ultrasonically washed with a solvent such as acetone, methanol, isopropyl alcohol, and the like, and dried, and then UVO treatment was performed for 5 minutes using UV in a UV washer. Next, the substrate was transferred to a plasma cleaner (PT), and then plasma-treated for the ITO work function and residual film removal in a vacuum state and transferred to a thermal deposition equipment for organic deposition.

The hole injection layer 2-TNATA (4,4′,4″-tris[2-naphthyl(phenyl)amino]triphenylamine) and the hole transport layer NPB (N,N′-di(1-naphthyl)-N,N′-diphenyl-(1,1′-biphenyl)-4,4′-diamine) were formed as a common layer on the ITO transparent electrode (anode).

A light-emitting layer was thermally vacuum deposited thereon as follows. The light-emitting layer was deposited to a thickness of 500 Å by using a method of depositing two host compounds from one source using the compound described in Table 7 below as a host, n-Host (n-type host) with good electron transport ability as a single host or a first host, and p-Host (p-type host) with good hole transport ability as a second host, and doping (piq)₂(Ir) (acac) at 3% based on the weight of the host material using (piq)₂(Ir) (acac) as a red phosphorescent dopant in the host, or doping Ir(ppy)₃ at 7% based on the weight of the host material using Ir(ppy)₃ as a green phosphorescent dopant in the host.

Next, BCP was deposited to a thickness of 60 Å as a hole-blocking layer, and Alq₃ was deposited to a thickness of 200 Å thereon as an electron transport layer.

In this case, when two hosts are used, the compound used as the n-Host (first host) is as follows.

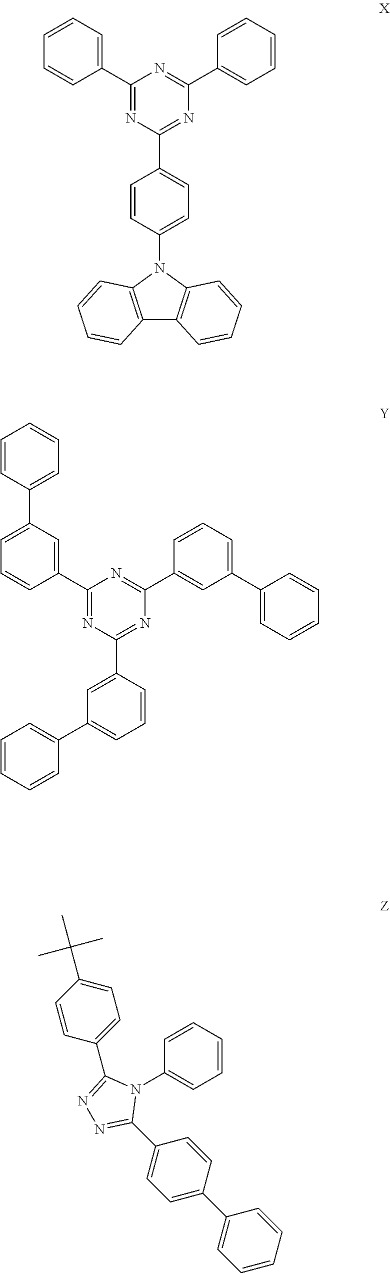 [SMILES: c1ccc(-c2nc(-c3ccccc3)nc(-c3ccc(-n4c5ccccc5c5ccccc54)cc3)n2)cc1 ; c1ccc(-c2cccc(-c3nc(-c4cccc(-c5ccccc5)c4)nc(-c4cccc(-c5ccccc5)c4)n3)c2)cc1 ; CC(C)(C)c1ccc(-c2nnc(-c3ccc(-c4ccccc4)cc3)n2-c2ccccc2)cc1]

Finally, an electron injection layer was formed by depositing lithium fluoride (LiF) on the electron transport layer to a thickness of 10 Å, and then a cathode was formed by depositing an aluminum (Al) cathode to a thickness of 1,200 Å on the electron injection layer, thereby manufacturing a light-emitting device.

Specifically, the compounds used as hosts in Examples 55 to 79 and Comparative Examples 12 to 21 are shown in Table 7 below.

In this case, the compounds M1 to M5 used as hosts in Comparative Examples 12 to 21 of Table 7 below are as follows.

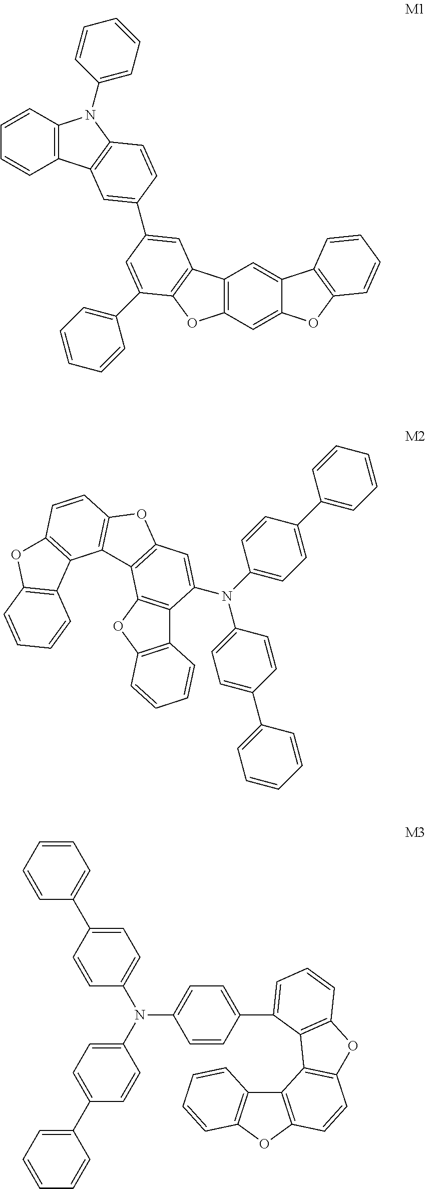 [SMILES: c1ccc(-c2cc(-c3ccc4c(c3)c3ccccc3n4-c3ccccc3)cc3c2oc2cc4oc5ccccc5c4cc23)cc1 ; c1ccc(-c2ccc(N(c3ccc(-c4ccccc4)cc3)c3cc4oc5ccc6oc7ccccc7c6c5c4c4oc5ccccc5c34)cc2)cc1 ; c1ccc(-c2ccc(N(c3ccc(-c4ccccc4)cc3)c3ccc(-c4cccc5oc6ccc7oc8ccccc8c7c6c45)cc3)cc2)cc1]

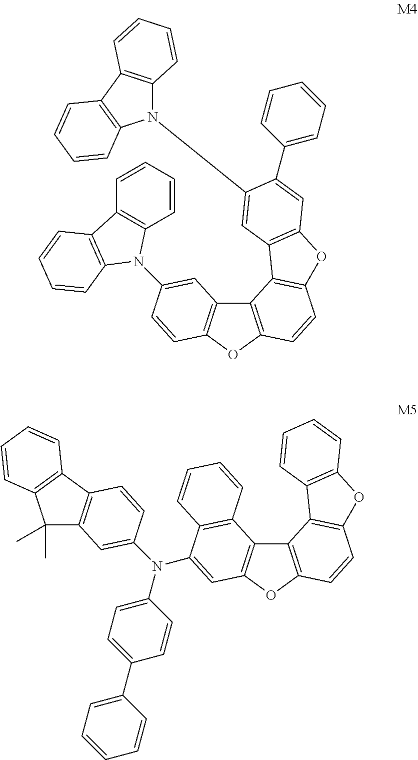 [SMILES: c1ccc(-c2cc3oc4ccc5oc6ccc(-n7c8ccccc8c8ccccc87)cc6c5c4c3cc2-n2c3ccccc3c3ccccc32)cc1 ; CC1(C)c2ccccc2-c2ccc(N(c3ccc(-c4ccccc4)cc3)c3cc4oc5ccc6oc7ccccc7c6c5c4c4ccccc34)cc21]

On the other hand, all organic compounds required for manufacturing organic light-emitting devices were vacuum sublimated and purified under 10⁻⁶ to 10⁻⁸ torr for each material before use in organic light-emitting device manufacturing.

2) Driving Voltage and Luminou Efficiency of Organic Light-Emitting Device

For the organic light-emitting device manufactured as described above, electroluminescence (EL) properties were measured with M7000 from McScience, and based on the measured results, T₉₅ was measured when the reference luminance was 6,000 cd/m² through the lifespan measuring device (M6000) manufactured by McScience. The measured results of the driving voltage, luminous efficiency, luminous color, and lifespan of the organic light-emitting device manufactured above are shown in Table 7 below.

TABLE 7

| | | | Driving | | | |
|---|---|---|---|---|---|---|
| | First | Second | Voltage | Efficiency | Luminous | lifespan |
| | Host | Host | (V) | (cd/A) | Color | (T₉₅) |
| Working | 015 | | 4.05 | 32.6 | red | 108 |
| Example 55 | | | | | | |
| Working | | | 3.85 | 68.2 | green | 81 |
| Example 56 | | | | | | |
| Working | 134 | | 4.04 | 31.9 | red | 105 |
| Example 57 | | | | | | |
| Working | | | 4.06 | 72.1 | green | 85 |
| Example 58 | | | | | | |
| Working | 243 | | 3.87 | 33.2 | red | 101 |
| Example 59 | | | | | | |
| Working | | | 3.97 | 67.8 | green | 92 |
| Example 60 | | | | | | |
| Working | 363 | | 3.79 | 35.2 | red | 114 |
| Example 61 | | | | | | |
| Working | | | 3.87 | 73.7 | green | 92 |
| Example 62 | | | | | | |
| Working | 378 | | 3.89 | 36.6 | red | 111 |
| Example 63 | | | | | | |
| Working | | | 3.97 | 71.4 | green | 93 |
| Example 64 | | | | | | |
| Working | X | 015 | 3.74 | 46.1 | red | 163 |
| Example 65 | | | | | | |
| Working | | 134 | 3.76 | 48.6 | red | 171 |
| Example 66 | | | | | | |
| Working | | 243 | 3.71 | 53.2 | red | 159 |
| Example 67 | | | | | | |
| Working | | 363 | 3.81 | 47.7 | red | 164 |
| Example 68 | | | | | | |
| Working | | 378 | 3.83 | 49.7 | red | 150 |
| Example 69 | | | | | | |
| Working | Y | 015 | 3.71 | 104.5 | green | 144 |
| Example 70 | | | | | | |
| Working | | 134 | 3.73 | 103.2 | green | 151 |
| Example 71 | | | | | | |
| Working | | 243 | 3.72 | 105.8 | green | 152 |
| Example 72 | | | | | | |
| Working | | 363 | 3.83 | 111.9 | green | 132 |
| Example 73 | | | | | | |
| Working | | 378 | 3.91 | 108.8 | green | 141 |
| Example 74 | | | | | | |
| Working | Z | 015 | 3.77 | 53.2 | red | 169 |
| Example 75 | | | | | | |
| Working | | 134 | 3.81 | 45.9 | red | 160 |
| Example 76 | | | | | | |
| Working | | 243 | 3.85 | 45.2 | red | 161 |
| Example 77 | | | | | | |
| Working | | 363 | 3.80 | 48.1 | red | 149 |
| Example 78 | | | | | | |
| Working | | 378 | 3.81 | 50.2 | red | 155 |
| Example 79 | | | | | | |
| Comparative | M1 | | 4.40 | 22.1 | red | 81 |
| Example 12 | | | | | | |
| Comparative | X | M1 | 4.38 | 42.5 | red | 132 |
| Example 13 | | | | | | |
| Comparative | M2 | | 4.64 | 62.8 | green | 65 |
| Example 14 | | | | | | |
| Comparative | Y | M2 | 4.45 | 85.9 | green | 112 |
| Example 15 | | | | | | |
| Comparative | M3 | | 4.42 | 22.8 | red | 79 |
| Example 16 | | | | | | |
| Comparative | X | M3 | 4.40 | 41.8 | red | 128 |
| Example 17 | | | | | | |
| Comparative | M4 | | 4.58 | 62.2 | green | 57 |
| Example 18 | | | | | | |
| Comparative | Y | M4 | 4.51 | 81.2 | green | 101 |
| Example 19 | | | | | | |
| Comparative | M5 | | 4.49 | 19.5 | red | 78 |
| Example 20 | | | | | | |
| Comparative | Z | M5 | 4.40 | 43.2 | red | 130 |
| Example 21 | | | | | | |

From Experimental Example 3 above, it could be confirmed that the organic light-emitting devices of Examples 55 to 64, in which the light emitting layer was formed by using the compound according to the present invention as a single host material, had excellent luminous efficiency and lifespan compared to the organic light-emitting devices of Comparative Examples 12, 14, 16, 18, and 20, which did not use the compound according to the present invention as a single host material.

From Experimental Example 3 above, it could be confirmed that the organic light-emitting devices of Examples 65 to 79, in which the light emitting layer was formed by using a first host material corresponding to n-Host and the compound according to the present invention as a second host material corresponding to p-Host at the same time, had excellent luminous efficiency and lifespan compared to the organic light-emitting devices of Comparative Examples 13, 15, 17, 19, and 21, in which the light-emitting layer was formed by using a first host material corresponding to n-Host and compounds other than the compound according to the present invention as a second host material corresponding to p-Host at the same time.

Such results mean that considering that an organic light-emitting device using n-Host (n-type host) having good electron transport ability as a first host and p-Host (p-type host) having good hole transporting ability as a second host is a single host material. in general, n-Host (n-type host) having good electron transport ability has superior luminous efficiency and lifespan than an organic light-emitting device using a single host material, the use of the compound according to the present invention as a host material may remarkably improve the luminous efficiency and lifespan of the organic light-emitting device.

This is judged to be because, when the compound according to the present invention is used as a host material, holes and electrons from each charge transport layer can be efficiently injected into the light-emitting layer. In addition, it is judged that such results are due to the influence by the orientation and space size formed by the interaction of materials during deposition.

In summary, since the efficient injection of holes and electrons into the light-emitting layer is also affected by the orientation and space size formed by the interaction of materials during deposition, the above results are judged to be due to the fact that the compounds of the present invention provide better effects in forming the orientation properties and space size than the M1 to M5 compounds.

The present invention is not limited to the above examples, but may be manufactured in a variety of different forms, and those of ordinary skill in the art to which the present invention pertains will understand that it may be implemented in other specific forms without changing the technical spirit or essential features of the present invention. Therefore, it should be understood that the examples described above are illustrative and not restrictive in all respects.

1. A heterocyclic compound represented by following Formula 1: 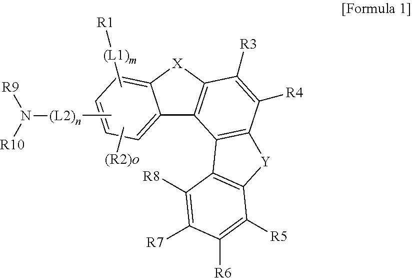
wherein:
X and Y are the same as or different from each other and each independently O or S;
R1 to R8 are the same as or different from each other and are each independently selected from the group consisting of hydrogen, deuterium, halogen, a cyano group, a substituted or unsubstituted C1 to C60 alkyl group, a substituted or unsubstituted C2 to C60 alkenyl group, a substituted or unsubstituted C2 to C60 alkynyl group, a substituted or unsubstituted C1 to C60 alkoxy group, a substituted or unsubstituted C3 to C60 cycloalkyl group, a substituted or unsubstituted C2 to C60 heterocycloalkyl group, a substituted or unsubstituted C6 to C60 aryl group, a substituted or unsubstituted C2 to C60 heteroaryl group, —P(═O)R101R102R103, and —NR101R102, wherein R101, R102, and R103 are the same as or different from each other and are each independently a substituted or unsubstituted C1 to C60 alkyl group, a substituted or unsubstituted C6 to C60 aryl group, or a substituted or unsubstituted C2 to C60 heteroaryl group; or two or more groups adjacent to each other combine with each other to form a substituted or unsubstituted C6 to C60 aromatic hydrocarbon ring or a substituted or unsubstituted C2 to C60 heterocycle;
R9 and R10 are the same as or different from each other and are each independently a substituted or unsubstituted C6 to C60 aryl group, or a substituted or unsubstituted C2 to C60 heteroaryl group;
L1 and L2 are the same as or different from each other and are each independently a direct bond, a substituted or unsubstituted C6 to C60 arylene group, or a substituted or unsubstituted C2 to C60 heteroarylene group;
m is an integer of 0 to 4, provided that when in is 0, L1 is a direct bond, and when m is 2 to 4, each L1 is the same as or different from each other and is each independently selected;
n is an integer of 0 to 4, provided that when n is 0, L2 is a direct bond, and when n is 2 to 4, each L2 is the same as or different from each other and is each independently selected;
o is an integer of 0 to 2, provided that when o is 2, each R2 is the same as or different from each other and is each independently selected.

2. The heterocyclic compound according to claim 1, characterized in that R1 is a substituted or unsubstituted C6 to C60 aryl group or a substituted or unsubstituted C2 to C60 heteroaryl group.

3. The heterocyclic compound according to claim 2, characterized in that R1, R9, and R10 are the same as or different from each other and are each independently substituted or unsubstituted phenyl, substituted or unsubstituted biphenyl, substituted or unsubstituted naphthalenyl, substituted or unsubstituted fluorenyl, substituted or unsubstituted 9,9-dimethylfluorenyl group, substituted or unsubstituted 9,9-diphenylfluorenyl group, substituted or unsubstituted spirobifluorenyl, substituted or unsubstituted phenanthrenyl, substituted or unsubstituted triphenylenyl, substituted or unsubstituted dibenzothiophenyl, or substituted or unsubstituted dibenzofuranyl group.

4. The heterocyclic compound according to claim 3, characterized in that R2 to R8 are the same as or different from each other and are each independently hydrogen or deuterium.

5. The heterocyclic compound according to claim 1, characterized in that
R1 is substituted or unsubstituted phenyl, substituted or unsubstituted biphenyl, substituted or unsubstituted naphthyl, substituted or unsubstituted phenanthrenyl, substituted or unsubstituted triphenylenyl, substituted or unsubstituted 9,9-dimethylfluorenyl, substituted or unsubstituted dibenzothiophenyl, or substituted or unsubstituted dibenzofuranyl;
R2 to R8 are the same as or different from each other and are each independently hydrogen or deuterium;
R9 and R10 are the same as or different from each other and are each independently substituted or unsubstituted phenyl, substituted or unsubstituted biphenyl, substituted or unsubstituted naphthyl, substituted or unsubstituted 9,9-dimethylfluorenyl, substituted or unsubstituted 9,9-diphenylfluorenyl, or substituted or unsubstituted spirobifluorenyl;
L1 and L2 are a direct bond or phenylene;
m and n are the same as or different from each other and are each independently an integer of 0 to 3; and
o is 2.

6. The heterocyclic compound according to claim 1, characterized in that the heterocyclic compound represented by Formula 1 is a compound represented by any one of the following compounds:

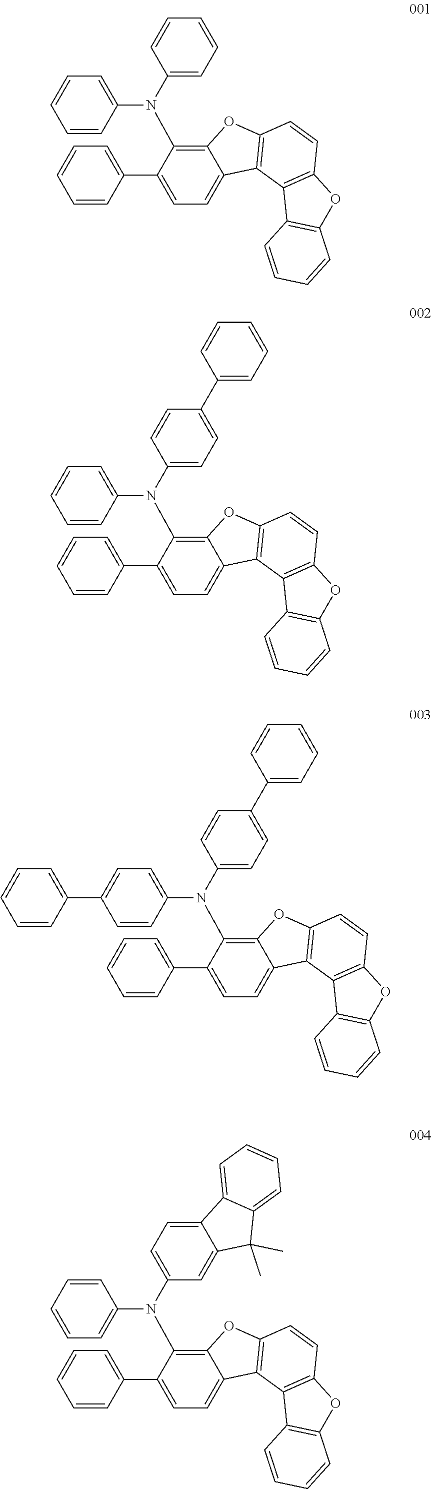 [SMILES: c1ccc(-c2ccc3c(oc4ccc5oc6ccccc6c5c43)c2N(c2ccccc2)c2ccccc2)cc1 ; c1ccc(-c2ccc(N(c3ccccc3)c3c(-c4ccccc4)ccc4c3oc3ccc5oc6ccccc6c5c34)cc2)cc1 ; c1ccc(-c2ccc(N(c3ccc(-c4ccccc4)cc3)c3c(-c4ccccc4)ccc4c3oc3ccc5oc6ccccc6c5c34)cc2)cc1 ; CC1(C)c2ccccc2-c2ccc(N(c3ccccc3)c3c(-c4ccccc4)ccc4c3oc3ccc5oc6ccccc6c5c34)cc21]

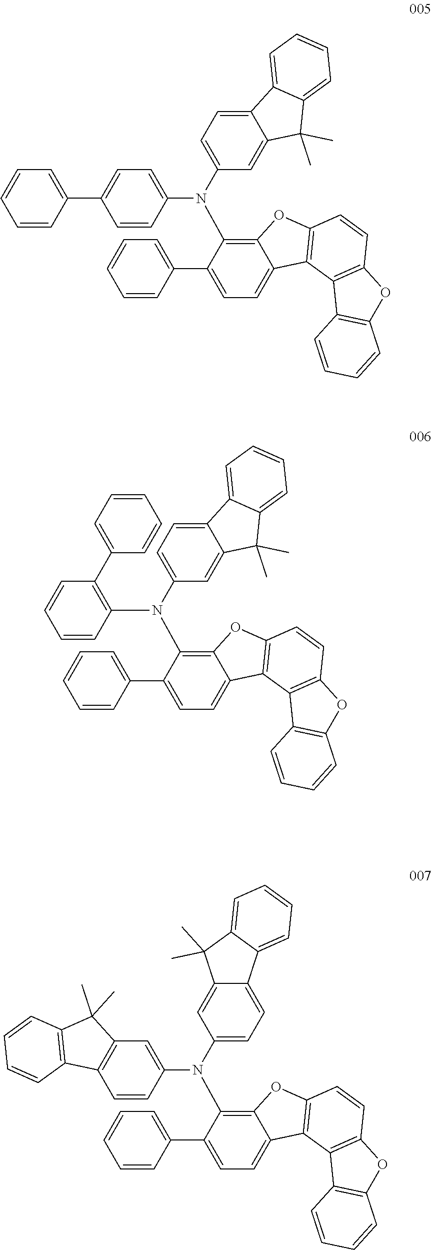 [SMILES: CC1(C)c2ccccc2-c2ccc(N(c3ccc(-c4ccccc4)cc3)c3c(-c4ccccc4)ccc4c3oc3ccc5oc6ccccc6c5c34)cc21 ; CC1(C)c2ccccc2-c2ccc(N(c3ccccc3-c3ccccc3)c3c(-c4ccccc4)ccc4c3oc3ccc5oc6ccccc6c5c34)cc21 ; CC1(C)c2ccccc2-c2ccc(N(c3ccc4c(c3)C(C)(C)c3ccccc3-4)c3c(-c4ccccc4)ccc4c3oc3ccc5oc6ccccc6c5c34)cc21]

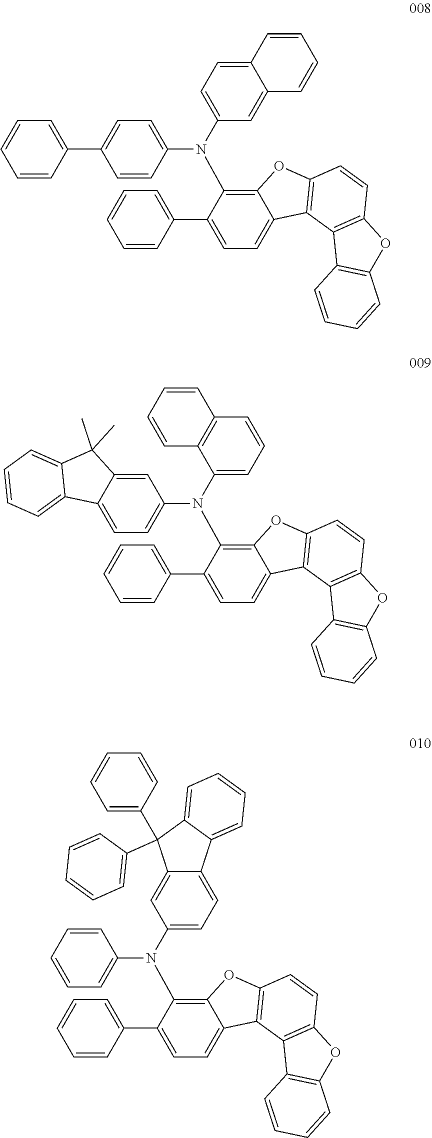 [SMILES: c1ccc(-c2ccc(N(c3ccc4ccccc4c3)c3c(-c4ccccc4)ccc4c3oc3ccc5oc6ccccc6c5c34)cc2)cc1 ; CC1(C)c2ccccc2-c2ccc(N(c3cccc4ccccc34)c3c(-c4ccccc4)ccc4c3oc3ccc5oc6ccccc6c5c34)cc21 ; c1ccc(-c2ccc3c(oc4ccc5oc6ccccc6c5c43)c2N(c2ccccc2)c2ccc3c(c2)C(c2ccccc2)(c2ccccc2)c2ccccc2-3)cc1]

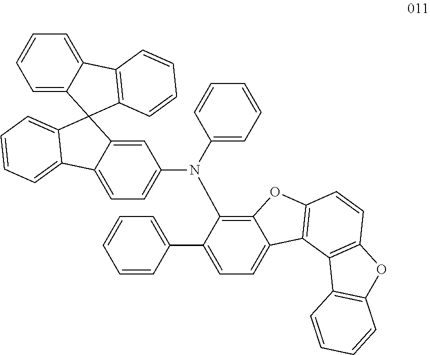 [SMILES: c1ccc(-c2ccc3c(oc4ccc5oc6ccccc6c5c43)c2N(c2ccccc2)c2ccc3c(c2)C2(c4ccccc4-c4ccccc42)c2ccccc2-3)cc1]

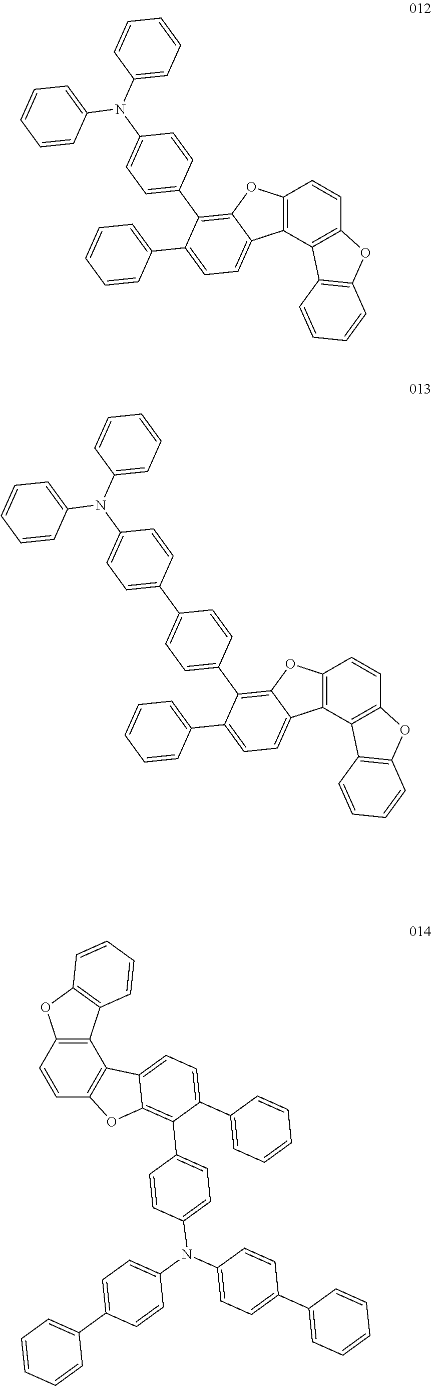 [SMILES: c1ccc(-c2ccc3c(oc4ccc5oc6ccccc6c5c43)c2-c2ccc(N(c3ccccc3)c3ccccc3)cc2)cc1 ; c1ccc(-c2ccc3c(oc4ccc5oc6ccccc6c5c43)c2-c2ccc(-c3ccc(N(c4ccccc4)c4ccccc4)cc3)cc2)cc1 ; c1ccc(-c2ccc(N(c3ccc(-c4ccccc4)cc3)c3ccc(-c4c(-c5ccccc5)ccc5c4oc4ccc6oc7ccccc7c6c45)cc3)cc2)cc1]

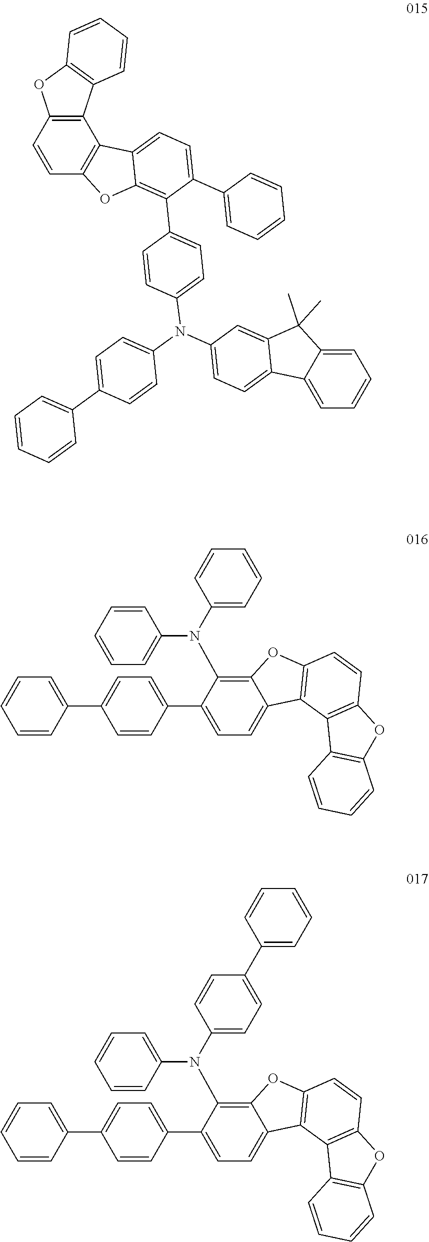 [SMILES: CC1(C)c2ccccc2-c2ccc(N(c3ccc(-c4ccccc4)cc3)c3ccc(-c4c(-c5ccccc5)ccc5c4oc4ccc6oc7ccccc7c6c45)cc3)cc21 ; c1ccc(-c2ccc(-c3ccc4c(oc5ccc6oc7ccccc7c6c54)c3N(c3ccccc3)c3ccccc3)cc2)cc1 ; c1ccc(-c2ccc(-c3ccc4c(oc5ccc6oc7ccccc7c6c54)c3N(c3ccccc3)c3ccc(-c4ccccc4)cc3)cc2)cc1]

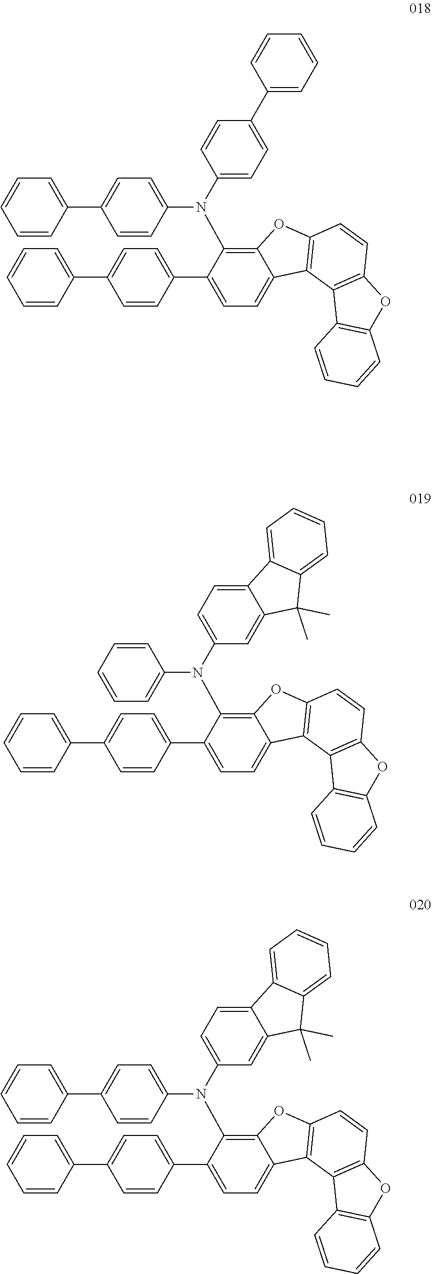 [SMILES: c1ccc(-c2ccc(-c3ccc4c(oc5ccc6oc7ccccc7c6c54)c3N(c3ccc(-c4ccccc4)cc3)c3ccc(-c4ccccc4)cc3)cc2)cc1 ; CC1(C)c2ccccc2-c2ccc(N(c3ccccc3)c3c(-c4ccc(-c5ccccc5)cc4)ccc4c3oc3ccc5oc6ccccc6c5c34)cc21 ; CC1(C)c2ccccc2-c2ccc(N(c3ccc(-c4ccccc4)cc3)c3c(-c4ccc(-c5ccccc5)cc4)ccc4c3oc3ccc5oc6ccccc6c5c34)cc21]

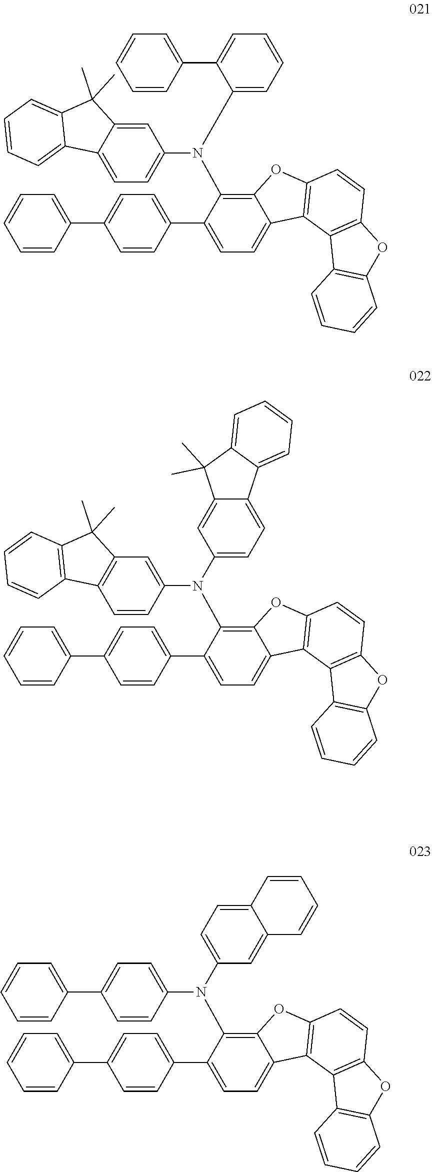 [SMILES: CC1(C)c2ccccc2-c2ccc(N(c3ccccc3-c3ccccc3)c3c(-c4ccc(-c5ccccc5)cc4)ccc4c3oc3ccc5oc6ccccc6c5c34)cc21 ; CC1(C)c2ccccc2-c2ccc(N(c3ccc4c(c3)C(C)(C)c3ccccc3-4)c3c(-c4ccc(-c5ccccc5)cc4)ccc4c3oc3ccc5oc6ccccc6c5c34)cc21 ; c1ccc(-c2ccc(-c3ccc4c(oc5ccc6oc7ccccc7c6c54)c3N(c3ccc(-c4ccccc4)cc3)c3ccc4ccccc4c3)cc2)cc1]

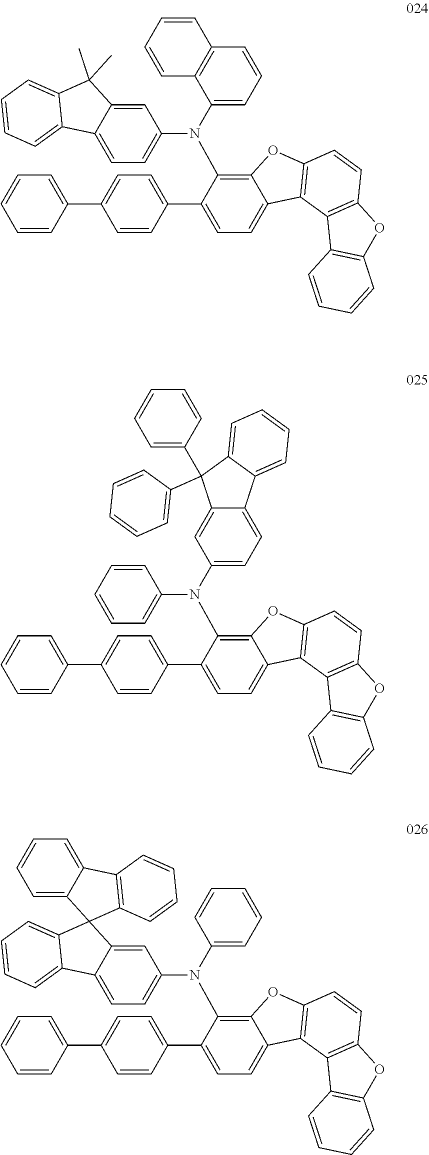 [SMILES: CC1(C)c2ccccc2-c2ccc(N(c3cccc4ccccc34)c3c(-c4ccc(-c5ccccc5)cc4)ccc4c3oc3ccc5oc6ccccc6c5c34)cc21 ; c1ccc(-c2ccc(-c3ccc4c(oc5ccc6oc7ccccc7c6c54)c3N(c3ccccc3)c3ccc4c(c3)C(c3ccccc3)(c3ccccc3)c3ccccc3-4)cc2)cc1 ; c1ccc(-c2ccc(-c3ccc4c(oc5ccc6oc7ccccc7c6c54)c3N(c3ccccc3)c3ccc4c(c3)C3(c5ccccc5-c5ccccc53)c3ccccc3-4)cc2)cc1]

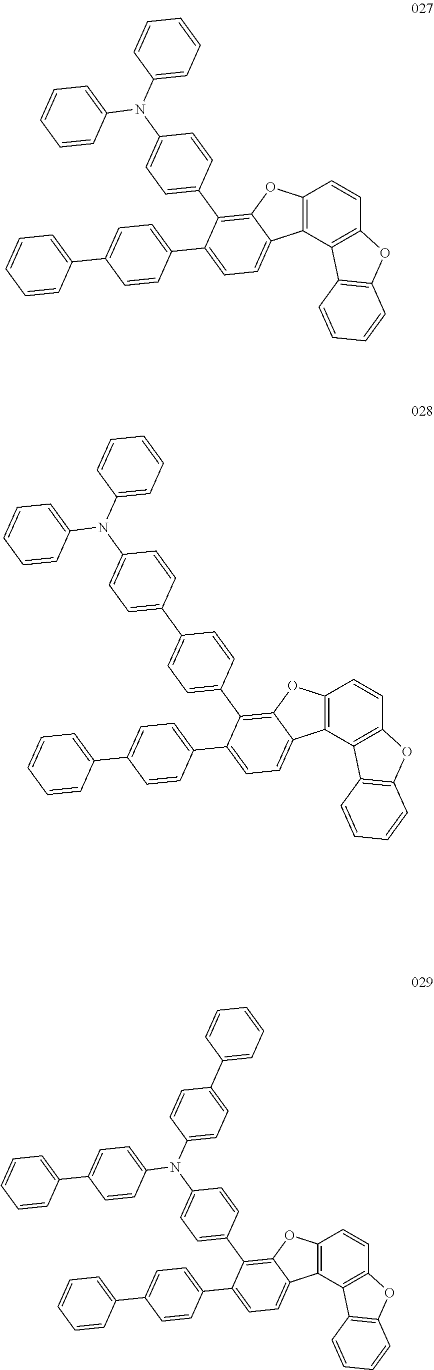 [SMILES: c1ccc(-c2ccc(-c3ccc4c(oc5ccc6oc7ccccc7c6c54)c3-c3ccc(N(c4ccccc4)c4ccccc4)cc3)cc2)cc1 ; c1ccc(-c2ccc(-c3ccc4c(oc5ccc6oc7ccccc7c6c54)c3-c3ccc(-c4ccc(N(c5ccccc5)c5ccccc5)cc4)cc3)cc2)cc1 ; c1ccc(-c2ccc(-c3ccc4c(oc5ccc6oc7ccccc7c6c54)c3-c3ccc(N(c4ccc(-c5ccccc5)cc4)c4ccc(-c5ccccc5)cc4)cc3)cc2)cc1]

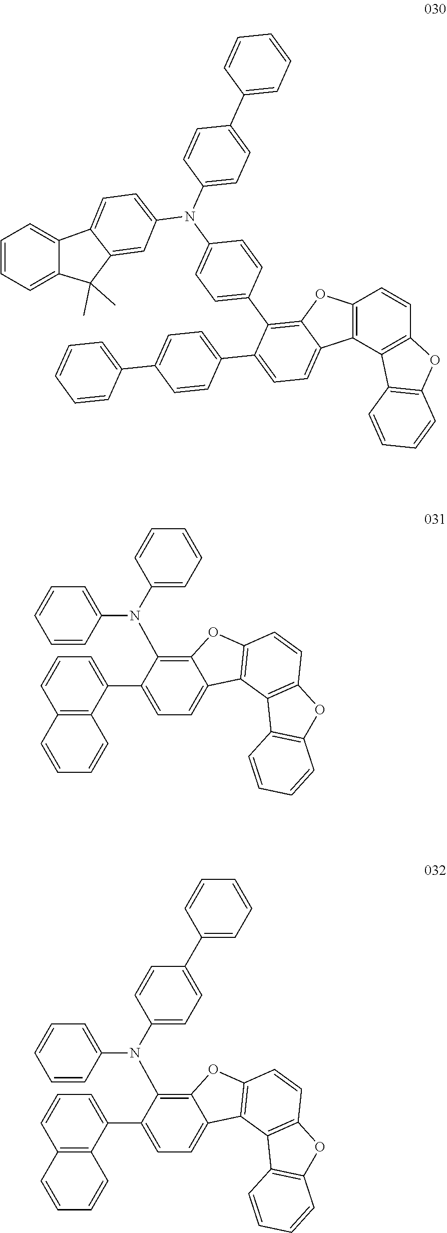 [SMILES: CC1(C)c2ccccc2-c2ccc(N(c3ccc(-c4ccccc4)cc3)c3ccc(-c4c(-c5ccc(-c6ccccc6)cc5)ccc5c4oc4ccc6oc7ccccc7c6c45)cc3)cc21 ; c1ccc(N(c2ccccc2)c2c(-c3cccc4ccccc34)ccc3c2oc2ccc4oc5ccccc5c4c23)cc1 ; c1ccc(-c2ccc(N(c3ccccc3)c3c(-c4cccc5ccccc45)ccc4c3oc3ccc5oc6ccccc6c5c34)cc2)cc1]

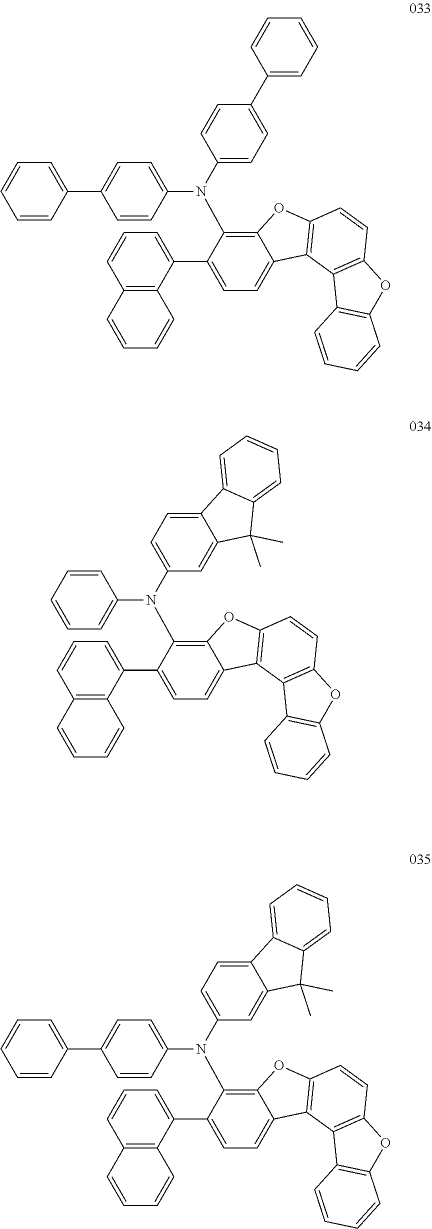 [SMILES: c1ccc(-c2ccc(N(c3ccc(-c4ccccc4)cc3)c3c(-c4cccc5ccccc45)ccc4c3oc3ccc5oc6ccccc6c5c34)cc2)cc1 ; CC1(C)c2ccccc2-c2ccc(N(c3ccccc3)c3c(-c4cccc5ccccc45)ccc4c3oc3ccc5oc6ccccc6c5c34)cc21 ; CC1(C)c2ccccc2-c2ccc(N(c3ccc(-c4ccccc4)cc3)c3c(-c4cccc5ccccc45)ccc4c3oc3ccc5oc6ccccc6c5c34)cc21]

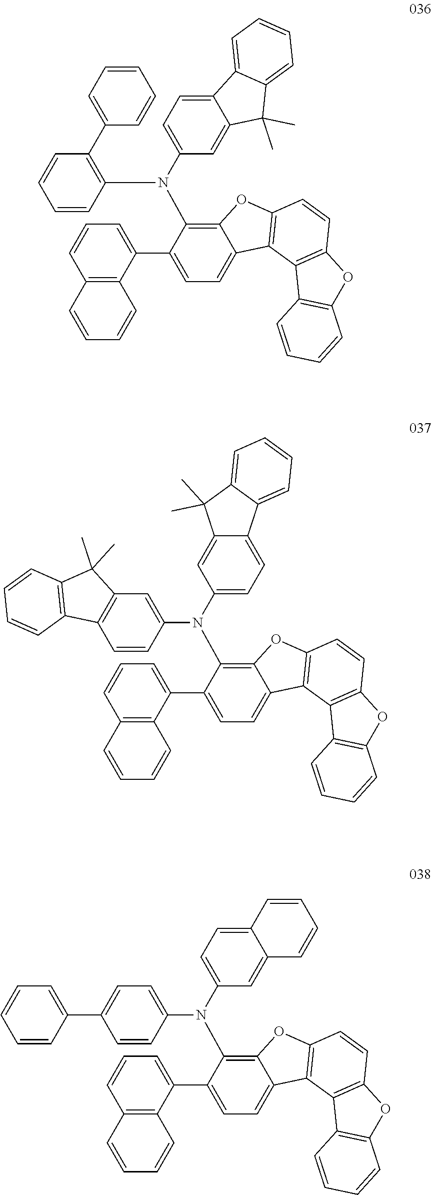 [SMILES: CC1(C)c2ccccc2-c2ccc(N(c3ccccc3-c3ccccc3)c3c(-c4cccc5ccccc45)ccc4c3oc3ccc5oc6ccccc6c5c34)cc21 ; CC1(C)c2ccccc2-c2ccc(N(c3ccc4c(c3)C(C)(C)c3ccccc3-4)c3c(-c4cccc5ccccc45)ccc4c3oc3ccc5oc6ccccc6c5c34)cc21 ; c1ccc(-c2ccc(N(c3ccc4ccccc4c3)c3c(-c4cccc5ccccc45)ccc4c3oc3ccc5oc6ccccc6c5c34)cc2)cc1]

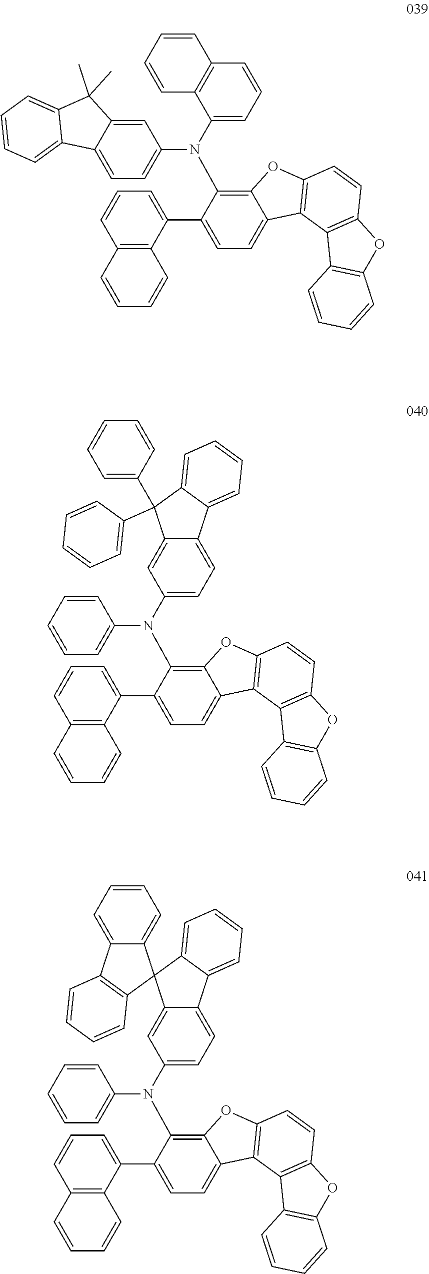 [SMILES: CC1(C)c2ccccc2-c2ccc(N(c3cccc4ccccc34)c3c(-c4cccc5ccccc45)ccc4c3oc3ccc5oc6ccccc6c5c34)cc21 ; c1ccc(N(c2ccc3c(c2)C(c2ccccc2)(c2ccccc2)c2ccccc2-3)c2c(-c3cccc4ccccc34)ccc3c2oc2ccc4oc5ccccc5c4c23)cc1 ; c1ccc(N(c2ccc3c(c2)C2(c4ccccc4-c4ccccc42)c2ccccc2-3)c2c(-c3cccc4ccccc34)ccc3c2oc2ccc4oc5ccccc5c4c23)cc1]

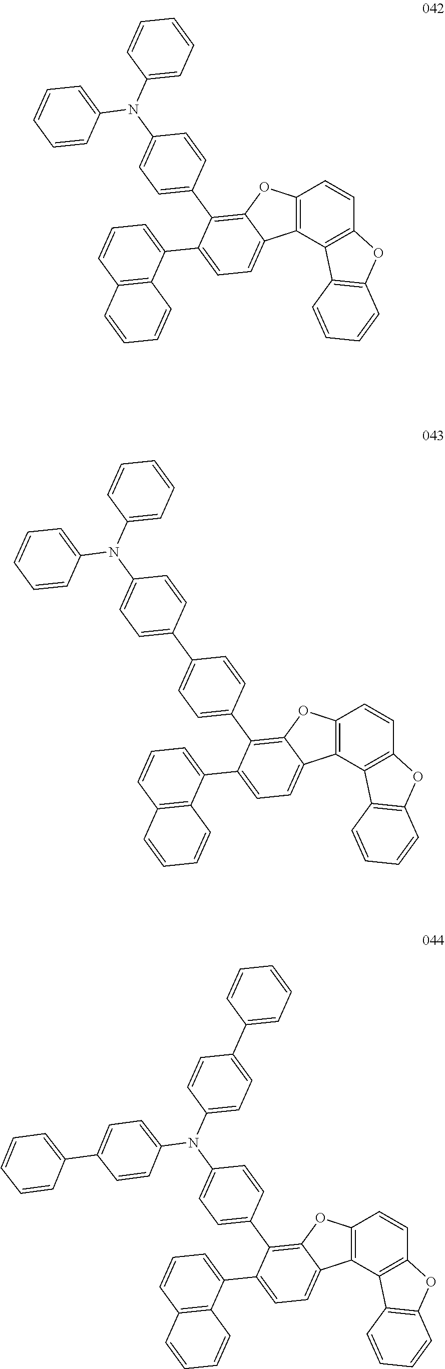 [SMILES: c1ccc(N(c2ccccc2)c2ccc(-c3c(-c4cccc5ccccc45)ccc4c3oc3ccc5oc6ccccc6c5c34)cc2)cc1 ; c1ccc(N(c2ccccc2)c2ccc(-c3ccc(-c4c(-c5cccc6ccccc56)ccc5c4oc4ccc6oc7ccccc7c6c45)cc3)cc2)cc1 ; c1ccc(-c2ccc(N(c3ccc(-c4ccccc4)cc3)c3ccc(-c4c(-c5cccc6ccccc56)ccc5c4oc4ccc6oc7ccccc7c6c45)cc3)cc2)cc1]

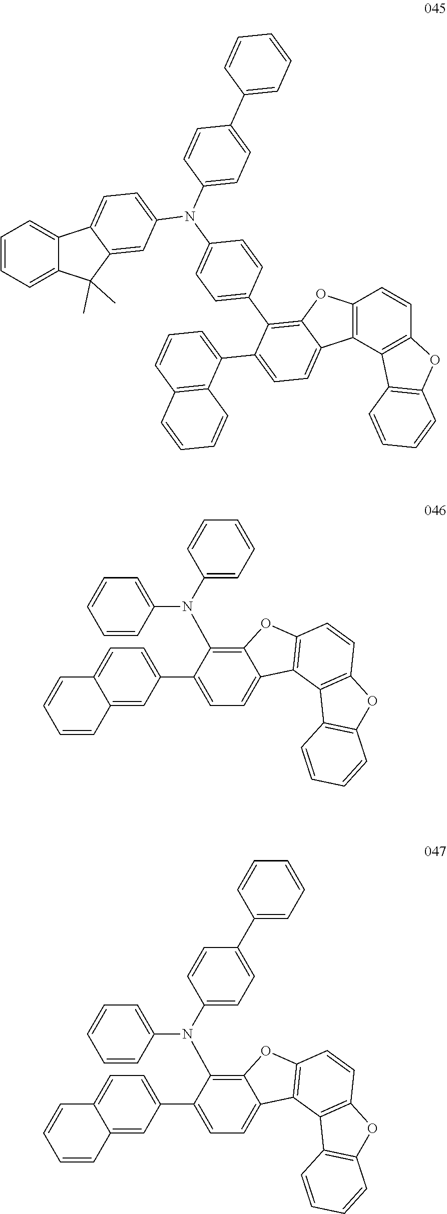 [SMILES: CC1(C)c2ccccc2-c2ccc(N(c3ccc(-c4ccccc4)cc3)c3ccc(-c4c(-c5cccc6ccccc56)ccc5c4oc4ccc6oc7ccccc7c6c45)cc3)cc21 ; c1ccc(N(c2ccccc2)c2c(-c3ccc4ccccc4c3)ccc3c2oc2ccc4oc5ccccc5c4c23)cc1 ; c1ccc(-c2ccc(N(c3ccccc3)c3c(-c4ccc5ccccc5c4)ccc4c3oc3ccc5oc6ccccc6c5c34)cc2)cc1]

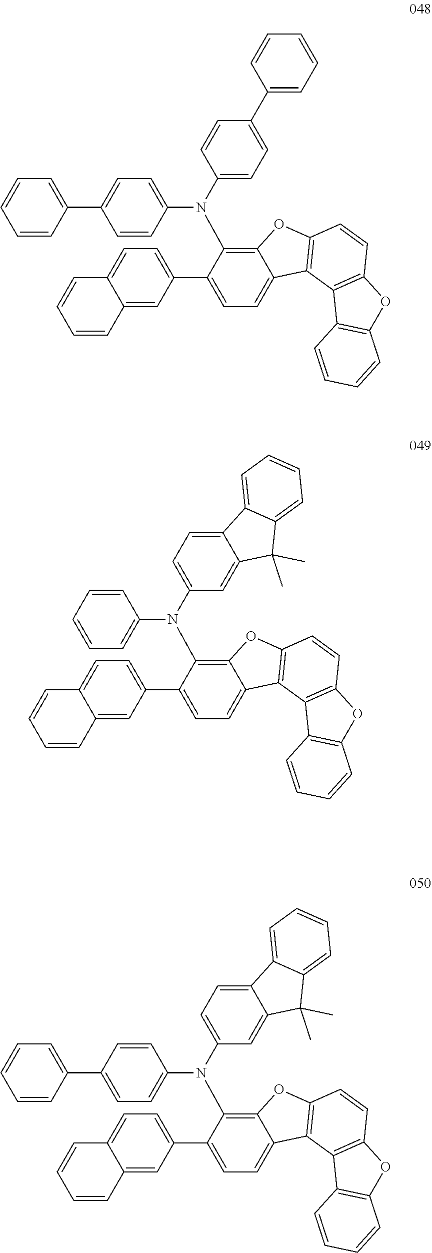 [SMILES: c1ccc(-c2ccc(N(c3ccc(-c4ccccc4)cc3)c3c(-c4ccc5ccccc5c4)ccc4c3oc3ccc5oc6ccccc6c5c34)cc2)cc1 ; CC1(C)c2ccccc2-c2ccc(N(c3ccccc3)c3c(-c4ccc5ccccc5c4)ccc4c3oc3ccc5oc6ccccc6c5c34)cc21 ; CC1(C)c2ccccc2-c2ccc(N(c3ccc(-c4ccccc4)cc3)c3c(-c4ccc5ccccc5c4)ccc4c3oc3ccc5oc6ccccc6c5c34)cc21]

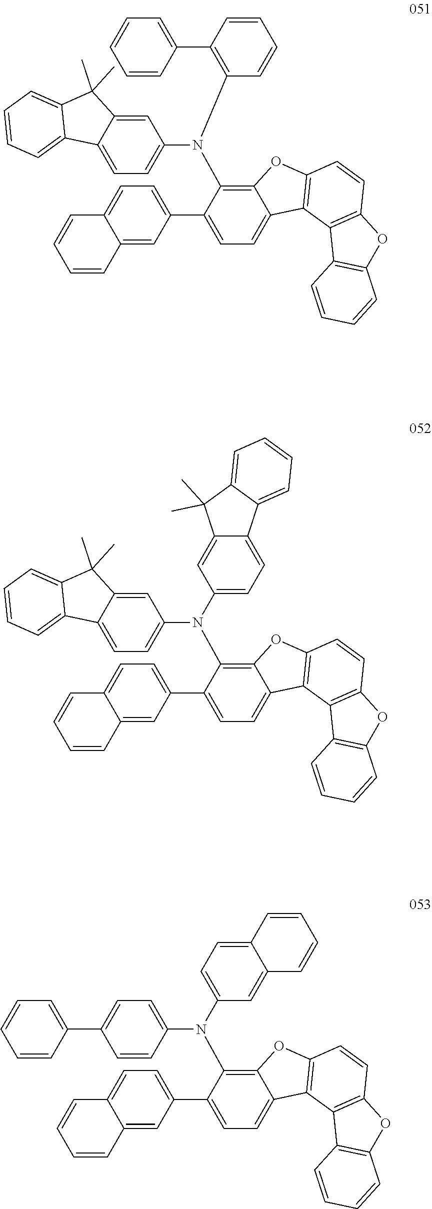 [SMILES: CC1(C)c2ccccc2-c2ccc(N(c3ccccc3-c3ccccc3)c3c(-c4ccc5ccccc5c4)ccc4c3oc3ccc5oc6ccccc6c5c34)cc21 ; CC1(C)c2ccccc2-c2ccc(N(c3ccc4c(c3)C(C)(C)c3ccccc3-4)c3c(-c4ccc5ccccc5c4)ccc4c3oc3ccc5oc6ccccc6c5c34)cc21 ; c1ccc(-c2ccc(N(c3ccc4ccccc4c3)c3c(-c4ccc5ccccc5c4)ccc4c3oc3ccc5oc6ccccc6c5c34)cc2)cc1]

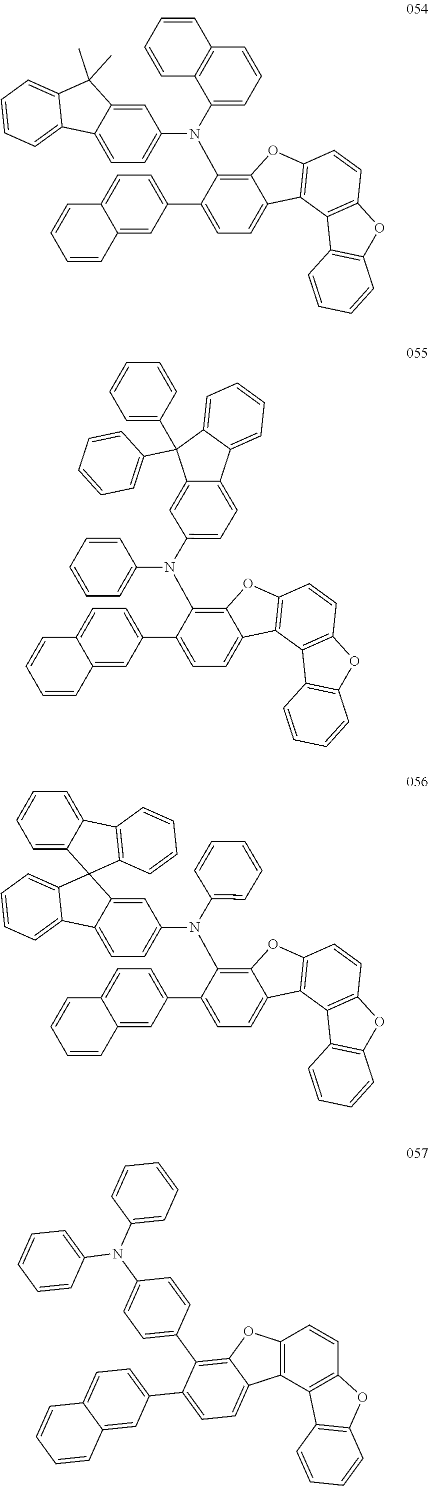 [SMILES: CC1(C)c2ccccc2-c2ccc(N(c3cccc4ccccc34)c3c(-c4ccc5ccccc5c4)ccc4c3oc3ccc5oc6ccccc6c5c34)cc21 ; c1ccc(N(c2ccc3c(c2)C(c2ccccc2)(c2ccccc2)c2ccccc2-3)c2c(-c3ccc4ccccc4c3)ccc3c2oc2ccc4oc5ccccc5c4c23)cc1 ; c1ccc(N(c2ccc3c(c2)C2(c4ccccc4-c4ccccc42)c2ccccc2-3)c2c(-c3ccc4ccccc4c3)ccc3c2oc2ccc4oc5ccccc5c4c23)cc1 ; c1ccc(N(c2ccccc2)c2ccc(-c3c(-c4ccc5ccccc5c4)ccc4c3oc3ccc5oc6ccccc6c5c34)cc2)cc1]

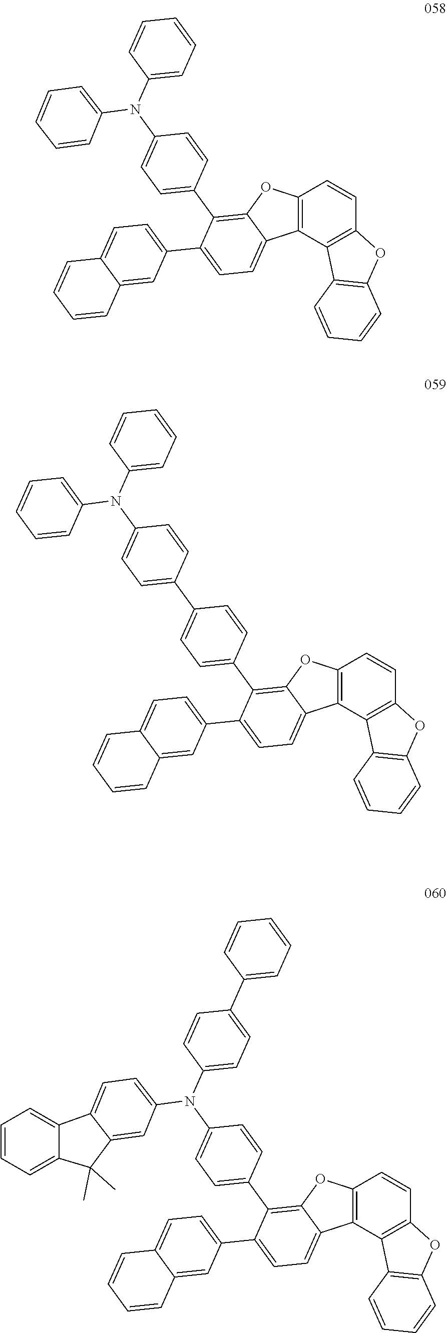 [SMILES: c1ccc(N(c2ccccc2)c2ccc(-c3c(-c4ccc5ccccc5c4)ccc4c3oc3ccc5oc6ccccc6c5c34)cc2)cc1 ; c1ccc(N(c2ccccc2)c2ccc(-c3ccc(-c4c(-c5ccc6ccccc6c5)ccc5c4oc4ccc6oc7ccccc7c6c45)cc3)cc2)cc1 ; CC1(C)c2ccccc2-c2ccc(N(c3ccc(-c4ccccc4)cc3)c3ccc(-c4c(-c5ccc6ccccc6c5)ccc5c4oc4ccc6oc7ccccc7c6c45)cc3)cc21]

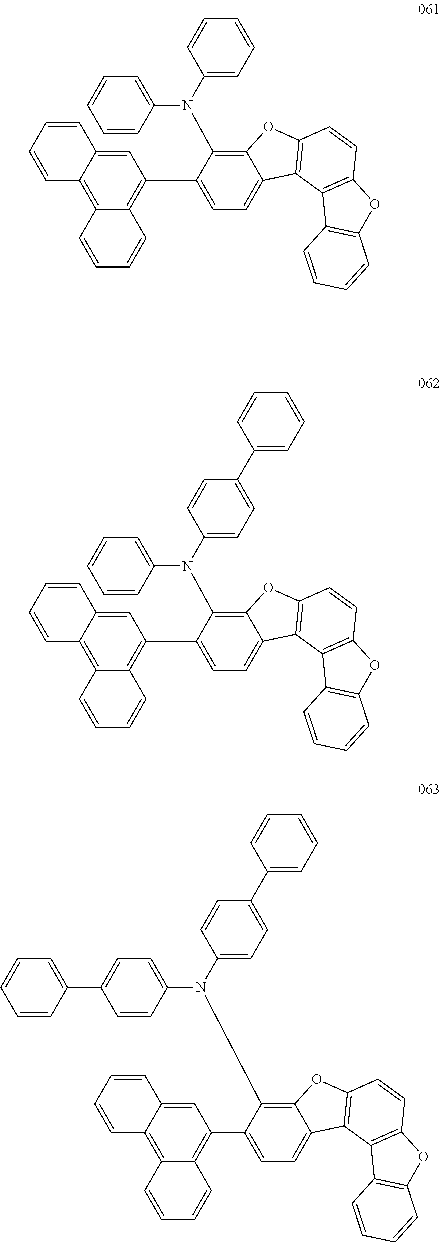 [SMILES: c1ccc(N(c2ccccc2)c2c(-c3cc4ccccc4c4ccccc34)ccc3c2oc2ccc4oc5ccccc5c4c23)cc1 ; c1ccc(-c2ccc(N(c3ccccc3)c3c(-c4cc5ccccc5c5ccccc45)ccc4c3oc3ccc5oc6ccccc6c5c34)cc2)cc1 ; c1ccc(-c2ccc(N(c3ccc(-c4ccccc4)cc3)c3c(-c4cc5ccccc5c5ccccc45)ccc4c3oc3ccc5oc6ccccc6c5c34)cc2)cc1]

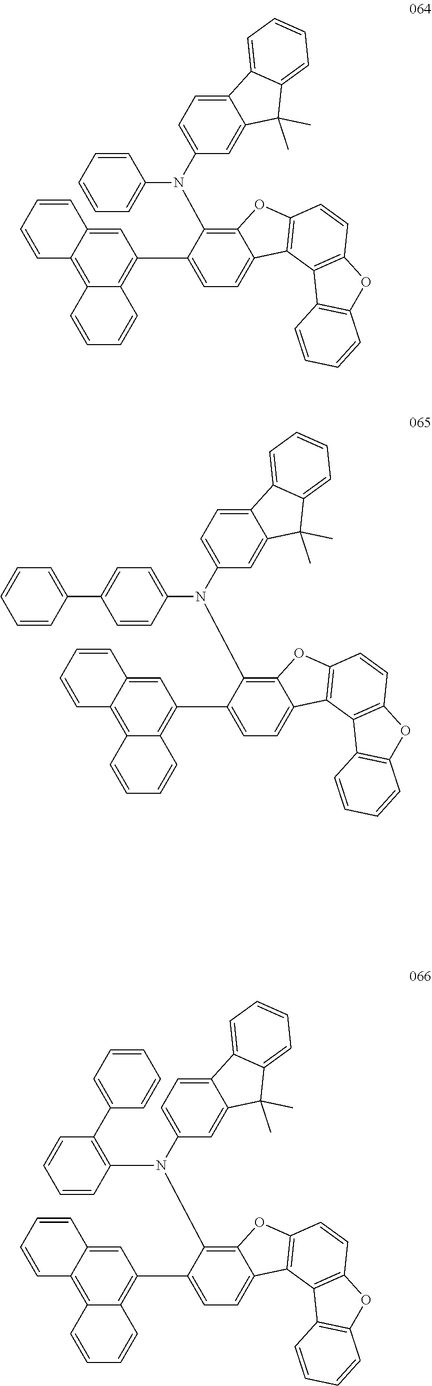 [SMILES: CC1(C)c2ccccc2-c2ccc(N(c3ccccc3)c3c(-c4cc5ccccc5c5ccccc45)ccc4c3oc3ccc5oc6ccccc6c5c34)cc21 ; CC1(C)c2ccccc2-c2ccc(N(c3ccc(-c4ccccc4)cc3)c3c(-c4cc5ccccc5c5ccccc45)ccc4c3oc3ccc5oc6ccccc6c5c34)cc21 ; CC1(C)c2ccccc2-c2ccc(N(c3ccccc3-c3ccccc3)c3c(-c4cc5ccccc5c5ccccc45)ccc4c3oc3ccc5oc6ccccc6c5c34)cc21]

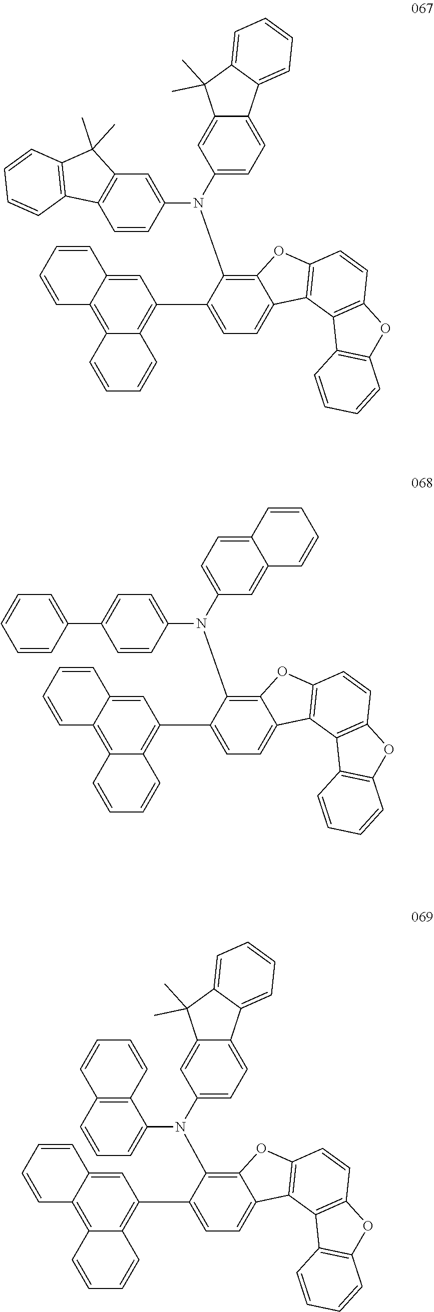 [SMILES: CC1(C)c2ccccc2-c2ccc(N(c3ccc4c(c3)C(C)(C)c3ccccc3-4)c3c(-c4cc5ccccc5c5ccccc45)ccc4c3oc3ccc5oc6ccccc6c5c34)cc21 ; c1ccc(-c2ccc(N(c3ccc4ccccc4c3)c3c(-c4cc5ccccc5c5ccccc45)ccc4c3oc3ccc5oc6ccccc6c5c34)cc2)cc1 ; CC1(C)c2ccccc2-c2ccc(N(c3cccc4ccccc34)c3c(-c4cc5ccccc5c5ccccc45)ccc4c3oc3ccc5oc6ccccc6c5c34)cc21]

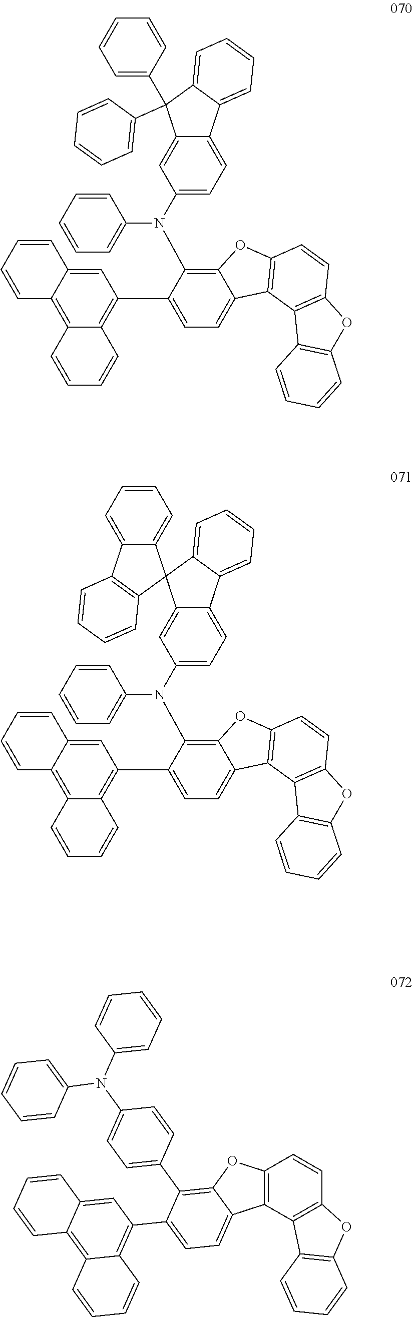 [SMILES: c1ccc(N(c2ccc3c(c2)C(c2ccccc2)(c2ccccc2)c2ccccc2-3)c2c(-c3cc4ccccc4c4ccccc34)ccc3c2oc2ccc4oc5ccccc5c4c23)cc1 ; c1ccc(N(c2ccc3c(c2)C2(c4ccccc4-c4ccccc42)c2ccccc2-3)c2c(-c3cc4ccccc4c4ccccc34)ccc3c2oc2ccc4oc5ccccc5c4c23)cc1 ; c1ccc(N(c2ccccc2)c2ccc(-c3c(-c4cc5ccccc5c5ccccc45)ccc4c3oc3ccc5oc6ccccc6c5c34)cc2)cc1]

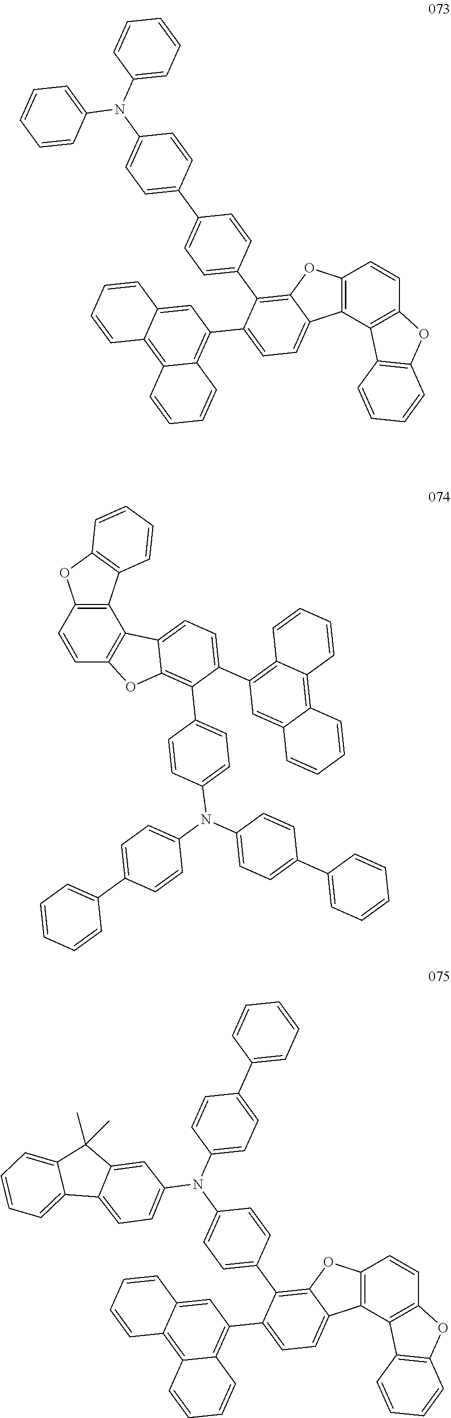 [SMILES: c1ccc(N(c2ccccc2)c2ccc(-c3ccc(-c4c(-c5cc6ccccc6c6ccccc56)ccc5c4oc4ccc6oc7ccccc7c6c45)cc3)cc2)cc1 ; c1ccc(-c2ccc(N(c3ccc(-c4ccccc4)cc3)c3ccc(-c4c(-c5cc6ccccc6c6ccccc56)ccc5c4oc4ccc6oc7ccccc7c6c45)cc3)cc2)cc1 ; CC1(C)c2ccccc2-c2ccc(N(c3ccc(-c4ccccc4)cc3)c3ccc(-c4c(-c5cc6ccccc6c6ccccc56)ccc5c4oc4ccc6oc7ccccc7c6c45)cc3)cc21]

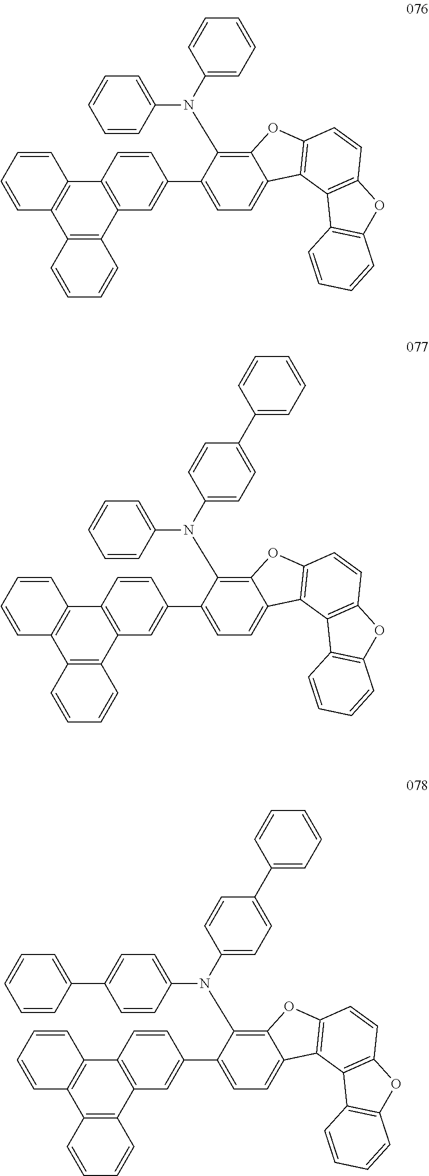 [SMILES: c1ccc(N(c2ccccc2)c2c(-c3ccc4c5ccccc5c5ccccc5c4c3)ccc3c2oc2ccc4oc5ccccc5c4c23)cc1 ; c1ccc(-c2ccc(N(c3ccccc3)c3c(-c4ccc5c6ccccc6c6ccccc6c5c4)ccc4c3oc3ccc5oc6ccccc6c5c34)cc2)cc1 ; c1ccc(-c2ccc(N(c3ccc(-c4ccccc4)cc3)c3c(-c4ccc5c6ccccc6c6ccccc6c5c4)ccc4c3oc3ccc5oc6ccccc6c5c34)cc2)cc1]

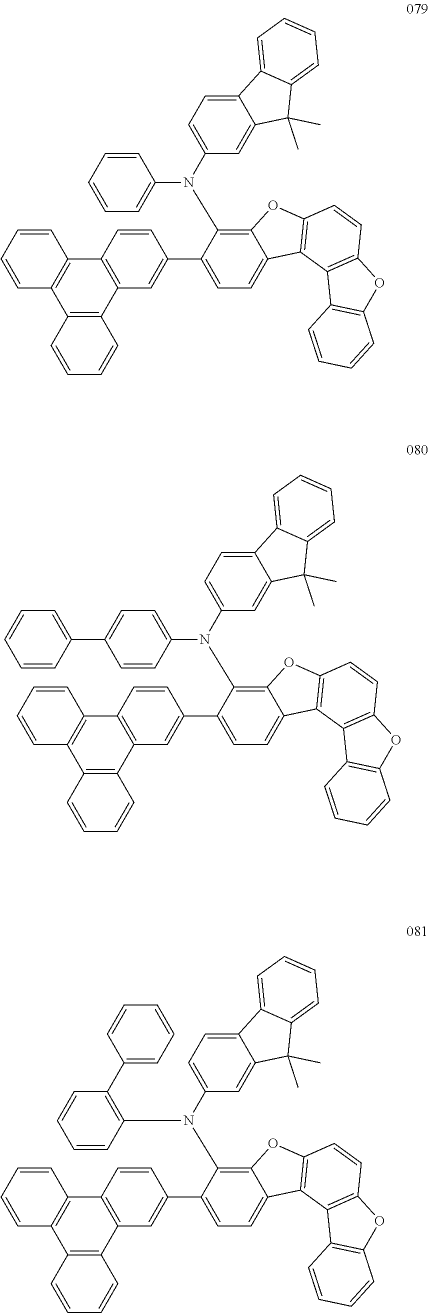 [SMILES: CC1(C)c2ccccc2-c2ccc(N(c3ccccc3)c3c(-c4ccc5c6ccccc6c6ccccc6c5c4)ccc4c3oc3ccc5oc6ccccc6c5c34)cc21 ; CC1(C)c2ccccc2-c2ccc(N(c3ccc(-c4ccccc4)cc3)c3c(-c4ccc5c6ccccc6c6ccccc6c5c4)ccc4c3oc3ccc5oc6ccccc6c5c34)cc21 ; CC1(C)c2ccccc2-c2ccc(N(c3ccccc3-c3ccccc3)c3c(-c4ccc5c6ccccc6c6ccccc6c5c4)ccc4c3oc3ccc5oc6ccccc6c5c34)cc21]

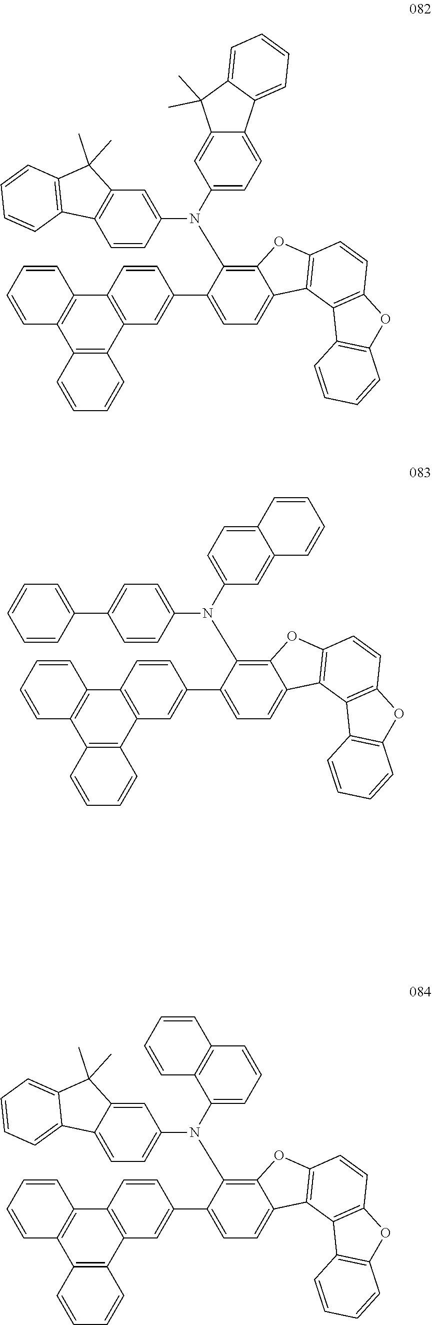 [SMILES: CC1(C)c2ccccc2-c2ccc(N(c3ccc4c(c3)C(C)(C)c3ccccc3-4)c3c(-c4ccc5c6ccccc6c6ccccc6c5c4)ccc4c3oc3ccc5oc6ccccc6c5c34)cc21 ; c1ccc(-c2ccc(N(c3ccc4ccccc4c3)c3c(-c4ccc5c6ccccc6c6ccccc6c5c4)ccc4c3oc3ccc5oc6ccccc6c5c34)cc2)cc1 ; CC1(C)c2ccccc2-c2ccc(N(c3cccc4ccccc34)c3c(-c4ccc5c6ccccc6c6ccccc6c5c4)ccc4c3oc3ccc5oc6ccccc6c5c34)cc21]

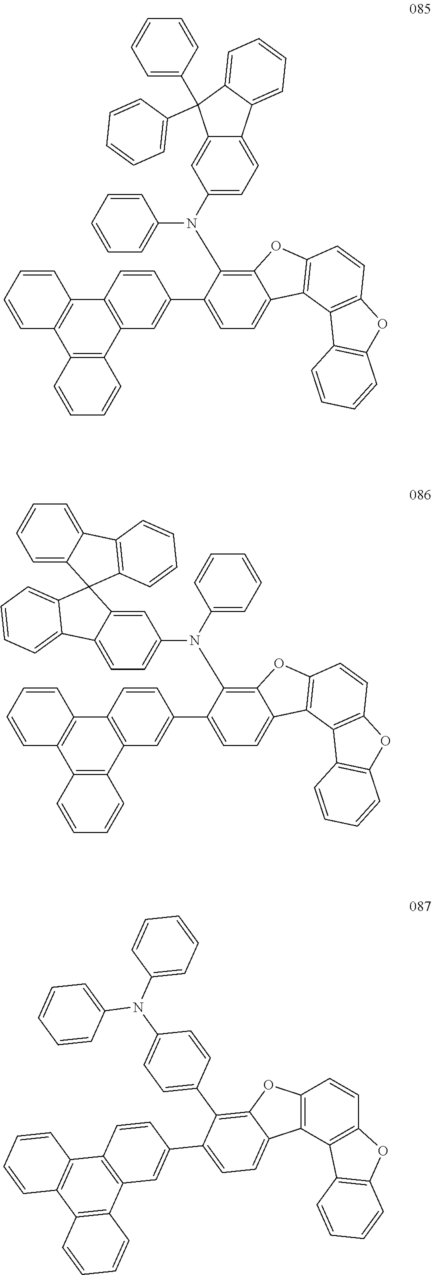 [SMILES: c1ccc(N(c2ccc3c(c2)C(c2ccccc2)(c2ccccc2)c2ccccc2-3)c2c(-c3ccc4c5ccccc5c5ccccc5c4c3)ccc3c2oc2ccc4oc5ccccc5c4c23)cc1 ; c1ccc(N(c2ccc3c(c2)C2(c4ccccc4-c4ccccc42)c2ccccc2-3)c2c(-c3ccc4c5ccccc5c5ccccc5c4c3)ccc3c2oc2ccc4oc5ccccc5c4c23)cc1 ; c1ccc(N(c2ccccc2)c2ccc(-c3c(-c4ccc5c6ccccc6c6ccccc6c5c4)ccc4c3oc3ccc5oc6ccccc6c5c34)cc2)cc1]

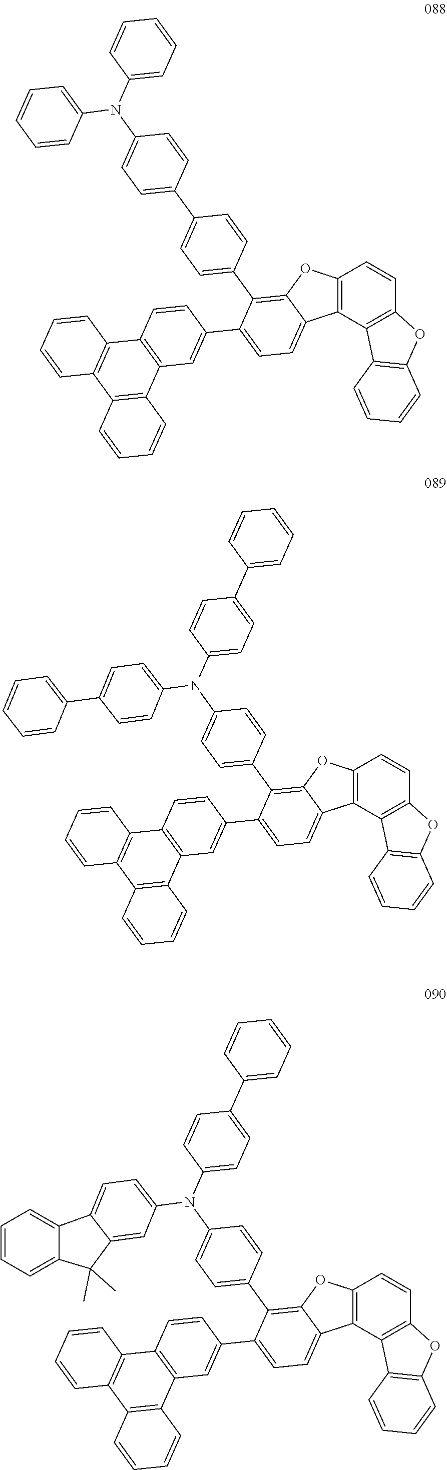 [SMILES: c1ccc(N(c2ccccc2)c2ccc(-c3ccc(-c4c(-c5ccc6c7ccccc7c7ccccc7c6c5)ccc5c4oc4ccc6oc7ccccc7c6c45)cc3)cc2)cc1 ; c1ccc(-c2ccc(N(c3ccc(-c4ccccc4)cc3)c3ccc(-c4c(-c5ccc6c7ccccc7c7ccccc7c6c5)ccc5c4oc4ccc6oc7ccccc7c6c45)cc3)cc2)cc1 ; CC1(C)c2ccccc2-c2ccc(N(c3ccc(-c4ccccc4)cc3)c3ccc(-c4c(-c5ccc6c7ccccc7c7ccccc7c6c5)ccc5c4oc4ccc6oc7ccccc7c6c45)cc3)cc21]

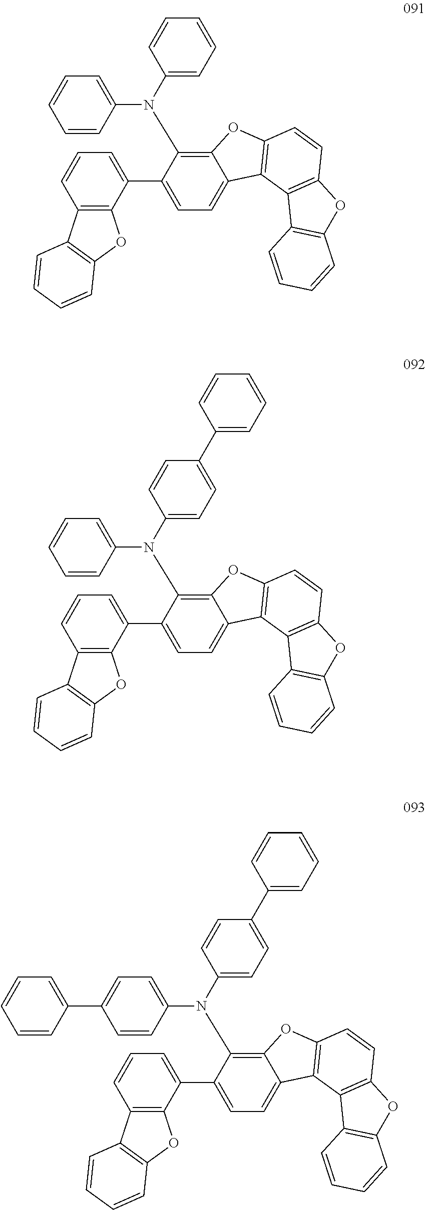 [SMILES: c1ccc(N(c2ccccc2)c2c(-c3cccc4c3oc3ccccc34)ccc3c2oc2ccc4oc5ccccc5c4c23)cc1 ; c1ccc(-c2ccc(N(c3ccccc3)c3c(-c4cccc5c4oc4ccccc45)ccc4c3oc3ccc5oc6ccccc6c5c34)cc2)cc1 ; c1ccc(-c2ccc(N(c3ccc(-c4ccccc4)cc3)c3c(-c4cccc5c4oc4ccccc45)ccc4c3oc3ccc5oc6ccccc6c5c34)cc2)cc1]

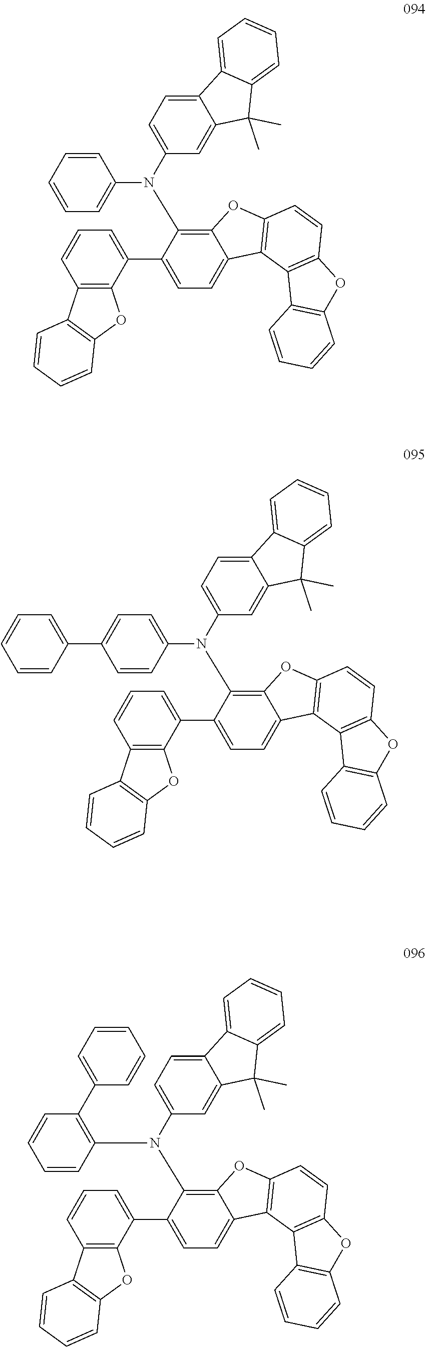 [SMILES: CC1(C)c2ccccc2-c2ccc(N(c3ccccc3)c3c(-c4cccc5c4oc4ccccc45)ccc4c3oc3ccc5oc6ccccc6c5c34)cc21 ; CC1(C)c2ccccc2-c2ccc(N(c3ccc(-c4ccccc4)cc3)c3c(-c4cccc5c4oc4ccccc45)ccc4c3oc3ccc5oc6ccccc6c5c34)cc21 ; CC1(C)c2ccccc2-c2ccc(N(c3ccccc3-c3ccccc3)c3c(-c4cccc5c4oc4ccccc45)ccc4c3oc3ccc5oc6ccccc6c5c34)cc21]

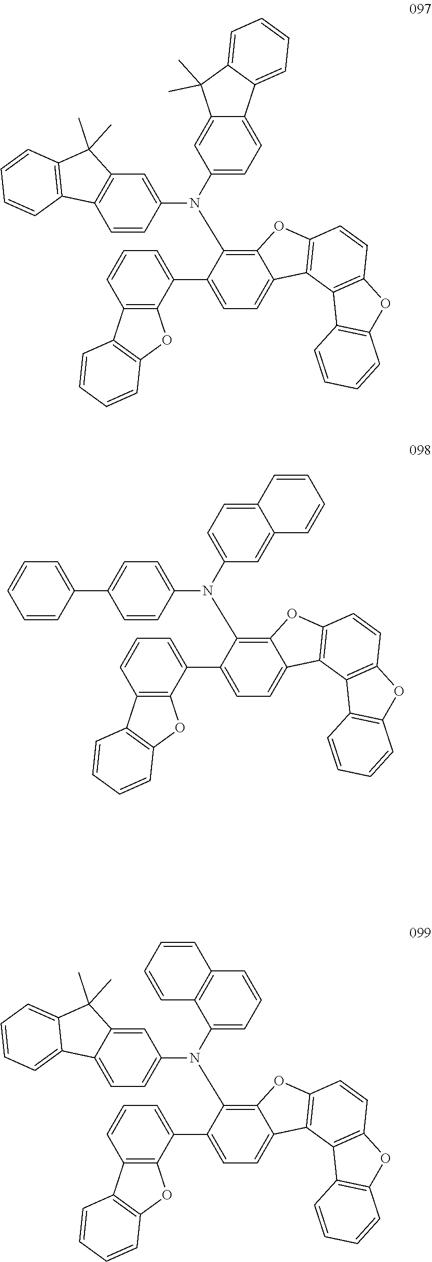 [SMILES: CC1(C)c2ccccc2-c2ccc(N(c3ccc4c(c3)C(C)(C)c3ccccc3-4)c3c(-c4cccc5c4oc4ccccc45)ccc4c3oc3ccc5oc6ccccc6c5c34)cc21 ; c1ccc(-c2ccc(N(c3ccc4ccccc4c3)c3c(-c4cccc5c4oc4ccccc45)ccc4c3oc3ccc5oc6ccccc6c5c34)cc2)cc1 ; CC1(C)c2ccccc2-c2ccc(N(c3cccc4ccccc34)c3c(-c4cccc5c4oc4ccccc45)ccc4c3oc3ccc5oc6ccccc6c5c34)cc21]

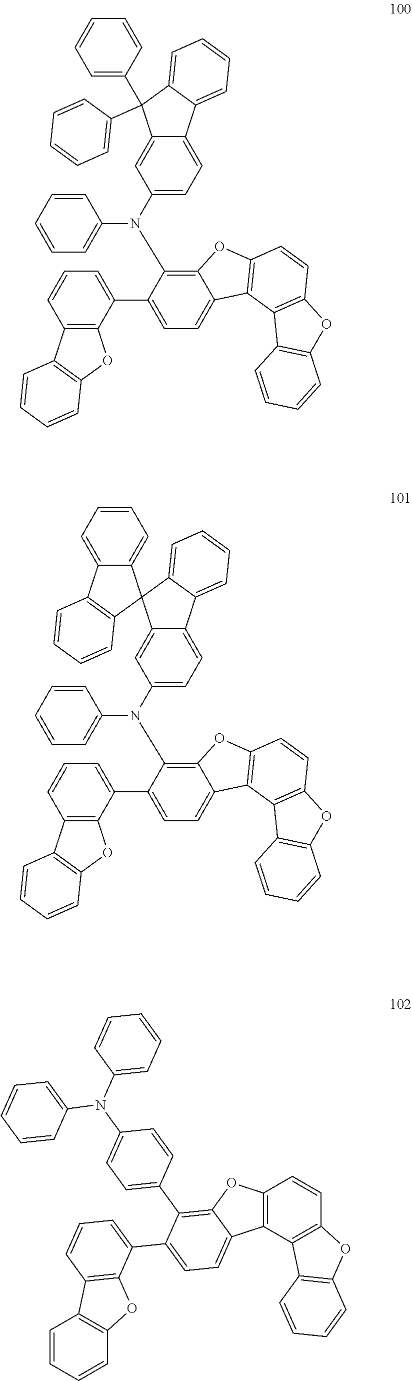 [SMILES: c1ccc(N(c2ccc3c(c2)C(c2ccccc2)(c2ccccc2)c2ccccc2-3)c2c(-c3cccc4c3oc3ccccc34)ccc3c2oc2ccc4oc5ccccc5c4c23)cc1 ; c1ccc(N(c2ccc3c(c2)C2(c4ccccc4-c4ccccc42)c2ccccc2-3)c2c(-c3cccc4c3oc3ccccc34)ccc3c2oc2ccc4oc5ccccc5c4c23)cc1 ; c1ccc(N(c2ccccc2)c2ccc(-c3c(-c4cccc5c4oc4ccccc45)ccc4c3oc3ccc5oc6ccccc6c5c34)cc2)cc1]

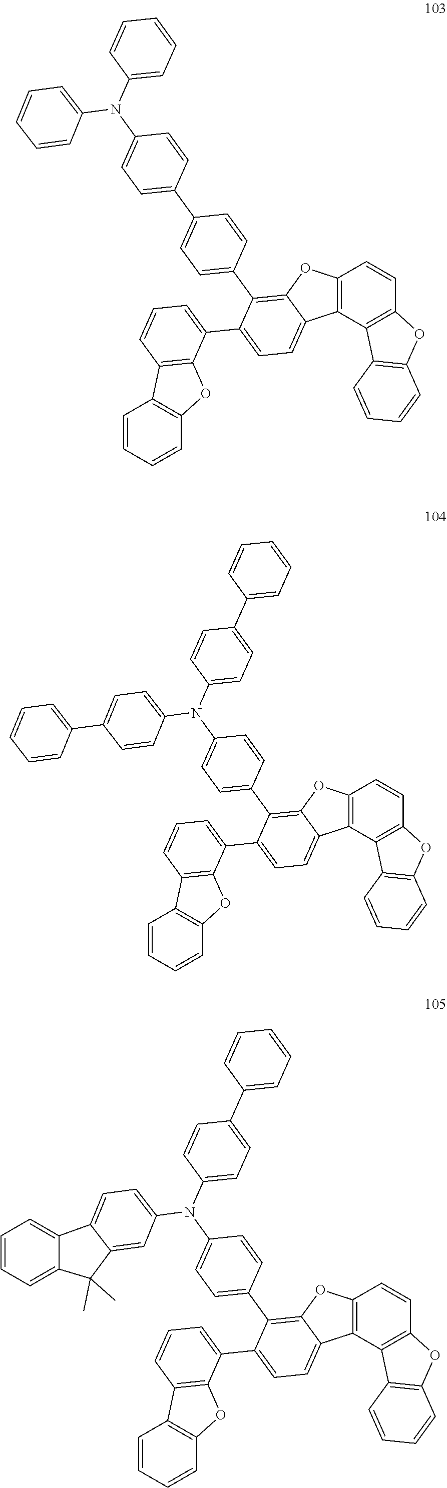 [SMILES: c1ccc(N(c2ccccc2)c2ccc(-c3ccc(-c4c(-c5cccc6c5oc5ccccc56)ccc5c4oc4ccc6oc7ccccc7c6c45)cc3)cc2)cc1 ; c1ccc(-c2ccc(N(c3ccc(-c4ccccc4)cc3)c3ccc(-c4c(-c5cccc6c5oc5ccccc56)ccc5c4oc4ccc6oc7ccccc7c6c45)cc3)cc2)cc1 ; CC1(C)c2ccccc2-c2ccc(N(c3ccc(-c4ccccc4)cc3)c3ccc(-c4c(-c5cccc6c5oc5ccccc56)ccc5c4oc4ccc6oc7ccccc7c6c45)cc3)cc21]

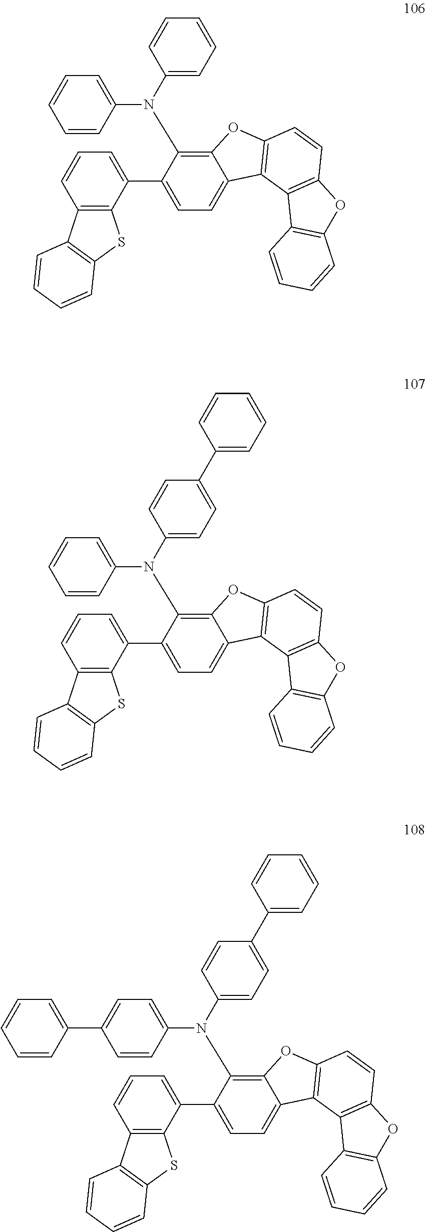 [SMILES: c1ccc(N(c2ccccc2)c2c(-c3cccc4c3sc3ccccc34)ccc3c2oc2ccc4oc5ccccc5c4c23)cc1 ; c1ccc(-c2ccc(N(c3ccccc3)c3c(-c4cccc5c4sc4ccccc45)ccc4c3oc3ccc5oc6ccccc6c5c34)cc2)cc1 ; c1ccc(-c2ccc(N(c3ccc(-c4ccccc4)cc3)c3c(-c4cccc5c4sc4ccccc45)ccc4c3oc3ccc5oc6ccccc6c5c34)cc2)cc1]

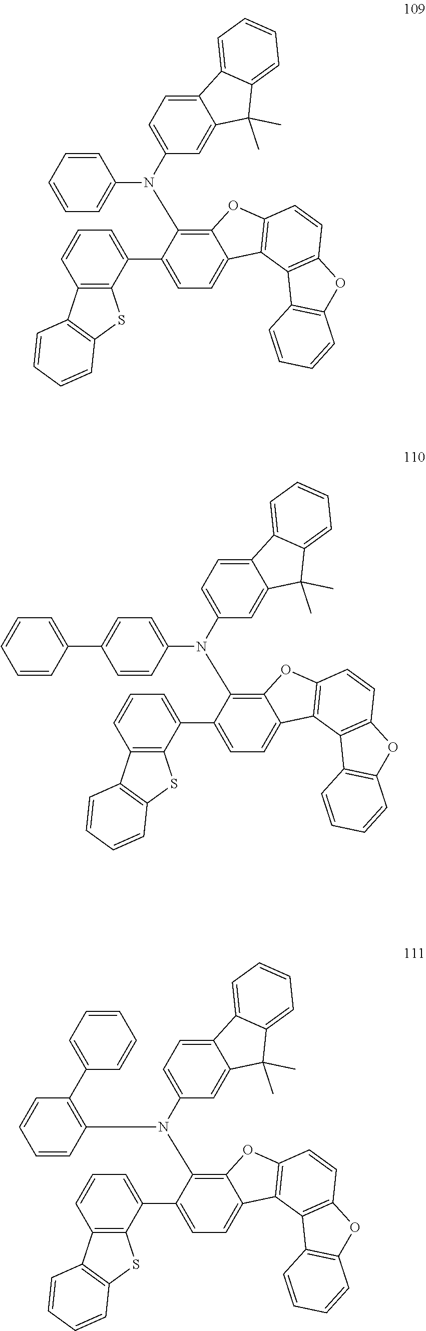 [SMILES: CC1(C)c2ccccc2-c2ccc(N(c3ccccc3)c3c(-c4cccc5c4sc4ccccc45)ccc4c3oc3ccc5oc6ccccc6c5c34)cc21 ; CC1(C)c2ccccc2-c2ccc(N(c3ccc(-c4ccccc4)cc3)c3c(-c4cccc5c4sc4ccccc45)ccc4c3oc3ccc5oc6ccccc6c5c34)cc21 ; CC1(C)c2ccccc2-c2ccc(N(c3ccccc3-c3ccccc3)c3c(-c4cccc5c4sc4ccccc45)ccc4c3oc3ccc5oc6ccccc6c5c34)cc21]

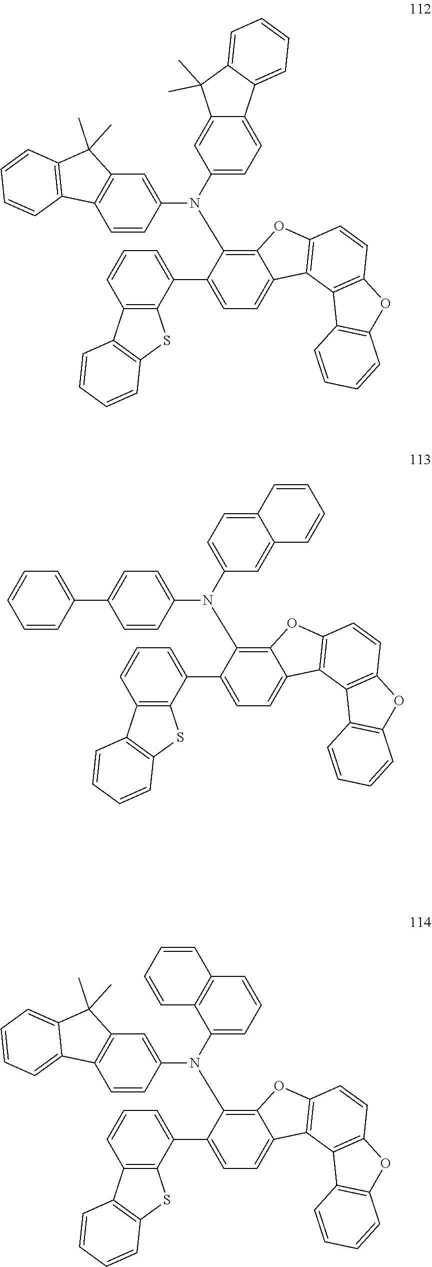 [SMILES: CC1(C)c2ccccc2-c2ccc(N(c3ccc4c(c3)C(C)(C)c3ccccc3-4)c3c(-c4cccc5c4sc4ccccc45)ccc4c3oc3ccc5oc6ccccc6c5c34)cc21 ; c1ccc(-c2ccc(N(c3ccc4ccccc4c3)c3c(-c4cccc5c4sc4ccccc45)ccc4c3oc3ccc5oc6ccccc6c5c34)cc2)cc1 ; CC1(C)c2ccccc2-c2ccc(N(c3cccc4ccccc34)c3c(-c4cccc5c4sc4ccccc45)ccc4c3oc3ccc5oc6ccccc6c5c34)cc21]

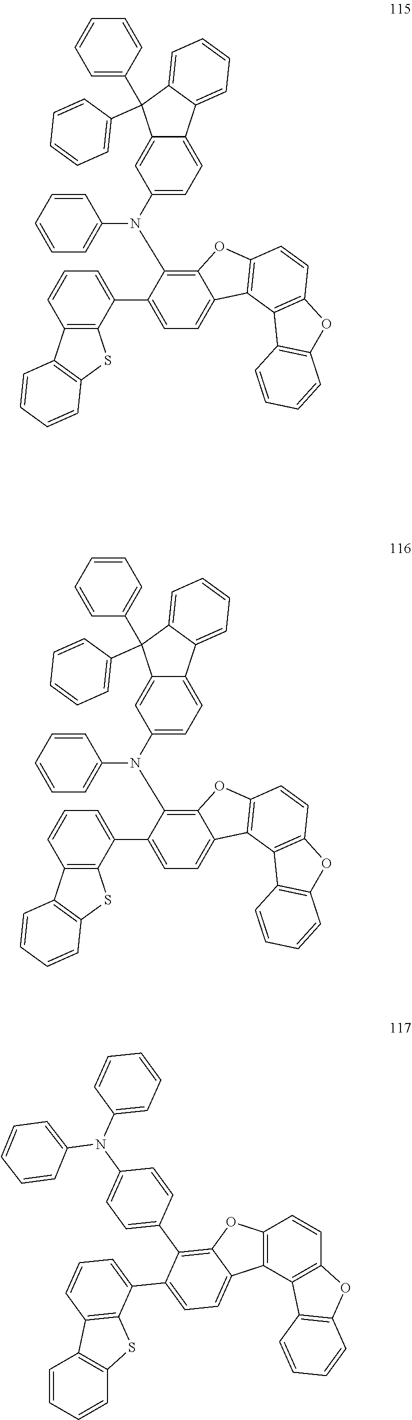 [SMILES: c1ccc(N(c2ccc3c(c2)C(c2ccccc2)(c2ccccc2)c2ccccc2-3)c2c(-c3cccc4c3sc3ccccc34)ccc3c2oc2ccc4oc5ccccc5c4c23)cc1 ; c1ccc(N(c2ccc3c(c2)C(c2ccccc2)(c2ccccc2)c2ccccc2-3)c2c(-c3cccc4c3sc3ccccc34)ccc3c2oc2ccc4oc5ccccc5c4c23)cc1 ; c1ccc(N(c2ccccc2)c2ccc(-c3c(-c4cccc5c4sc4ccccc45)ccc4c3oc3ccc5oc6ccccc6c5c34)cc2)cc1]

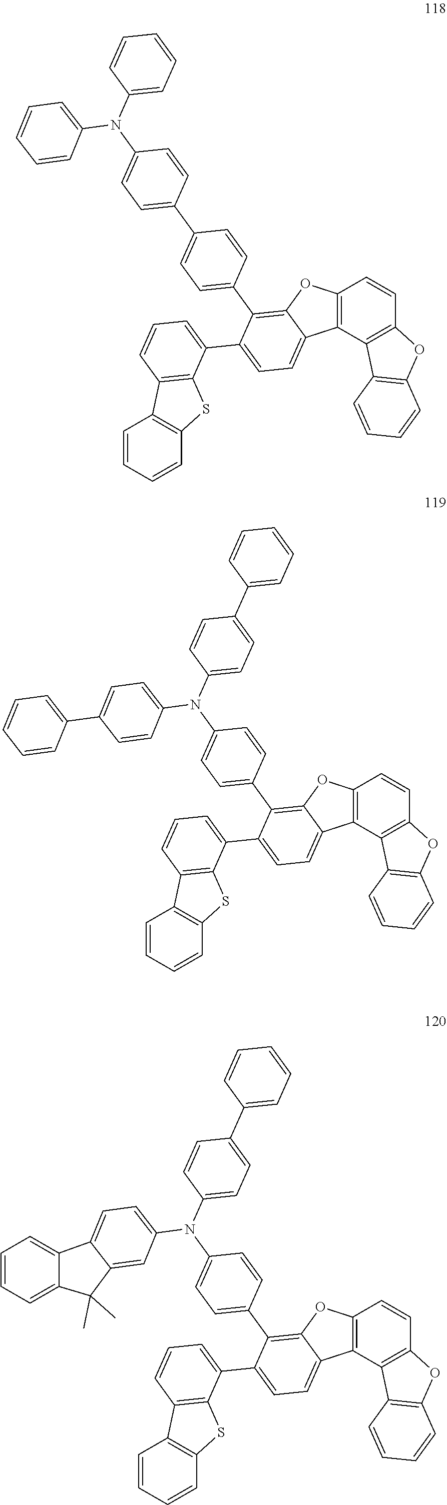 [SMILES: c1ccc(N(c2ccccc2)c2ccc(-c3ccc(-c4c(-c5cccc6c5sc5ccccc56)ccc5c4oc4ccc6oc7ccccc7c6c45)cc3)cc2)cc1 ; c1ccc(-c2ccc(N(c3ccc(-c4ccccc4)cc3)c3ccc(-c4c(-c5cccc6c5sc5ccccc56)ccc5c4oc4ccc6oc7ccccc7c6c45)cc3)cc2)cc1 ; CC1(C)c2ccccc2-c2ccc(N(c3ccc(-c4ccccc4)cc3)c3ccc(-c4c(-c5cccc6c5sc5ccccc56)ccc5c4oc4ccc6oc7ccccc7c6c45)cc3)cc21]

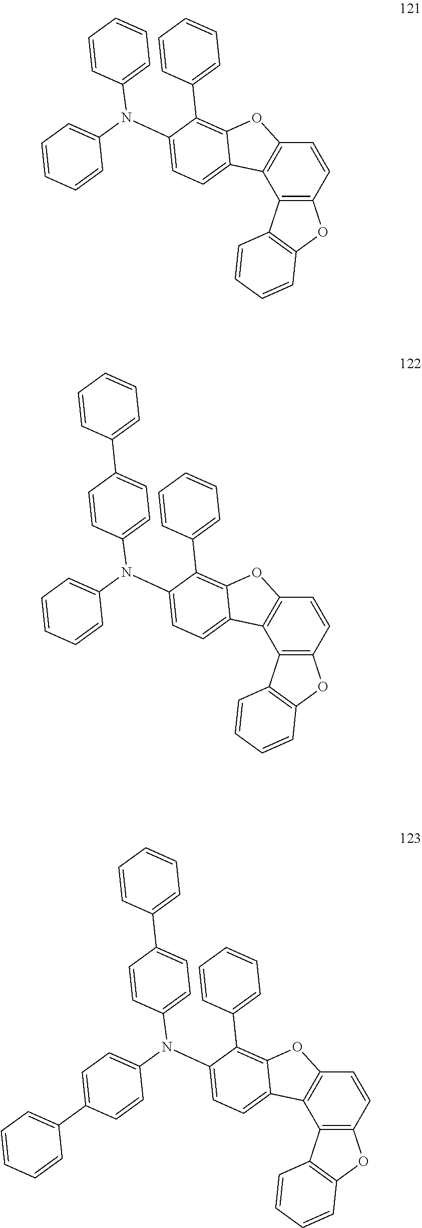 [SMILES: c1ccc(-c2c(N(c3ccccc3)c3ccccc3)ccc3c2oc2ccc4oc5ccccc5c4c23)cc1 ; c1ccc(-c2ccc(N(c3ccccc3)c3ccc4c(oc5ccc6oc7ccccc7c6c54)c3-c3ccccc3)cc2)cc1 ; c1ccc(-c2ccc(N(c3ccc(-c4ccccc4)cc3)c3ccc4c(oc5ccc6oc7ccccc7c6c54)c3-c3ccccc3)cc2)cc1]

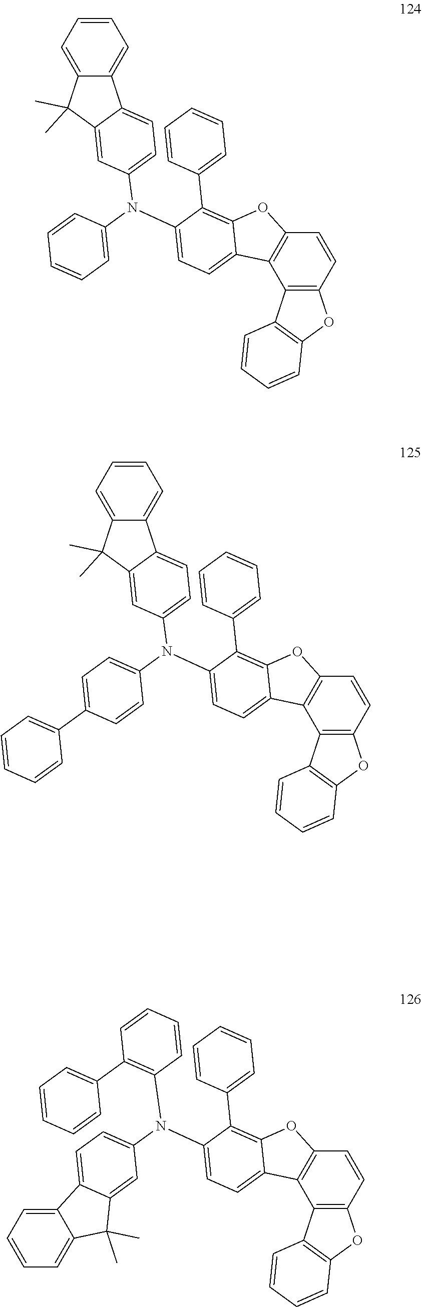 [SMILES: CC1(C)c2ccccc2-c2ccc(N(c3ccccc3)c3ccc4c(oc5ccc6oc7ccccc7c6c54)c3-c3ccccc3)cc21 ; CC1(C)c2ccccc2-c2ccc(N(c3ccc(-c4ccccc4)cc3)c3ccc4c(oc5ccc6oc7ccccc7c6c54)c3-c3ccccc3)cc21 ; CC1(C)c2ccccc2-c2ccc(N(c3ccccc3-c3ccccc3)c3ccc4c(oc5ccc6oc7ccccc7c6c54)c3-c3ccccc3)cc21]

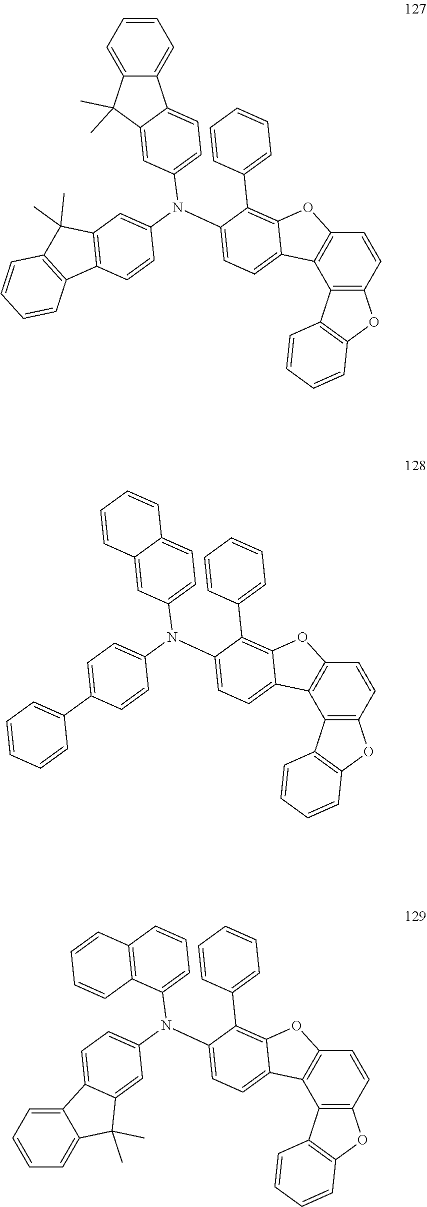 [SMILES: CC1(C)c2ccccc2-c2ccc(N(c3ccc4c(c3)C(C)(C)c3ccccc3-4)c3ccc4c(oc5ccc6oc7ccccc7c6c54)c3-c3ccccc3)cc21 ; c1ccc(-c2ccc(N(c3ccc4ccccc4c3)c3ccc4c(oc5ccc6oc7ccccc7c6c54)c3-c3ccccc3)cc2)cc1 ; CC1(C)c2ccccc2-c2ccc(N(c3ccc4c(oc5ccc6oc7ccccc7c6c54)c3-c3ccccc3)c3cccc4ccccc34)cc21]

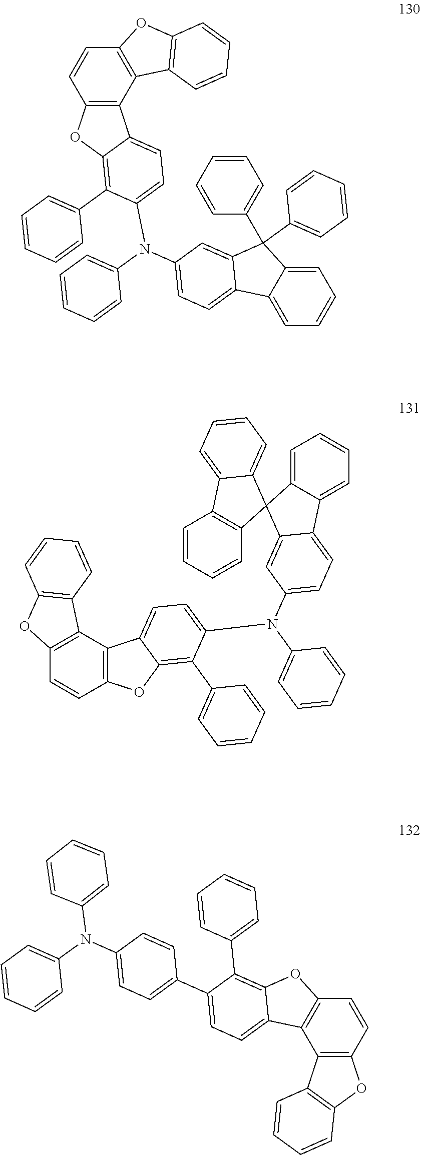 [SMILES: c1ccc(-c2c(N(c3ccccc3)c3ccc4c(c3)C(c3ccccc3)(c3ccccc3)c3ccccc3-4)ccc3c2oc2ccc4oc5ccccc5c4c23)cc1 ; c1ccc(-c2c(N(c3ccccc3)c3ccc4c(c3)C3(c5ccccc5-c5ccccc53)c3ccccc3-4)ccc3c2oc2ccc4oc5ccccc5c4c23)cc1 ; c1ccc(-c2c(-c3ccc(N(c4ccccc4)c4ccccc4)cc3)ccc3c2oc2ccc4oc5ccccc5c4c23)cc1]

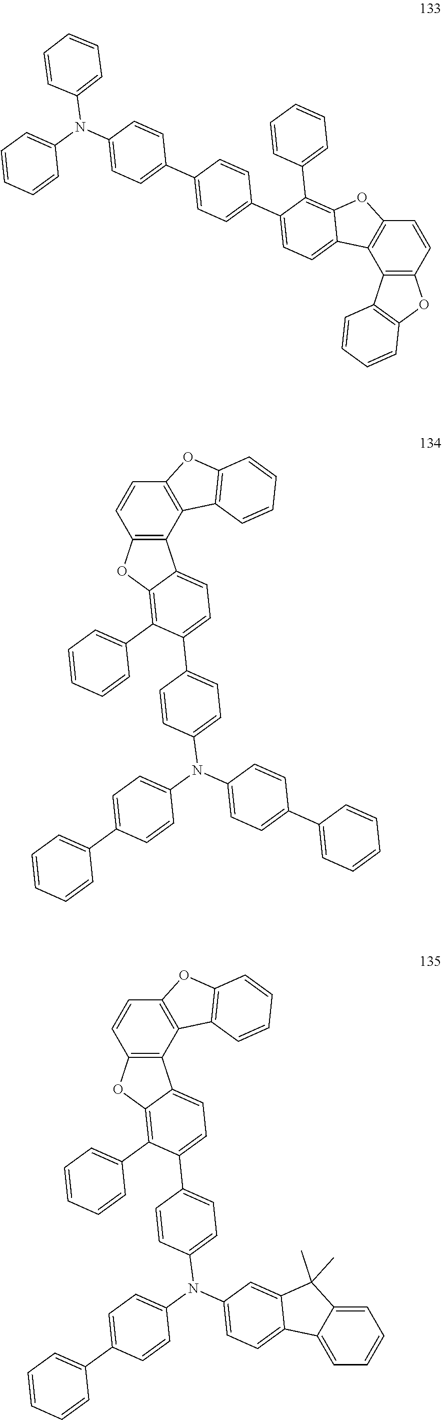 [SMILES: c1ccc(-c2c(-c3ccc(-c4ccc(N(c5ccccc5)c5ccccc5)cc4)cc3)ccc3c2oc2ccc4oc5ccccc5c4c23)cc1 ; c1ccc(-c2ccc(N(c3ccc(-c4ccccc4)cc3)c3ccc(-c4ccc5c(oc6ccc7oc8ccccc8c7c65)c4-c4ccccc4)cc3)cc2)cc1 ; CC1(C)c2ccccc2-c2ccc(N(c3ccc(-c4ccccc4)cc3)c3ccc(-c4ccc5c(oc6ccc7oc8ccccc8c7c65)c4-c4ccccc4)cc3)cc21]

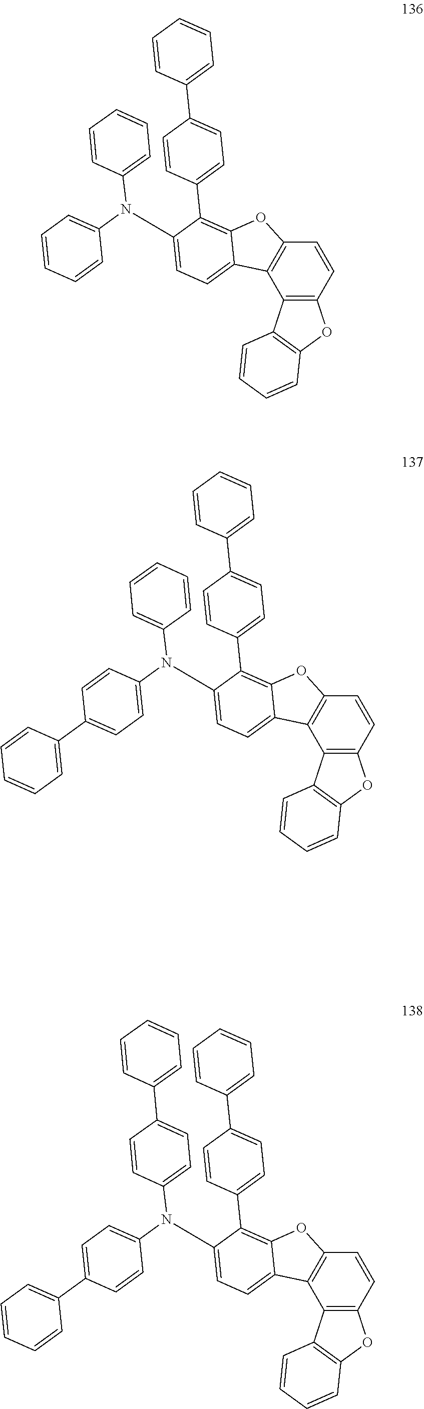 [SMILES: c1ccc(-c2ccc(-c3c(N(c4ccccc4)c4ccccc4)ccc4c3oc3ccc5oc6ccccc6c5c34)cc2)cc1 ; c1ccc(-c2ccc(-c3c(N(c4ccccc4)c4ccc(-c5ccccc5)cc4)ccc4c3oc3ccc5oc6ccccc6c5c34)cc2)cc1 ; c1ccc(-c2ccc(-c3c(N(c4ccc(-c5ccccc5)cc4)c4ccc(-c5ccccc5)cc4)ccc4c3oc3ccc5oc6ccccc6c5c34)cc2)cc1]

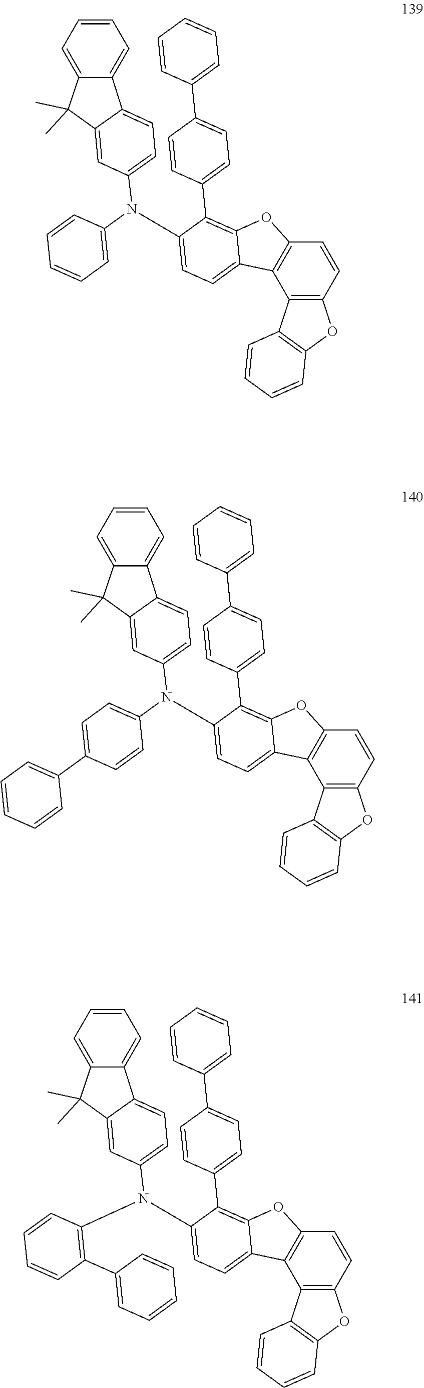 [SMILES: CC1(C)c2ccccc2-c2ccc(N(c3ccccc3)c3ccc4c(oc5ccc6oc7ccccc7c6c54)c3-c3ccc(-c4ccccc4)cc3)cc21 ; CC1(C)c2ccccc2-c2ccc(N(c3ccc(-c4ccccc4)cc3)c3ccc4c(oc5ccc6oc7ccccc7c6c54)c3-c3ccc(-c4ccccc4)cc3)cc21 ; CC1(C)c2ccccc2-c2ccc(N(c3ccccc3-c3ccccc3)c3ccc4c(oc5ccc6oc7ccccc7c6c54)c3-c3ccc(-c4ccccc4)cc3)cc21]

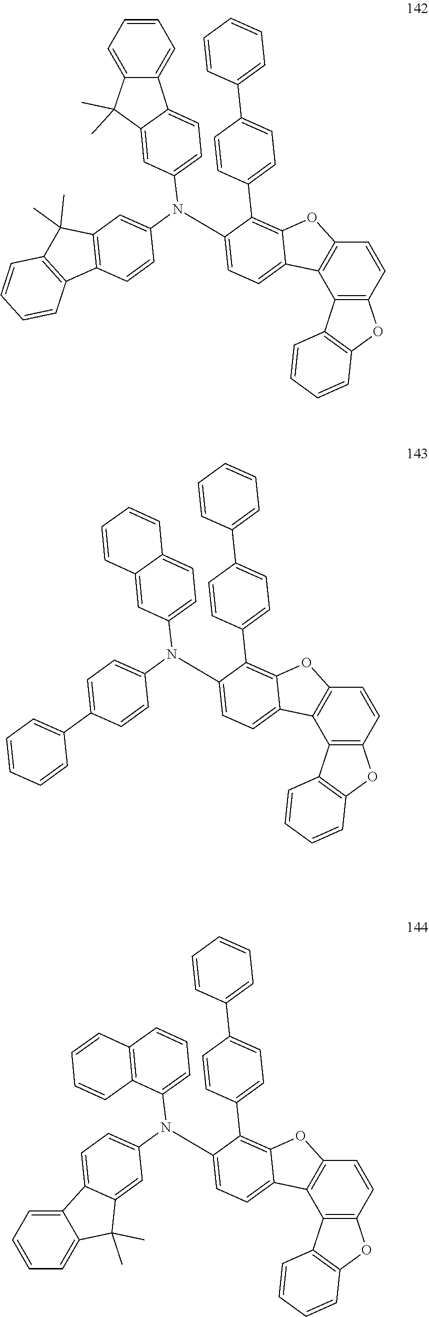 [SMILES: CC1(C)c2ccccc2-c2ccc(N(c3ccc4c(c3)C(C)(C)c3ccccc3-4)c3ccc4c(oc5ccc6oc7ccccc7c6c54)c3-c3ccc(-c4ccccc4)cc3)cc21 ; c1ccc(-c2ccc(-c3c(N(c4ccc(-c5ccccc5)cc4)c4ccc5ccccc5c4)ccc4c3oc3ccc5oc6ccccc6c5c34)cc2)cc1 ; CC1(C)c2ccccc2-c2ccc(N(c3ccc4c(oc5ccc6oc7ccccc7c6c54)c3-c3ccc(-c4ccccc4)cc3)c3cccc4ccccc34)cc21]

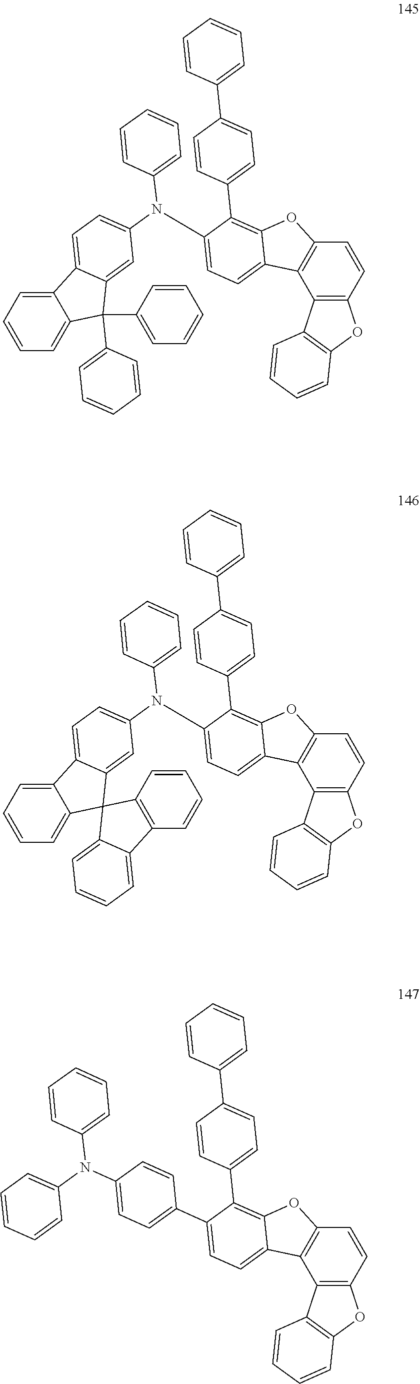 [SMILES: c1ccc(-c2ccc(-c3c(N(c4ccccc4)c4ccc5c(c4)C(c4ccccc4)(c4ccccc4)c4ccccc4-5)ccc4c3oc3ccc5oc6ccccc6c5c34)cc2)cc1 ; c1ccc(-c2ccc(-c3c(N(c4ccccc4)c4ccc5c(c4)C4(c6ccccc6-c6ccccc64)c4ccccc4-5)ccc4c3oc3ccc5oc6ccccc6c5c34)cc2)cc1 ; c1ccc(-c2ccc(-c3c(-c4ccc(N(c5ccccc5)c5ccccc5)cc4)ccc4c3oc3ccc5oc6ccccc6c5c34)cc2)cc1]

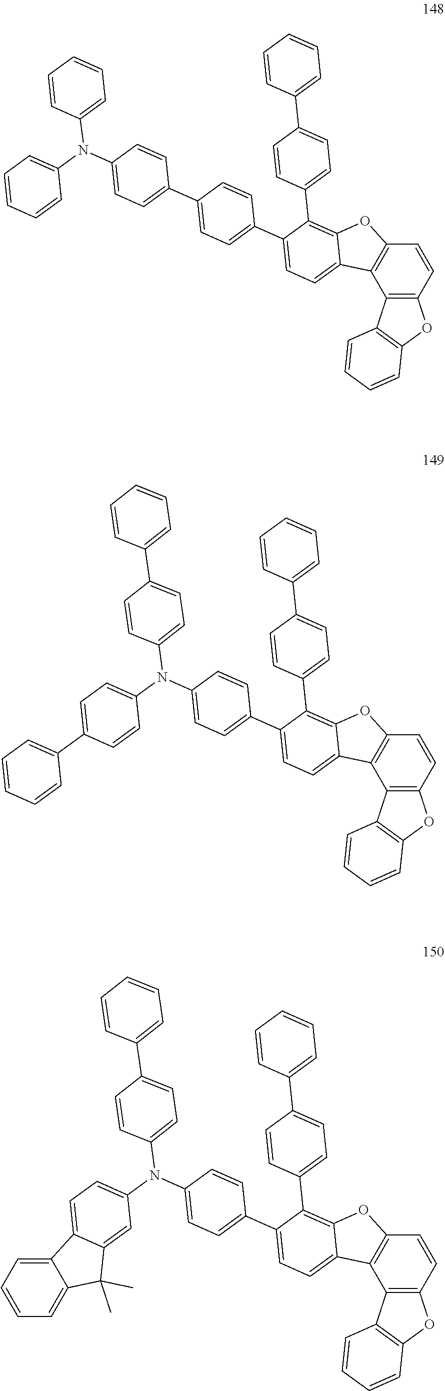 [SMILES: c1ccc(-c2ccc(-c3c(-c4ccc(-c5ccc(N(c6ccccc6)c6ccccc6)cc5)cc4)ccc4c3oc3ccc5oc6ccccc6c5c34)cc2)cc1 ; c1ccc(-c2ccc(-c3c(-c4ccc(N(c5ccc(-c6ccccc6)cc5)c5ccc(-c6ccccc6)cc5)cc4)ccc4c3oc3ccc5oc6ccccc6c5c34)cc2)cc1 ; CC1(C)c2ccccc2-c2ccc(N(c3ccc(-c4ccccc4)cc3)c3ccc(-c4ccc5c(oc6ccc7oc8ccccc8c7c65)c4-c4ccc(-c5ccccc5)cc4)cc3)cc21]

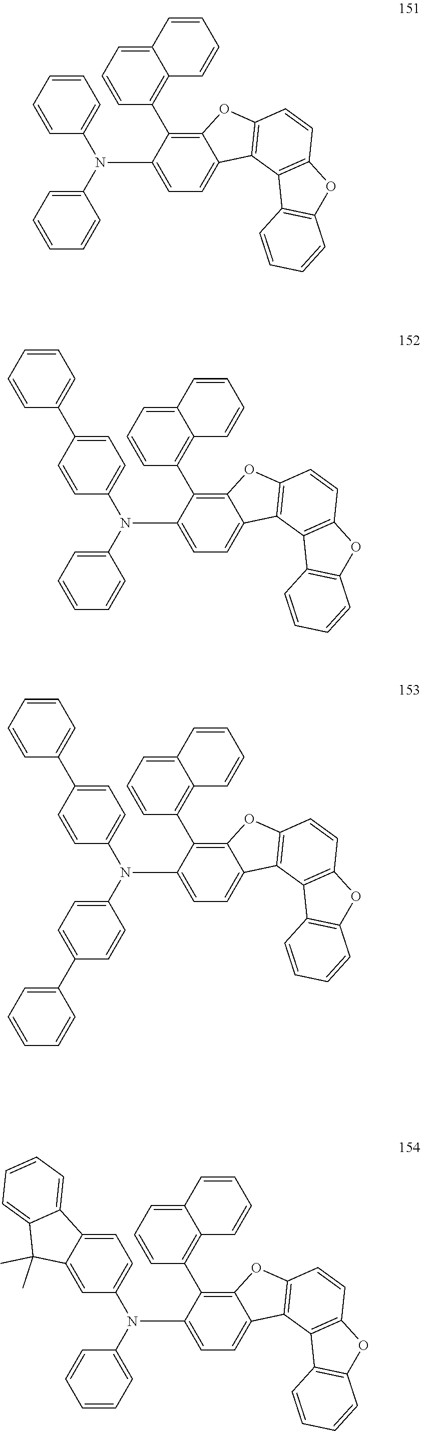 [SMILES: c1ccc(N(c2ccccc2)c2ccc3c(oc4ccc5oc6ccccc6c5c43)c2-c2cccc3ccccc23)cc1 ; c1ccc(-c2ccc(N(c3ccccc3)c3ccc4c(oc5ccc6oc7ccccc7c6c54)c3-c3cccc4ccccc34)cc2)cc1 ; c1ccc(-c2ccc(N(c3ccc(-c4ccccc4)cc3)c3ccc4c(oc5ccc6oc7ccccc7c6c54)c3-c3cccc4ccccc34)cc2)cc1 ; CC1(C)c2ccccc2-c2ccc(N(c3ccccc3)c3ccc4c(oc5ccc6oc7ccccc7c6c54)c3-c3cccc4ccccc34)cc21]

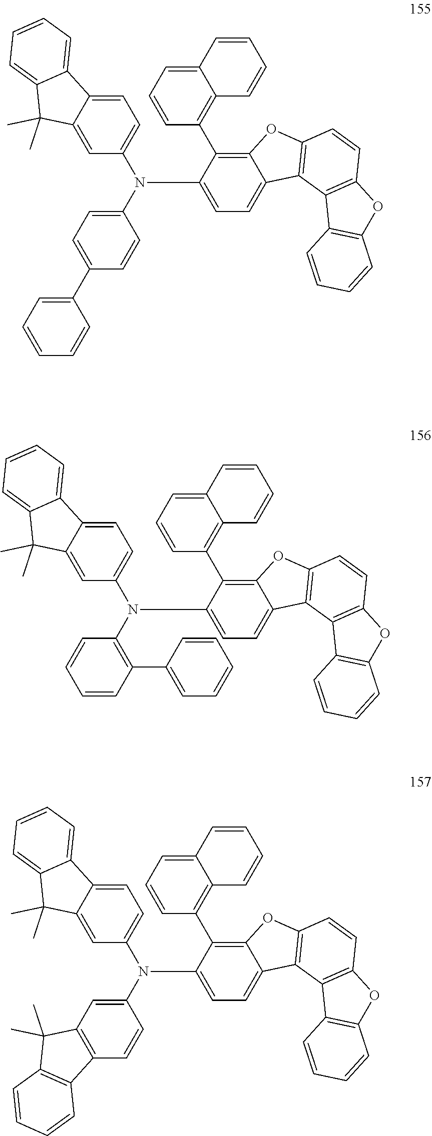 [SMILES: CC1(C)c2ccccc2-c2ccc(N(c3ccc(-c4ccccc4)cc3)c3ccc4c(oc5ccc6oc7ccccc7c6c54)c3-c3cccc4ccccc34)cc21 ; CC1(C)c2ccccc2-c2ccc(N(c3ccccc3-c3ccccc3)c3ccc4c(oc5ccc6oc7ccccc7c6c54)c3-c3cccc4ccccc34)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3cccc4ccccc34)cc21]

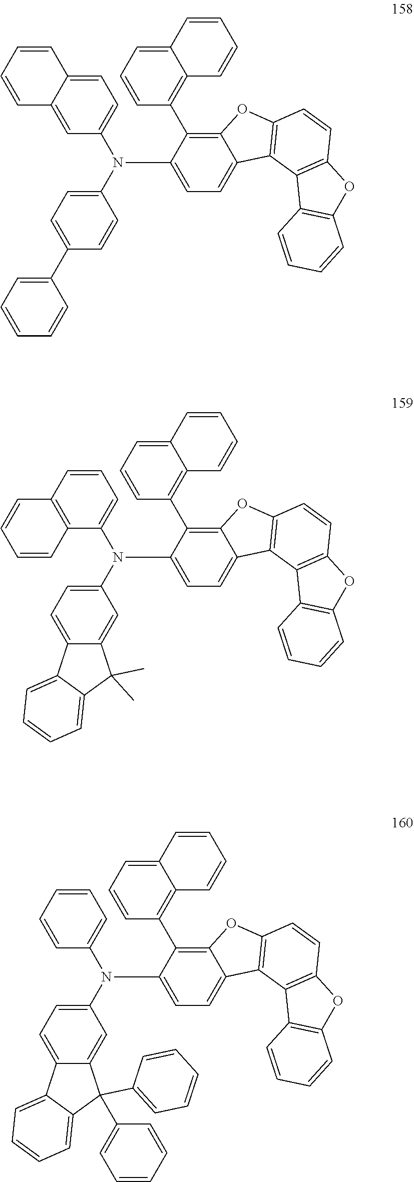 [SMILES: c1ccc(-c2ccc(N(c3ccc4ccccc4c3)c3ccc4c(oc5ccc6oc7ccccc7c6c54)c3-c3cccc4ccccc34)cc2)cc1 ; CC1(C)c2ccccc2-c2ccc(N(c3ccc4c(oc5ccc6oc7ccccc7c6c54)c3-c3cccc4ccccc34)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2cccc3ccccc23)cc1]

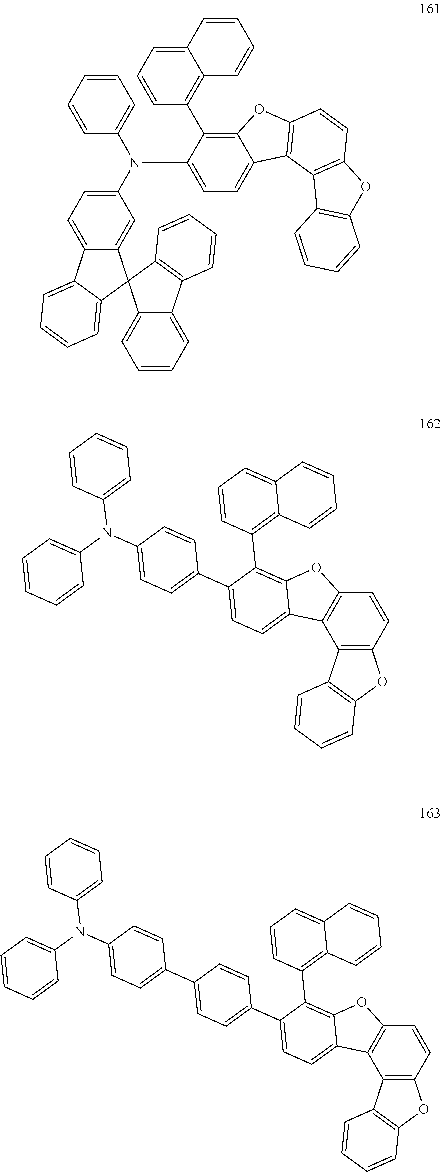 [SMILES: c1ccc(N(c2ccc3c(c2)C2(c4ccccc4-c4ccccc42)c2ccccc2-3)c2ccc3c(oc4ccc5oc6ccccc6c5c43)c2-c2cccc3ccccc23)cc1 ; c1ccc(N(c2ccccc2)c2ccc(-c3ccc4c(oc5ccc6oc7ccccc7c6c54)c3-c3cccc4ccccc34)cc2)cc1 ; c1ccc(N(c2ccccc2)c2ccc(-c3ccc(-c4ccc5c(oc6ccc7oc8ccccc8c7c65)c4-c4cccc5ccccc45)cc3)cc2)cc1]

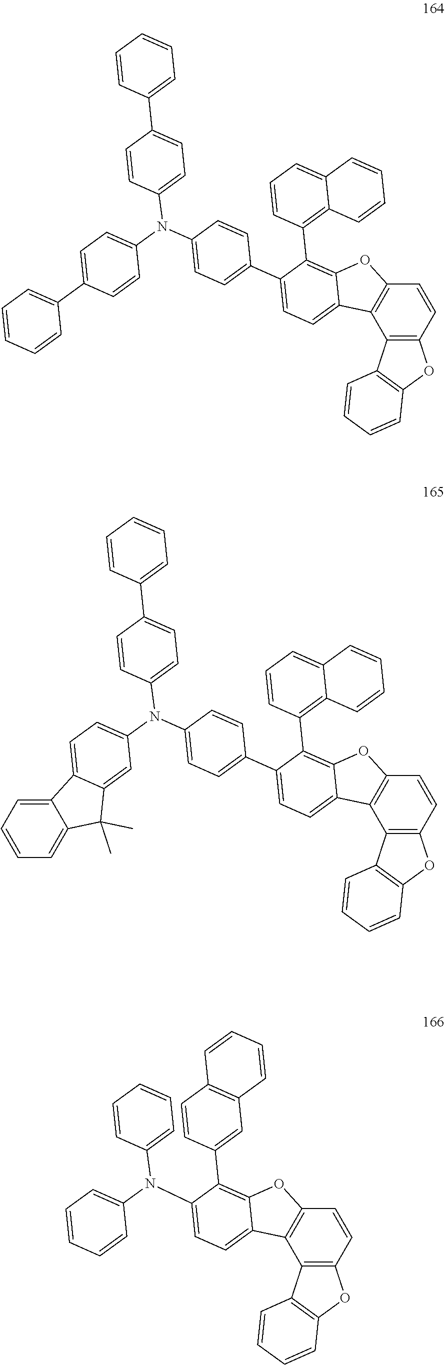 [SMILES: c1ccc(-c2ccc(N(c3ccc(-c4ccccc4)cc3)c3ccc(-c4ccc5c(oc6ccc7oc8ccccc8c7c65)c4-c4cccc5ccccc45)cc3)cc2)cc1 ; CC1(C)c2ccccc2-c2ccc(N(c3ccc(-c4ccccc4)cc3)c3ccc(-c4ccc5c(oc6ccc7oc8ccccc8c7c65)c4-c4cccc5ccccc45)cc3)cc21 ; c1ccc(N(c2ccccc2)c2ccc3c(oc4ccc5oc6ccccc6c5c43)c2-c2ccc3ccccc3c2)cc1]

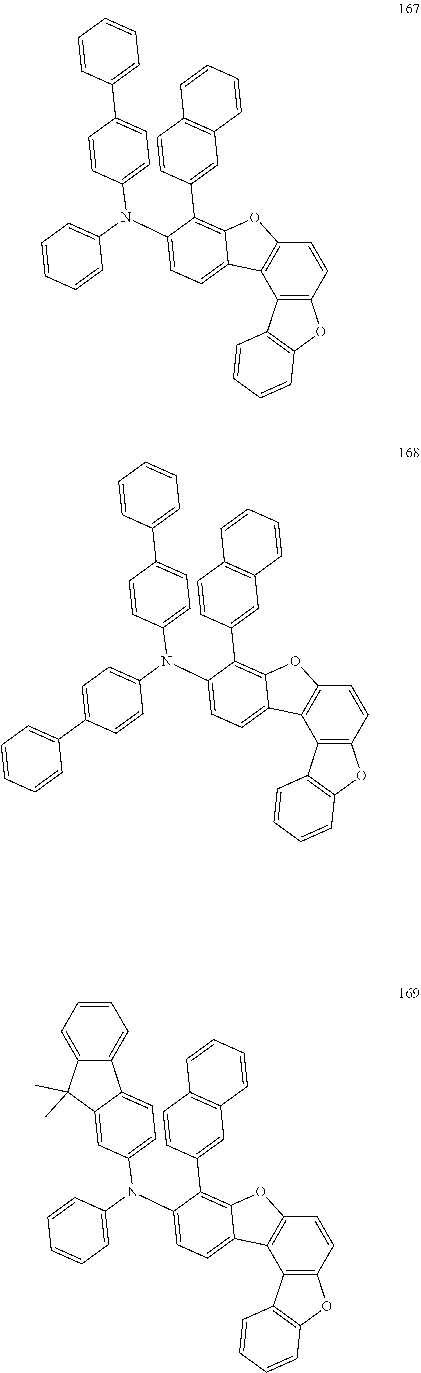 [SMILES: c1ccc(-c2ccc(N(c3ccccc3)c3ccc4c(oc5ccc6oc7ccccc7c6c54)c3-c3ccc4ccccc4c3)cc2)cc1 ; c1ccc(-c2ccc(N(c3ccc(-c4ccccc4)cc3)c3ccc4c(oc5ccc6oc7ccccc7c6c54)c3-c3ccc4ccccc4c3)cc2)cc1 ; CC1(C)c2ccccc2-c2ccc(N(c3ccccc3)c3ccc4c(oc5ccc6oc7ccccc7c6c54)c3-c3ccc4ccccc4c3)cc21]

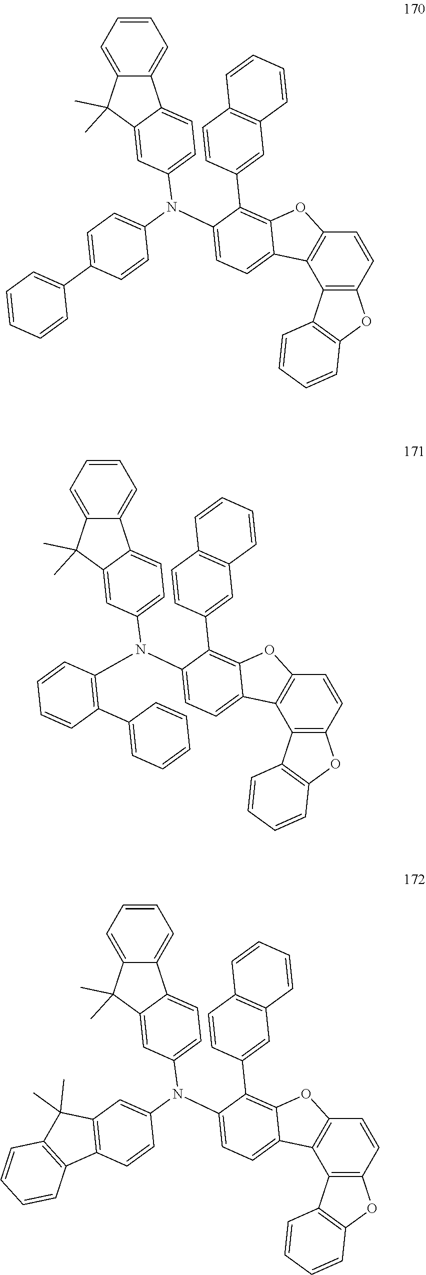 [SMILES: CC1(C)c2ccccc2-c2ccc(N(c3ccc(-c4ccccc4)cc3)c3ccc4c(oc5ccc6oc7ccccc7c6c54)c3-c3ccc4ccccc4c3)cc21 ; CC1(C)c2ccccc2-c2ccc(N(c3ccccc3-c3ccccc3)c3ccc4c(oc5ccc6oc7ccccc7c6c54)c3-c3ccc4ccccc4c3)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3ccc4ccccc4c3)cc21]

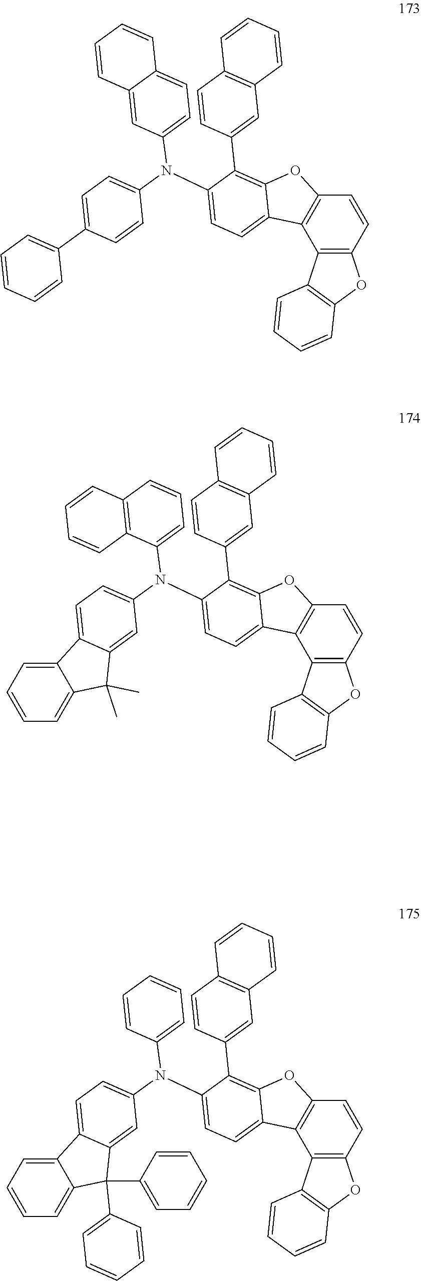 [SMILES: c1ccc(-c2ccc(N(c3ccc4ccccc4c3)c3ccc4c(oc5ccc6oc7ccccc7c6c54)c3-c3ccc4ccccc4c3)cc2)cc1 ; CC1(C)c2ccccc2-c2ccc(N(c3ccc4c(oc5ccc6oc7ccccc7c6c54)c3-c3ccc4ccccc4c3)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2ccc3ccccc3c2)cc1]

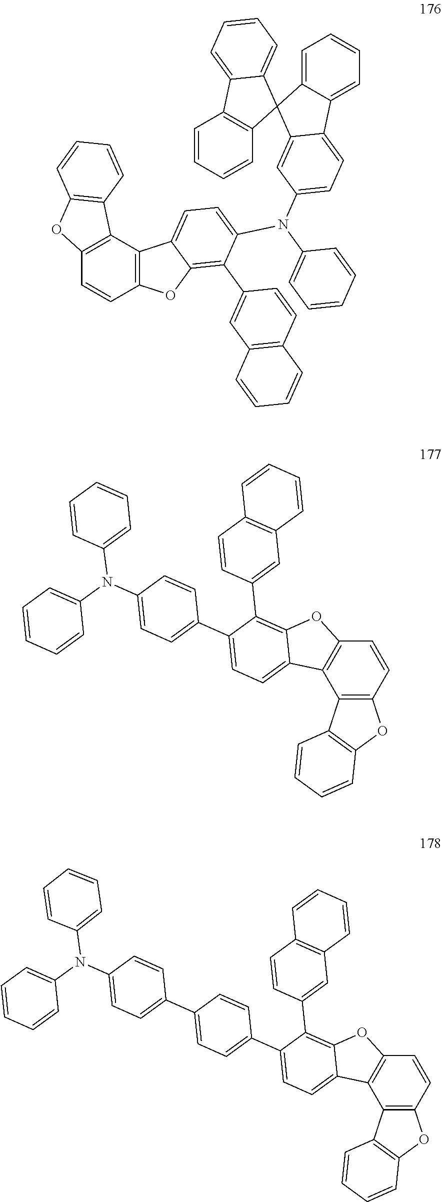 [SMILES: c1ccc(N(c2ccc3c(c2)C2(c4ccccc4-c4ccccc42)c2ccccc2-3)c2ccc3c(oc4ccc5oc6ccccc6c5c43)c2-c2ccc3ccccc3c2)cc1 ; c1ccc(N(c2ccccc2)c2ccc(-c3ccc4c(oc5ccc6oc7ccccc7c6c54)c3-c3ccc4ccccc4c3)cc2)cc1 ; c1ccc(N(c2ccccc2)c2ccc(-c3ccc(-c4ccc5c(oc6ccc7oc8ccccc8c7c65)c4-c4ccc5ccccc5c4)cc3)cc2)cc1]

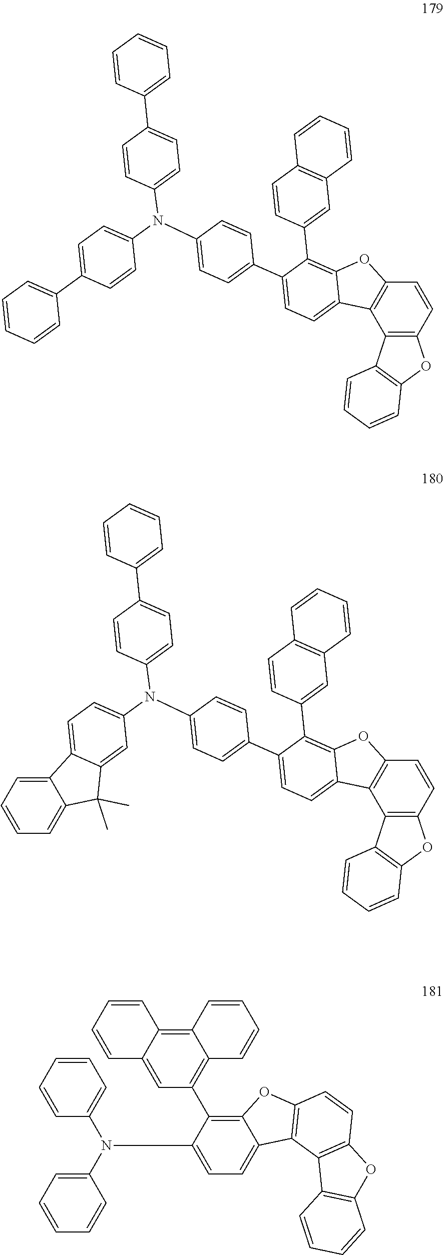 [SMILES: c1ccc(-c2ccc(N(c3ccc(-c4ccccc4)cc3)c3ccc(-c4ccc5c(oc6ccc7oc8ccccc8c7c65)c4-c4ccc5ccccc5c4)cc3)cc2)cc1 ; CC1(C)c2ccccc2-c2ccc(N(c3ccc(-c4ccccc4)cc3)c3ccc(-c4ccc5c(oc6ccc7oc8ccccc8c7c65)c4-c4ccc5ccccc5c4)cc3)cc21 ; c1ccc(N(c2ccccc2)c2ccc3c(oc4ccc5oc6ccccc6c5c43)c2-c2cc3ccccc3c3ccccc23)cc1]

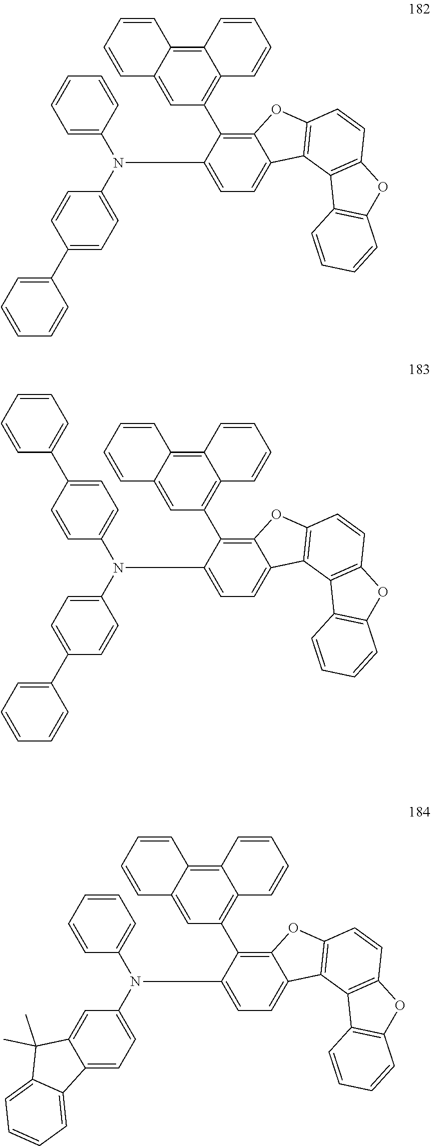 [SMILES: c1ccc(-c2ccc(N(c3ccccc3)c3ccc4c(oc5ccc6oc7ccccc7c6c54)c3-c3cc4ccccc4c4ccccc34)cc2)cc1 ; c1ccc(-c2ccc(N(c3ccc(-c4ccccc4)cc3)c3ccc4c(oc5ccc6oc7ccccc7c6c54)c3-c3cc4ccccc4c4ccccc34)cc2)cc1 ; CC1(C)c2ccccc2-c2ccc(N(c3ccccc3)c3ccc4c(oc5ccc6oc7ccccc7c6c54)c3-c3cc4ccccc4c4ccccc34)cc21]

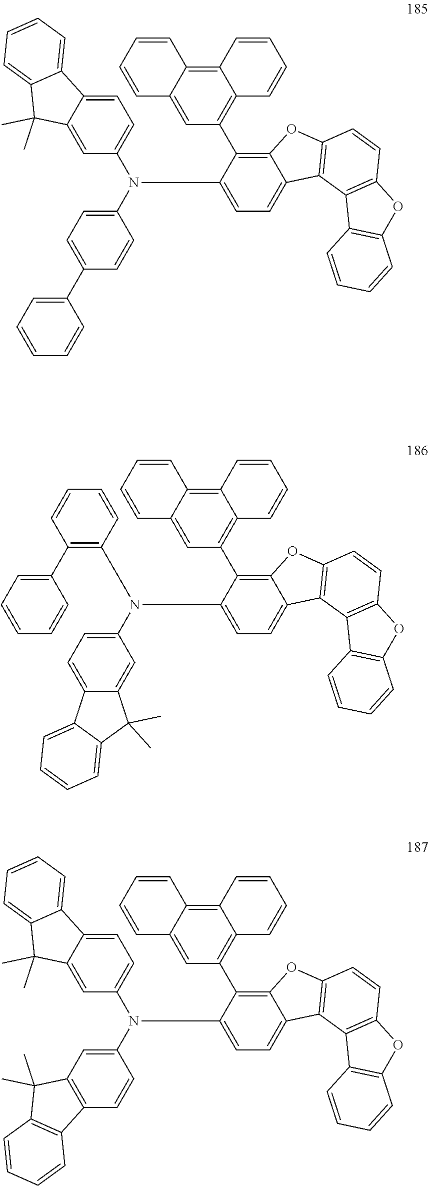 [SMILES: CC1(C)c2ccccc2-c2ccc(N(c3ccc(-c4ccccc4)cc3)c3ccc4c(oc5ccc6oc7ccccc7c6c54)c3-c3cc4ccccc4c4ccccc34)cc21 ; CC1(C)c2ccccc2-c2ccc(N(c3ccccc3-c3ccccc3)c3ccc4c(oc5ccc6oc7ccccc7c6c54)c3-c3cc4ccccc4c4ccccc34)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3cc4ccccc4c4ccccc34)cc21]

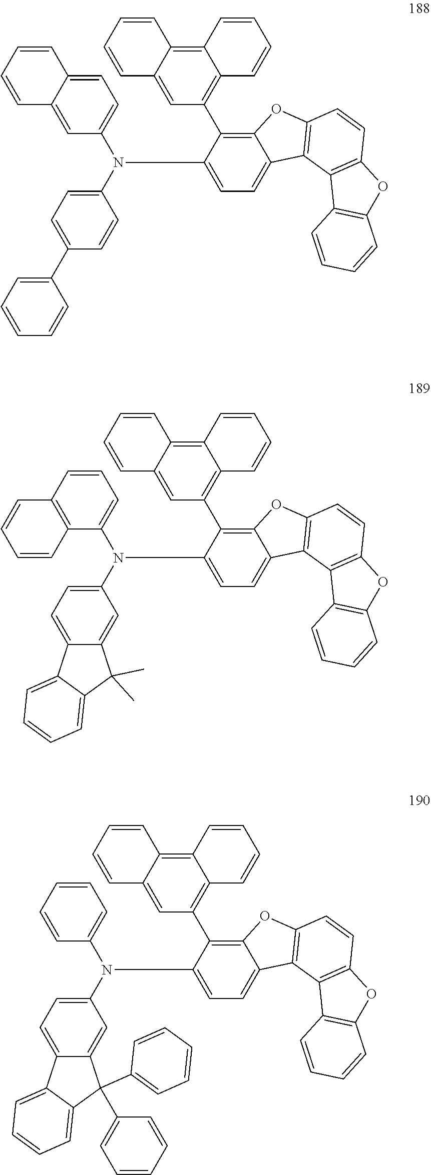 [SMILES: c1ccc(-c2ccc(N(c3ccc4ccccc4c3)c3ccc4c(oc5ccc6oc7ccccc7c6c54)c3-c3cc4ccccc4c4ccccc34)cc2)cc1 ; CC1(C)c2ccccc2-c2ccc(N(c3ccc4c(oc5ccc6oc7ccccc7c6c54)c3-c3cc4ccccc4c4ccccc34)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2cc3ccccc3c3ccccc23)cc1]

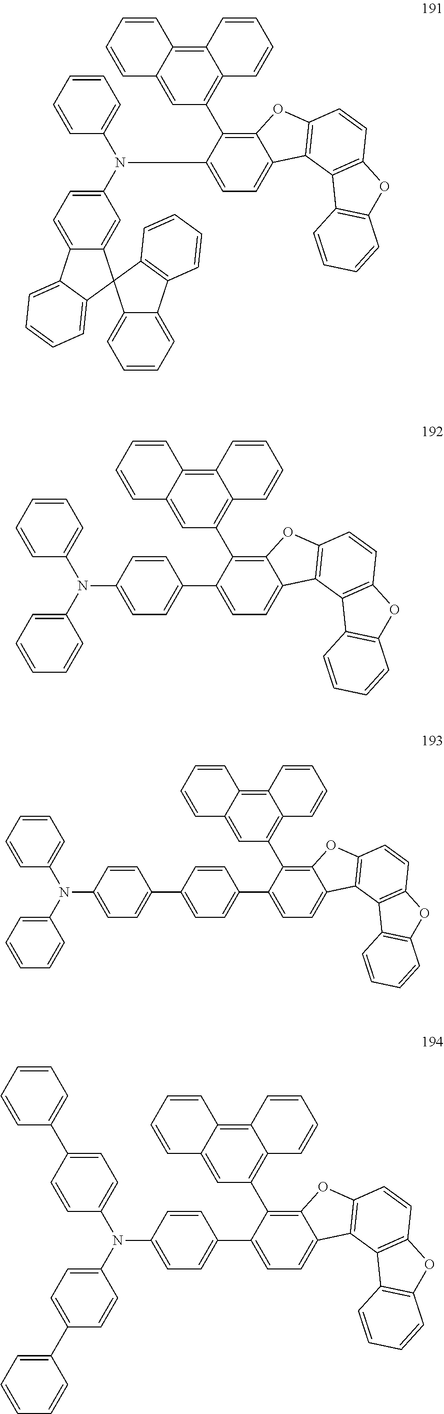 [SMILES: c1ccc(N(c2ccc3c(c2)C2(c4ccccc4-c4ccccc42)c2ccccc2-3)c2ccc3c(oc4ccc5oc6ccccc6c5c43)c2-c2cc3ccccc3c3ccccc23)cc1 ; c1ccc(N(c2ccccc2)c2ccc(-c3ccc4c(oc5ccc6oc7ccccc7c6c54)c3-c3cc4ccccc4c4ccccc34)cc2)cc1 ; c1ccc(N(c2ccccc2)c2ccc(-c3ccc(-c4ccc5c(oc6ccc7oc8ccccc8c7c65)c4-c4cc5ccccc5c5ccccc45)cc3)cc2)cc1 ; c1ccc(-c2ccc(N(c3ccc(-c4ccccc4)cc3)c3ccc(-c4ccc5c(oc6ccc7oc8ccccc8c7c65)c4-c4cc5ccccc5c5ccccc45)cc3)cc2)cc1]

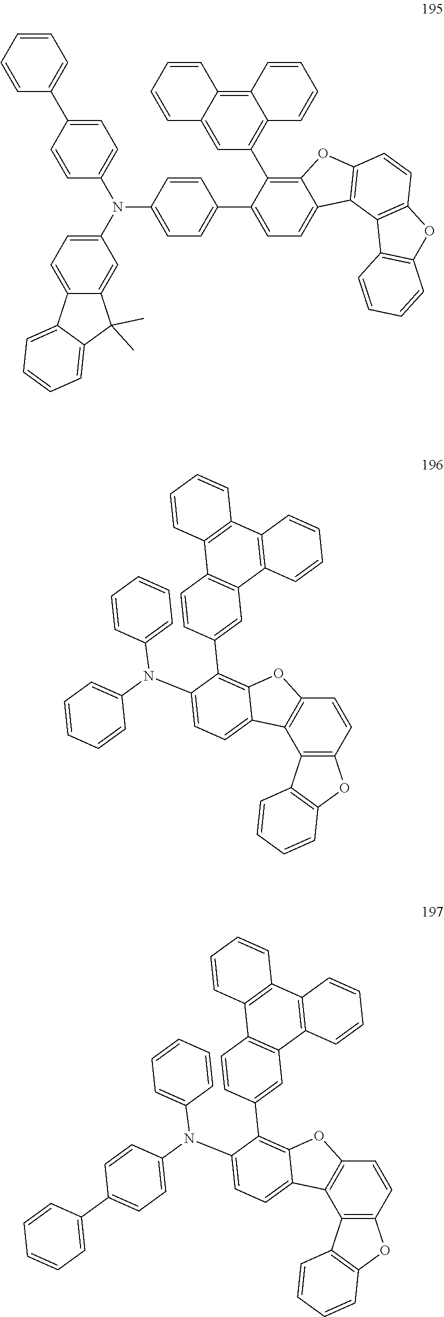 [SMILES: CC1(C)c2ccccc2-c2ccc(N(c3ccc(-c4ccccc4)cc3)c3ccc(-c4ccc5c(oc6ccc7oc8ccccc8c7c65)c4-c4cc5ccccc5c5ccccc45)cc3)cc21 ; c1ccc(N(c2ccccc2)c2ccc3c(oc4ccc5oc6ccccc6c5c43)c2-c2ccc3c4ccccc4c4ccccc4c3c2)cc1 ; c1ccc(-c2ccc(N(c3ccccc3)c3ccc4c(oc5ccc6oc7ccccc7c6c54)c3-c3ccc4c5ccccc5c5ccccc5c4c3)cc2)cc1]

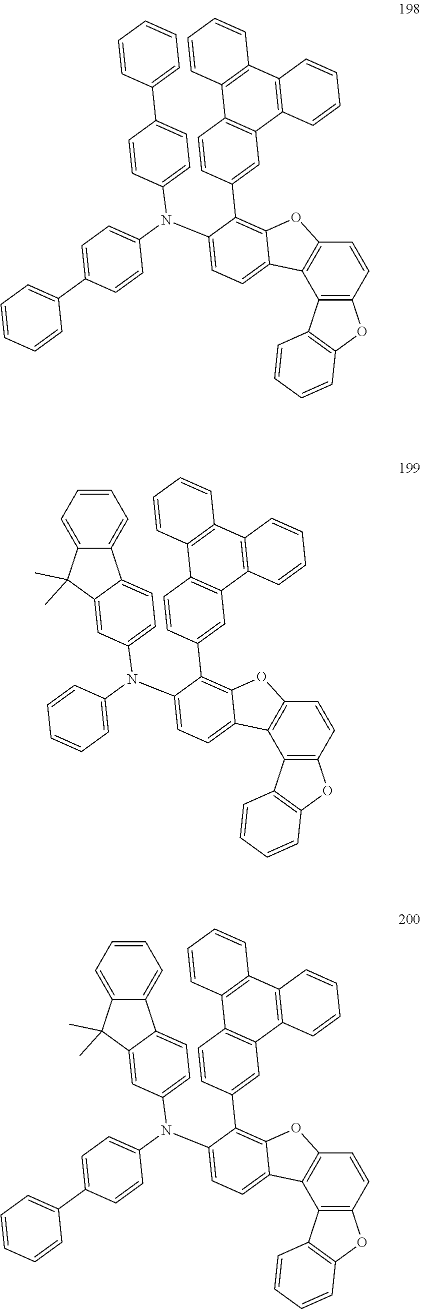 [SMILES: c1ccc(-c2ccc(N(c3ccc(-c4ccccc4)cc3)c3ccc4c(oc5ccc6oc7ccccc7c6c54)c3-c3ccc4c5ccccc5c5ccccc5c4c3)cc2)cc1 ; CC1(C)c2ccccc2-c2ccc(N(c3ccccc3)c3ccc4c(oc5ccc6oc7ccccc7c6c54)c3-c3ccc4c5ccccc5c5ccccc5c4c3)cc21 ; CC1(C)c2ccccc2-c2ccc(N(c3ccc(-c4ccccc4)cc3)c3ccc4c(oc5ccc6oc7ccccc7c6c54)c3-c3ccc4c5ccccc5c5ccccc5c4c3)cc21]

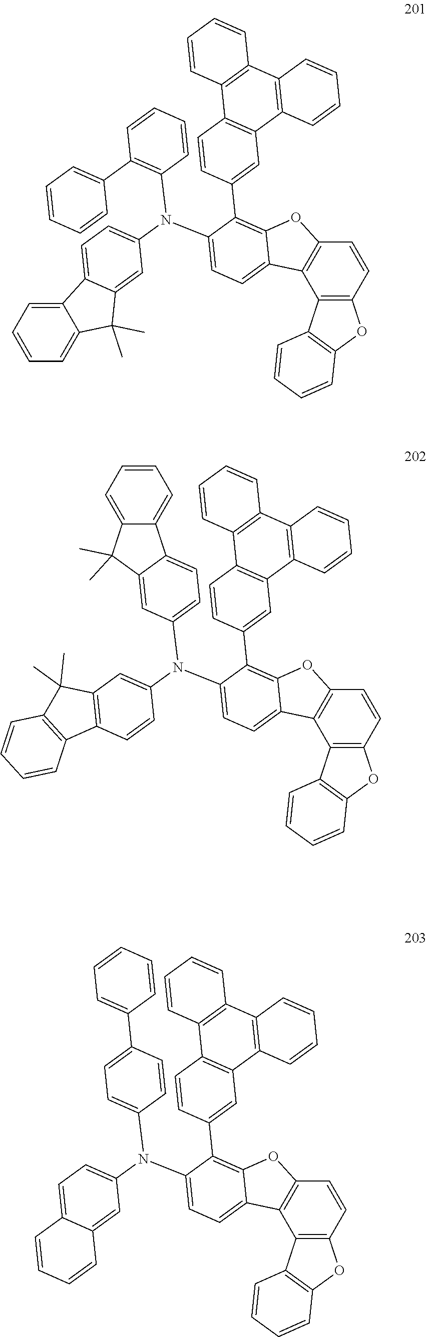 [SMILES: CC1(C)c2ccccc2-c2ccc(N(c3ccccc3-c3ccccc3)c3ccc4c(oc5ccc6oc7ccccc7c6c54)c3-c3ccc4c5ccccc5c5ccccc5c4c3)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3ccc4c5ccccc5c5ccccc5c4c3)cc21 ; c1ccc(-c2ccc(N(c3ccc4ccccc4c3)c3ccc4c(oc5ccc6oc7ccccc7c6c54)c3-c3ccc4c5ccccc5c5ccccc5c4c3)cc2)cc1]

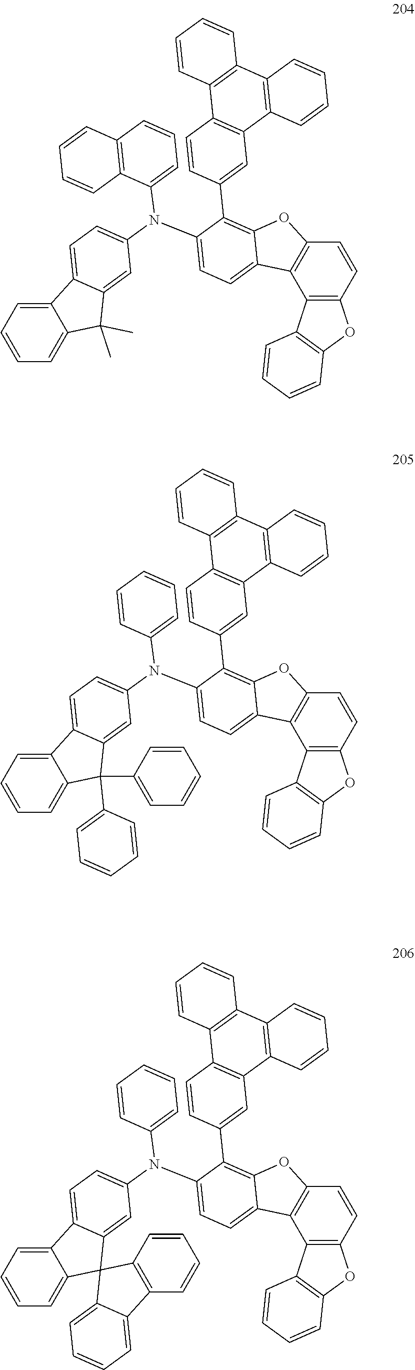 [SMILES: CC1(C)c2ccccc2-c2ccc(N(c3ccc4c(oc5ccc6oc7ccccc7c6c54)c3-c3ccc4c5ccccc5c5ccccc5c4c3)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2ccc3c4ccccc4c4ccccc4c3c2)cc1 ; c1ccc(N(c2ccc3c(c2)C2(c4ccccc4-c4ccccc42)c2ccccc2-3)c2ccc3c(oc4ccc5oc6ccccc6c5c43)c2-c2ccc3c4ccccc4c4ccccc4c3c2)cc1]

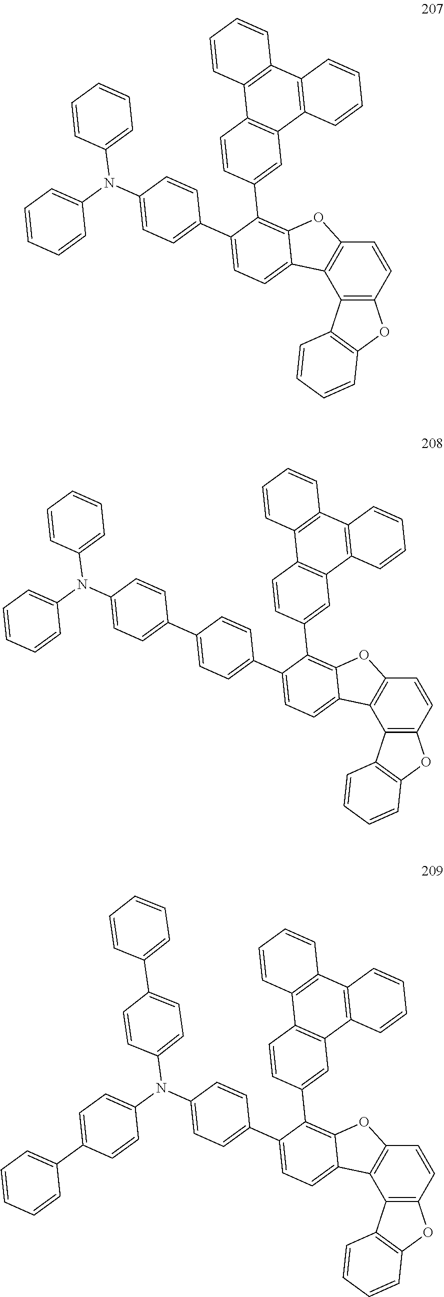 [SMILES: c1ccc(N(c2ccccc2)c2ccc(-c3ccc4c(oc5ccc6oc7ccccc7c6c54)c3-c3ccc4c5ccccc5c5ccccc5c4c3)cc2)cc1 ; c1ccc(N(c2ccccc2)c2ccc(-c3ccc(-c4ccc5c(oc6ccc7oc8ccccc8c7c65)c4-c4ccc5c6ccccc6c6ccccc6c5c4)cc3)cc2)cc1 ; c1ccc(-c2ccc(N(c3ccc(-c4ccccc4)cc3)c3ccc(-c4ccc5c(oc6ccc7oc8ccccc8c7c65)c4-c4ccc5c6ccccc6c6ccccc6c5c4)cc3)cc2)cc1]

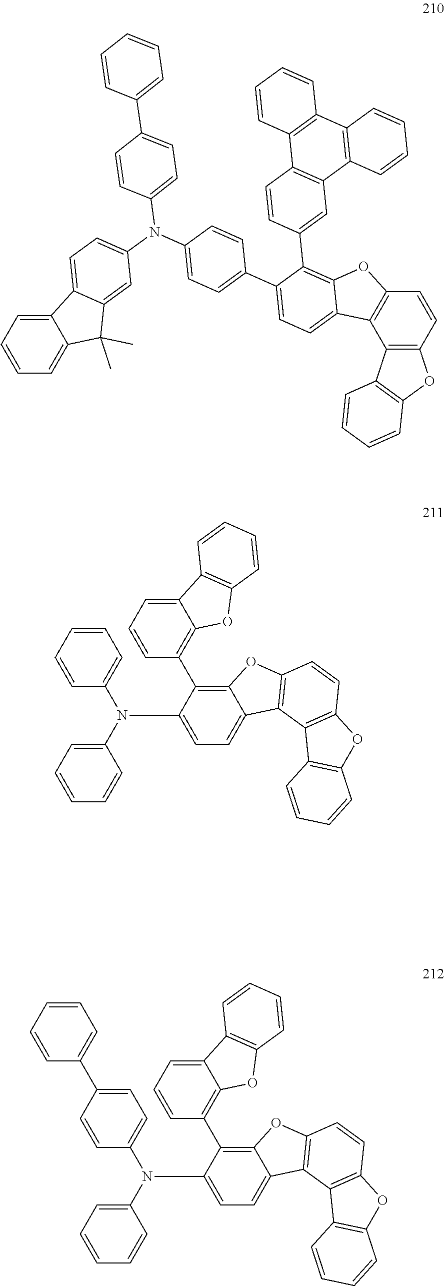 [SMILES: CC1(C)c2ccccc2-c2ccc(N(c3ccc(-c4ccccc4)cc3)c3ccc(-c4ccc5c(oc6ccc7oc8ccccc8c7c65)c4-c4ccc5c6ccccc6c6ccccc6c5c4)cc3)cc21 ; c1ccc(N(c2ccccc2)c2ccc3c(oc4ccc5oc6ccccc6c5c43)c2-c2cccc3c2oc2ccccc23)cc1 ; c1ccc(-c2ccc(N(c3ccccc3)c3ccc4c(oc5ccc6oc7ccccc7c6c54)c3-c3cccc4c3oc3ccccc34)cc2)cc1]

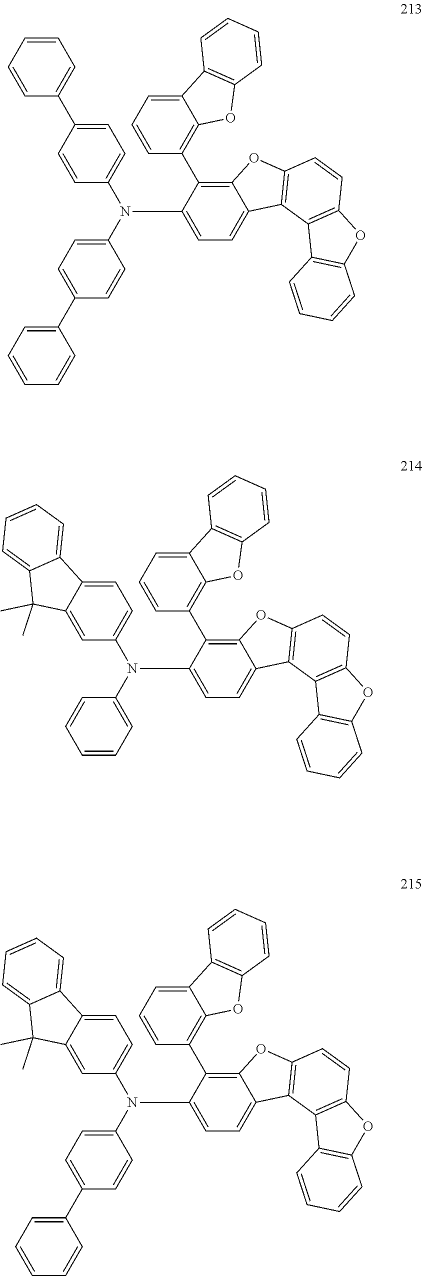 [SMILES: c1ccc(-c2ccc(N(c3ccc(-c4ccccc4)cc3)c3ccc4c(oc5ccc6oc7ccccc7c6c54)c3-c3cccc4c3oc3ccccc34)cc2)cc1 ; CC1(C)c2ccccc2-c2ccc(N(c3ccccc3)c3ccc4c(oc5ccc6oc7ccccc7c6c54)c3-c3cccc4c3oc3ccccc34)cc21 ; CC1(C)c2ccccc2-c2ccc(N(c3ccc(-c4ccccc4)cc3)c3ccc4c(oc5ccc6oc7ccccc7c6c54)c3-c3cccc4c3oc3ccccc34)cc21]

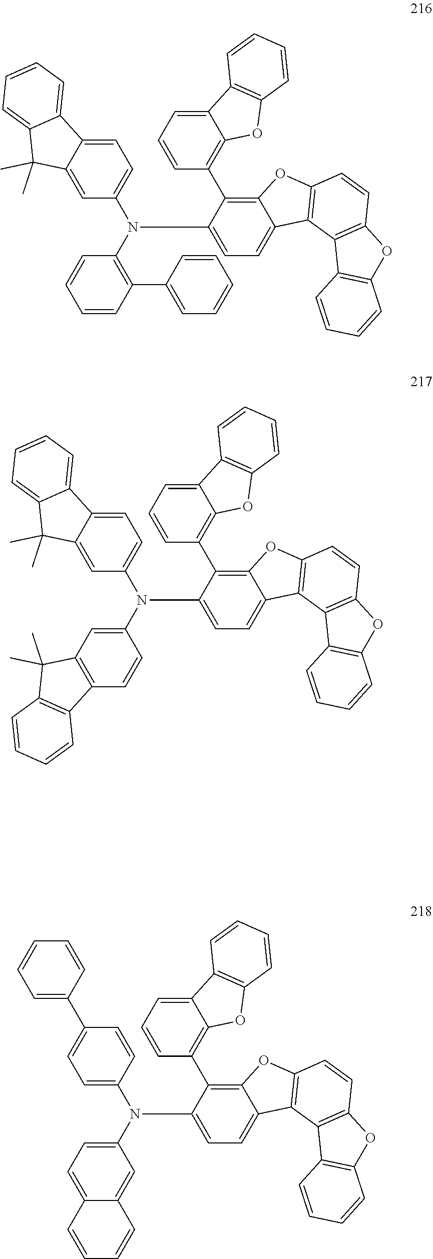 [SMILES: CC1(C)c2ccccc2-c2ccc(N(c3ccccc3-c3ccccc3)c3ccc4c(oc5ccc6oc7ccccc7c6c54)c3-c3cccc4c3oc3ccccc34)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3cccc4c3oc3ccccc34)cc21 ; c1ccc(-c2ccc(N(c3ccc4ccccc4c3)c3ccc4c(oc5ccc6oc7ccccc7c6c54)c3-c3cccc4c3oc3ccccc34)cc2)cc1]

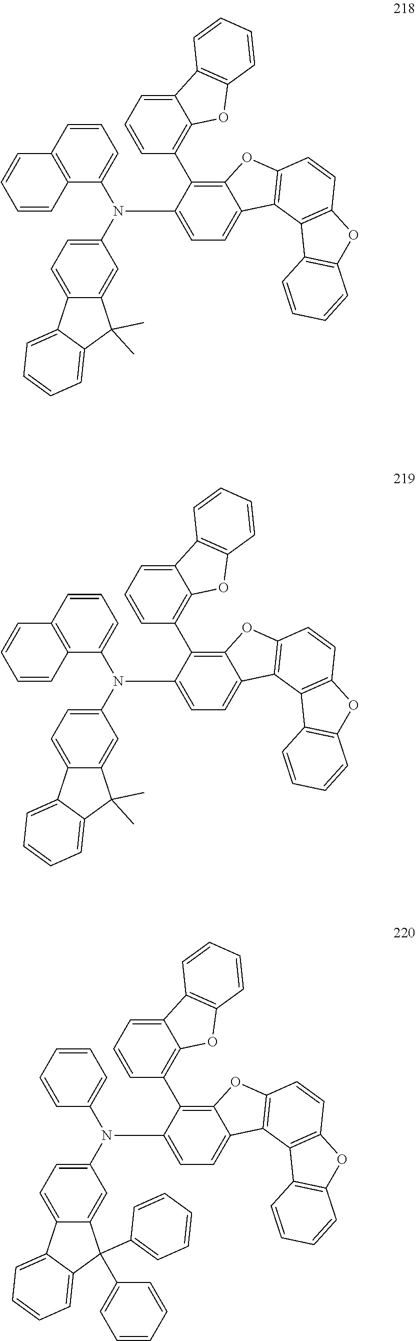 [SMILES: CC1(C)c2ccccc2-c2ccc(N(c3ccc4c(oc5ccc6oc7ccccc7c6c54)c3-c3cccc4c3oc3ccccc34)c3cccc4ccccc34)cc21 ; CC1(C)c2ccccc2-c2ccc(N(c3ccc4c(oc5ccc6oc7ccccc7c6c54)c3-c3cccc4c3oc3ccccc34)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2cccc3c2oc2ccccc23)cc1]

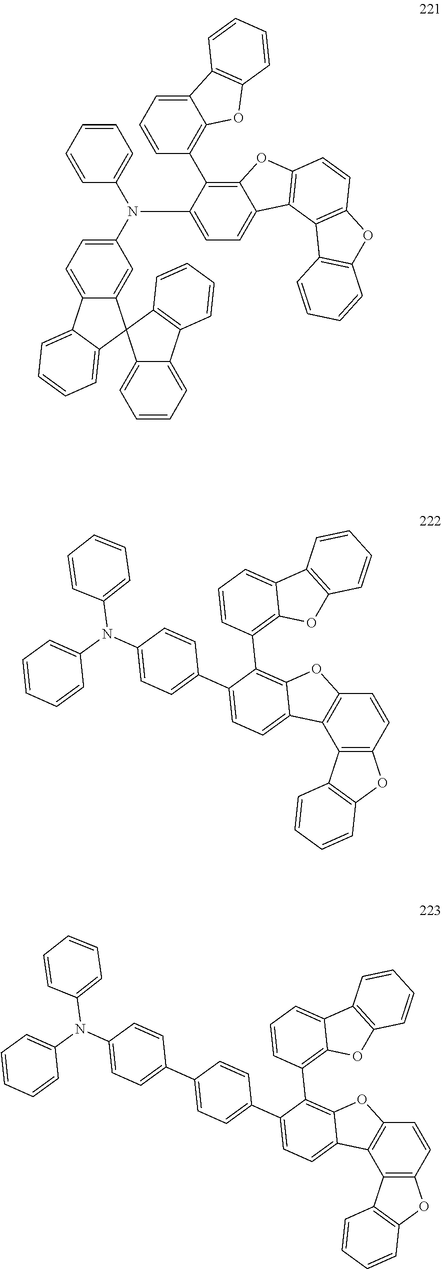 [SMILES: c1ccc(N(c2ccc3c(c2)C2(c4ccccc4-c4ccccc42)c2ccccc2-3)c2ccc3c(oc4ccc5oc6ccccc6c5c43)c2-c2cccc3c2oc2ccccc23)cc1 ; c1ccc(N(c2ccccc2)c2ccc(-c3ccc4c(oc5ccc6oc7ccccc7c6c54)c3-c3cccc4c3oc3ccccc34)cc2)cc1 ; c1ccc(N(c2ccccc2)c2ccc(-c3ccc(-c4ccc5c(oc6ccc7oc8ccccc8c7c65)c4-c4cccc5c4oc4ccccc45)cc3)cc2)cc1]

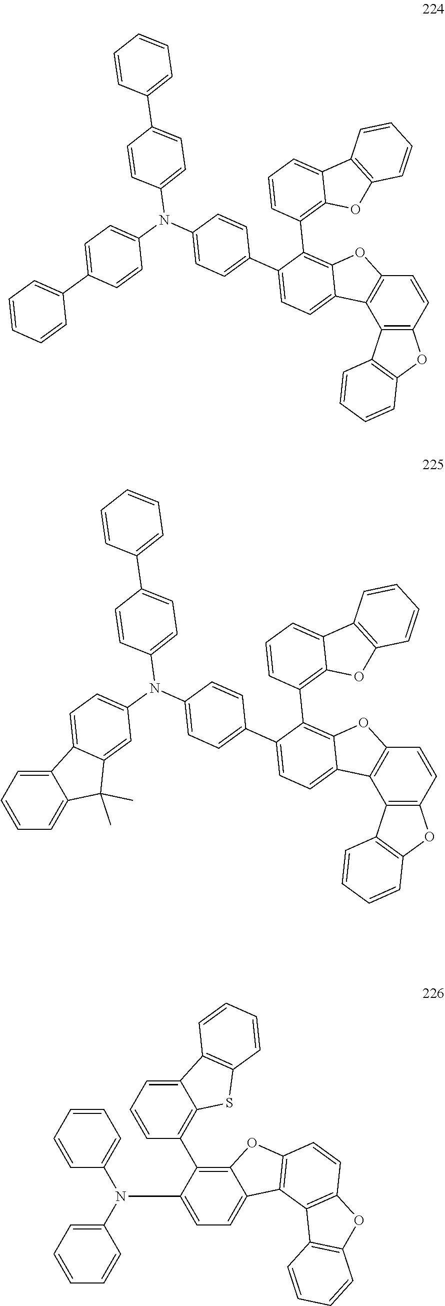 [SMILES: c1ccc(-c2ccc(N(c3ccc(-c4ccccc4)cc3)c3ccc(-c4ccc5c(oc6ccc7oc8ccccc8c7c65)c4-c4cccc5c4oc4ccccc45)cc3)cc2)cc1 ; CC1(C)c2ccccc2-c2ccc(N(c3ccc(-c4ccccc4)cc3)c3ccc(-c4ccc5c(oc6ccc7oc8ccccc8c7c65)c4-c4cccc5c4oc4ccccc45)cc3)cc21 ; c1ccc(N(c2ccccc2)c2ccc3c(oc4ccc5oc6ccccc6c5c43)c2-c2cccc3c2sc2ccccc23)cc1]

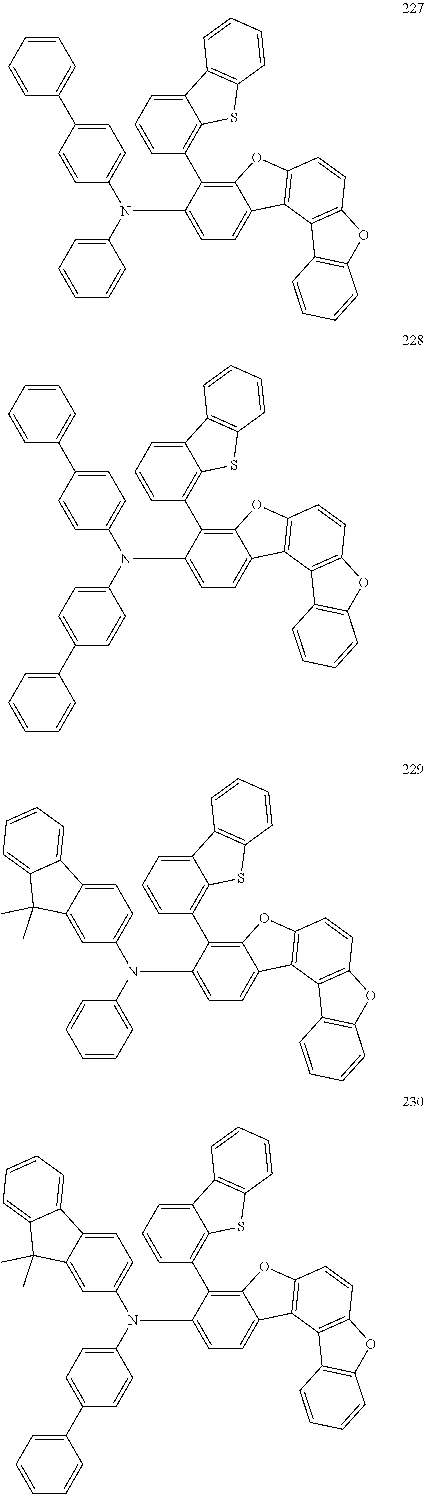 [SMILES: c1ccc(-c2ccc(N(c3ccccc3)c3ccc4c(oc5ccc6oc7ccccc7c6c54)c3-c3cccc4c3sc3ccccc34)cc2)cc1 ; c1ccc(-c2ccc(N(c3ccc(-c4ccccc4)cc3)c3ccc4c(oc5ccc6oc7ccccc7c6c54)c3-c3cccc4c3sc3ccccc34)cc2)cc1 ; CC1(C)c2ccccc2-c2ccc(N(c3ccccc3)c3ccc4c(oc5ccc6oc7ccccc7c6c54)c3-c3cccc4c3sc3ccccc34)cc21 ; CC1(C)c2ccccc2-c2ccc(N(c3ccc(-c4ccccc4)cc3)c3ccc4c(oc5ccc6oc7ccccc7c6c54)c3-c3cccc4c3sc3ccccc34)cc21]

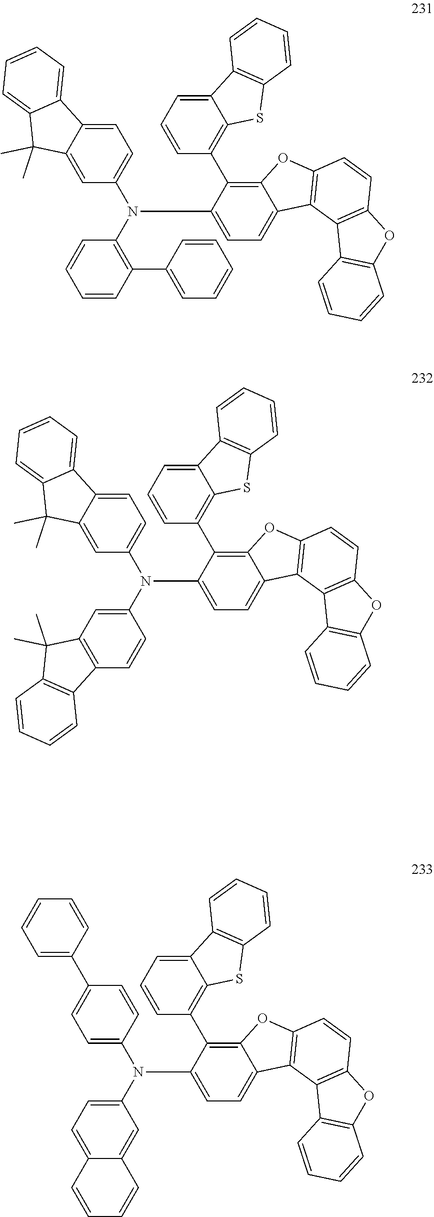 [SMILES: CC1(C)c2ccccc2-c2ccc(N(c3ccccc3-c3ccccc3)c3ccc4c(oc5ccc6oc7ccccc7c6c54)c3-c3cccc4c3sc3ccccc34)cc21 ; CC1(C)c2ccccc2-c2ccc(N(c3ccc4c(c3)C(C)(C)c3ccccc3-4)c3ccc4c(oc5ccc6oc7ccccc7c6c54)c3-c3cccc4c3sc3ccccc34)cc21 ; c1ccc(-c2ccc(N(c3ccc4ccccc4c3)c3ccc4c(oc5ccc6oc7ccccc7c6c54)c3-c3cccc4c3sc3ccccc34)cc2)cc1]

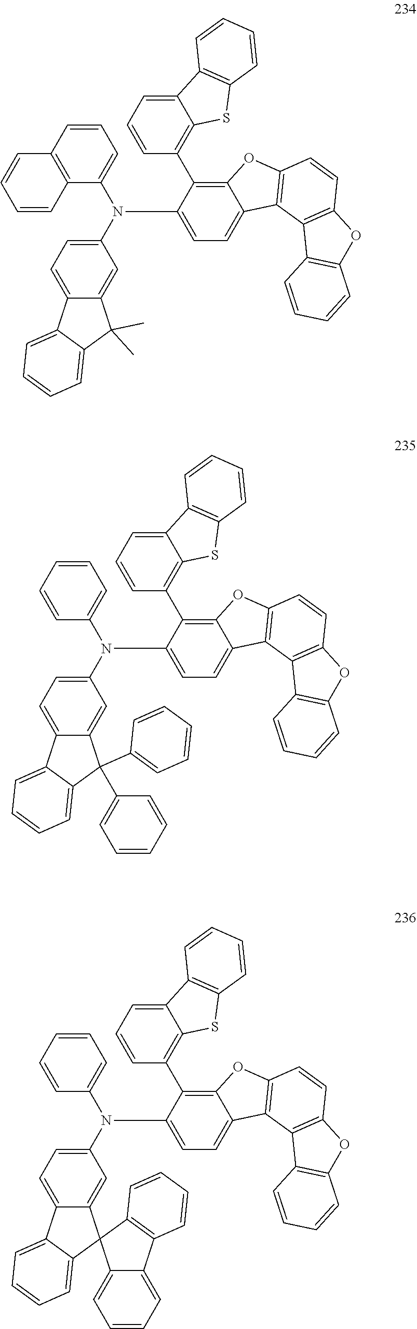 [SMILES: CC1(C)c2ccccc2-c2ccc(N(c3ccc4c(oc5ccc6oc7ccccc7c6c54)c3-c3cccc4c3sc3ccccc34)c3cccc4ccccc34)cc21 ; c1ccc(N(c2ccc3c(c2)C(c2ccccc2)(c2ccccc2)c2ccccc2-3)c2ccc3c(oc4ccc5oc6ccccc6c5c43)c2-c2cccc3c2sc2ccccc23)cc1 ; c1ccc(N(c2ccc3c(c2)C2(c4ccccc4-c4ccccc42)c2ccccc2-3)c2ccc3c(oc4ccc5oc6ccccc6c5c43)c2-c2cccc3c2sc2ccccc23)cc1]

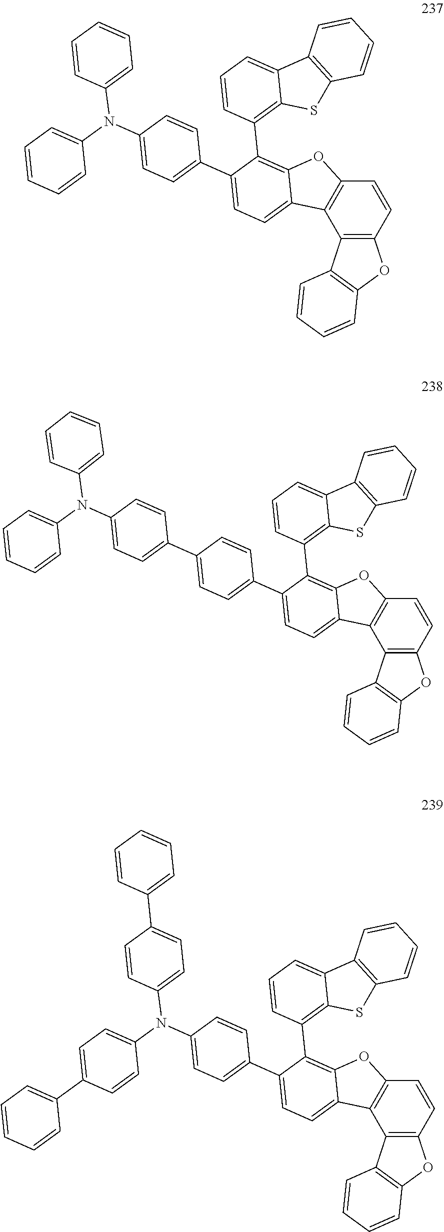 [SMILES: c1ccc(N(c2ccccc2)c2ccc(-c3ccc4c(oc5ccc6oc7ccccc7c6c54)c3-c3cccc4c3sc3ccccc34)cc2)cc1 ; c1ccc(N(c2ccccc2)c2ccc(-c3ccc(-c4ccc5c(oc6ccc7oc8ccccc8c7c65)c4-c4cccc5c4sc4ccccc45)cc3)cc2)cc1 ; c1ccc(-c2ccc(N(c3ccc(-c4ccccc4)cc3)c3ccc(-c4ccc5c(oc6ccc7oc8ccccc8c7c65)c4-c4cccc5c4sc4ccccc45)cc3)cc2)cc1]

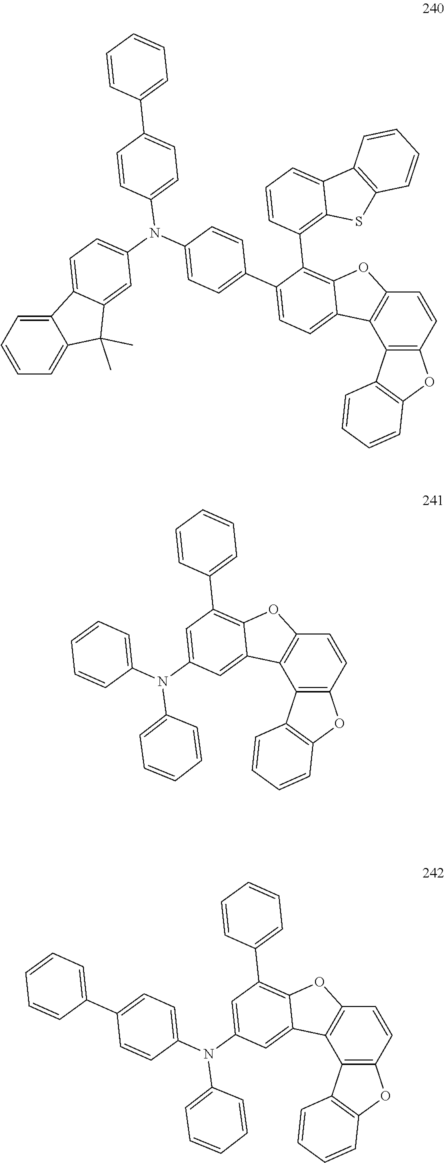 [SMILES: CC1(C)c2ccccc2-c2ccc(N(c3ccc(-c4ccccc4)cc3)c3ccc(-c4ccc5c(oc6ccc7oc8ccccc8c7c65)c4-c4cccc5c4sc4ccccc45)cc3)cc21 ; c1ccc(-c2cc(N(c3ccccc3)c3ccccc3)cc3c2oc2ccc4oc5ccccc5c4c23)cc1 ; c1ccc(-c2ccc(N(c3ccccc3)c3cc(-c4ccccc4)c4oc5ccc6oc7ccccc7c6c5c4c3)cc2)cc1]

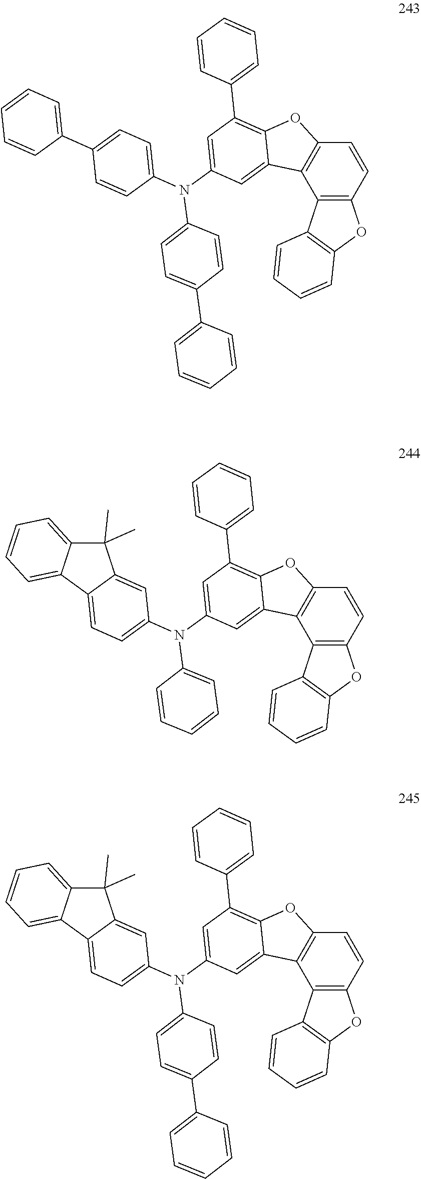 [SMILES: c1ccc(-c2ccc(N(c3ccc(-c4ccccc4)cc3)c3cc(-c4ccccc4)c4oc5ccc6oc7ccccc7c6c5c4c3)cc2)cc1 ; CC1(C)c2ccccc2-c2ccc(N(c3ccccc3)c3cc(-c4ccccc4)c4oc5ccc6oc7ccccc7c6c5c4c3)cc21 ; CC1(C)c2ccccc2-c2ccc(N(c3ccc(-c4ccccc4)cc3)c3cc(-c4ccccc4)c4oc5ccc6oc7ccccc7c6c5c4c3)cc21]

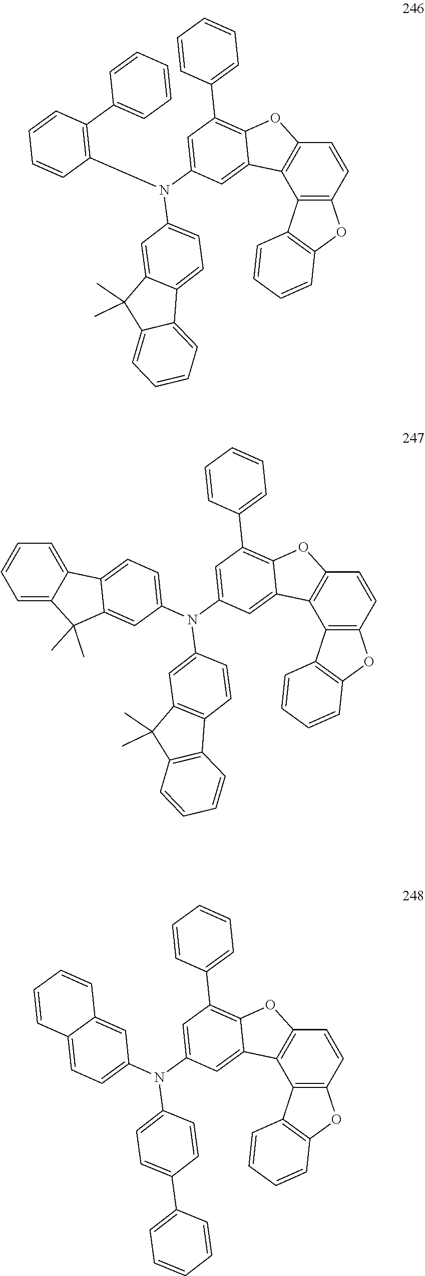 [SMILES: CC1(C)c2ccccc2-c2ccc(N(c3cc(-c4ccccc4)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4ccccc4)c4oc5ccc6oc7ccccc7c6c5c4c3)cc21 ; c1ccc(-c2ccc(N(c3ccc4ccccc4c3)c3cc(-c4ccccc4)c4oc5ccc6oc7ccccc7c6c5c4c3)cc2)cc1]

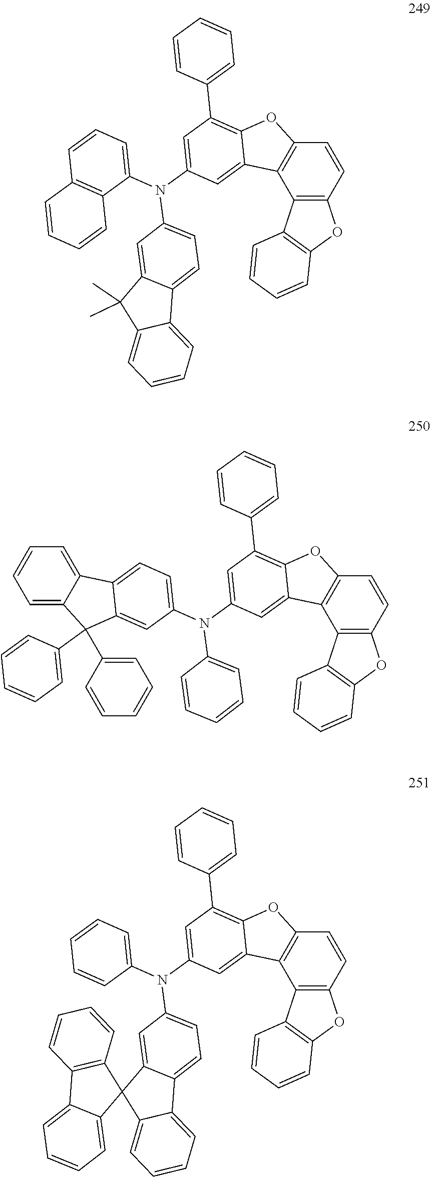 [SMILES: CC1(C)c2ccccc2-c2ccc(N(c3cc(-c4ccccc4)c4oc5ccc6oc7ccccc7c6c5c4c3)c3cccc4ccccc34)cc21 ; c1ccc(-c2cc(N(c3ccccc3)c3ccc4c(c3)C(c3ccccc3)(c3ccccc3)c3ccccc3-4)cc3c2oc2ccc4oc5ccccc5c4c23)cc1 ; c1ccc(-c2cc(N(c3ccccc3)c3ccc4c(c3)C3(c5ccccc5-c5ccccc53)c3ccccc3-4)cc3c2oc2ccc4oc5ccccc5c4c23)cc1]

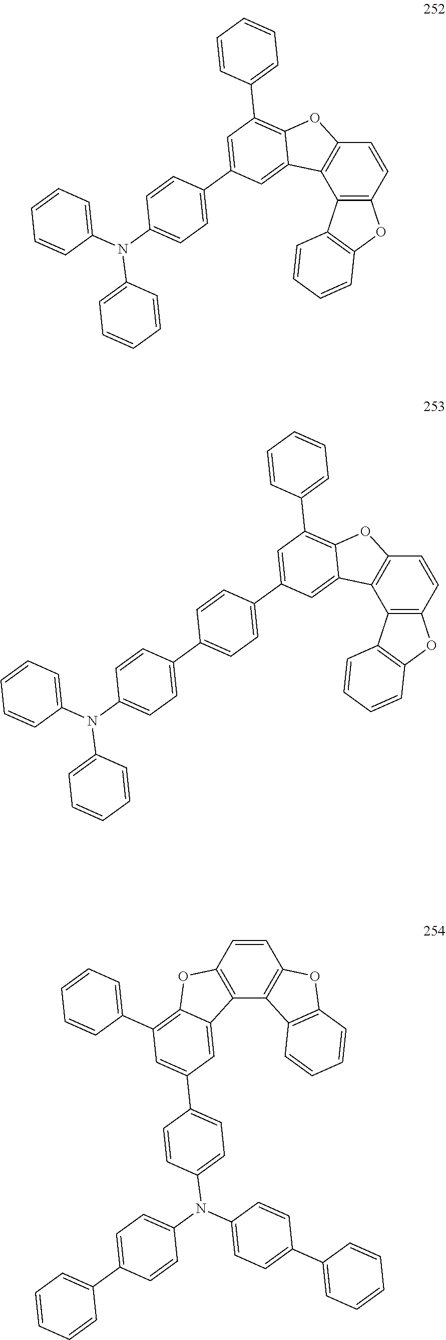 [SMILES: c1ccc(-c2cc(-c3ccc(N(c4ccccc4)c4ccccc4)cc3)cc3c2oc2ccc4oc5ccccc5c4c23)cc1 ; c1ccc(-c2cc(-c3ccc(-c4ccc(N(c5ccccc5)c5ccccc5)cc4)cc3)cc3c2oc2ccc4oc5ccccc5c4c23)cc1 ; c1ccc(-c2ccc(N(c3ccc(-c4ccccc4)cc3)c3ccc(-c4cc(-c5ccccc5)c5oc6ccc7oc8ccccc8c7c6c5c4)cc3)cc2)cc1]

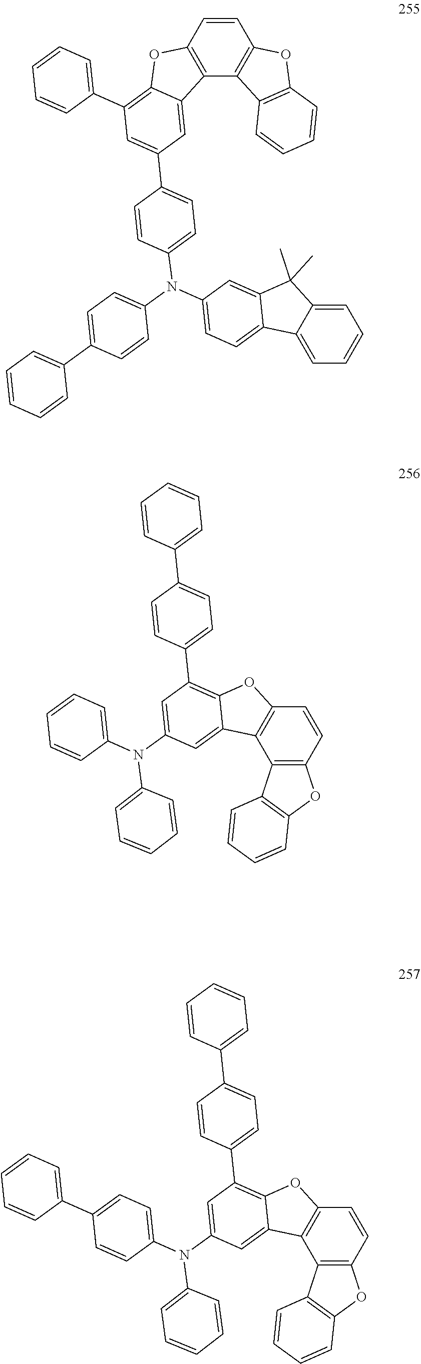 [SMILES: CC1(C)c2ccccc2-c2ccc(N(c3ccc(-c4ccccc4)cc3)c3ccc(-c4cc(-c5ccccc5)c5oc6ccc7oc8ccccc8c7c6c5c4)cc3)cc21 ; c1ccc(-c2ccc(-c3cc(N(c4ccccc4)c4ccccc4)cc4c3oc3ccc5oc6ccccc6c5c34)cc2)cc1 ; c1ccc(-c2ccc(-c3cc(N(c4ccccc4)c4ccc(-c5ccccc5)cc4)cc4c3oc3ccc5oc6ccccc6c5c34)cc2)cc1]

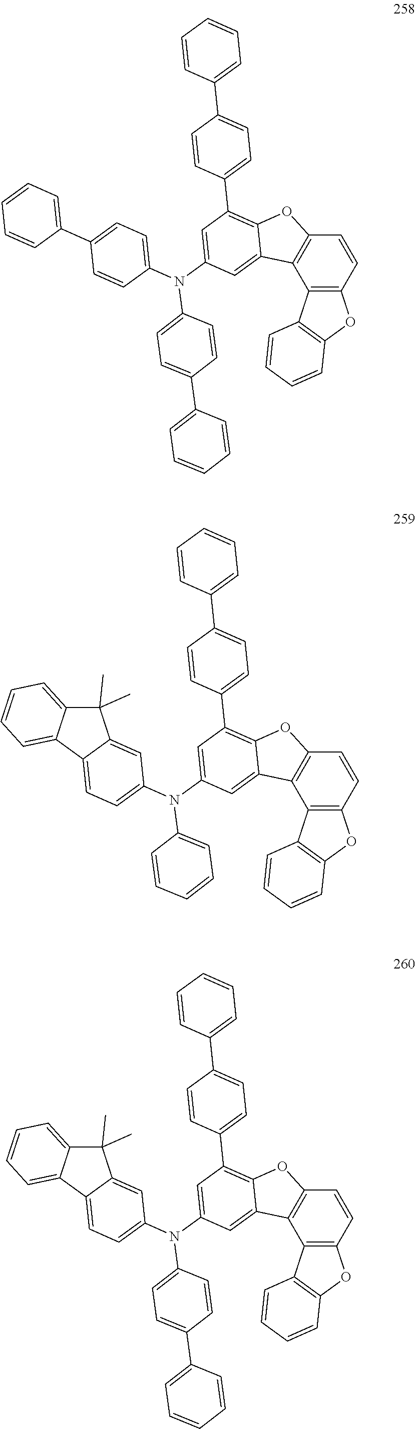 [SMILES: c1ccc(-c2ccc(-c3cc(N(c4ccc(-c5ccccc5)cc4)c4ccc(-c5ccccc5)cc4)cc4c3oc3ccc5oc6ccccc6c5c34)cc2)cc1 ; CC1(C)c2ccccc2-c2ccc(N(c3ccccc3)c3cc(-c4ccc(-c5ccccc5)cc4)c4oc5ccc6oc7ccccc7c6c5c4c3)cc21 ; CC1(C)c2ccccc2-c2ccc(N(c3ccc(-c4ccccc4)cc3)c3cc(-c4ccc(-c5ccccc5)cc4)c4oc5ccc6oc7ccccc7c6c5c4c3)cc21]

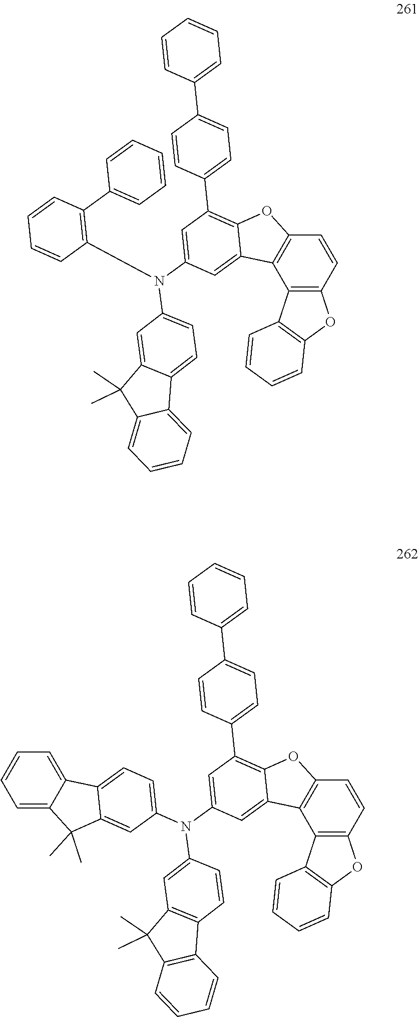 [SMILES: CC1(C)c2ccccc2-c2ccc(N(c3cc(-c4ccc(-c5ccccc5)cc4)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4ccc(-c5ccccc5)cc4)c4oc5ccc6oc7ccccc7c6c5c4c3)cc21]

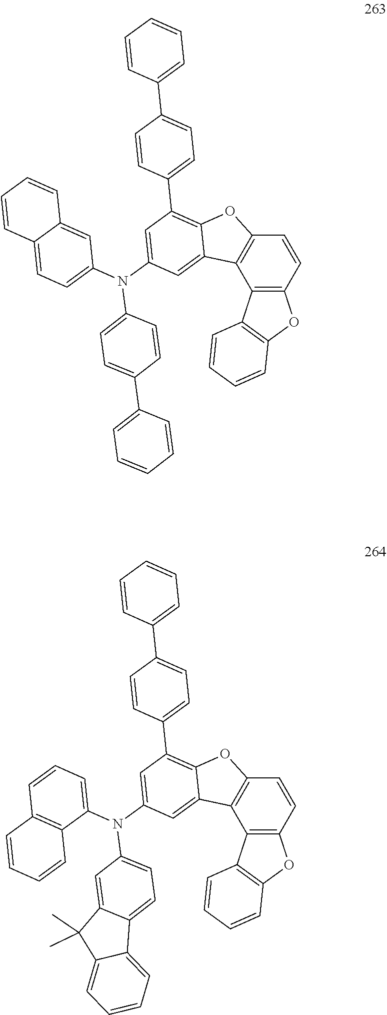 [SMILES: c1ccc(-c2ccc(-c3cc(N(c4ccc(-c5ccccc5)cc4)c4ccc5ccccc5c4)cc4c3oc3ccc5oc6ccccc6c5c34)cc2)cc1 ; CC1(C)c2ccccc2-c2ccc(N(c3cc(-c4ccc(-c5ccccc5)cc4)c4oc5ccc6oc7ccccc7c6c5c4c3)c3cccc4ccccc34)cc21]

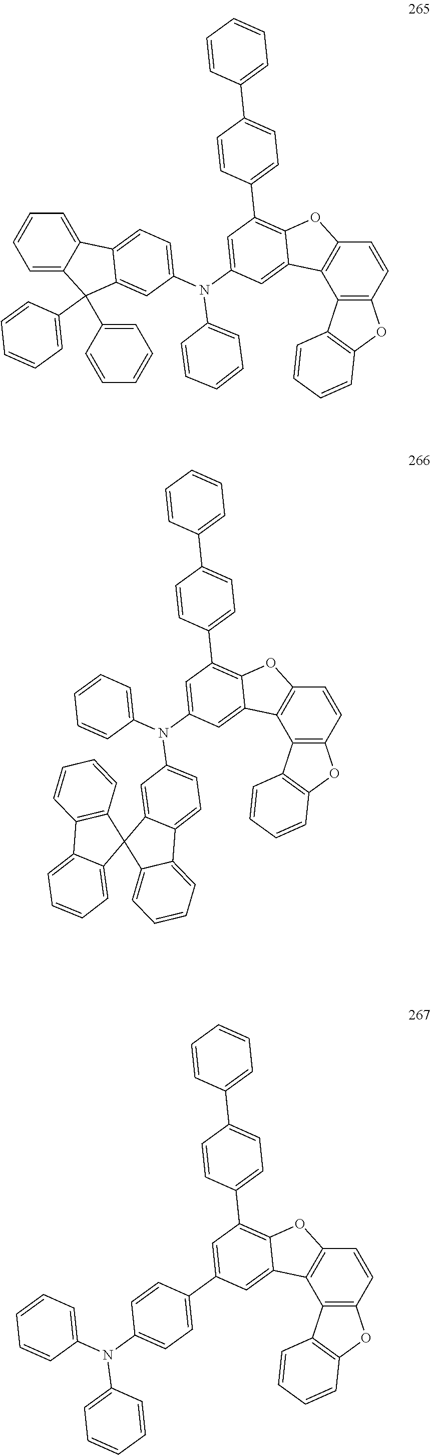 [SMILES: c1ccc(-c2ccc(-c3cc(N(c4ccccc4)c4ccc5c(c4)C(c4ccccc4)(c4ccccc4)c4ccccc4-5)cc4c3oc3ccc5oc6ccccc6c5c34)cc2)cc1 ; c1ccc(-c2ccc(-c3cc(N(c4ccccc4)c4ccc5c(c4)C4(c6ccccc6-c6ccccc64)c4ccccc4-5)cc4c3oc3ccc5oc6ccccc6c5c34)cc2)cc1 ; c1ccc(-c2ccc(-c3cc(-c4ccc(N(c5ccccc5)c5ccccc5)cc4)cc4c3oc3ccc5oc6ccccc6c5c34)cc2)cc1]

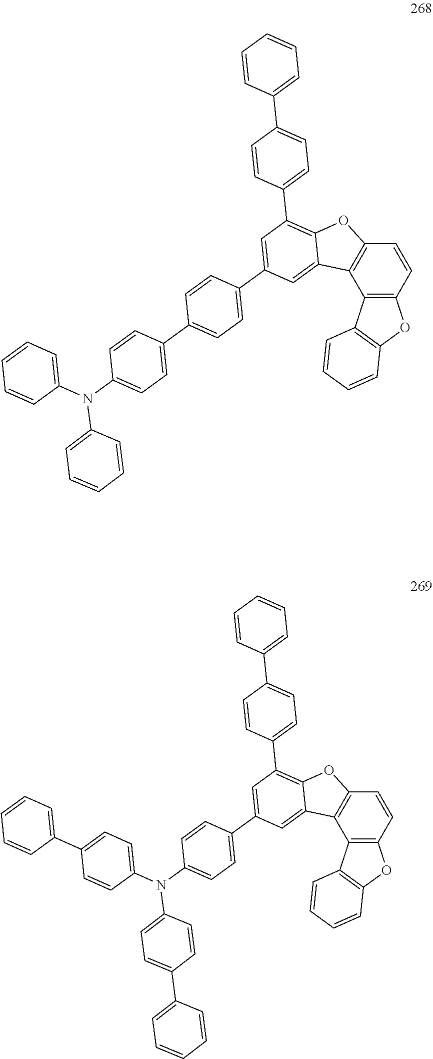 [SMILES: c1ccc(-c2ccc(-c3cc(-c4ccc(-c5ccc(N(c6ccccc6)c6ccccc6)cc5)cc4)cc4c3oc3ccc5oc6ccccc6c5c34)cc2)cc1 ; c1ccc(-c2ccc(-c3cc(-c4ccc(N(c5ccc(-c6ccccc6)cc5)c5ccc(-c6ccccc6)cc5)cc4)cc4c3oc3ccc5oc6ccccc6c5c34)cc2)cc1]

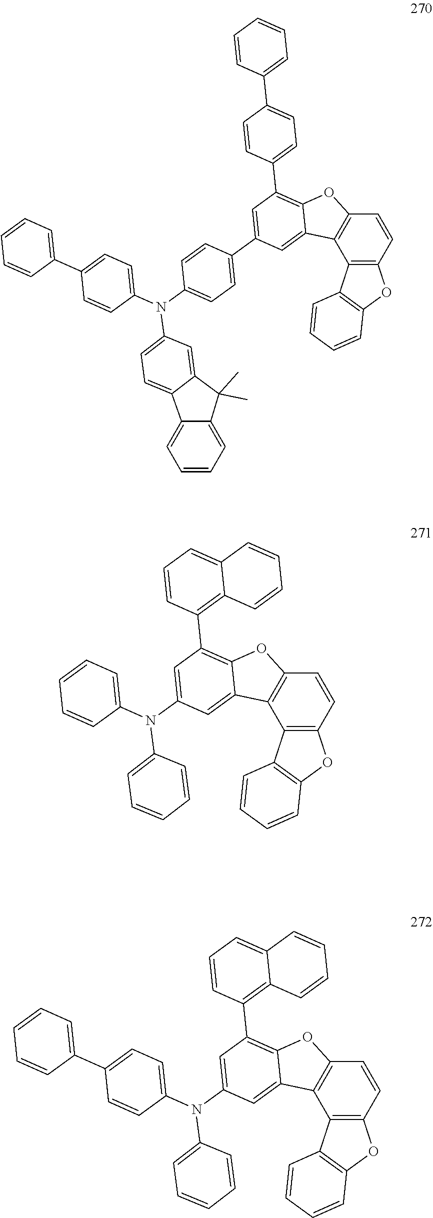 [SMILES: CC1(C)c2ccccc2-c2ccc(N(c3ccc(-c4ccccc4)cc3)c3ccc(-c4cc(-c5ccc(-c6ccccc6)cc5)c5oc6ccc7oc8ccccc8c7c6c5c4)cc3)cc21 ; c1ccc(N(c2ccccc2)c2cc(-c3cccc4ccccc34)c3oc4ccc5oc6ccccc6c5c4c3c2)cc1 ; c1ccc(-c2ccc(N(c3ccccc3)c3cc(-c4cccc5ccccc45)c4oc5ccc6oc7ccccc7c6c5c4c3)cc2)cc1]

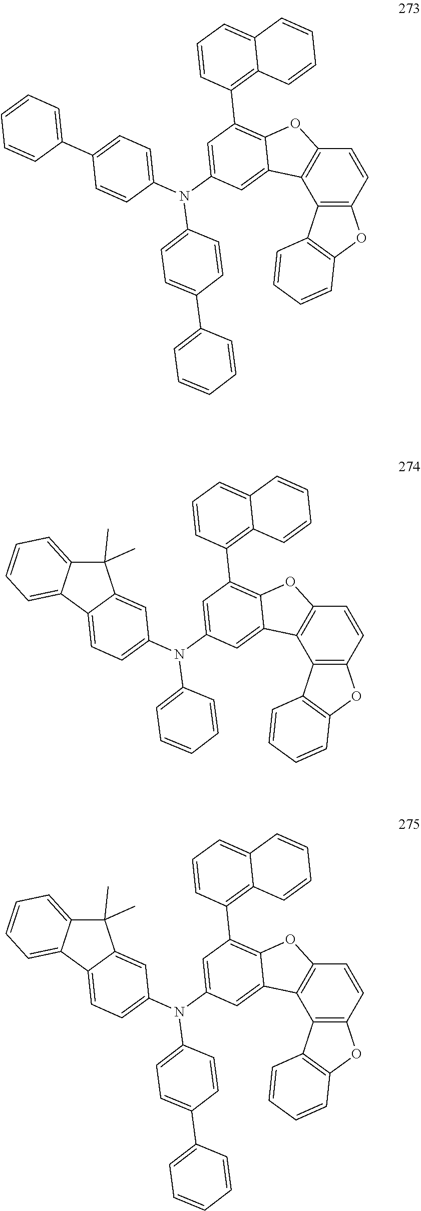 [SMILES: c1ccc(-c2ccc(N(c3ccc(-c4ccccc4)cc3)c3cc(-c4cccc5ccccc45)c4oc5ccc6oc7ccccc7c6c5c4c3)cc2)cc1 ; CC1(C)c2ccccc2-c2ccc(N(c3ccccc3)c3cc(-c4cccc5ccccc45)c4oc5ccc6oc7ccccc7c6c5c4c3)cc21 ; CC1(C)c2ccccc2-c2ccc(N(c3ccc(-c4ccccc4)cc3)c3cc(-c4cccc5ccccc45)c4oc5ccc6oc7ccccc7c6c5c4c3)cc21]

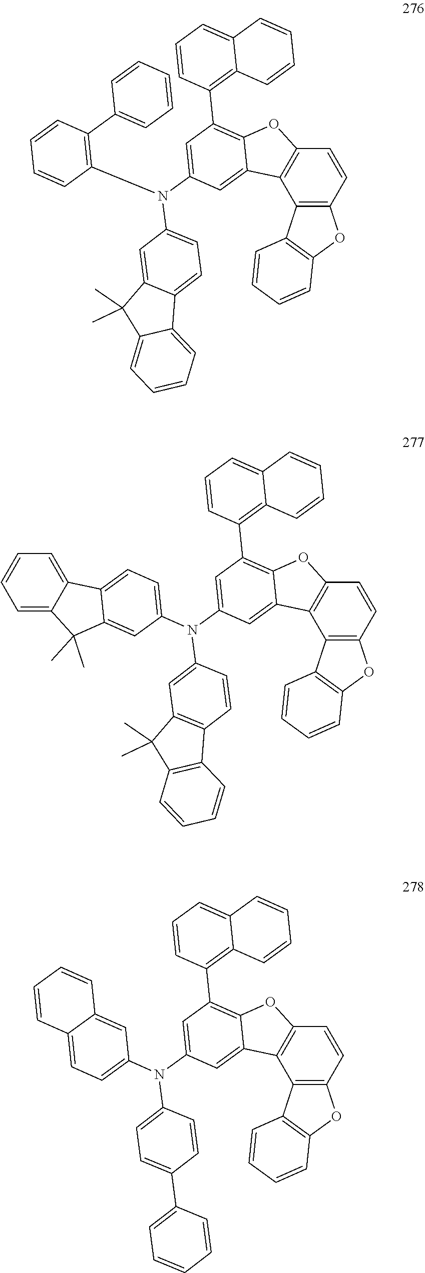 [SMILES: CC1(C)c2ccccc2-c2ccc(N(c3cc(-c4cccc5ccccc45)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4cccc5ccccc45)c4oc5ccc6oc7ccccc7c6c5c4c3)cc21 ; c1ccc(-c2ccc(N(c3ccc4ccccc4c3)c3cc(-c4cccc5ccccc45)c4oc5ccc6oc7ccccc7c6c5c4c3)cc2)cc1]

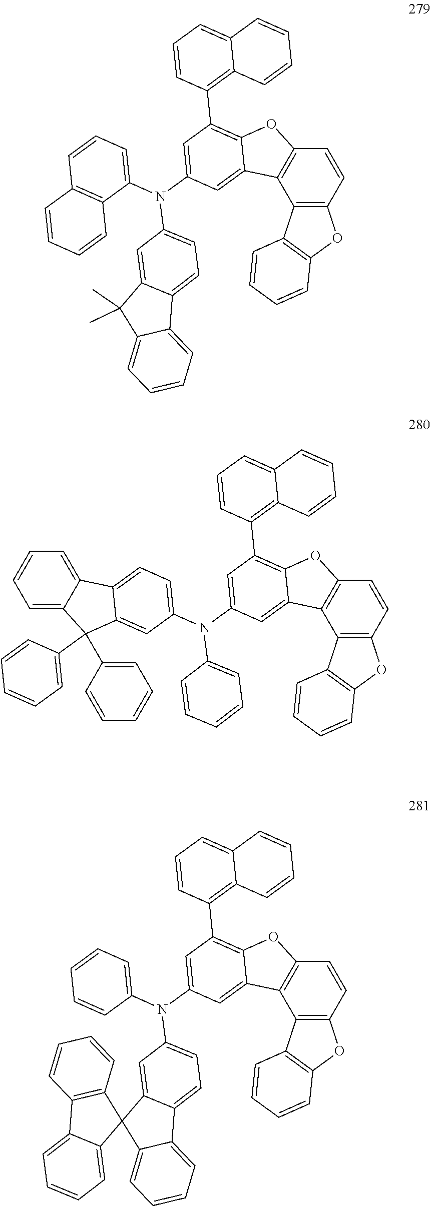 [SMILES: CC1(C)c2ccccc2-c2ccc(N(c3cc(-c4cccc5ccccc45)c4oc5ccc6oc7ccccc7c6c5c4c3)c3cccc4ccccc34)cc21 ; c1ccc(N(c2ccc3c(c2)C(c2ccccc2)(c2ccccc2)c2ccccc2-3)c2cc(-c3cccc4ccccc34)c3oc4ccc5oc6ccccc6c5c4c3c2)cc1 ; c1ccc(N(c2ccc3c(c2)C2(c4ccccc4-c4ccccc42)c2ccccc2-3)c2cc(-c3cccc4ccccc34)c3oc4ccc5oc6ccccc6c5c4c3c2)cc1]

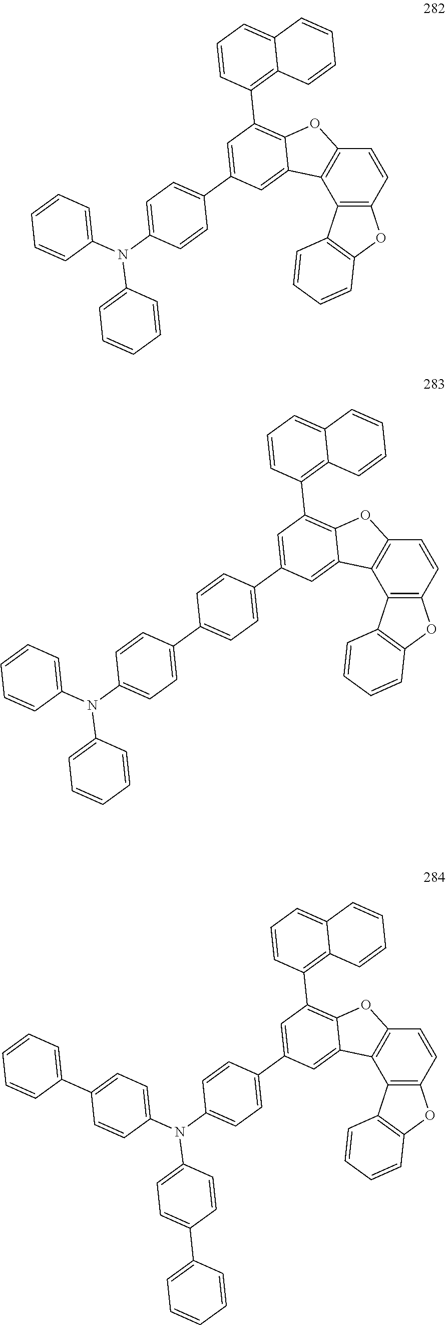 [SMILES: c1ccc(N(c2ccccc2)c2ccc(-c3cc(-c4cccc5ccccc45)c4oc5ccc6oc7ccccc7c6c5c4c3)cc2)cc1 ; c1ccc(N(c2ccccc2)c2ccc(-c3ccc(-c4cc(-c5cccc6ccccc56)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5cccc6ccccc56)c5oc6ccc7oc8ccccc8c7c6c5c4)cc3)cc2)cc1]

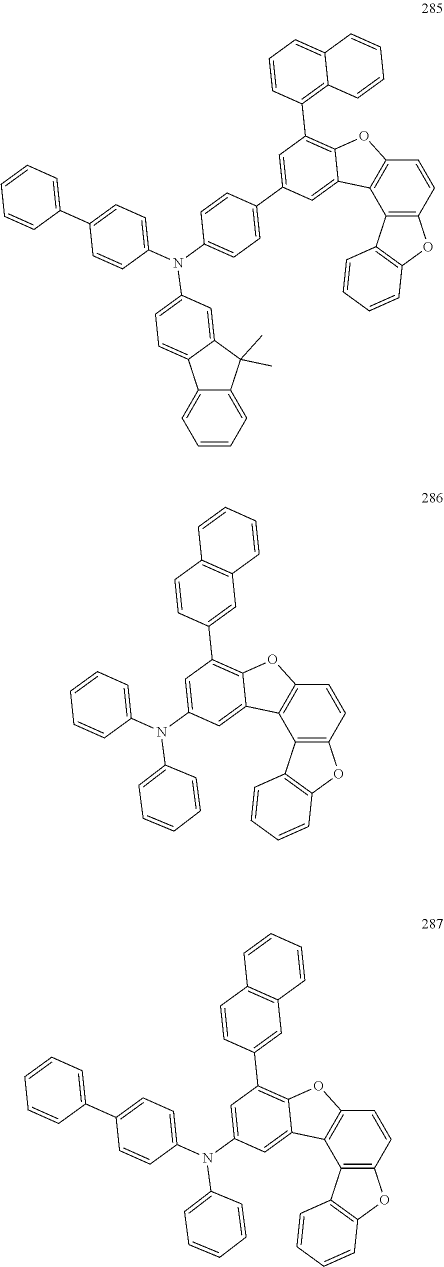 [SMILES: CC1(C)c2ccccc2-c2ccc(N(c3ccc(-c4ccccc4)cc3)c3ccc(-c4cc(-c5cccc6ccccc56)c5oc6ccc7oc8ccccc8c7c6c5c4)cc3)cc21 ; c1ccc(N(c2ccccc2)c2cc(-c3ccc4ccccc4c3)c3oc4ccc5oc6ccccc6c5c4c3c2)cc1 ; c1ccc(-c2ccc(N(c3ccccc3)c3cc(-c4ccc5ccccc5c4)c4oc5ccc6oc7ccccc7c6c5c4c3)cc2)cc1]

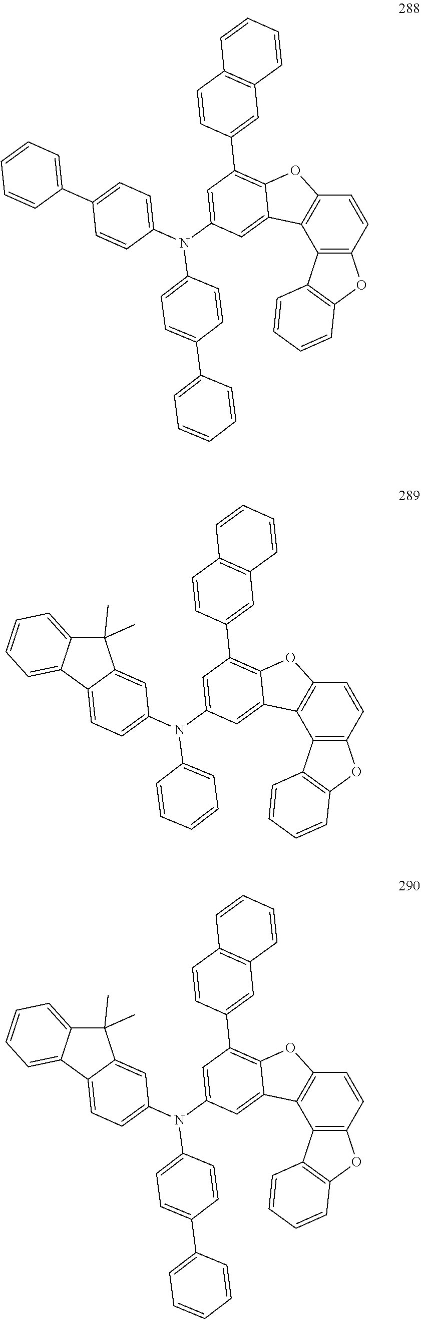 [SMILES: c1ccc(-c2ccc(N(c3ccc(-c4ccccc4)cc3)c3cc(-c4ccc5ccccc5c4)c4oc5ccc6oc7ccccc7c6c5c4c3)cc2)cc1 ; CC1(C)c2ccccc2-c2ccc(N(c3ccccc3)c3cc(-c4ccc5ccccc5c4)c4oc5ccc6oc7ccccc7c6c5c4c3)cc21 ; CC1(C)c2ccccc2-c2ccc(N(c3ccc(-c4ccccc4)cc3)c3cc(-c4ccc5ccccc5c4)c4oc5ccc6oc7ccccc7c6c5c4c3)cc21]

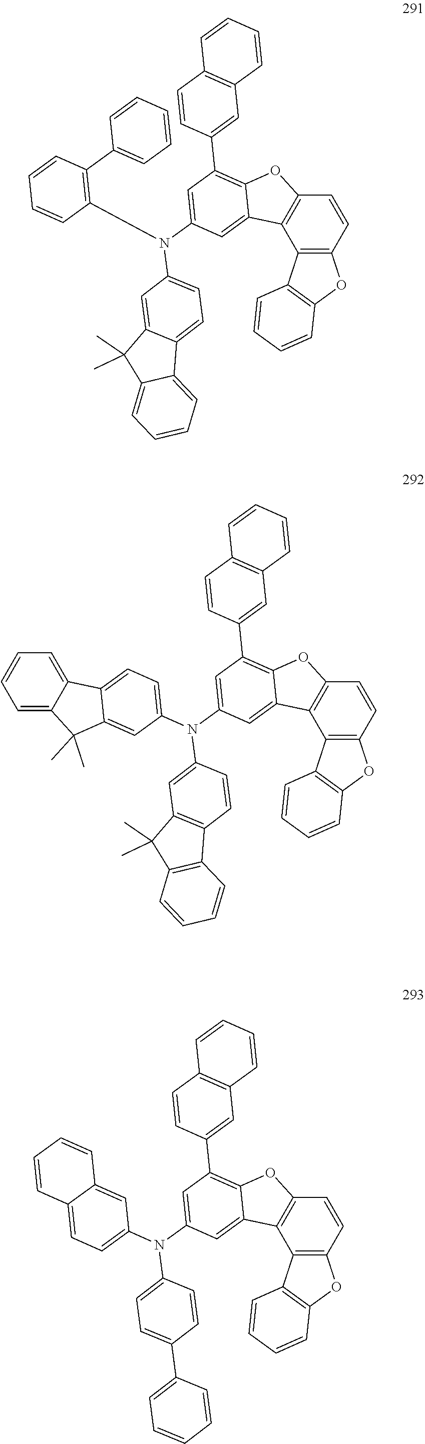 [SMILES: CC1(C)c2ccccc2-c2ccc(N(c3cc(-c4ccc5ccccc5c4)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4ccc5ccccc5c4)c4oc5ccc6oc7ccccc7c6c5c4c3)cc21 ; c1ccc(-c2ccc(N(c3ccc4ccccc4c3)c3cc(-c4ccc5ccccc5c4)c4oc5ccc6oc7ccccc7c6c5c4c3)cc2)cc1]

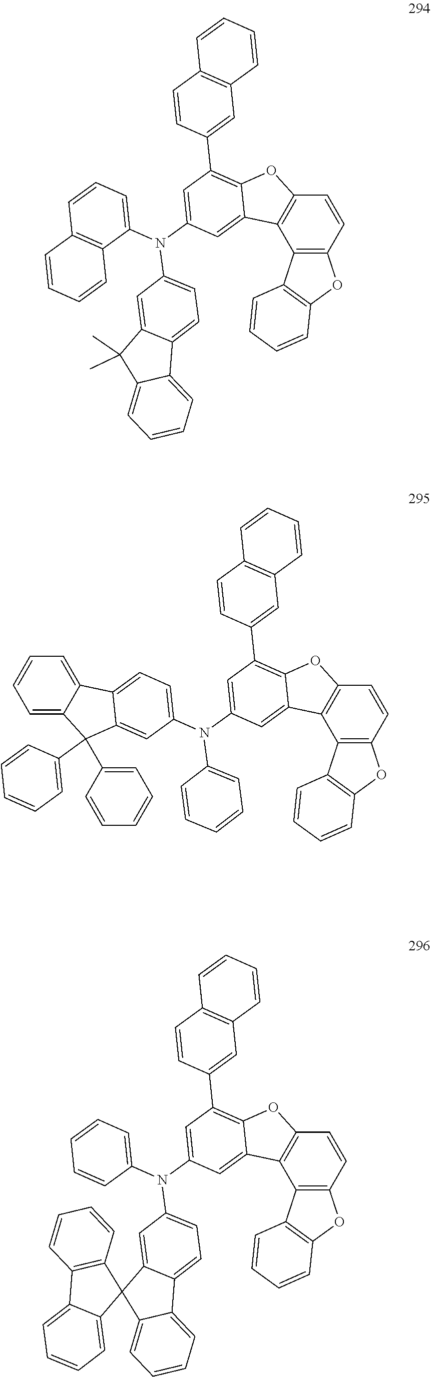 [SMILES: CC1(C)c2ccccc2-c2ccc(N(c3cc(-c4ccc5ccccc5c4)c4oc5ccc6oc7ccccc7c6c5c4c3)c3cccc4ccccc34)cc21 ; c1ccc(N(c2ccc3c(c2)C(c2ccccc2)(c2ccccc2)c2ccccc2-3)c2cc(-c3ccc4ccccc4c3)c3oc4ccc5oc6ccccc6c5c4c3c2)cc1 ; c1ccc(N(c2ccc3c(c2)C2(c4ccccc4-c4ccccc42)c2ccccc2-3)c2cc(-c3ccc4ccccc4c3)c3oc4ccc5oc6ccccc6c5c4c3c2)cc1]

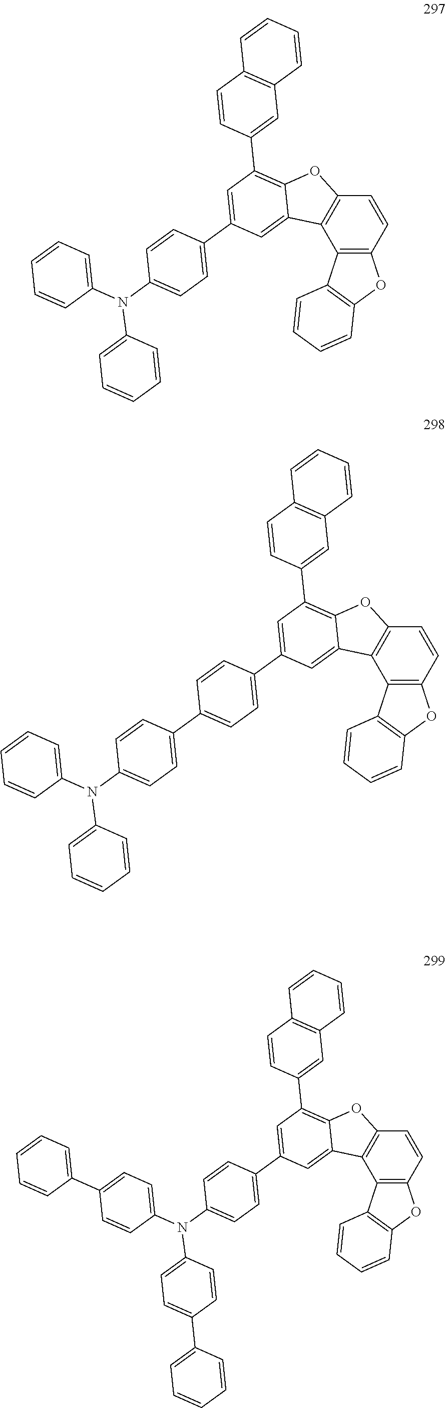 [SMILES: c1ccc(N(c2ccccc2)c2ccc(-c3cc(-c4ccc5ccccc5c4)c4oc5ccc6oc7ccccc7c6c5c4c3)cc2)cc1 ; c1ccc(N(c2ccccc2)c2ccc(-c3ccc(-c4cc(-c5ccc6ccccc6c5)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5ccc6ccccc6c5)c5oc6ccc7oc8ccccc8c7c6c5c4)cc3)cc2)cc1]

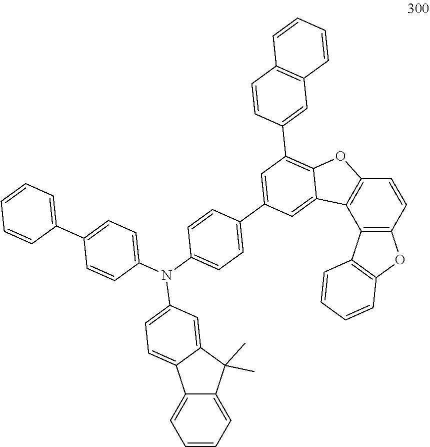 [SMILES: CC1(C)c2ccccc2-c2ccc(N(c3ccc(-c4ccccc4)cc3)c3ccc(-c4cc(-c5ccc6ccccc6c5)c5oc6ccc7oc8ccccc8c7c6c5c4)cc3)cc21]

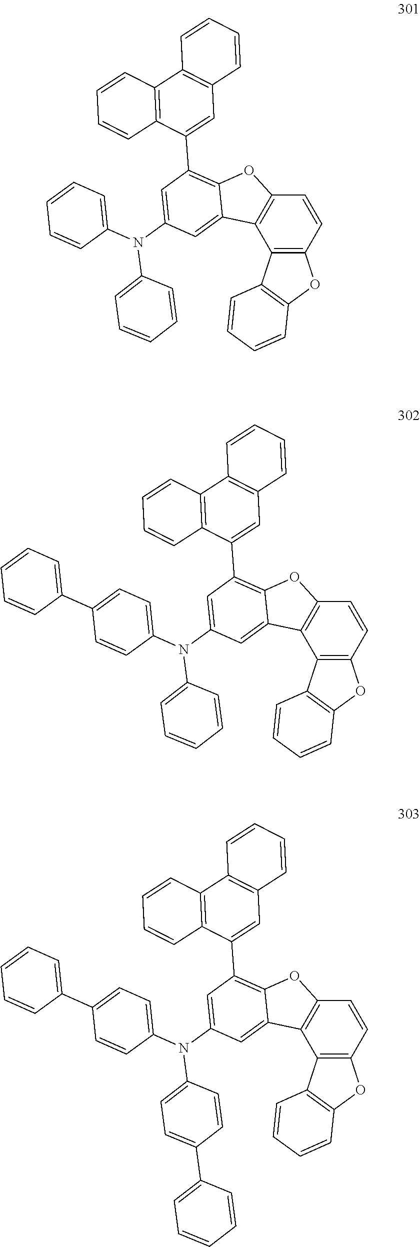 [SMILES: c1ccc(N(c2ccccc2)c2cc(-c3cc4ccccc4c4ccccc34)c3oc4ccc5oc6ccccc6c5c4c3c2)cc1 ; c1ccc(-c2ccc(N(c3ccccc3)c3cc(-c4cc5ccccc5c5ccccc45)c4oc5ccc6oc7ccccc7c6c5c4c3)cc2)cc1 ; c1ccc(-c2ccc(N(c3ccc(-c4ccccc4)cc3)c3cc(-c4cc5ccccc5c5ccccc45)c4oc5ccc6oc7ccccc7c6c5c4c3)cc2)cc1]

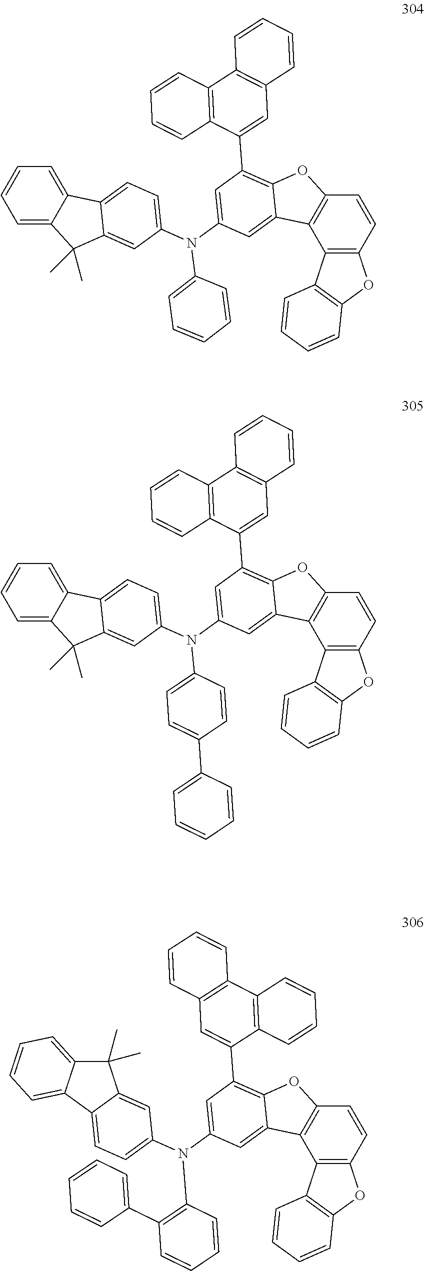 [SMILES: CC1(C)c2ccccc2-c2ccc(N(c3ccccc3)c3cc(-c4cc5ccccc5c5ccccc45)c4oc5ccc6oc7ccccc7c6c5c4c3)cc21 ; CC1(C)c2ccccc2-c2ccc(N(c3ccc(-c4ccccc4)cc3)c3cc(-c4cc5ccccc5c5ccccc45)c4oc5ccc6oc7ccccc7c6c5c4c3)cc21 ; CC1(C)c2ccccc2-c2ccc(N(c3cc(-c4cc5ccccc5c5ccccc45)c4oc5ccc6oc7ccccc7c6c5c4c3)c3ccccc3-c3ccccc3)cc21]

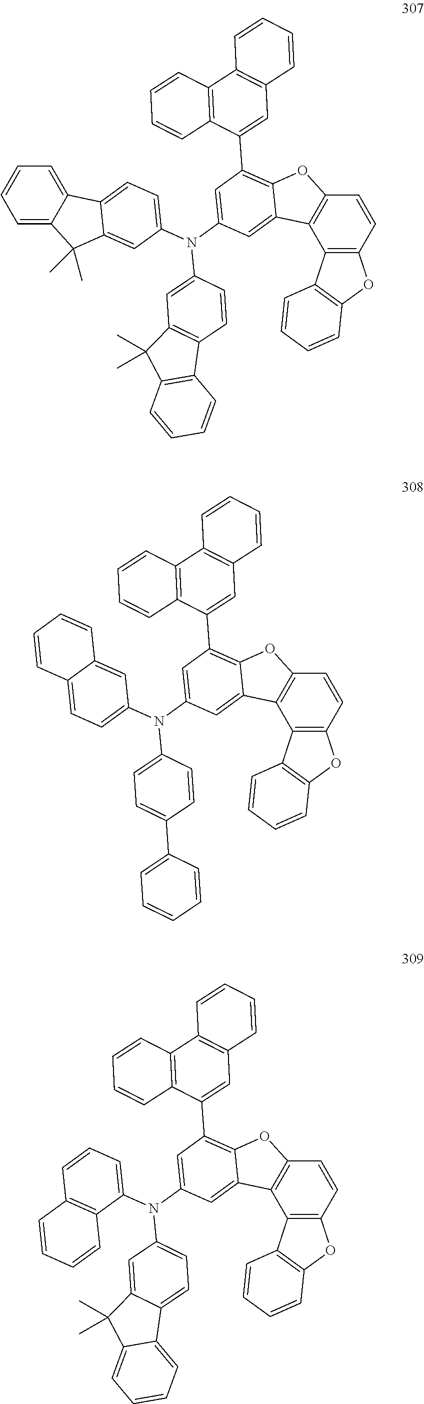 [SMILES: CC1(C)c2ccccc2-c2ccc(N(c3ccc4c(c3)C(C)(C)c3ccccc3-4)c3cc(-c4cc5ccccc5c5ccccc45)c4oc5ccc6oc7ccccc7c6c5c4c3)cc21 ; c1ccc(-c2ccc(N(c3ccc4ccccc4c3)c3cc(-c4cc5ccccc5c5ccccc45)c4oc5ccc6oc7ccccc7c6c5c4c3)cc2)cc1 ; CC1(C)c2ccccc2-c2ccc(N(c3cc(-c4cc5ccccc5c5ccccc45)c4oc5ccc6oc7ccccc7c6c5c4c3)c3cccc4ccccc34)cc21]

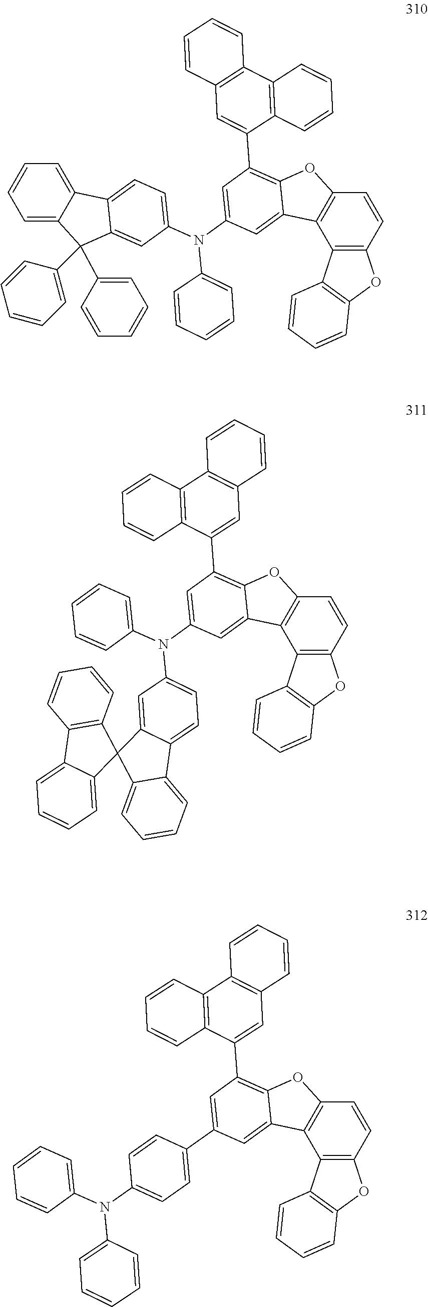 [SMILES: c1ccc(N(c2ccc3c(c2)C(c2ccccc2)(c2ccccc2)c2ccccc2-3)c2cc(-c3cc4ccccc4c4ccccc34)c3oc4ccc5oc6ccccc6c5c4c3c2)cc1 ; c1ccc(N(c2ccc3c(c2)C2(c4ccccc4-c4ccccc42)c2ccccc2-3)c2cc(-c3cc4ccccc4c4ccccc34)c3oc4ccc5oc6ccccc6c5c4c3c2)cc1 ; c1ccc(N(c2ccccc2)c2ccc(-c3cc(-c4cc5ccccc5c5ccccc45)c4oc5ccc6oc7ccccc7c6c5c4c3)cc2)cc1]

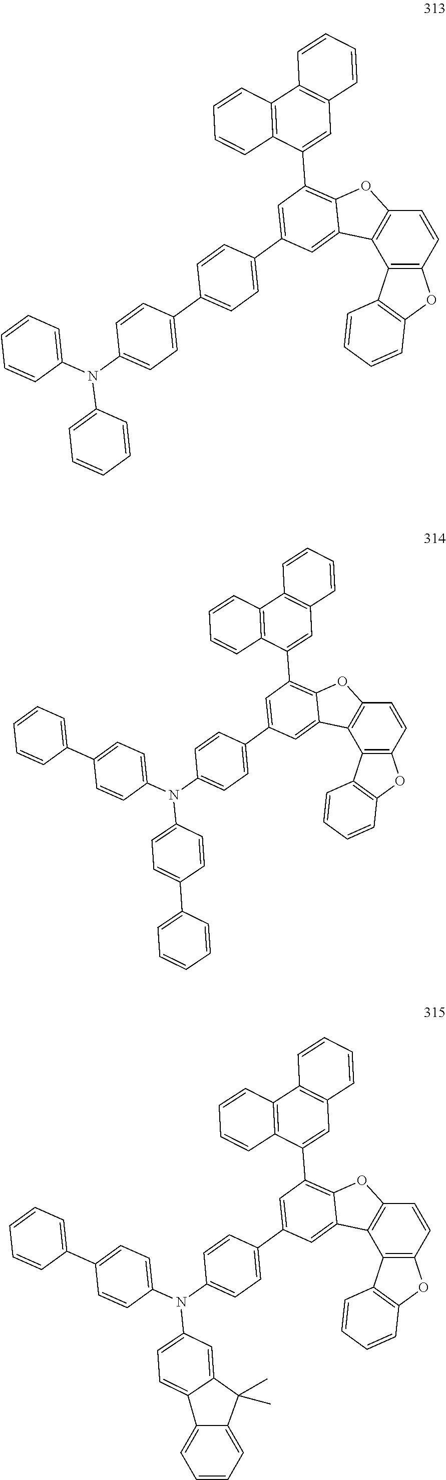 [SMILES: c1ccc(N(c2ccccc2)c2ccc(-c3ccc(-c4cc(-c5cc6ccccc6c6ccccc56)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5cc6ccccc6c6ccccc56)c5oc6ccc7oc8ccccc8c7c6c5c4)cc3)cc2)cc1 ; CC1(C)c2ccccc2-c2ccc(N(c3ccc(-c4ccccc4)cc3)c3ccc(-c4cc(-c5cc6ccccc6c6ccccc56)c5oc6ccc7oc8ccccc8c7c6c5c4)cc3)cc21]

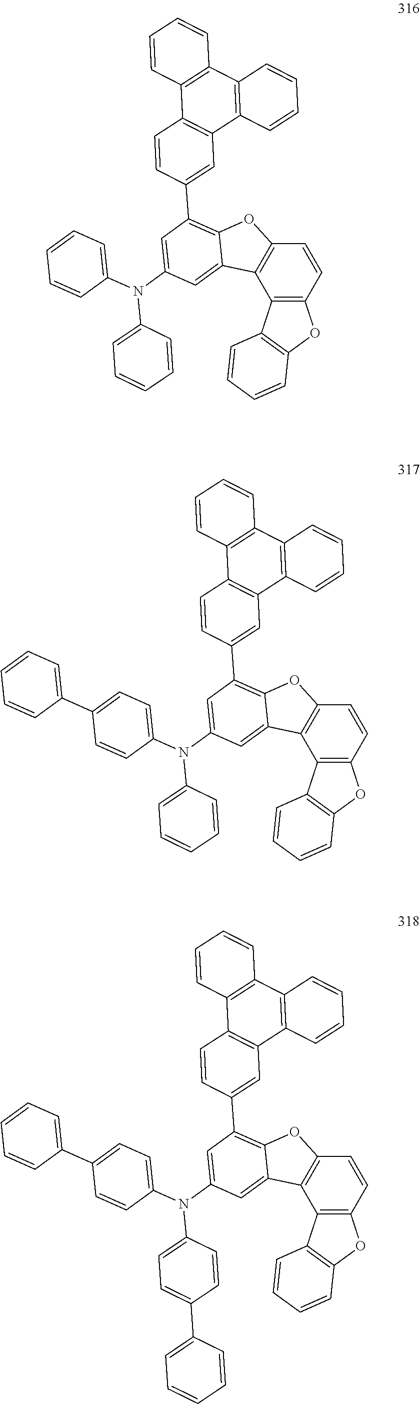 [SMILES: c1ccc(N(c2ccccc2)c2cc(-c3ccc4c5ccccc5c5ccccc5c4c3)c3oc4ccc5oc6ccccc6c5c4c3c2)cc1 ; c1ccc(-c2ccc(N(c3ccccc3)c3cc(-c4ccc5c6ccccc6c6ccccc6c5c4)c4oc5ccc6oc7ccccc7c6c5c4c3)cc2)cc1 ; c1ccc(-c2ccc(N(c3ccc(-c4ccccc4)cc3)c3cc(-c4ccc5c6ccccc6c6ccccc6c5c4)c4oc5ccc6oc7ccccc7c6c5c4c3)cc2)cc1]

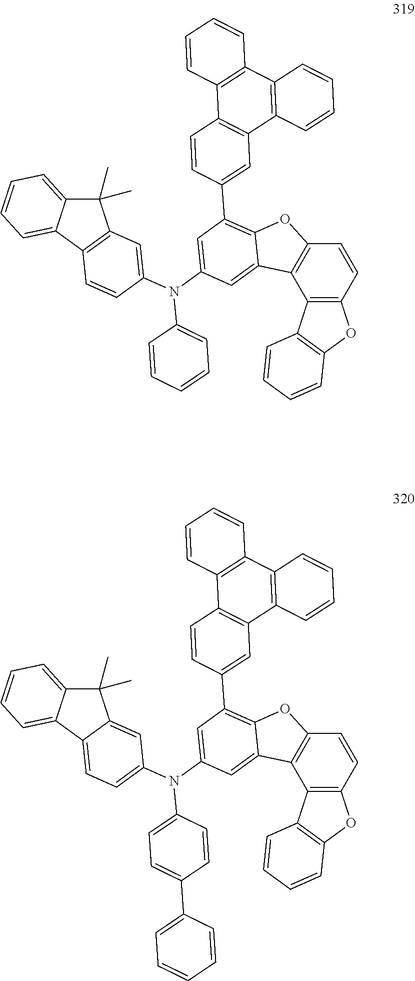 [SMILES: CC1(C)c2ccccc2-c2ccc(N(c3ccccc3)c3cc(-c4ccc5c6ccccc6c6ccccc6c5c4)c4oc5ccc6oc7ccccc7c6c5c4c3)cc21 ; CC1(C)c2ccccc2-c2ccc(N(c3ccc(-c4ccccc4)cc3)c3cc(-c4ccc5c6ccccc6c6ccccc6c5c4)c4oc5ccc6oc7ccccc7c6c5c4c3)cc21]

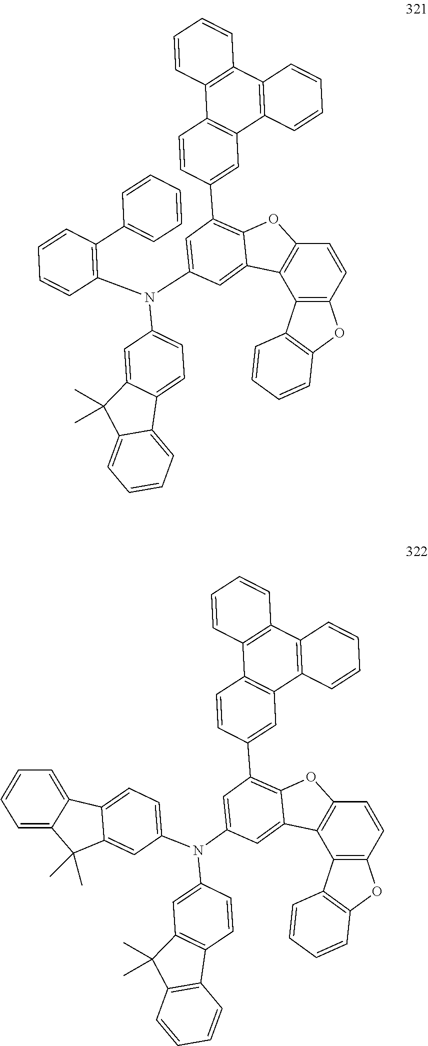 [SMILES: CC1(C)c2ccccc2-c2ccc(N(c3cc(-c4ccc5c6ccccc6c6ccccc6c5c4)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4ccc5c6ccccc6c6ccccc6c5c4)c4oc5ccc6oc7ccccc7c6c5c4c3)cc21]

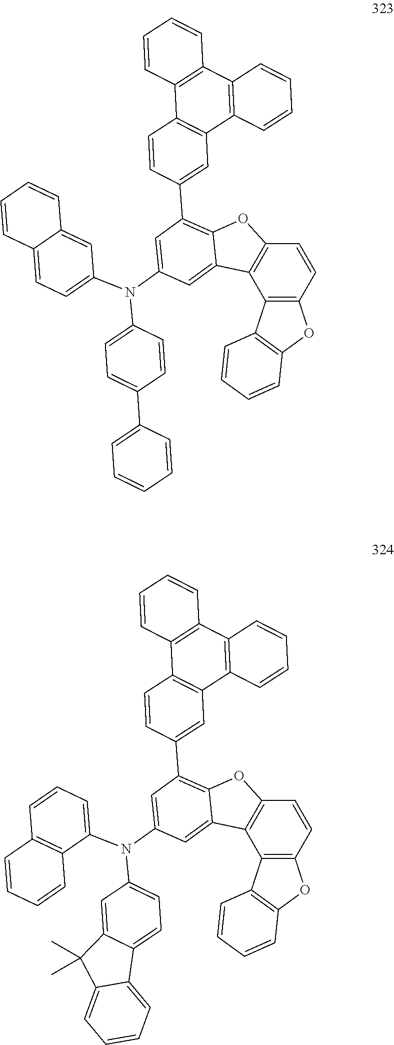 [SMILES: c1ccc(-c2ccc(N(c3ccc4ccccc4c3)c3cc(-c4ccc5c6ccccc6c6ccccc6c5c4)c4oc5ccc6oc7ccccc7c6c5c4c3)cc2)cc1 ; CC1(C)c2ccccc2-c2ccc(N(c3cc(-c4ccc5c6ccccc6c6ccccc6c5c4)c4oc5ccc6oc7ccccc7c6c5c4c3)c3cccc4ccccc34)cc21]

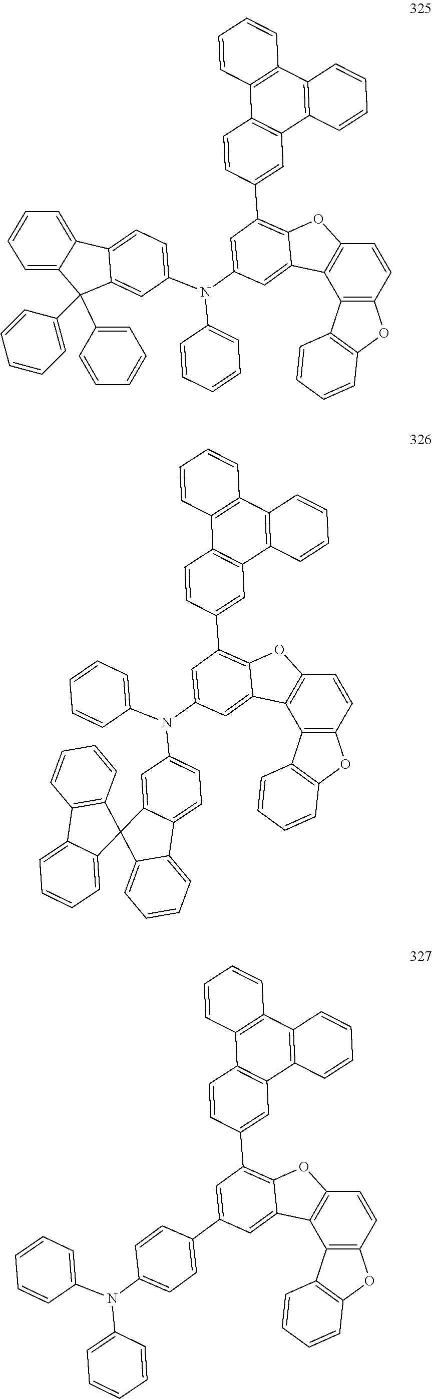 [SMILES: c1ccc(N(c2ccc3c(c2)C(c2ccccc2)(c2ccccc2)c2ccccc2-3)c2cc(-c3ccc4c5ccccc5c5ccccc5c4c3)c3oc4ccc5oc6ccccc6c5c4c3c2)cc1 ; c1ccc(N(c2ccc3c(c2)C2(c4ccccc4-c4ccccc42)c2ccccc2-3)c2cc(-c3ccc4c5ccccc5c5ccccc5c4c3)c3oc4ccc5oc6ccccc6c5c4c3c2)cc1 ; c1ccc(N(c2ccccc2)c2ccc(-c3cc(-c4ccc5c6ccccc6c6ccccc6c5c4)c4oc5ccc6oc7ccccc7c6c5c4c3)cc2)cc1]

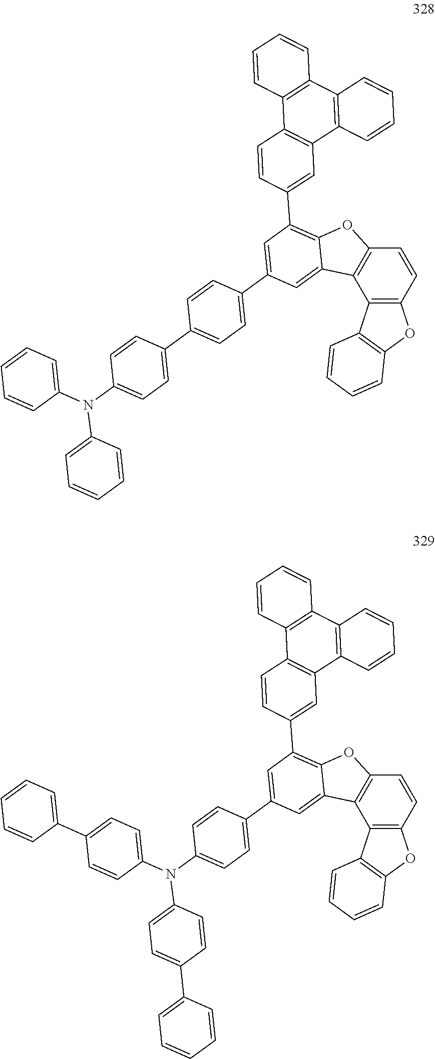 [SMILES: c1ccc(N(c2ccccc2)c2ccc(-c3ccc(-c4cc(-c5ccc6c7ccccc7c7ccccc7c6c5)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5ccc6c7ccccc7c7ccccc7c6c5)c5oc6ccc7oc8ccccc8c7c6c5c4)cc3)cc2)cc1]

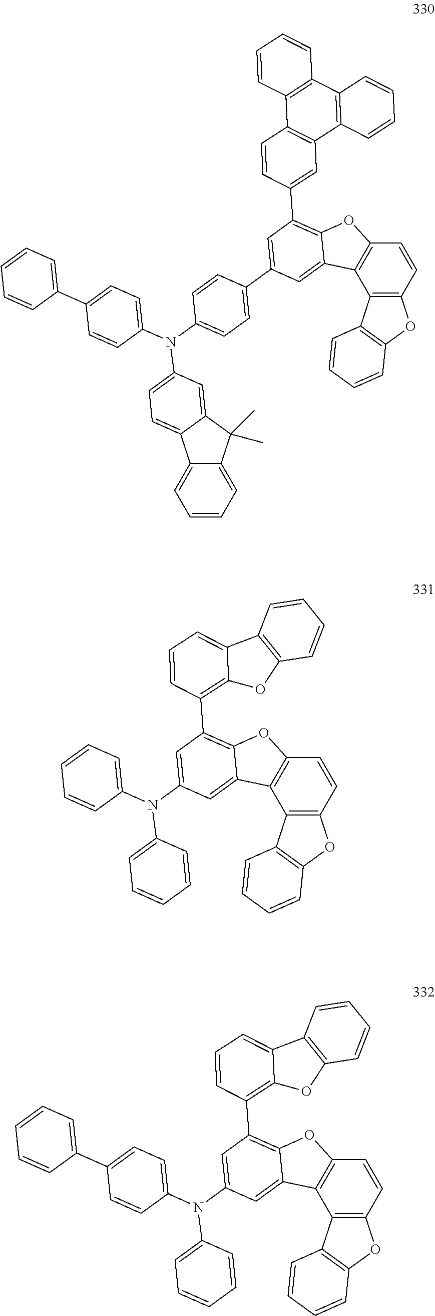 [SMILES: CC1(C)c2ccccc2-c2ccc(N(c3ccc(-c4ccccc4)cc3)c3ccc(-c4cc(-c5ccc6c7ccccc7c7ccccc7c6c5)c5oc6ccc7oc8ccccc8c7c6c5c4)cc3)cc21 ; c1ccc(N(c2ccccc2)c2cc(-c3cccc4c3oc3ccccc34)c3oc4ccc5oc6ccccc6c5c4c3c2)cc1 ; c1ccc(-c2ccc(N(c3ccccc3)c3cc(-c4cccc5c4oc4ccccc45)c4oc5ccc6oc7ccccc7c6c5c4c3)cc2)cc1]

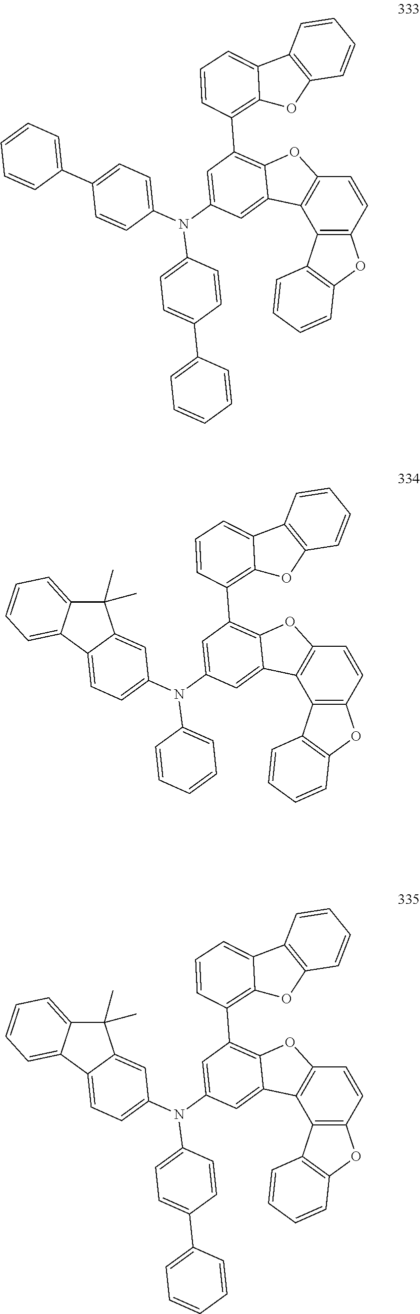 [SMILES: c1ccc(-c2ccc(N(c3ccc(-c4ccccc4)cc3)c3cc(-c4cccc5c4oc4ccccc45)c4oc5ccc6oc7ccccc7c6c5c4c3)cc2)cc1 ; CC1(C)c2ccccc2-c2ccc(N(c3ccccc3)c3cc(-c4cccc5c4oc4ccccc45)c4oc5ccc6oc7ccccc7c6c5c4c3)cc21 ; CC1(C)c2ccccc2-c2ccc(N(c3ccc(-c4ccccc4)cc3)c3cc(-c4cccc5c4oc4ccccc45)c4oc5ccc6oc7ccccc7c6c5c4c3)cc21]

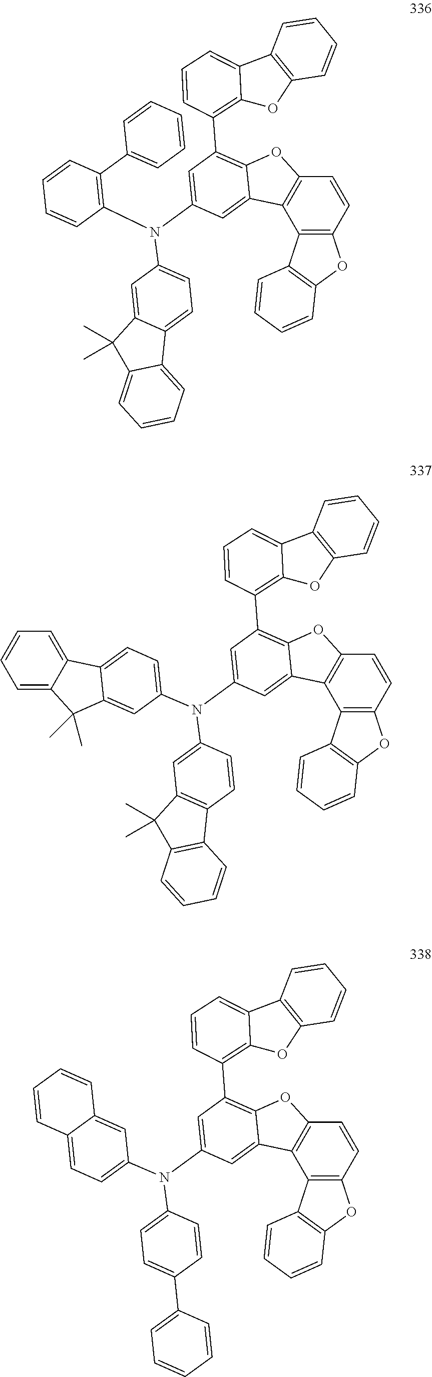 [SMILES: CC1(C)c2ccccc2-c2ccc(N(c3cc(-c4cccc5c4oc4ccccc45)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4cccc5c4oc4ccccc45)c4oc5ccc6oc7ccccc7c6c5c4c3)cc21 ; c1ccc(-c2ccc(N(c3ccc4ccccc4c3)c3cc(-c4cccc5c4oc4ccccc45)c4oc5ccc6oc7ccccc7c6c5c4c3)cc2)cc1]

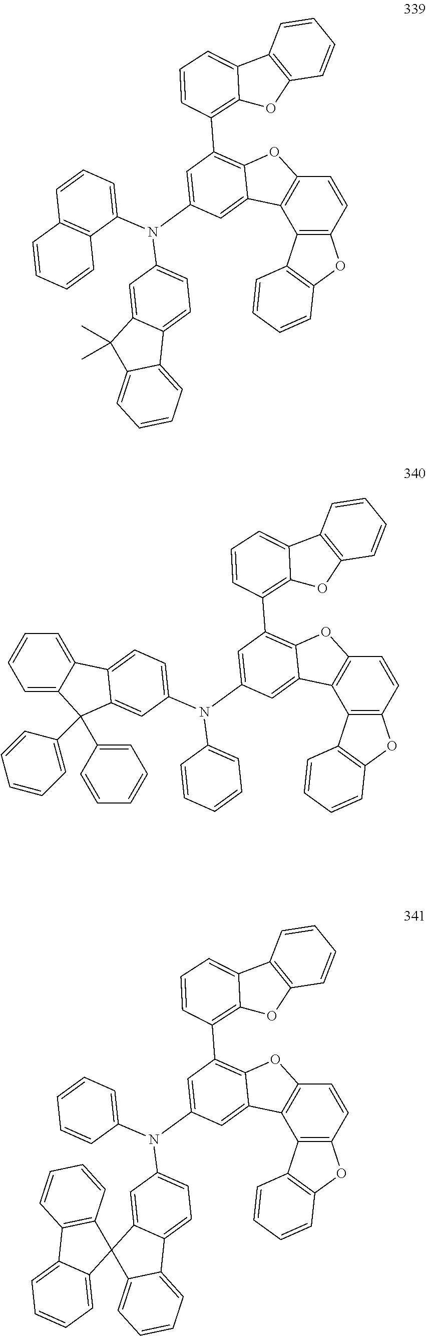 [SMILES: CC1(C)c2ccccc2-c2ccc(N(c3cc(-c4cccc5c4oc4ccccc45)c4oc5ccc6oc7ccccc7c6c5c4c3)c3cccc4ccccc34)cc21 ; c1ccc(N(c2ccc3c(c2)C(c2ccccc2)(c2ccccc2)c2ccccc2-3)c2cc(-c3cccc4c3oc3ccccc34)c3oc4ccc5oc6ccccc6c5c4c3c2)cc1 ; c1ccc(N(c2ccc3c(c2)C2(c4ccccc4-c4ccccc42)c2ccccc2-3)c2cc(-c3cccc4c3oc3ccccc34)c3oc4ccc5oc6ccccc6c5c4c3c2)cc1]

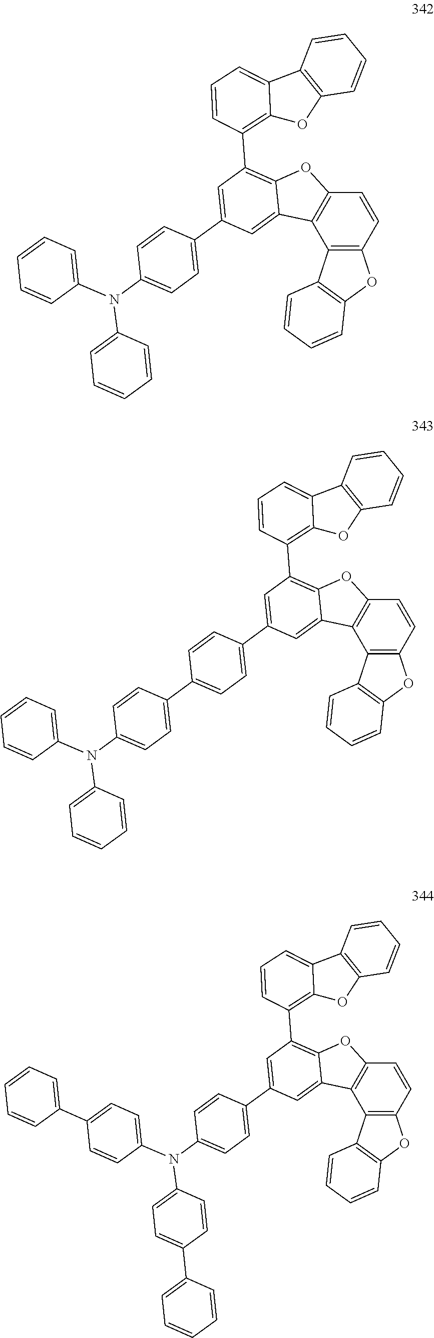 [SMILES: c1ccc(N(c2ccccc2)c2ccc(-c3cc(-c4cccc5c4oc4ccccc45)c4oc5ccc6oc7ccccc7c6c5c4c3)cc2)cc1 ; c1ccc(N(c2ccccc2)c2ccc(-c3ccc(-c4cc(-c5cccc6c5oc5ccccc56)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5cccc6c5oc5ccccc56)c5oc6ccc7oc8ccccc8c7c6c5c4)cc3)cc2)cc1]

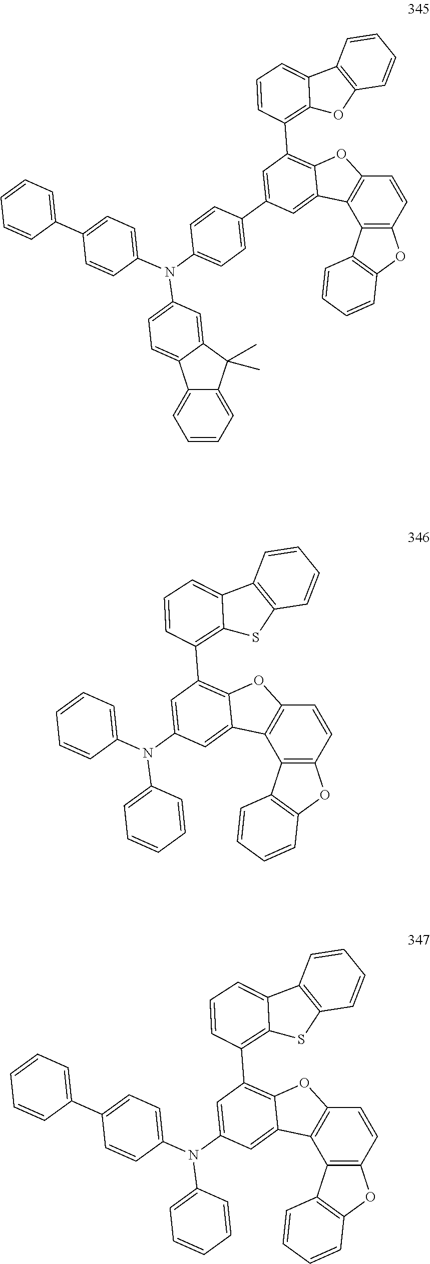 [SMILES: CC1(C)c2ccccc2-c2ccc(N(c3ccc(-c4ccccc4)cc3)c3ccc(-c4cc(-c5cccc6c5oc5ccccc56)c5oc6ccc7oc8ccccc8c7c6c5c4)cc3)cc21 ; c1ccc(N(c2ccccc2)c2cc(-c3cccc4c3sc3ccccc34)c3oc4ccc5oc6ccccc6c5c4c3c2)cc1 ; c1ccc(-c2ccc(N(c3ccccc3)c3cc(-c4cccc5c4sc4ccccc45)c4oc5ccc6oc7ccccc7c6c5c4c3)cc2)cc1]

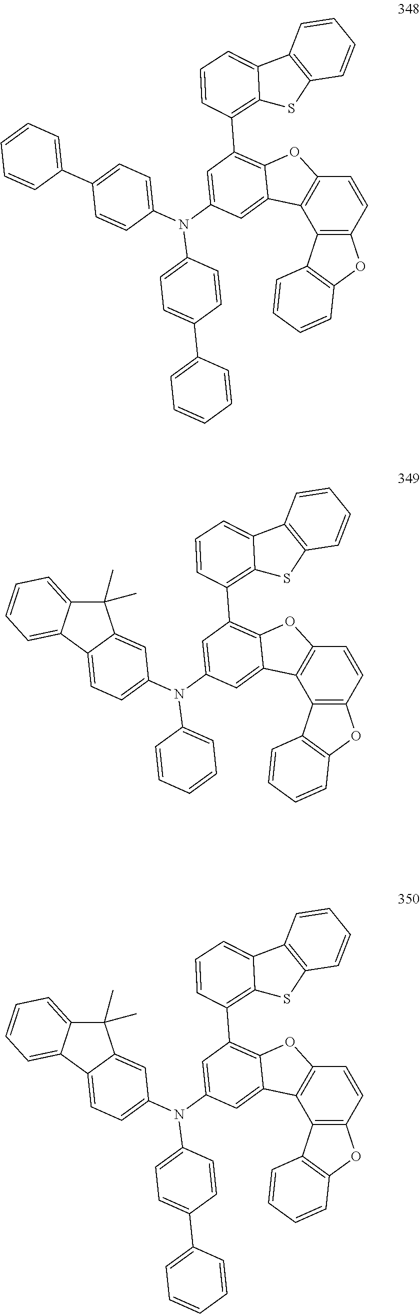 [SMILES: c1ccc(-c2ccc(N(c3ccc(-c4ccccc4)cc3)c3cc(-c4cccc5c4sc4ccccc45)c4oc5ccc6oc7ccccc7c6c5c4c3)cc2)cc1 ; CC1(C)c2ccccc2-c2ccc(N(c3ccccc3)c3cc(-c4cccc5c4sc4ccccc45)c4oc5ccc6oc7ccccc7c6c5c4c3)cc21 ; CC1(C)c2ccccc2-c2ccc(N(c3ccc(-c4ccccc4)cc3)c3cc(-c4cccc5c4sc4ccccc45)c4oc5ccc6oc7ccccc7c6c5c4c3)cc21]

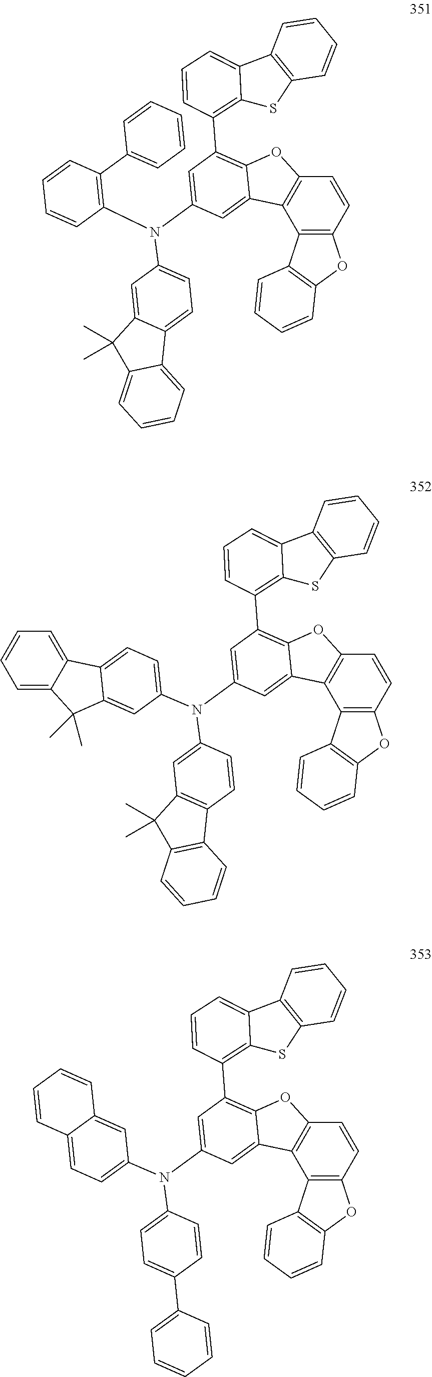 [SMILES: CC1(C)c2ccccc2-c2ccc(N(c3cc(-c4cccc5c4sc4ccccc45)c4oc5ccc6oc7ccccc7c6c5c4c3)c3ccccc3-c3ccccc3)cc21 ; CC1(C)c2ccccc2-c2ccc(N(c3ccc4c(c3)C(C)(C)c3ccccc3-4)c3cc(-c4cccc5c4sc4ccccc45)c4oc5ccc6oc7ccccc7c6c5c4c3)cc21 ; c1ccc(-c2ccc(N(c3ccc4ccccc4c3)c3cc(-c4cccc5c4sc4ccccc45)c4oc5ccc6oc7ccccc7c6c5c4c3)cc2)cc1]

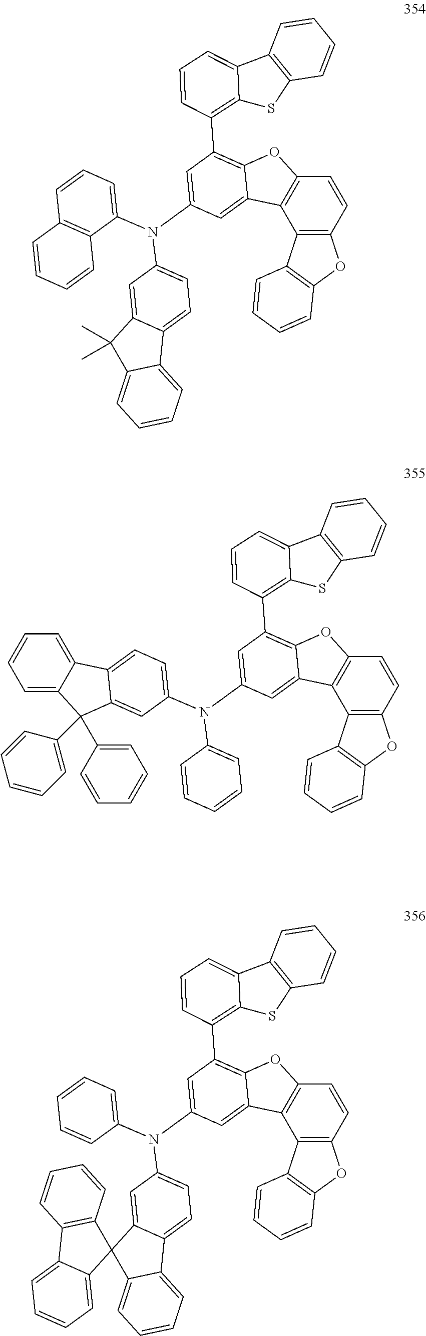 [SMILES: CC1(C)c2ccccc2-c2ccc(N(c3cc(-c4cccc5c4sc4ccccc45)c4oc5ccc6oc7ccccc7c6c5c4c3)c3cccc4ccccc34)cc21 ; c1ccc(N(c2ccc3c(c2)C(c2ccccc2)(c2ccccc2)c2ccccc2-3)c2cc(-c3cccc4c3sc3ccccc34)c3oc4ccc5oc6ccccc6c5c4c3c2)cc1 ; c1ccc(N(c2ccc3c(c2)C2(c4ccccc4-c4ccccc42)c2ccccc2-3)c2cc(-c3cccc4c3sc3ccccc34)c3oc4ccc5oc6ccccc6c5c4c3c2)cc1]

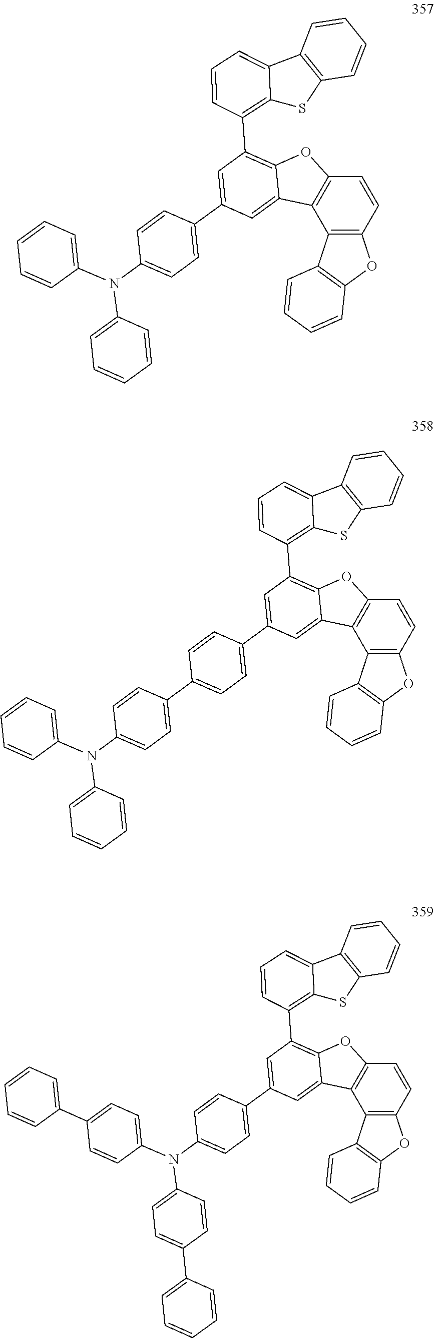 [SMILES: c1ccc(N(c2ccccc2)c2ccc(-c3cc(-c4cccc5c4sc4ccccc45)c4oc5ccc6oc7ccccc7c6c5c4c3)cc2)cc1 ; c1ccc(N(c2ccccc2)c2ccc(-c3ccc(-c4cc(-c5cccc6c5sc5ccccc56)c5oc6ccc7oc8ccccc8c7c6c5c4)cc3)cc2)cc1 ; c1ccc(-c2ccc(N(c3ccc(-c4ccccc4)cc3)c3ccc(-c4cc(-c5cccc6c5sc5ccccc56)c5oc6ccc7oc8ccccc8c7c6c5c4)cc3)cc2)cc1]

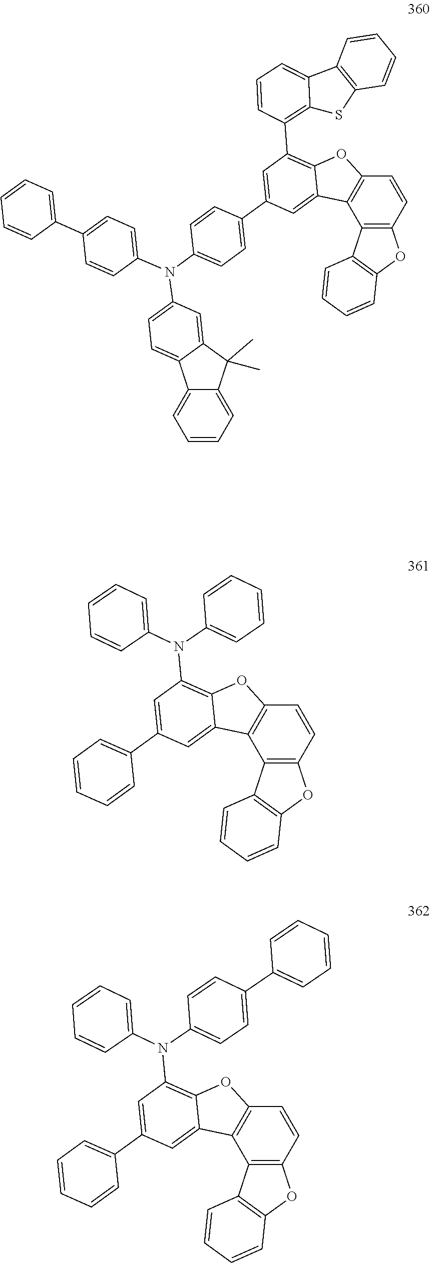 [SMILES: CC1(C)c2ccccc2-c2ccc(N(c3ccc(-c4ccccc4)cc3)c3ccc(-c4cc(-c5cccc6c5sc5ccccc56)c5oc6ccc7oc8ccccc8c7c6c5c4)cc3)cc21 ; c1ccc(-c2cc(N(c3ccccc3)c3ccccc3)c3oc4ccc5oc6ccccc6c5c4c3c2)cc1 ; c1ccc(-c2ccc(N(c3ccccc3)c3cc(-c4ccccc4)cc4c3oc3ccc5oc6ccccc6c5c34)cc2)cc1]

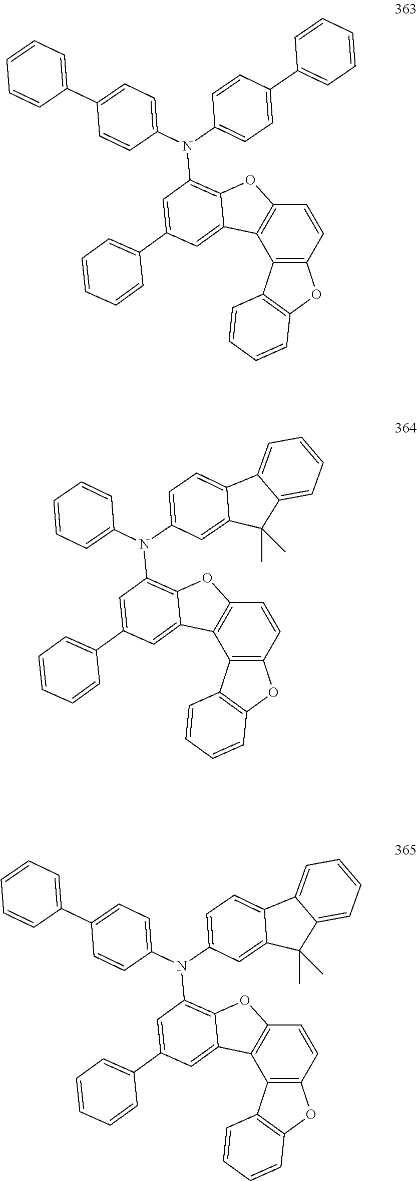 [SMILES: c1ccc(-c2ccc(N(c3ccc(-c4ccccc4)cc3)c3cc(-c4ccccc4)cc4c3oc3ccc5oc6ccccc6c5c34)cc2)cc1 ; CC1(C)c2ccccc2-c2ccc(N(c3ccccc3)c3cc(-c4ccccc4)cc4c3oc3ccc5oc6ccccc6c5c34)cc21 ; CC1(C)c2ccccc2-c2ccc(N(c3ccc(-c4ccccc4)cc3)c3cc(-c4ccccc4)cc4c3oc3ccc5oc6ccccc6c5c34)cc21]

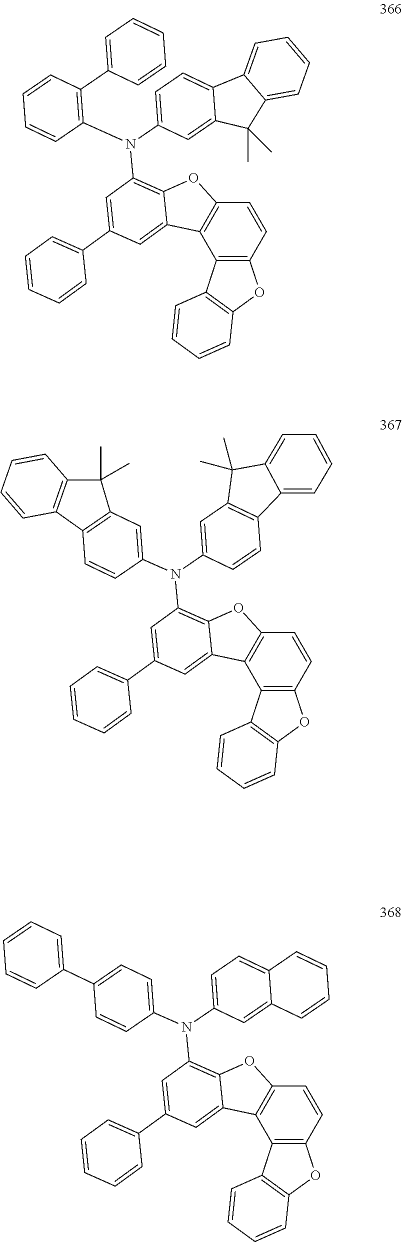 [SMILES: CC1(C)c2ccccc2-c2ccc(N(c3ccccc3-c3ccccc3)c3cc(-c4ccccc4)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4ccccc4)cc4c3oc3ccc5oc6ccccc6c5c34)cc21 ; c1ccc(-c2ccc(N(c3ccc4ccccc4c3)c3cc(-c4ccccc4)cc4c3oc3ccc5oc6ccccc6c5c34)cc2)cc1]

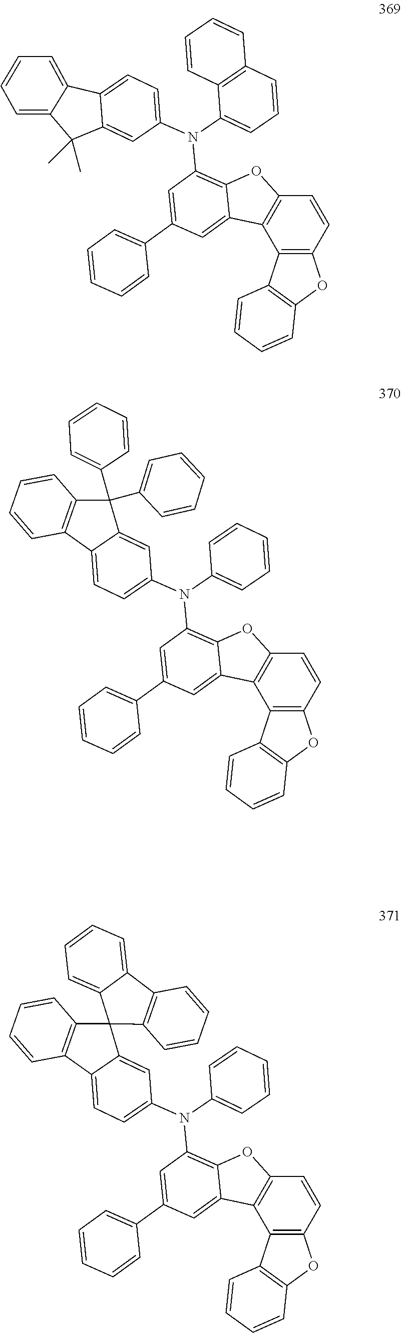 [SMILES: CC1(C)c2ccccc2-c2ccc(N(c3cccc4ccccc34)c3cc(-c4ccccc4)cc4c3oc3ccc5oc6ccccc6c5c34)cc21 ; c1ccc(-c2cc(N(c3ccccc3)c3ccc4c(c3)C(c3ccccc3)(c3ccccc3)c3ccccc3-4)c3oc4ccc5oc6ccccc6c5c4c3c2)cc1 ; c1ccc(-c2cc(N(c3ccccc3)c3ccc4c(c3)C3(c5ccccc5-c5ccccc53)c3ccccc3-4)c3oc4ccc5oc6ccccc6c5c4c3c2)cc1]

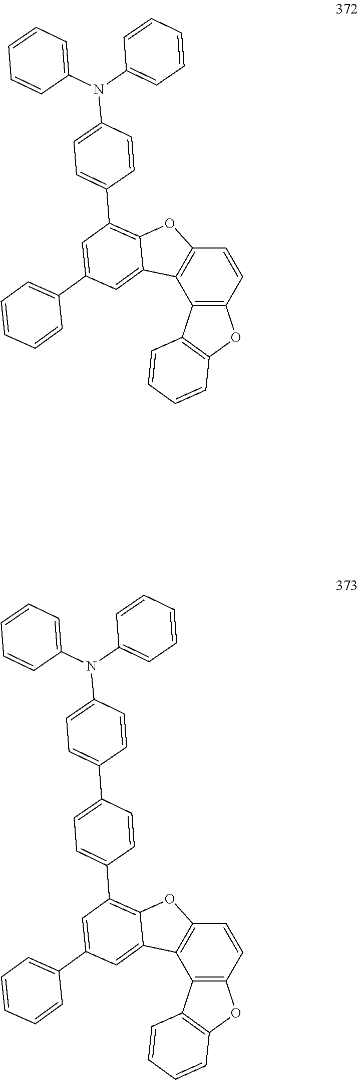 [SMILES: c1ccc(-c2cc(-c3ccc(N(c4ccccc4)c4ccccc4)cc3)c3oc4ccc5oc6ccccc6c5c4c3c2)cc1 ; c1ccc(-c2cc(-c3ccc(-c4ccc(N(c5ccccc5)c5ccccc5)cc4)cc3)c3oc4ccc5oc6ccccc6c5c4c3c2)cc1]

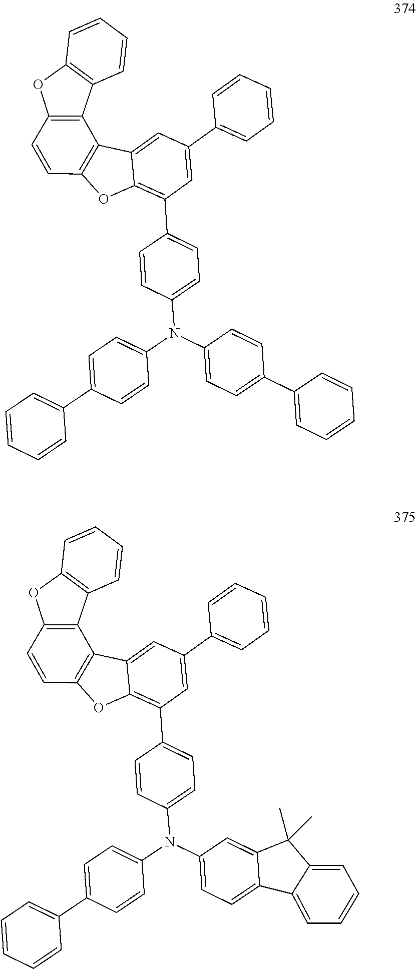 [SMILES: c1ccc(-c2ccc(N(c3ccc(-c4ccccc4)cc3)c3ccc(-c4cc(-c5ccccc5)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5ccccc5)cc5c4oc4ccc6oc7ccccc7c6c45)cc3)cc21]

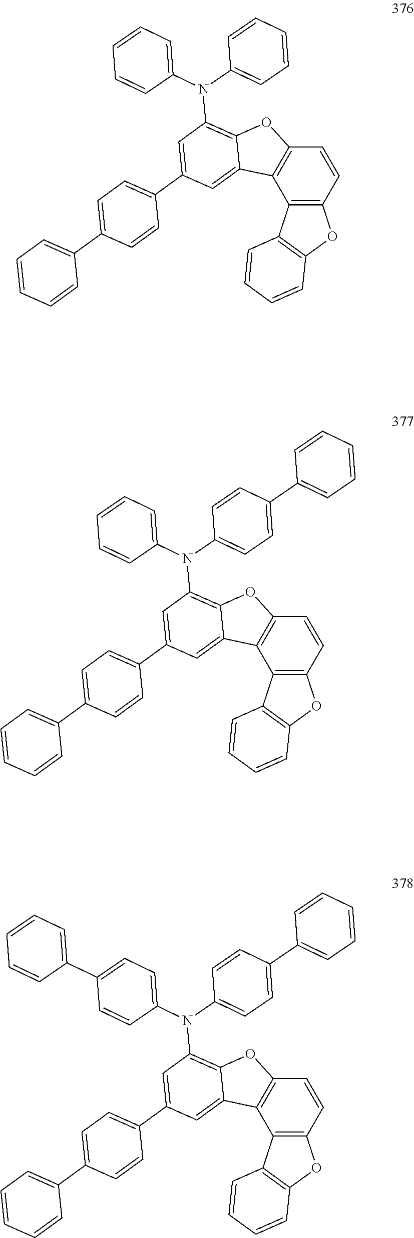 [SMILES: c1ccc(-c2ccc(-c3cc(N(c4ccccc4)c4ccccc4)c4oc5ccc6oc7ccccc7c6c5c4c3)cc2)cc1 ; c1ccc(-c2ccc(-c3cc(N(c4ccccc4)c4ccc(-c5ccccc5)cc4)c4oc5ccc6oc7ccccc7c6c5c4c3)cc2)cc1 ; c1ccc(-c2ccc(-c3cc(N(c4ccc(-c5ccccc5)cc4)c4ccc(-c5ccccc5)cc4)c4oc5ccc6oc7ccccc7c6c5c4c3)cc2)cc1]

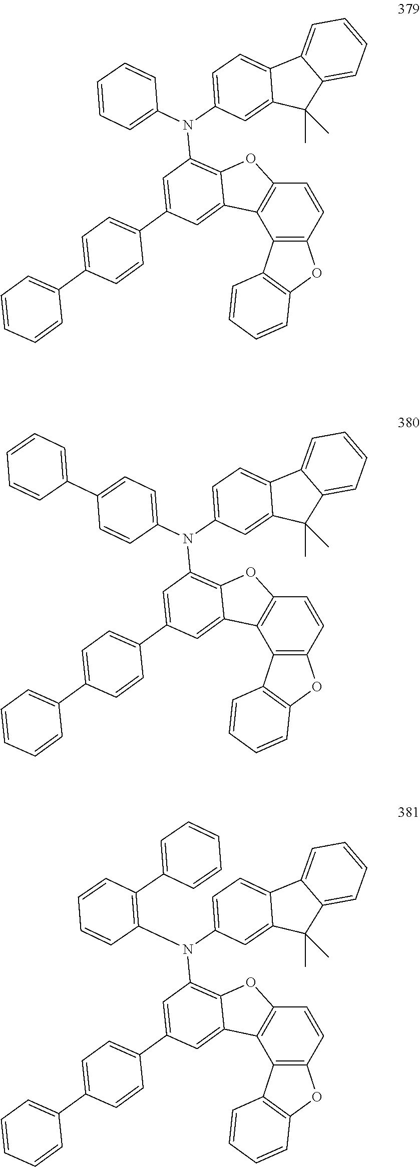 [SMILES: CC1(C)c2ccccc2-c2ccc(N(c3ccccc3)c3cc(-c4ccc(-c5ccccc5)cc4)cc4c3oc3ccc5oc6ccccc6c5c34)cc21 ; CC1(C)c2ccccc2-c2ccc(N(c3ccc(-c4ccccc4)cc3)c3cc(-c4ccc(-c5ccccc5)cc4)cc4c3oc3ccc5oc6ccccc6c5c34)cc21 ; CC1(C)c2ccccc2-c2ccc(N(c3ccccc3-c3ccccc3)c3cc(-c4ccc(-c5ccccc5)cc4)cc4c3oc3ccc5oc6ccccc6c5c34)cc21]

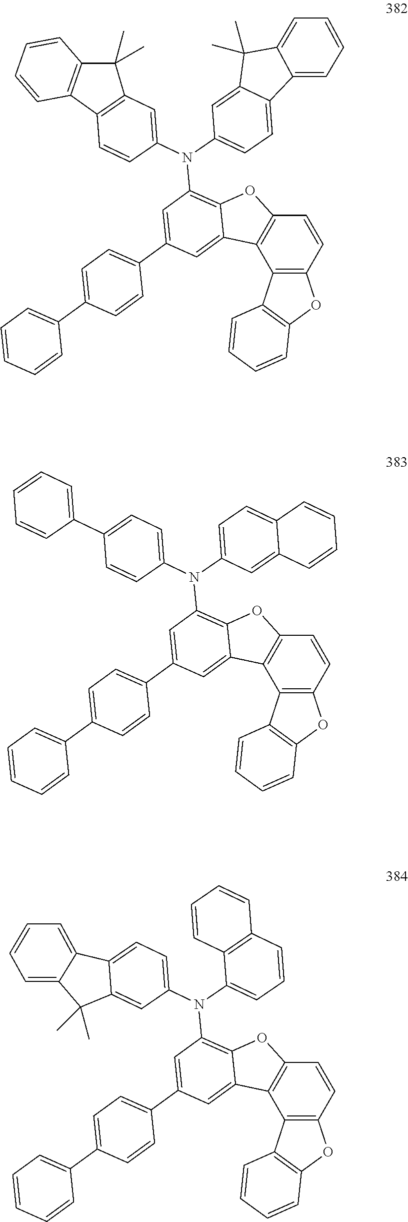 [SMILES: CC1(C)c2ccccc2-c2ccc(N(c3ccc4c(c3)C(C)(C)c3ccccc3-4)c3cc(-c4ccc(-c5ccccc5)cc4)cc4c3oc3ccc5oc6ccccc6c5c34)cc21 ; c1ccc(-c2ccc(-c3cc(N(c4ccc(-c5ccccc5)cc4)c4ccc5ccccc5c4)c4oc5ccc6oc7ccccc7c6c5c4c3)cc2)cc1 ; CC1(C)c2ccccc2-c2ccc(N(c3cccc4ccccc34)c3cc(-c4ccc(-c5ccccc5)cc4)cc4c3oc3ccc5oc6ccccc6c5c34)cc21]

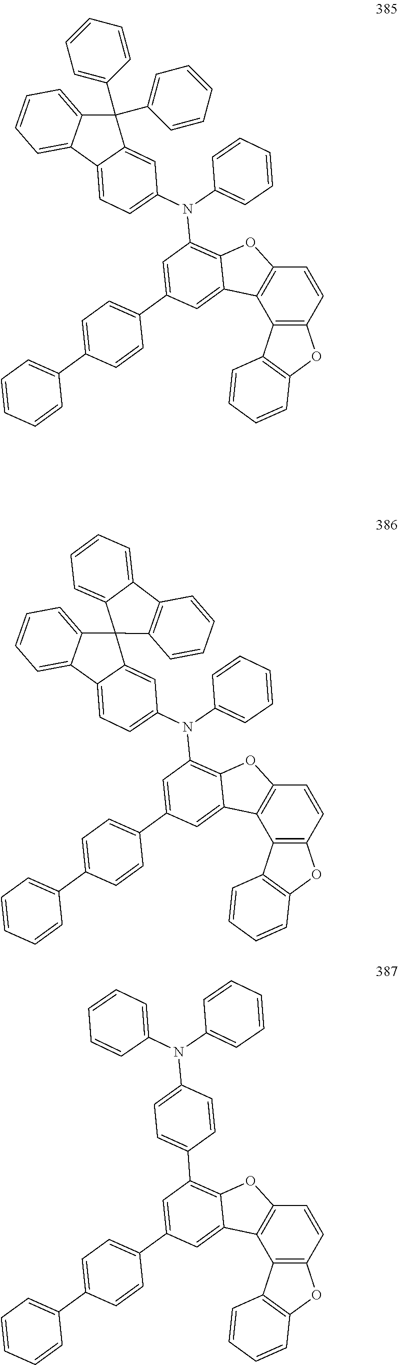 [SMILES: c1ccc(-c2ccc(-c3cc(N(c4ccccc4)c4ccc5c(c4)C(c4ccccc4)(c4ccccc4)c4ccccc4-5)c4oc5ccc6oc7ccccc7c6c5c4c3)cc2)cc1 ; c1ccc(-c2ccc(-c3cc(N(c4ccccc4)c4ccc5c(c4)C4(c6ccccc6-c6ccccc64)c4ccccc4-5)c4oc5ccc6oc7ccccc7c6c5c4c3)cc2)cc1 ; c1ccc(-c2ccc(-c3cc(-c4ccc(N(c5ccccc5)c5ccccc5)cc4)c4oc5ccc6oc7ccccc7c6c5c4c3)cc2)cc1]

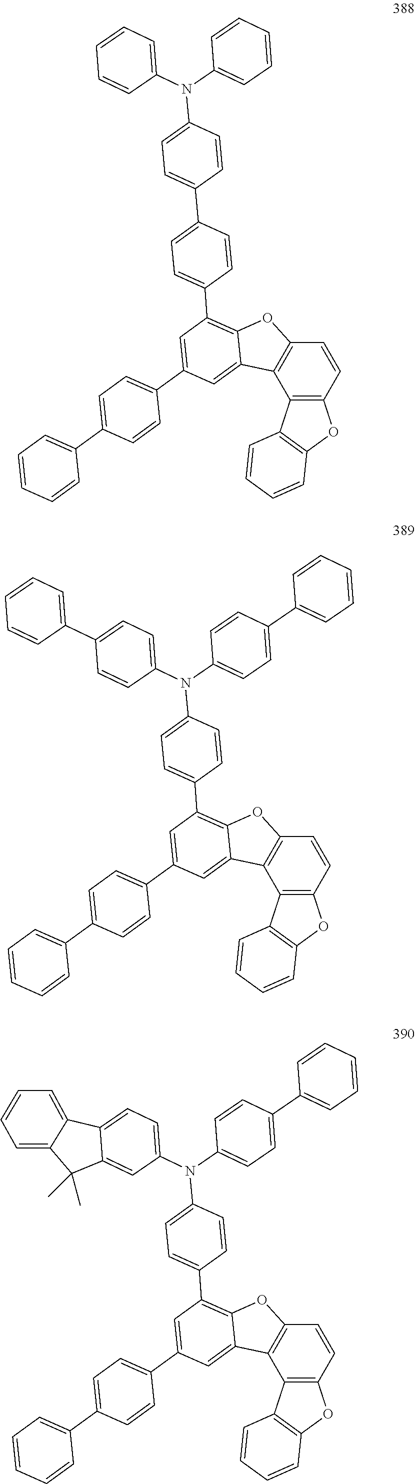 [SMILES: c1ccc(-c2ccc(-c3cc(-c4ccc(-c5ccc(N(c6ccccc6)c6ccccc6)cc5)cc4)c4oc5ccc6oc7ccccc7c6c5c4c3)cc2)cc1 ; c1ccc(-c2ccc(-c3cc(-c4ccc(N(c5ccc(-c6ccccc6)cc5)c5ccc(-c6ccccc6)cc5)cc4)c4oc5ccc6oc7ccccc7c6c5c4c3)cc2)cc1 ; CC1(C)c2ccccc2-c2ccc(N(c3ccc(-c4ccccc4)cc3)c3ccc(-c4cc(-c5ccc(-c6ccccc6)cc5)cc5c4oc4ccc6oc7ccccc7c6c45)cc3)cc21]

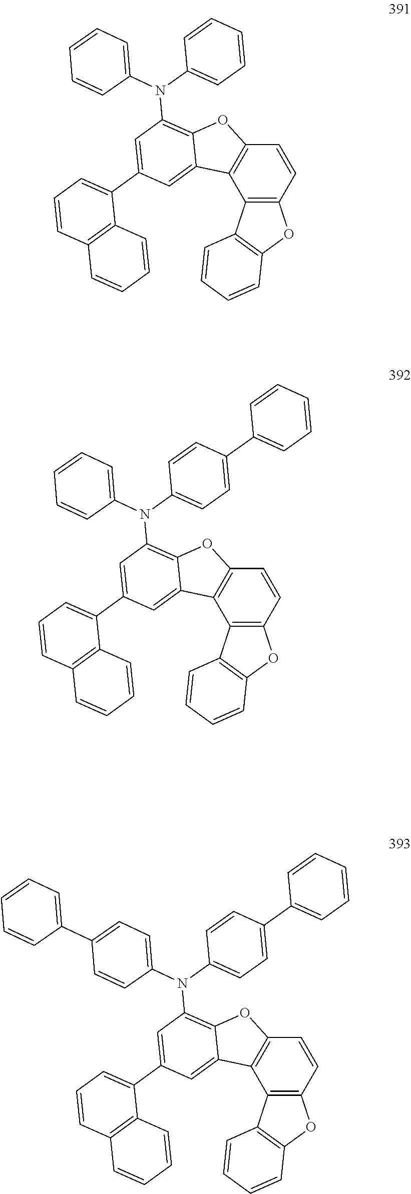 [SMILES: c1ccc(N(c2ccccc2)c2cc(-c3cccc4ccccc34)cc3c2oc2ccc4oc5ccccc5c4c23)cc1 ; c1ccc(-c2ccc(N(c3ccccc3)c3cc(-c4cccc5ccccc45)cc4c3oc3ccc5oc6ccccc6c5c34)cc2)cc1 ; c1ccc(-c2ccc(N(c3ccc(-c4ccccc4)cc3)c3cc(-c4cccc5ccccc45)cc4c3oc3ccc5oc6ccccc6c5c34)cc2)cc1]

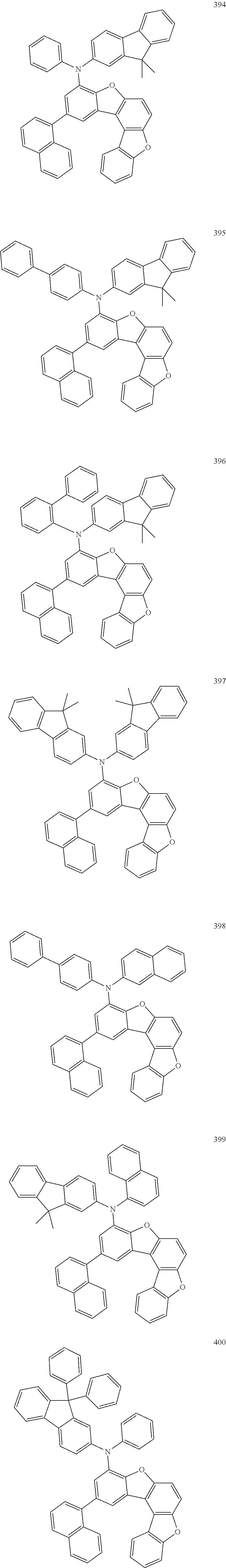

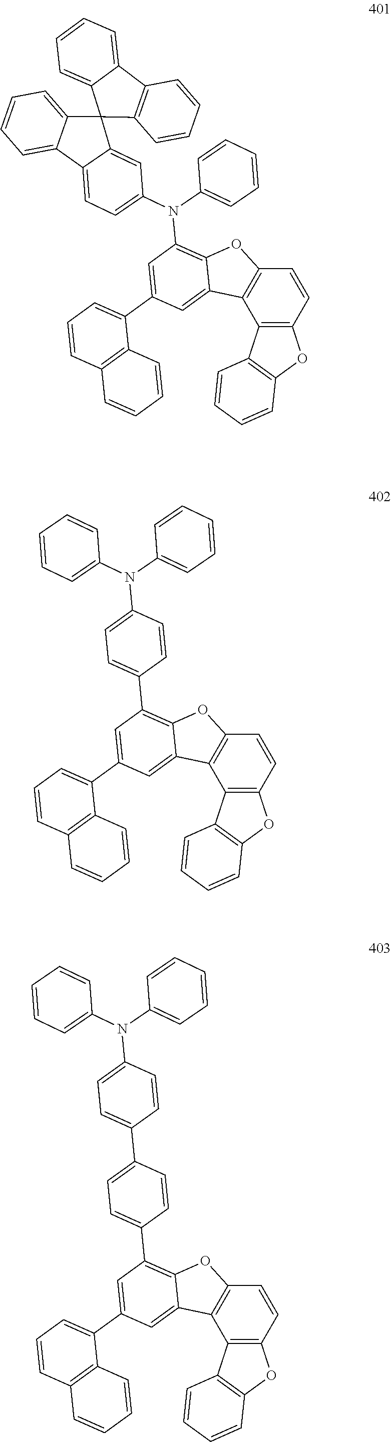 [SMILES: c1ccc(N(c2ccc3c(c2)C2(c4ccccc4-c4ccccc42)c2ccccc2-3)c2cc(-c3cccc4ccccc34)cc3c2oc2ccc4oc5ccccc5c4c23)cc1 ; c1ccc(N(c2ccccc2)c2ccc(-c3cc(-c4cccc5ccccc45)cc4c3oc3ccc5oc6ccccc6c5c34)cc2)cc1 ; c1ccc(N(c2ccccc2)c2ccc(-c3ccc(-c4cc(-c5cccc6ccccc56)cc5c4oc4ccc6oc7ccccc7c6c45)cc3)cc2)cc1]

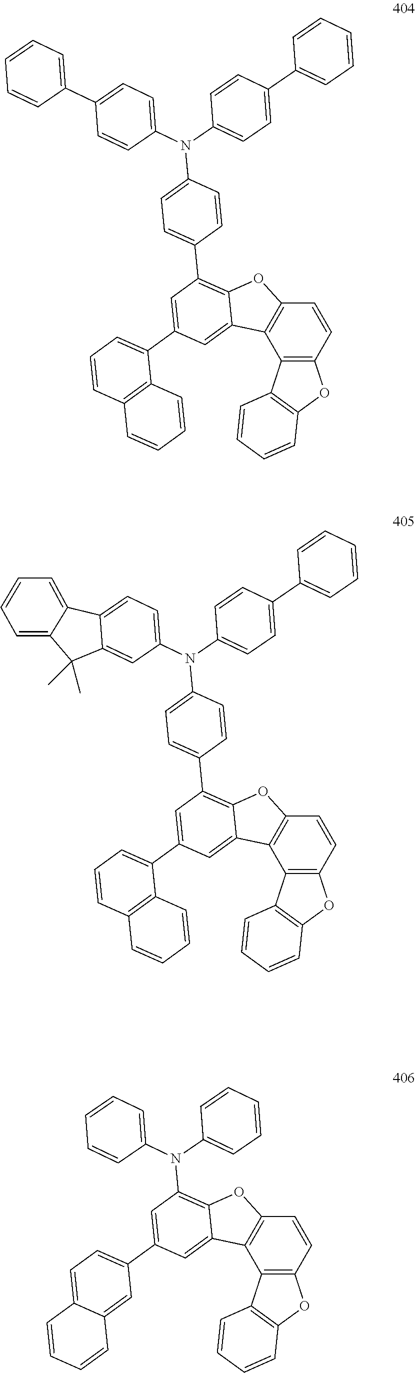 [SMILES: c1ccc(-c2ccc(N(c3ccc(-c4ccccc4)cc3)c3ccc(-c4cc(-c5cccc6ccccc56)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5cccc6ccccc56)cc5c4oc4ccc6oc7ccccc7c6c45)cc3)cc21 ; c1ccc(N(c2ccccc2)c2cc(-c3ccc4ccccc4c3)cc3c2oc2ccc4oc5ccccc5c4c23)cc1]

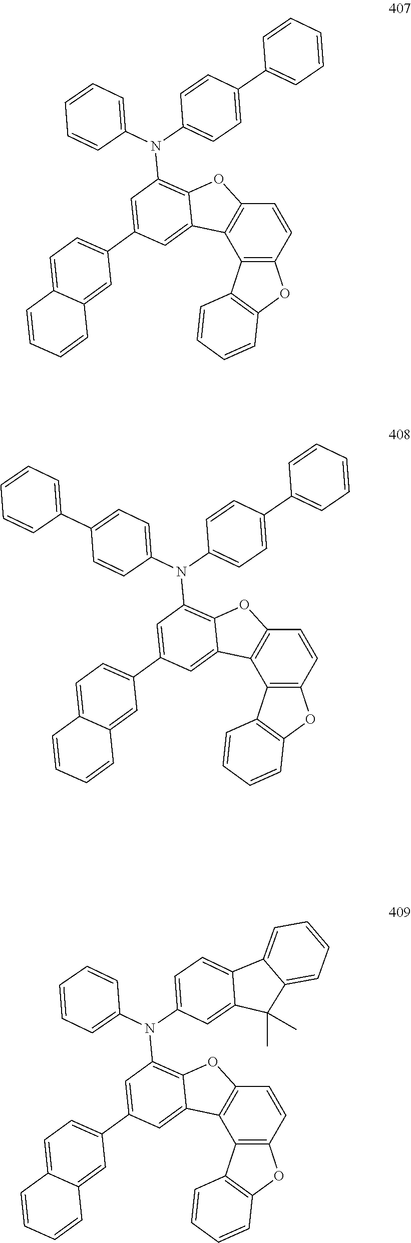 [SMILES: c1ccc(-c2ccc(N(c3ccccc3)c3cc(-c4ccc5ccccc5c4)cc4c3oc3ccc5oc6ccccc6c5c34)cc2)cc1 ; c1ccc(-c2ccc(N(c3ccc(-c4ccccc4)cc3)c3cc(-c4ccc5ccccc5c4)cc4c3oc3ccc5oc6ccccc6c5c34)cc2)cc1 ; CC1(C)c2ccccc2-c2ccc(N(c3ccccc3)c3cc(-c4ccc5ccccc5c4)cc4c3oc3ccc5oc6ccccc6c5c34)cc21]

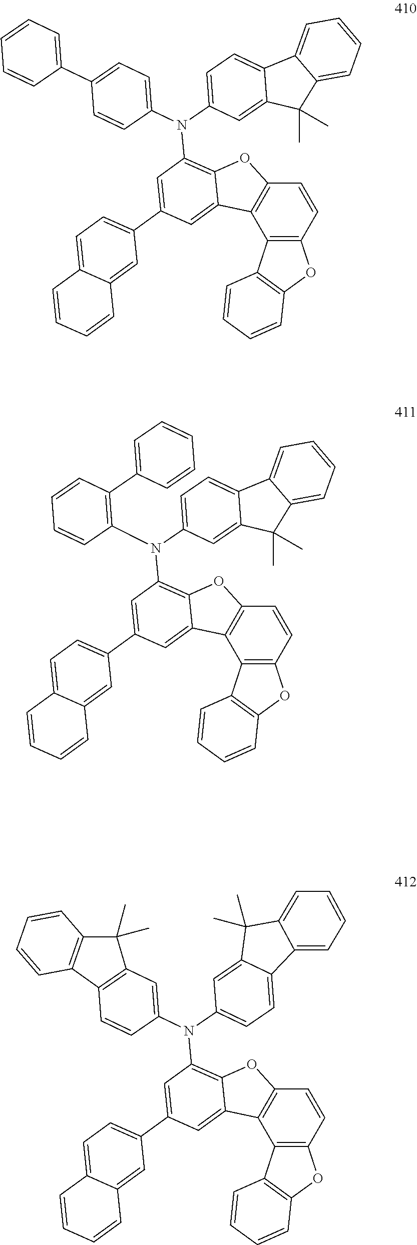 [SMILES: CC1(C)c2ccccc2-c2ccc(N(c3ccc(-c4ccccc4)cc3)c3cc(-c4ccc5ccccc5c4)cc4c3oc3ccc5oc6ccccc6c5c34)cc21 ; CC1(C)c2ccccc2-c2ccc(N(c3ccccc3-c3ccccc3)c3cc(-c4ccc5ccccc5c4)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4ccc5ccccc5c4)cc4c3oc3ccc5oc6ccccc6c5c34)cc21]

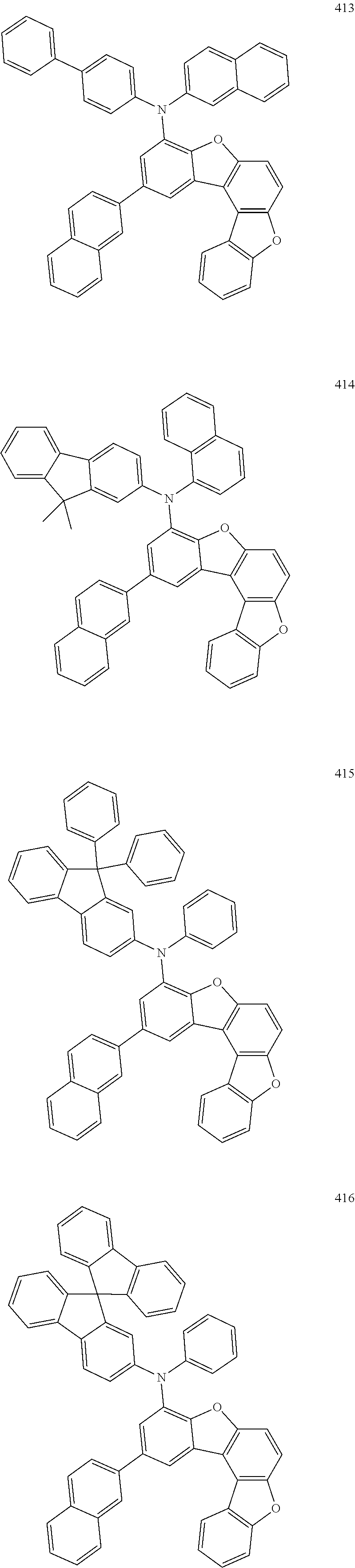 [SMILES: c1ccc(-c2ccc(N(c3ccc4ccccc4c3)c3cc(-c4ccc5ccccc5c4)cc4c3oc3ccc5oc6ccccc6c5c34)cc2)cc1 ; CC1(C)c2ccccc2-c2ccc(N(c3cccc4ccccc34)c3cc(-c4ccc5ccccc5c4)cc4c3oc3ccc5oc6ccccc6c5c34)cc21 ; c1ccc(N(c2ccc3c(c2)C(c2ccccc2)(c2ccccc2)c2ccccc2-3)c2cc(-c3ccc4ccccc4c3)cc3c2oc2ccc4oc5ccccc5c4c23)cc1 ; c1ccc(N(c2ccc3c(c2)C2(c4ccccc4-c4ccccc42)c2ccccc2-3)c2cc(-c3ccc4ccccc4c3)cc3c2oc2ccc4oc5ccccc5c4c23)cc1]

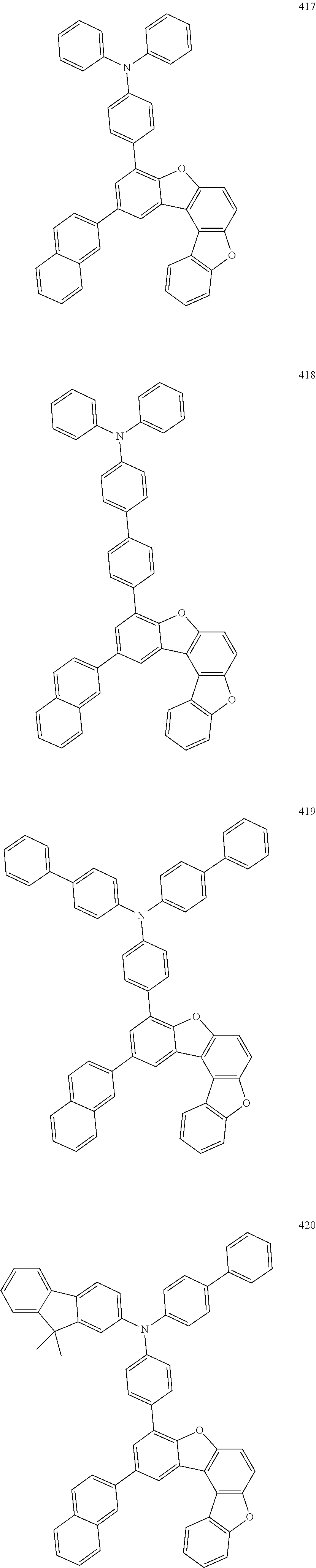 [SMILES: c1ccc(N(c2ccccc2)c2ccc(-c3cc(-c4ccc5ccccc5c4)cc4c3oc3ccc5oc6ccccc6c5c34)cc2)cc1 ; c1ccc(N(c2ccccc2)c2ccc(-c3ccc(-c4cc(-c5ccc6ccccc6c5)cc5c4oc4ccc6oc7ccccc7c6c45)cc3)cc2)cc1 ; c1ccc(-c2ccc(N(c3ccc(-c4ccccc4)cc3)c3ccc(-c4cc(-c5ccc6ccccc6c5)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5ccc6ccccc6c5)cc5c4oc4ccc6oc7ccccc7c6c45)cc3)cc21]

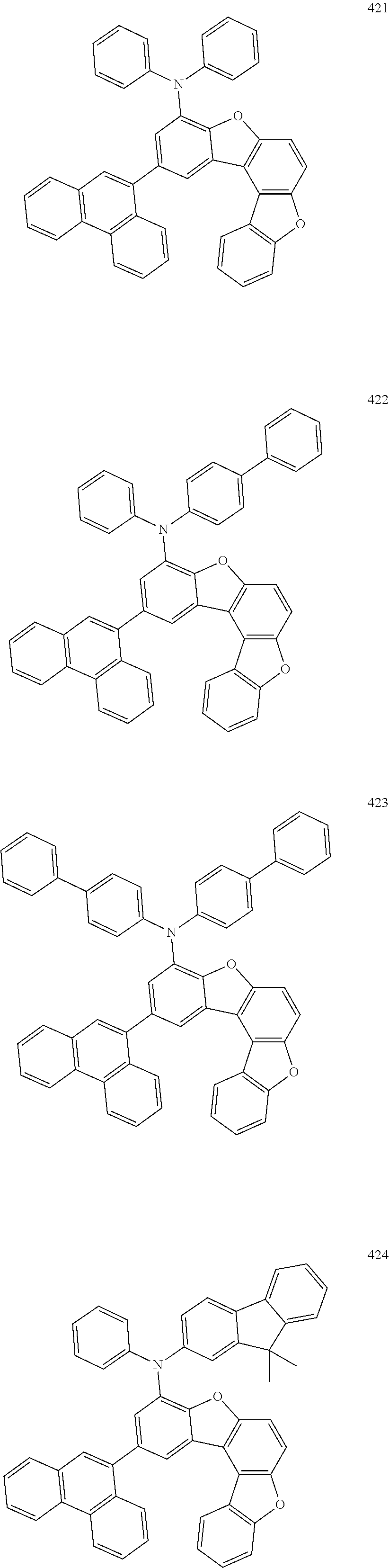 [SMILES: c1ccc(N(c2ccccc2)c2cc(-c3cc4ccccc4c4ccccc34)cc3c2oc2ccc4oc5ccccc5c4c23)cc1 ; c1ccc(-c2ccc(N(c3ccccc3)c3cc(-c4cc5ccccc5c5ccccc45)cc4c3oc3ccc5oc6ccccc6c5c34)cc2)cc1 ; c1ccc(-c2ccc(N(c3ccc(-c4ccccc4)cc3)c3cc(-c4cc5ccccc5c5ccccc45)cc4c3oc3ccc5oc6ccccc6c5c34)cc2)cc1 ; CC1(C)c2ccccc2-c2ccc(N(c3ccccc3)c3cc(-c4cc5ccccc5c5ccccc45)cc4c3oc3ccc5oc6ccccc6c5c34)cc21]

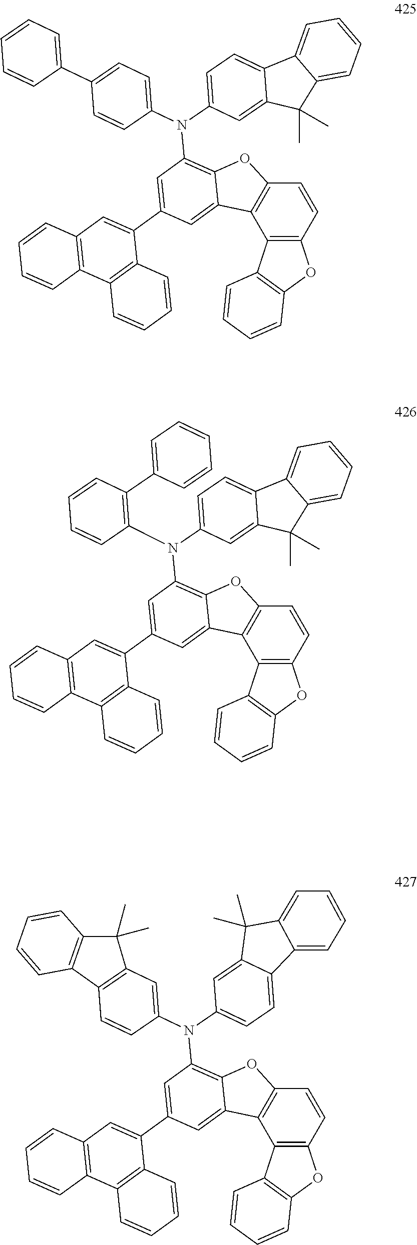 [SMILES: CC1(C)c2ccccc2-c2ccc(N(c3ccc(-c4ccccc4)cc3)c3cc(-c4cc5ccccc5c5ccccc45)cc4c3oc3ccc5oc6ccccc6c5c34)cc21 ; CC1(C)c2ccccc2-c2ccc(N(c3ccccc3-c3ccccc3)c3cc(-c4cc5ccccc5c5ccccc45)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4cc5ccccc5c5ccccc45)cc4c3oc3ccc5oc6ccccc6c5c34)cc21]

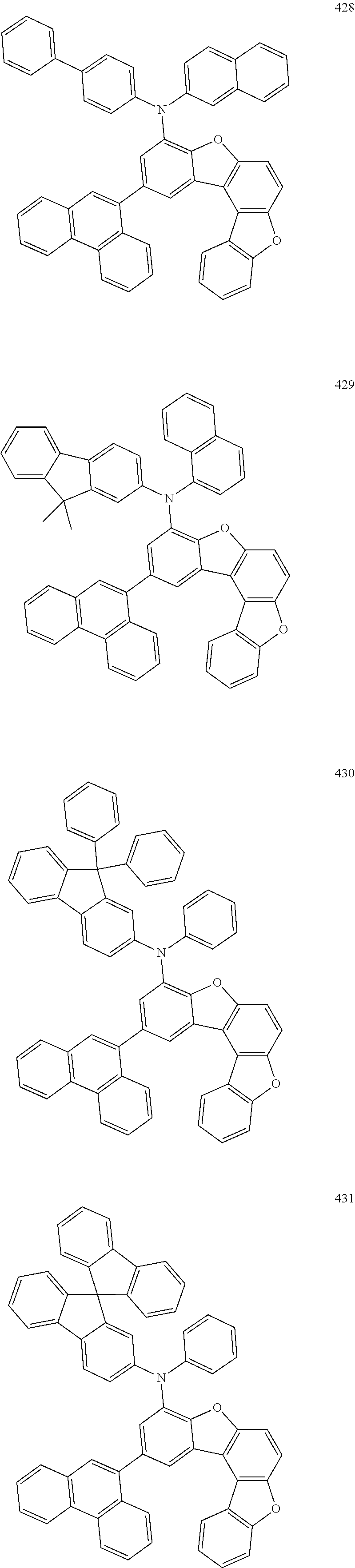 [SMILES: c1ccc(-c2ccc(N(c3ccc4ccccc4c3)c3cc(-c4cc5ccccc5c5ccccc45)cc4c3oc3ccc5oc6ccccc6c5c34)cc2)cc1 ; CC1(C)c2ccccc2-c2ccc(N(c3cccc4ccccc34)c3cc(-c4cc5ccccc5c5ccccc45)cc4c3oc3ccc5oc6ccccc6c5c34)cc21 ; c1ccc(N(c2ccc3c(c2)C(c2ccccc2)(c2ccccc2)c2ccccc2-3)c2cc(-c3cc4ccccc4c4ccccc34)cc3c2oc2ccc4oc5ccccc5c4c23)cc1 ; c1ccc(N(c2ccc3c(c2)C2(c4ccccc4-c4ccccc42)c2ccccc2-3)c2cc(-c3cc4ccccc4c4ccccc34)cc3c2oc2ccc4oc5ccccc5c4c23)cc1]

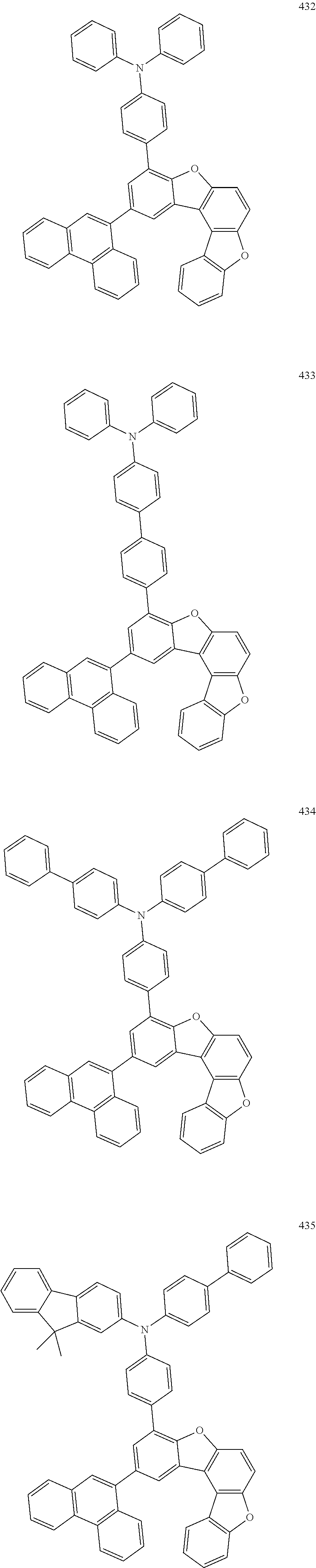 [SMILES: c1ccc(N(c2ccccc2)c2ccc(-c3cc(-c4cc5ccccc5c5ccccc45)cc4c3oc3ccc5oc6ccccc6c5c34)cc2)cc1 ; c1ccc(N(c2ccccc2)c2ccc(-c3ccc(-c4cc(-c5cc6ccccc6c6ccccc56)cc5c4oc4ccc6oc7ccccc7c6c45)cc3)cc2)cc1 ; c1ccc(-c2ccc(N(c3ccc(-c4ccccc4)cc3)c3ccc(-c4cc(-c5cc6ccccc6c6ccccc56)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5cc6ccccc6c6ccccc56)cc5c4oc4ccc6oc7ccccc7c6c45)cc3)cc21]

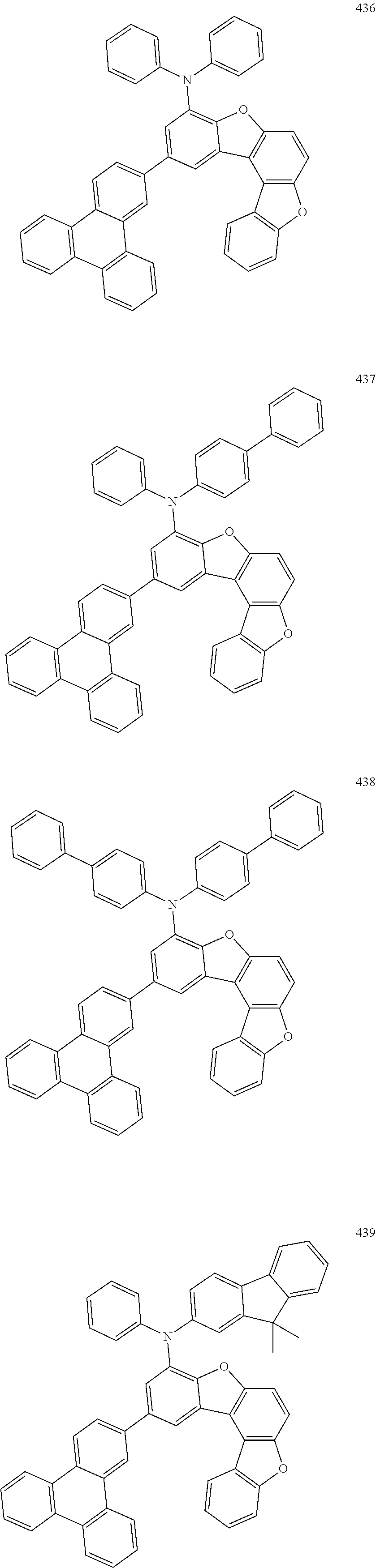 [SMILES: c1ccc(N(c2ccccc2)c2cc(-c3ccc4c5ccccc5c5ccccc5c4c3)cc3c2oc2ccc4oc5ccccc5c4c23)cc1 ; c1ccc(-c2ccc(N(c3ccccc3)c3cc(-c4ccc5c6ccccc6c6ccccc6c5c4)cc4c3oc3ccc5oc6ccccc6c5c34)cc2)cc1 ; c1ccc(-c2ccc(N(c3ccc(-c4ccccc4)cc3)c3cc(-c4ccc5c6ccccc6c6ccccc6c5c4)cc4c3oc3ccc5oc6ccccc6c5c34)cc2)cc1 ; CC1(C)c2ccccc2-c2ccc(N(c3ccccc3)c3cc(-c4ccc5c6ccccc6c6ccccc6c5c4)cc4c3oc3ccc5oc6ccccc6c5c34)cc21]

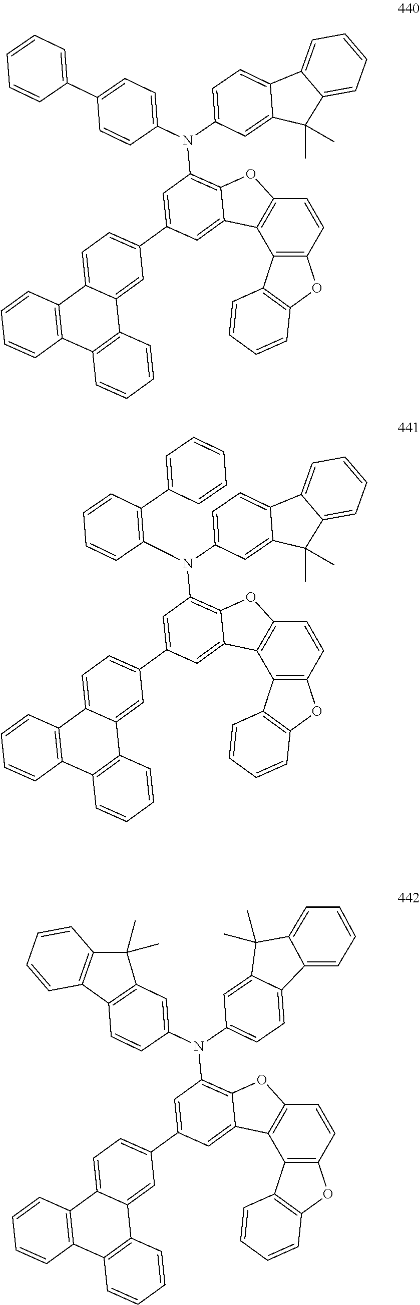 [SMILES: CC1(C)c2ccccc2-c2ccc(N(c3ccc(-c4ccccc4)cc3)c3cc(-c4ccc5c6ccccc6c6ccccc6c5c4)cc4c3oc3ccc5oc6ccccc6c5c34)cc21 ; CC1(C)c2ccccc2-c2ccc(N(c3ccccc3-c3ccccc3)c3cc(-c4ccc5c6ccccc6c6ccccc6c5c4)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4ccc5c6ccccc6c6ccccc6c5c4)cc4c3oc3ccc5oc6ccccc6c5c34)cc21]

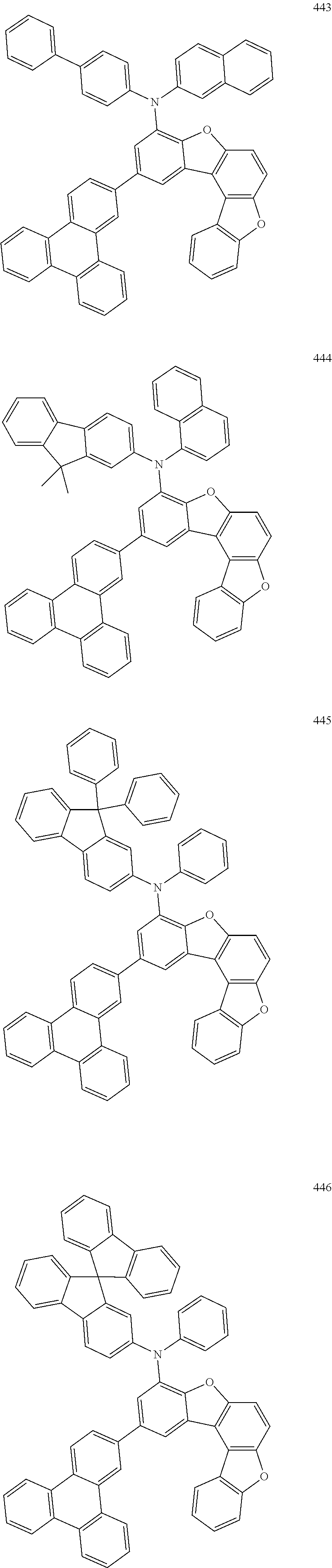 [SMILES: c1ccc(-c2ccc(N(c3ccc4ccccc4c3)c3cc(-c4ccc5c6ccccc6c6ccccc6c5c4)cc4c3oc3ccc5oc6ccccc6c5c34)cc2)cc1 ; CC1(C)c2ccccc2-c2ccc(N(c3cccc4ccccc34)c3cc(-c4ccc5c6ccccc6c6ccccc6c5c4)cc4c3oc3ccc5oc6ccccc6c5c34)cc21 ; c1ccc(N(c2ccc3c(c2)C(c2ccccc2)(c2ccccc2)c2ccccc2-3)c2cc(-c3ccc4c5ccccc5c5ccccc5c4c3)cc3c2oc2ccc4oc5ccccc5c4c23)cc1 ; c1ccc(N(c2ccc3c(c2)C2(c4ccccc4-c4ccccc42)c2ccccc2-3)c2cc(-c3ccc4c5ccccc5c5ccccc5c4c3)cc3c2oc2ccc4oc5ccccc5c4c23)cc1]

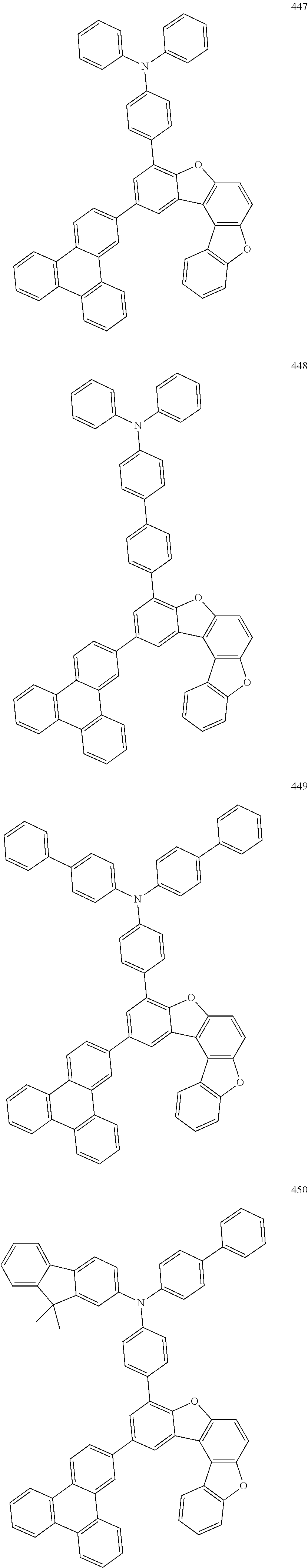 [SMILES: c1ccc(N(c2ccccc2)c2ccc(-c3cc(-c4ccc5c6ccccc6c6ccccc6c5c4)cc4c3oc3ccc5oc6ccccc6c5c34)cc2)cc1 ; c1ccc(N(c2ccccc2)c2ccc(-c3ccc(-c4cc(-c5ccc6c7ccccc7c7ccccc7c6c5)cc5c4oc4ccc6oc7ccccc7c6c45)cc3)cc2)cc1 ; c1ccc(-c2ccc(N(c3ccc(-c4ccccc4)cc3)c3ccc(-c4cc(-c5ccc6c7ccccc7c7ccccc7c6c5)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5ccc6c7ccccc7c7ccccc7c6c5)cc5c4oc4ccc6oc7ccccc7c6c45)cc3)cc21]

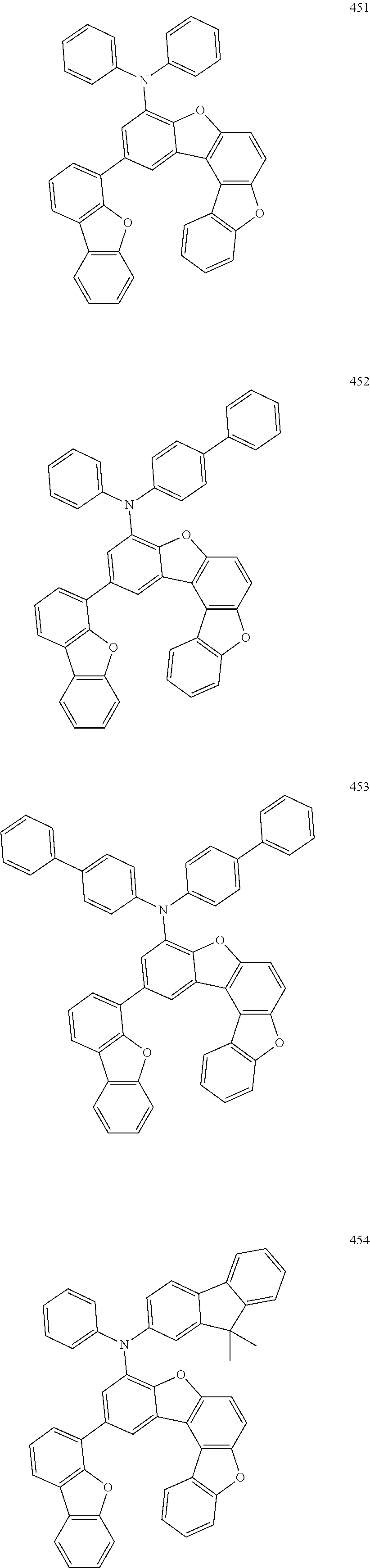 [SMILES: c1ccc(N(c2ccccc2)c2cc(-c3cccc4c3oc3ccccc34)cc3c2oc2ccc4oc5ccccc5c4c23)cc1 ; c1ccc(-c2ccc(N(c3ccccc3)c3cc(-c4cccc5c4oc4ccccc45)cc4c3oc3ccc5oc6ccccc6c5c34)cc2)cc1 ; c1ccc(-c2ccc(N(c3ccc(-c4ccccc4)cc3)c3cc(-c4cccc5c4oc4ccccc45)cc4c3oc3ccc5oc6ccccc6c5c34)cc2)cc1 ; CC1(C)c2ccccc2-c2ccc(N(c3ccccc3)c3cc(-c4cccc5c4oc4ccccc45)cc4c3oc3ccc5oc6ccccc6c5c34)cc21]

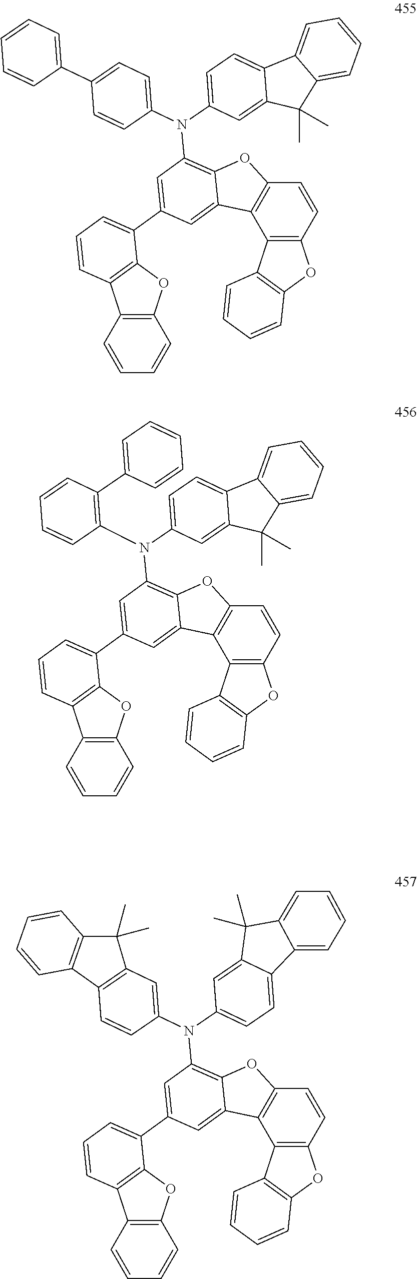 [SMILES: CC1(C)c2ccccc2-c2ccc(N(c3ccc(-c4ccccc4)cc3)c3cc(-c4cccc5c4oc4ccccc45)cc4c3oc3ccc5oc6ccccc6c5c34)cc21 ; CC1(C)c2ccccc2-c2ccc(N(c3ccccc3-c3ccccc3)c3cc(-c4cccc5c4oc4ccccc45)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4cccc5c4oc4ccccc45)cc4c3oc3ccc5oc6ccccc6c5c34)cc21]

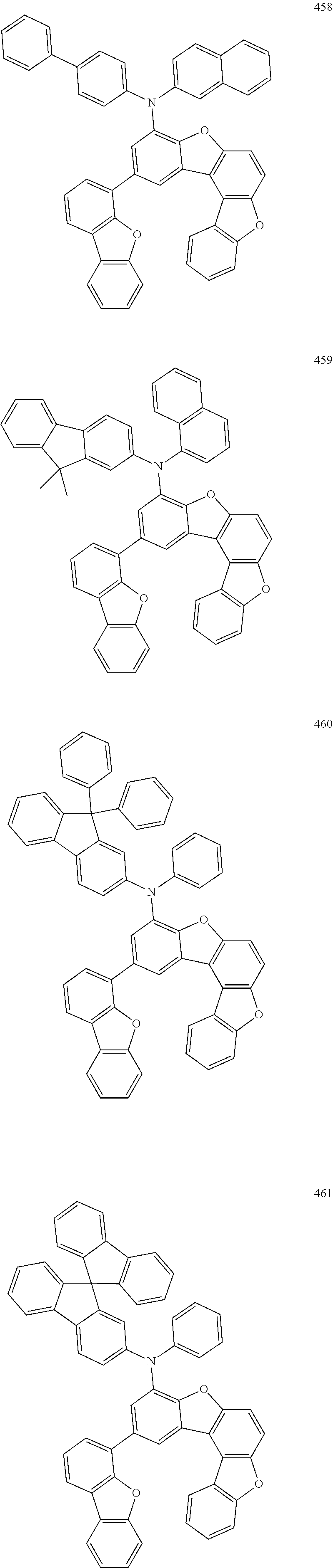 [SMILES: c1ccc(-c2ccc(N(c3ccc4ccccc4c3)c3cc(-c4cccc5c4oc4ccccc45)cc4c3oc3ccc5oc6ccccc6c5c34)cc2)cc1 ; CC1(C)c2ccccc2-c2ccc(N(c3cccc4ccccc34)c3cc(-c4cccc5c4oc4ccccc45)cc4c3oc3ccc5oc6ccccc6c5c34)cc21 ; c1ccc(N(c2ccc3c(c2)C(c2ccccc2)(c2ccccc2)c2ccccc2-3)c2cc(-c3cccc4c3oc3ccccc34)cc3c2oc2ccc4oc5ccccc5c4c23)cc1 ; c1ccc(N(c2ccc3c(c2)C2(c4ccccc4-c4ccccc42)c2ccccc2-3)c2cc(-c3cccc4c3oc3ccccc34)cc3c2oc2ccc4oc5ccccc5c4c23)cc1]

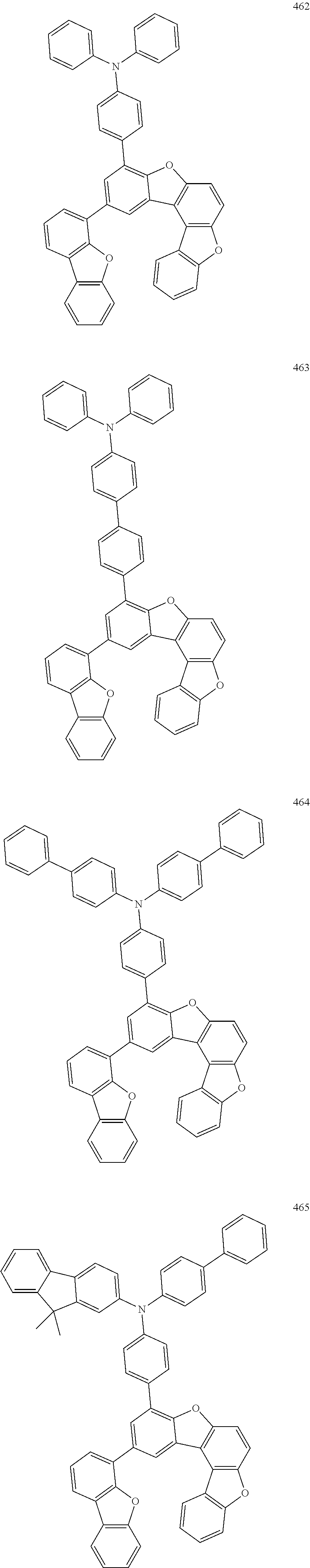 [SMILES: c1ccc(N(c2ccccc2)c2ccc(-c3cc(-c4cccc5c4oc4ccccc45)cc4c3oc3ccc5oc6ccccc6c5c34)cc2)cc1 ; c1ccc(N(c2ccccc2)c2ccc(-c3ccc(-c4cc(-c5cccc6c5oc5ccccc56)cc5c4oc4ccc6oc7ccccc7c6c45)cc3)cc2)cc1 ; c1ccc(-c2ccc(N(c3ccc(-c4ccccc4)cc3)c3ccc(-c4cc(-c5cccc6c5oc5ccccc56)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5cccc6c5oc5ccccc56)cc5c4oc4ccc6oc7ccccc7c6c45)cc3)cc21]

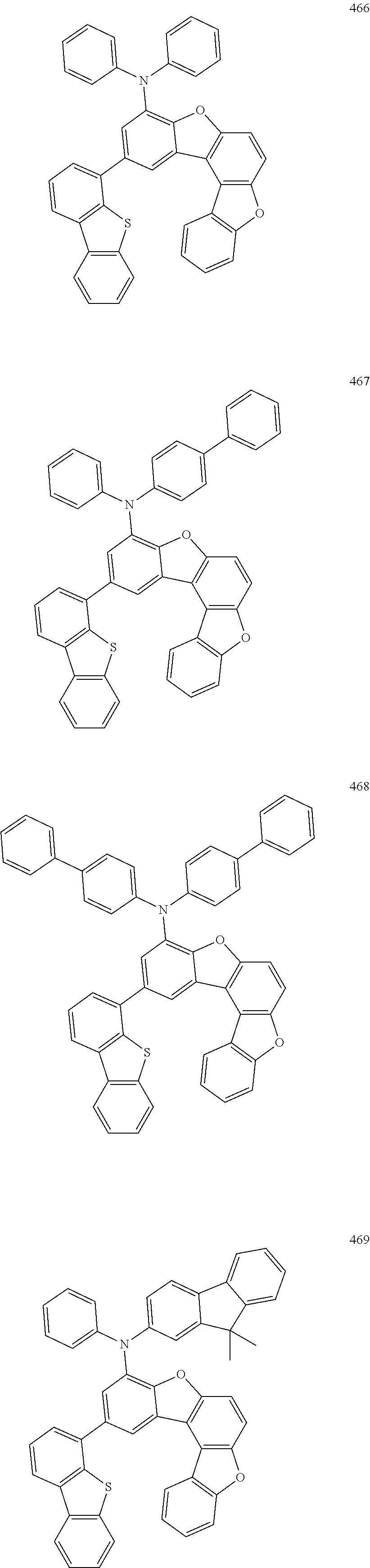 [SMILES: c1ccc(N(c2ccccc2)c2cc(-c3cccc4c3sc3ccccc34)cc3c2oc2ccc4oc5ccccc5c4c23)cc1 ; c1ccc(-c2ccc(N(c3ccccc3)c3cc(-c4cccc5c4sc4ccccc45)cc4c3oc3ccc5oc6ccccc6c5c34)cc2)cc1 ; c1ccc(-c2ccc(N(c3ccc(-c4ccccc4)cc3)c3cc(-c4cccc5c4sc4ccccc45)cc4c3oc3ccc5oc6ccccc6c5c34)cc2)cc1 ; CC1(C)c2ccccc2-c2ccc(N(c3ccccc3)c3cc(-c4cccc5c4sc4ccccc45)cc4c3oc3ccc5oc6ccccc6c5c34)cc21]

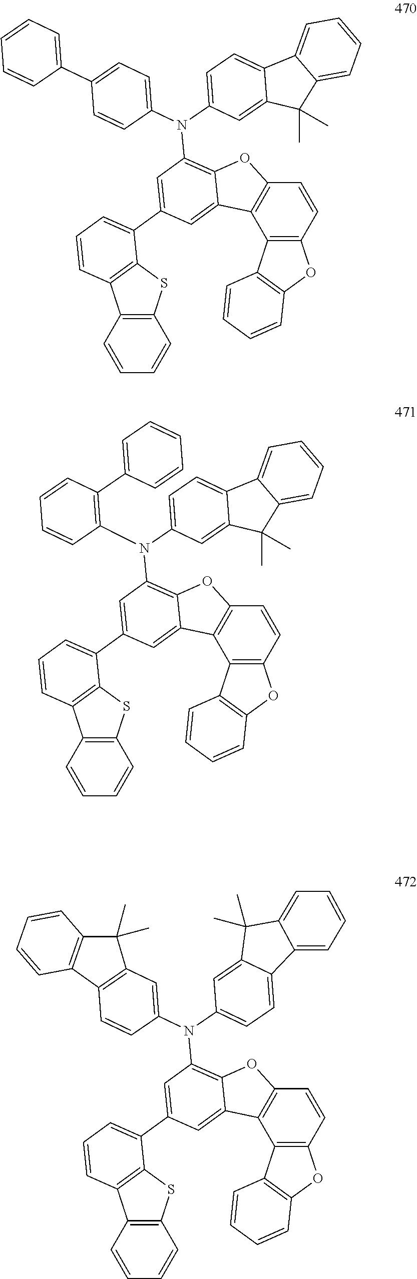 [SMILES: CC1(C)c2ccccc2-c2ccc(N(c3ccc(-c4ccccc4)cc3)c3cc(-c4cccc5c4sc4ccccc45)cc4c3oc3ccc5oc6ccccc6c5c34)cc21 ; CC1(C)c2ccccc2-c2ccc(N(c3ccccc3-c3ccccc3)c3cc(-c4cccc5c4sc4ccccc45)cc4c3oc3ccc5oc6ccccc6c5c34)cc21 ; CC1(C)c2ccccc2-c2ccc(N(c3ccc4c(c3)C(C)(C)c3ccccc3-4)c3cc(-c4cccc5c4sc4ccccc45)cc4c3oc3ccc5oc6ccccc6c5c34)cc21]

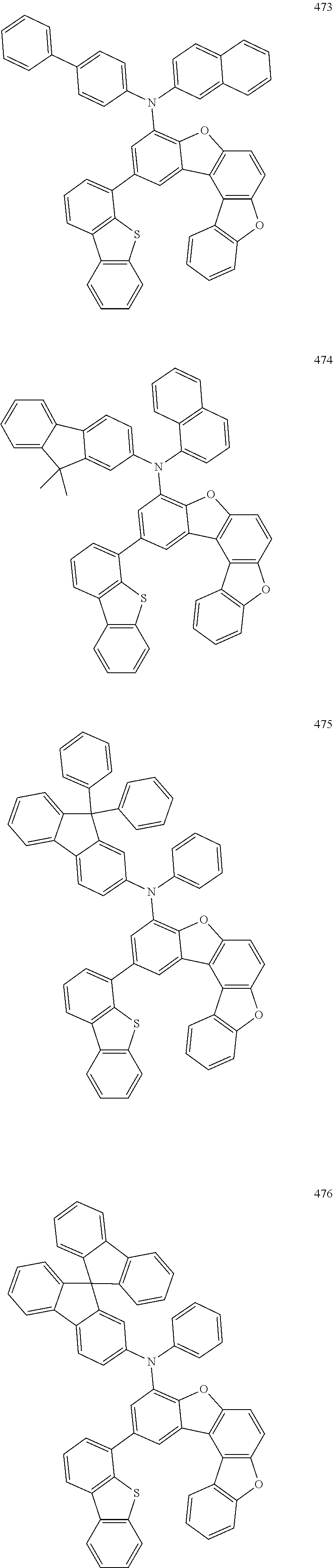 [SMILES: c1ccc(-c2ccc(N(c3ccc4ccccc4c3)c3cc(-c4cccc5c4sc4ccccc45)cc4c3oc3ccc5oc6ccccc6c5c34)cc2)cc1 ; CC1(C)c2ccccc2-c2ccc(N(c3cccc4ccccc34)c3cc(-c4cccc5c4sc4ccccc45)cc4c3oc3ccc5oc6ccccc6c5c34)cc21 ; c1ccc(N(c2ccc3c(c2)C(c2ccccc2)(c2ccccc2)c2ccccc2-3)c2cc(-c3cccc4c3sc3ccccc34)cc3c2oc2ccc4oc5ccccc5c4c23)cc1 ; c1ccc(N(c2ccc3c(c2)C2(c4ccccc4-c4ccccc42)c2ccccc2-3)c2cc(-c3cccc4c3sc3ccccc34)cc3c2oc2ccc4oc5ccccc5c4c23)cc1]

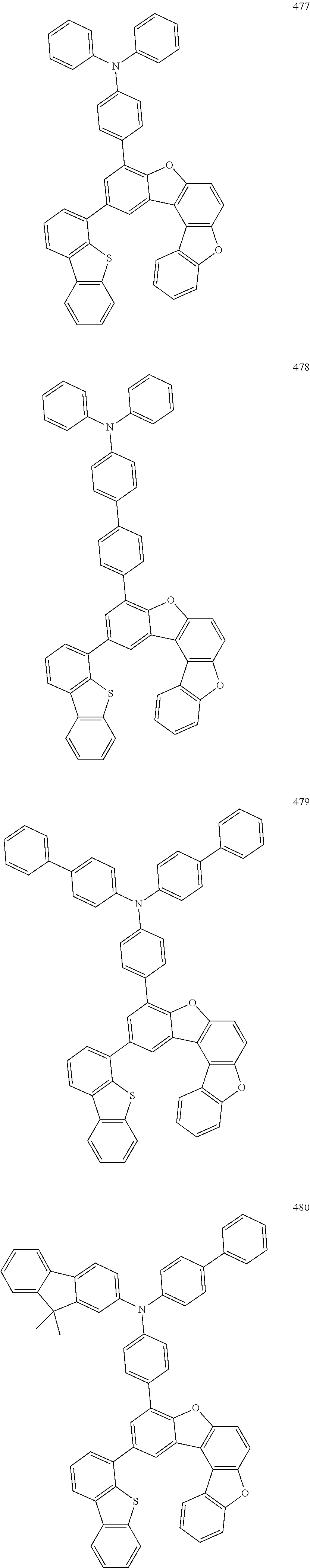 [SMILES: c1ccc(N(c2ccccc2)c2ccc(-c3cc(-c4cccc5c4sc4ccccc45)cc4c3oc3ccc5oc6ccccc6c5c34)cc2)cc1 ; c1ccc(N(c2ccccc2)c2ccc(-c3ccc(-c4cc(-c5cccc6c5sc5ccccc56)cc5c4oc4ccc6oc7ccccc7c6c45)cc3)cc2)cc1 ; c1ccc(-c2ccc(N(c3ccc(-c4ccccc4)cc3)c3ccc(-c4cc(-c5cccc6c5sc5ccccc56)cc5c4oc4ccc6oc7ccccc7c6c45)cc3)cc2)cc1 ; CC1(C)c2ccccc2-c2ccc(N(c3ccc(-c4ccccc4)cc3)c3ccc(-c4cc(-c5cccc6c5sc5ccccc56)cc5c4oc4ccc6oc7ccccc7c6c45)cc3)cc21]

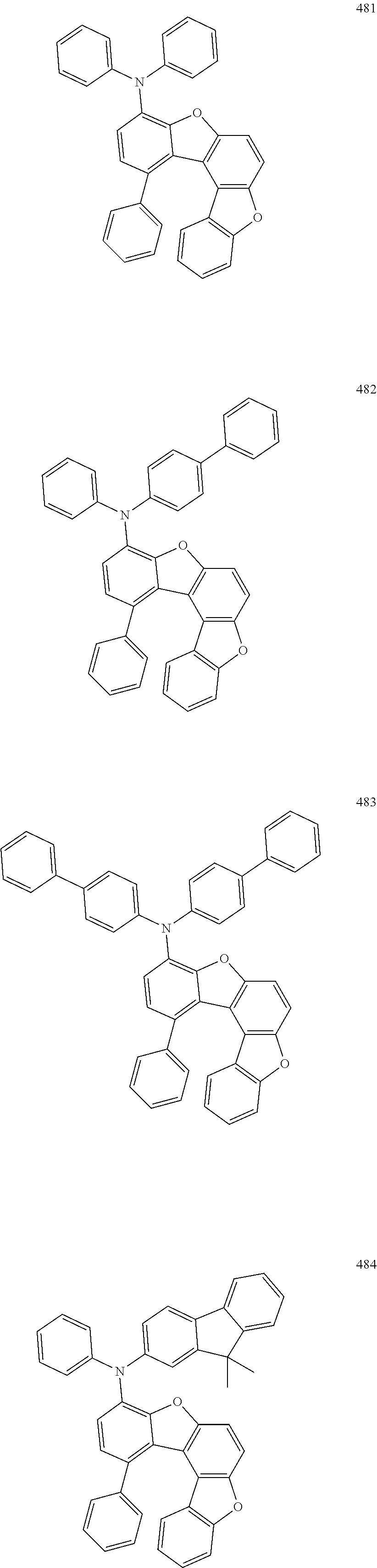 [SMILES: c1ccc(-c2ccc(N(c3ccccc3)c3ccccc3)c3oc4ccc5oc6ccccc6c5c4c23)cc1 ; c1ccc(-c2ccc(N(c3ccccc3)c3ccc(-c4ccccc4)c4c3oc3ccc5oc6ccccc6c5c34)cc2)cc1 ; c1ccc(-c2ccc(N(c3ccc(-c4ccccc4)cc3)c3ccc(-c4ccccc4)c4c3oc3ccc5oc6ccccc6c5c34)cc2)cc1 ; CC1(C)c2ccccc2-c2ccc(N(c3ccccc3)c3ccc(-c4ccccc4)c4c3oc3ccc5oc6ccccc6c5c34)cc21]

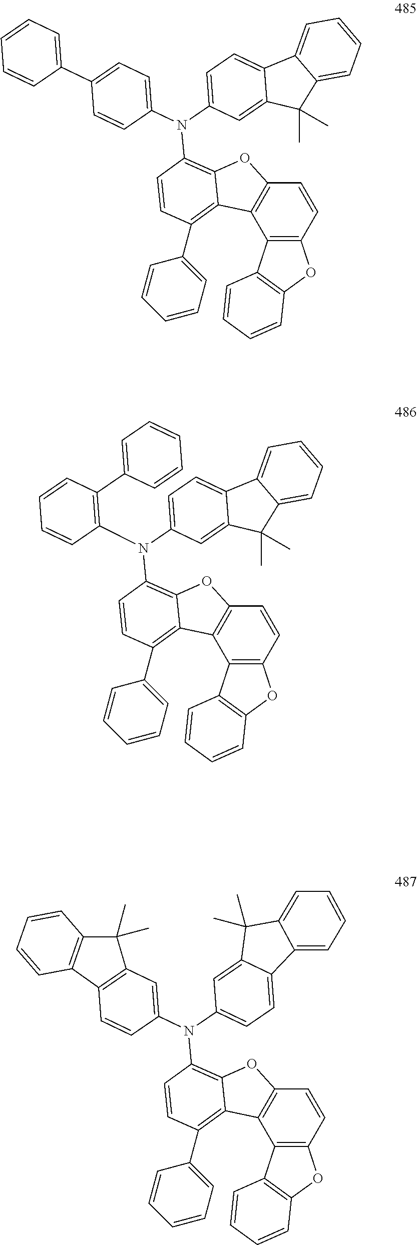 [SMILES: CC1(C)c2ccccc2-c2ccc(N(c3ccc(-c4ccccc4)cc3)c3ccc(-c4ccccc4)c4c3oc3ccc5oc6ccccc6c5c34)cc21 ; CC1(C)c2ccccc2-c2ccc(N(c3ccccc3-c3ccccc3)c3ccc(-c4ccccc4)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4ccccc4)c4c3oc3ccc5oc6ccccc6c5c34)cc21]

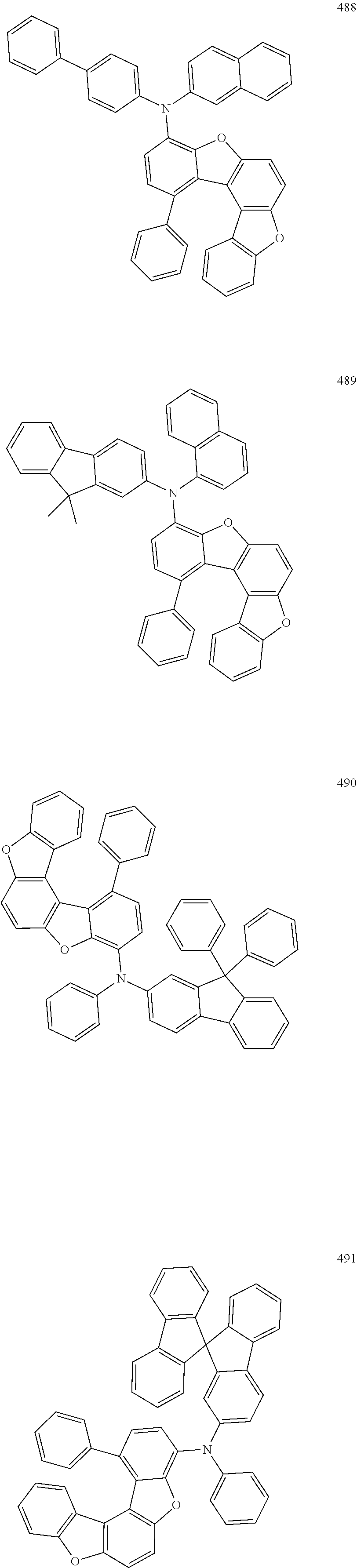 [SMILES: c1ccc(-c2ccc(N(c3ccc4ccccc4c3)c3ccc(-c4ccccc4)c4c3oc3ccc5oc6ccccc6c5c34)cc2)cc1 ; CC1(C)c2ccccc2-c2ccc(N(c3cccc4ccccc34)c3ccc(-c4ccccc4)c4c3oc3ccc5oc6ccccc6c5c34)cc21 ; c1ccc(-c2ccc(N(c3ccccc3)c3ccc4c(c3)C(c3ccccc3)(c3ccccc3)c3ccccc3-4)c3oc4ccc5oc6ccccc6c5c4c23)cc1 ; c1ccc(-c2ccc(N(c3ccccc3)c3ccc4c(c3)C3(c5ccccc5-c5ccccc53)c3ccccc3-4)c3oc4ccc5oc6ccccc6c5c4c23)cc1]

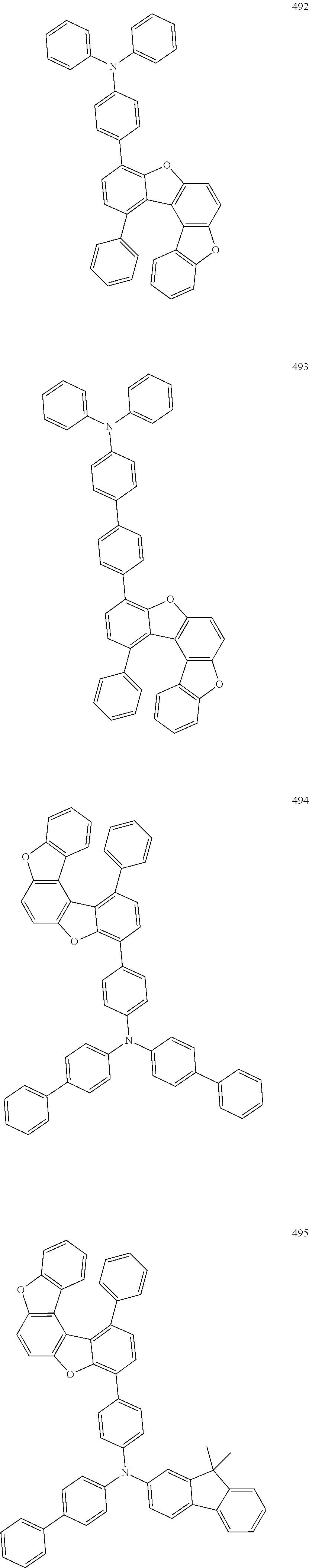 [SMILES: c1ccc(-c2ccc(-c3ccc(N(c4ccccc4)c4ccccc4)cc3)c3oc4ccc5oc6ccccc6c5c4c23)cc1 ; c1ccc(-c2ccc(-c3ccc(-c4ccc(N(c5ccccc5)c5ccccc5)cc4)cc3)c3oc4ccc5oc6ccccc6c5c4c23)cc1 ; c1ccc(-c2ccc(N(c3ccc(-c4ccccc4)cc3)c3ccc(-c4ccc(-c5ccccc5)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5ccccc5)c5c4oc4ccc6oc7ccccc7c6c45)cc3)cc21]

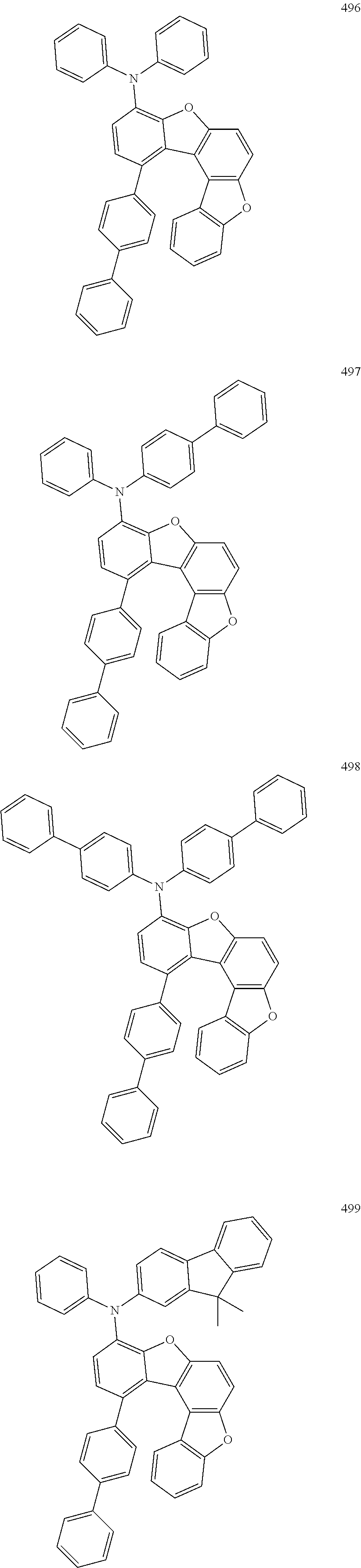 [SMILES: c1ccc(-c2ccc(-c3ccc(N(c4ccccc4)c4ccccc4)c4oc5ccc6oc7ccccc7c6c5c34)cc2)cc1 ; c1ccc(-c2ccc(-c3ccc(N(c4ccccc4)c4ccc(-c5ccccc5)cc4)c4oc5ccc6oc7ccccc7c6c5c34)cc2)cc1 ; c1ccc(-c2ccc(-c3ccc(N(c4ccc(-c5ccccc5)cc4)c4ccc(-c5ccccc5)cc4)c4oc5ccc6oc7ccccc7c6c5c34)cc2)cc1 ; CC1(C)c2ccccc2-c2ccc(N(c3ccccc3)c3ccc(-c4ccc(-c5ccccc5)cc4)c4c3oc3ccc5oc6ccccc6c5c34)cc21]

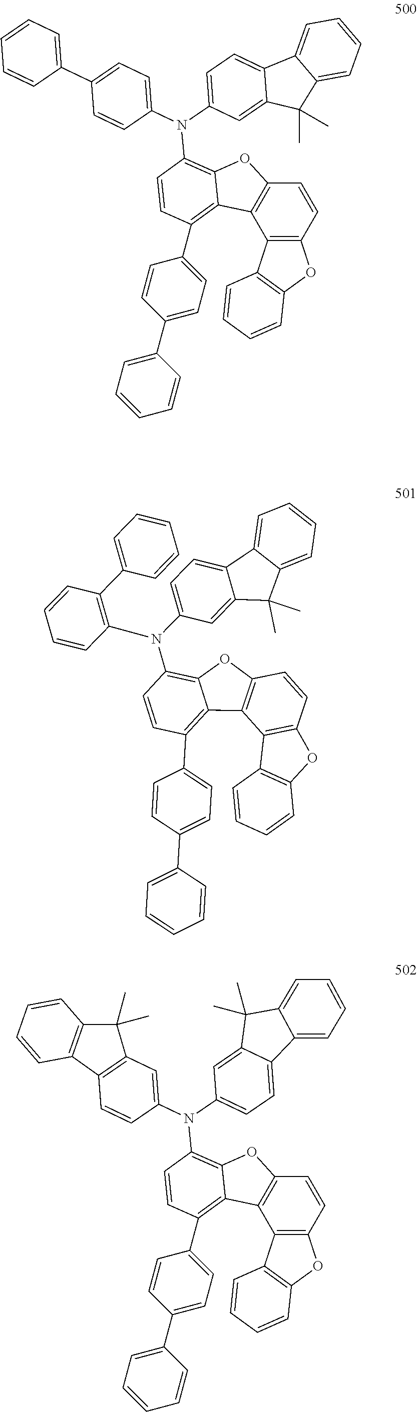 [SMILES: CC1(C)c2ccccc2-c2ccc(N(c3ccc(-c4ccccc4)cc3)c3ccc(-c4ccc(-c5ccccc5)cc4)c4c3oc3ccc5oc6ccccc6c5c34)cc21 ; CC1(C)c2ccccc2-c2ccc(N(c3ccccc3-c3ccccc3)c3ccc(-c4ccc(-c5ccccc5)cc4)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4ccc(-c5ccccc5)cc4)c4c3oc3ccc5oc6ccccc6c5c34)cc21]

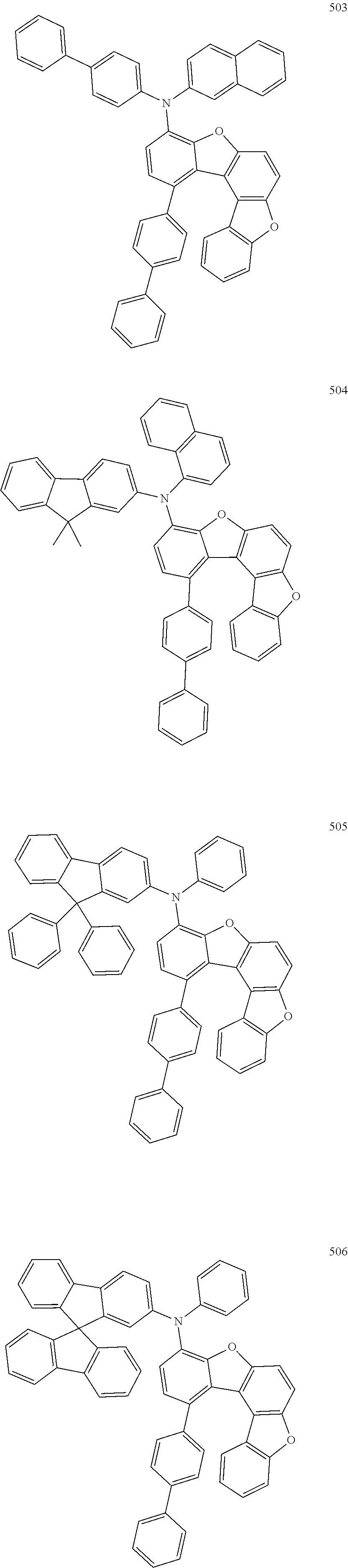 [SMILES: c1ccc(-c2ccc(-c3ccc(N(c4ccc(-c5ccccc5)cc4)c4ccc5ccccc5c4)c4oc5ccc6oc7ccccc7c6c5c34)cc2)cc1 ; CC1(C)c2ccccc2-c2ccc(N(c3cccc4ccccc34)c3ccc(-c4ccc(-c5ccccc5)cc4)c4c3oc3ccc5oc6ccccc6c5c34)cc21 ; c1ccc(-c2ccc(-c3ccc(N(c4ccccc4)c4ccc5c(c4)C(c4ccccc4)(c4ccccc4)c4ccccc4-5)c4oc5ccc6oc7ccccc7c6c5c34)cc2)cc1 ; c1ccc(-c2ccc(-c3ccc(N(c4ccccc4)c4ccc5c(c4)C4(c6ccccc6-c6ccccc64)c4ccccc4-5)c4oc5ccc6oc7ccccc7c6c5c34)cc2)cc1]

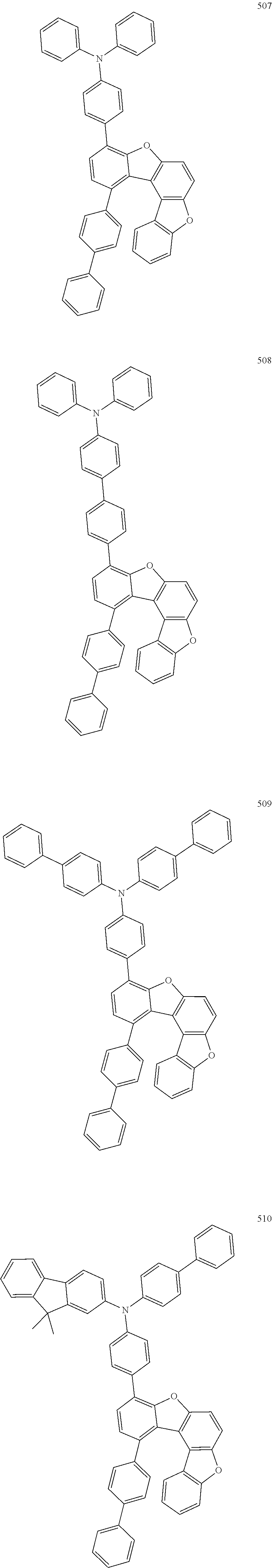 [SMILES: c1ccc(-c2ccc(-c3ccc(-c4ccc(N(c5ccccc5)c5ccccc5)cc4)c4oc5ccc6oc7ccccc7c6c5c34)cc2)cc1 ; c1ccc(-c2ccc(-c3ccc(-c4ccc(-c5ccc(N(c6ccccc6)c6ccccc6)cc5)cc4)c4oc5ccc6oc7ccccc7c6c5c34)cc2)cc1 ; c1ccc(-c2ccc(-c3ccc(-c4ccc(N(c5ccc(-c6ccccc6)cc5)c5ccc(-c6ccccc6)cc5)cc4)c4oc5ccc6oc7ccccc7c6c5c34)cc2)cc1 ; CC1(C)c2ccccc2-c2ccc(N(c3ccc(-c4ccccc4)cc3)c3ccc(-c4ccc(-c5ccc(-c6ccccc6)cc5)c5c4oc4ccc6oc7ccccc7c6c45)cc3)cc21]

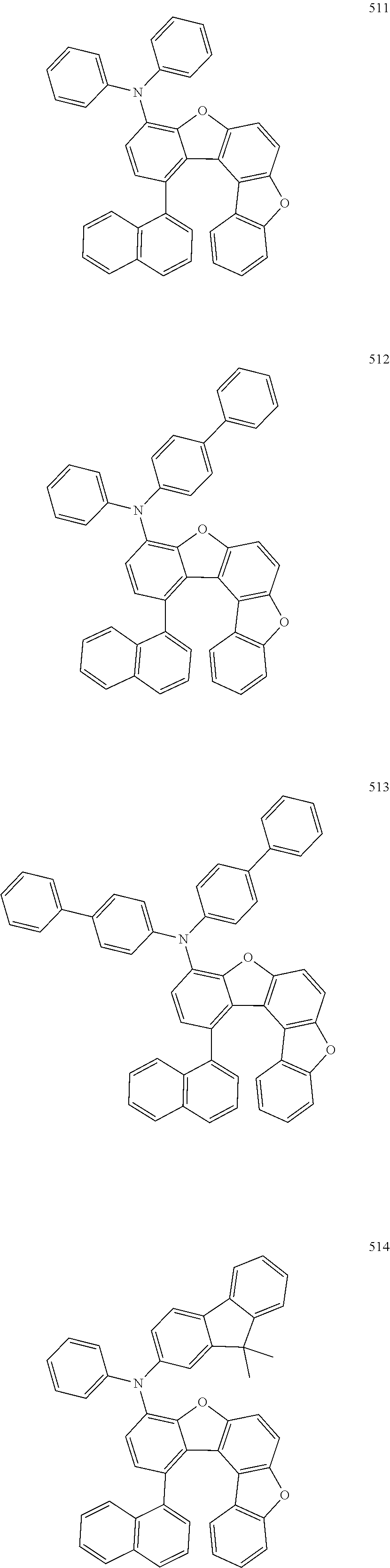 [SMILES: c1ccc(N(c2ccccc2)c2ccc(-c3cccc4ccccc34)c3c2oc2ccc4oc5ccccc5c4c23)cc1 ; c1ccc(-c2ccc(N(c3ccccc3)c3ccc(-c4cccc5ccccc45)c4c3oc3ccc5oc6ccccc6c5c34)cc2)cc1 ; c1ccc(-c2ccc(N(c3ccc(-c4ccccc4)cc3)c3ccc(-c4cccc5ccccc45)c4c3oc3ccc5oc6ccccc6c5c34)cc2)cc1 ; CC1(C)c2ccccc2-c2ccc(N(c3ccccc3)c3ccc(-c4cccc5ccccc45)c4c3oc3ccc5oc6ccccc6c5c34)cc21]

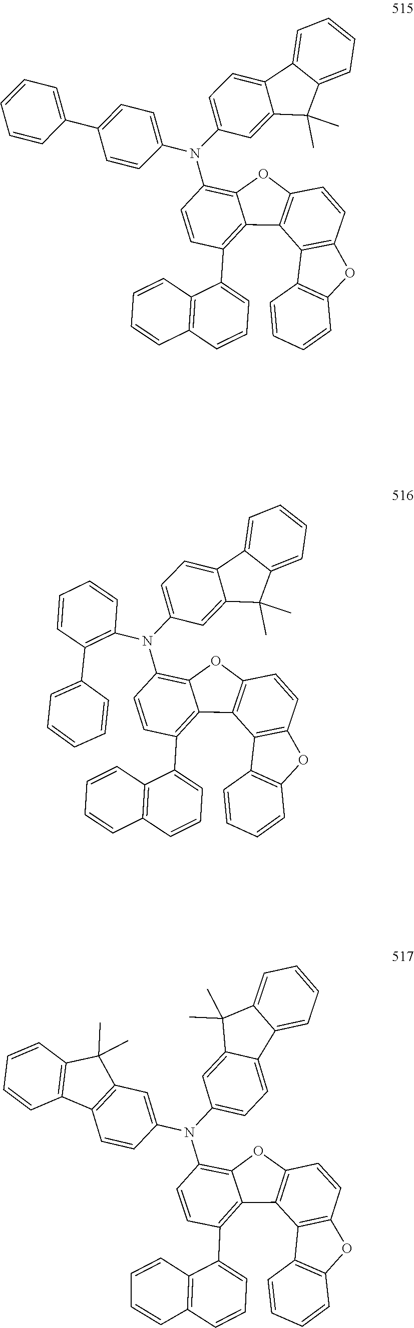 [SMILES: CC1(C)c2ccccc2-c2ccc(N(c3ccc(-c4ccccc4)cc3)c3ccc(-c4cccc5ccccc45)c4c3oc3ccc5oc6ccccc6c5c34)cc21 ; CC1(C)c2ccccc2-c2ccc(N(c3ccccc3-c3ccccc3)c3ccc(-c4cccc5ccccc45)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4cccc5ccccc45)c4c3oc3ccc5oc6ccccc6c5c34)cc21]

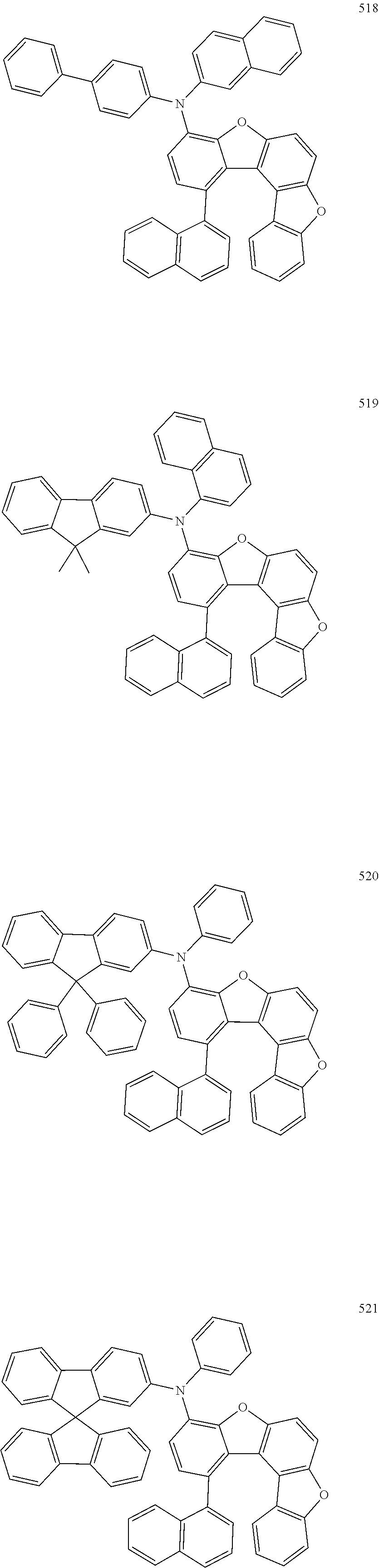 [SMILES: c1ccc(-c2ccc(N(c3ccc4ccccc4c3)c3ccc(-c4cccc5ccccc45)c4c3oc3ccc5oc6ccccc6c5c34)cc2)cc1 ; CC1(C)c2ccccc2-c2ccc(N(c3cccc4ccccc34)c3ccc(-c4cccc5ccccc45)c4c3oc3ccc5oc6ccccc6c5c34)cc21 ; c1ccc(N(c2ccc3c(c2)C(c2ccccc2)(c2ccccc2)c2ccccc2-3)c2ccc(-c3cccc4ccccc34)c3c2oc2ccc4oc5ccccc5c4c23)cc1 ; c1ccc(N(c2ccc3c(c2)C2(c4ccccc4-c4ccccc42)c2ccccc2-3)c2ccc(-c3cccc4ccccc34)c3c2oc2ccc4oc5ccccc5c4c23)cc1]

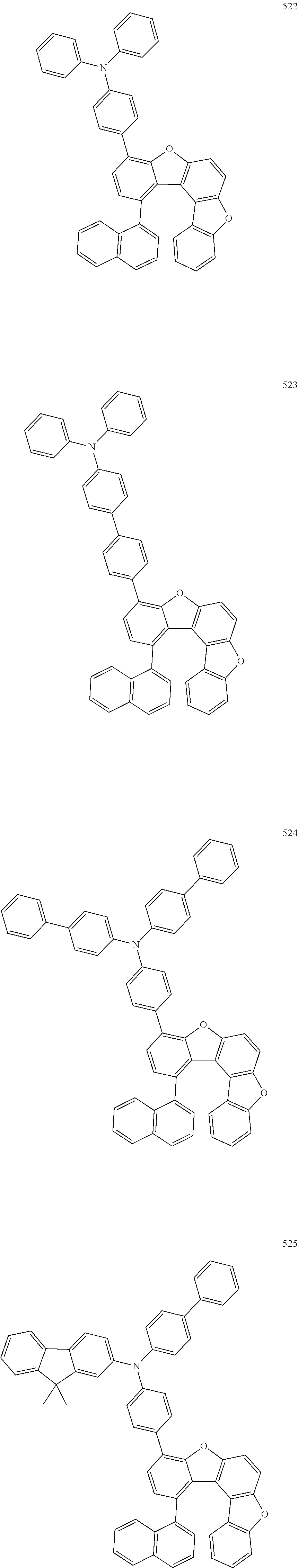 [SMILES: c1ccc(N(c2ccccc2)c2ccc(-c3ccc(-c4cccc5ccccc45)c4c3oc3ccc5oc6ccccc6c5c34)cc2)cc1 ; c1ccc(N(c2ccccc2)c2ccc(-c3ccc(-c4ccc(-c5cccc6ccccc56)c5c4oc4ccc6oc7ccccc7c6c45)cc3)cc2)cc1 ; c1ccc(-c2ccc(N(c3ccc(-c4ccccc4)cc3)c3ccc(-c4ccc(-c5cccc6ccccc56)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5cccc6ccccc56)c5c4oc4ccc6oc7ccccc7c6c45)cc3)cc21]

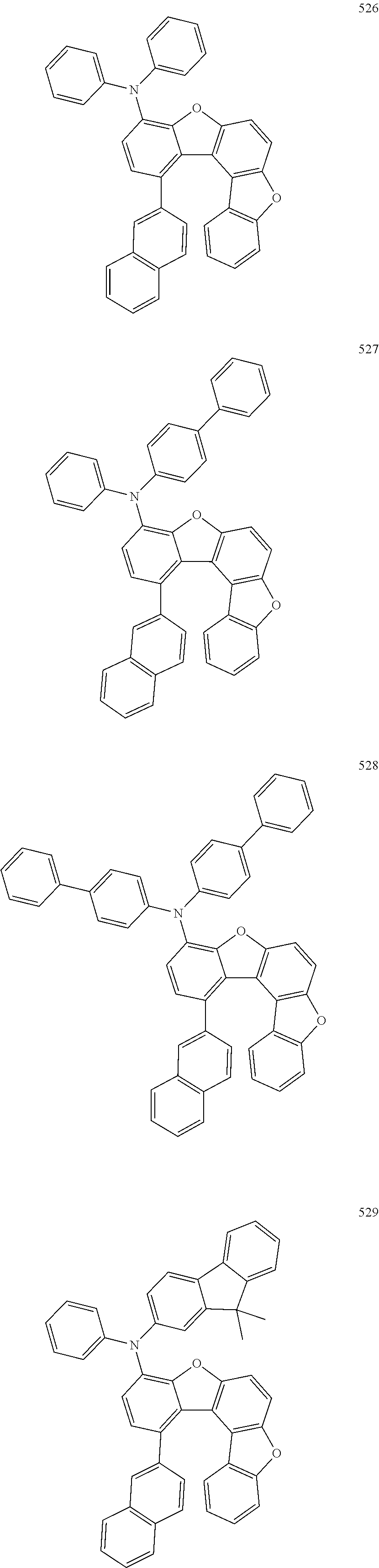 [SMILES: c1ccc(N(c2ccccc2)c2ccc(-c3ccc4ccccc4c3)c3c2oc2ccc4oc5ccccc5c4c23)cc1 ; c1ccc(-c2ccc(N(c3ccccc3)c3ccc(-c4ccc5ccccc5c4)c4c3oc3ccc5oc6ccccc6c5c34)cc2)cc1 ; c1ccc(-c2ccc(N(c3ccc(-c4ccccc4)cc3)c3ccc(-c4ccc5ccccc5c4)c4c3oc3ccc5oc6ccccc6c5c34)cc2)cc1 ; CC1(C)c2ccccc2-c2ccc(N(c3ccccc3)c3ccc(-c4ccc5ccccc5c4)c4c3oc3ccc5oc6ccccc6c5c34)cc21]

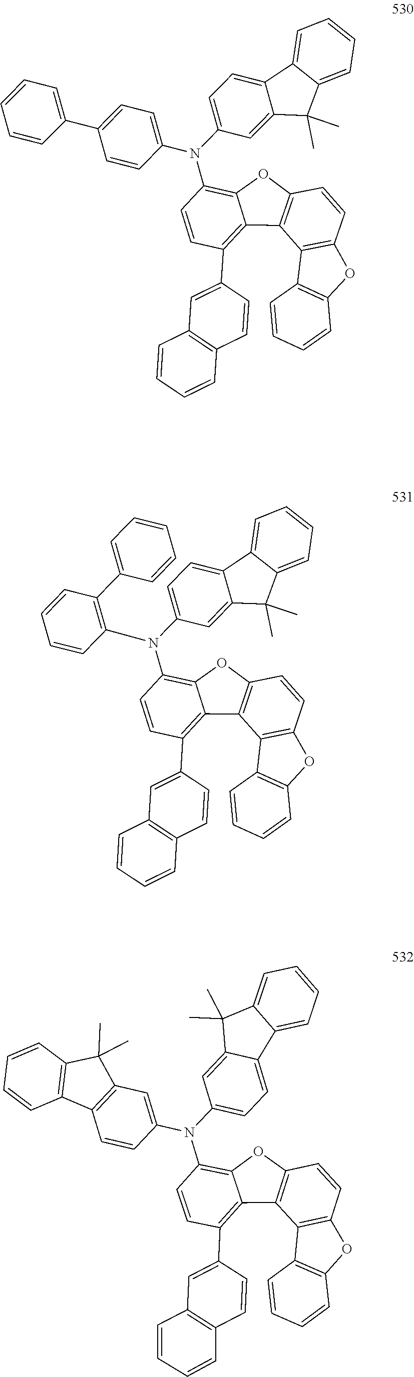 [SMILES: CC1(C)c2ccccc2-c2ccc(N(c3ccc(-c4ccccc4)cc3)c3ccc(-c4ccc5ccccc5c4)c4c3oc3ccc5oc6ccccc6c5c34)cc21 ; CC1(C)c2ccccc2-c2ccc(N(c3ccccc3-c3ccccc3)c3ccc(-c4ccc5ccccc5c4)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4ccc5ccccc5c4)c4c3oc3ccc5oc6ccccc6c5c34)cc21]

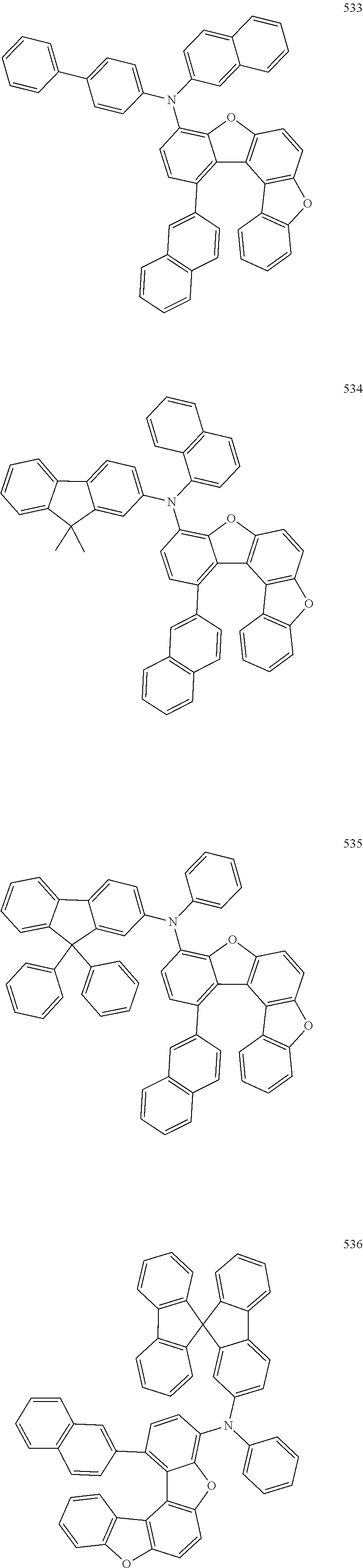 [SMILES: c1ccc(-c2ccc(N(c3ccc4ccccc4c3)c3ccc(-c4ccc5ccccc5c4)c4c3oc3ccc5oc6ccccc6c5c34)cc2)cc1 ; CC1(C)c2ccccc2-c2ccc(N(c3cccc4ccccc34)c3ccc(-c4ccc5ccccc5c4)c4c3oc3ccc5oc6ccccc6c5c34)cc21 ; c1ccc(N(c2ccc3c(c2)C(c2ccccc2)(c2ccccc2)c2ccccc2-3)c2ccc(-c3ccc4ccccc4c3)c3c2oc2ccc4oc5ccccc5c4c23)cc1 ; c1ccc(N(c2ccc3c(c2)C2(c4ccccc4-c4ccccc42)c2ccccc2-3)c2ccc(-c3ccc4ccccc4c3)c3c2oc2ccc4oc5ccccc5c4c23)cc1]

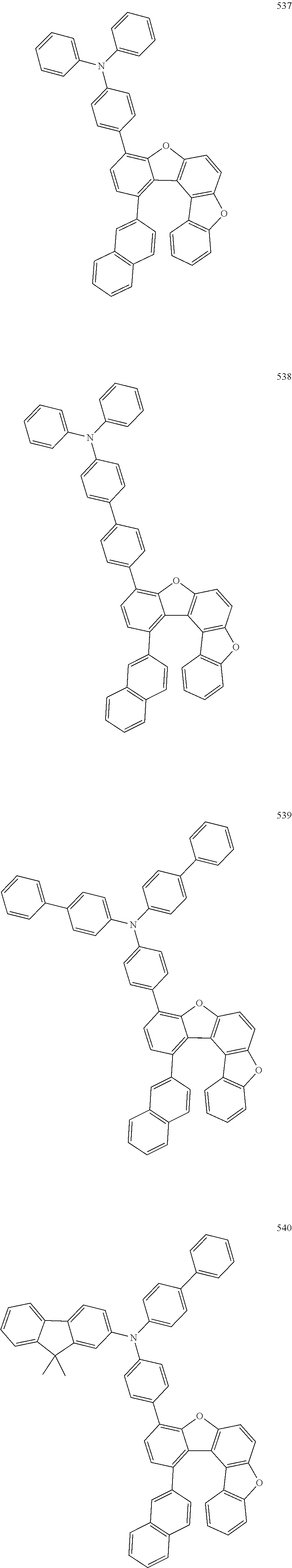 [SMILES: c1ccc(N(c2ccccc2)c2ccc(-c3ccc(-c4ccc5ccccc5c4)c4c3oc3ccc5oc6ccccc6c5c34)cc2)cc1 ; c1ccc(N(c2ccccc2)c2ccc(-c3ccc(-c4ccc(-c5ccc6ccccc6c5)c5c4oc4ccc6oc7ccccc7c6c45)cc3)cc2)cc1 ; c1ccc(-c2ccc(N(c3ccc(-c4ccccc4)cc3)c3ccc(-c4ccc(-c5ccc6ccccc6c5)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5ccc6ccccc6c5)c5c4oc4ccc6oc7ccccc7c6c45)cc3)cc21]

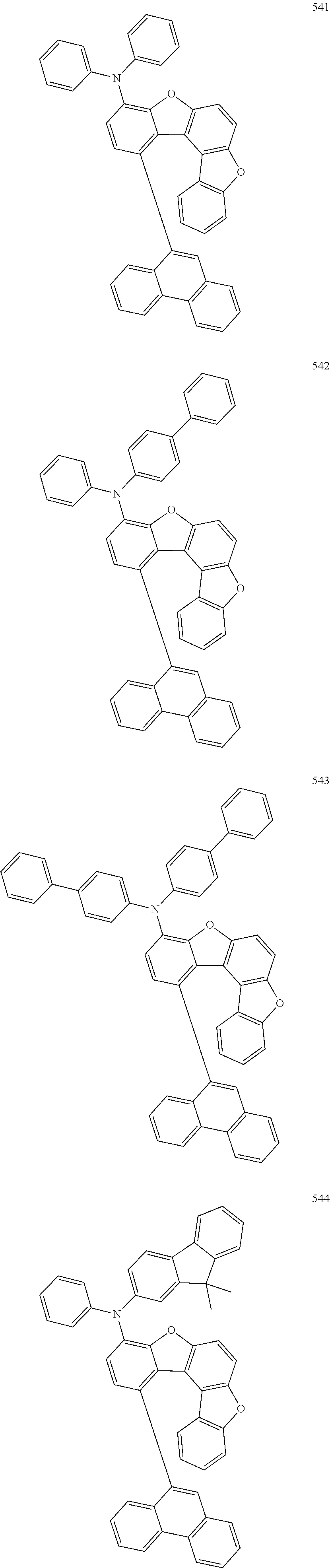 [SMILES: c1ccc(N(c2ccccc2)c2ccc(-c3cc4ccccc4c4ccccc34)c3c2oc2ccc4oc5ccccc5c4c23)cc1 ; c1ccc(-c2ccc(N(c3ccccc3)c3ccc(-c4cc5ccccc5c5ccccc45)c4c3oc3ccc5oc6ccccc6c5c34)cc2)cc1 ; c1ccc(-c2ccc(N(c3ccc(-c4ccccc4)cc3)c3ccc(-c4cc5ccccc5c5ccccc45)c4c3oc3ccc5oc6ccccc6c5c34)cc2)cc1 ; CC1(C)c2ccccc2-c2ccc(N(c3ccccc3)c3ccc(-c4cc5ccccc5c5ccccc45)c4c3oc3ccc5oc6ccccc6c5c34)cc21]

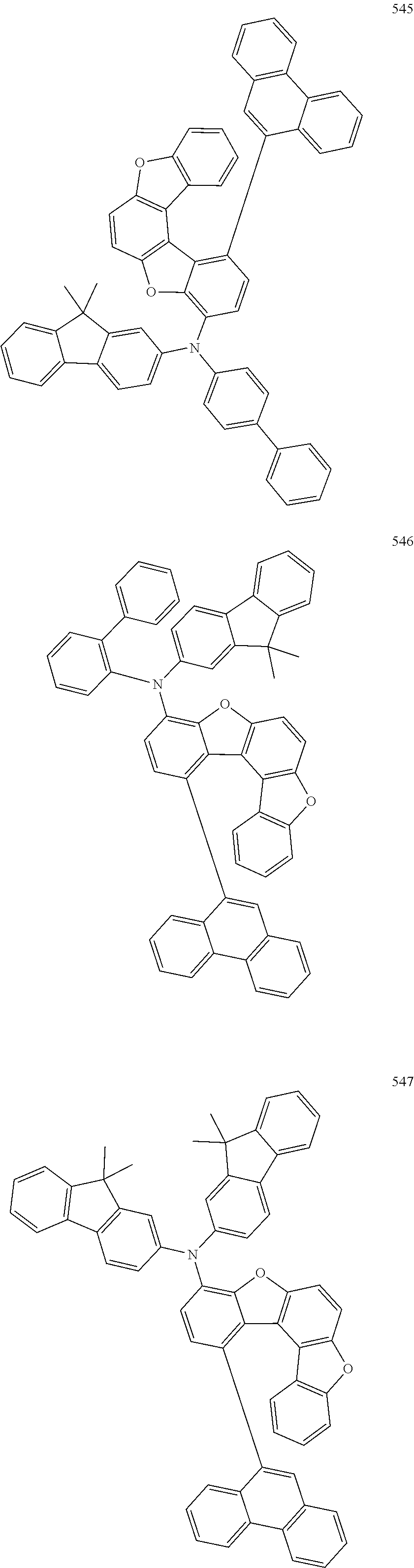

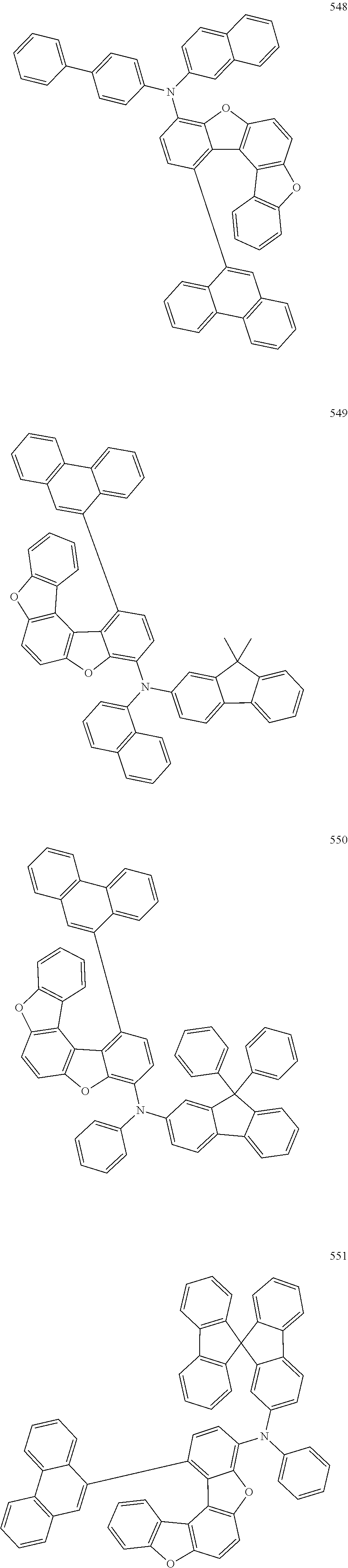 [SMILES: c1ccc(-c2ccc(N(c3ccc4ccccc4c3)c3ccc(-c4cc5ccccc5c5ccccc45)c4c3oc3ccc5oc6ccccc6c5c34)cc2)cc1 ; CC1(C)c2ccccc2-c2ccc(N(c3cccc4ccccc34)c3ccc(-c4cc5ccccc5c5ccccc45)c4c3oc3ccc5oc6ccccc6c5c34)cc21 ; c1ccc(N(c2ccc3c(c2)C(c2ccccc2)(c2ccccc2)c2ccccc2-3)c2ccc(-c3cc4ccccc4c4ccccc34)c3c2oc2ccc4oc5ccccc5c4c23)cc1 ; c1ccc(N(c2ccc3c(c2)C2(c4ccccc4-c4ccccc42)c2ccccc2-3)c2ccc(-c3cc4ccccc4c4ccccc34)c3c2oc2ccc4oc5ccccc5c4c23)cc1]

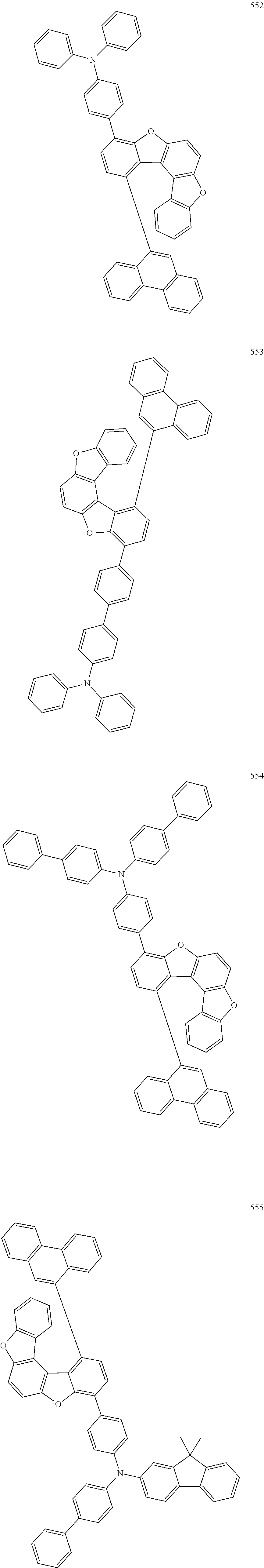 [SMILES: c1ccc(N(c2ccccc2)c2ccc(-c3ccc(-c4cc5ccccc5c5ccccc45)c4c3oc3ccc5oc6ccccc6c5c34)cc2)cc1 ; c1ccc(N(c2ccccc2)c2ccc(-c3ccc(-c4ccc(-c5cc6ccccc6c6ccccc56)c5c4oc4ccc6oc7ccccc7c6c45)cc3)cc2)cc1 ; c1ccc(-c2ccc(N(c3ccc(-c4ccccc4)cc3)c3ccc(-c4ccc(-c5cc6ccccc6c6ccccc56)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5cc6ccccc6c6ccccc56)c5c4oc4ccc6oc7ccccc7c6c45)cc3)cc21]

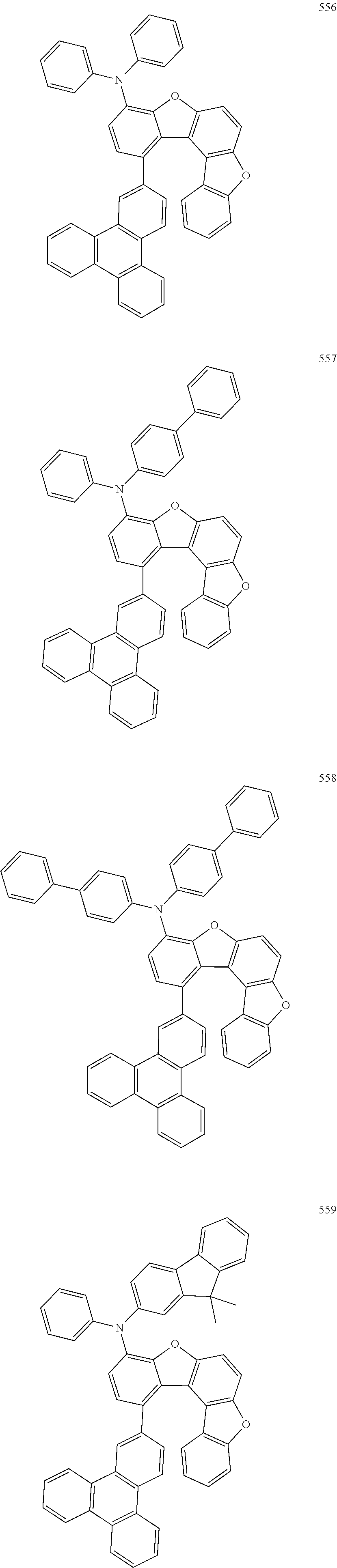 [SMILES: c1ccc(N(c2ccccc2)c2ccc(-c3ccc4c5ccccc5c5ccccc5c4c3)c3c2oc2ccc4oc5ccccc5c4c23)cc1 ; c1ccc(-c2ccc(N(c3ccccc3)c3ccc(-c4ccc5c6ccccc6c6ccccc6c5c4)c4c3oc3ccc5oc6ccccc6c5c34)cc2)cc1 ; c1ccc(-c2ccc(N(c3ccc(-c4ccccc4)cc3)c3ccc(-c4ccc5c6ccccc6c6ccccc6c5c4)c4c3oc3ccc5oc6ccccc6c5c34)cc2)cc1 ; CC1(C)c2ccccc2-c2ccc(N(c3ccccc3)c3ccc(-c4ccc5c6ccccc6c6ccccc6c5c4)c4c3oc3ccc5oc6ccccc6c5c34)cc21]

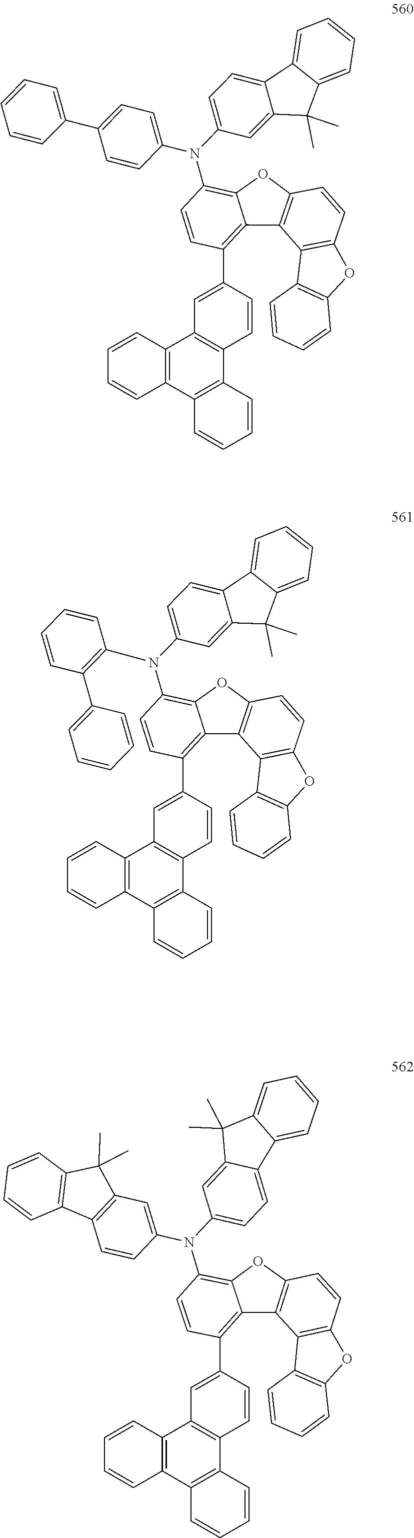 [SMILES: CC1(C)c2ccccc2-c2ccc(N(c3ccc(-c4ccccc4)cc3)c3ccc(-c4ccc5c6ccccc6c6ccccc6c5c4)c4c3oc3ccc5oc6ccccc6c5c34)cc21 ; CC1(C)c2ccccc2-c2ccc(N(c3ccccc3-c3ccccc3)c3ccc(-c4ccc5c6ccccc6c6ccccc6c5c4)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4ccc5c6ccccc6c6ccccc6c5c4)c4c3oc3ccc5oc6ccccc6c5c34)cc21]

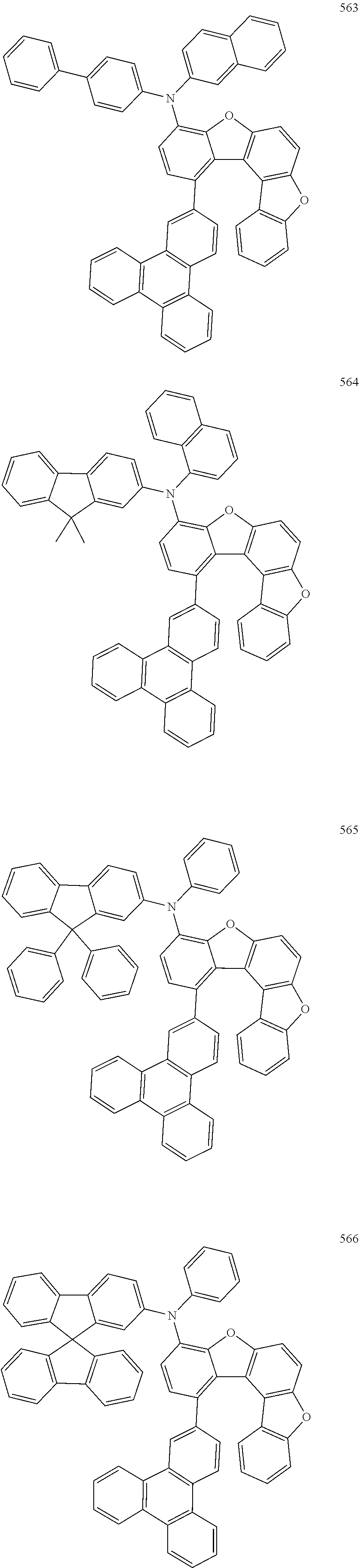 [SMILES: c1ccc(-c2ccc(N(c3ccc4ccccc4c3)c3ccc(-c4ccc5c6ccccc6c6ccccc6c5c4)c4c3oc3ccc5oc6ccccc6c5c34)cc2)cc1 ; CC1(C)c2ccccc2-c2ccc(N(c3cccc4ccccc34)c3ccc(-c4ccc5c6ccccc6c6ccccc6c5c4)c4c3oc3ccc5oc6ccccc6c5c34)cc21 ; c1ccc(N(c2ccc3c(c2)C(c2ccccc2)(c2ccccc2)c2ccccc2-3)c2ccc(-c3ccc4c5ccccc5c5ccccc5c4c3)c3c2oc2ccc4oc5ccccc5c4c23)cc1 ; c1ccc(N(c2ccc3c(c2)C2(c4ccccc4-c4ccccc42)c2ccccc2-3)c2ccc(-c3ccc4c5ccccc5c5ccccc5c4c3)c3c2oc2ccc4oc5ccccc5c4c23)cc1]

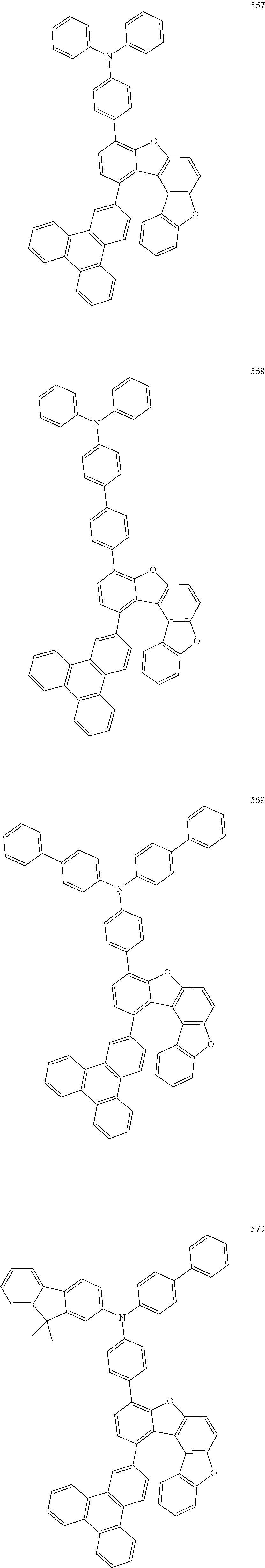 [SMILES: c1ccc(N(c2ccccc2)c2ccc(-c3ccc(-c4ccc5c6ccccc6c6ccccc6c5c4)c4c3oc3ccc5oc6ccccc6c5c34)cc2)cc1 ; c1ccc(N(c2ccccc2)c2ccc(-c3ccc(-c4ccc(-c5ccc6c7ccccc7c7ccccc7c6c5)c5c4oc4ccc6oc7ccccc7c6c45)cc3)cc2)cc1 ; c1ccc(-c2ccc(N(c3ccc(-c4ccccc4)cc3)c3ccc(-c4ccc(-c5ccc6c7ccccc7c7ccccc7c6c5)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5ccc6c7ccccc7c7ccccc7c6c5)c5c4oc4ccc6oc7ccccc7c6c45)cc3)cc21]

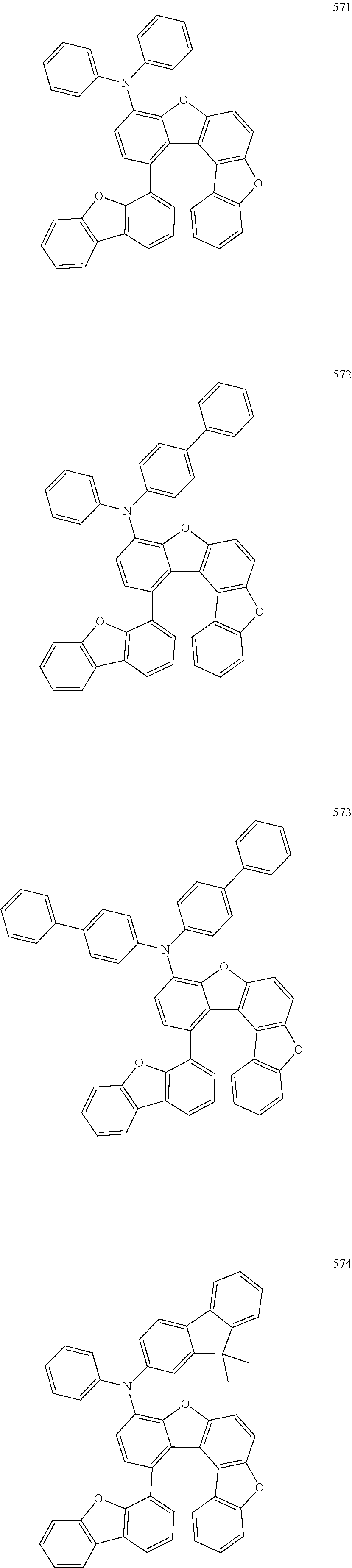 [SMILES: c1ccc(N(c2ccccc2)c2ccc(-c3cccc4c3oc3ccccc34)c3c2oc2ccc4oc5ccccc5c4c23)cc1 ; c1ccc(-c2ccc(N(c3ccccc3)c3ccc(-c4cccc5c4oc4ccccc45)c4c3oc3ccc5oc6ccccc6c5c34)cc2)cc1 ; c1ccc(-c2ccc(N(c3ccc(-c4ccccc4)cc3)c3ccc(-c4cccc5c4oc4ccccc45)c4c3oc3ccc5oc6ccccc6c5c34)cc2)cc1 ; CC1(C)c2ccccc2-c2ccc(N(c3ccccc3)c3ccc(-c4cccc5c4oc4ccccc45)c4c3oc3ccc5oc6ccccc6c5c34)cc21]

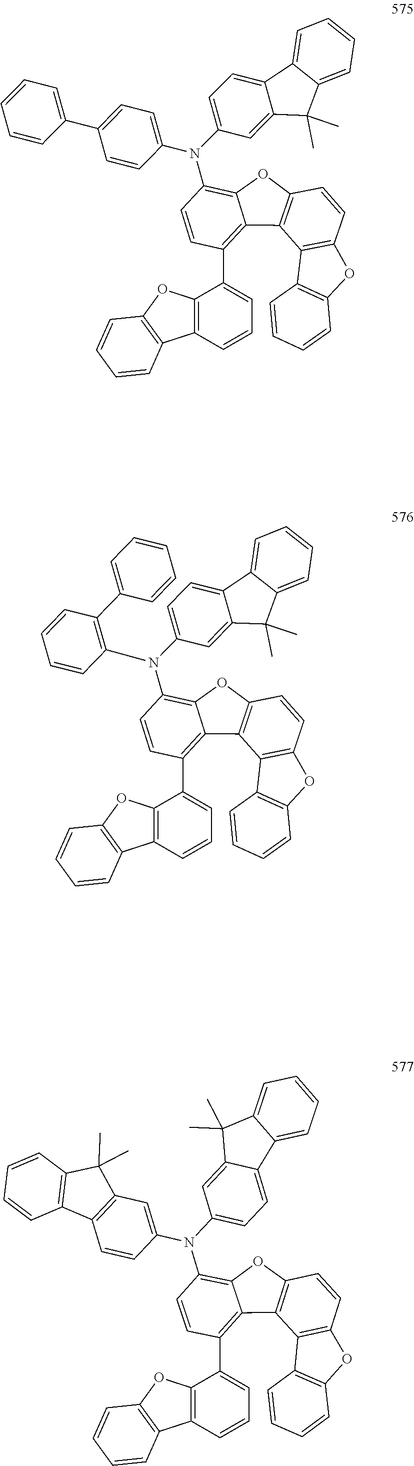 [SMILES: CC1(C)c2ccccc2-c2ccc(N(c3ccc(-c4ccccc4)cc3)c3ccc(-c4cccc5c4oc4ccccc45)c4c3oc3ccc5oc6ccccc6c5c34)cc21 ; CC1(C)c2ccccc2-c2ccc(N(c3ccccc3-c3ccccc3)c3ccc(-c4cccc5c4oc4ccccc45)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4cccc5c4oc4ccccc45)c4c3oc3ccc5oc6ccccc6c5c34)cc21]

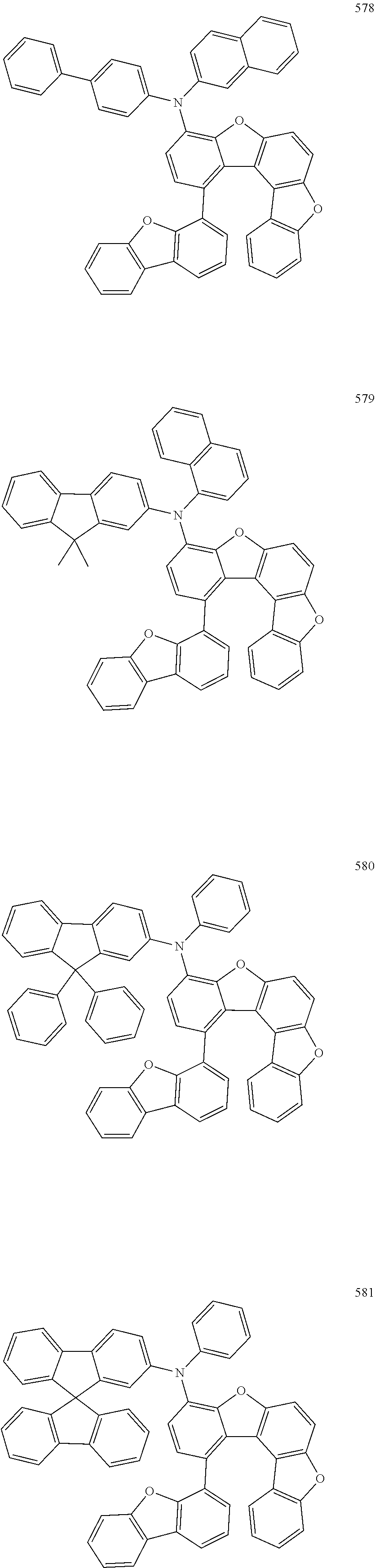 [SMILES: c1ccc(-c2ccc(N(c3ccc4ccccc4c3)c3ccc(-c4cccc5c4oc4ccccc45)c4c3oc3ccc5oc6ccccc6c5c34)cc2)cc1 ; CC1(C)c2ccccc2-c2ccc(N(c3cccc4ccccc34)c3ccc(-c4cccc5c4oc4ccccc45)c4c3oc3ccc5oc6ccccc6c5c34)cc21 ; c1ccc(N(c2ccc3c(c2)C(c2ccccc2)(c2ccccc2)c2ccccc2-3)c2ccc(-c3cccc4c3oc3ccccc34)c3c2oc2ccc4oc5ccccc5c4c23)cc1 ; c1ccc(N(c2ccc3c(c2)C2(c4ccccc4-c4ccccc42)c2ccccc2-3)c2ccc(-c3cccc4c3oc3ccccc34)c3c2oc2ccc4oc5ccccc5c4c23)cc1]

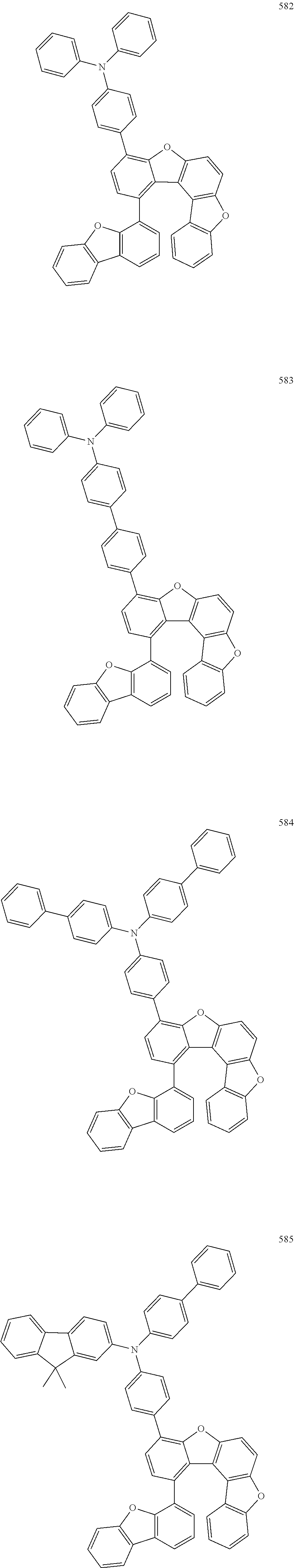 [SMILES: c1ccc(N(c2ccccc2)c2ccc(-c3ccc(-c4cccc5c4oc4ccccc45)c4c3oc3ccc5oc6ccccc6c5c34)cc2)cc1 ; c1ccc(N(c2ccccc2)c2ccc(-c3ccc(-c4ccc(-c5cccc6c5oc5ccccc56)c5c4oc4ccc6oc7ccccc7c6c45)cc3)cc2)cc1 ; c1ccc(-c2ccc(N(c3ccc(-c4ccccc4)cc3)c3ccc(-c4ccc(-c5cccc6c5oc5ccccc56)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5cccc6c5oc5ccccc56)c5c4oc4ccc6oc7ccccc7c6c45)cc3)cc21]

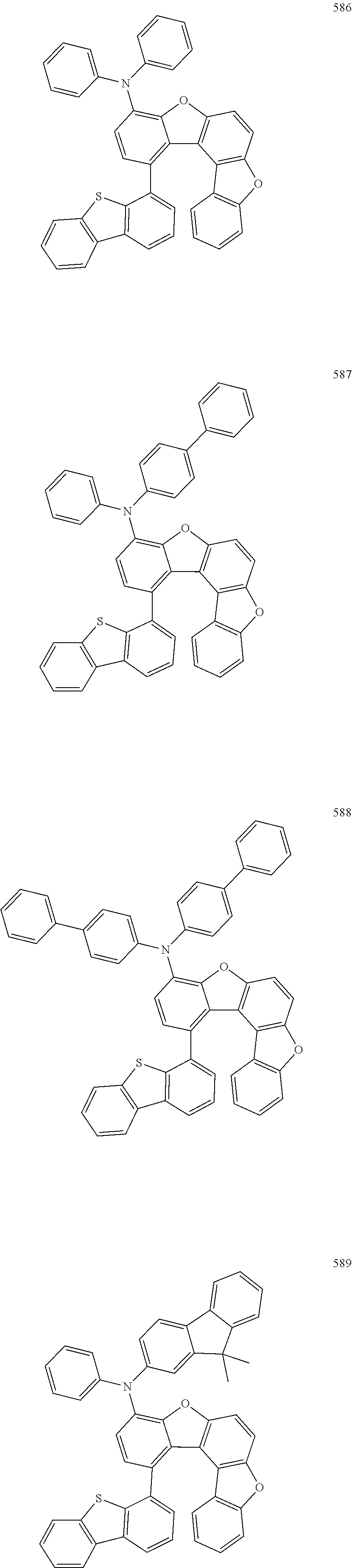 [SMILES: c1ccc(N(c2ccccc2)c2ccc(-c3cccc4c3sc3ccccc34)c3c2oc2ccc4oc5ccccc5c4c23)cc1 ; c1ccc(-c2ccc(N(c3ccccc3)c3ccc(-c4cccc5c4sc4ccccc45)c4c3oc3ccc5oc6ccccc6c5c34)cc2)cc1 ; c1ccc(-c2ccc(N(c3ccc(-c4ccccc4)cc3)c3ccc(-c4cccc5c4sc4ccccc45)c4c3oc3ccc5oc6ccccc6c5c34)cc2)cc1 ; CC1(C)c2ccccc2-c2ccc(N(c3ccccc3)c3ccc(-c4cccc5c4sc4ccccc45)c4c3oc3ccc5oc6ccccc6c5c34)cc21]

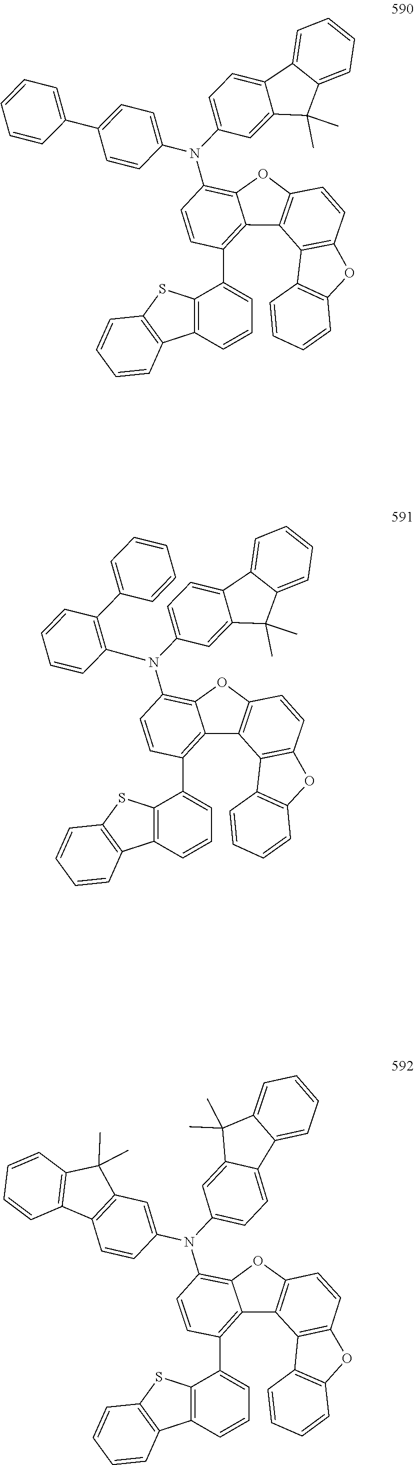 [SMILES: CC1(C)c2ccccc2-c2ccc(N(c3ccc(-c4ccccc4)cc3)c3ccc(-c4cccc5c4sc4ccccc45)c4c3oc3ccc5oc6ccccc6c5c34)cc21 ; CC1(C)c2ccccc2-c2ccc(N(c3ccccc3-c3ccccc3)c3ccc(-c4cccc5c4sc4ccccc45)c4c3oc3ccc5oc6ccccc6c5c34)cc21 ; CC1(C)c2ccccc2-c2ccc(N(c3ccc4c(c3)C(C)(C)c3ccccc3-4)c3ccc(-c4cccc5c4sc4ccccc45)c4c3oc3ccc5oc6ccccc6c5c34)cc21]

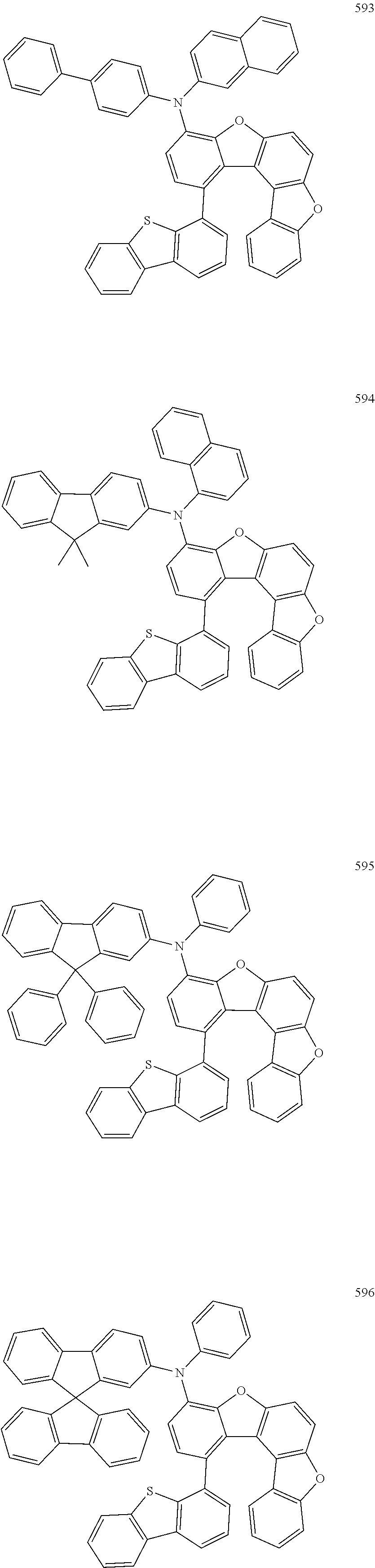 [SMILES: c1ccc(-c2ccc(N(c3ccc4ccccc4c3)c3ccc(-c4cccc5c4sc4ccccc45)c4c3oc3ccc5oc6ccccc6c5c34)cc2)cc1 ; CC1(C)c2ccccc2-c2ccc(N(c3cccc4ccccc34)c3ccc(-c4cccc5c4sc4ccccc45)c4c3oc3ccc5oc6ccccc6c5c34)cc21 ; c1ccc(N(c2ccc3c(c2)C(c2ccccc2)(c2ccccc2)c2ccccc2-3)c2ccc(-c3cccc4c3sc3ccccc34)c3c2oc2ccc4oc5ccccc5c4c23)cc1 ; c1ccc(N(c2ccc3c(c2)C2(c4ccccc4-c4ccccc42)c2ccccc2-3)c2ccc(-c3cccc4c3sc3ccccc34)c3c2oc2ccc4oc5ccccc5c4c23)cc1]

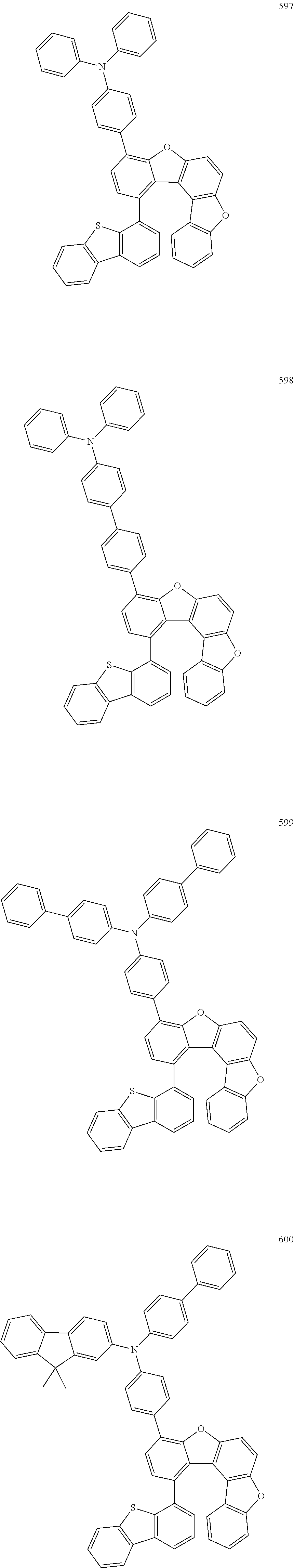 [SMILES: c1ccc(N(c2ccccc2)c2ccc(-c3ccc(-c4cccc5c4sc4ccccc45)c4c3oc3ccc5oc6ccccc6c5c34)cc2)cc1 ; c1ccc(N(c2ccccc2)c2ccc(-c3ccc(-c4ccc(-c5cccc6c5sc5ccccc56)c5c4oc4ccc6oc7ccccc7c6c45)cc3)cc2)cc1 ; c1ccc(-c2ccc(N(c3ccc(-c4ccccc4)cc3)c3ccc(-c4ccc(-c5cccc6c5sc5ccccc56)c5c4oc4ccc6oc7ccccc7c6c45)cc3)cc2)cc1 ; CC1(C)c2ccccc2-c2ccc(N(c3ccc(-c4ccccc4)cc3)c3ccc(-c4ccc(-c5cccc6c5sc5ccccc56)c5c4oc4ccc6oc7ccccc7c6c45)cc3)cc21]

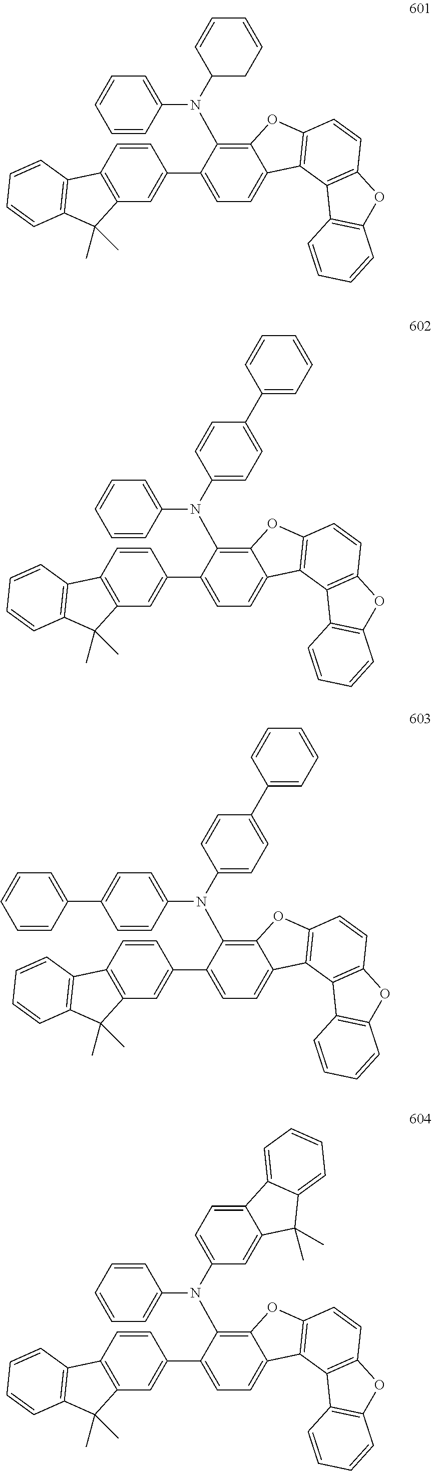 [SMILES: CC1(C)c2ccccc2-c2ccc(-c3ccc4c(oc5ccc6oc7ccccc7c6c54)c3N(c3ccccc3)C3C=CC=CC3)cc21 ; CC1(C)c2ccccc2-c2ccc(-c3ccc4c(oc5ccc6oc7ccccc7c6c54)c3N(c3ccccc3)c3ccc(-c4ccccc4)cc3)cc21 ; CC1(C)c2ccccc2-c2ccc(-c3ccc4c(oc5ccc6oc7ccccc7c6c54)c3N(c3ccc(-c4ccccc4)cc3)c3ccc(-c4ccccc4)cc3)cc21 ; CC1(C)c2ccccc2-c2ccc(-c3ccc4c(oc5ccc6oc7ccccc7c6c54)c3N(c3ccccc3)c3ccc4c(c3)C(C)(C)c3ccccc3-4)cc21]

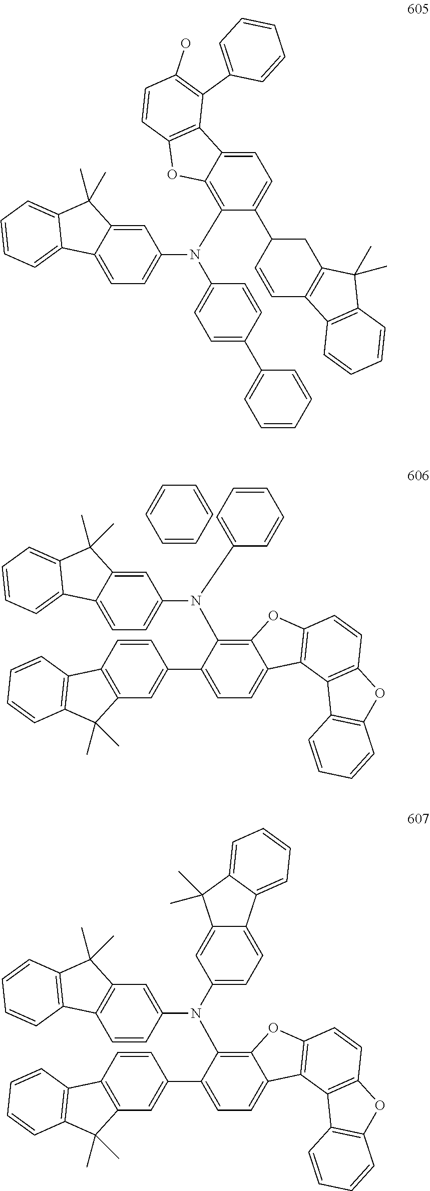

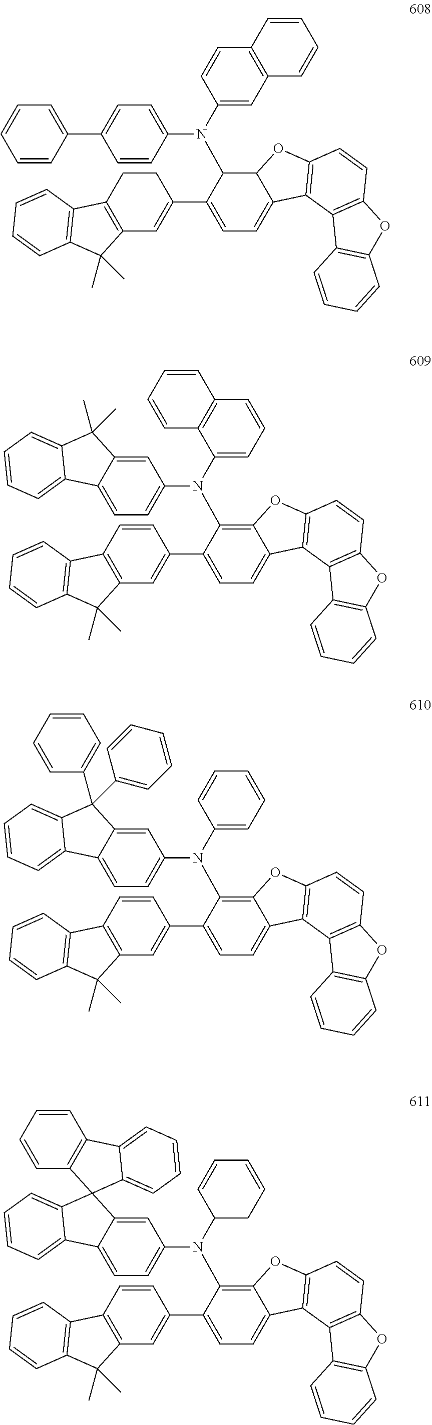 [SMILES: CC1(C)C2=C(CCC(C3=CC=C4c5c(ccc6oc7ccccc7c56)OC4C3N(c3ccc(-c4ccccc4)cc3)c3ccc4ccccc4c3)=C2)c2ccccc21 ; CC1(C)c2ccccc2-c2ccc(-c3ccc4c(oc5ccc6oc7ccccc7c6c54)c3N(c3ccc4c(c3)C(C)(C)c3ccccc3-4)c3cccc4ccccc34)cc21 ; CC1(C)c2ccccc2-c2ccc(-c3ccc4c(oc5ccc6oc7ccccc7c6c54)c3N(c3ccccc3)c3ccc4c(c3)C(c3ccccc3)(c3ccccc3)c3ccccc3-4)cc21 ; CC1(C)c2ccccc2-c2ccc(-c3ccc4c(oc5ccc6oc7ccccc7c6c54)c3N(c3ccc4c(c3)C3(c5ccccc5-c5ccccc53)c3ccccc3-4)C3C=CC=CC3)cc21]

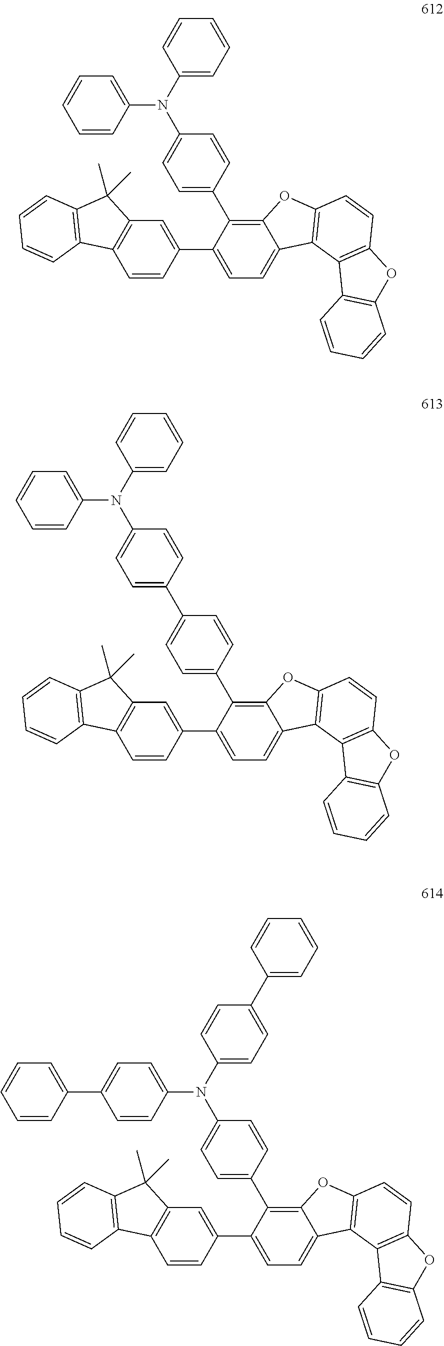 [SMILES: CC1(C)c2ccccc2-c2ccc(-c3ccc4c(oc5ccc6oc7ccccc7c6c54)c3-c3ccc(N(c4ccccc4)c4ccccc4)cc3)cc21 ; CC1(C)c2ccccc2-c2ccc(-c3ccc4c(oc5ccc6oc7ccccc7c6c54)c3-c3ccc(-c4ccc(N(c5ccccc5)c5ccccc5)cc4)cc3)cc21 ; CC1(C)c2ccccc2-c2ccc(-c3ccc4c(oc5ccc6oc7ccccc7c6c54)c3-c3ccc(N(c4ccc(-c5ccccc5)cc4)c4ccc(-c5ccccc5)cc4)cc3)cc21]

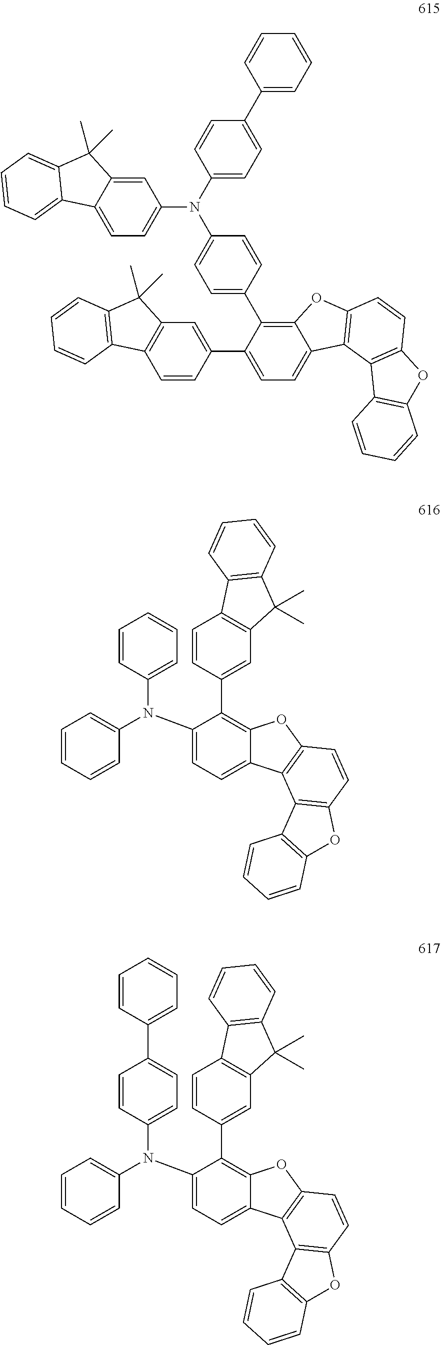 [SMILES: CC1(C)c2ccccc2-c2ccc(-c3ccc4c(oc5ccc6oc7ccccc7c6c54)c3-c3ccc(N(c4ccc(-c5ccccc5)cc4)c4ccc5c(c4)C(C)(C)c4ccccc4-5)cc3)cc21 ; CC1(C)c2ccccc2-c2ccc(-c3c(N(c4ccccc4)c4ccccc4)ccc4c3oc3ccc5oc6ccccc6c5c34)cc21 ; CC1(C)c2ccccc2-c2ccc(-c3c(N(c4ccccc4)c4ccc(-c5ccccc5)cc4)ccc4c3oc3ccc5oc6ccccc6c5c34)cc21]

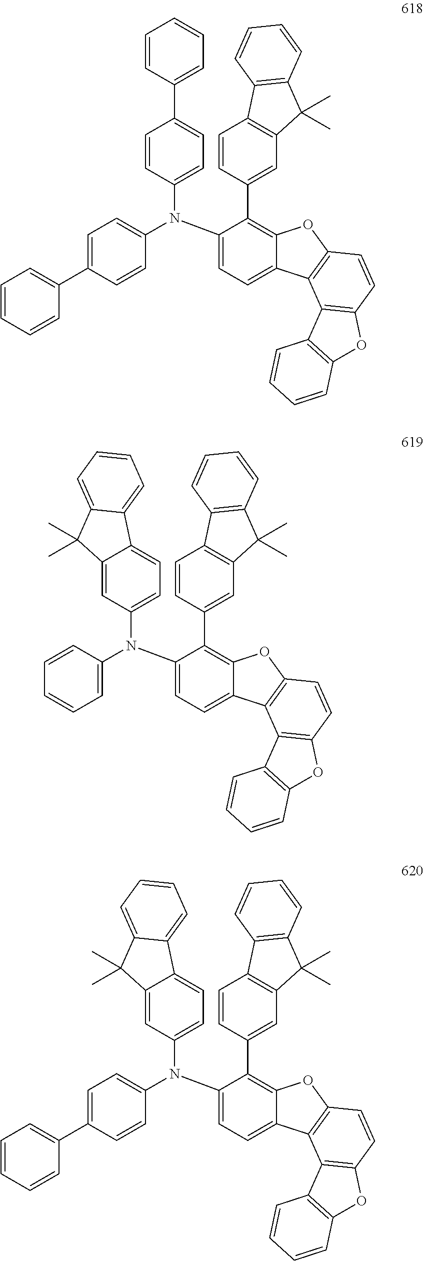 [SMILES: CC1(C)c2ccccc2-c2ccc(-c3c(N(c4ccc(-c5ccccc5)cc4)c4ccc(-c5ccccc5)cc4)ccc4c3oc3ccc5oc6ccccc6c5c34)cc21 ; CC1(C)c2ccccc2-c2ccc(-c3c(N(c4ccccc4)c4ccc5c(c4)C(C)(C)c4ccccc4-5)ccc4c3oc3ccc5oc6ccccc6c5c34)cc21 ; CC1(C)c2ccccc2-c2ccc(-c3c(N(c4ccc(-c5ccccc5)cc4)c4ccc5c(c4)C(C)(C)c4ccccc4-5)ccc4c3oc3ccc5oc6ccccc6c5c34)cc21]

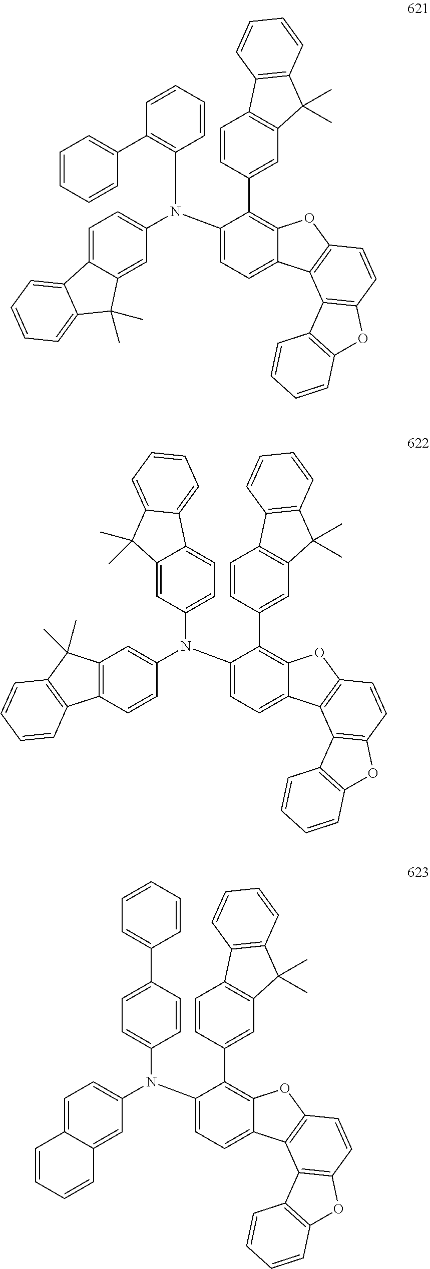 [SMILES: CC1(C)c2ccccc2-c2ccc(-c3c(N(c4ccc5c(c4)C(C)(C)c4ccccc4-5)c4ccccc4-c4ccccc4)ccc4c3oc3ccc5oc6ccccc6c5c34)cc21 ; CC1(C)c2ccccc2-c2ccc(-c3c(N(c4ccc5c(c4)C(C)(C)c4ccccc4-5)c4ccc5c(c4)C(C)(C)c4ccccc4-5)ccc4c3oc3ccc5oc6ccccc6c5c34)cc21 ; CC1(C)c2ccccc2-c2ccc(-c3c(N(c4ccc(-c5ccccc5)cc4)c4ccc5ccccc5c4)ccc4c3oc3ccc5oc6ccccc6c5c34)cc21]

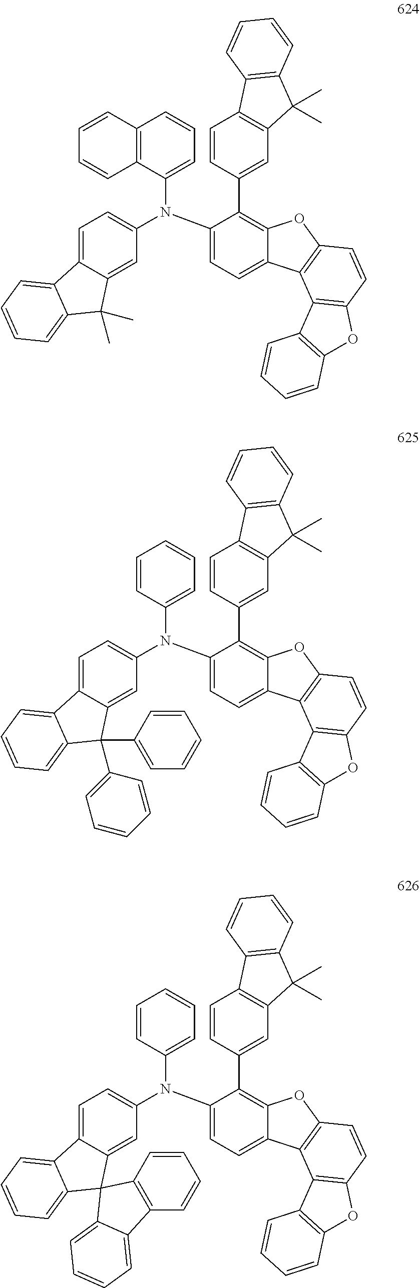 [SMILES: CC1(C)c2ccccc2-c2ccc(-c3c(N(c4ccc5c(c4)C(C)(C)c4ccccc4-5)c4cccc5ccccc45)ccc4c3oc3ccc5oc6ccccc6c5c34)cc21 ; CC1(C)c2ccccc2-c2ccc(-c3c(N(c4ccccc4)c4ccc5c(c4)C(c4ccccc4)(c4ccccc4)c4ccccc4-5)ccc4c3oc3ccc5oc6ccccc6c5c34)cc21 ; CC1(C)c2ccccc2-c2ccc(-c3c(N(c4ccccc4)c4ccc5c(c4)C4(c6ccccc6-c6ccccc64)c4ccccc4-5)ccc4c3oc3ccc5oc6ccccc6c5c34)cc21]

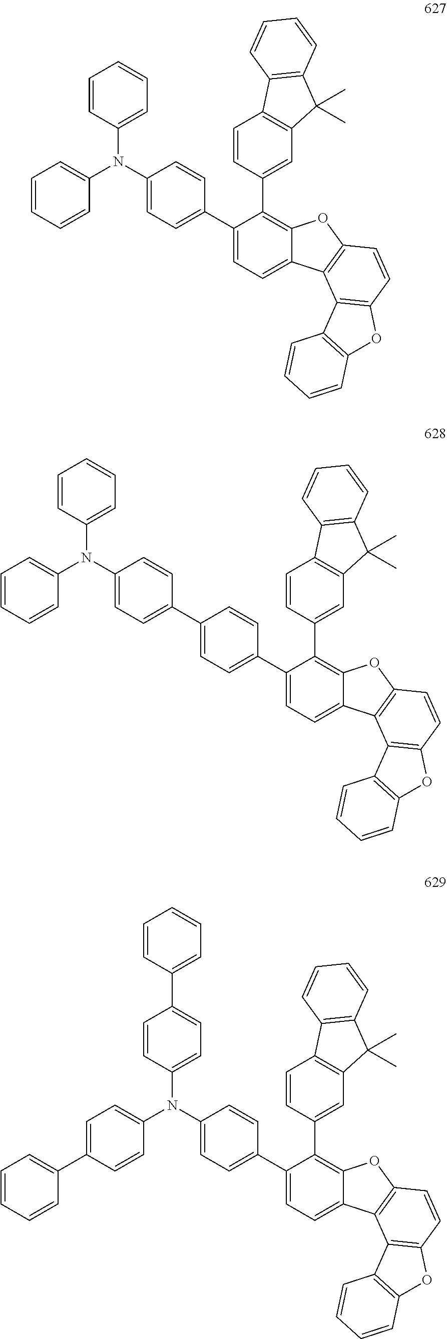 [SMILES: CC1(C)c2ccccc2-c2ccc(-c3c(-c4ccc(N(c5ccccc5)c5ccccc5)cc4)ccc4c3oc3ccc5oc6ccccc6c5c34)cc21 ; CC1(C)c2ccccc2-c2ccc(-c3c(-c4ccc(-c5ccc(N(c6ccccc6)c6ccccc6)cc5)cc4)ccc4c3oc3ccc5oc6ccccc6c5c34)cc21 ; CC1(C)c2ccccc2-c2ccc(-c3c(-c4ccc(N(c5ccc(-c6ccccc6)cc5)c5ccc(-c6ccccc6)cc5)cc4)ccc4c3oc3ccc5oc6ccccc6c5c34)cc21]

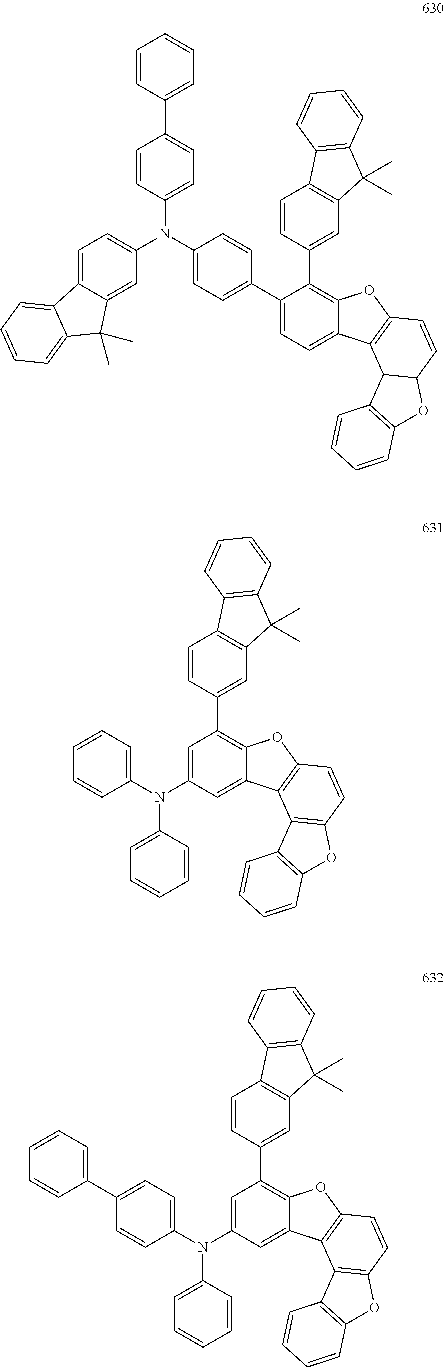 [SMILES: CC1(C)c2ccccc2-c2ccc(-c3c(-c4ccc(N(c5ccc(-c6ccccc6)cc5)c5ccc6c(c5)C(C)(C)c5ccccc5-6)cc4)ccc4c5c(oc34)C=CC3Oc4ccccc4C53)cc21 ; CC1(C)c2ccccc2-c2ccc(-c3cc(N(c4ccccc4)c4ccccc4)cc4c3oc3ccc5oc6ccccc6c5c34)cc21 ; CC1(C)c2ccccc2-c2ccc(-c3cc(N(c4ccccc4)c4ccc(-c5ccccc5)cc4)cc4c3oc3ccc5oc6ccccc6c5c34)cc21]

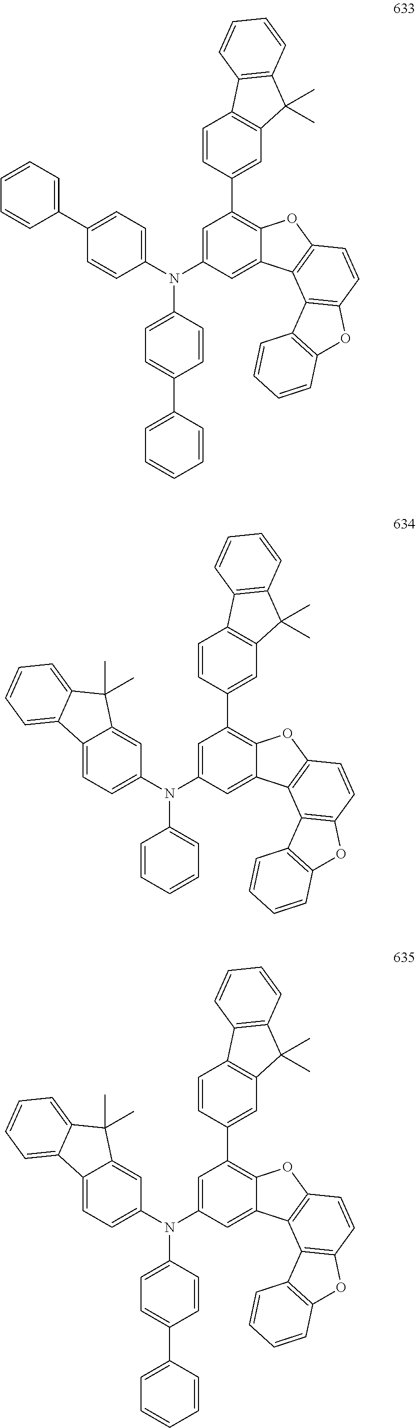 [SMILES: CC1(C)c2ccccc2-c2ccc(-c3cc(N(c4ccc(-c5ccccc5)cc4)c4ccc(-c5ccccc5)cc4)cc4c3oc3ccc5oc6ccccc6c5c34)cc21 ; CC1(C)c2ccccc2-c2ccc(-c3cc(N(c4ccccc4)c4ccc5c(c4)C(C)(C)c4ccccc4-5)cc4c3oc3ccc5oc6ccccc6c5c34)cc21 ; CC1(C)c2ccccc2-c2ccc(-c3cc(N(c4ccc(-c5ccccc5)cc4)c4ccc5c(c4)C(C)(C)c4ccccc4-5)cc4c3oc3ccc5oc6ccccc6c5c34)cc21]

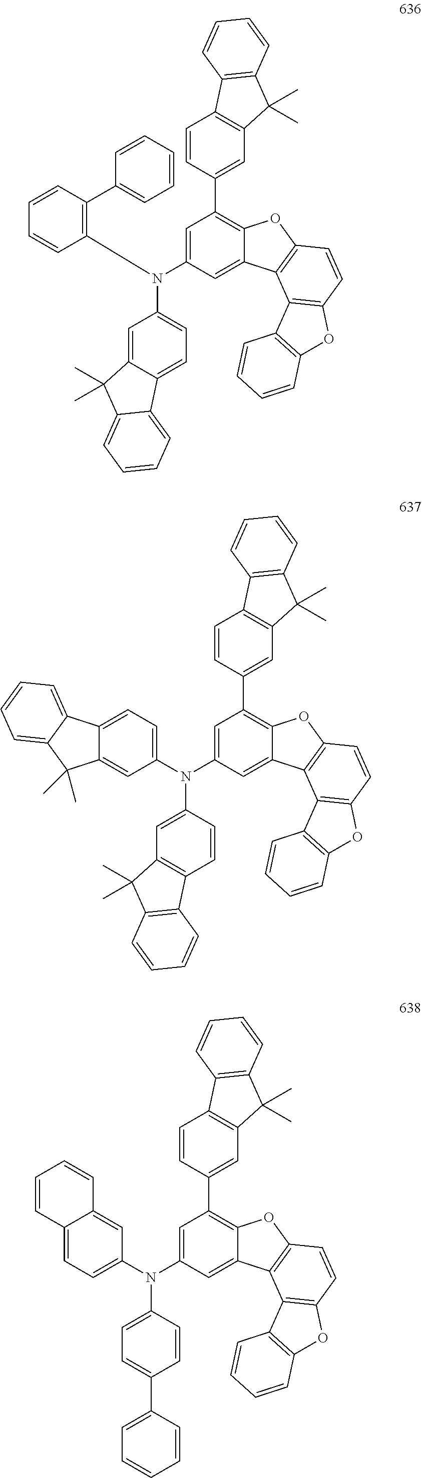 [SMILES: CC1(C)c2ccccc2-c2ccc(-c3cc(N(c4ccc5c(c4)C(C)(C)c4ccccc4-5)c4ccccc4-c4ccccc4)cc4c3oc3ccc5oc6ccccc6c5c34)cc21 ; CC1(C)c2ccccc2-c2ccc(-c3cc(N(c4ccc5c(c4)C(C)(C)c4ccccc4-5)c4ccc5c(c4)C(C)(C)c4ccccc4-5)cc4c3oc3ccc5oc6ccccc6c5c34)cc21 ; CC1(C)c2ccccc2-c2ccc(-c3cc(N(c4ccc(-c5ccccc5)cc4)c4ccc5ccccc5c4)cc4c3oc3ccc5oc6ccccc6c5c34)cc21]

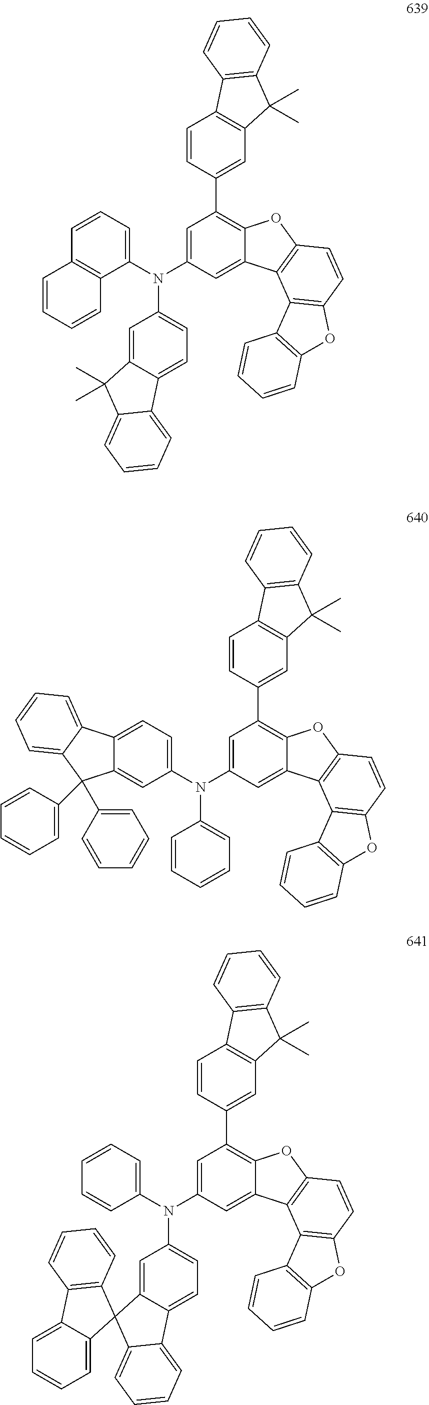 [SMILES: CC1(C)c2ccccc2-c2ccc(-c3cc(N(c4ccc5c(c4)C(C)(C)c4ccccc4-5)c4cccc5ccccc45)cc4c3oc3ccc5oc6ccccc6c5c34)cc21 ; CC1(C)c2ccccc2-c2ccc(-c3cc(N(c4ccccc4)c4ccc5c(c4)C(c4ccccc4)(c4ccccc4)c4ccccc4-5)cc4c3oc3ccc5oc6ccccc6c5c34)cc21 ; CC1(C)c2ccccc2-c2ccc(-c3cc(N(c4ccccc4)c4ccc5c(c4)C4(c6ccccc6-c6ccccc64)c4ccccc4-5)cc4c3oc3ccc5oc6ccccc6c5c34)cc21]

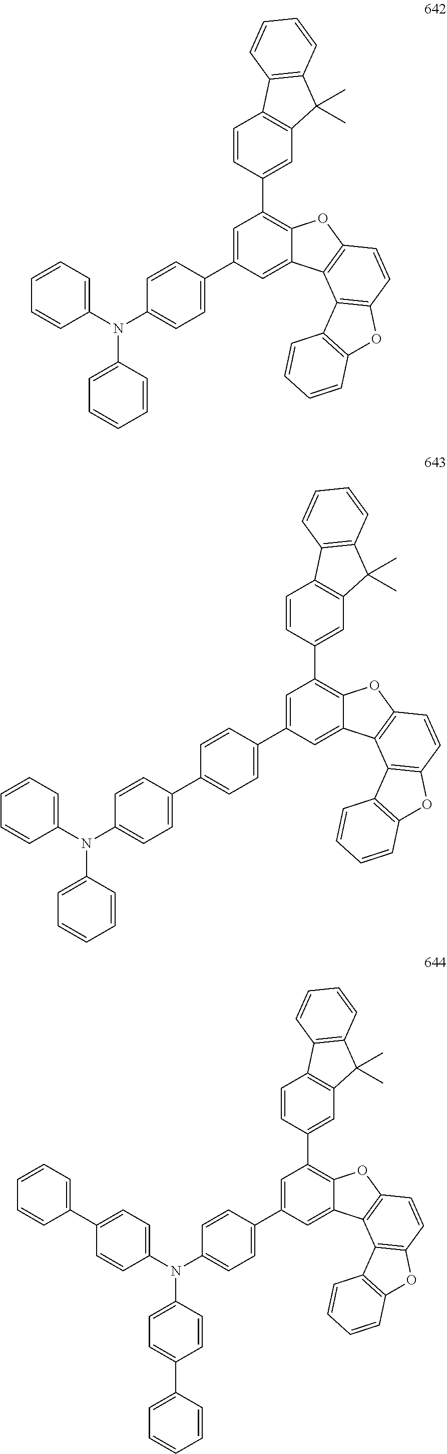 [SMILES: CC1(C)c2ccccc2-c2ccc(-c3cc(-c4ccc(N(c5ccccc5)c5ccccc5)cc4)cc4c3oc3ccc5oc6ccccc6c5c34)cc21 ; CC1(C)c2ccccc2-c2ccc(-c3cc(-c4ccc(-c5ccc(N(c6ccccc6)c6ccccc6)cc5)cc4)cc4c3oc3ccc5oc6ccccc6c5c34)cc21 ; CC1(C)c2ccccc2-c2ccc(-c3cc(-c4ccc(N(c5ccc(-c6ccccc6)cc5)c5ccc(-c6ccccc6)cc5)cc4)cc4c3oc3ccc5oc6ccccc6c5c34)cc21]

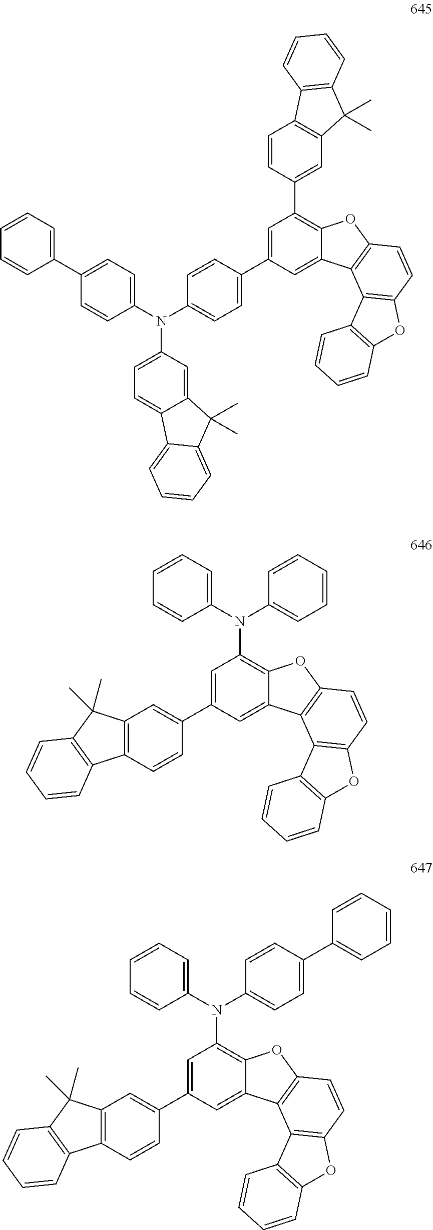 [SMILES: CC1(C)c2ccccc2-c2ccc(-c3cc(-c4ccc(N(c5ccc(-c6ccccc6)cc5)c5ccc6c(c5)C(C)(C)c5ccccc5-6)cc4)cc4c3oc3ccc5oc6ccccc6c5c34)cc21 ; CC1(C)c2ccccc2-c2ccc(-c3cc(N(c4ccccc4)c4ccccc4)c4oc5ccc6oc7ccccc7c6c5c4c3)cc21 ; CC1(C)c2ccccc2-c2ccc(-c3cc(N(c4ccccc4)c4ccc(-c5ccccc5)cc4)c4oc5ccc6oc7ccccc7c6c5c4c3)cc21]

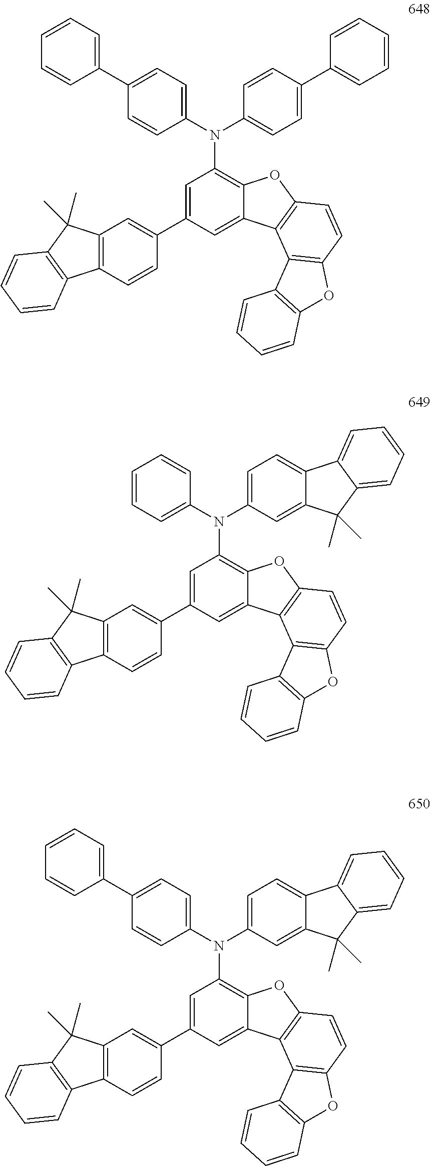 [SMILES: CC1(C)c2ccccc2-c2ccc(-c3cc(N(c4ccc(-c5ccccc5)cc4)c4ccc(-c5ccccc5)cc4)c4oc5ccc6oc7ccccc7c6c5c4c3)cc21 ; CC1(C)c2ccccc2-c2ccc(-c3cc(N(c4ccccc4)c4ccc5c(c4)C(C)(C)c4ccccc4-5)c4oc5ccc6oc7ccccc7c6c5c4c3)cc21 ; CC1(C)c2ccccc2-c2ccc(-c3cc(N(c4ccc(-c5ccccc5)cc4)c4ccc5c(c4)C(C)(C)c4ccccc4-5)c4oc5ccc6oc7ccccc7c6c5c4c3)cc21]

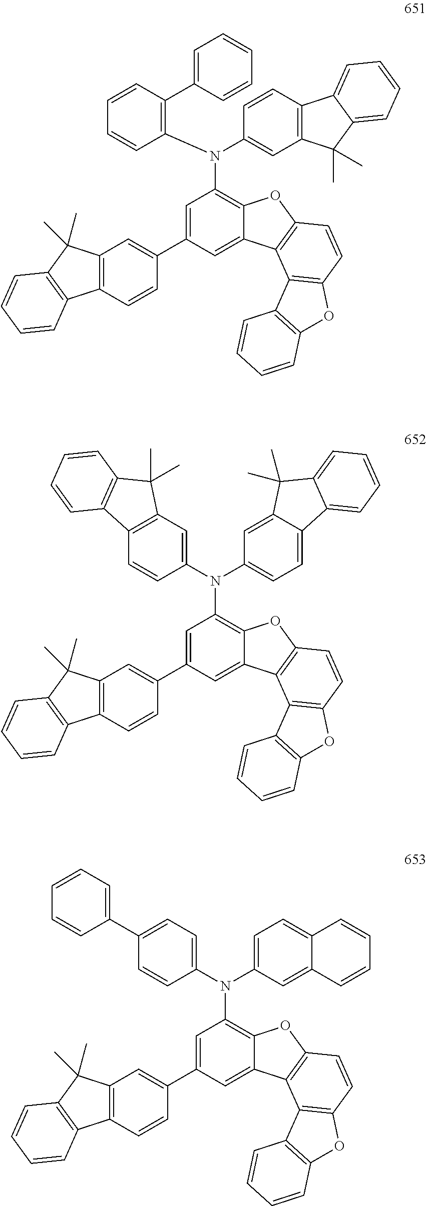 [SMILES: CC1(C)c2ccccc2-c2ccc(-c3cc(N(c4ccc5c(c4)C(C)(C)c4ccccc4-5)c4ccccc4-c4ccccc4)c4oc5ccc6oc7ccccc7c6c5c4c3)cc21 ; CC1(C)c2ccccc2-c2ccc(-c3cc(N(c4ccc5c(c4)C(C)(C)c4ccccc4-5)c4ccc5c(c4)C(C)(C)c4ccccc4-5)c4oc5ccc6oc7ccccc7c6c5c4c3)cc21 ; CC1(C)c2ccccc2-c2ccc(-c3cc(N(c4ccc(-c5ccccc5)cc4)c4ccc5ccccc5c4)c4oc5ccc6oc7ccccc7c6c5c4c3)cc21]

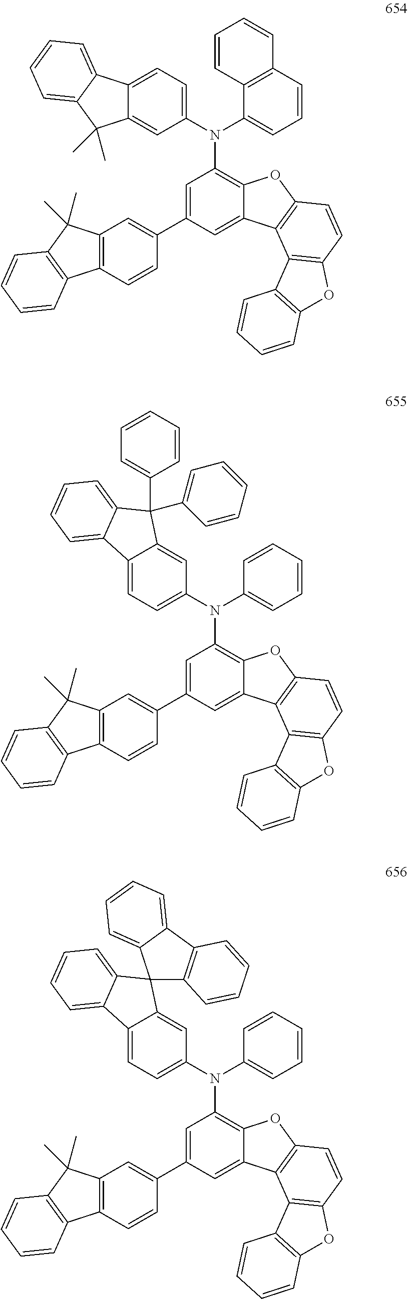 [SMILES: CC1(C)c2ccccc2-c2ccc(-c3cc(N(c4ccc5c(c4)C(C)(C)c4ccccc4-5)c4cccc5ccccc45)c4oc5ccc6oc7ccccc7c6c5c4c3)cc21 ; CC1(C)c2ccccc2-c2ccc(-c3cc(N(c4ccccc4)c4ccc5c(c4)C(c4ccccc4)(c4ccccc4)c4ccccc4-5)c4oc5ccc6oc7ccccc7c6c5c4c3)cc21 ; CC1(C)c2ccccc2-c2ccc(-c3cc(N(c4ccccc4)c4ccc5c(c4)C4(c6ccccc6-c6ccccc64)c4ccccc4-5)c4oc5ccc6oc7ccccc7c6c5c4c3)cc21]

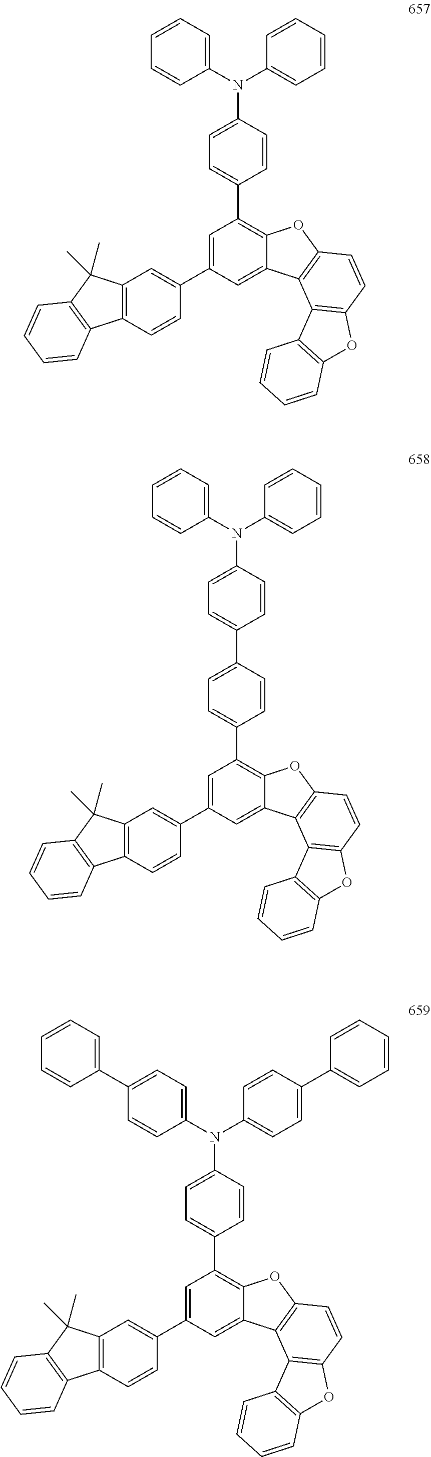 [SMILES: CC1(C)c2ccccc2-c2ccc(-c3cc(-c4ccc(N(c5ccccc5)c5ccccc5)cc4)c4oc5ccc6oc7ccccc7c6c5c4c3)cc21 ; CC1(C)c2ccccc2-c2ccc(-c3cc(-c4ccc(-c5ccc(N(c6ccccc6)c6ccccc6)cc5)cc4)c4oc5ccc6oc7ccccc7c6c5c4c3)cc21 ; CC1(C)c2ccccc2-c2ccc(-c3cc(-c4ccc(N(c5ccc(-c6ccccc6)cc5)c5ccc(-c6ccccc6)cc5)cc4)c4oc5ccc6oc7ccccc7c6c5c4c3)cc21]

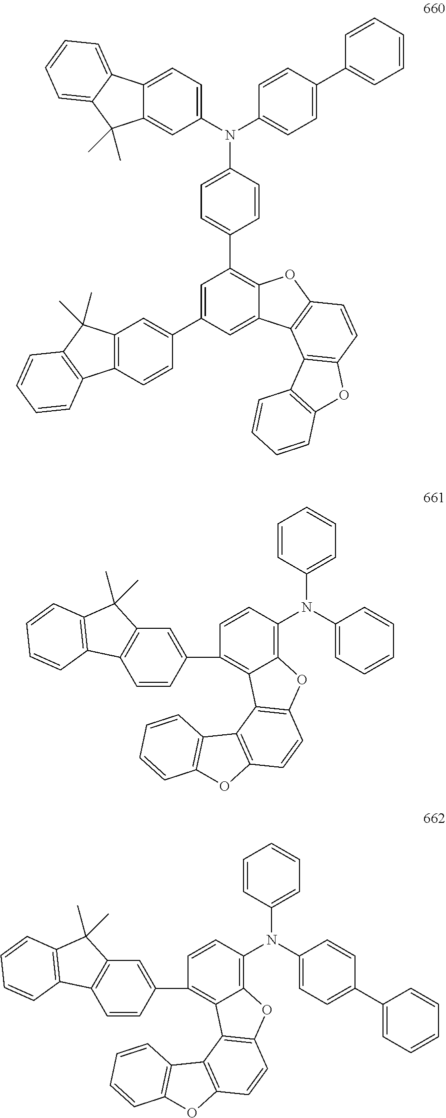 [SMILES: CC1(C)c2ccccc2-c2ccc(-c3cc(-c4ccc(N(c5ccc(-c6ccccc6)cc5)c5ccc6c(c5)C(C)(C)c5ccccc5-6)cc4)c4oc5ccc6oc7ccccc7c6c5c4c3)cc21 ; CC1(C)c2ccccc2-c2ccc(-c3ccc(N(c4ccccc4)c4ccccc4)c4oc5ccc6oc7ccccc7c6c5c34)cc21 ; CC1(C)c2ccccc2-c2ccc(-c3ccc(N(c4ccccc4)c4ccc(-c5ccccc5)cc4)c4oc5ccc6oc7ccccc7c6c5c34)cc21]

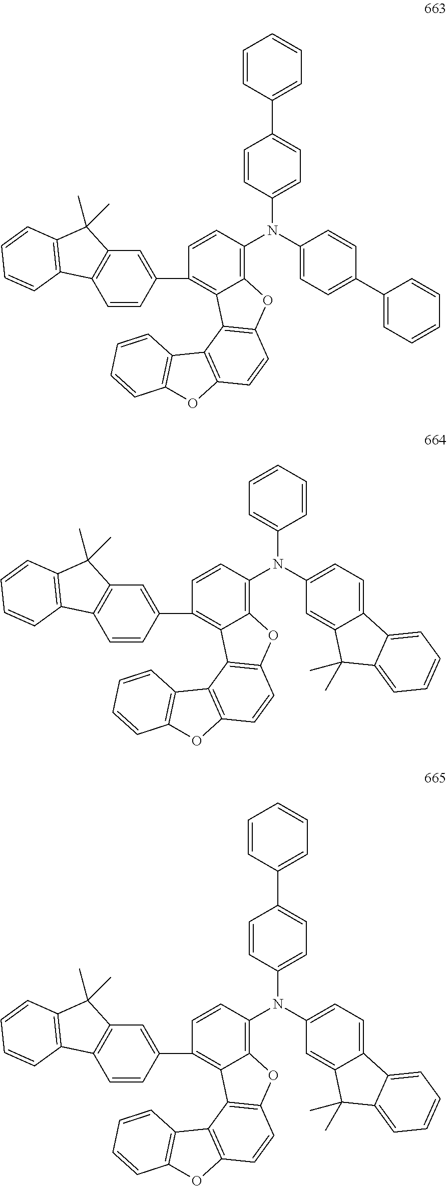 [SMILES: CC1(C)c2ccccc2-c2ccc(-c3ccc(N(c4ccc(-c5ccccc5)cc4)c4ccc(-c5ccccc5)cc4)c4oc5ccc6oc7ccccc7c6c5c34)cc21 ; CC1(C)c2ccccc2-c2ccc(-c3ccc(N(c4ccccc4)c4ccc5c(c4)C(C)(C)c4ccccc4-5)c4oc5ccc6oc7ccccc7c6c5c34)cc21 ; CC1(C)c2ccccc2-c2ccc(-c3ccc(N(c4ccc(-c5ccccc5)cc4)c4ccc5c(c4)C(C)(C)c4ccccc4-5)c4oc5ccc6oc7ccccc7c6c5c34)cc21]

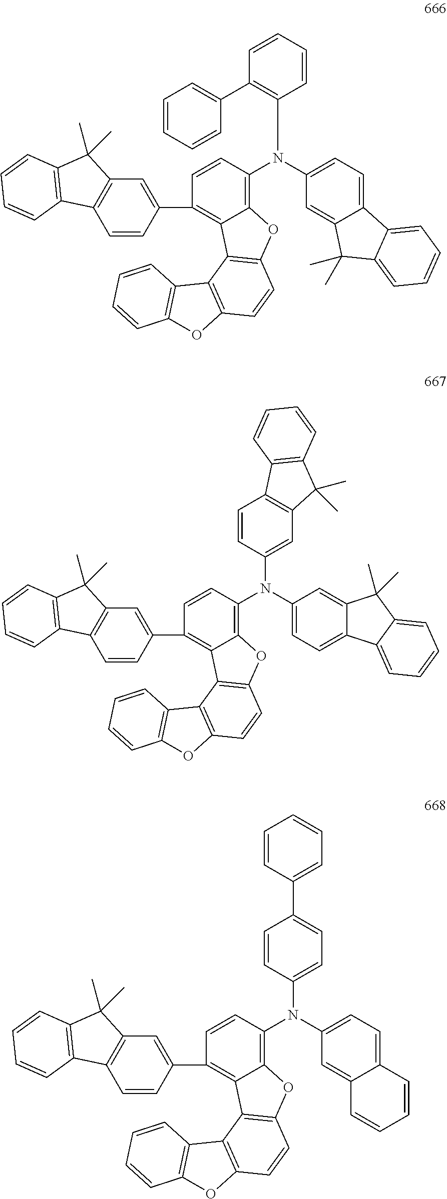 [SMILES: CC1(C)c2ccccc2-c2ccc(-c3ccc(N(c4ccc5c(c4)C(C)(C)c4ccccc4-5)c4ccccc4-c4ccccc4)c4oc5ccc6oc7ccccc7c6c5c34)cc21 ; CC1(C)c2ccccc2-c2ccc(-c3ccc(N(c4ccc5c(c4)C(C)(C)c4ccccc4-5)c4ccc5c(c4)C(C)(C)c4ccccc4-5)c4oc5ccc6oc7ccccc7c6c5c34)cc21 ; CC1(C)c2ccccc2-c2ccc(-c3ccc(N(c4ccc(-c5ccccc5)cc4)c4ccc5ccccc5c4)c4oc5ccc6oc7ccccc7c6c5c34)cc21]

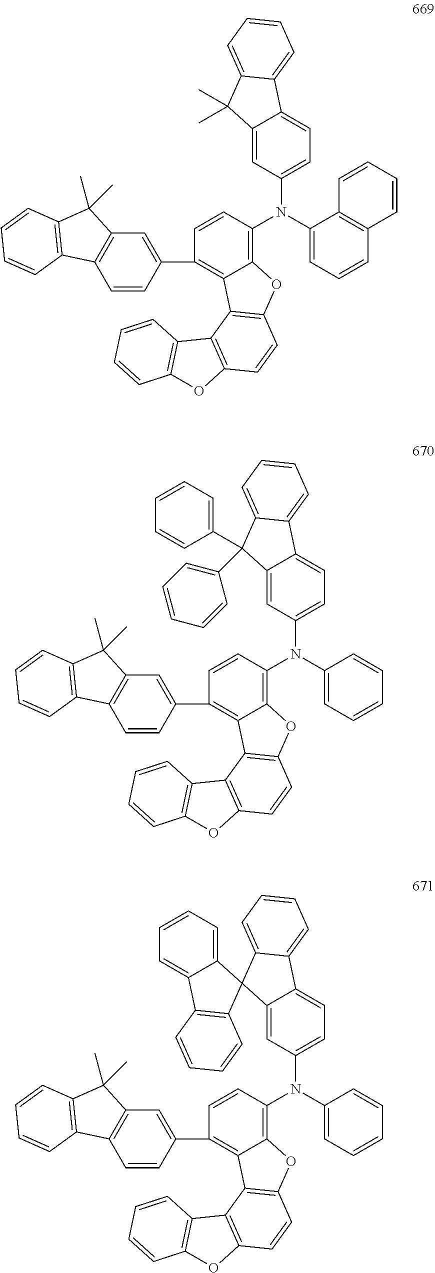 [SMILES: CC1(C)c2ccccc2-c2ccc(-c3ccc(N(c4ccc5c(c4)C(C)(C)c4ccccc4-5)c4cccc5ccccc45)c4oc5ccc6oc7ccccc7c6c5c34)cc21 ; CC1(C)c2ccccc2-c2ccc(-c3ccc(N(c4ccccc4)c4ccc5c(c4)C(c4ccccc4)(c4ccccc4)c4ccccc4-5)c4oc5ccc6oc7ccccc7c6c5c34)cc21 ; CC1(C)c2ccccc2-c2ccc(-c3ccc(N(c4ccccc4)c4ccc5c(c4)C4(c6ccccc6-c6ccccc64)c4ccccc4-5)c4oc5ccc6oc7ccccc7c6c5c34)cc21]

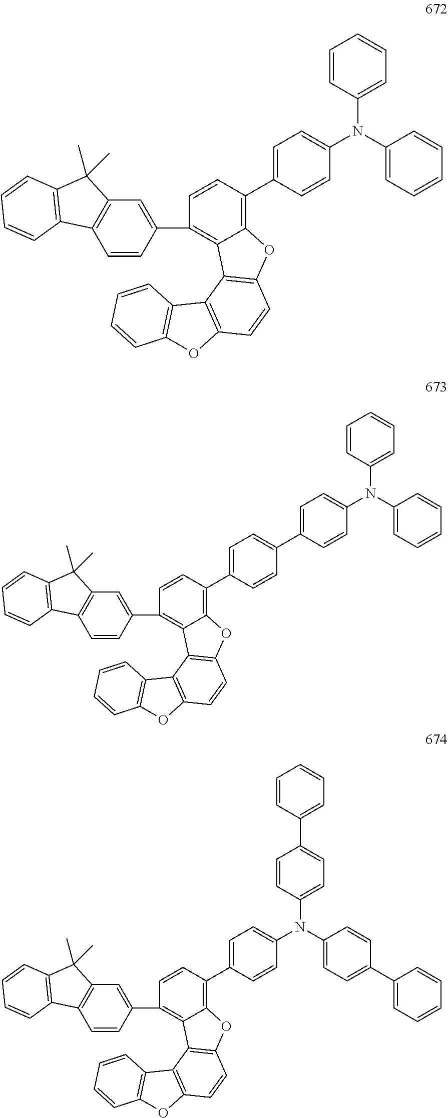 [SMILES: CC1(C)c2ccccc2-c2ccc(-c3ccc(-c4ccc(N(c5ccccc5)c5ccccc5)cc4)c4oc5ccc6oc7ccccc7c6c5c34)cc21 ; CC1(C)c2ccccc2-c2ccc(-c3ccc(-c4ccc(-c5ccc(N(c6ccccc6)c6ccccc6)cc5)cc4)c4oc5ccc6oc7ccccc7c6c5c34)cc21 ; CC1(C)c2ccccc2-c2ccc(-c3ccc(-c4ccc(N(c5ccc(-c6ccccc6)cc5)c5ccc(-c6ccccc6)cc5)cc4)c4oc5ccc6oc7ccccc7c6c5c34)cc21]

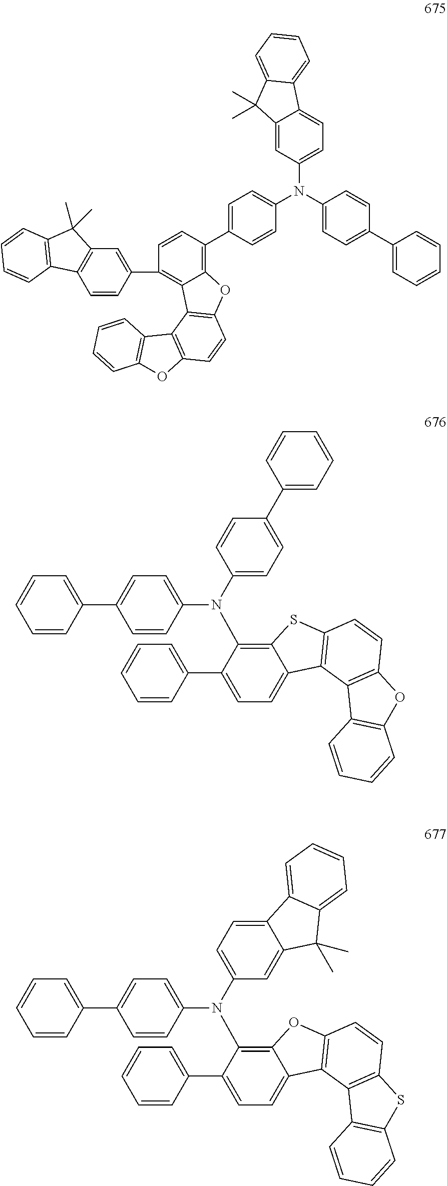 [SMILES: CC1(C)c2ccccc2-c2ccc(-c3ccc(-c4ccc(N(c5ccc(-c6ccccc6)cc5)c5ccc6c(c5)C(C)(C)c5ccccc5-6)cc4)c4oc5ccc6oc7ccccc7c6c5c34)cc21 ; c1ccc(-c2ccc(N(c3ccc(-c4ccccc4)cc3)c3c(-c4ccccc4)ccc4c3sc3ccc5oc6ccccc6c5c34)cc2)cc1 ; CC1(C)c2ccccc2-c2ccc(N(c3ccc(-c4ccccc4)cc3)c3c(-c4ccccc4)ccc4c3oc3ccc5sc6ccccc6c5c34)cc21]

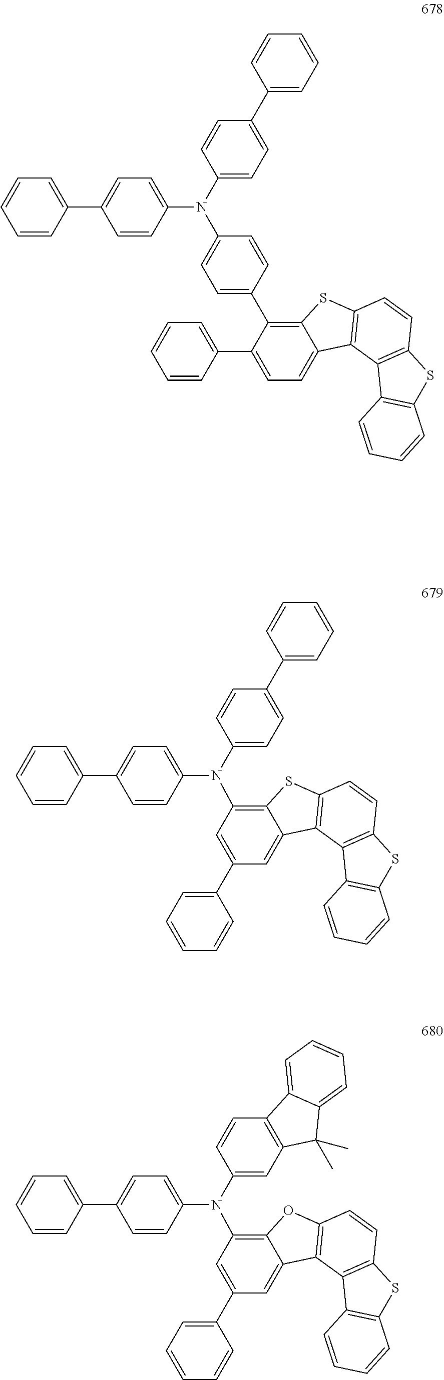 [SMILES: c1ccc(-c2ccc(N(c3ccc(-c4ccccc4)cc3)c3ccc(-c4c(-c5ccccc5)ccc5c4sc4ccc6sc7ccccc7c6c45)cc3)cc2)cc1 ; c1ccc(-c2ccc(N(c3ccc(-c4ccccc4)cc3)c3cc(-c4ccccc4)cc4c3sc3ccc5sc6ccccc6c5c34)cc2)cc1 ; CC1(C)c2ccccc2-c2ccc(N(c3ccc(-c4ccccc4)cc3)c3cc(-c4ccccc4)cc4c3oc3ccc5sc6ccccc6c5c34)cc21]

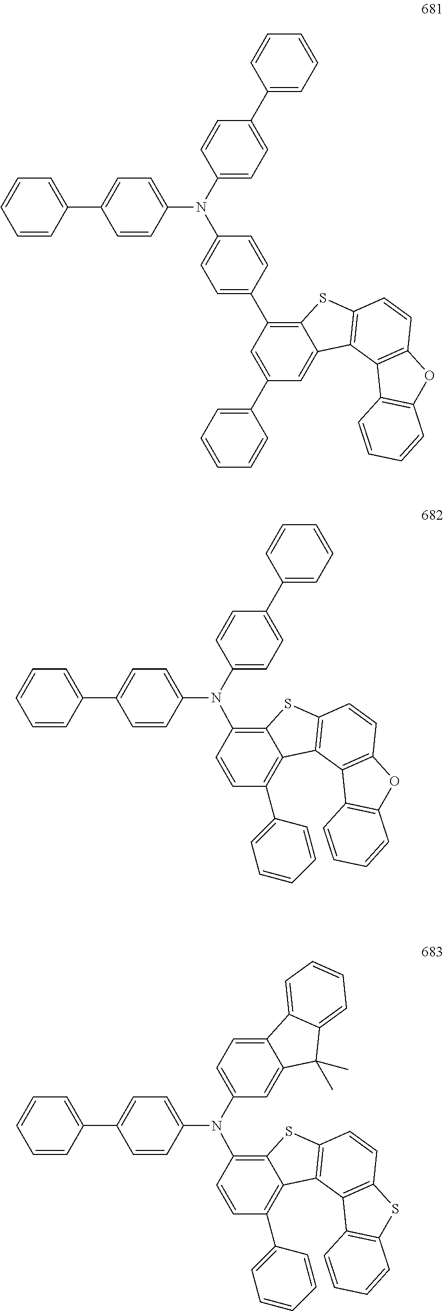 [SMILES: c1ccc(-c2ccc(N(c3ccc(-c4ccccc4)cc3)c3ccc(-c4cc(-c5ccccc5)cc5c4sc4ccc6oc7ccccc7c6c45)cc3)cc2)cc1 ; c1ccc(-c2ccc(N(c3ccc(-c4ccccc4)cc3)c3ccc(-c4ccccc4)c4c3sc3ccc5oc6ccccc6c5c34)cc2)cc1 ; CC1(C)c2ccccc2-c2ccc(N(c3ccc(-c4ccccc4)cc3)c3ccc(-c4ccccc4)c4c3sc3ccc5sc6ccccc6c5c34)cc21]

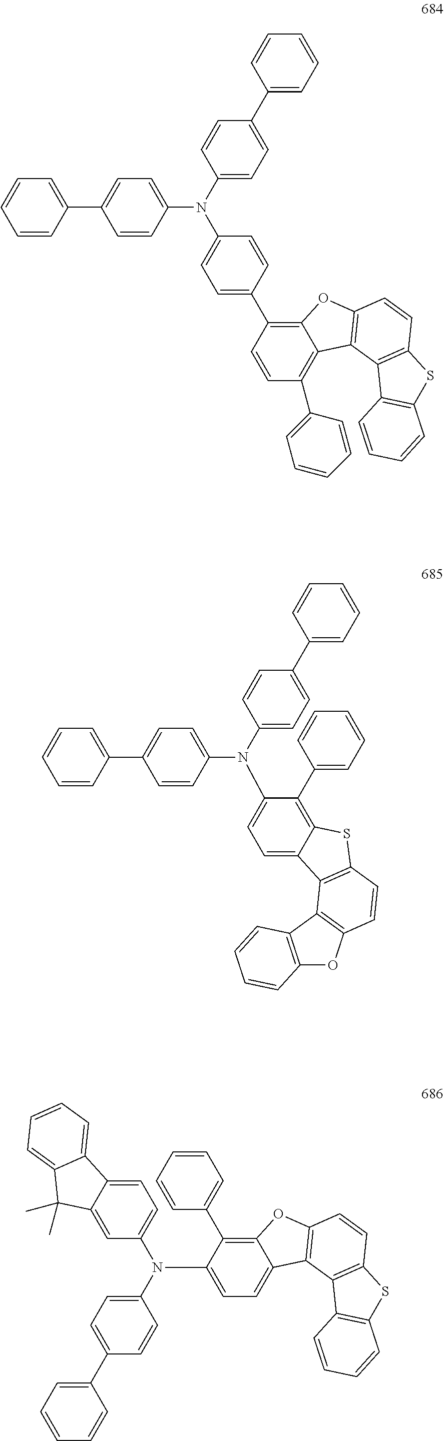 [SMILES: c1ccc(-c2ccc(N(c3ccc(-c4ccccc4)cc3)c3ccc(-c4ccc(-c5ccccc5)c5c4oc4ccc6sc7ccccc7c6c45)cc3)cc2)cc1 ; c1ccc(-c2ccc(N(c3ccc(-c4ccccc4)cc3)c3ccc4c(sc5ccc6oc7ccccc7c6c54)c3-c3ccccc3)cc2)cc1 ; CC1(C)c2ccccc2-c2ccc(N(c3ccc(-c4ccccc4)cc3)c3ccc4c(oc5ccc6sc7ccccc7c6c54)c3-c3ccccc3)cc21]

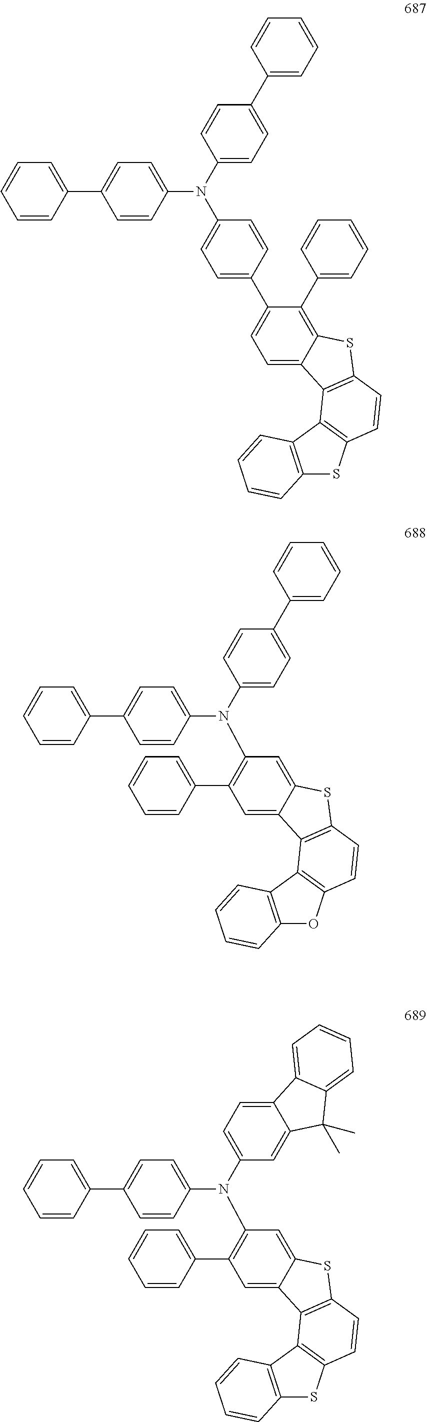

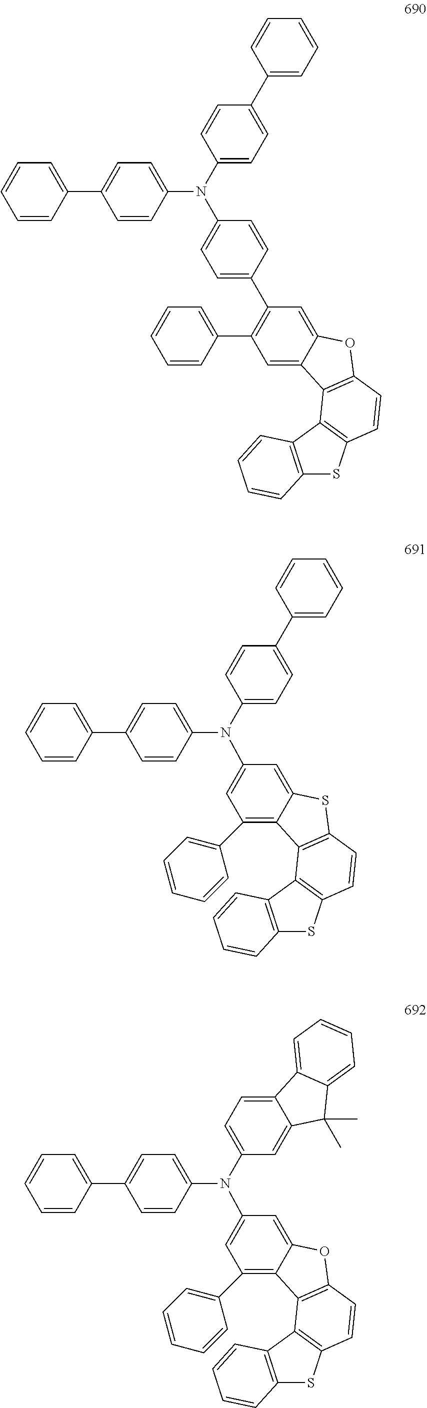 [SMILES: c1ccc(-c2ccc(N(c3ccc(-c4ccccc4)cc3)c3ccc(-c4cc5oc6ccc7sc8ccccc8c7c6c5cc4-c4ccccc4)cc3)cc2)cc1 ; c1ccc(-c2ccc(N(c3ccc(-c4ccccc4)cc3)c3cc(-c4ccccc4)c4c(c3)sc3ccc5sc6ccccc6c5c34)cc2)cc1 ; CC1(C)c2ccccc2-c2ccc(N(c3ccc(-c4ccccc4)cc3)c3cc(-c4ccccc4)c4c(c3)oc3ccc5sc6ccccc6c5c34)cc21]

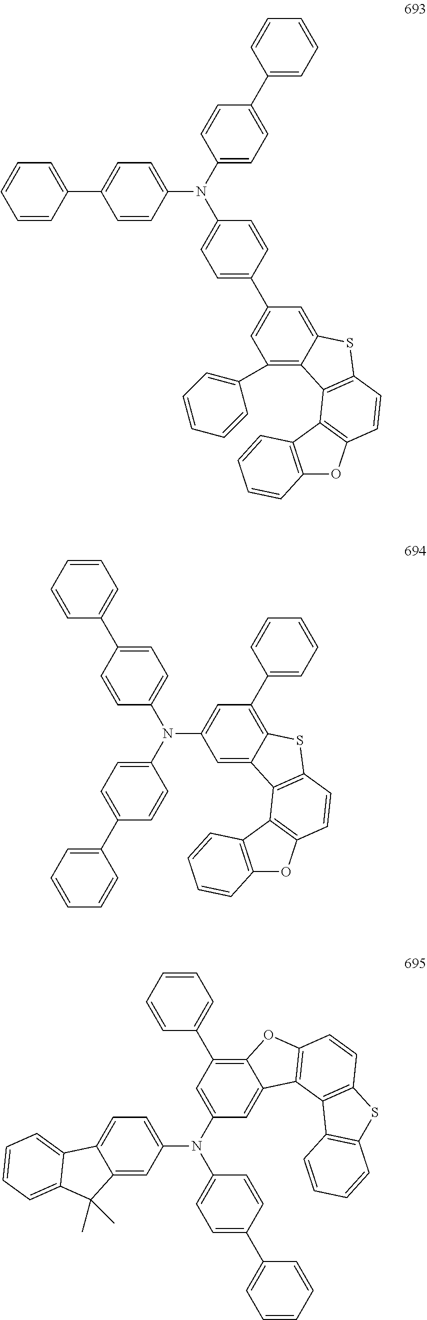 [SMILES: c1ccc(-c2ccc(N(c3ccc(-c4ccccc4)cc3)c3ccc(-c4cc(-c5ccccc5)c5c(c4)sc4ccc6oc7ccccc7c6c45)cc3)cc2)cc1 ; c1ccc(-c2ccc(N(c3ccc(-c4ccccc4)cc3)c3cc(-c4ccccc4)c4sc5ccc6oc7ccccc7c6c5c4c3)cc2)cc1 ; CC1(C)c2ccccc2-c2ccc(N(c3ccc(-c4ccccc4)cc3)c3cc(-c4ccccc4)c4oc5ccc6sc7ccccc7c6c5c4c3)cc21]

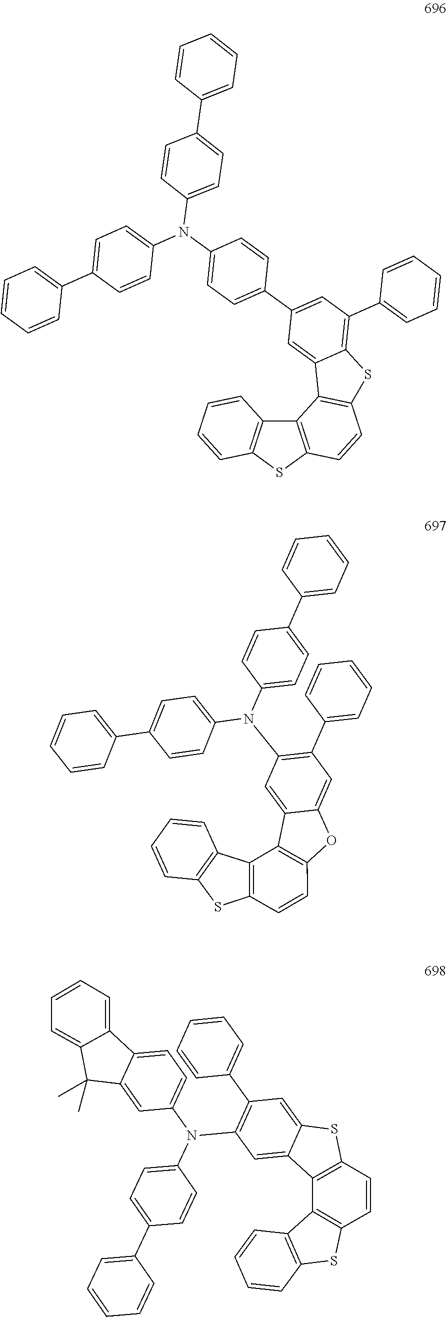 [SMILES: c1ccc(-c2ccc(N(c3ccc(-c4ccccc4)cc3)c3ccc(-c4cc(-c5ccccc5)c5sc6ccc7sc8ccccc8c7c6c5c4)cc3)cc2)cc1 ; c1ccc(-c2ccc(N(c3ccc(-c4ccccc4)cc3)c3cc4c(cc3-c3ccccc3)oc3ccc5sc6ccccc6c5c34)cc2)cc1 ; CC1(C)c2ccccc2-c2ccc(N(c3ccc(-c4ccccc4)cc3)c3cc4c(cc3-c3ccccc3)sc3ccc5sc6ccccc6c5c34)cc21]

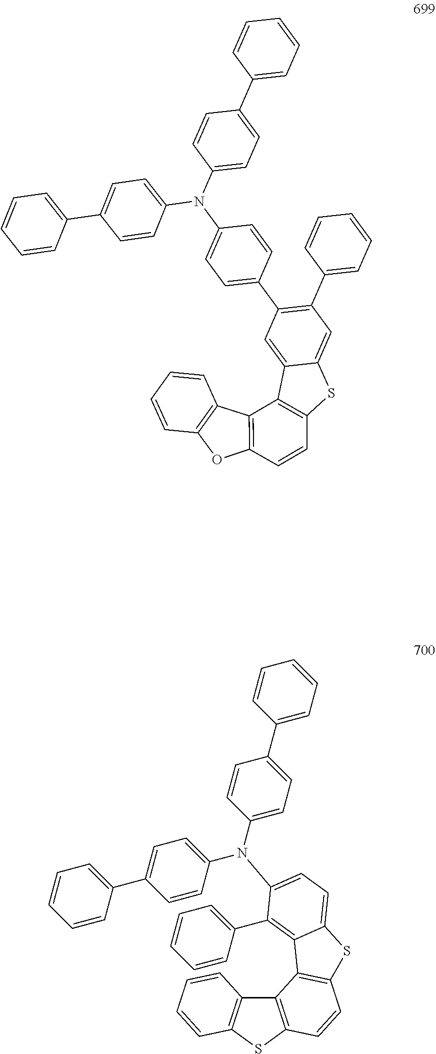 [SMILES: c1ccc(-c2ccc(N(c3ccc(-c4ccccc4)cc3)c3ccc(-c4cc5c(cc4-c4ccccc4)sc4ccc6oc7ccccc7c6c45)cc3)cc2)cc1 ; c1ccc(-c2ccc(N(c3ccc(-c4ccccc4)cc3)c3ccc4sc5ccc6sc7ccccc7c6c5c4c3-c3ccccc3)cc2)cc1]

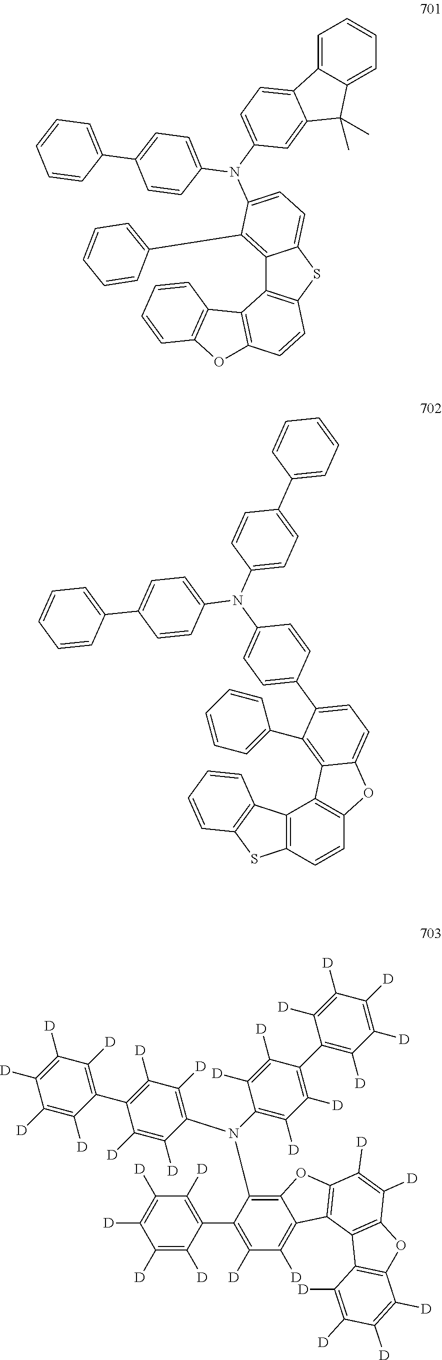 [SMILES: CC1(C)c2ccccc2-c2ccc(N(c3ccc(-c4ccccc4)cc3)c3ccc4sc5ccc6oc7ccccc7c6c5c4c3-c3ccccc3)cc21 ; c1ccc(-c2ccc(N(c3ccc(-c4ccccc4)cc3)c3ccc(-c4ccc5oc6ccc7sc8ccccc8c7c6c5c4-c4ccccc4)cc3)cc2)cc1 ; [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c(-c4c([2H])c([2H])c([2H])c([2H])c4[2H])c([2H])c([2H])c4c3oc3c([2H])c([2H])c5oc6c([2H])c([2H])c([2H])c([2H])c6c5c34)c([2H])c2[2H])c([2H])c1[2H]]

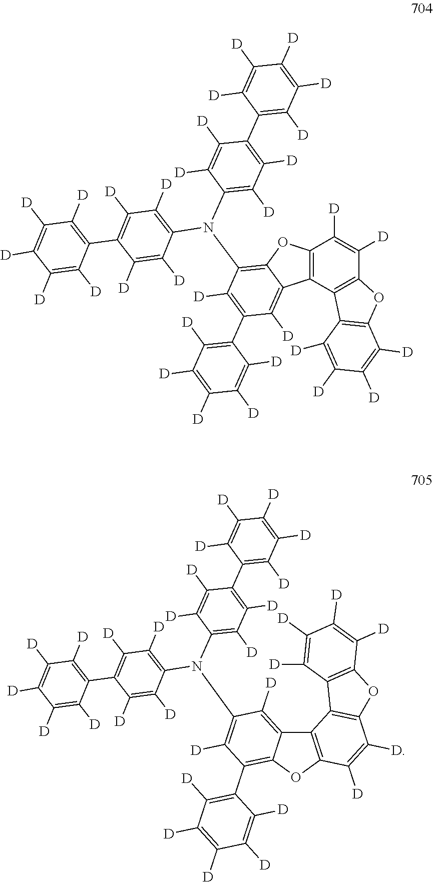

7. An organic light-emitting device comprising:
a first electrode;
a second electrode provided to face the first electrode; and
one or more organic layers provided between the first electrode and the second electrode, and
wherein one or more of the organic layers comprise the heterocyclic compound according to claim 1.

8. The organic light-emitting device according to claim 7, characterized in that the organic layer comprises one or more layers selected from the group consisting of a light-emitting layer, a hole injection layer, a hole transport layer, an electron injection layer, an electron transport layer, an electron-blocking layer, and a hole-blocking layer.

9. The organic light-emitting device according to claim 8, characterized in that the organic layer includes the hole transport layer, wherein the hole transport layer comprises the heterocyclic compound.

10. The organic light-emitting device according to claim 8, characterize in that the organic layer includes the electron-blocking layer, wherein the electron-blocking layer comprises the heterocyclic compound.

11. The organic light-emitting device according to claim 8, characterize in that the organic layer includes the light-emitting layer, wherein the light-emitting layer includes a host material, wherein the host material includes the heterocyclic alone, or includes the heterocyclic compound in combination with other host materials.

12. A composition for an organic layer of an organic light-emitting device, comprising the heterocyclic compound according to claim 1.

13. The composition for an organic layer according to claim 12, characterized in that the composition for an organic layer is use as one or more uses selected from a hole injection layer material, a hole transport layer material, an electron-blocking layer material, a light-emitting layer material, a hole-blocking layer material, an electron transport layer material, and an electron injection layer material of the organic light-emitting device.

14. The composition for an organic layer according to claim 13, characterized in that the composition for an organic layer is used as the hole transport layer material, the electron-blocking layer material, or the light-emitting layer material.

15. The composition for an organic layer according to claim 14, characterized in that the light-emitting layer material is a host material.
